SEQUENCE LISTING (269 sequences)

SEQ ID NO: 1 - protein, 6 aa, synthetic construct
NSMFDY

SEQ ID NO: 2 - protein, 7 aa, synthetic construct
ISSIKDK

SEQ ID NO: 3 - protein, 14 aa, synthetic construct
CAATRTSGTYKYIF

SEQ ID NO: 4 - protein, 5 aa, synthetic construct
MNHNY

SEQ ID NO: 5 - protein, 6 aa, synthetic construct
SVGAGI

SEQ ID NO: 6 - protein, 16 aa, synthetic construct
CASSPWGAGGTDTQYF

SEQ ID NO: 7 - protein, 139 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAATRTSGTYKYIFGTGTRLKVLA

SEQ ID NO: 8 - protein, 135 aa, synthetic construct
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSPWGAGGTDTQYFGPGTRLTVLE

SEQ ID NO: 9 - protein, 268 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAATRTSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPGKGSFGAFAGCFLASGMARFCPELWSMMSKTPLIGGLG

SEQ ID NO: 10 - protein, 313 aa, synthetic construct
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSPWGAGGTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 11 - protein, 6 aa, synthetic construct
VSNAYN

SEQ ID NO: 12 - protein, 4 aa, synthetic construct
GSKP

SEQ ID NO: 13 - protein, 15 aa, synthetic construct
CAVEDLNQAGTALIF

SEQ ID NO: 14 - protein, 5 aa, synthetic construct
SGHRS

SEQ ID NO: 15 - protein, 6 aa, synthetic construct
YFSETQ

SEQ ID NO: 16 - protein, 15 aa, synthetic construct
CASSLALGQGNQQFF

SEQ ID NO: 17 - protein, 129 aa, synthetic construct
MKSLRVLLVILWLQLSWVWSQKDQVFQPSTVASSEGAVVEIFCNHSVSNAYNFFWYLHFPGCAPRLLVKGSKPSQQGRYNMTYERFSSSLLILQVREADAAVYYCAVEDLNQAGTALIFGKGTTLSVSS

SEQ ID NO: 18 - protein, 134 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLALGQGNQQFFGPGTRLTVLE

SEQ ID NO: 19 - protein, 258 aa, synthetic construct
MKSLRVLLVILWLQLSWVWSQKDQVFQPSTVASSEGAVVEIFCNHSVSNAYNFFWYLHFPGCAPRLLVKGSKPSQQGRYNMTYERFSSSLLILQVREADAAVYYCAVEDLNQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPGKGSFGAFAGCFLASGMARFCPELWSMMSKTPLIGGLG

SEQ ID NO: 20 - protein, 312 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLALGQGNQQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 21 - protein, 5 aa, synthetic construct
TTLSN

SEQ ID NO: 22 - protein, 7 aa, synthetic construct
LVKSGEV

SEQ ID NO: 23 - protein, 12 aa, synthetic construct
CAGQTGANNLFF

SEQ ID NO: 24 - protein, 5 aa, synthetic construct
LGHNA

SEQ ID NO: 25 - protein, 6 aa, synthetic construct
YSLEER

SEQ ID NO: 26 - protein, 17 aa, synthetic construct
CASSQEVGTVPNQPQHF

SEQ ID NO: 27 - protein, 128 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGQTGANNLFFGTGTRLTVIP

SEQ ID NO: 28 - protein, 135 aa, synthetic construct
MGCRLLCCAVLCLLGAVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEVGTVPNQPQHFGDGTRLSIL

SEQ ID NO: 29 - protein, 257 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGQTGANNLFFGTGTRLTVIPYIQNPDPAVYQLRDSKSSDKSVCLFTDFDSQTNVSQSKDSDVYITDKTVLDMRSMDFKSNSAVAWSNKSDFACANAFNNSIIPEDTFFPSPGKGSFGAFAGCFLASGMARFCPELWSMMSKTPLIGGLG

SEQ ID NO: 30 - protein, 312 aa, synthetic construct
MGCRLLCCAVLCLLGAVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEVGTVPNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 31 - DNA, 18 bp, synthetic construct
  source 1..18: mol_type = other DNA
aacagcatgtttgattat

SEQ ID NO: 32 - DNA, 21 bp, synthetic construct
  source 1..21: mol_type = other DNA
ataagttccattaaggataaa

SEQ ID NO: 33 - DNA, 42 bp, synthetic construct
  source 1..42: mol_type = other DNA
tgtgcagcaacccgtacctcaggaacctacaaatacatcttt

SEQ ID NO: 34 - DNA, 15 bp, synthetic construct
  source 1..15: mol_type = other DNA
atgaaccataactac

SEQ ID NO: 35 - DNA, 18 bp, synthetic construct
  source 1..18: mol_type = other DNA
tcagttggtgctggtatc

SEQ ID NO: 36 - DNA, 48 bp, synthetic construct
  source 1..48: mol_type = other DNA
tgtgccagcagtccgtggggggcaggcggcacagatacgcagtatttt

SEQ ID NO: 37 - DNA, 418 bp, synthetic construct
  source 1..418: mol_type = other DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaacccgtacctcaggaacctacaaatacatctttggaacaggcaccaggctgaaggttttagcaa

SEQ ID NO: 38 - DNA, 402 bp, synthetic construct
  source 1..402: mol_type = other DNA
atgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagtccgtggggggcaggcggcacagatacgcagtattttggcccaggcacccggctgacagtgctc

SEQ ID NO: 39 - DNA, 804 bp, synthetic construct
  source 1..804: mol_type = other DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaacccgtacctcaggaacctacaaatacatctttggaacaggcaccaggctgaaggttttagcaaatatccagaaccctgaccctgcagtatatcagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggc

SEQ ID NO: 40 - DNA, 939 bp, synthetic construct
  source 1..939: mol_type = other DNA
atgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagtccgtggggggcaggcggcacagatacgcagtattttggcccaggcacccggctgacagtgctcgaggaccttaagaatgtgttccctcccgaggtggctgtcttcgaaccaagcgaagccgagatctctcacacacaaaaggctactctcgtgtgtctggcaacgggtttttaccctgatcatgtagagttgtcttggtgggttaacggcaaagaagtacactccggtgtaagcaccgatcctcagccacttaaggaacaacccgcactcaacgactccagatactgtttgagttctaggctgagagtctcagcgacgttttggcagaacccacggaatcatttcagatgtcaggtccagttttacgggttgagcgagaacgacgagtggacacaggatcgggctaaaccagtgacccagattgtatcagccgaagcatggggaagggcagattgcggctttacctcagagtcatatcaacagggcgttttgtctgcaacaatcctttatgagatcctgcttggtaaggcgactctgtatgccgtgcttgttagtgccctggttctgatggcaatggtaaaaagaaaggactctagggga

SEQ ID NO: 41 - DNA, 18 bp, synthetic construct
  source 1..18: mol_type = other DNA
gtgtccaacgcctacaat

SEQ ID NO: 42 - DNA, 12 bp, synthetic construct
  source 1..12: mol_type = other DNA
ggctctaagccc

SEQ ID NO: 43 - DNA, 45 bp, synthetic construct
  source 1..45: mol_type = other DNA
tgtgccgtggaggatctgaatcaggccggcacagccctgattttc

SEQ ID NO: 44 - DNA, 15 bp, synthetic construct
  source 1..15: mol_type = other DNA
tccggacaccgctcc

SEQ ID NO: 45 - DNA, 18 bp, synthetic construct
  source 1..18: mol_type = other DNA
tatttctctgagacacag

SEQ ID NO: 46 - DNA, 45 bp, synthetic construct
  source 1..45: mol_type = other DNA
tgcgcaagctccctggccctgggacaggggaatcagcagtttttc

SEQ ID NO: 47 - DNA, 387 bp, synthetic construct
  source 1..387: mol_type = other DNA
atgaagagcctgcgcgtgctgctggtcatcctgtggctgcaattgagttgggtgtggagccagaaggaccaggtgtttcagcctagcactgtcgcatcatcagagggggccgtcgtggaaatcttttgtaatcacagcgtgtccaacgcctacaatttcttttggtatctgcacttcccaggatgcgcacctaggctgctggtgaagggctctaagcccagccagcagggccggtacaacatgacctatgagcggttcagctcctctctgctgatcctgcaggtgcgggaggcagacgcagccgtgtactattgtgccgtggaggatctgaatcaggccggcacagccctgattttcggcaaaggaacaaccctgagcgtgagcagc

SEQ ID NO: 48 - DNA, 400 bp, synthetic construct
  source 1..400: mol_type = other DNA
atgggatcaagactgctgtgctgggtgctgctgtgcctgctgggagccggacctgtgaaagccggggtgactcagactccacgatacctgatcaagaccaggggccagcaggtgacactgtcttgcagcccaatctccggacaccgctccgtgtcttggtaccagcagacccctggacagggactgcagttcctgtttgagtatttctctgagacacagcggaacaagggcaatttccccggccggtttagcggcagacagtttagcaactccaggtctgagatgaatgtgagcaccctggagctgggcgactccgccctgtacctgtgcgcaagctccctggccctgggacaggggaatcagcagtttttcggacccggaacaaggctgaccgtgctgg

SEQ ID NO: 49 - DNA, 774 bp, synthetic construct
  source 1..774: mol_type = other DNA
atgaagagcctgcgcgtgctgctggtcatcctgtggctgcaattgagttgggtgtggagccagaaggaccaggtgtttcagcctagcactgtcgcatcatcagagggggccgtcgtggaaatcttttgtaatcacagcgtgtccaacgcctacaatttcttttggtatctgcacttcccaggatgcgcacctaggctgctggtgaagggctctaagcccagccagcagggccggtacaacatgacctatgagcggttcagctcctctctgctgatcctgcaggtgcgggaggcagacgcagccgtgtactattgtgccgtggaggatctgaatcaggccggcacagccctgattttcggcaaaggaacaaccctgagcgtgagcagcaatatccagaaccctgaccctgcagtatatcagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggc

SEQ ID NO: 50 - DNA, 936 bp, synthetic construct
  source 1..936: mol_type = other DNA
atgggatcaagactgctgtgctgggtgctgctgtgcctgctgggagccggacctgtgaaagccggggtgactcagactccacgatacctgatcaagaccaggggccagcaggtgacactgtcttgcagcccaatctccggacaccgctccgtgtcttggtaccagcagacccctggacagggactgcagttcctgtttgagtatttctctgagacacagcggaacaagggcaatttccccggccggtttagcggcagacagtttagcaactccaggtctgagatgaatgtgagcaccctggagctgggcgactccgccctgtacctgtgcgcaagctccctggccctgggacaggggaatcagcagtttttcggacccggaacaaggctgaccgtgctggaggaccttaagaatgtgttccctcccgaggtggctgtcttcgaaccaagcgaagccgagatctctcacacacaaaaggctactctcgtgtgtctggcaacgggtttttaccctgatcatgtagagttgtcttggtgggttaacggcaaagaagtacactccggtgtatctaccgatcctcagccacttaaggaacaacccgcactcaacgactccagatactgtttgagttctaggctgagagtctcagcgacgttttggcagaacccacggaatcatttcagatgtcaggtccagttttacgggttgagcgagaacgacgagtggacacaggatcgggctaaaccagtgacccagattgtatcagccgaagcatggggaagggcagattgcggctttacctcagagtcatatcaacagggcgttttgtctgcaacaatcctttatgagatcctgcttggtaaggcgactctgtatgccgtgcttgttagtgccctggttctgatggcaatggtaaaaagaaaggactctagggga

SEQ ID NO: 51 - DNA, 15 bp, synthetic construct
  source 1..15: mol_type = other DNA
actactttaagcaat

SEQ ID NO: 52 - DNA, 21 bp, synthetic construct
  source 1..21: mol_type = other DNA
ttagtgaagagtggagaagtg

SEQ ID NO: 53 - DNA, 36 bp, synthetic construct
  source 1..36: mol_type = other DNA
tgtgcaggtcaaactggggcaaacaacctcttcttt

SEQ ID NO: 54 - DNA, 15 bp, synthetic construct
  source 1..15: mol_type = other DNA
ctgggtcataacgct

SEQ ID NO: 55 - DNA, 18 bp, synthetic construct
  source 1..18: mol_type = other DNA
tacagtcttgaagaacgg

SEQ ID NO: 56 - DNA, 51 bp, synthetic construct
  source 1..51: mol_type = other DNA
tgcgccagcagccaagaagtaggtacagtccccaatcagccccagcatttt

SEQ ID NO: 57 - DNA, 385 bp, synthetic construct
  source 1..385: mol_type = other DNA
atgctactcatcacatcaatgttggtcttatggatgcaattgtcacaggtgaatggacaacaggtaatgcaaattcctcagtaccagcatgtacaagaaggagaggacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcaggtcaaactggggcaaacaacctcttctttgggactggaacgagactcaccgttattccct

SEQ ID NO: 58 - DNA, 406 bp, synthetic construct
  source 1..406: mol_type = other DNA
atgggctgcaggctgctctgctgtgcggttctctgtctcctgggagcggtccccatggaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaagaagtaggtacagtccccaatcagccccagcattttggtgatgggactcgactctccatcctag

SEQ ID NO: 59 - DNA, 771 bp, synthetic construct
  source 1..771: mol_type = other DNA
atgctactcatcacatcaatgttggtcttatggatgcaattgtcacaggtgaatggacaacaggtaatgcaaattcctcagtaccagcatgtacaagaaggagaggacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcaggtcaaactggggcaaacaacctcttctttgggactggaacgagactcaccgttattccctatatccagaaccctgaccctgcagtatatcagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggc

SEQ ID NO: 60 - DNA, 936 bp, synthetic construct
  source 1..936: mol_type = other DNA
atgggctgcaggctgctctgctgtgcggttctctgtctcctgggagcggtccccatggaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaagaagtaggtacagtccccaatcagccccagcattttggtgatgggactcgactctccatcctagaggaccttaataaggtgttccctcccgaggtggctgtcttcgaaccaagcgaagccgagatctctcacacacaaaaggctactctcgtgtgtctggcaacgggttttttccctgatcatgtagagttgtcttggtgggttaacggcaaagaagtacactccggtgtaagcaccgatcctcagccacttaaggaacaacccgcactcaacgactccagatactgtttgagttctaggctgagagtctcagcgacgttttggcagaacccacggaatcatttcagatgtcaggtccagttttacgggttgagcgagaacgacgagtggacacaggatcgggctaaaccagtgacccagattgtatcagccgaagcatggggaagggcagattgcggctttacctcagtttcatatcaacagggcgttttgtctgcaacaatcctttatgagatcctgcttggtaaggcgactctgtatgccgtgcttgttagtgccctggttctgatggcaatggtaaaaagaaaggacttt

SEQ ID NO: 61 - protein, 20 aa, synthetic construct
MPLGLLWLGLALLGALHAQA

SEQ ID NO: 62 - protein, 108 aa, synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGE

SEQ ID NO: 63 - protein, 16 aa, synthetic construct
GSNTSKENPFLFALEA

SEQ ID NO: 64 - protein, 21 aa, synthetic construct
VVISVGSMGLIISLLCVYFWL

SEQ ID NO: 65 - protein, 86 aa, synthetic construct
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 66 - protein, 231 aa, synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 67 - protein, 89 aa, synthetic construct
EMWHEGLEEASRLYFGERNVKGMFEVLEPLHAMMERGPQTLKETSFNQAYGRDLMEAQEWCRKYMKSGNVKDLTQAWDLYYHVFRRISK

SEQ ID NO: 68 - protein, 4 aa, synthetic construct
GKDT

SEQ ID NO: 69 - protein, 25 aa, synthetic construct
IPWLGHLLVGLSGAFGFIILVYLLI

SEQ ID NO: 70 - protein, 286 aa, synthetic construct
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 71 - protein, 404 aa, synthetic construct
EMWHEGLEEASRLYFGERNVKGMFEVLEPLHAMMERGPQTLKETSFNQAYGRDLMEAQEWCRKYMKSGNVKDLTQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 72 - protein, 89 aa, synthetic construct; identical to SEQ ID NO: 67

SEQ ID NO: 73 - DNA, 60 bp, synthetic construct
  source 1..60: mol_type = other DNA
atgcctctgggcctgctgtggctgggcctggccctgctgggcgccctgcacgcccaggcc

SEQ ID NO: 74 - DNA, 324 bp, synthetic construct
  source 1..324: mol_type = other DNA
ggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgag

SEQ ID NO: 75 - DNA, 48 bp, synthetic construct
  source 1..48: mol_type = other DNA
ggatccaacacatcaaaagagaacccctttctgttcgcattggaggcc

SEQ ID NO: 76 - DNA, 63 bp, synthetic construct
  source 1..63: mol_type = other DNA
gtagtcatatctgttggatccatgggacttattatctccctgttgtgtgtgtacttctggctg

SEQ ID NO: 77 - DNA, 258 bp, synthetic construct
  source 1..258: mol_type = other DNA
gaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaact

SEQ ID NO: 78 - DNA, 693 bp, synthetic construct
  source 1..693: mol_type = other DNA
ggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaact

SEQ ID NO: 79 - DNA, 267 bp, synthetic construct
  source 1..267: mol_type = other DNA
gagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaag

SEQ ID NO: 80 - DNA, 12 bp, synthetic construct
  source 1..12: mol_type = other DNA
ggcaaagacacg

SEQ ID NO: 81 - DNA, 75 bp, synthetic construct
  source 1..75: mol_type = other DNA
attccgtggcttgggcatctgctcgttgggctgagtggtgcgtttggtttcatcatcttggtctatctcttgatc

SEQ ID NO: 82 - DNA, 858 bp, synthetic construct
  source 1..858: mol_type = other DNA
aattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgta

SEQ ID NO: 83 - DNA, 1212 bp, synthetic construct
  source 1..1212: mol_type = other DNA
gagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgta

SEQ ID NO: 84 - DNA, 267 bp, synthetic construct
  source 1..267: mol_type = other DNA
gaaatgtggcacgaaggactcgaggaagccagtcggctgtattttggcgagcggaatgtgaaagggatgtttgaagtgctcgagcctctccacgctatgatggaacggggaccccagactctcaaagaaaccagctttaatcaggcttacggacgcgacctcatggaagctcaagaatggtgtagaaagtatatgaagagtggcaacgtgaaagatctgacacaagcctgggatctctattatcacgtgttcagacgcatcagcaaa

SEQ ID NO: 85 - DNA, 346 bp, synthetic construct
  source 1..346: mol_type = other DNA
cctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcc

SEQ ID NO: 86 - DNA, 347 bp, synthetic construct
  source 1..347: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 87 - DNA, 116 bp, synthetic construct
  source 1..116: mol_type = other DNA
gcctggagacgccatccacgctgttttgacttccatagaaggatctcgccgccaccatgcctctgggcctgctgtggctgggcctggccctgctgggcgccctgcacgcccaggcc

SEQ ID NO: 88 - DNA, 693 bp, synthetic construct; identical to SEQ ID NO: 78
  source 1..693: mol_type = other DNA

SEQ ID NO: 89 - DNA, 54 bp, synthetic construct
  source 1..54: mol_type = other DNA
gagggcagaggaagtctgctaacatgcggtgacgtcgaggagaatcctggacct

SEQ ID NO: 90 - DNA, 939 bp, synthetic construct; identical to SEQ ID NO: 40
  source 1..939: mol_type = other DNA

SEQ ID NO: 91 - DNA, 57 bp, synthetic construct
  source 1..57: mol_type = other DNA
gctactaacttcagcctgctgaagcaggctggagacgtggaggagaaccctggacct

SEQ ID NO: 92 - DNA, 447 bp, synthetic construct
  source 1..447: mol_type = other DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaacccgtacctcaggaacctacaaatacatctttggaacaggcaccaggctgaaggttttagcaaatatccagaaccctgaccctgcagtatat

SEQ ID NO: 93 - DNA, 357 bp, synthetic construct
  source 1..357: mol_type = other DNA
cagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggc

SEQ ID NO: 94 - DNA, 2673 bp, synthetic construct
  source 1..2673: mol_type = other DNA
tagaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggatccggcgagggcagaggaagtctgctaacatgcggtgacgtcgaggagaatcctggacctatgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagtccgtggggggcaggcggcacagatacgcagtattttggcccaggcacccggctgacagtgctcgaggaccttaagaatgtgttccctcccgaggtggctgtcttcgaaccaagcgaagccgagatctctcacacacaaaaggctactctcgtgtgtctggcaacgggtttttaccctgatcatgtagagttgtcttggtgggttaacggcaaagaagtacactccggtgtaagcaccgatcctcagccacttaaggaacaacccgcactcaacgactccagatactgtttgagttctaggctgagagtctcagcgacgttttggcagaacccacggaatcatttcagatgtcaggtccagttttacgggttgagcgagaacgacgagtggacacaggatcgggctaaaccagtgacccagattgtatcagccgaagcatggggaagggcagattgcggctttacctcagagtcatatcaacagggcgttttgtctgcaacaatcctttatgagatcctgcttggtaaggcgactctgtatgccgtgcttgttagtgccctggttctgatggcaatggtaaaaagaaaggactctaggggaggttctggcgctactaacttcagcctgctgaagcaggctggagacgtggaggagaaccctggacctatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaacccgtacctcaggaacctacaaatacatctttggaacaggcaccaggctgaaggttttagcaaatatccagaaccctgaccctgcagtatat

SEQ ID NO: 95 - DNA, 3376 bp, synthetic construct
  source 1..3376: mol_type = other DNA
cctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcctagaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggatccggcgagggcagaggaagtctgctaacatgcggtgacgtcgaggagaatcctggacctatgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagtccgtggggggcaggcggcacagatacgcagtattttggcccaggcacccggctgacagtgctcgaggaccttaagaatgtgttccctcccgaggtggctgtcttcgaaccaagcgaagccgagatctctcacacacaaaaggctactctcgtgtgtctggcaacgggtttttaccctgatcatgtagagttgtcttggtgggttaacggcaaagaagtacactccggtgtaagcaccgatcctcagccacttaaggaacaacccgcactcaacgactccagatactgtttgagttctaggctgagagtctcagcgacgttttggcagaacccacggaatcatttcagatgtcaggtccagttttacgggttgagcgagaacgacgagtggacacaggatcgggctaaaccagtgacccagattgtatcagccgaagcatggggaagggcagattgcggctttacctcagagtcatatcaacagggcgttttgtctgcaacaatcctttatgagatcctgcttggtaaggcgactctgtatgccgtgcttgttagtgccctggttctgatggcaatggtaaaaagaaaggactctaggggaggttctggcgctactaacttcagcctgctgaagcaggctggagacgtggaggagaaccctggacctatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaacccgtacctcaggaacctacaaatacatctttggaacaggcaccaggctgaaggttttagcaaatatccagaaccctgaccctgcagtatatcagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggc

SEQ ID NO: 96 - DNA, 500 bp, synthetic construct
  source 1..500: mol_type = other DNA
aacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtg

SEQ ID NO: 97 - DNA, 15 bp, synthetic construct
  source 1..15: mol_type = other DNA
tagcagcttagatac

SEQ ID NO: 98 - DNA, 347 bp, synthetic construct; identical to SEQ ID NO: 86
  source 1..347: mol_type = other DNA

SEQ ID NO: 99 - DNA, 116 bp, synthetic construct; identical to SEQ ID NO: 87
  source 1..116: mol_type = other DNA

SEQ ID NO: 100 - DNA, 693 bp, synthetic construct; identical to SEQ ID NO: 78
  source 1..693: mol_type = other DNA

SEQ ID NO: 101 - DNA, 54 bp, synthetic construct; identical to SEQ ID NO: 89
  source 1..54: mol_type = other DNA

SEQ ID NO: 102 - DNA, 939 bp, synthetic construct; identical to SEQ ID NO: 40
  source 1..939: mol_type = other DNA

SEQ ID NO: 103 - DNA, 57 bp, synthetic construct; identical to SEQ ID NO: 91
  source 1..57: mol_type = other DNA

SEQ ID NO: 104 - DNA, 459 bp, synthetic construct
  source 1..459: mol_type = other DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaacccgtacctcaggaacctacaaatacatctttggaacaggcaccaggctgaaggttttagcaaatatccagaaccctgaccctgcagtatatcagctgagagac

SEQ ID NO: 105 - DNA, 500 bp, synthetic construct
  source 1..500: mol_type = other DNA
tctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctg

SEQ ID NO: 106 - DNA, 2698 bp, synthetic construct
  source 1..2698: mol_type = other DNA
tagcagcttagatacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggatccggcgagggcagaggaagtctgctaacatgcggtgacgtcgaggagaatcctggacctatgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagtccgtggggggcaggcggcacagatacgcagtattttggcccaggcacccggctgacagtgctcgaggaccttaagaatgtgttccctcccgaggtggctgtcttcgaaccaagcgaagccgagatctctcacacacaaaaggctactctcgtgtgtctggcaacgggtttttaccctgatcatgtagagttgtcttggtgggttaacggcaaagaagtacactccggtgtaagcaccgatcctcagccacttaaggaacaacccgcactcaacgactccagatactgtttgagttctaggctgagagtctcagcgacgttttggcagaacccacggaatcatttcagatgtcaggtccagttttacgggttgagcgagaacgacgagtggacacaggatcgggctaaaccagtgacccagattgtatcagccgaagcatggggaagggcagattgcggctttacctcagagtcatatcaacagggcgttttgtctgcaacaatcctttatgagatcctgcttggtaaggcgactctgtatgccgtgcttgttagtgccctggttctgatggcaatggtaaaaagaaaggactctaggggaggttctggcgctactaacttcagcctgctgaagcaggctggagacgtggaggagaaccctggacctatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaacccgtacctcaggaacctacaaatacatctttggaacaggcaccaggctgaaggttttagcaaatatccagaaccctgaccctgcagtatatcagctgagagac

SEQ ID NO: 107 - DNA, 3698 bp, synthetic construct
  source 1..3698: mol_type = other DNA
aacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtagcagcttagatacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggatccggcgagggcagaggaagtctgctaacatgcggtgacgtcgaggagaatcctggacctatgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagtccgtggggggcaggcggcacagatacgcagtattttggcccaggcacccggctgacagtgctcgaggaccttaagaatgtgttccctcccgaggtggctgtcttcgaaccaagcgaagccgagatctctcacacacaaaaggctactctcgtgtgtctggcaacgggtttttaccctgatcatgtagagttgtcttggtgggttaacggcaaagaagtacactccggtgtaagcaccgatcctcagccacttaaggaacaacccgcactcaacgactccagatactgtttgagttctaggctgagagtctcagcgacgttttggcagaacccacggaatcatttcagatgtcaggtccagttttacgggttgagcgagaacgacgagtggacacaggatcgggctaaaccagtgacccagattgtatcagccgaagcatggggaagggcagattgcggctttacctcagagtcatatcaacagggcgttttgtctgcaacaatcctttatgagatcctgcttggtaaggcgactctgtatgccgtgcttgttagtgccctggttctgatggcaatggtaaaaagaaaggactctaggggaggttctggcgctactaacttcagcctgctgaagcaggctggagacgtggaggagaaccctggacctatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaacccgtacctcaggaacctacaaatacatctttggaacaggcaccaggctgaaggttttagcaaatatccagaaccctgaccctgcagtatatcagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctg

SEQ ID NO: 108 - DNA, 500 bp, synthetic construct
  source 1..500: mol_type = other DNA
agcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctga

SEQ ID NO: 109 - DNA, 347 bp, synthetic construct; identical to SEQ ID NO: 86
  source 1..347: mol_type = other DNA

SEQ ID NO: 110 - DNA, 116 bp, synthetic construct; identical to SEQ ID NO: 87
  source 1..116: mol_type = other DNA

SEQ ID NO: 111 - DNA, 693 bp, synthetic construct; identical to SEQ ID NO: 78
  source 1..693: mol_type = other DNA

SEQ ID NO: 112 - DNA, 54 bp, synthetic construct; identical to SEQ ID NO: 89
  source 1..54: mol_type = other DNA

SEQ ID NO: 113 - DNA, 939 bp, synthetic construct; identical to SEQ ID NO: 40
  source 1..939: mol_type = other DNA

SEQ ID NO: 114 - DNA, 57 bp, synthetic construct; identical to SEQ ID NO: 91
  source 1..57: mol_type = other DNA

SEQ ID NO: 115 - DNA, 418 bp, synthetic construct; identical to SEQ ID NO: 37
  source 1..418: mol_type = other DNA

SEQ ID NO: 116 - DNA, 500 bp, synthetic construct
  source 1..500: mol_type = other DNA
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggc

SEQ ID NO: 117 - DNA, 2644 bp, synthetic construct
  source 1..2644: mol_type = other DNA
tagaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggatccggcgagggcagaggaagtctgctaacatgcggtgacgtcgaggagaatcctggacctatgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagtccgtggggggcaggcggcacagatacgcagtattttggcccaggcacccggctgacagtgctcgaggaccttaagaatgtgttccctcccgaggtggctgtcttcgaaccaagcgaagccgagatctctcacacacaaaaggctactctcgtgtgtctggcaacgggtttttaccctgatcatgtagagttgtcttggtgggttaacggcaaagaagtacactccggtgtaagcaccgatcctcagccacttaaggaacaacccgcactcaacgactccagatactgtttgagttctaggctgagagtctcagcgacgttttggcagaacccacggaatcatttcagatgtcaggtccagttttacgggttgagcgagaacgacgagtggacacaggatcgggctaaaccagtgacccagattgtatcagccgaagcatggggaagggcagattgcggctttacctcagagtcatatcaacagggcgttttgtctgcaacaatcctttatgagatcctgcttggtaaggcgactctgtatgccgtgcttgttagtgccctggttctgatggcaatggtaaaaagaaaggactctaggggaggttctggcgctactaacttcagcctgctgaagcaggctggagacgtggaggagaaccctggacctatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaacccgtacctcaggaacctacaaatacatctttggaacaggcaccaggctgaaggttttagcaa

SEQ ID NO: 118 - DNA, 3644 bp, synthetic construct
  source 1..3644: mol_type = other DNA
agcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatagaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggatccggcgagggcagaggaagtctgctaacatgcggtgacgtcgaggagaatcctggacctatgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagtccgtggggggcaggcggcacagatacgcagtattttggcccaggcacccggctgacagtgctcgaggaccttaagaatgtgttccctcccgaggtggctgtcttcgaaccaagcgaagccgagatctctcacacacaaaaggctactctcgtgtgtctggcaacgggtttttaccctgatcatgtagagttgtcttggtgggttaacggcaaagaagtacactccggtgtaagcaccgatcctcagccacttaaggaacaacccgcactcaacgactccagatactgtttgagttctaggctgagagtctcagcgacgttttggcagaacccacggaatcatttcagatgtcaggtccagttttacgggttgagcgagaacgacgagtggacacaggatcgggctaaaccagtgacccagattgtatcagccgaagcatggggaagggcagattgcggctttacctcagagtcatatcaacagggcgttttgtctgcaacaatcctttatgagatcctgcttggtaaggcgactctgtatgccgtgcttgttagtgccctggttctgatggcaatggtaaaaagaaaggactctaggggaggttctggcgctactaacttcagcctgctgaagcaggctggagacgtggaggagaaccctggacctatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaacccgtacctcaggaacctacaaatacatctttggaacaggcaccaggctgaaggttttagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggc

SEQ ID NO: 119 - DNA, 346 bp, synthetic construct; identical to SEQ ID NO: 85
  source 1..346: mol_type = other DNA

SEQ ID NO: 120 - DNA, 347 bp, synthetic construct; identical to SEQ ID NO: 86
  source 1..347: mol_type = other DNA

SEQ ID NO: 121 - DNA, 116 bp, synthetic construct; identical to SEQ ID NO: 87
  source 1..116: mol_type = other DNA

SEQ ID NO: 122 - DNA, 693 bp, synthetic construct; identical to SEQ ID NO: 78
  source 1..693: mol_type = other DNA

SEQ ID NO: 123 - DNA, 54 bp, synthetic construct; identical to SEQ ID NO: 89
  source 1..54: mol_type = other DNA

SEQ ID NO: 124 - DNA, 936 bp, synthetic construct; identical to SEQ ID NO: 50
  source 1..936: mol_type = other DNA

SEQ ID NO: 125 - DNA, 57 bp, synthetic construct; identical to SEQ ID NO: 91
  source 1..57: mol_type = other DNA

SEQ ID NO: 126 - DNA, 417 bp, synthetic construct
  source 1..417: mol_type = other DNA
atgaagagcctgcgcgtgctgctggtcatcctgtggctgcaattgagttgggtgtggagccagaaggaccaggtgtttcagcctagcactgtcgcatcatcagagggggccgtcgtggaaatcttttgtaatcacagcgtgtccaacgcctacaatttcttttggtatctgcacttcccaggatgcgcacctaggctgctggtgaagggctctaagcccagccagcagggccggtacaacatgacctatgagcggttcagctcctctctgctgatcctgcaggtgcgggaggcagacgcagccgtgtactattgtgccgtggaggatctgaatcaggccggcacagccctgattttcggcaaaggaacaaccctgagcgtgagcagcaatatccagaaccctgaccctgcagtatat

SEQ ID NO: 127 - DNA, 357 bp, synthetic construct; identical to SEQ ID NO: 93
  source 1..357: mol_type = other DNA

SEQ ID NO: 128 - DNA, 2640 bp, synthetic construct
  source 1..2640: mol_type = other DNA
tagaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggatccggcgagggcagaggaagtctgctaacatgcggtgacgtcgaggagaatcctggacctatgggatcaagactgctgtgctgggtgctgctgtgcctgctgggagccggacctgtgaaagccggggtgactcagactccacgatacctgatcaagaccaggggccagcaggtgacactgtcttgcagcccaatctccggacaccgctccgtgtcttggtaccagcagacccctggacagggactgcagttcctgtttgagtatttctctgagacacagcggaacaagggcaatttccccggccggtttagcggcagacagtttagcaactccaggtctgagatgaatgtgagcaccctggagctgggcgactccgccctgtacctgtgcgcaagctccctggccctgggacaggggaatcagcagtttttcggacccggaacaaggctgaccgtgctggaggaccttaagaatgtgttccctcccgaggtggctgtcttcgaaccaagcgaagccgagatctctcacacacaaaaggctactctcgtgtgtctggcaacgggtttttaccctgatcatgtagagttgtcttggtgggttaacggcaaagaagtacactccggtgtatctaccgatcctcagccacttaaggaacaacccgcactcaacgactccagatactgtttgagttctaggctgagagtctcagcgacgttttggcagaacccacggaatcatttcagatgtcaggtccagttttacgggttgagcgagaacgacgagtggacacaggatcgggctaaaccagtgacccagattgtatcagccgaagcatggggaagggcagattgcggctttacctcagagtcatatcaacagggcgttttgtctgcaacaatcctttatgagatcctgcttggtaaggcgactctgtatgccgtgcttgttagtgccctggttctgatggcaatggtaaaaagaaaggactctaggggaggttctggcgctactaacttcagcctgctgaagcaggctggagacgtggaggagaaccctggacctatgaagagcctgcgcgtgctgctggtcatcctgtggctgcaattgagttgggtgtggagccagaaggaccaggtgtttcagcctagcactgtcgcatcatcagagggggccgtcgtggaaatcttttgtaatcacagcgtgtccaacgcctacaatttcttttggtatctgcacttcccaggatgcgcacctaggctgctggtgaagggctctaagcccagccagcagggccggtacaacatgacctatgagcggttcagctcctctctgctgatcctgcaggtgcgggaggcagacgcagccgtgtactattgtgccgtggaggatctgaatcaggccggcacagccctgattttcggcaaaggaacaaccctgagcgtgagcagcaatatccagaaccctgaccctgcagtatat

SEQ ID NO: 129 - DNA, 3343 bp, synthetic construct
  source 1..3343: mol_type = other DNA
cctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcctagaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggatccggcgagggcagaggaagtctgctaacatgcggtgacgtcgaggagaatcctggacctatgggatcaagactgctgtgctgggtgctgctgtgcctgctgggagccggacctgtgaaagccggggtgactcagactccacgatacctgatcaagaccaggggccagcaggtgacactgtcttgcagcccaatctccggacaccgctccgtgtcttggtaccagcagacccctggacagggactgcagttcctgtttgagtatttctctgagacacagcggaacaagggcaatttccccggccggtttagcggcagacagtttagcaactccaggtctgagatgaatgtgagcaccctggagctgggcgactccgccctgtacctgtgcgcaagctccctggccctgggacaggggaatcagcagtttttcggacccggaacaaggctgaccgtgctggaggaccttaagaatgtgttccctcccgaggtggctgtcttcgaaccaagcgaagccgagatctctcacacacaaaaggctactctcgtgtgtctggcaacgggtttttaccctgatcatgtagagttgtcttggtgggttaacggcaaagaagtacactccggtgtatctaccgatcctcagccacttaaggaacaacccgcactcaacgactccagatactgtttgagttctaggctgagagtctcagcgacgttttggcagaacccacggaatcatttcagatgtcaggtccagttttacgggttgagcgagaacgacgagtggacacaggatcgggctaaaccagtgacccagattgtatcagccgaagcatggggaagggcagattgcggctttacctcagagtcatatcaacagggcgttttgtctgcaacaatcctttatgagatcctgcttggtaaggcgactctgtatgccgtgcttgttagtgccctggttctgatggcaatggtaaaaagaaaggactctaggggaggttctggcgctactaacttcagcctgctgaagcaggctggagacgtggaggagaaccctggacctatgaagagcctgcgcgtgctgctggtcatcctgtggctgcaattgagttgggtgtggagccagaaggaccaggtgtttcagcctagcactgtcgcatcatcagagggggccgtcgtggaaatcttttgtaatcacagcgtgtccaacgcctacaatttcttttggtatctgcacttcccaggatgcgcacctaggctgctggtgaagggctctaagcccagccagcagggccggtacaacatgacctatgagcggttcagctcctctctgctgatcctgcaggtgcgggaggcagacgcagccgtgtactattgtgccgtggaggatctgaatcaggccggcacagccctgattttcggcaaaggaacaaccctgagcgtgagcagcaatatccagaaccctgaccctgcagtatatcagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggc

SEQ ID NO: 130 - DNA, 346 bp, synthetic construct; identical to SEQ ID NO: 85
  source 1..346: mol_type = other DNA

SEQ ID NO: 131 - DNA, 347 bp, synthetic construct; identical to SEQ ID NO: 86
  source 1..347: mol_type = other DNA

SEQ ID NO: 132 - DNA, 116 bp, synthetic construct; identical to SEQ ID NO: 87
  source 1..116: mol_type = other DNA

SEQ ID NO: 133 - DNA, 693 bp, synthetic construct; identical to SEQ ID NO: 78
  source 1..693: mol_type = other DNA

SEQ ID NO: 134 - DNA, 54 bp, synthetic construct; identical to SEQ ID NO: 89
  source 1..54: mol_type = other DNA

SEQ ID NO: 135 - DNA, 936 bp, synthetic construct; identical to SEQ ID NO: 60
  source 1..936: mol_type = other DNA

SEQ ID NO: 136 - DNA, 57 bp, synthetic construct; identical to SEQ ID NO: 91
  source 1..57: mol_type = other DNA

SEQ ID NO: 137 - DNA, 414 bp, synthetic construct
  source 1..414: mol_type = other DNA
atgctactcatcacatcaatgttggtcttatggatgcaattgtcacaggtgaatggacaacaggtaatgcaaattcctcagtaccagcatgtacaagaaggagaggacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcaggtcaaactggggcaaacaacctcttctttgggactggaacgagactcaccgttattccctatatccagaaccctgaccctgcagtatat

SEQ ID NO: 138 - DNA, 357 bp, synthetic construct; identical to SEQ ID NO: 93
  source 1..357: mol_type = other DNA

SEQ ID NO: 139 - DNA, 2637 bp, synthetic construct
  source 1..2637: mol_type = other DNA
tagaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggatccggcgagggcagaggaagtctgctaacatgcggtgacgtcgaggagaatcctggacctatgggctgcaggctgctctgctgtgcggttctctgtctcctgggagcggtccccatggaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaagaagtaggtacagtccccaatcagccccagcattttggtgatgggactcgactctccatcctagaggaccttaataaggtgttccctcccgaggtggctgtcttcgaaccaagcgaagccgagatctctcacacacaaaaggctactctcgtgtgtctggcaacgggttttttccctgatcatgtagagttgtcttggtgggttaacggcaaagaagtacactccggtgtaagcaccgatcctcagccacttaaggaacaacccgcactcaacgactccagatactgtttgagttctaggctgagagtctcagcgacgttttggcagaacccacggaatcatttcagatgtcaggtccagttttacgggttgagcgagaacgacgagtggacacaggatcgggctaaaccagtgacccagattgtatcagccgaagcatggggaagggcagattgcggctttacctcagtttcatatcaacagggcgttttgtctgcaacaatcctttatgagatcctgcttggtaaggcgactctgtatgccgtgcttgttagtgccctggttctgatggcaatggtaaaaagaaaggactttggttctggcgctactaacttcagcctgctgaagcaggctggagacgtggaggagaaccctggacctatgctactcatcacatcaatgttggtcttatggatgcaattgtcacaggtgaatggacaacaggtaatgcaaattcctcagtaccagcatgtacaagaaggagaggacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcaggtcaaactggggcaaacaacctcttctttgggactggaacgagactcaccgttattccctatatccagaaccctgaccctgcagtatat

SEQ ID NO: 140 - DNA, 3340 bp, synthetic construct
  source 1..3340: mol_type = other DNA
cctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcctagaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggatccggcgagggcagaggaagtctgctaacatgcggtgacgtcgaggagaatcctggacctatgggctgcaggctgctctgctgtgcggttctctgtctcctgggagcggtccccatggaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaagaagtaggtacagtccccaatcagccccagcattttggtgatgggactcgactctccatcctagaggaccttaataaggtgttccctcccgaggtggctgtcttcgaaccaagcgaagccgagatctctcacacacaaaaggctactctcgtgtgtctggcaacgggttttttccctgatcatgtagagttgtcttggtgggttaacggcaaagaagtacactccggtgtaagcaccgatcctcagccacttaaggaacaacccgcactcaacgactccagatactgtttgagttctaggctgagagtctcagcgacgttttggcagaacccacggaatcatttcagatgtcaggtccagttttacgggttgagcgagaacgacgagtggacacaggatcgggctaaaccagtgacccagattgtatcagccgaagcatggggaagggcagattgcggctttacctcagtttcatatcaacagggcgttttgtctgcaacaatcctttatgagatcctgcttggtaaggcgactctgtatgccgtgcttgttagtgccctggttctgatggcaatggtaaaaagaaaggactttggttctggcgctactaacttcagcctgctgaagcaggctggagacgtggaggagaaccctggacctatgctactcatcacatcaatgttggtcttatggatgcaattgtcacaggtgaatggacaacaggtaatgcaaattcctcagtaccagcatgtacaagaaggagaggacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcaggtcaaactggggcaaacaacctcttctttgggactggaacgagactcaccgttattccctatatccagaaccctgaccctgcagtatatcagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggc

SEQ ID NO: 141 - DNA, 601 bp, synthetic construct
  source 1..601: mol_type = other DNA
acttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccagga

SEQ ID NO: 142 - DNA, 347 bp, synthetic construct; identical to SEQ ID NO: 86
  source 1..347: mol_type = other DNA

SEQ ID NO: 143 - DNA, 116 bp, synthetic construct
  source 1..116: mol_type = other DNA
gcctggagacgccatccacgctgttttgacttccatagaaggatctcgaggccaccatgcctctgggcctgctgtggctgggcctggccctgctgggcgccctgcacgcccaggcc

SEQ ID NO: 144 - DNA, 1212 bp, synthetic construct; identical to SEQ ID NO: 83
  source 1..1212: mol_type = other DNA

SEQ ID NO: 145 - DNA, 57 bp, synthetic construct
  source 1..57: mol_type = other DNA
gccaccaactttagtctgctcaagcaagccggggacgtcgaggaaaatcctgggcca

SEQ ID NO: 146 - DNA, 267 bp, synthetic construct; identical to SEQ ID NO: 84
  source 1..267: mol_type = other DNA

SEQ ID NO: 147 - DNA, 57 bp, synthetic construct
  source 1..57: mol_type = other DNA
gccacaaatttctccctgctgaaacaggccggcgacgtggaagagaatcccggacct

SEQ ID NO: 148 - DNA, 66 bp, synthetic construct
  source 1..66: mol_type = other DNA
atgcccaatcctagacctggcaagcccagcgctccttctcttgctcttggaccttctcctggtgca

SEQ ID NO: 149 - DNA, 600 bp, synthetic construct
  source 1..600: mol_type = other DNA
tcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtg

SEQ ID NO: 150 - DNA, 2141 bp, synthetic construct
  source 1..2141: mol_type = other DNA
tgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctctgggcctgctgtggctgggcctggccctgctgggcgccctgcacgcccaggccgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatccggcgccaccaactttagtctgctcaagcaagccggggacgtcgaggaaaatcctgggccagaaatgtggcacgaaggactcgaggaagccagtcggctgtattttggcgagcggaatgtgaaagggatgtttgaagtgctcgagcctctccacgctatgatggaacggggaccccagactctcaaagaaaccagctttaatcaggcttacggacgcgacctcatggaagctcaagaatggtgtagaaagtatatgaagagtggcaacgtgaaagatctgacacaagcctgggatctctattatcacgtgttcagacgcatcagcaaaggcagcggcgccacaaatttctccctgctgaaacaggccggcgacgtggaagagaatcccggacctatgcccaatcctagacctggcaagcccagcgctccttctcttgctcttggaccttctcctggtgca

SEQ ID NO: 151 - DNA, 3342 bp, synthetic construct
  source 1..3342: mol_type = other DNA
acttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggatgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctctgggcctgctgtggctgggcctggccctgctgggcgccctgcacgcccaggccgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatccggcgccaccaactttagtctgctcaagcaagccggggacgtcgaggaaaatcctgggccagaaatgtggcacgaaggactcgaggaagccagtcggctgtattttggcgagcggaatgtgaaagggatgtttgaagtgctcgagcctctccacgctatgatggaacggggaccccagactctcaaagaaaccagctttaatcaggcttacggacgcgacctcatggaagctcaagaatggtgtagaaagtatatgaagagtggcaacgtgaaagatctgacacaagcctgggatctctattatcacgtgttcagacgcatcagcaaaggcagcggcgccacaaatttctccctgctgaaacaggccggcgacgtggaagagaatcccggacctatgcccaatcctagacctggcaagcccagcgctccttctcttgctcttggaccttctcctggtgcatcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtg

SEQ ID NO: 152 - DNA, 650 bp, synthetic construct
  source 1..650: mol_type = other DNA
cccactacatccaagctgctagcactgctcctgatccagcttcagattaagtctcagaatctacccacttctcgccttctccactgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggac

SEQ ID NO: 153 - DNA, 347 bp, synthetic construct; identical to SEQ ID NO: 86
  source 1..347: mol_type = other DNA

SEQ ID NO: 154 - DNA, 116 bp, synthetic construct; identical to SEQ ID NO: 143
  source 1..116: mol_type = other DNA

SEQ ID NO: 155 - DNA, 1212 bp, synthetic construct; identical to SEQ ID NO: 83
  source 1..1212: mol_type = other DNA

SEQ ID NO: 156 - DNA, 57 bp, synthetic construct; identical to SEQ ID NO: 145
  source 1..57: mol_type = other DNA

SEQ ID NO: 157 - DNA, 267 bp, synthetic construct; identical to SEQ ID NO: 84
  source 1..267: mol_type = other DNA

SEQ ID NO: 158 - DNA, 57 bp, synthetic construct; identical to SEQ ID NO: 147
  source 1..57: mol_type = other DNA

SEQ ID NO: 159 - DNA, 12 bp, synthetic construct
  source 1..12: mol_type = other DNA
atgccaaatcct

SEQ ID NO: 160 - DNA, 650 bp, synthetic construct
  source 1..650: mol_type = other DNA
aggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgc

SEQ ID NO: 161 - DNA, 2093 bp, synthetic construct
  source 1..2093: mol_type = other DNA
acgatgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctctgggcctgctgtggctgggcctggccctgctgggcgccctgcacgcccaggccgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatccggcgccaccaactttagtctgctcaagcaagccggggacgtcgaggaaaatcctgggccagaaatgtggcacgaaggactcgaggaagccagtcggctgtattttggcgagcggaatgtgaaagggatgtttgaagtgctcgagcctctccacgctatgatggaacggggaccccagactctcaaagaaaccagctttaatcaggcttacggacgcgacctcatggaagctcaagaatggtgtagaaagtatatgaagagtggcaacgtgaaagatctgacacaagcctgggatctctattatcacgtgttcagacgcatcagcaaaggcagcggcgccacaaatttctccctgctgaaacaggccggcgacgtggaagagaatcccggacctatgccaaatcct

SEQ ID NO: 162 - DNA, 3393 bp, synthetic construct
  source 1..3393: mol_type = other DNA
cccactacatccaagctgctagcactgctcctgatccagcttcagattaagtctcagaatctacccacttctcgccttctccactgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacacgatgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctctgggcctgctgtggctgggcctggccctgctgggcgccctgcacgcccaggccgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatccggcgccaccaactttagtctgctcaagcaagccggggacgtcgaggaaaatcctgggccagaaatgtggcacgaaggactcgaggaagccagtcggctgtattttggcgagcggaatgtgaaagggatgtttgaagtgctcgagcctctccacgctatgatggaacggggaccccagactctcaaagaaaccagctttaatcaggcttacggacgcgacctcatggaagctcaagaatggtgtagaaagtatatgaagagtggcaacgtgaaagatctgacacaagcctgggatctctattatcacgtgttcagacgcatcagcaaaggcagcggcgccacaaatttctccctgctgaaacaggccggcgacgtggaagagaatcccggacctatgccaaatcctaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgc

SEQ ID NO: 163 - DNA, 651 bp, synthetic construct
  source 1..651: mol_type = other DNA
actgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggag

SEQ ID NO: 164 - DNA, 347 bp, synthetic construct; identical to SEQ ID NO: 86
  source 1..347: mol_type = other DNA

SEQ ID NO: 165 - DNA, 116 bp, synthetic construct; identical to SEQ ID NO: 143
  source 1..116: mol_type = other DNA

SEQ ID NO: 166 - DNA, 1212 bp, synthetic construct; identical to SEQ ID NO: 83
  source 1..1212: mol_type = other DNA

SEQ ID NO: 167 - DNA, 57 bp, synthetic construct; identical to SEQ ID NO: 145
  source 1..57: mol_type = other DNA

SEQ ID NO: 168 - DNA, 267 bp, synthetic construct; identical to SEQ ID NO: 84
  source 1..267: mol_type = other DNA

SEQ ID NO: 169 - DNA, 57 bp, synthetic construct; identical to SEQ ID NO: 147
  source 1..57: mol_type = other DNA

SEQ ID NO: 170 - DNA, 96 bp, synthetic construct
  source 1..96: mol_type = other DNA
atgcccaatcctagacctggcaagcccagcgctccttctcttgctcttggaccttctcctggtgcaagtccatcttggcgagcagctccaaaggct

SEQ ID NO: 171 - DNA, 650 bp, synthetic construct
  source 1..650: mol_type = other DNA
tcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccaca

SEQ ID NO: 172 - DNA, 2178 bp, synthetic construct
  source 1..2178: mol_type = other DNA
agctgcatgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctctgggcctgctgtggctgggcctggccctgctgggcgccctgcacgcccaggccgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatccggcgccaccaactttagtctgctcaagcaagccggggacgtcgaggaaaatcctgggccagaaatgtggcacgaaggactcgaggaagccagtcggctgtattttggcgagcggaatgtgaaagggatgtttgaagtgctcgagcctctccacgctatgatggaacggggaccccagactctcaaagaaaccagctttaatcaggcttacggacgcgacctcatggaagctcaagaatggtgtagaaagtatatgaagagtggcaacgtgaaagatctgacacaagcctgggatctctattatcacgtgttcagacgcatcagcaaaggcagcggcgccacaaatttctccctgctgaaacaggccggcgacgtggaagagaatcccggacctatgcccaatcctagacctggcaagcccagcgctccttctcttgctcttggaccttctcctggtgcaagtccatcttggcgagcagctccaaaggct

SEQ ID NO: 173 - DNA, 3479 bp, synthetic construct
  source 1..3479: mol_type = other DNA
actgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagagctgcatgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctctgggcctgctgtggctgggcctggccctgctgggcgccctgcacgcccaggccgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatccggcgccaccaactttagtctgctcaagcaagccggggacgtcgaggaaaatcctgggccagaaatgtggcacgaaggactcgaggaagccagtcggctgtattttggcgagcggaatgtgaaagggatgtttgaagtgctcgagcctctccacgctatgatggaacggggaccccagactctcaaagaaaccagctttaatcaggcttacggacgcgacctcatggaagctcaagaatggtgtagaaagtatatgaagagtggcaacgtgaaagatctgacacaagcctgggatctctattatcacgtgttcagacgcatcagcaaaggcagcggcgccacaaatttctccctgctgaaacaggccggcgacgtggaagagaatcccggacctatgcccaatcctagacctggcaagcccagcgctccttctcttgctcttggaccttctcctggtgcaagtccatcttggcgagcagctccaaaggcttcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccaca

SEQ ID NO: 174 - DNA, 657 bp, synthetic construct
  source 1..657: mol_type = other DNA
acttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagggctgcacccaaagcctcagacctgctgggggcccgggg

SEQ ID NO: 175 - DNA, 17 bp, synthetic construct
  source 1..17: mol_type = other DNA
accaggtggaacattct

SEQ ID NO: 176 - DNA, 347 bp, synthetic construct; identical to SEQ ID NO: 86
  source 1..347: mol_type = other DNA

SEQ ID NO: 177 - DNA, 116 bp, synthetic construct; identical to SEQ ID NO: 143
  source 1..116: mol_type = other DNA

SEQ ID NO: 178 - DNA, 1212 bp, synthetic construct; identical to SEQ ID NO: 83
  source 1..1212: mol_type = other DNA

SEQ ID NO: 179 - DNA, 57 bp, synthetic construct; identical to SEQ ID NO: 145
  source 1..57: mol_type = other DNA

SEQ ID NO: 180 - DNA, 267 bp, synthetic construct; identical to SEQ ID NO: 84
  source 1..267: mol_type = other DNA

SEQ ID NO: 181 - DNA, 57 bp, synthetic construct; identical to SEQ ID NO: 147
  source 1..57: mol_type = other DNA

SEQ ID NO: 182 - DNA, 141 bp, synthetic construct
  source 1..141: mol_type = other DNA
atgcccaatcctagacctggcaagcccagcgctccttctcttgctcttggaccttctcctggtgcaagtccatcttggcgagcagctccaaaggctagtgatcttctcggagctagaggtcctggaggcacatttcaggga

SEQ ID NO: 183 - DNA, 651 bp, synthetic construct
  source 1..651: mol_type = other DNA
cgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggcccct

SEQ ID NO: 184 - DNA, 2232 bp, synthetic construct
  source 1..2232: mol_type = other DNA
accaggtggaacattctgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctctgggcctgctgtggctgggcctggccctgctgggcgccctgcacgcccaggccgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatccggcgccaccaactttagtctgctcaagcaagccggggacgtcgaggaaaatcctgggccagaaatgtggcacgaaggactcgaggaagccagtcggctgtattttggcgagcggaatgtgaaagggatgtttgaagtgctcgagcctctccacgctatgatggaacggggaccccagactctcaaagaaaccagctttaatcaggcttacggacgcgacctcatggaagctcaagaatggtgtagaaagtatatgaagagtggcaacgtgaaagatctgacacaagcctgggatctctattatcacgtgttcagacgcatcagcaaaggcagcggcgccacaaatttctccctgctgaaacaggccggcgacgtggaagagaatcccggacctatgcccaatcctagacctggcaagcccagcgctccttctcttgctcttggaccttctcctggtgcaagtccatcttggcgagcagctccaaaggctagtgatcttctcggagctagaggtcctggaggcacatttcaggga

SEQ ID NO: 185 - DNA, 3540 bp, synthetic construct
  source 1..3540: mol_type = other DNA
acttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagggctgcacccaaagcctcagacctgctgggggcccggggaccaggtggaacattctgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctctgggcctgctgtggctgggcctggccctgctgggcgccctgcacgcccaggccgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatccggcgccaccaactttagtctgctcaagcaagccggggacgtcgaggaaaatcctgggccagaaatgtggcacgaaggactcgaggaagccagtcggctgtattttggcgagcggaatgtgaaagggatgtttgaagtgctcgagcctctccacgctatgatggaacggggaccccagactctcaaagaaaccagctttaatcaggcttacggacgcgacctcatggaagctcaagaatggtgtagaaagtatatgaagagtggcaacgtgaaagatctgacacaagcctgggatctctattatcacgtgttcagacgcatcagcaaaggcagcggcgccacaaatttctccctgctgaaacaggccggcgacgtggaagagaatcccggacctatgcccaatcctagacctggcaagcccagcgctccttctcttgctcttggaccttctcctggtgcaagtccatcttggcgagcagctccaaaggctagtgatcttctcggagctagaggtcctggaggcacatttcagggacgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggcccct

SEQ ID NO: 186 - DNA, 650 bp, synthetic construct
  source 1..650: mol_type = other DNA
gccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggc

SEQ ID NO: 187 - DNA, 347 bp, synthetic construct; identical to SEQ ID NO: 86
  source 1..347: mol_type = other DNA

SEQ ID NO: 188 - DNA, 116 bp, synthetic construct; identical to SEQ ID NO: 143
  source 1..116: mol_type = other DNA

SEQ ID NO: 189 - DNA, 1212 bp, synthetic construct; identical to SEQ ID NO: 83
  source 1..1212: mol_type = other DNA

SEQ ID NO: 190 - DNA, 57 bp, synthetic construct; identical to SEQ ID NO: 145
  source 1..57: mol_type = other DNA

SEQ ID NO: 191 - DNA, 267 bp, synthetic construct; identical to SEQ ID NO: 84
  source 1..267: mol_type = other DNA

SEQ ID NO: 192 - DNA, 57 bp, synthetic construct; identical to SEQ ID NO: 147
  source 1..57: mol_type = other DNA

SEQ ID NO: 193 - DNA, 168 bp, synthetic construct
  source 1..168: mol_type = other DNA
atgcccaatcctagacctggcaagcccagcgctccttctcttgctcttggaccttctcctggtgcaagtccatcttggcgagcagctccaaaggctagtgatcttctcggagctagaggtcctggaggcacatttcaaggtagagacttgaggggaggtgctcacgct

SEQ ID NO: 194 - DNA, 650 bp, synthetic construct
  source 1..650: mol_type = other DNA
tcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccag

SEQ ID NO: 195 - DNA, 2250 bp, synthetic construct
  source 1..2250: mol_type = other DNA
tcatgcatgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctctgggcctgctgtggctgggcctggccctgctgggcgccctgcacgcccaggccgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatccggcgccaccaactttagtctgctcaagcaagccggggacgtcgaggaaaatcctgggccagaaatgtggcacgaaggactcgaggaagccagtcggctgtattttggcgagcggaatgtgaaagggatgtttgaagtgctcgagcctctccacgctatgatggaacggggaccccagactctcaaagaaaccagctttaatcaggcttacggacgcgacctcatggaagctcaagaatggtgtagaaagtatatgaagagtggcaacgtgaaagatctgacacaagcctgggatctctattatcacgtgttcagacgcatcagcaaaggcagcggcgccacaaatttctccctgctgaaacaggccggcgacgtggaagagaatcccggacctatgcccaatcctagacctggcaagcccagcgctccttctcttgctcttggaccttctcctggtgcaagtccatcttggcgagcagctccaaaggctagtgatcttctcggagctagaggtcctggaggcacatttcaaggtagagacttgaggggaggtgctcacgct

SEQ ID NO: 196 - DNA, 3550 bp, synthetic construct
  source 1..3550: mol_type = other DNA
gccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggctcatgcatgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctctgggcctgctgtggctgggcctggccctgctgggcgccctgcacgcccaggccgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatccggcgccaccaactttagtctgctcaagcaagccggggacgtcgaggaaaatcctgggccagaaatgtggcacgaaggactcgaggaagccagtcggctgtattttggcgagcggaatgtgaaagggatgtttgaagtgctcgagcctctccacgctatgatggaacggggaccccagactctcaaagaaaccagctttaatcaggcttacggacgcgacctcatggaagctcaagaatggtgtagaaagtatatgaagagtggcaacgtgaaagatctgacacaagcctgggatctctattatcacgtgttcagacgcatcagcaaaggcagcggcgccacaaatttctccctgctgaaacaggccggcgacgtggaagagaatcccggacctatgcccaatcctagacctggcaagcccagcgctccttctcttgctcttggaccttctcctggtgcaagtccatcttggcgagcagctccaaaggctagtgatcttctcggagctagaggtcctggaggcacatttcaaggtagagacttgaggggaggtgctcacgcttcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccag

SEQ ID NO: 197 - DNA, 650 bp, synthetic construct
  source 1..650: mol_type = other DNA
cactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatctt

SEQ ID NO: 198 - DNA, 347 bp, synthetic construct; identical to SEQ ID NO: 86
  source 1..347: mol_type = other DNA

SEQ ID NO: 199 - DNA, 116 bp, synthetic construct; identical to SEQ ID NO: 143
  source 1..116: mol_type = other DNA

SEQ ID NO: 200 - DNA, 1212 bp, synthetic construct; identical to SEQ ID NO: 83
  source 1..1212: mol_type = other DNA

SEQ ID NO: 201 - DNA, 57 bp, synthetic construct; identical to SEQ ID NO: 145
  source 1..57: mol_type = other DNA

SEQ ID NO: 202 - DNA, 267 bp, synthetic construct; identical to SEQ ID NO: 84
  source 1..267: mol_type = other DNA

SEQ ID NO: 203 - DNA, 57 bp, synthetic construct; identical to SEQ ID NO: 147
  source 1..57: mol_type = other DNA

SEQ ID NO: 204 - DNA, 159 bp, synthetic construct
  source 1..159: mol_type = other DNA
atgcccaatcctagacctggcaagcccagcgctccttctcttgctcttggaccttctcctggtgcaagtccatcttggcgagcagctccaaaggctagtgatcttctcggagctagaggtcctggaggcacatttcaaggtagggacttgcgtggaggt

SEQ ID NO: 205 - DNA, 650 bp, synthetic construct
  source 1..650: mol_type = other DNA
gcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggca

SEQ ID NO: 206 - DNA, 2237 bp, synthetic construct
  source 1..2237: mol_type = other DNA
aggtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctctgggcctgctgtggctgggcctggccctgctgggcgccctgcacgcccaggccgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatccggcgccaccaactttagtctgctcaagcaagccggggacgtcgaggaaaatcctgggccagaaatgtggcacgaaggactcgaggaagccagtcggctgtattttggcgagcggaatgtgaaagggatgtttgaagtgctcgagcctctccacgctatgatggaacggggaccccagactctcaaagaaaccagctttaatcaggcttacggacgcgacctcatggaagctcaagaatggtgtagaaagtatatgaagagtggcaacgtgaaagatctgacacaagcctgggatctctattatcacgtgttcagacgcatcagcaaaggcagcggcgccacaaatttctccctgctgaaacaggccggcgacgtggaagagaatcccggacctatgcccaatcctagacctggcaagcccagcgctccttctcttgctcttggaccttctcctggtgcaagtccatcttggcgagcagctccaaaggctagtgatcttctcggagctagaggtcctggaggcacatttcaaggtagggacttgcgtggaggt

SEQ ID NO: 207 - DNA, 3537 bp, synthetic construct
  source 1..3537: mol_type = other DNA
cactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttaggtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctctgggcctgctgtggctgggcctggccctgctgggcgccctgcacgcccaggccgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatccggcgccaccaactttagtctgctcaagcaagccggggacgtcgaggaaaatcctgggccagaaatgtggcacgaaggactcgaggaagccagtcggctgtattttggcgagcggaatgtgaaagggatgtttgaagtgctcgagcctctccacgctatgatggaacggggaccccagactctcaaagaaaccagctttaatcaggcttacggacgcgacctcatggaagctcaagaatggtgtagaaagtatatgaagagtggcaacgtgaaagatctgacacaagcctgggatctctattatcacgtgttcagacgcatcagcaaaggcagcggcgccacaaatttctccctgctgaaacaggccggcgacgtggaagagaatcccggacctatgcccaatcctagacctggcaagcccagcgctccttctcttgctcttggaccttctcctggtgcaagtccatcttggcgagcagctccaaaggctagtgatcttctcggagctagaggtcctggaggcacatttcaaggtagggacttgcgtggaggtgcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggca

SEQ ID NO: 208 - DNA, 657 bp, synthetic construct; identical to SEQ ID NO: 174
  source 1..657: mol_type = other DNA

SEQ ID NO: 209 - DNA, 20 bp, synthetic construct
  source 1..20: mol_type = other DNA
cccagggggaaccttccagt

SEQ ID NO: 210 - DNA, 347 bp, synthetic construct; identical to SEQ ID NO: 86
  source 1..347: mol_type = other DNA

SEQ ID NO: 211 - DNA, 116 bp, synthetic construct; identical to SEQ ID NO: 87
  source 1..116: mol_type = other DNA

SEQ ID NO: 212 - DNA, 1212 bp, synthetic construct; identical to SEQ ID NO: 83
  source 1..1212: mol_type = other DNA

SEQ ID NO: 213 - DNA, 57 bp, synthetic construct; identical to SEQ ID NO: 145
  source 1..57: mol_type = other DNA

SEQ ID NO: 214 - DNA, 267 bp, synthetic construct; identical to SEQ ID NO: 84
  source 1..267: mol_type = other DNA

SEQ ID NO: 215 - DNA, 57 bp, synthetic construct; identical to SEQ ID NO: 147
  source 1..57: mol_type = other DNA

SEQ ID NO: 216 - DNA, 138 bp, synthetic construct
  source 1..138: mol_type = other DNA
atgcccaatcctagacctggcaagcccagcgctccttctcttgctcttggaccttctcctggtgcaagtccatcttggcgagcagctccaaaggctagtgatcttctcggagctagaggtcctggaggcacatttgga

SEQ ID NO: 217 - DNA, 651 bp, synthetic construct; identical to SEQ ID NO: 183
  source 1..651: mol_type = other DNA

SEQ ID NO: 218 - DNA, 2232 bp, synthetic construct
  source 1..2232: mol_type = other DNA
cccagggggaaccttccagtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgccgccaccatgcctctgggcctgctgtggctgggcctggccctgctgggcgccctgcacgcccaggccgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatccggcgccaccaactttagtctgctcaagcaagccggggacgtcgaggaaaatcctgggccagaaatgtggcacgaaggactcgaggaagccagtcggctgtattttggcgagcggaatgtgaaagggatgtttgaagtgctcgagcctctccacgctatgatggaacggggaccccagactctcaaagaaaccagctttaatcaggcttacggacgcgacctcatggaagctcaagaatggtgtagaaagtatatgaagagtggcaacgtgaaagatctgacacaagcctgggatctctattatcacgtgttcagacgcatcagcaaaggcagcggcgccacaaatttctccctgctgaaacaggccggcgacgtggaagagaatcccggacctatgcccaatcctagacctggcaagcccagcgctccttctcttgctcttggaccttctcctggtgcaagtccatcttggcgagcagctccaaaggctagtgatcttctcggagctagaggtcctggaggcacatttgga

SEQ ID NO: 219 - DNA, 3540 bp, synthetic construct
  source 1..3540: mol_type = other DNA
acttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgccgccaccatgcctctgggcctgctgtggctgggcctggccctgctgggcgccctgcacgcccaggccgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgacgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagtttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcatgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccaccaactccaggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatccggcgccaccaactttagtctgctcaagcaagccggggacgtcgaggaaaatcctgggccagaaatgtggcacgaaggactcgaggaagccagtcggctgtattttggcgagcggaatgtgaaagggatgtttgaagtgctcgagcctctccacgctatgatggaacggggaccccagactctcaaagaaaccagctttaatcaggcttacggacgcgacctcatggaagctcaagaatggtgtagaaagtatatgaagagtggcaacgtgaaagatctgacacaagcctgggatctctattatcacgtgttcagacgcatcagcaaaggcagcggcgccacaaatttctccctgctgaaacaggccggcgacgtggaagagaatcccggacctatgcccaatcctagacctggcaagcccagcgctccttctcttgctcttggaccttctcctggtgcaagtccatcttggcgagcagctccaaaggctagtgatcttctcggagctagaggtcctggaggcacatttggacgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggcccct

SEQ ID NO: 220 - DNA, 347 bp, synthetic construct; identical to SEQ ID NO: 86
  source 1..347: mol_type = other DNA

SEQ ID NO: 221 - DNA, 54 bp, synthetic construct; identical to SEQ ID NO: 89
  source 1..54: mol_type = other DNA

SEQ ID NO: 222 - protein, 18 aa, synthetic construct
EGRGSLLTCGDVEENPGP

SEQ ID NO: 223 - DNA, 57 bp, synthetic construct; identical to SEQ ID NO: 91
  source 1..57: mol_type = other DNA

SEQ ID NO: 224 - DNA, 57 bp, synthetic construct; identical to SEQ ID NO: 145
  source 1..57: mol_type = other DNA

SEQ ID NO: 225 - DNA, 57 bp, synthetic construct; identical to SEQ ID NO: 147
  source 1..57: mol_type = other DNA

SEQ ID NO: 226 - protein, 19 aa, synthetic construct
ATNFSLLKQAGDVEENPGP

SEQ ID NO: 227 - protein, 19 aa, synthetic construct
ATNFSLLKQAGDVEENPGP

SEQ ID NO: 228 - protein, 19 aa, synthetic construct
ATNFSLLKQAGDVEENPGP

SEQ ID NO: 229 - protein, 4 aa, synthetic construct
GGGS

SEQ ID NO: 230 - protein, 7 aa, synthetic construct
GGGSGGG

SEQ ID NO: 231 - protein, 20 aa, synthetic construct
QLYHFLQIPTHEEHLFYVLS

SEQ ID NO: 232 - protein, 20 aa, synthetic construct
KWCANPDWIHIDTTPFAGLV

SEQ ID NO: 233 - protein, 20 aa, synthetic construct
KVNFFRMVISNPAATHQDID

SEQ ID NO: 234 - protein, 20 aa, synthetic construct
KGMAALPRLIAFTSEHSHFS

SEQ ID NO: 235 - protein, 15 aa, synthetic construct
SLQPLALEGSLQKRG

SEQ ID NO: 236 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 237 - RNA, 100 nt, synthetic construct
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 238 - RNA, 135 nt, synthetic construct
  source 1..135: mol_type = other RNA
nnnnnnnnnnnnnnnnnnnnnnnnngtcatagttccattaaagccaaaagtggctttgatgtttctatgataagggtttcgacccgtggcgtcggggatcgcctgcccattgaaatgggcttctccccatttatt

SEQ ID NO: 239 - RNA, 135 nt, synthetic construct
  source 1..135: mol_type = other RNA
nnnnnnnnnnnnnnnnnnnnnnnnngtcatagttccatgaaagccaaaagtggctttgatgtttctatgataagggtttcggcccgtggcgtcggggatcgcctgcccattccgatgggcttctccccatttatt

SEQ ID NO: 240 - RNA, 20 nt, synthetic construct
  source 1..20: mol_type = other RNA
tctctcagctggtacacggc

SEQ ID NO: 241 - RNA, 20 nt, synthetic construct
  source 1..20: mol_type = other RNA
tccagctgggcgaggctcct

SEQ ID NO: 242 - RNA, 20 nt, synthetic construct
  source 1..20: mol_type = other RNA
tcgaagatctcggccctgga

SEQ ID NO: 243 - RNA, 20 nt, synthetic construct
  source 1..20: mol_type = other RNA
cgcctcgaagatctcggccc

SEQ ID NO: 244 - RNA, 20 nt, synthetic construct
  source 1..20: mol_type = other RNA
gggccgagatcttcgaggcg

SEQ ID NO: 245 - protein, 20 aa, synthetic construct
QLYAFLQIPTHEEHLFYVLS

SEQ ID NO: 246 - protein, 20 aa, synthetic construct
QLYHALQIPTHEEHLFYVLS

SEQ ID NO: 247 - protein, 20 aa, synthetic construct
QLYHFAQIPTHEEHLFYVLS

SEQ ID NO: 248 - protein, 20 aa, synthetic construct
QLYHFLAIPTHEEHLFYVLS

SEQ ID NO: 249 - protein, 20 aa, synthetic construct
QLYHFLQAPTHEEHLFYVLS

SEQ ID NO: 250 - protein, 20 aa, synthetic construct
QLYHFLQIATHEEHLFYVLS

SEQ ID NO: 251 - protein, 20 aa, synthetic construct
QLYHFLQIPAHEEHLFYVLS

SEQ ID NO: 252 - protein, 20 aa, synthetic construct
QLYHFLQIPTAEEHLFYVLS

SEQ ID NO: 253 - protein, 20 aa, synthetic construct
QLYHFLQIPTHAEHLFYVLS

SEQ ID NO: 254 - protein, 20 aa, synthetic construct
QLYHFLQIPTHEAHLFYVLS

SEQ ID NO: 255 - protein, 20 aa, synthetic construct
QLYHFLQIPTHEEALFYVLS

SEQ ID NO: 256 - protein, 15 aa, Legionella sp.
SDSVLQIAAHEEHFG

SEQ ID NO: 257 - protein, 15 aa, unidentified
  source 1..15: note = Bacillus toyonensis
YDEVLQIPTHQENTQ

SEQ ID NO: 258 - protein, 15 aa, Treponema sp.
AASFLHIPYFTHECD

SEQ ID NO: 259 - protein, 15 aa, Treponema sp.
AARFLHIPYYTHECD

SEQ ID NO: 260 - protein, 15 aa, Treponema sp.
AAKFLHIPVFTHECD

SEQ ID NO: 261 - protein, 15 aa, unidentified
  source 1..15: note = Chlamydia caviae
CSPFLQIPSYEPIEY

SEQ ID NO: 262 - protein, 15 aa, unidentified
  source 1..15: note = Pseudogymnoascus sp. VKM F-4514 (FW-929)
SAMFLDIPTHPEEHS

SEQ ID NO: 263 - protein, 15 aa, unidentified
  source 1..15: note = Blastomyces parvus
KYEFLPIPTYEEATS

SEQ ID NO: 264 - protein, 15 aa, unidentified
  source 1..15: note = Pneumocystis jirovecii RU7
FTDFLNIPSYEELND

SEQ ID NO: 265 - protein, 15 aa, unidentified
  source 1..15: note = Encephalitozoon hellem
LSEFLKIPNHEITLL

SEQ ID NO: 266 - skipped

SEQ ID NO: 267 - protein, 15 aa, unidentified
  source 1..15: note = Encephalitozoon cuniculi GB-M1
QQKFLKNIDTHEEAE

SEQ ID NO: 268 - protein, 15 aa, unidentified
  source 1..15: note = Toxoplasma gondii ARI
ESLFLQLSTHTEASA

SEQ ID NO: 269 - protein, 15 aa, Legionella lansingensis
DQLFLALPAHEERIS

(19) United States
(12) Patent Application Publication
(10) Pub. No.: US 2025/0213686 A1
(43) Pub. Date: Jul. 3, 2025

(54) COMPOSITIONS AND METHODS FOR ENGINEERING TREG CELLS FOR TREATMENT OF DIABETES

(71) Applicants: Seattle Children's Hospital (dba Seattle Children's Research Institute), Seattle, WA (US); Benaroya Research Institute at Virginia Mason, Seattle, WA (US); Gentibio, Inc., Cambridge, MA (US)

(72) Inventors: Jane Buckner, Seattle, WA (US); David J. Rawlings, Seattle, WA (US); Peter J. Cook, Seattle, WA (US); Soo Jung Yang, Seattle, WA (US); Tom Wickham, Cambridge, MA (US); Chandra Patel, Cambridge, MA (US); Gene Uenishi, Cambridge, MA (US); Philippe Kieffer-Kwon, Cambridge, MA (US)

(21) Appl. No.: 18/719,083

(22) PCT Filed: Dec. 19, 2022
(86) PCT No.: PCT/US2022/081929

§ 371 Date: Jun. 12, 2024

Related U.S. Application Data
(60) Provisional application No. 63/292,125, filed on Dec. 21, 2021.
(60) Provisional application No. 63/363,918, filed on Apr. 29, 2022.
(60) Provisional application No. 63/364,285, filed on May 6, 2022.
(60) Provisional application No. 63/378,928, filed on Oct. 10, 2022.
(60) Provisional application No. 63/384,830, filed on Nov. 23, 2022.

(51) Int. Cl. A61K 40/11; A61K 31/436; A61K 40/32; A61K 40/41; A61P 3/10; C07K 14/715; C07K 14/725; C12N 5/0783; C12N 9/22; C12N 9/90; C12N 15/11; C12N 15/90

(52) U.S. Cl. CPC A61K 40/11; A61K 31/436; A61K 40/32; A61K 40/416; A61P 3/10; C07K 14/7051; C07K 14/7155; C12N 5/0637; C12N 9/22; C12N 9/90; C12N 15/11; C12N 15/907; C12Y 502/01008; C12N 2310/20; C12N 2510/00

(57) ABSTRACT

Described herein are compositions and methods for engineering Treg cells for treatment of diabetes. The engineered Treg cells provided herein may be dual-edited (i.e., edited in two different loci in the cell genome), a first locus being the FOXP3 locus and the second locus being the TRAC locus. The engineering of dual-edited Treg cells as provided here may include selective expansion of dual-edited cells using a ligand that initiates and/or maintains IL-2 signal transduction in dual-edited cells. The engineering of dual-edited Treg cells as provided here may stably express FoxP3 and an exogenous TCR.

### RELATED APPLICATIONS

This application claims priority to U.S. Provisional Application No. 63/292,125, filed Dec. 21, 2021; U.S. Provisional Application No. 63/363,918, filed Apr. 29, 2022; U.S. Provisional Application No. 63/364,285, filed May 6, 2022; U.S. Provisional Application No. 63/378,928, filed Oct. 10, 2022; and U.S. Provisional Application No. 63/384,830, filed Nov. 23, 2022, the contents of each of which are incorporated by reference herein in their entirety.

### REFERENCE TO AN ELECTRONIC SEQUENCE LISTING

The contents of the electronic Sequence Listing (G097170024WO-SEQ-NTJ.xml; Size: 365,549 bytes; and Date of Creation: Dec. 8, 2022) are herein incorporated by reference in their entirety.

### BACKGROUND

Type 1 diabetes (T1D), also referred to as juvenile diabetes or insulin-dependent diabetes, is a chronic condition in which the pancreas produces little or no insulin. Cellular therapies using regulatory T cells (Tregs) may be useful to treat numerous types of autoimmune diseases, including T1D.

### SUMMARY

Provided herein are genetically modified engineered regulatory T (EngTreg) cells for treatment of type 1 diabetes (T1D), comprising two inserted nucleic acids comprising: a first nucleic acid inserted into the TRAC locus and a second nucleic acid inserted into the FOXP3 locus, and methods and systems for making the same. T1D accounts for 5% to 10% of diabetes cases worldwide and has no cure. T1D can occur at any age, but the average age at diagnosis is 8 years old, with males displaying a higher prevalence after puberty. Globally, the incidence of T1D has increased 3% to 4% annually, and from 2001 to 2009 there was a ˜20% increase in T1D among persons aged 0 to 19 years. T1D is a chronic autoimmune disease caused by T-lymphocyte-mediated destruction of insulin-producing beta cells, characterized by a pre-symptomatic period of variable length that eventually leads to insulin deficiency with hyperglycaemia. Poorly controlled hyperglycaemia can result in systemic multiorgan damage, which is often irreversible.

Lifelong exogenous insulin administration is required to control hyperglycaemia and associated clinical signs and symptoms. Despite recent advances in continuous blood glucose monitoring and automated insulin administration to maximize the time in (normoglycemic) range (TIR), achieving long-term normoglycemic control is a therapeutically challenging goal. Poor glycaemic control is associated with significant sequalae and reduced quality of life, mediated by micro- and macrovascular complications. In addition to complications of the disease itself, patients and their families must contend with potentially life-threatening major hypoglycaemic episodes that can result from exogenous insulin therapy.

Exogeneous insulin is beneficial for T1D management, but does not cure disease and requires daily blood glucose monitoring. Especially among paediatric patients, the burden of glucose management often leads to family-related stress and dramatically impacts a patient's quality of life. Patients optimized on insulin therapy still require extensive support to monitor daily food intake, to account for physical activity levels, to match carbohydrates to insulin needs, and to monitor glucose levels via multiple daily assessments. Maintaining blood glucose control while preserving a patient's quality of life thus remains a major challenge, especially among the paediatric population.

In newly diagnosed T1D, subjects may experience a brief period of remission, often beginning shortly after exogenous insulin therapy. Such remission is not certain, occurring only in ˜50% of children and adolescents, and is transient, with decline in glucose control resuming after several weeks to months. Without wishing to be bound by theory, intervention before the end of this remission period is expected to mitigate ongoing autoimmune responses to the pancreas while a substantial portion of functional islet cells remain, thereby reducing the need for exogenous insulin therapy. One such intervention, contemplated herein, are engineered T regulatory cells (EngTregs) specific to an islet cell antigen, for suppression of autoimmune responses with deleterious effects on islet cell function.

EngTregs as described herein comprise a modified TRAC locus in which an inserted heterologous promoter controls transcription of a first transmembrane protein component of a chemically induced signaling complex (CISC) containing an FK506-binding protein 12 (FKBP) extracellular domain and intracellular domain of IL-2Rγ, and a modified FOXP3 locus in which an inserted heterologous promoter controls transcription of a second transmembrane protein CISC component containing an FKBP-rapamycin-binding (FRB) domain and an intracellular domain of IL-2Rβ, such that IL-2 signal transduction occurs in the cell when exposed to rapamycin, resulting in proliferation of the cell in the presence of rapamycin. In some embodiments, the inserted heterologous promoter controls transcription of both the endogenous FOXP3 gene and the second transmembrane protein CISC component. Such chemically inducible proliferation of dual-edited cells allows efficient selection for and in vitro expansion of cells containing both modified loci, and thus both modifications associated with insertion of each CISC component. Specifically, the modified TRAC locus encodes, under transcriptional control of the inserted promoter, a heterologous TCRβ chain and a TCRα chain having a heterologous variable domain, such edited cells express a TCR specific to a peptide of the T1D-associated antigen IGRP. Moreover, the modified FOXP3 locus also encodes, under transcriptional control of the inserted promoter, a cytosolic FRB domain that binds intracellular rapamycin, preventing undesired effects (e.g., mTOR inhibition) of exposing cells to rapamycin for CISC-mediated IL-2 signal transduction. Finally, the heterologous promoter of the modified FOXP3 locus is inserted downstream from the Treg-specific demethylated region (TSDR) of the FOXP3 locus, and this inserted promoter controls transcription of an endogenous FOXP3 coding sequence independently of TSDR methylation that can occur in inflammatory environments. Bypassing TSDR-mediated silencing of FOXP3 expression by downstream promoter insertion allows a cell to maintain stable expression of FOXP3 even in inflammatory environments, which may otherwise inhibit FOXP3 expression and cause Treg cells to transdifferentiate into inflammatory T effector cells. Thus, the dual-edited cells described herein are T1D-associated antigen-specific Tregs, which both retain a stable suppressive phenotype in inflammatory environments (e.g., an inflamed pancreas), and may be expanded in a controllable manner in the presence of rapamycin.

Accordingly, some aspects of the disclosure relate to a method of producing a genetically modified cell, the method comprising contacting the cell with: (i) a first nucleic acid comprising: (a) a first 5′ homology arm having homology to a first nucleic acid sequence in a TRAC locus in the cell genome; (b) a first promoter, wherein the first promoter is an MND promoter; (c) a nucleotide sequence encoding a first chemically induced signaling complex (CISC) component comprising: (1) an extracellular binding domain comprising a rapamycin-binding domain of FK506-binding protein 12 (FKBP), (2) an IL-2Rγ transmembrane domain, and (3) an intracellular domain comprising an IL-2Rγ cytoplasmic domain a functional fragment thereof; (d) a nucleotide sequence encoding a TCRβ polypeptide or a functional fragment thereof; (e) a nucleotide sequence encoding at least a portion of a TCRα polypeptide, wherein the portion comprises a TCRα variable region and TCRα joining region, wherein a T cell receptor (TCR) comprising the TCRα and TCRβ polypeptides binds to a type 1 diabetes (T1D)-associated antigen; and (f) a first 3′ homology arm having homology to a second nucleic acid sequence in the TRAC locus that is downstream from the first nucleic acid sequence in the TRAC locus; and (ii) a second nucleic acid comprising: (a) a second 5′ homology arm having homology to a first nucleic acid sequence in a FOXP3 locus in the cell genome; (b) a second promoter, wherein the second promoter is an MND promoter; (c) a nucleotide sequence encoding a second CISC component comprising: (1) an extracellular binding domain comprising an FKBP-rapamycin-binding (FRB) domain of mTOR; (2) an IL-2Rβ transmembrane domain, and (3) an IL-2Rβ cytoplasmic domain or a functional fragment thereof; (d) a nucleotide sequence encoding a cytosolic FRB domain that binds rapamycin and does not comprise a transmembrane domain; and (e) a second 3′ homology arm having homology to a second nucleic acid sequence in the FOXP3 locus that is downstream from the first nucleic acid sequence in the FOXP3 locus, and downstream from a Treg-specific demethylated region (TSDR) in the FOXP3 locus.

In some embodiments, the first nucleic acid further comprises: a nucleotide sequence encoding a first 2A motif that is in-frame with and between the nucleotide sequences encoding the first CISC component and the TCRβ polypeptide; and a nucleotide sequence encoding a second 2A motif that is in-frame with between the nucleotide sequences encoding the TCRβ polypeptide and the at least portion of the TCRα polypeptide.

In some embodiments, the nucleotide sequence encoding the first 2A motif comprises no more than 90%, no more than 80%, no more than 70%, no more than 60%, or no more than 55% sequence identity to the nucleotide sequence encoding the second 2A motif.

In some embodiments, the first 2A motif is a T2A motif comprising the amino acid sequence of SEQ ID NO: 222, and the second 2A motif is a P2A motif comprising the amino acid sequence of SEQ ID NO: 226.

In some embodiments, the nucleotide sequence encoding the first 2A motif comprises at least 70% sequence identity to the nucleotide sequence of SEQ ID NO: 221, and the nucleotide sequence encoding the second 2A motif comprises at least 70% sequence identity to the nucleotide sequence of SEQ ID NO: 223.

In some embodiments, the second nucleic acid further comprises: a nucleotide sequence encoding a third 2A motif that is in-frame with between the nucleotide sequences encoding the second CISC component and the cytosolic FRB domain polypeptide; and a nucleotide sequence encoding a fourth 2A motif that is in-frame with between the nucleotide sequences encoding the cytosolic FRB domain polypeptide and the FoxP3 or portion thereof.

In some embodiments, the third 2A motif is a P2A motif comprising the amino acid sequence of SEQ ID NO: 227, and the fourth 2A motif is a P2A motif comprising the amino acid sequence of SEQ ID NO: 228.

In some embodiments, the nucleotide sequence encoding the third 2A motif comprises at least 70% sequence identity to the nucleotide sequence of SEQ ID NO: 224, and the nucleotide sequence encoding the fourth 2A motif comprises at least 70% sequence identity to the nucleotide sequence of SEQ ID NO: 225.

In some embodiments, the first CISC component further comprises a portion of an extracellular domain of IL-2Rγ.

In some embodiments, the second CISC component further comprises a portion of an extracellular domain of IL-2Rβ.

In some embodiments, the second CISC component comprises a threonine at a position corresponding to amino acid 2098 of wild-type mTOR having the amino acid sequence of SEQ ID NO: 236.

In some embodiments, the first CISC component comprises an amino acid sequence with at least 90%, at least 95%, at least 97%, at least 98%, at least 99%, or up to 100% sequence identity to the amino acid sequence of SEQ ID NO: 66.

In some embodiments, the second CISC component comprises an amino acid sequence with at least 90%, at least 95%, at least 97%, at least 98%, at least 99%, or up to 100% sequence identity to the amino acid sequence of SEQ ID NO: 71.

In some embodiments, the first CISC component comprises the amino acid sequence of SEQ ID NO: 66, and the second CISC component comprises the amino acid sequence of SEQ ID NO: 71.

In some embodiments, the nucleotide sequence encoding the at least portion of the TCRα polypeptide is inserted in-frame with an endogenous nucleotide sequence encoding at least a portion of a constant domain of the TCRα polypeptide, wherein the first MND promoter initiates transcription of a nucleotide sequence encoding the TCRα polypeptide comprising the TCRα variable region, TCRα joining region, and TCRα constant domain.

In some embodiments, the TCRβ polypeptide comprises: (i) (a) a CDR1 comprising the amino acid sequence of SEQ ID NO: 4; (b) a CDR2 comprising the amino acid sequence of SEQ ID NO: 5; and (c) a CDR3 comprising the amino acid sequence of SEQ ID NO: 6; (ii) (a) a CDR1 comprising the amino acid sequence of SEQ ID NO: 14; (b) a CDR2 comprising the amino acid sequence of SEQ ID NO: 15; and (c) a CDR3 comprising the amino acid sequence of SEQ ID NO: 16; or (iii) (a) a CDR1 comprising the amino acid sequence of SEQ ID NO: 24; (b) a CDR2 comprising the amino acid sequence of SEQ ID NO: 25; and (c) a CDR3 comprising the amino acid sequence of SEQ ID NO: 26.

In some embodiments, the TCRα polypeptide comprises: (i) (a) a CDR1 comprising the amino acid sequence of SEQ ID NO: 1; (b) a CDR2 comprising the amino acid sequence of SEQ ID NO: 2; and (c) a CDR3 comprising the amino acid sequence of SEQ ID NO: 3; (ii) (a) a CDR1 comprising the amino acid sequence of SEQ ID NO: 11; (b) a CDR2 comprising the amino acid sequence of SEQ ID NO: 12; and (c) a CDR3 comprising the amino acid sequence of SEQ ID NO: 13; or (iii) (a) a CDR1 comprising the amino acid sequence of SEQ ID NO: 21; (b) a CDR2 comprising the amino acid sequence of SEQ ID NO: 22; and (c) a CDR3 comprising the amino acid sequence of SEQ ID NO: 23.

In some embodiments, the TCRα polypeptide comprises a variable domain comprising the amino acid sequence of any one of SEQ ID NOs: 7, 17, and 27.

In some embodiments, the TCRβ polypeptide comprises a variable domain comprising the amino acid sequence of any one of SEQ ID NOs: 8, 18, and 28.

In some embodiments: (i) the TCRα polypeptide comprises an αCDR1 having the amino acid sequence of SEQ ID NO: 1, an αCDR2 having the amino acid sequence of SEQ ID NO: 2, and an αCDR3 having the amino acid sequence of SEQ ID NO: 3; and the TCRβ polypeptide comprises a bCDR1 having the amino acid sequence of SEQ ID NO: 4, a bCDR2 having the amino acid sequence of SEQ ID NO: 5, and a bCDR3 having an amino acid sequence of SEQ ID NO: 6; (ii) the TCRα polypeptide comprises an αCDR1 having the amino acid sequence of SEQ ID NO: 11, an αCDR2 having the amino acid sequence of SEQ ID NO: 12, and an αCDR3 having the amino acid sequence of SEQ ID NO: 13; and the TCRβ polypeptide comprises a bCDR1 having the amino acid sequence of SEQ ID NO: 14, a bCDR2 having the amino acid sequence of SEQ ID NO: 15, and a bCDR3 having an amino acid sequence of SEQ ID NO: 16; or (iii) the TCRα polypeptide comprises an αCDR1 having the amino acid sequence of SEQ ID NO: 21, an αCDR2 having the amino acid sequence of SEQ ID NO: 22, and an αCDR3 having the amino acid sequence of SEQ ID NO: 23; and the TCRβ polypeptide comprises a bCDR1 having the amino acid sequence of SEQ ID NO: 24, a bCDR2 having the amino acid sequence of SEQ ID NO: 25, and a bCDR3 having an amino acid sequence of SEQ ID NO: 26.

In some embodiments: (i) the TCRα polypeptide comprises a variable domain comprising the amino acid sequence of SEQ ID NO: 7, and the TCRβ polypeptide comprises a variable domain comprising the amino acid sequence of SEQ ID NO: 8; (ii) the TCRα polypeptide comprises a variable domain comprising the amino acid sequence of SEQ ID NO: 17, and the TCRβ polypeptide comprises a variable domain comprising the amino acid sequence of SEQ ID NO: 18; or (iii) the TCRα polypeptide comprises a variable domain comprising the amino acid sequence of SEQ ID NO: 27, and the TCRβ polypeptide comprises a variable domain comprising the amino acid sequence of SEQ ID NO: 28. In some embodiments: (i) the TCRα polypeptide comprises the amino acid sequence of SEQ ID NO: 9, and the TCRβ polypeptide comprises the amino acid sequence of SEQ ID NO: 10; (ii) the TCRα polypeptide comprises the amino acid sequence of SEQ ID NO: 19, and the TCRβ polypeptide comprises the amino acid sequence of SEQ ID NO: 20; or (iii) the TCRα polypeptide comprises the amino acid sequence of SEQ ID NO: 29, and the TCRβ polypeptide comprises the amino acid sequence of SEQ ID NO: 30.

In some embodiments, insertion of the second nucleic acid into the cell genome modifies the sequence of a first coding exon in the FOXP3 locus.

In some embodiments, insertion of the second nucleic acid into the cell genome does not change the nucleotide sequence of a first coding exon of the FOXP3 locus.

In some embodiments, the method further comprises contacting the cell with a DNA endonuclease or a third nucleic acid encoding the DNA endonuclease.

In some embodiments, the third nucleic acid encoding the DNA endonuclease is an RNA.

In some embodiments, the RNA encoding the DNA endonuclease is an mRNA.

In some embodiments, the DNA endonuclease is an RNA-guided DNA endonuclease.

In some embodiments, the RNA-guided DNA endonuclease is a Cas endonuclease.

In some embodiments, the Cas endonuclease is a Cas9 endonuclease.

In some embodiments, the method comprises contacting the cell with a TRAC locus-targeting guide RNA (gRNA) comprising a spacer sequence that is complementary to a sequence within the TRAC locus, or a fourth nucleic acid encoding the TRAC locus-targeting gRNA.

In some embodiments, the 5′ homology arm of the first nucleic acid comprises a sequence with at least 90% sequence identity to SEQ ID NO: 85, and the 3′ homology arm of the first nucleic acid comprises a sequence with at least 90% sequence identity to SEQ ID NO: 93.

In some embodiments, the 5′ homology arm of the first nucleic acid comprises a sequence with at least 90% sequence identity to SEQ ID NO: 96, and the 3′ homology arm of the first nucleic acid comprises a sequence with at least 90% sequence identity to SEQ ID NO: 105.

In some embodiments, the 5′ homology arm of the first nucleic acid comprises a sequence with at least 90% sequence identity to SEQ ID NO: 108, and the 3′ homology arm of the first nucleic acid comprises a sequence with at least 90% sequence identity to SEQ ID NO: 116.

In some embodiments, the 5′ homology arm of the first nucleic acid comprises a sequence with at least 90% sequence identity to SEQ ID NO: 119, and the 3′ homology arm of the first nucleic acid comprises a sequence with at least 90% sequence identity to SEQ ID NO: 127.

In some embodiments, the 5′ homology arm of the first nucleic acid comprises a sequence with at least 90% sequence identity to SEQ ID NO: 130, and the 3′ homology arm of the first nucleic acid comprises a sequence with at least 90% sequence identity to SEQ ID NO: 138.

In some embodiments, the method further comprises contacting the cell with a FOXP3 locus-targeting guide RNA (gRNA) comprising a spacer sequence that is complementary to a sequence within the FOXP3 locus, or a fourth nucleic acid encoding the FOXP3 locus-targeting gRNA.

In some embodiments, the 5′ homology arm of the second nucleic acid comprises a sequence with at least 90% sequence identity to SEQ ID NO: 141, and the 3′ homology arm of the second nucleic acid comprises a sequence with at least 90% sequence identity to SEQ ID NO: 149.

In some embodiments, the 5′ homology arm of the second nucleic acid comprises a sequence with at least 90% sequence identity to SEQ ID NO: 152, and the 3′ homology arm of the second nucleic acid comprises a sequence with at least 90% sequence identity to SEQ ID NO: 160.

In some embodiments, the 5′ homology arm of the second nucleic acid comprises a sequence with at least 90% sequence identity to SEQ ID NO: 163, and the 3′ homology arm of the second nucleic acid comprises a sequence with at least 90% sequence identity to SEQ ID NO: 171.

In some embodiments, the 5′ homology arm of the second nucleic acid comprises a sequence with at least 90% sequence identity to SEQ ID NO: 174, and the 3′ homology arm of the second nucleic acid comprises a sequence with at least 90% sequence identity to SEQ ID NO: 183.

In some embodiments, the 5′ homology arm of the second nucleic acid comprises a sequence with at least 90% sequence identity to SEQ ID NO: 186, and the 3′ homology arm of the second nucleic acid comprises a sequence with at least 90% sequence identity to SEQ ID NO: 194.

In some embodiments, the 5′ homology arm of the second nucleic acid comprises a sequence with at least 90% sequence identity to SEQ ID NO: 197, and the 3′ homology arm of the second nucleic acid comprises a sequence with at least 90% sequence identity to SEQ ID NO: 205.

In some embodiments, the 5′ homology arm of the second nucleic acid comprises a sequence with at least 90% sequence identity to SEQ ID NO: 208, and the 3′ homology arm of the second nucleic acid comprises a sequence with at least 90% sequence identity to SEQ ID NO: 217.

In some embodiments, the first nucleic acid is comprised within a first vector.

In some embodiments, the first vector is an adeno-associated virus (AAV) vector.

In some embodiments, the first vector is an AAV vector derived from an AAV of serotype AAV1, AAV2, AAV3, AAV4, AAV5, AAV6, AAV7, AAV8, AAV9, AAV10, or AAV11.

In some embodiments, the second nucleic acid is comprised within a second vector.

In some embodiments, the second vector is an adeno-associated virus (AAV) vector.

In some embodiments, the second vector is an AAV vector derived from an AAV of serotype AAV1, AAV2, AAV3, AAV4, AAV5, AAV6, AAV7, AAV8, AAV9, AAV10, or AAV11.

In some embodiments, the first nucleic acid comprises, between the first 5′ and 3′ homology arms, a nucleotide sequence having at least 95% sequence identity to any one of SEQ ID NOs: 94, 106, 117, 128, and 139.

In some embodiments, the second nucleic acid comprises, between the first 5′ and 3′ homology arms, a nucleotide sequence having at least 95% sequence identity to any one of SEQ ID NOs: 150, 161, 172, 184, 195, 206, and 218.

In some embodiments, the first nucleic acid comprises a nucleotide sequence having at least 95% sequence identity to any one of SEQ ID NOs: 95, 107, 118, 129, and 140.

In some embodiments, the second nucleic acid comprises a nucleotide sequence having at least 95% sequence identity to any one of SEQ ID NOs: 151, 162, 173, 185, 196, 207, and 219.

In some embodiments, one or more of the homology arms is 100-2000 nucleotides in length.

In some embodiments, each of the homology arms is 300-700 nucleotides in length.

Some aspects of the disclosure relate to a genetically modified cell made by a method describe herein.

Some aspects of the disclosure relate to a genetically modified cell comprising: (i) a first inserted nucleic acid in a TRAC locus of the cell genome, wherein the TRAC locus comprises: (a) a first promoter, wherein the first promoter is an MND promoter; (b) an exogenous nucleotide sequence encoding a first chemically induced signaling complex (CISC) component comprising: (1) an extracellular binding domain comprising a rapamycin-binding domain of FK506-binding protein 12 (FKBP), (2) an IL-2Rγ transmembrane domain, and (3) an intracellular domain comprising an IL-2Rγ cytoplasmic domain a functional fragment thereof; (c) an exogenous nucleotide sequence encoding an exogenous TCRβ polypeptide or a functional fragment thereof; (d) an exogenous nucleotide sequence encoding at least a portion of a TCRα polypeptide, wherein the portion comprises a TCRα variable region and TCRα joining region, wherein a T cell receptor (TCR) comprising the TCRα and TCRβ polypeptides binds to a type 1 diabetes (T1D)-associated antigen; and (ii) a second inserted nucleic acid in a FOXP3 locus of the cell genome, wherein the FOXP3 locus comprises: (a) a second promoter, wherein the second promoter is an MND promoter; (b) a nucleotide sequence encoding a second CISC component comprising: (1) an extracellular binding domain comprising an FKBP-rapamycin-binding (FRB) domain of mTOR; (2) an IL-2Rβ transmembrane domain, and (3) an IL-2Rβ cytoplasmic domain or a functional fragment thereof; (c) a nucleotide sequence encoding a cytosolic FRB domain that binds rapamycin and does not comprise a transmembrane domain, wherein the second MND promoter is inserted downstream from a Treg-specific demethylated region of the FOXP3 locus, and initiates transcription of an endogenous nucleotide sequence encoding FoxP3 or a portion thereof.

In some embodiments, the first nucleic acid further comprises: a nucleotide sequence encoding a first 2A motif that is in-frame with and between the nucleotide sequences encoding the first CISC component and the TCRβ polypeptide; and a nucleotide sequence encoding a second 2A motif that is in-frame with between the nucleotide sequences encoding the TCRβ polypeptide and the at least portion of the TCRα polypeptide.

In some embodiments, the nucleotide sequence encoding the first 2A motif comprises no more than 90%, no more than 80%, no more than 70%, no more than 60%, or no more than 55% sequence identity to the nucleotide sequence encoding the second 2A motif.

In some embodiments, the first 2A motif is a T2A motif comprising the amino acid sequence of SEQ ID NO: 222, and the second 2A motif is a P2A motif comprising the amino acid sequence of SEQ ID NO: 226.

In some embodiments, the nucleotide sequence encoding the first 2A motif comprises at least 70% sequence identity to the nucleotide sequence of SEQ ID NO: 221, and the nucleotide sequence encoding the second 2A motif comprises at least 70% sequence identity to the nucleotide sequence of SEQ ID NO: 223.

In some embodiments, the second nucleic acid further comprises: a nucleotide sequence encoding a third 2A motif that is in-frame with between the nucleotide sequences encoding the second CISC component and the cytosolic FRB domain polypeptide; and a nucleotide sequence encoding a fourth 2A motif that is in-frame with between the nucleotide sequences encoding the cytosolic FRB domain polypeptide and the FoxP3 or portion thereof.

In some embodiments, the third 2A motif is a P2A motif comprising the amino acid sequence of SEQ ID NO: 227, and the fourth 2A motif is a P2A motif comprising the amino acid sequence of SEQ ID NO: 228.

In some embodiments, the nucleotide sequence encoding the third 2A motif comprises at least 70% sequence identity to the nucleotide sequence of SEQ ID NO: 224, and the nucleotide sequence encoding the fourth 2A motif comprises at least 70% sequence identity to the nucleotide sequence of SEQ ID NO: 225.

In some embodiments, the first CISC component further comprises a portion of an extracellular domain of IL-2Rγ.

In some embodiments, the second CISC component further comprises a portion of an extracellular domain of IL-2Rβ.

In some embodiments, the second CISC component comprises a threonine at a position corresponding to amino acid 2098 of wild-type mTOR having the amino acid sequence of SEQ ID NO: 236.

In some embodiments, the first CISC component comprises an amino acid sequence with at least 90%, at least 95%, at least 97%, at least 98%, at least 99%, or up to 100% sequence identity to the amino acid sequence of SEQ ID NO: 66.

In some embodiments, the second CISC component comprises an amino acid sequence with at least 90%, at least 95%, at least 97%, at least 98%, at least 99%, or up to 100% sequence identity to the amino acid sequence of SEQ ID NO: 71.

In some embodiments, the first CISC component comprises the amino acid sequence of SEQ ID NO: 66, and the second CISC component comprises the amino acid sequence of SEQ ID NO: 71.

In some embodiments, the nucleotide sequence encoding the at least portion of the TCRα polypeptide is inserted in-frame with an endogenous nucleotide sequence encoding at least a portion of a constant domain of the TCRα polypeptide, wherein the first MND promoter initiates transcription of a nucleotide sequence encoding the TCRα polypeptide comprising the TCRα variable region, TCRα joining region, and TCRα constant domain.

In some embodiments, the TCRβ polypeptide comprises: (i) (a) a CDR1 comprising the amino acid sequence of SEQ ID NO: 4; (b) a CDR2 comprising the amino acid sequence of SEQ ID NO: 5; and (c) a CDR3 comprising the amino acid sequence of SEQ ID NO: 6; (ii) (a) a CDR1 comprising the amino acid sequence of SEQ ID NO: 14; (b) a CDR2 comprising the amino acid sequence of SEQ ID NO: 15; and (c) a CDR3 comprising the amino acid sequence of SEQ ID NO: 16; or (iii) (a) a CDR1 comprising the amino acid sequence of SEQ ID NO: 24; (b) a CDR2 comprising the amino acid sequence of SEQ ID NO: 25; and (c) a CDR3 comprising the amino acid sequence of SEQ ID NO: 26.

In some embodiments, the TCRα polypeptide comprises: (i) (a) a CDR1 comprising the amino acid sequence of SEQ ID NO: 1; (b) a CDR2 comprising the amino acid sequence of SEQ ID NO: 2; and (c) a CDR3 comprising the amino acid sequence of SEQ ID NO: 3; (ii) (a) a CDR1 comprising the amino acid sequence of SEQ ID NO: 11; (b) a CDR2 comprising the amino acid sequence of SEQ ID NO: 12; and (c) a CDR3 comprising the amino acid sequence of SEQ ID NO: 13; or (iii) (a) a CDR1 comprising the amino acid sequence of SEQ ID NO: 21; (b) a CDR2 comprising the amino acid sequence of SEQ ID NO: 22; and (c) a CDR3 comprising the amino acid sequence of SEQ ID NO: 23.

In some embodiments, the TCRα polypeptide comprises a variable domain comprising the amino acid sequence of any one of SEQ ID NOs: 7, 17, and 27.

In some embodiments, the TCRβ polypeptide comprises a variable domain comprising the amino acid sequence of any one of SEQ ID NOs: 8, 18, and 28.

In some embodiments: (i) the TCRα polypeptide comprises an αCDR1 having the amino acid sequence of SEQ ID NO: 1, an αCDR2 having the amino acid sequence of SEQ ID NO: 2, and an αCDR3 having the amino acid sequence of SEQ ID NO: 3; and the TCRβ polypeptide comprises a bCDR1 having the amino acid sequence of SEQ ID NO: 4, a bCDR2 having the amino acid sequence of SEQ ID NO: 5, and a bCDR3 having an amino acid sequence of SEQ ID NO: 6; (ii) the TCRα polypeptide comprises an αCDR1 having the amino acid sequence of SEQ ID NO: 11, an αCDR2 having the amino acid sequence of SEQ ID NO: 12, and an αCDR3 having the amino acid sequence of SEQ ID NO: 13; and the TCRβ polypeptide comprises a bCDR1 having the amino acid sequence of SEQ ID NO: 14, a bCDR2 having the amino acid sequence of SEQ ID NO: 15, and a bCDR3 having an amino acid sequence of SEQ ID NO: 16; or (iii) the TCRα polypeptide comprises an αCDR1 having the amino acid sequence of SEQ ID NO: 21, an αCDR2 having the amino acid sequence of SEQ ID NO: 22, and an αCDR3 having the amino acid sequence of SEQ ID NO: 23; and the TCRβ polypeptide comprises a bCDR1 having the amino acid sequence of SEQ ID NO: 24, a bCDR2 having the amino acid sequence of SEQ ID NO: 25, and a bCDR3 having an amino acid sequence of SEQ ID NO: 26.

In some embodiments: (i) the TCRα polypeptide comprises a variable domain comprising the amino acid sequence of SEQ ID NO: 7, and the TCRβ polypeptide comprises a variable domain comprising the amino acid sequence of SEQ ID NO: 8; (ii) the TCRα polypeptide comprises a variable domain comprising the amino acid sequence of SEQ ID NO: 17, and the TCRβ polypeptide comprises a variable domain comprising the amino acid sequence of SEQ ID NO: 18; or (iii) the TCRα polypeptide comprises a variable domain comprising the amino acid sequence of SEQ ID NO: 27, and the TCRβ polypeptide comprises a variable domain comprising the amino acid sequence of SEQ ID NO: 28.

In some embodiments: (i) the TCRα polypeptide comprises the amino acid sequence of SEQ ID NO: 9, and the TCRβ polypeptide comprises the amino acid sequence of SEQ ID NO: 10; (ii) the TCRα polypeptide comprises the amino acid sequence of SEQ ID NO: 19, and the TCRβ polypeptide comprises the amino acid sequence of SEQ ID NO: 20; or (iii) the TCRα polypeptide comprises the amino acid sequence of SEQ ID NO: 29, and the TCRβ polypeptide comprises the amino acid sequence of SEQ ID NO: 30.

In some embodiments, insertion of the second nucleic acid into the cell genome modifies the sequence of a first coding exon in the FOXP3 locus.

In some embodiments, insertion of the second nucleic acid into the cell genome does not change the nucleotide sequence of a first coding exon of the FOXP3 locus.

In some embodiments, the genetically modified cell is a CD3+, CD4+, and/or CD8+ T cell.

In some embodiments, the genetically modified cell is a CD4+ T cell.

In some embodiments, the genetically modified cell is a Treg cell.

In some embodiments, the genetically modified cell is a FoxP3+ Treg cell.

In some embodiments, the genetically modified cell is CTLA-4+, LAG-3+, CD25+, CD39+, CD27+, CD70+, GITR+, neuropilin-1+, galectin-1+, and/or IL-2Rα+.

Some aspects of the disclosure relate to a pharmaceutical composition comprising a genetically modified cell described herein, and a pharmaceutically acceptable excipient.

Some aspects of the disclosure relate to a method comprising administering a pharmaceutical composition or genetically modified cell described herein to a subject.

In some embodiments, the genetically modified cell is autologous to the subject.

In some embodiments, the genetically modified cell is allogeneic to the subject.

In some embodiments, the subject has type 1 diabetes (T1D).

In some embodiments, the subject has been diagnosed with T1D no more than 6 months, no more than 5 months, no more than 4 months, no more than 3 months, no more than 3 months, no more than 2 months, or no more than 1 month before administration of the cell.

In some embodiments, the subject has an insulin dose-adjusted hemoglobin A1c (IDAA1c) of 9.0 or lower.

In some embodiments, after the subject has been diagnosed with T1D, the IDAA1c of the subject has decreased from above 9.0 to 9.0 or lower.

In some embodiments, autoantibodies that bind an antigen selected from the group consisting of islet cell antigen, insulin, glutamic acid decarboxylase, islet tyrosine phosphatase 2, and/or zinc transporter 8 have been detected in the subject no more than 6 months, no more than 5 months, no more than 4 months, no more than 3 months, no more than 3 months, no more than 2 months, or no more than 1 month before administration of the cell.

In some embodiments, the subject has not been diagnosed with type 1 diabetes (T1D).

In some embodiments, the subject has a hemoglobin A1c of 5.7 to 6.4.

In some embodiments, the subject has a hemoglobin A1c of 6.5 or higher.

In some embodiments, the subject is at least 3 years, but less than 6 years, old, and is administered a dose comprising 1×10⁸ to 6×10⁸ of the cells.

In some embodiments, the dose comprises 2.4×10⁸ to 3.6×10⁸ of the cells.

In some embodiments, the dose comprises about 3×10⁸ of the cells.

In some embodiments, the subject is at least 6 years, but less than 12 years, old, and is administered a dose comprising 2×10⁸ to 1×10⁹ of the cells.

In some embodiments, the dose comprises 4×10⁸ to 6×10⁸ of the cells.

In some embodiments, the dose comprises about 5×10⁸ of the cells.

In some embodiments, the subject is at least 12 years, but less than 18 years, old, and is administered a dose comprising 5×10⁸ to 2×10⁹ of the cells.

In some embodiments, the dose comprises 8×10⁸ to 1.2×10⁹ of the cells.

In some embodiments, the dose comprises about 10⁹ of the cells.

In some embodiments, the subject is at least 18 years old, and is administered a dose comprising 5×10⁸ to 2×10⁹ of the cells.

In some embodiments, the subject is less than 46 years old.

In some embodiments, the dose comprises 8×10⁸ to 1.2×10⁹ of the cells.

In some embodiments, the dose comprises about 10⁹ of the cells.

In some embodiments, the subject has an estimated pancreatic volume determined by age of the subject, wherein the subject is administered a dose of: (a) 1×10⁸ to 6×10⁸ of the cells if the estimated pancreatic volume is about 20 mL; (b) 2×10⁸ to 1×10⁹ of the cells if the estimated pancreatic volume is about 35 mL; or (c) 5×10⁸ to 2×10⁹ of the cells if the estimated pancreatic volume is about 60 mL or higher.

In some embodiments, the subject is administered a dose of: (a) 2.4×10⁸ to 3.6×10⁸ of the cells if the estimated pancreatic volume is about 20 mL; (b) 4×10⁸ to 6×10⁸ of the cells if the estimated pancreatic volume is about 35 mL; or (c) 8×10⁸ to 1.2×10⁹ of the cells if the estimated pancreatic volume is about 60 mL or higher.

In some embodiments, the subject is administered a dose of: (a) about 3×10⁸ of the cells if the estimated pancreatic volume is about 20 mL; (b) about 5×10⁸ of the cells if the estimated pancreatic volume is about 35 mL; or (c) about 10⁹ of the cells if the estimated pancreatic volume is 60 mL or higher.

In some embodiments, the subject has an estimated pancreatic volume determined by age of the subject, wherein the method further comprises measuring an actual pancreatic volume of the subject, wherein the subject is administered a dose of the cells that is between: (a) (a ratio of the actual:estimated pancreatic volumes of the subject)*(1×10⁸ to 6×10⁸) if the estimated pancreatic volume is about 20 mL; (b) (the ratio of the actual:estimated pancreatic volumes of the subject)*(2×10⁸ to 1×10⁹) if the estimated pancreatic volume is about 35 mL; or (c) (the ratio of the actual:estimated pancreatic volumes of the subject)*(5×10⁸ to 2×10⁹) if the estimated pancreatic volume is about 60 mL or higher.

In some embodiments, the subject is administered a dose of the cells that is between: (a) (the ratio of the actual:estimated pancreatic volumes of the subject)*(2.4×10⁸ to 3.6×10⁸) if the estimated pancreatic volume is about 20 mL; (b) (the ratio of the actual:estimated pancreatic volumes of the subject)*(4×10⁸ to 6×10⁸) if the estimated pancreatic volume is about 35 mL; or (c) (the ratio of the actual:estimated pancreatic volumes of the subject)*(8×10⁸ to 1.2×10⁹) if the estimated pancreatic volume is about 60 mL or higher.

In some embodiments, the subject is administered a dose of the cells that is between: (a) about (the ratio of the actual:estimated pancreatic volumes of the subject)*(3×10⁸) if the estimated pancreatic volume is about 20 mL; (b) about (the ratio of the actual:estimated pancreatic volumes of the subject)*(5×10⁸) if the estimated pancreatic volume is about 35 mL; or (c) about (the ratio of the actual:estimated pancreatic volumes of the subject)*(10⁹) if the estimated pancreatic volume is about 60 mL or higher.

In some embodiments, the subject is a human.

Some aspects of the disclosure relate to a system comprising: (i) a first nucleic acid comprising: (a) a first 5′ homology arm having homology to a first nucleic acid sequence in a TRAC locus in the cell genome; (b) a first promoter, wherein the first promoter is an MND promoter; (c) a nucleotide sequence encoding a first chemically induced signaling complex (CISC) component comprising: (1) an extracellular binding domain comprising a rapamycin-binding domain of FK506-binding protein 12 (FKBP), (2) an IL-2Rγ transmembrane domain, and (3) an intracellular domain comprising an IL-2Rγ cytoplasmic domain a functional fragment thereof; (d) a nucleotide sequence encoding a TCRβ polypeptide or a functional fragment thereof; (e) a nucleotide sequence encoding at least a portion of a TCRα polypeptide, wherein the portion comprises a TCRα variable region and TCRα joining region, wherein a T cell receptor (TCR) comprising the TCRα and TCRβ polypeptides binds to a type 1 diabetes (T1D)-associated antigen; and (f) a first 3′ homology arm having homology to a second nucleic acid sequence in the TRAC locus that is downstream from the first nucleic acid sequence in the TRAC locus; (ii) a second nucleic acid comprising: (a) a second 5′ homology arm having homology to a first nucleic acid sequence in a FOXP3 locus in the cell genome; (b) a second promoter, wherein the second promoter is an MND promoter; (c) a nucleotide sequence encoding a second CISC component comprising: (1) an extracellular binding domain comprising an FKBP-rapamycin-binding (FRB) domain of mTOR; (2) an IL-2Rβ transmembrane domain, and (3) an IL-2Rβ cytoplasmic domain or a functional fragment thereof; (d) a nucleotide sequence encoding a cytosolic FRB domain that binds rapamycin and does not comprise a transmembrane domain; and (e) a second 3′ homology arm having homology to a second nucleic acid sequence in the FOXP3 locus that is downstream from the first nucleic acid sequence in the FOXP3 locus, and downstream from a Treg-specific demethylated region (TSDR) in the FOXP3 locus.

In some embodiments, the first nucleic acid further comprises: a nucleotide sequence encoding a first 2A motif that is in-frame with and between the nucleotide sequences encoding the first CISC component and the TCRβ polypeptide; and a nucleotide sequence encoding a second 2A motif that is in-frame with between the nucleotide sequences encoding the TCRβ polypeptide and the at least portion of the TCRα polypeptide.

In some embodiments, the nucleotide sequence encoding the first 2A motif comprises no more than 90%, no more than 80%, no more than 70%, no more than 60%, or no more than 55% sequence identity to the nucleotide sequence encoding the second 2A motif.

In some embodiments, the first 2A motif is a T2A motif comprising the amino acid sequence of SEQ ID NO: 222, and the second 2A motif is a P2A motif comprising the amino acid sequence of SEQ ID NO: 226.

In some embodiments, the nucleotide sequence encoding the first 2A motif comprises at least 70% sequence identity to the nucleotide sequence of SEQ ID NO: 221, and the nucleotide sequence encoding the second 2A motif comprises at least 70% sequence identity to the nucleotide sequence of SEQ ID NO: 223.

In some embodiments, the second nucleic acid further comprises: a nucleotide sequence encoding a third 2A motif that is in-frame with between the nucleotide sequences encoding the second CISC component and the cytosolic FRB domain polypeptide; and a nucleotide sequence encoding a fourth 2A motif that is in-frame with between the nucleotide sequences encoding the cytosolic FRB domain polypeptide and the FoxP3 or portion thereof.

In some embodiments, the third 2A motif is a P2A motif comprising the amino acid sequence of SEQ ID NO: 227, and the fourth 2A motif is a P2A motif comprising the amino acid sequence of SEQ ID NO: 228.

In some embodiments, the nucleotide sequence encoding the third 2A motif comprises at least 70% sequence identity to the nucleotide sequence of SEQ ID NO: 224, and the nucleotide sequence encoding the fourth 2A motif comprises at least 70% sequence identity to the nucleotide sequence of SEQ ID NO: 225.

In some embodiments, the first CISC component further comprises a portion of an extracellular domain of IL-2Rγ.

In some embodiments, the second CISC component further comprises a portion of an extracellular domain of IL-2Rβ.

In some embodiments, the second CISC component comprises a threonine at a position corresponding to amino acid 2098 of wild-type mTOR having the amino acid sequence of SEQ ID NO: 236.

In some embodiments, the first CISC component comprises an amino acid sequence with at least 90%, at least 95%, at least 97%, at least 98%, at least 99%, or up to 100% sequence identity to the amino acid sequence of SEQ ID NO: 66.

In some embodiments, the second CISC component comprises an amino acid sequence with at least 90%, at least 95%, at least 97%, at least 98%, at least 99%, or up to 100% sequence identity to the amino acid sequence of SEQ ID NO: 71.

In some embodiments, the first CISC component comprises the amino acid sequence of SEQ ID NO: 66, and the second CISC component comprises the amino acid sequence of SEQ ID NO: 71.

In some embodiments, the nucleotide sequence encoding the at least portion of the TCRα polypeptide is in-frame with a nucleotide sequence in the 3′ homology arm encoding at least a portion of a constant domain of the TCRα polypeptide, wherein the first MND promoter initiates transcription of a nucleotide sequence encoding the TCRα polypeptide comprising the TCRα variable region, TCRα joining region, and TCRα constant domain.

In some embodiments, the TCRβ polypeptide comprises: (i) (a) a CDR1 comprising the amino acid sequence of SEQ ID NO: 4; (b) a CDR2 comprising the amino acid sequence of SEQ ID NO: 5; and (c) a CDR3 comprising the amino acid sequence of SEQ ID NO: 6; (ii) (a) a CDR1 comprising the amino acid sequence of SEQ ID NO: 14; (b) a CDR2 comprising the amino acid sequence of SEQ ID NO: 15; and (c) a CDR3 comprising the amino acid sequence of SEQ ID NO: 16; or (iii) (a) a CDR1 comprising the amino acid sequence of SEQ ID NO: 24; (b) a CDR2 comprising the amino acid sequence of SEQ ID NO: 25; and (c) a CDR3 comprising the amino acid sequence of SEQ ID NO: 26.

In some embodiments, the TCRα polypeptide comprises: (i) (a) a CDR1 comprising the amino acid sequence of SEQ ID NO: 1; (b) a CDR2 comprising the amino acid sequence of SEQ ID NO: 2; and (c) a CDR3 comprising the amino acid sequence of SEQ ID NO: 3; (ii) (a) a CDR1 comprising the amino acid sequence of SEQ ID NO: 11; (b) a CDR2 comprising the amino acid sequence of SEQ ID NO: 12; and (c) a CDR3 comprising the amino acid sequence of SEQ ID NO: 13; or (iii) (a) a CDR1 comprising the amino acid sequence of SEQ ID NO: 21; (b) a CDR2 comprising the amino acid sequence of SEQ ID NO: 22; and (c) a CDR3 comprising the amino acid sequence of SEQ ID NO: 23.

In some embodiments, the TCRα polypeptide comprises a variable domain comprising the amino acid sequence of any one of SEQ ID NOs: 7, 17, and 27.

In some embodiments, the TCRβ polypeptide comprises a variable domain comprising the amino acid sequence of any one of SEQ ID NOs: 8, 18, and 28.

In some embodiments: (i) the TCRα polypeptide comprises an αCDR1 having the amino acid sequence of SEQ ID NO: 1, an αCDR2 having the amino acid sequence of SEQ ID NO: 2, and an αCDR3 having the amino acid sequence of SEQ ID NO: 3; and the TCRβ polypeptide comprises a bCDR1 having the amino acid sequence of SEQ ID NO: 4, a bCDR2 having the amino acid sequence of SEQ ID NO: 5, and a bCDR3 having an amino acid sequence of SEQ ID NO: 6; (ii) the TCRα polypeptide comprises an αCDR1 having the amino acid sequence of SEQ ID NO: 11, an αCDR2 having the amino acid sequence of SEQ ID NO: 12, and an αCDR3 having the amino acid sequence of SEQ ID NO: 13; and the TCRβ polypeptide comprises a bCDR1 having the amino acid sequence of SEQ ID NO: 14, a bCDR2 having the amino acid sequence of SEQ ID NO: 15, and a bCDR3 having an amino acid sequence of SEQ ID NO: 16; or (iii) the TCRα polypeptide comprises an αCDR1 having the amino acid sequence of SEQ ID NO: 21, an αCDR2 having the amino acid sequence of SEQ ID NO: 22, and an αCDR3 having the amino acid sequence of SEQ ID NO: 23; and the TCRβ polypeptide comprises a bCDR1 having the amino acid sequence of SEQ ID NO: 24, a bCDR2 having the amino acid sequence of SEQ ID NO: 25, and a bCDR3 having an amino acid sequence of SEQ ID NO: 26.

In some embodiments: (i) the TCRα polypeptide comprises a variable domain comprising the amino acid sequence of SEQ ID NO: 7, and the TCRβ polypeptide comprises a variable domain comprising the amino acid sequence of SEQ ID NO: 8; (ii) the TCRα polypeptide comprises a variable domain comprising the amino acid sequence of SEQ ID NO: 17, and the TCRβ polypeptide comprises a variable domain comprising the amino acid sequence of SEQ ID NO: 18; or (iii) the TCRα polypeptide comprises a variable domain comprising the amino acid sequence of SEQ ID NO: 27, and the TCRβ polypeptide comprises a variable domain comprising the amino acid sequence of SEQ ID NO: 28.

In some embodiments: (i) the TCRα polypeptide comprises the amino acid sequence of SEQ ID NO: 9, and the TCRβ polypeptide comprises the amino acid sequence of SEQ ID NO: 10; (ii) the TCRα polypeptide comprises the amino acid sequence of SEQ ID NO: 19, and the TCRβ polypeptide comprises the amino acid sequence of SEQ ID NO: 20; or (iii) the TCRα polypeptide comprises the amino acid sequence of SEQ ID NO: 29, and the TCRβ polypeptide comprises the amino acid sequence of SEQ ID NO: 30.

In some embodiments, insertion of the second nucleic acid into a cell genome modifies the sequence of a first coding exon in the FOXP3 locus.

In some embodiments, insertion of the second nucleic acid into a cell genome does not change the nucleotide sequence of a first coding exon of the FOXP3 locus.

In some embodiments, the system further comprises a DNA endonuclease or a third nucleic acid encoding the DNA endonuclease.

In some embodiments, the third nucleic acid encoding the DNA endonuclease is an RNA.

In some embodiments, the RNA encoding the DNA endonuclease is an mRNA.

In some embodiments, the DNA endonuclease is an RNA-guided DNA endonuclease.

In some embodiments, the RNA-guided DNA endonuclease is a Cas endonuclease.

In some embodiments, the Cas endonuclease is a Cas9 endonuclease.

In some embodiments, the system further comprises a TRAC locus-targeting guide RNA (gRNA) comprising a spacer sequence that is complementary to a sequence within the TRAC locus, or a fourth nucleic acid encoding the TRAC locus-targeting gRNA.

In some embodiments, the 5′ homology arm of the first nucleic acid comprises a sequence with at least 90% sequence identity to SEQ ID NO: 85, and the 3′ homology arm of the first nucleic acid comprises a sequence with at least 90% sequence identity to SEQ ID NO: 93.

In some embodiments, the 5′ homology arm of the first nucleic acid comprises a sequence with at least 90% sequence identity to SEQ ID NO: 96, and the 3′ homology arm of the first nucleic acid comprises a sequence with at least 90% sequence identity to SEQ ID NO: 105.

In some embodiments, the 5′ homology arm of the first nucleic acid comprises a sequence with at least 90% sequence identity to SEQ ID NO: 108, and the 3′ homology arm of the first nucleic acid comprises a sequence with at least 90% sequence identity to SEQ ID NO: 116.

In some embodiments, the 5′ homology arm of the first nucleic acid comprises a sequence with at least 90% sequence identity to SEQ ID NO: 119, and the 3′ homology arm of the first nucleic acid comprises a sequence with at least 90% sequence identity to SEQ ID NO: 127.

In some embodiments, the 5′ homology arm of the first nucleic acid comprises a sequence with at least 90% sequence identity to SEQ ID NO: 130, and the 3′ homology arm of the first nucleic acid comprises a sequence with at least 90% sequence identity to SEQ ID NO: 138.

In some embodiments, the system further comprises a FOXP3 locus-targeting guide RNA (gRNA) comprising a spacer sequence that is complementary to a sequence within the FOXP3 locus, or a fourth nucleic acid encoding the FOXP3 locus-targeting gRNA.

In some embodiments, the 5′ homology arm of the second nucleic acid comprises a sequence with at least 90% sequence identity to SEQ ID NO: 141, and the 3′ homology arm of the second nucleic acid comprises a sequence with at least 90% sequence identity to SEQ ID NO: 149.

In some embodiments, the 5′ homology arm of the second nucleic acid comprises a sequence with at least 90% sequence identity to SEQ ID NO: 152, and the 3′ homology arm of the second nucleic acid comprises a sequence with at least 90% sequence identity to SEQ ID NO: 160.

In some embodiments, the 5′ homology arm of the second nucleic acid comprises a sequence with at least 90% sequence identity to SEQ ID NO: 163, and the 3′ homology arm of the second nucleic acid comprises a sequence with at least 90% sequence identity to SEQ ID NO: 171.

In some embodiments, the 5′ homology arm of the second nucleic acid comprises a sequence with at least 90% sequence identity to SEQ ID NO: 174, and the 3′ homology arm of the second nucleic acid comprises a sequence with at least 90% sequence identity to SEQ ID NO: 183.

In some embodiments, the 5′ homology arm of the second nucleic acid comprises a sequence with at least 90% sequence identity to SEQ ID NO: 186, and the 3′ homology arm of the second nucleic acid comprises a sequence with at least 90% sequence identity to SEQ ID NO: 194.

In some embodiments, the 5′ homology arm of the second nucleic acid comprises a sequence with at least 90% sequence identity to SEQ ID NO: 197, and the 3′ homology arm of the second nucleic acid comprises a sequence with at least 90% sequence identity to SEQ ID NO: 205.

In some embodiments, the 5′ homology arm of the second nucleic acid comprises a sequence with at least 90% sequence identity to SEQ ID NO: 208, and the 3′ homology arm of the second nucleic acid comprises a sequence with at least 90% sequence identity to SEQ ID NO: 217.

In some embodiments, the first nucleic acid is comprised within a first vector.

In some embodiments, the first vector is an adeno-associated virus (AAV) vector.

In some embodiments, the first vector is an AAV vector derived from an AAV of serotype AAV1, AAV2, AAV3, AAV4, AAV5, AAV6, AAV7, AAV8, AAV9, AAV10, or AAV11.

In some embodiments, the second nucleic acid is comprised within a second vector.

In some embodiments, the second vector is an adeno-associated virus (AAV) vector.

In some embodiments, the second vector is an AAV vector derived from an AAV of serotype AAV1, AAV2, AAV3, AAV4, AAV5, AAV6, AAV7, AAV8, AAV9, AAV10, or AAV11.

In some embodiments, the first nucleic acid comprises, between the first 5′ and 3′ homology arms, a nucleotide sequence having at least 95% sequence identity to any one of SEQ ID NOs: 94, 106, 117, 128, and 139.

In some embodiments, the second nucleic acid comprises, between the first 5′ and 3′ homology arms, a nucleotide sequence having at least 95% sequence identity to any one of SEQ ID NOs: 150, 161, 172, 184, 195, 206, and 218.

In some embodiments, the first nucleic acid comprises a nucleotide sequence having at least 95% sequence identity to any one of SEQ ID NOs: 95, 107, 118, 129, and 140.

In some embodiments, the second nucleic acid comprises a nucleotide sequence having at least 95% sequence identity to any one of SEQ ID NOs: 151, 162, 173, 185, 196, 207, and 219.

In some embodiments, one or more of the homology arms is 100-2000 nucleotides in length.

In some embodiments, each of the homology arms is 300-700 nucleotides in length.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 depicts examples of polynucleotides for use in engineering Tregs to insert (i) MND, FKBP-IL2RG, and either a fragment of T1D2 or T1D5-1 TCR with a TRAC hijacking approach, and (ii) MND, FRB-ILRβ, and a naked cytosolic FRB in the FOXP3 locus, for treatment of diabetes. The TRAC hijacking strategy includes knocking out endogenous TCR but using the endogenous TRAC sequence. Cells having both insertions in the two respective loci are referred to as dual-edited cells.

FIG. 2 depicts an editing setup for engineering Tregs with the polynucleotides shown in FIG. 1, and provides the CD4+ T cell donors; AAV constructs; starting cell number used for dual-editing; and the nomenclature for the final product. Mock products were generated using electroporation without addition of AAV donor or nucleases.

FIG. 3 depicts the initial editing rate in cells 3 days after insertion of the polynucleotides as shown in FIG. 1 have been inserted. CD4+ T cells from three donors (two T1D subjects and one healthy donor) were dual-edited using RNPs targeting TRAC andFOXP3 loci, respectively, in association with delivery of either 1) VIN 10019-Genti 122 AAV T1D5-1+3362 AAV or 2) VIN 10020-Genti 122 AAV T1D2+3362 AAV to generate hT1D5-1 EngTregs and hT1D2 EngTregs, respectively. The drawing shows the high level of initial dual-editing achieved in this study. Dual-edited cells are present in the upper-right quadrant of each flow plot. Initial dual-editing rates ranged from 10.1% to 18.9% based on co-expression of CD3+/HA+ as measured by FACS analysis. In dual-edited cells, CD3 expression is restored by successful introduction of the islet antigen-specific TCR (isTCR) into the TRAC locus, and HA staining indicates successful expression of HA-tagged endogenous FoxP3 following HDR editing of the FOXP3 locus.

FIG. 4 depicts an example protocol for engineering Treg cells with expansion of dual-edited cells using rapamycin. Three days after editing, cells were seeded in 10 nM Rapamycin for 12 days of expansion, followed by repeat anti-CD3/CD28 bead stimulation on day 12. The total number of cells seeded are listed for each donor/TCR and ranged between 1.99×10⁶-2.72×10⁶.

FIG. 5 shows enrichment of dual-edited cells 15 days after introducing into the cells the polynucleotides as shown in FIG. 1. The percentage of double positive CD3+/HA-FoxP3+ EngTregs at day 19 ranged from 81.1% to 89.2% demonstrating enrichment of the dual-positive T1D2+/FoxP3+ and T1D5-1+/FoxP3+ cells in both donors having T1D and healthy control donor. The results indicate that: 1) T1D2+/FoxP3+ and T1D5-1+/FoxP3+ double-positive EngTreg cells can be enriched in rapamycin as expected and 2) cells from donors having T1D can be enriched in a similar manner as cells from a healthy control donor.

FIG. 6A and FIG. 6B show suppression of activated Teff cells by engineered Tregs made by dual-editing, including insertion of the polynucleotides as shown in FIG. 1 to produce Tregs expressing T1D2 (FIG. 6A) or T1D5-1 (FIG. 6B) TCRs. Teff cells were activated by either anti-CD3/CD28, or cognate IGRP₃₀₅₋₃₂₄ peptide in the presence of myeloid dendritic cells (mDCs) as antigen-presenting cells (APCs). Both T1D2-expressing and T1D5-1 dual-edited EngTregs from either donor exhibited robust suppressive activity (>80% suppression) against Teff cells targeting the identical IGRP peptide, as summarized in bar graphs at bottom.

FIG. 6C shows results from a polyclonal islet suppression assay that was developed and performed to assess the capacity of Ag-specific dual-edited EngTregs to manifest bystander suppression. This assay uses a pool of autologous Teff cells (derived from the same subjects having T1D) activated in vitro using APCs (mDCs) pulsed with a pool of islet peptides derived from 4 major islet antigens, including IGRP, GAD65, PPI, and ZNT8.

T1D2- or T1D5-1-expressing EngTregs generated via a combination of targeted FOXP3 and TRAC locus editing were generated for comparison to Tregs engineered via lentiviral (LV) delivery of a sequence encoding the same islet-specific TCR (T1D2 or T1D5-1, respectively). These LV-edited T1D2+/FoxP3+ and T1D5-1+/FoxP3+ edited cells differed from the EngTregs dual-edited at TRAC and FOXP3 loci in multiple ways, including: (i) the islet-specific TCR expressed by LV-edited cells contained a murine, not human, TCRβ chain; (ii) LV-edited cells expressed an intact endogenous TDR; and (iii) LV-edited cells did not express components of a chemically induced signaling complex (CISC) for IL-2 signal transduction in the presence of rapamycin. The results show that the murine LV-edited mT1D2+/FoxP3+ and mT1D5-1+/FoxP3+ edited cells suppressed the proliferation of a mixed population of Teff cells stimulated by a pool of islet peptides at multiple TeffDC ratios. The left graph shows % suppression and the right graph shows the % suppression when normalized by an anti-CD3/CD28 bead assay. These finding support the concept that islet-specific EngTregs can mediate bystander suppression of autologous, islet-specific Teff present in T1D subjects.

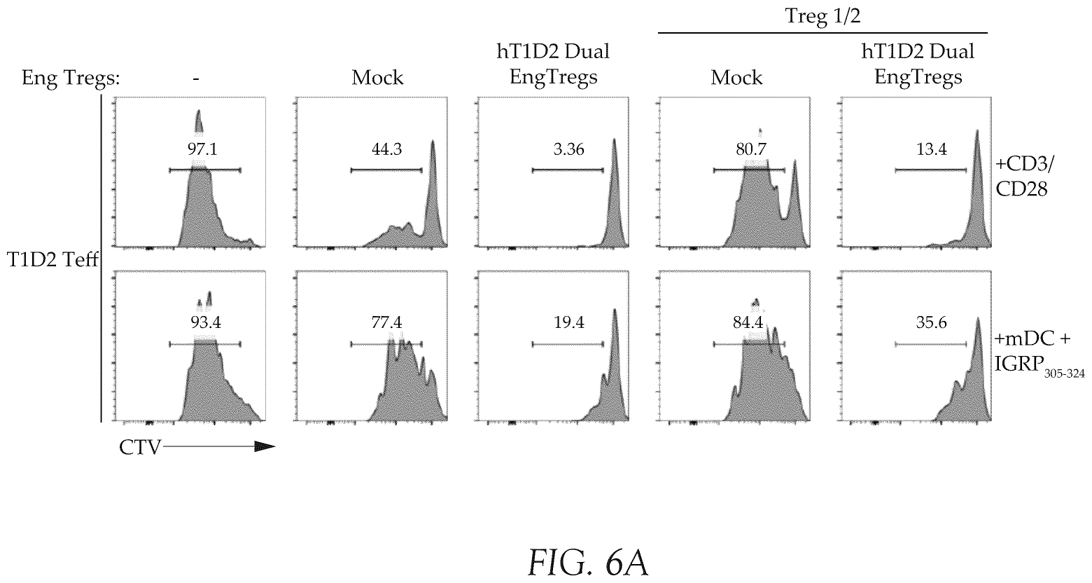

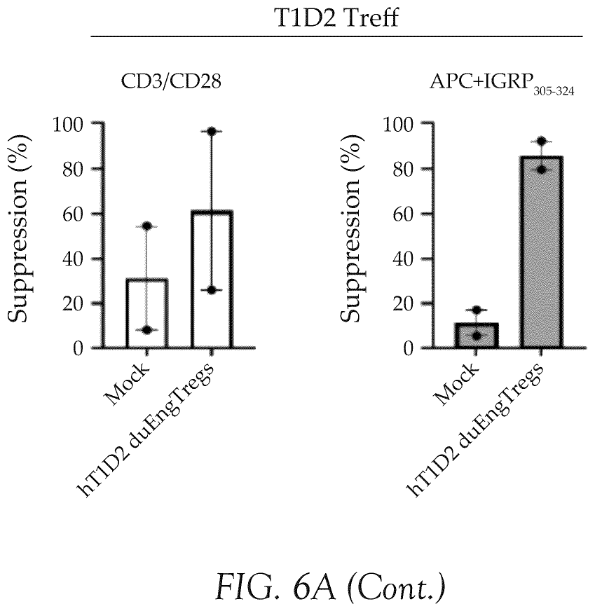

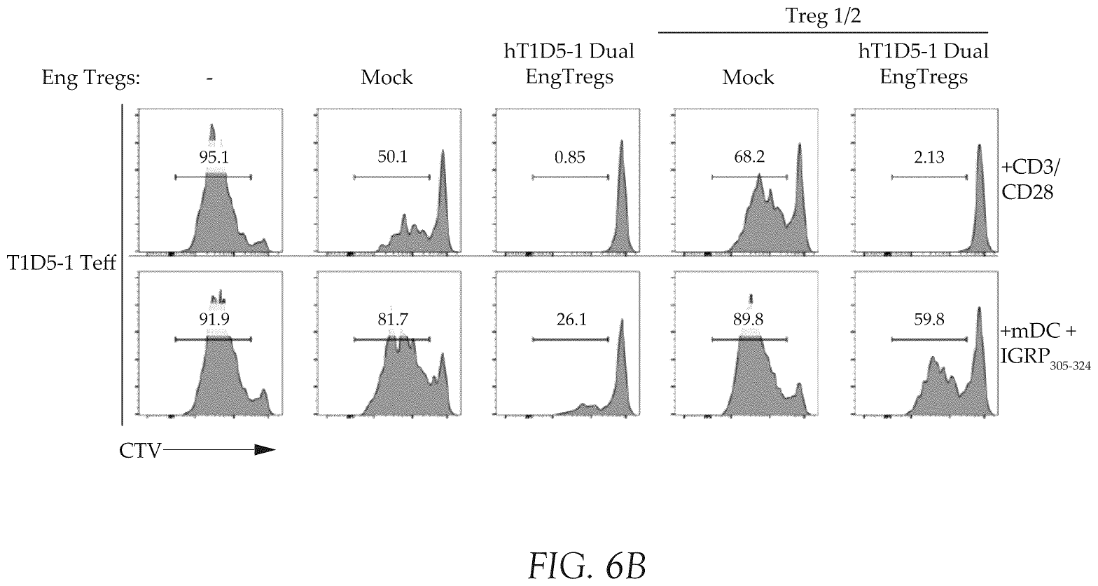

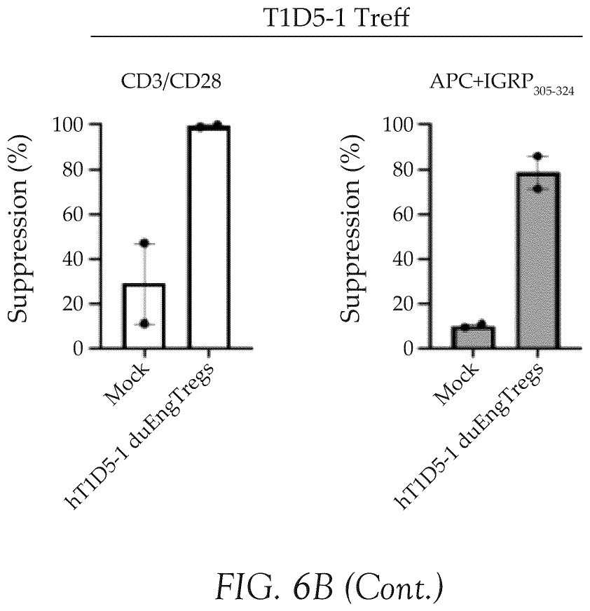

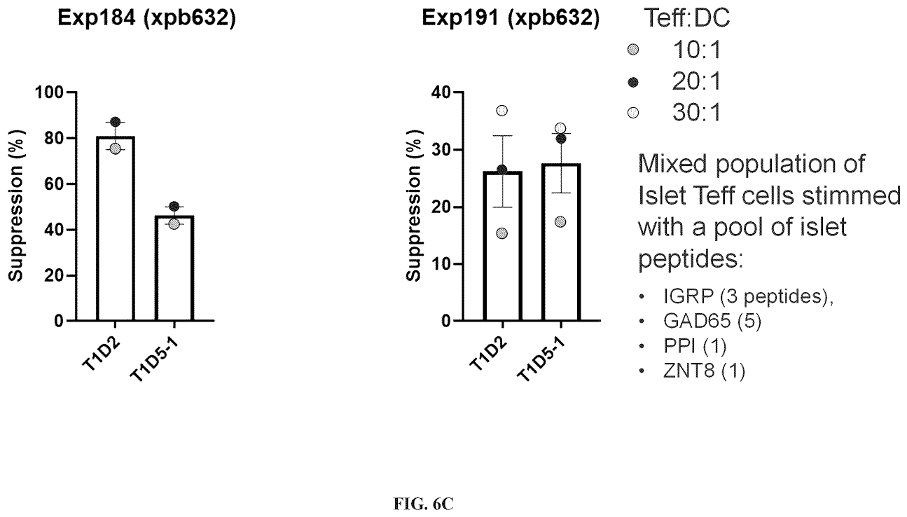

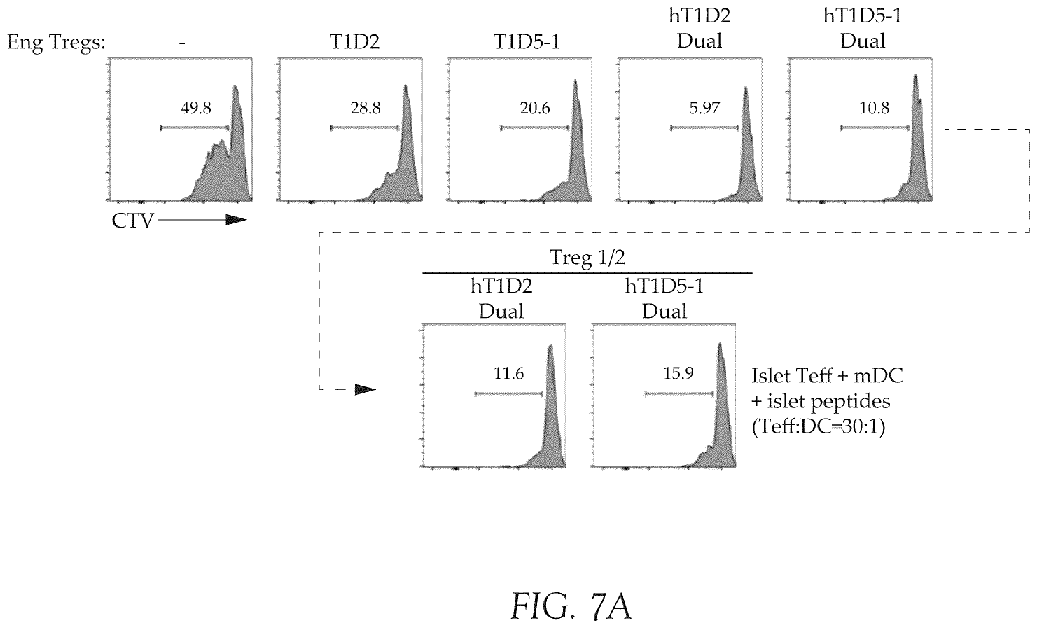

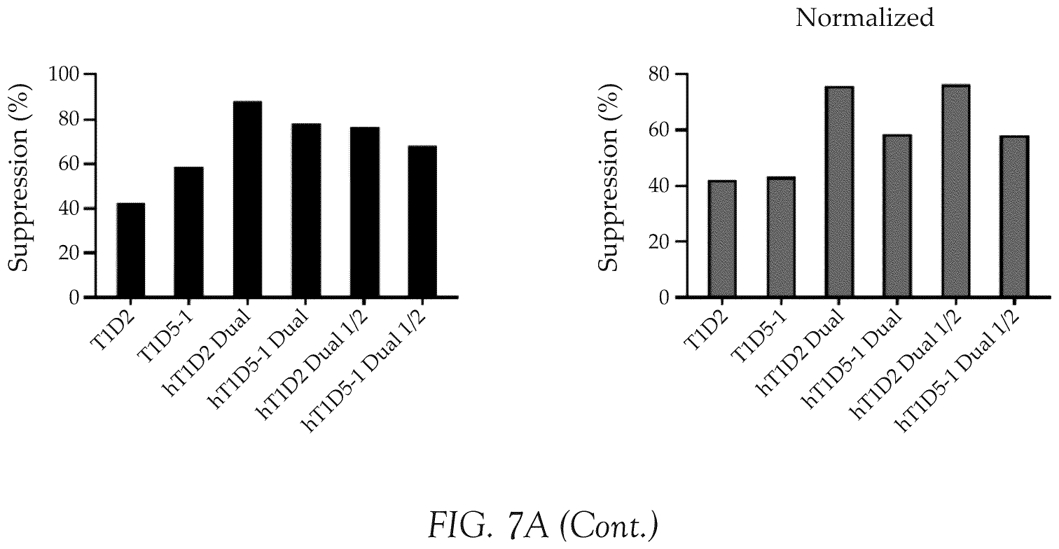

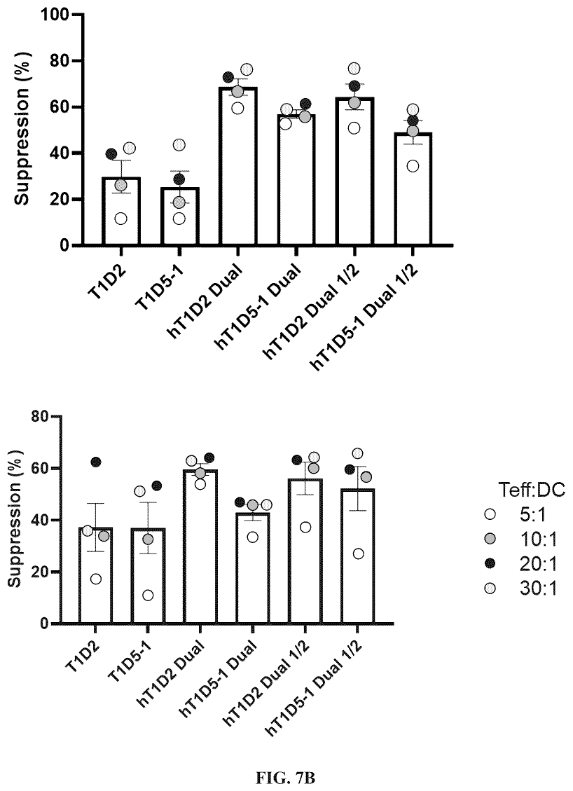

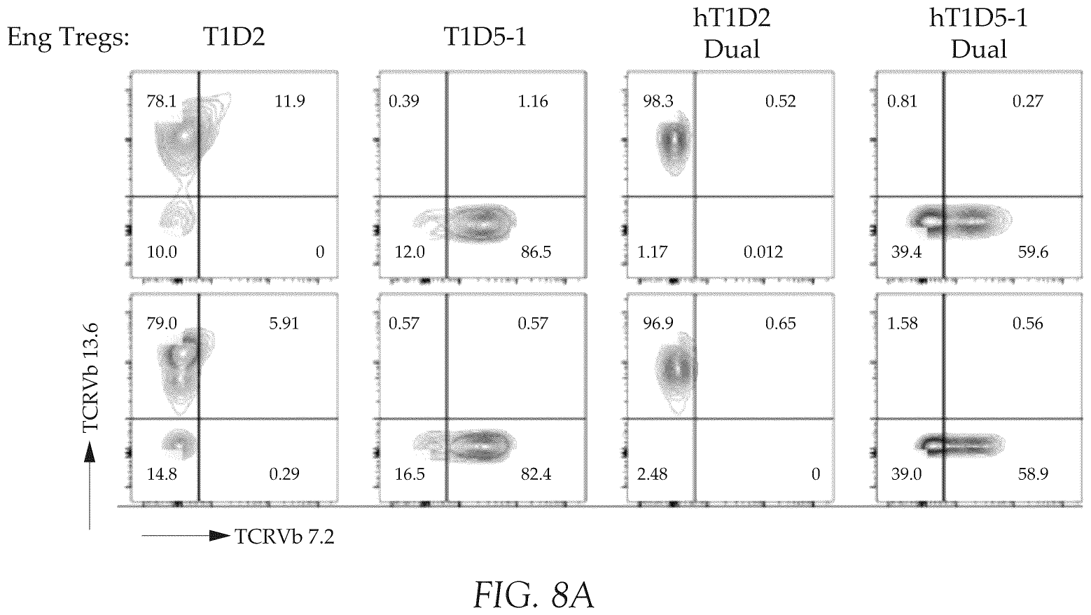

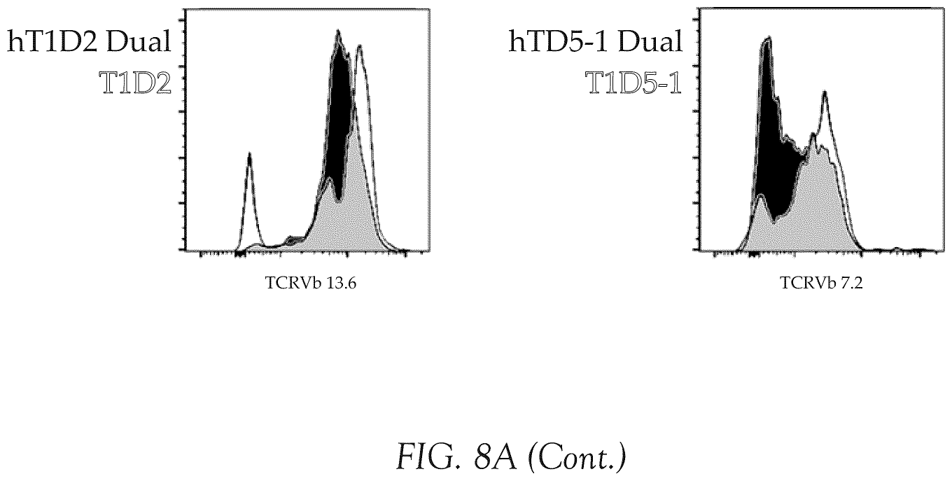

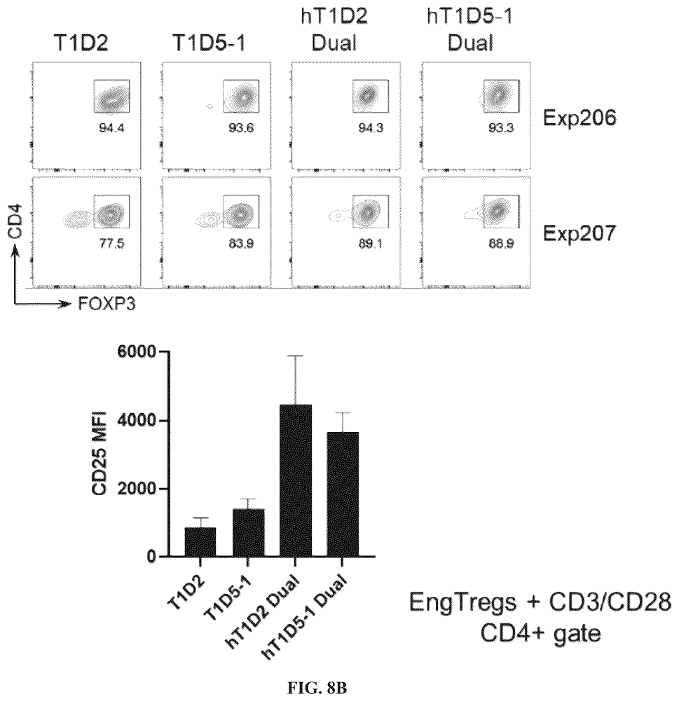

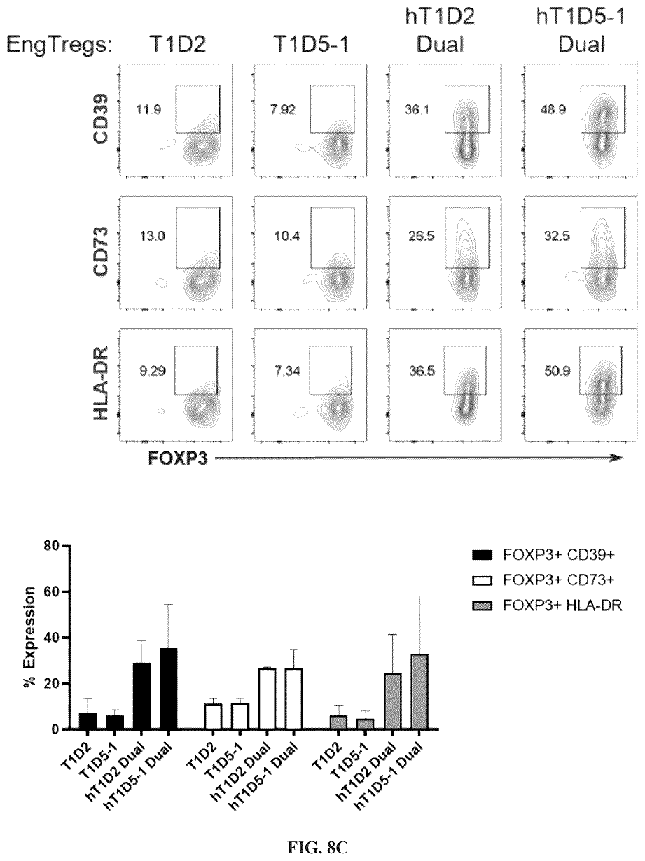

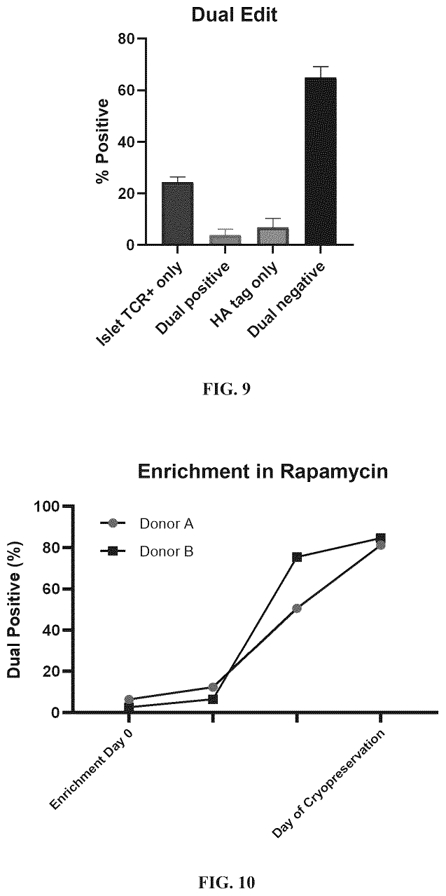

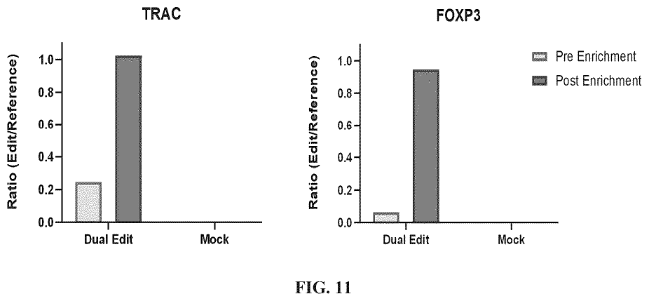

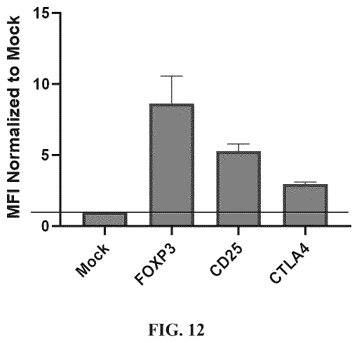

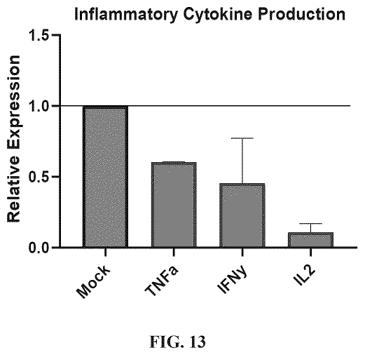

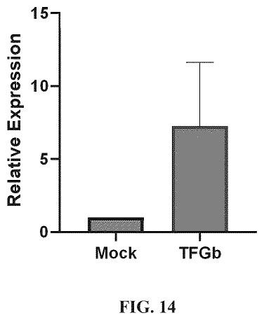

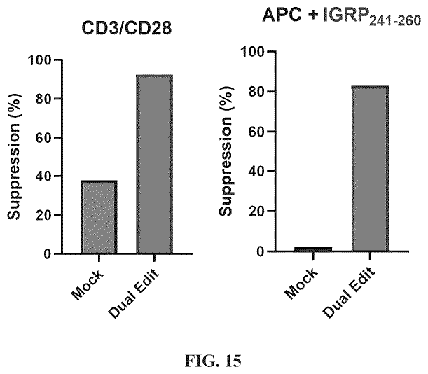

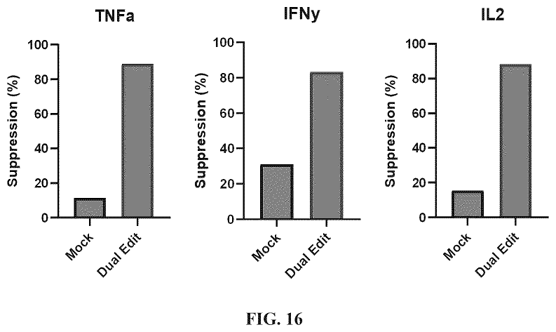

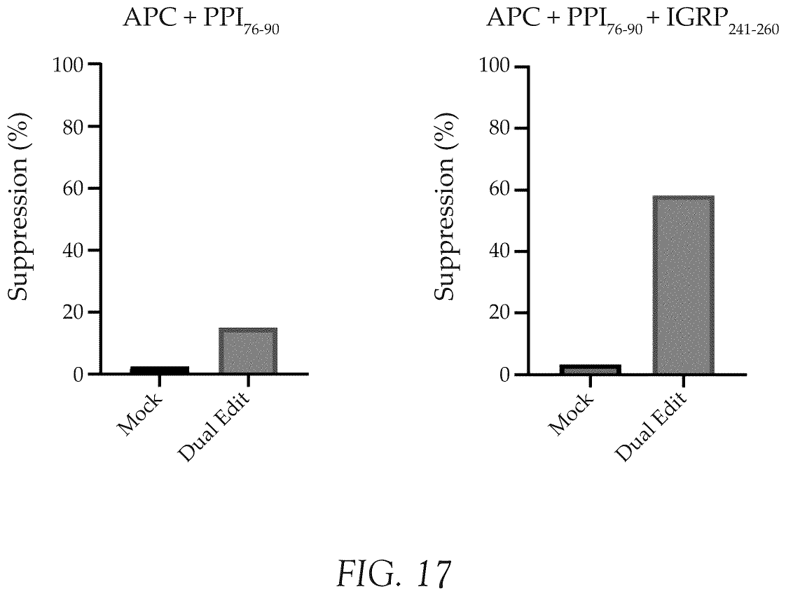

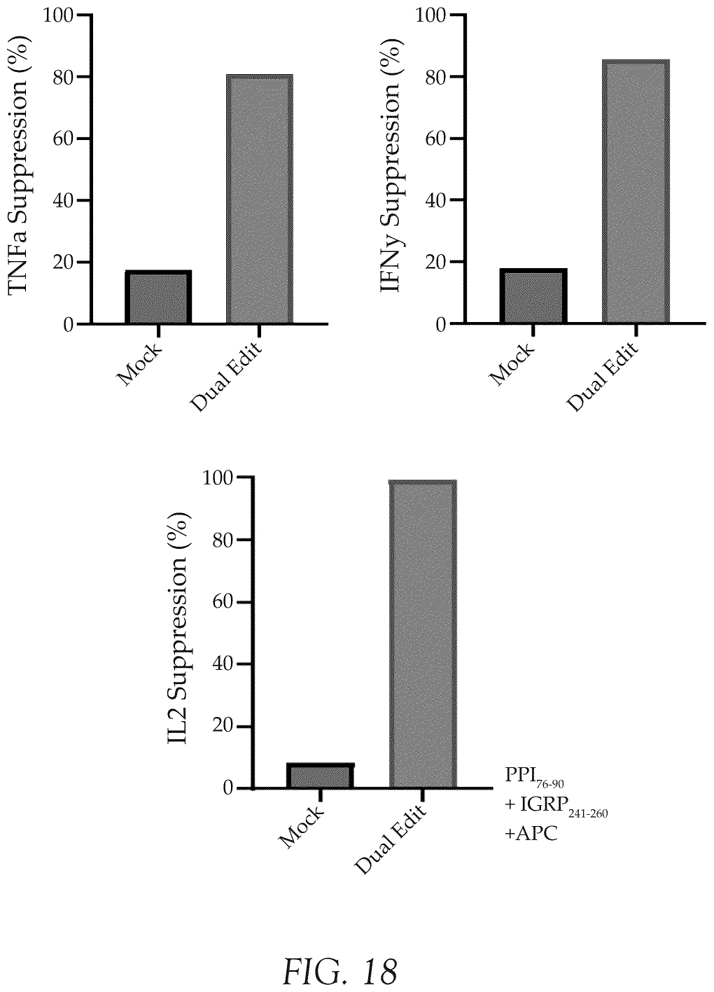

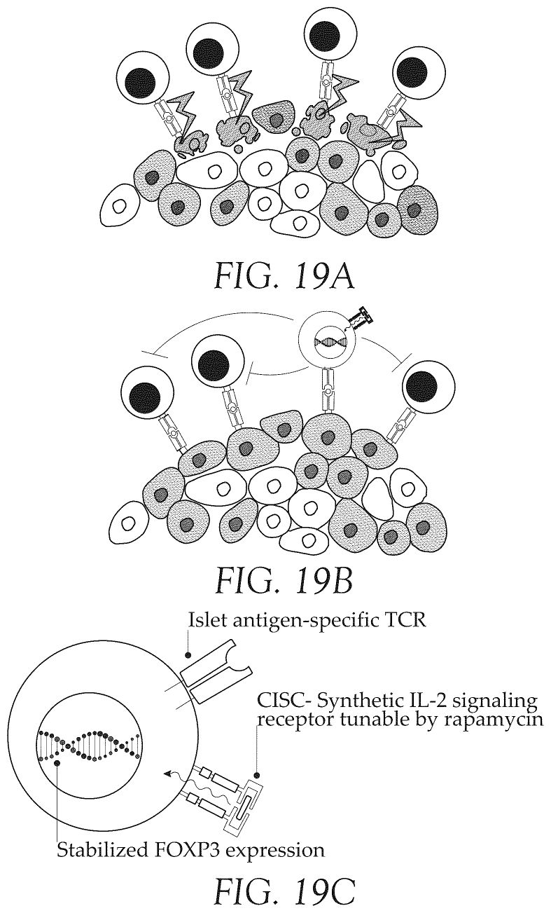

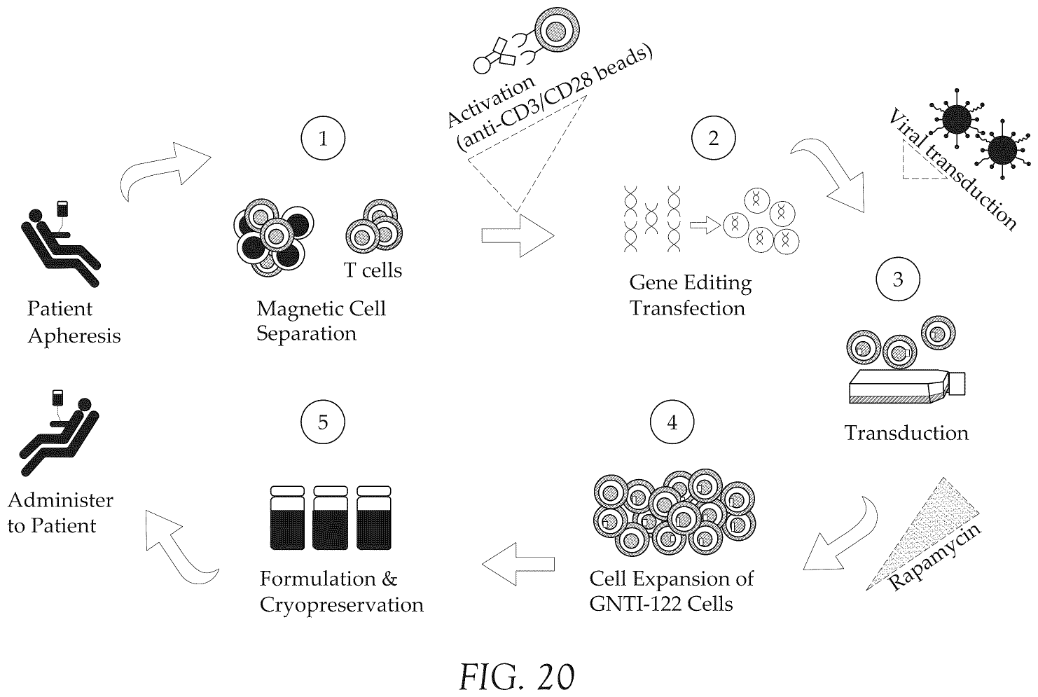

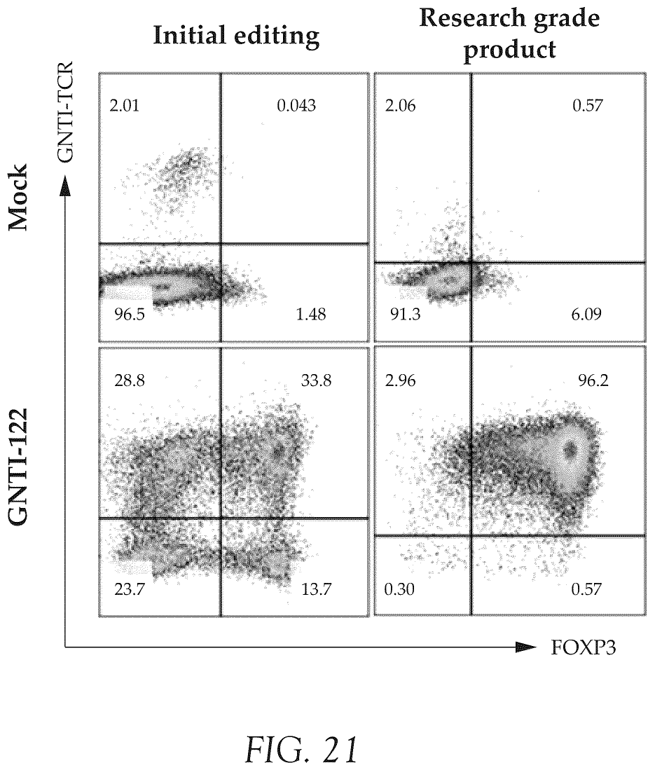

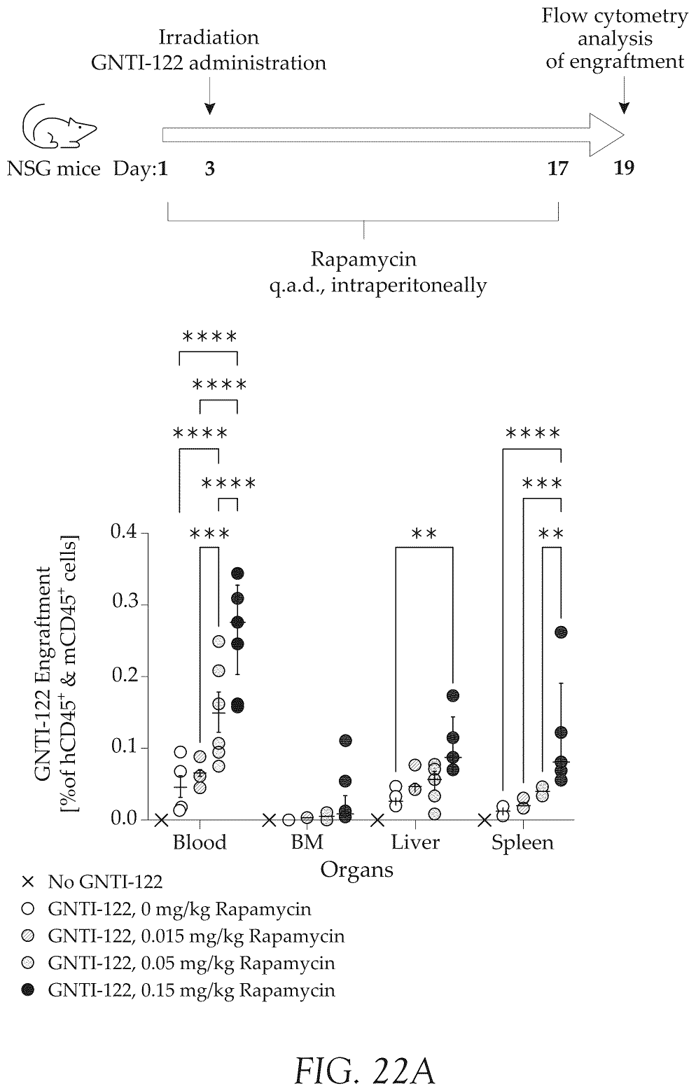

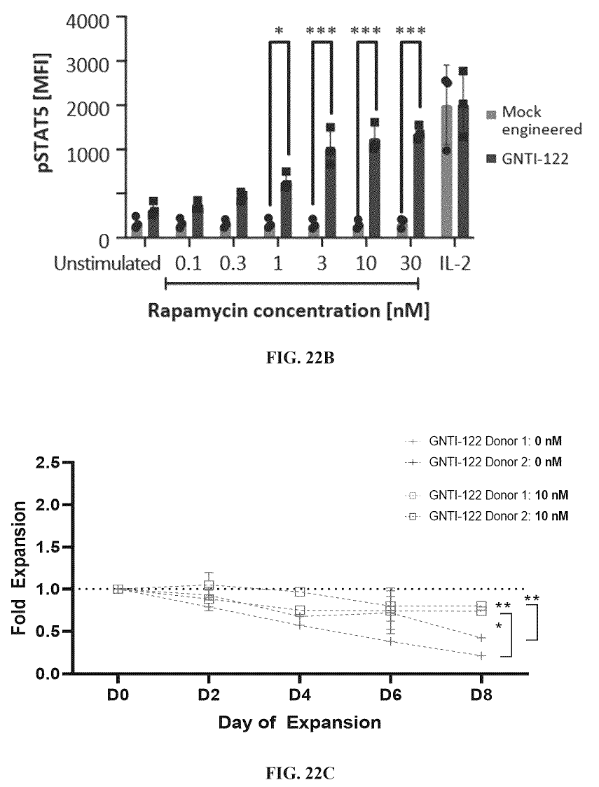

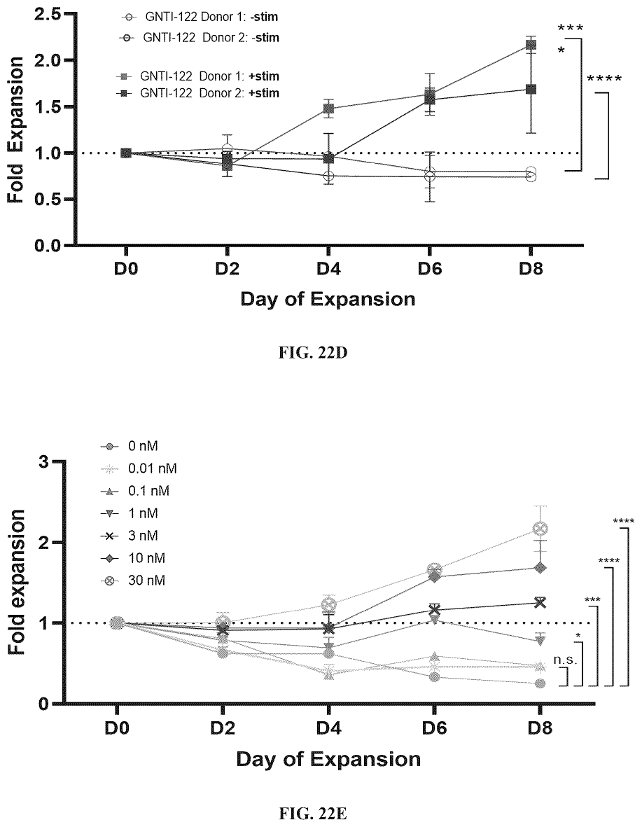

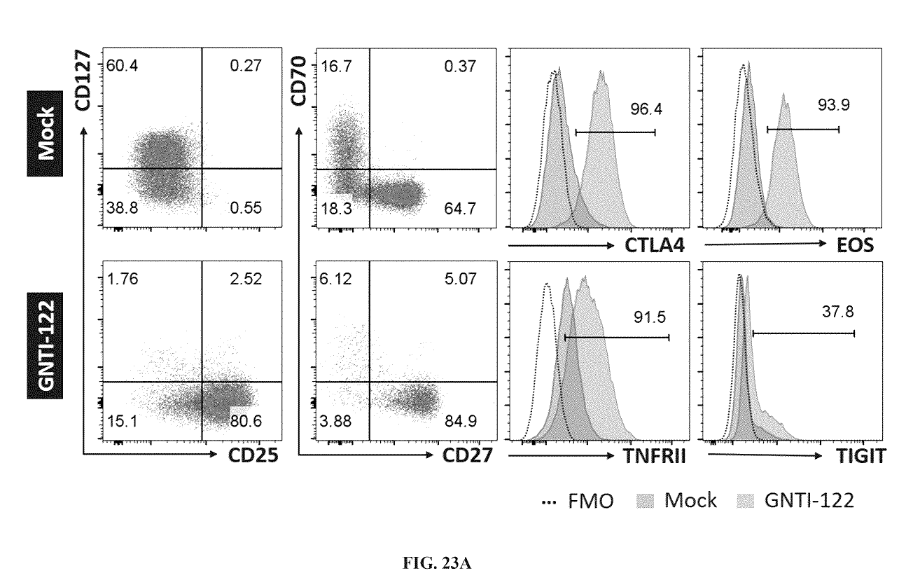

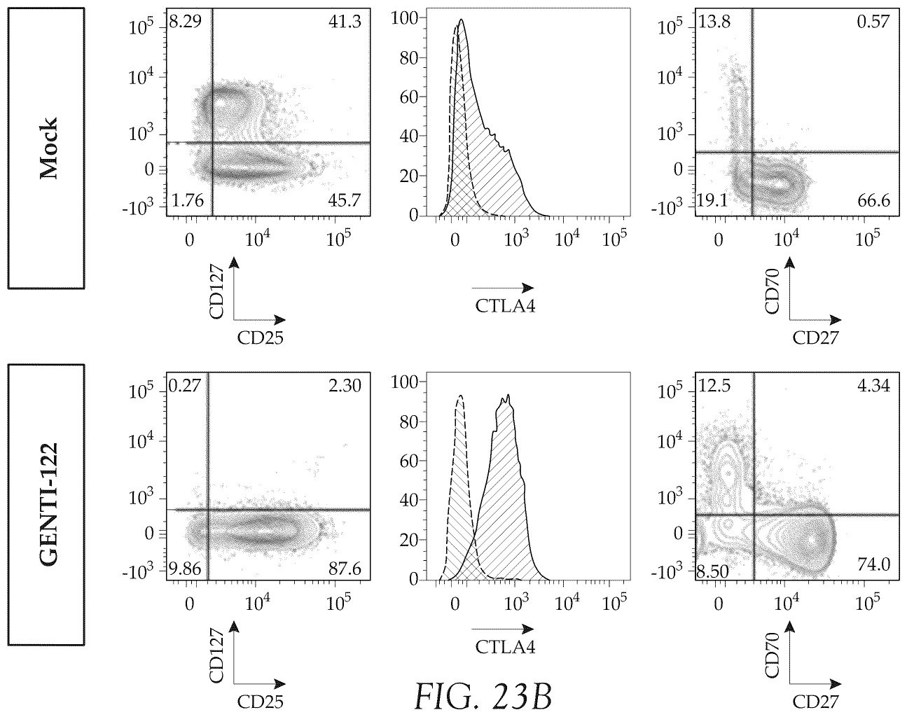

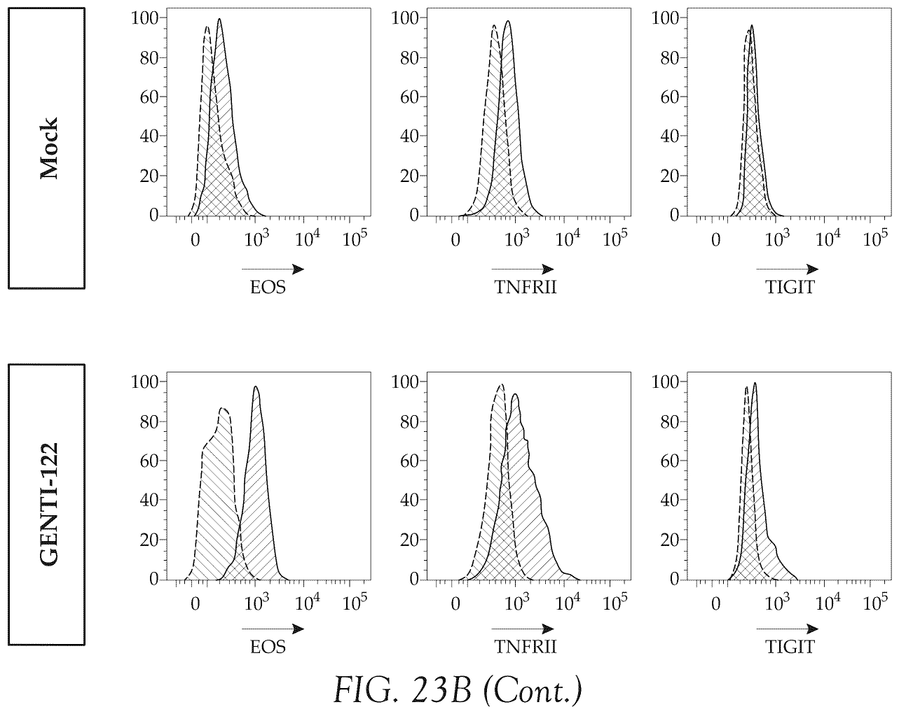

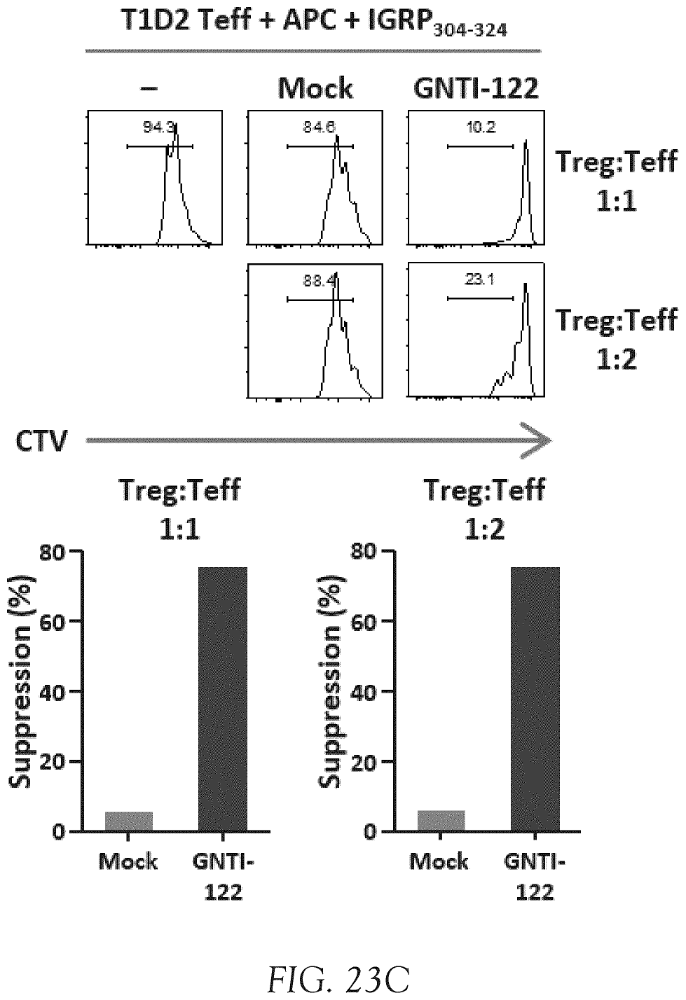

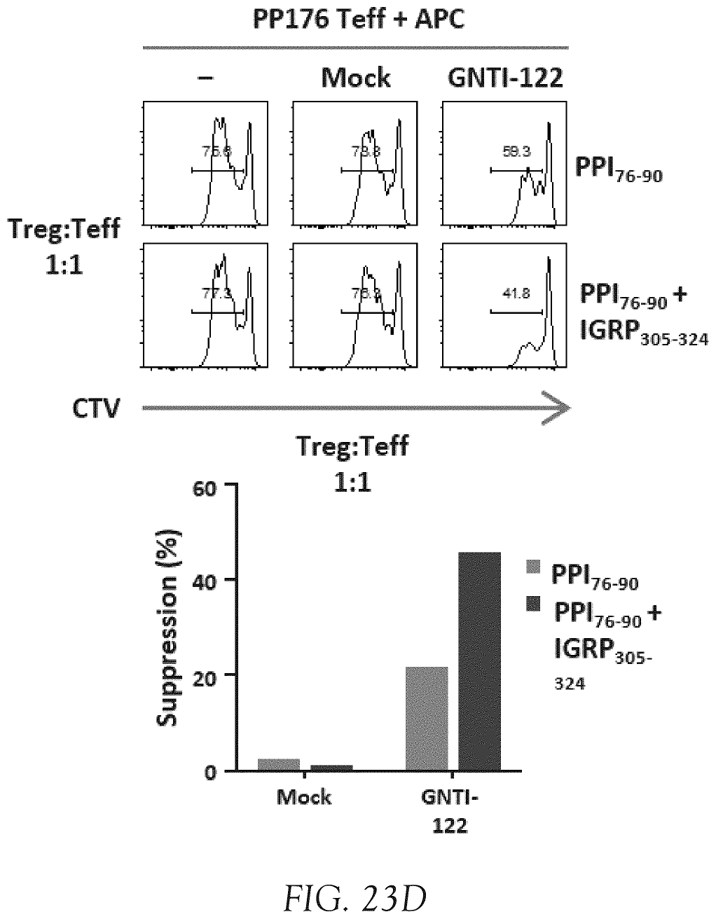

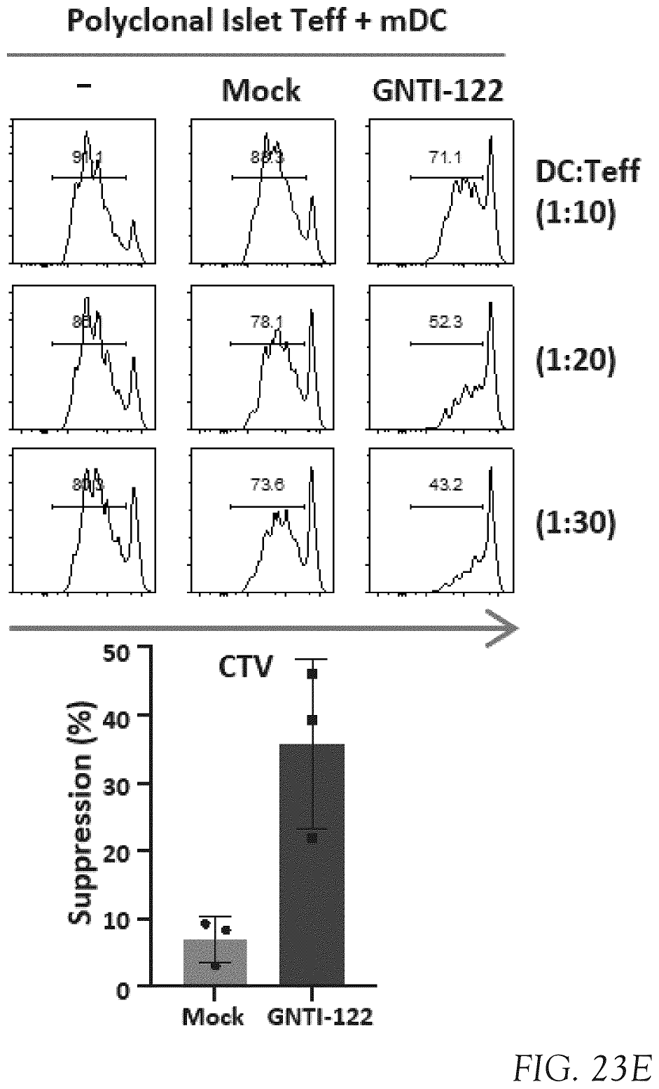

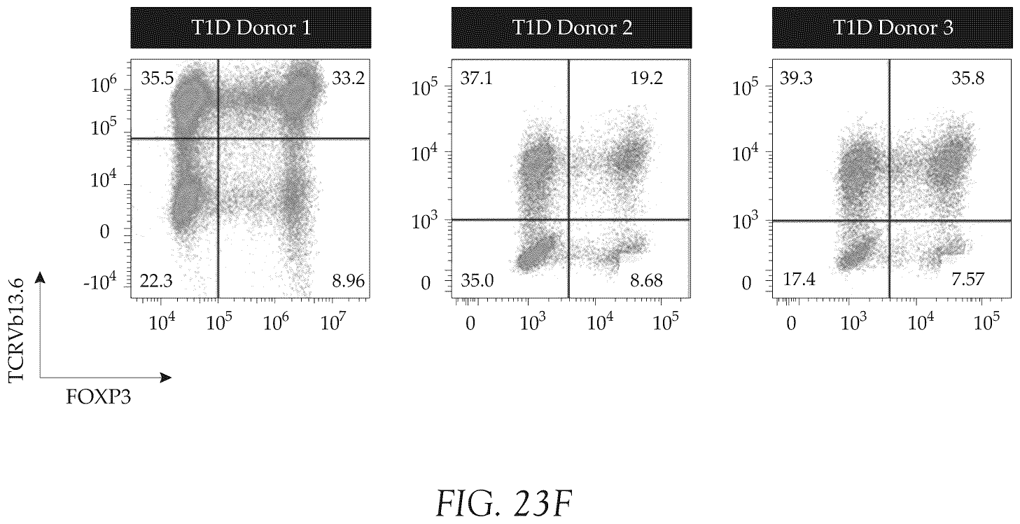

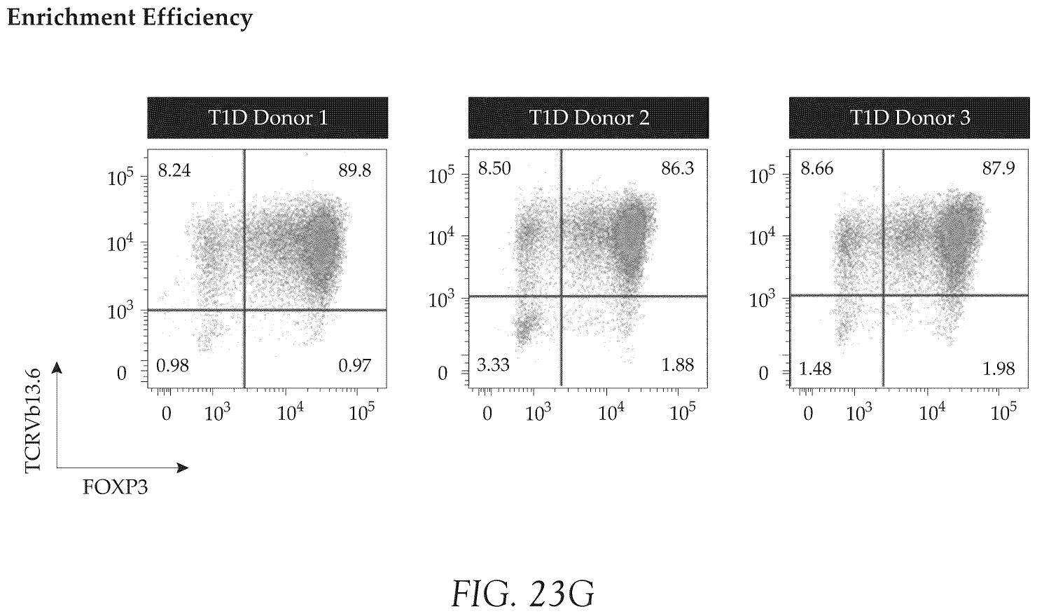

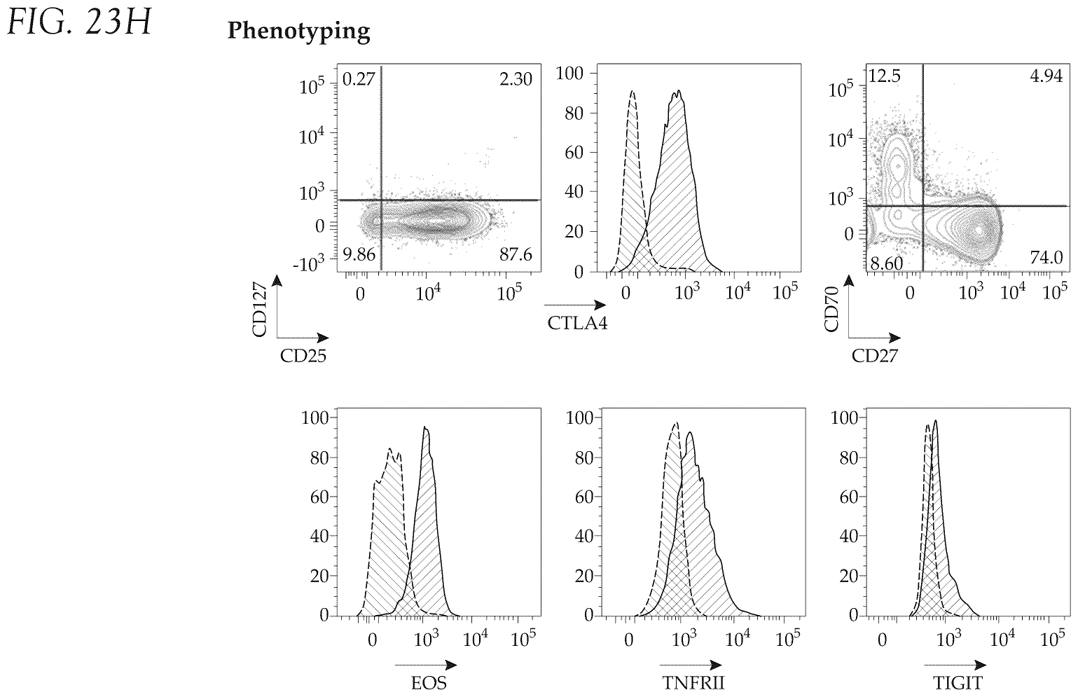

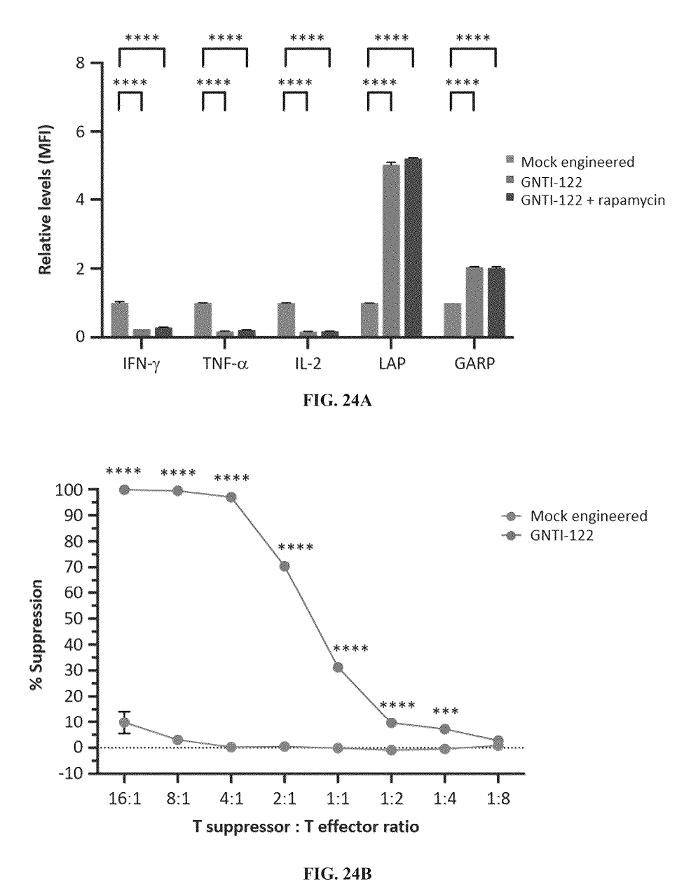

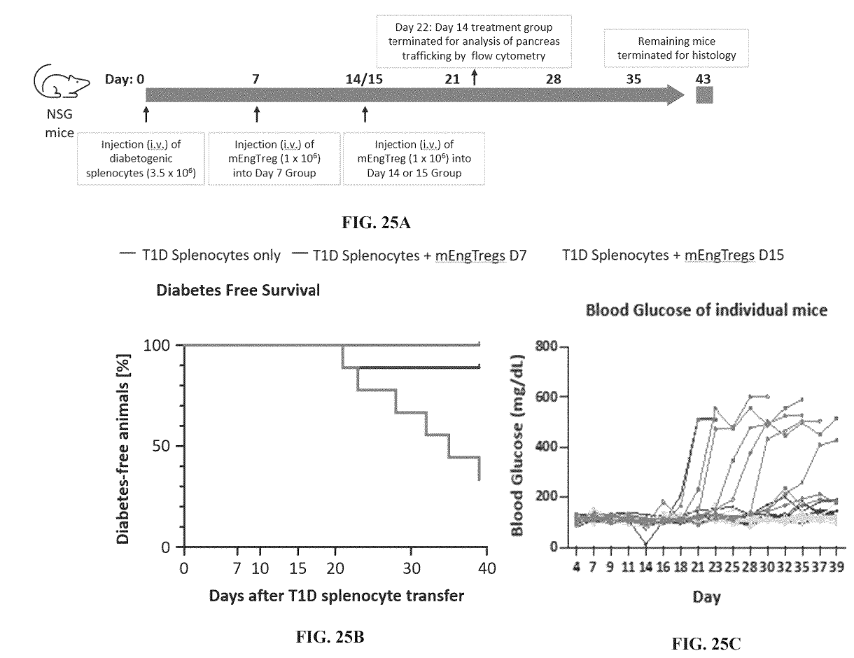

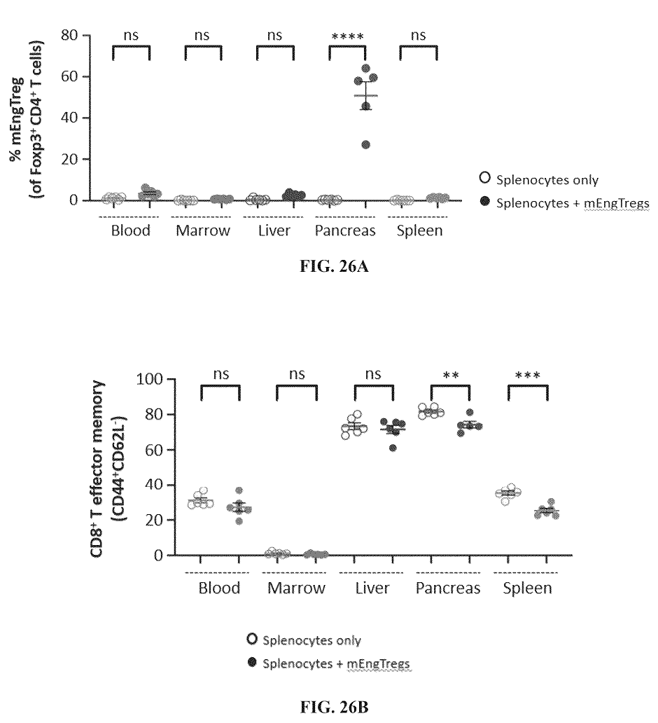

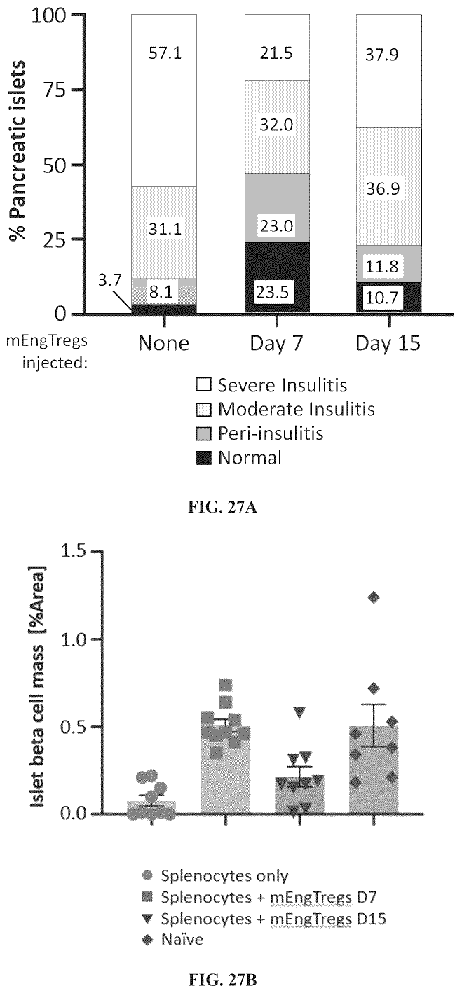

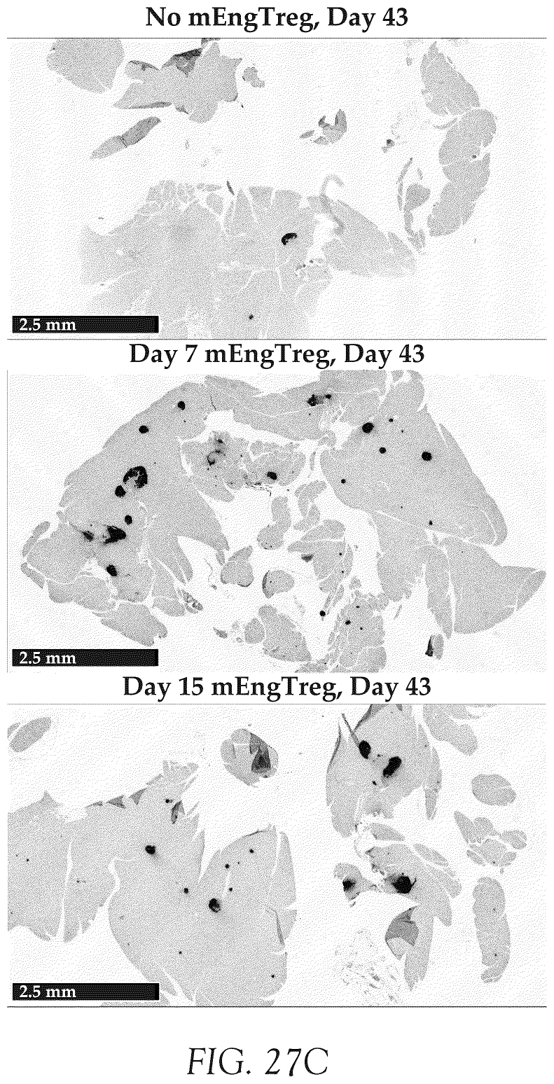

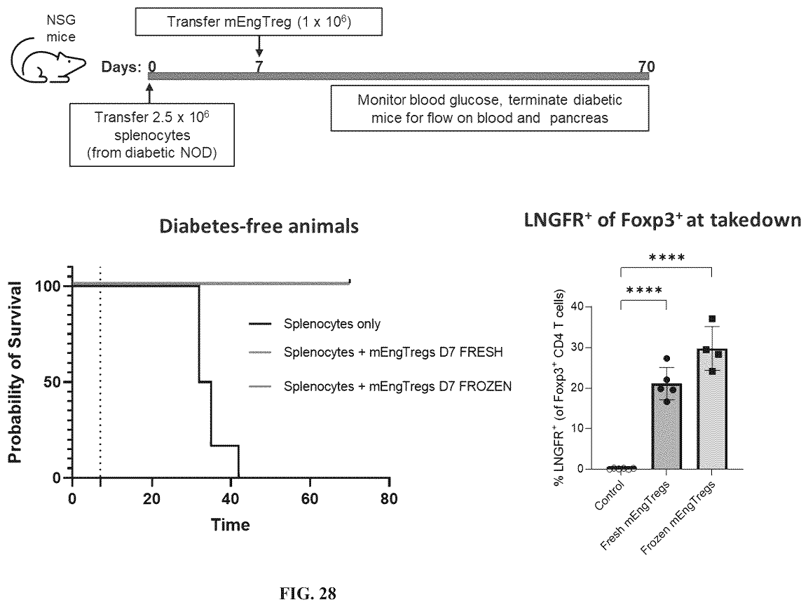

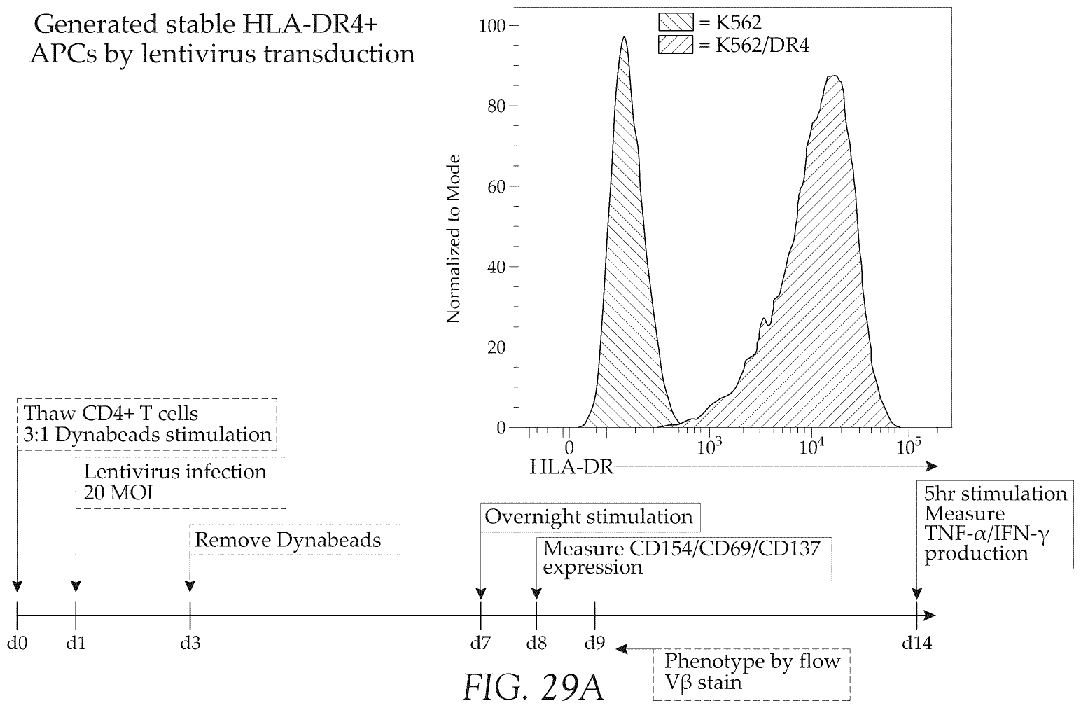

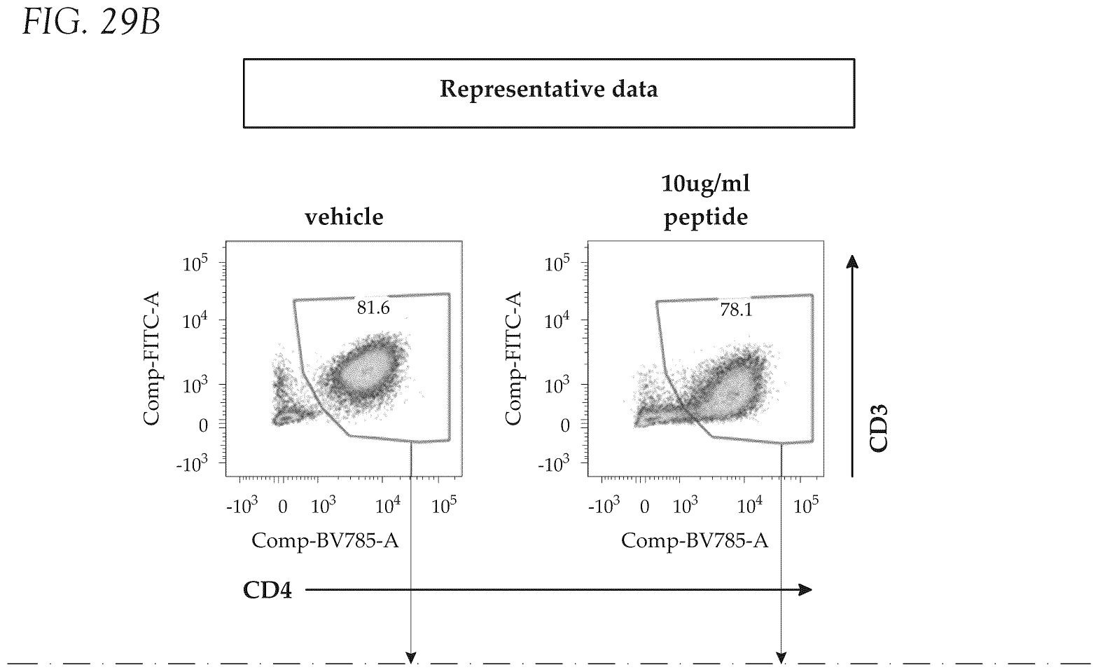

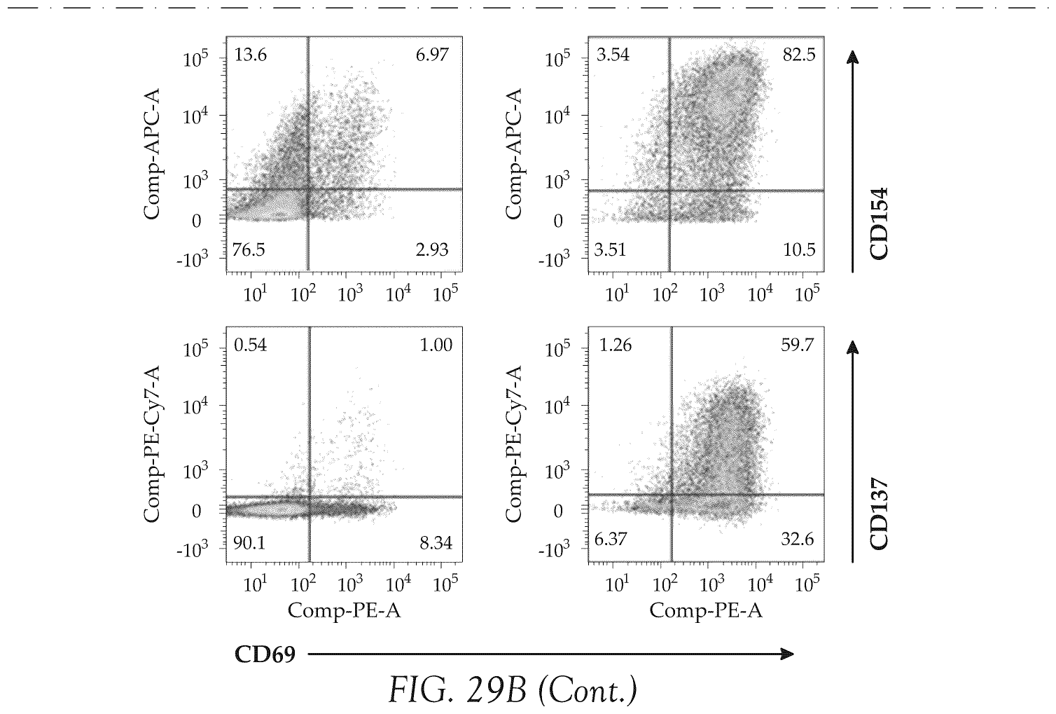

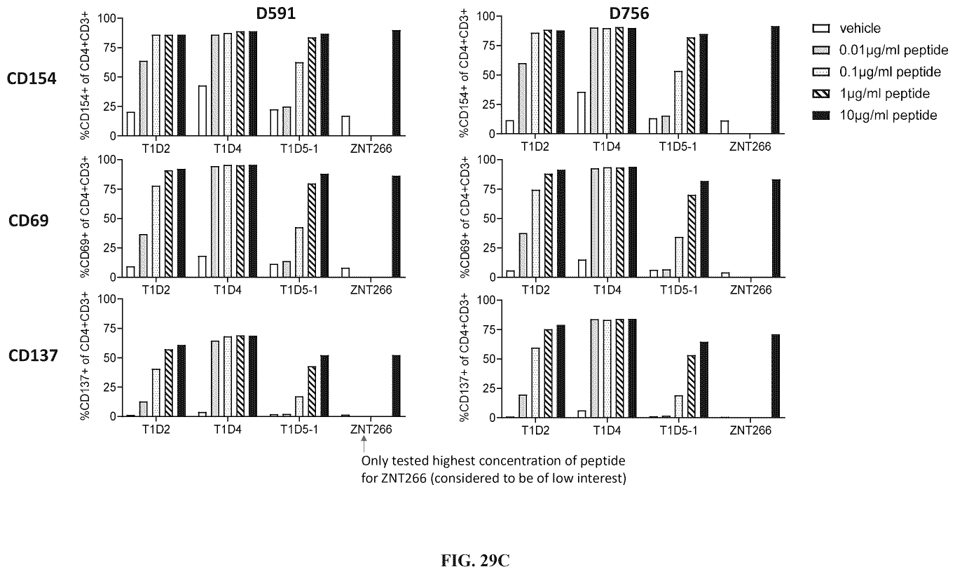

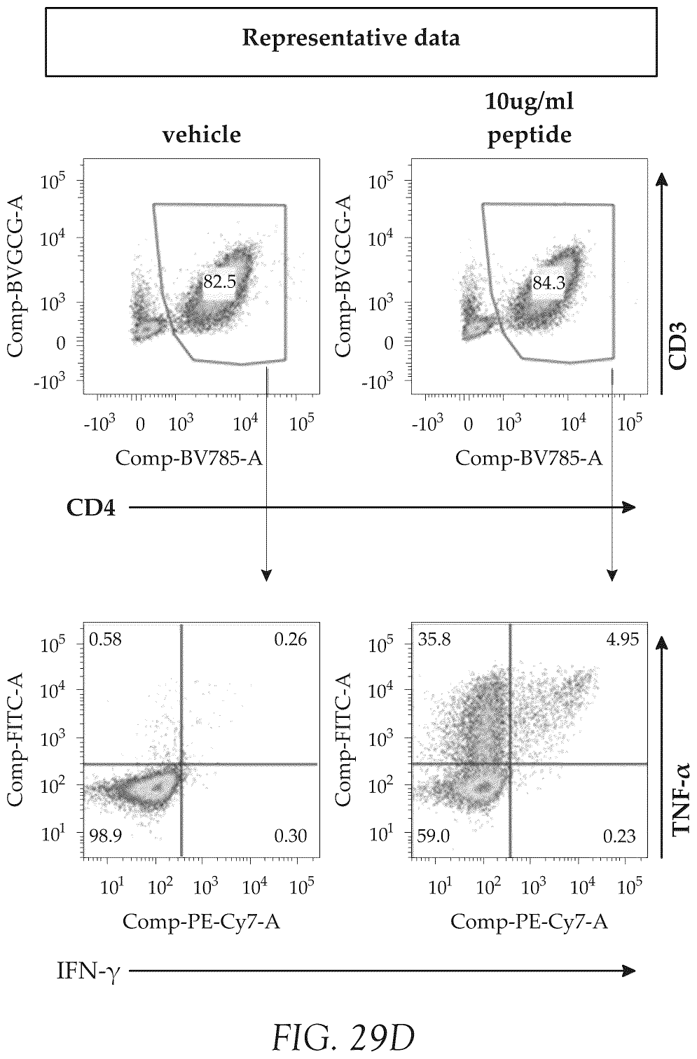

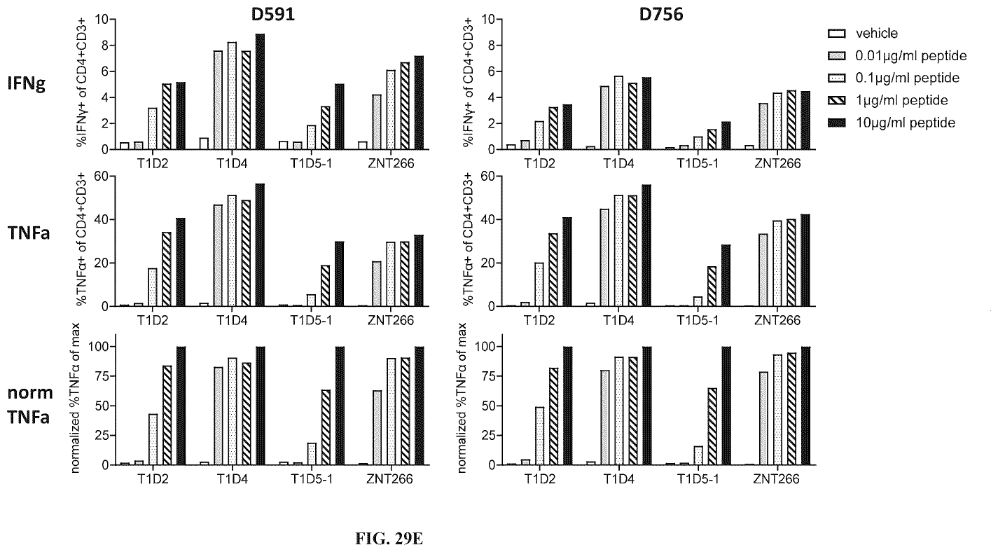

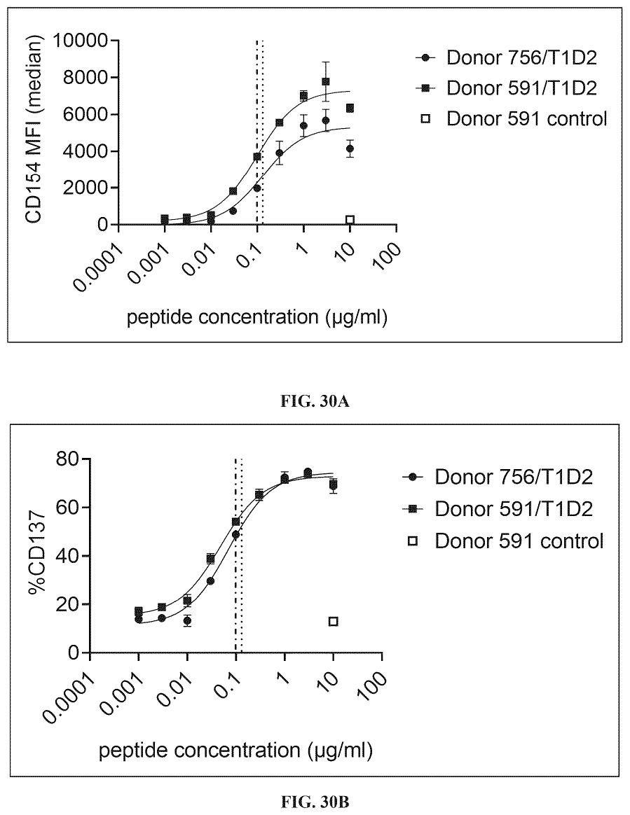

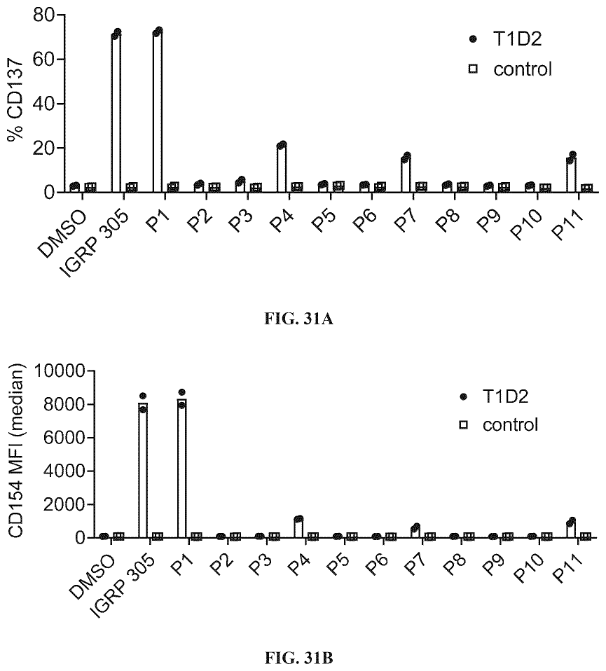

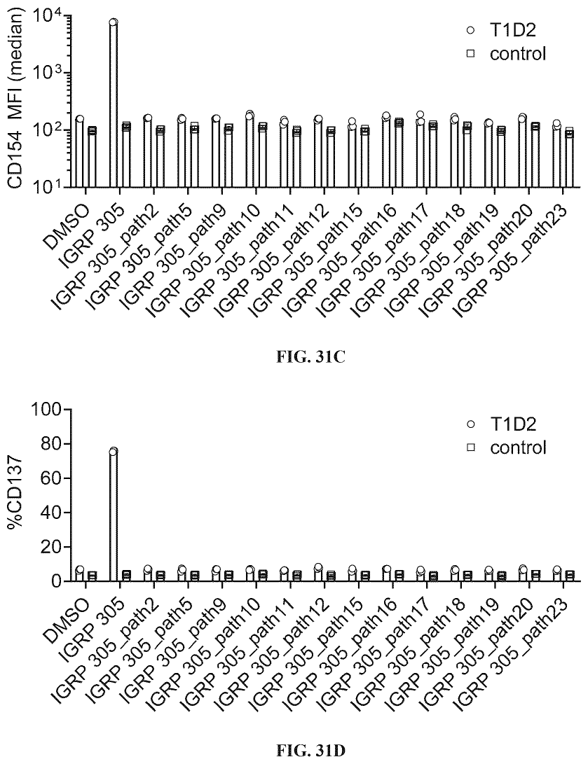

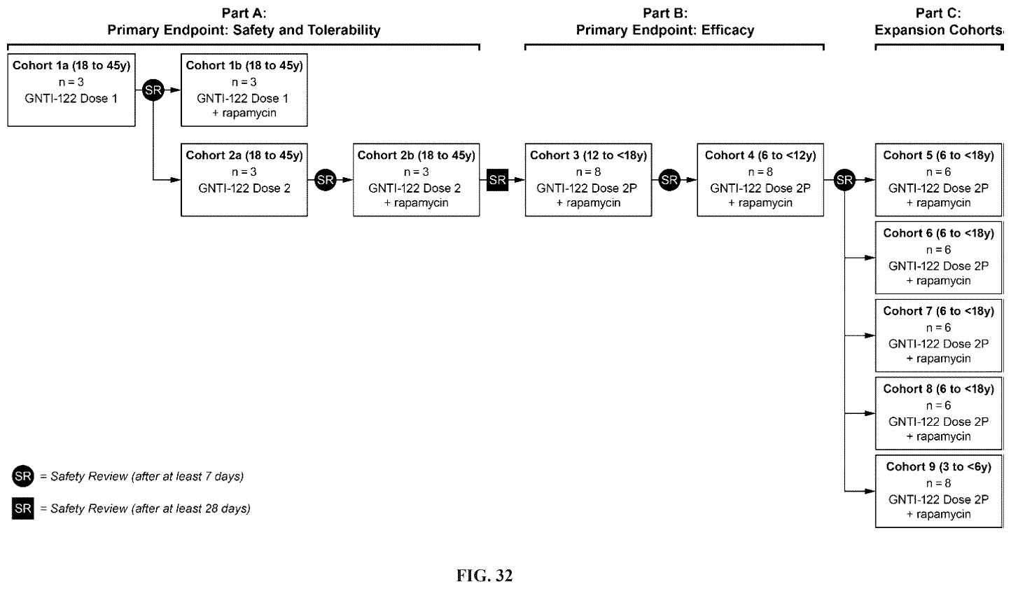

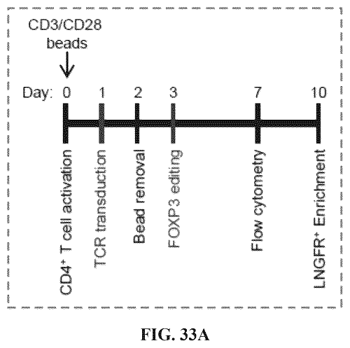

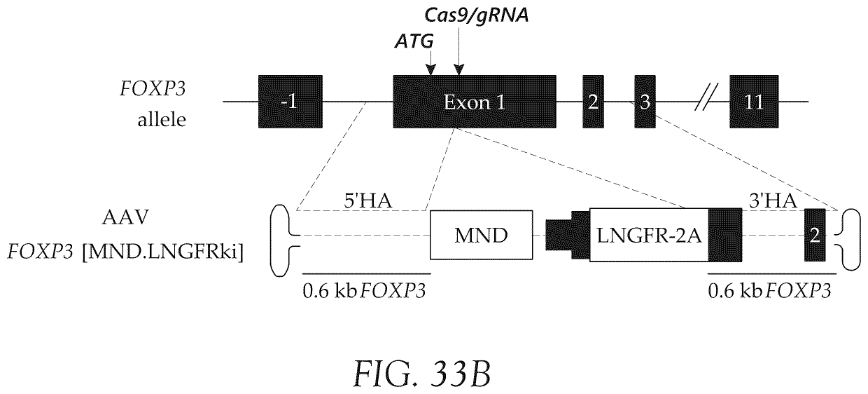

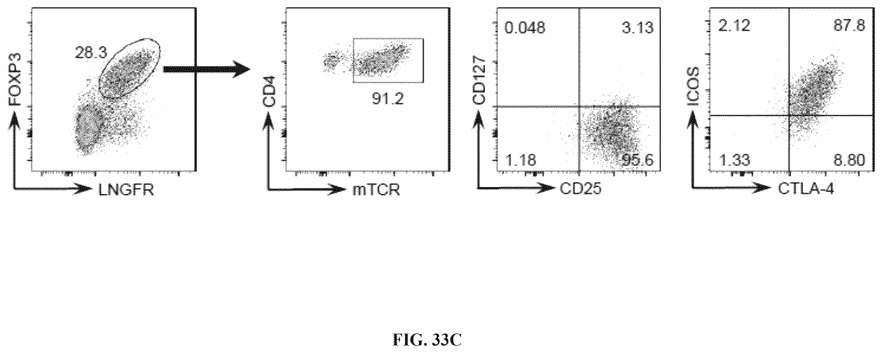

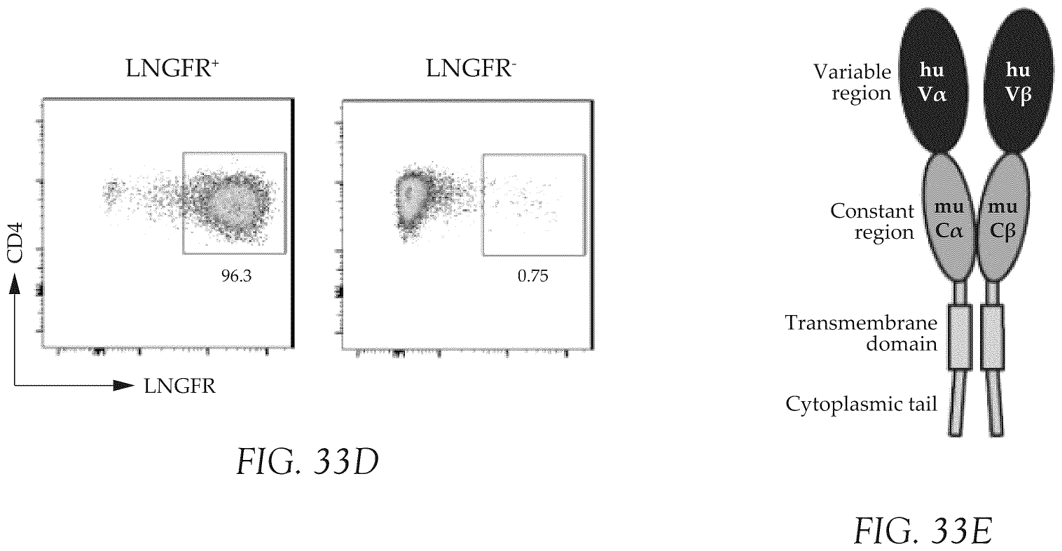

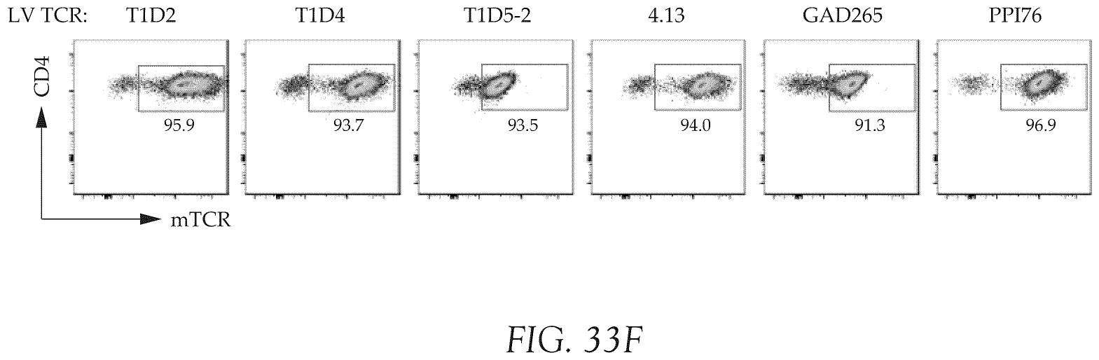

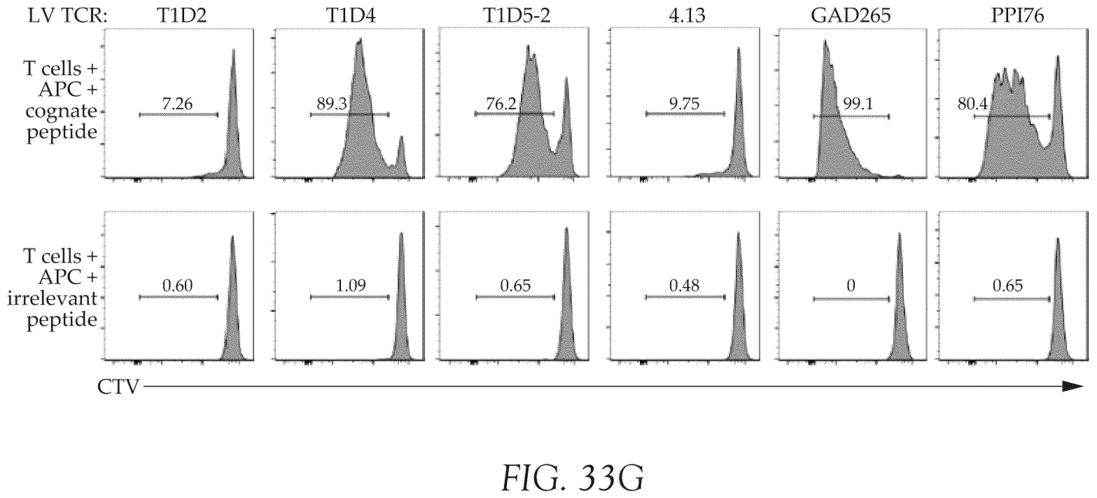

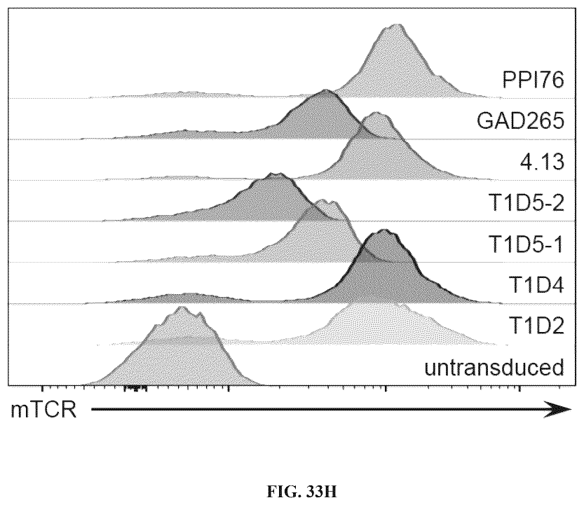

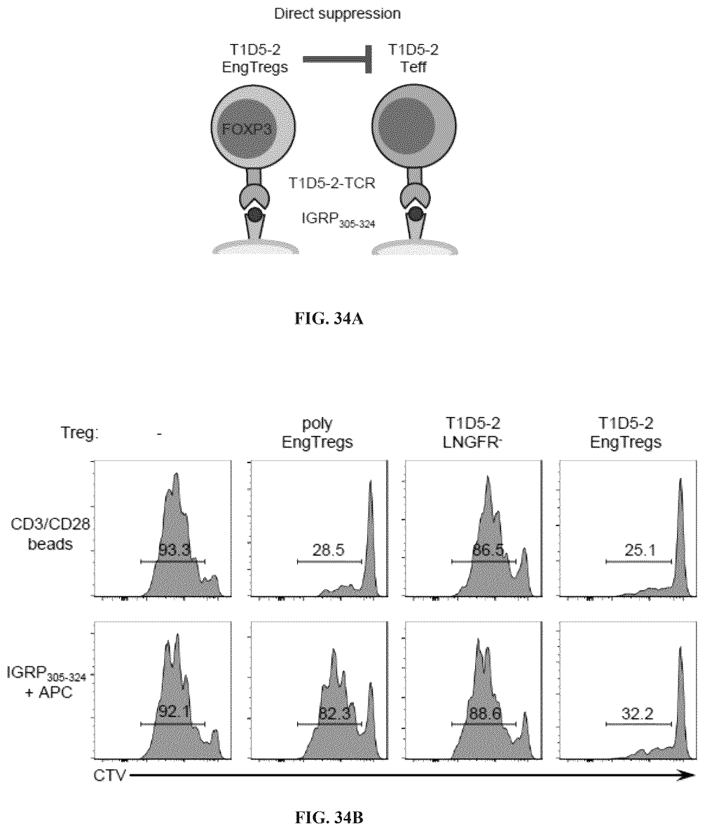

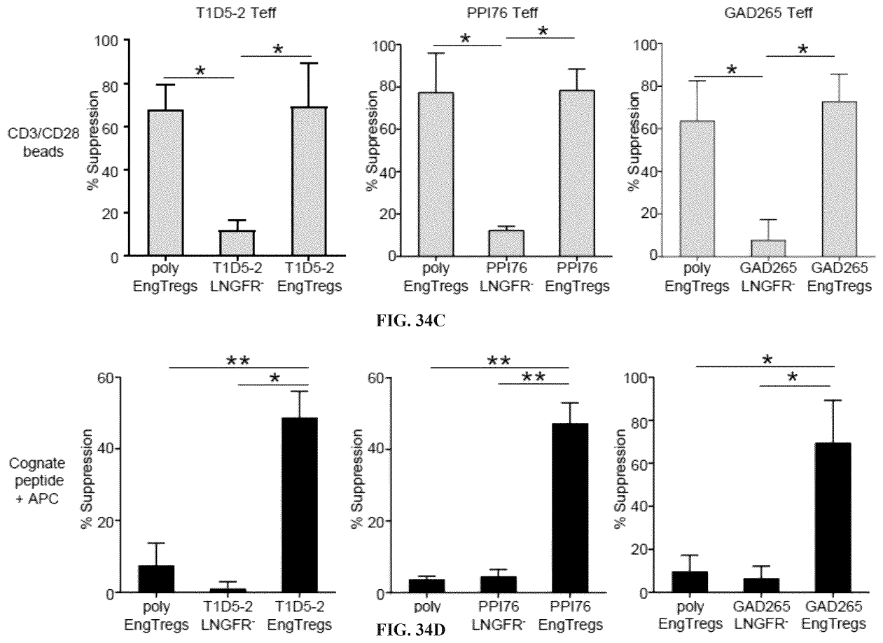

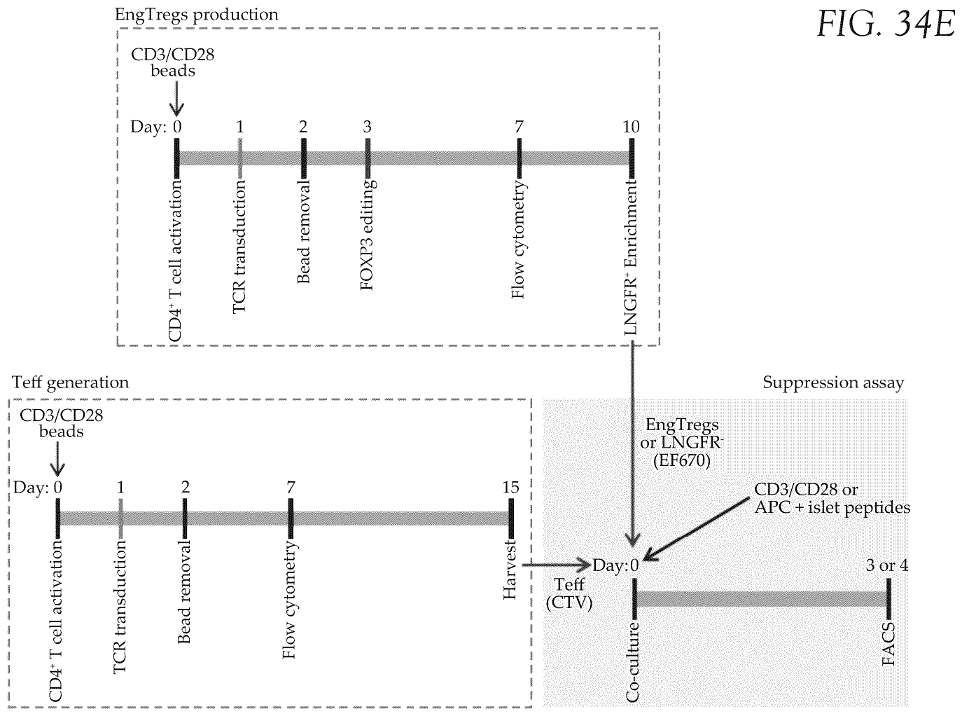

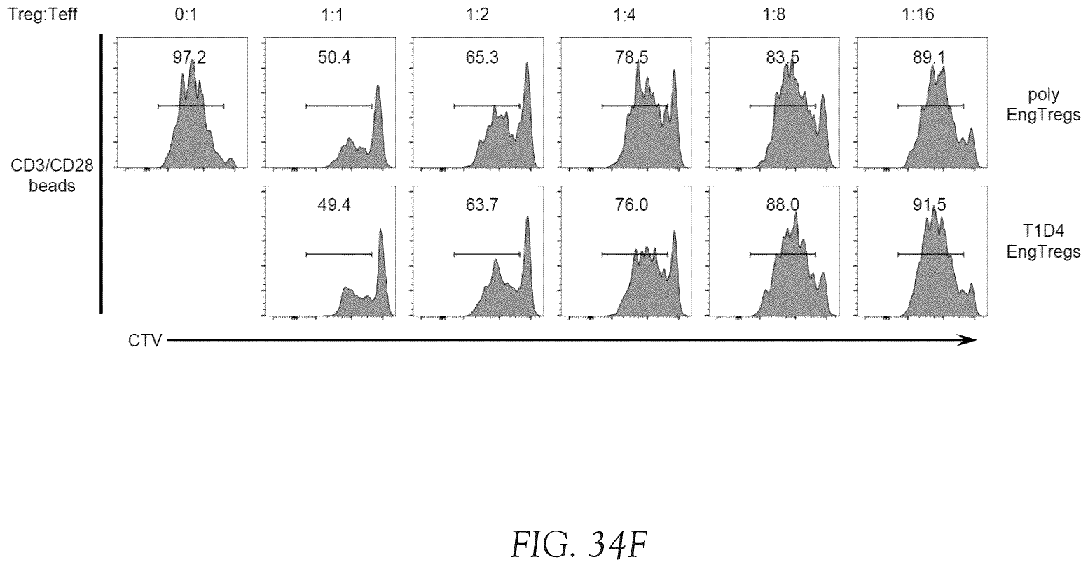

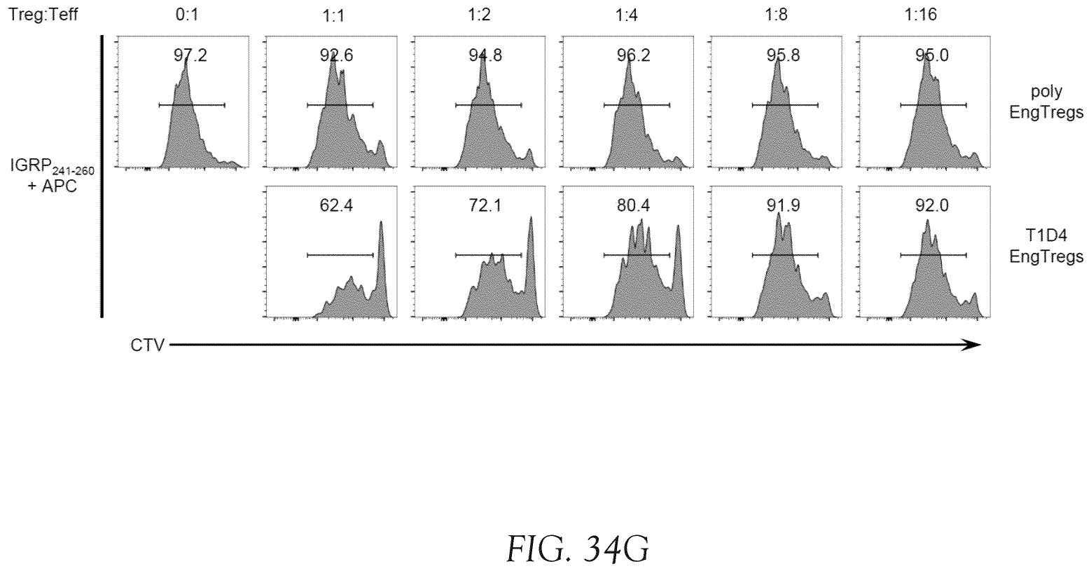

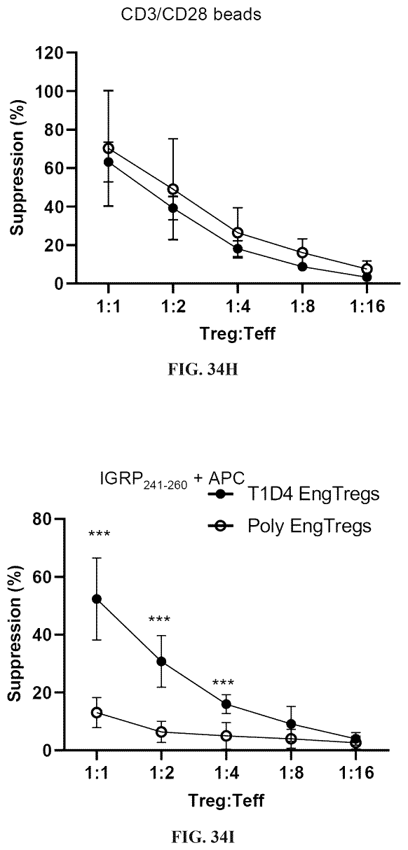

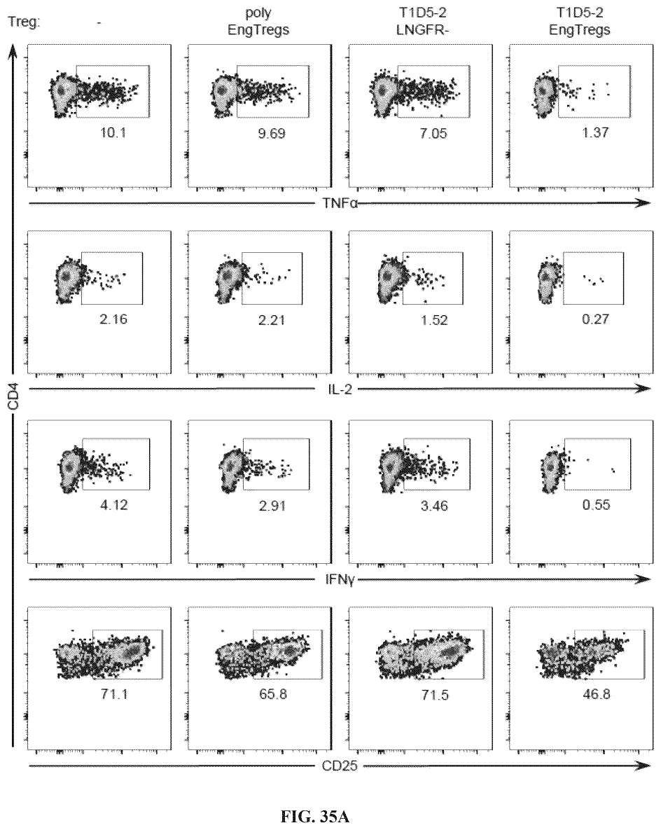

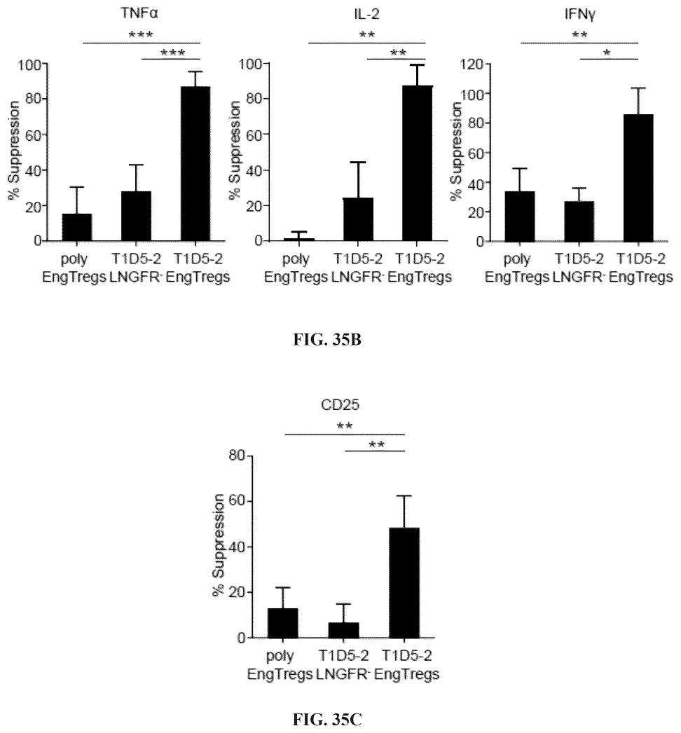

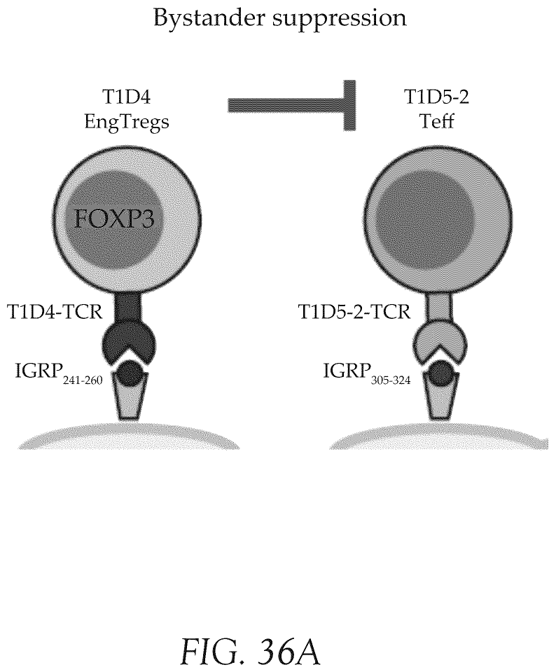

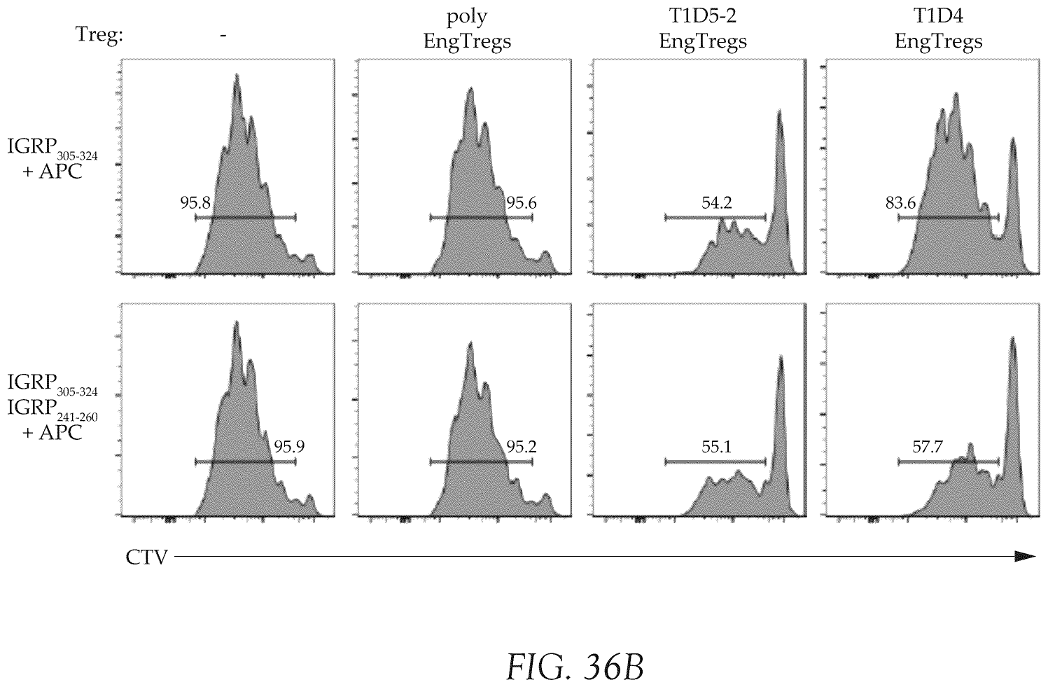

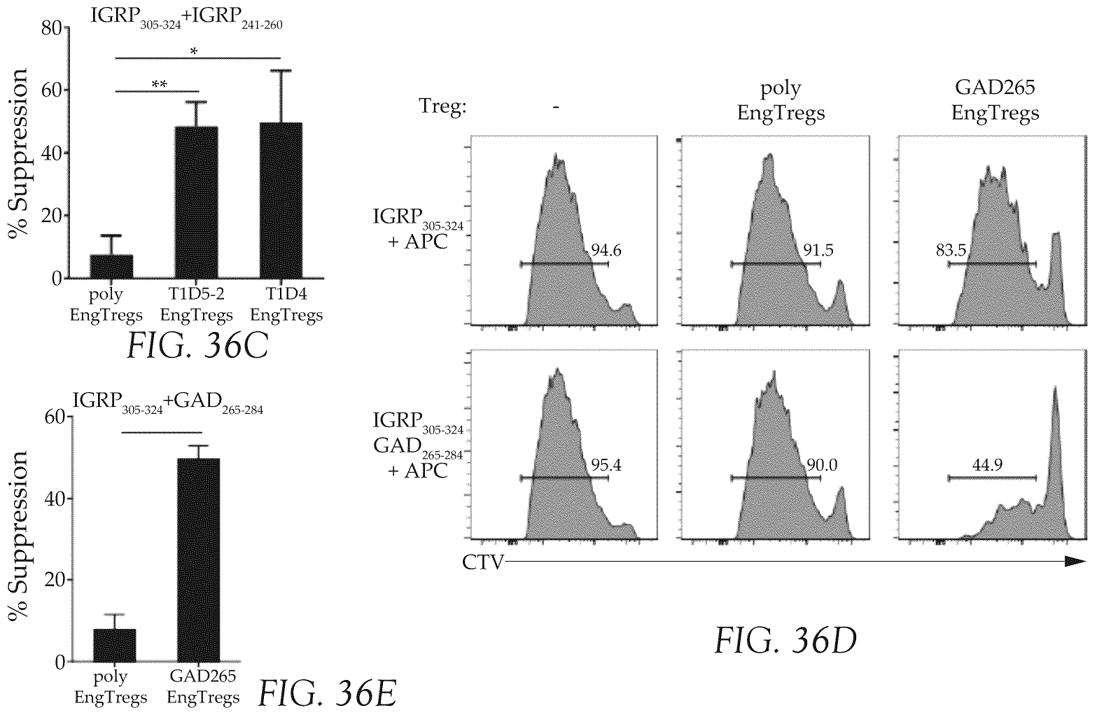

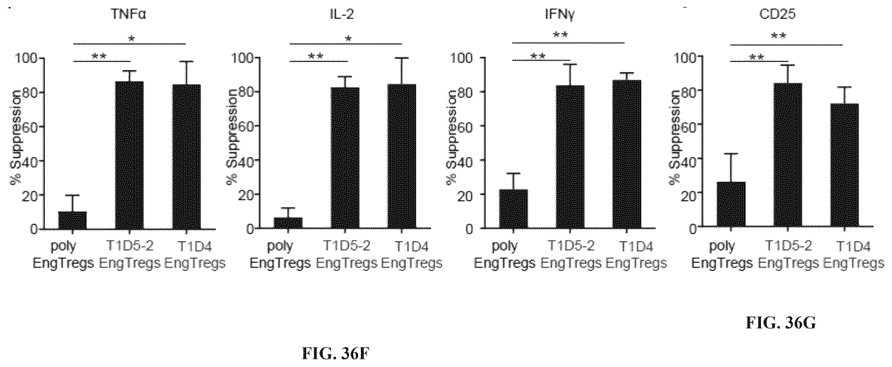

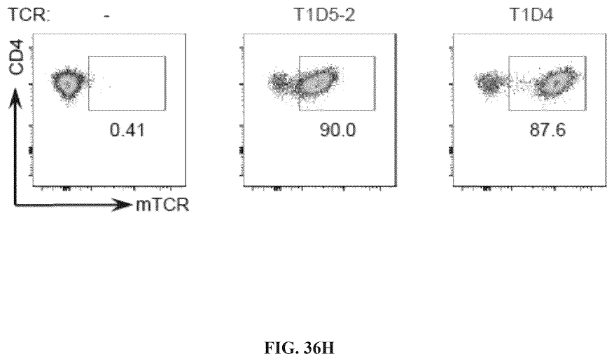

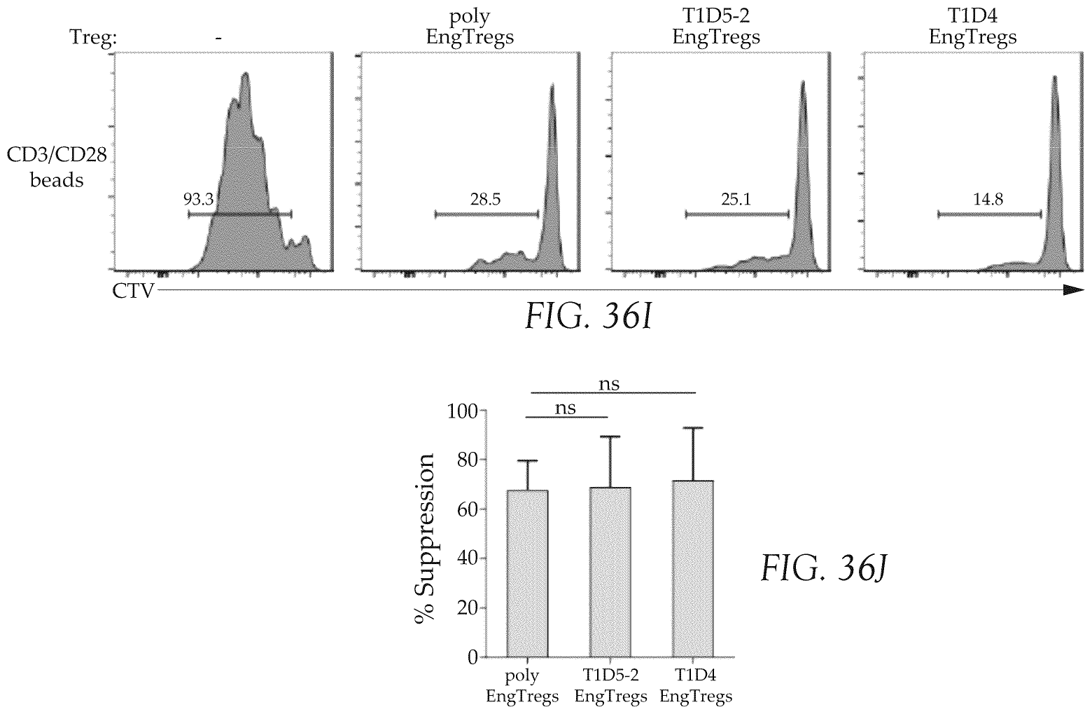

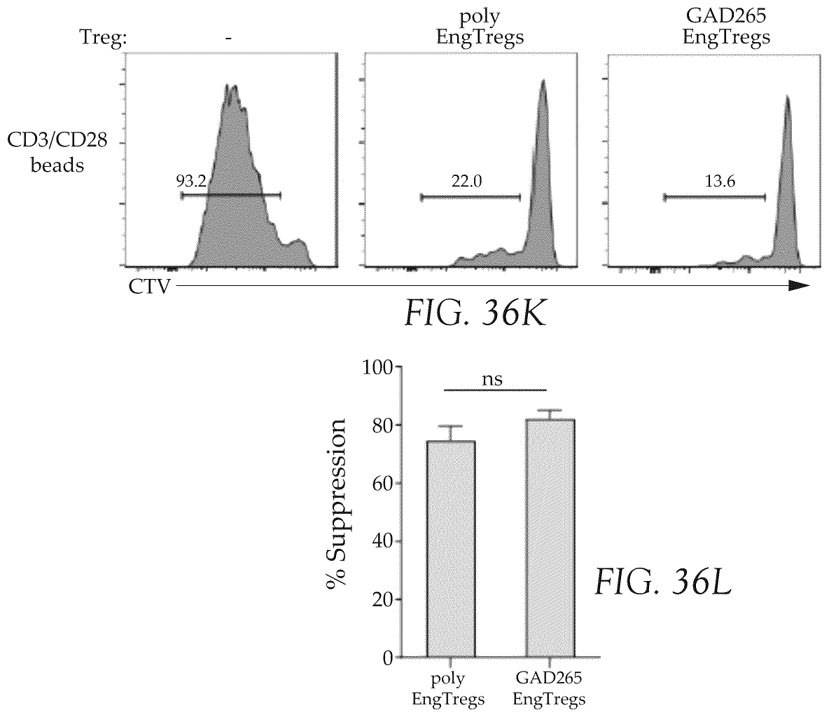

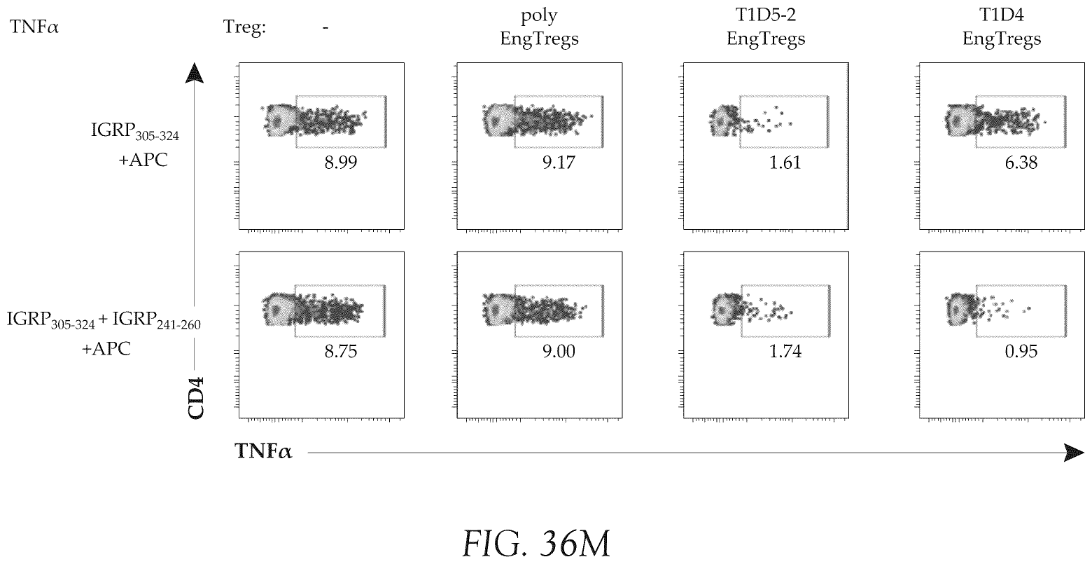

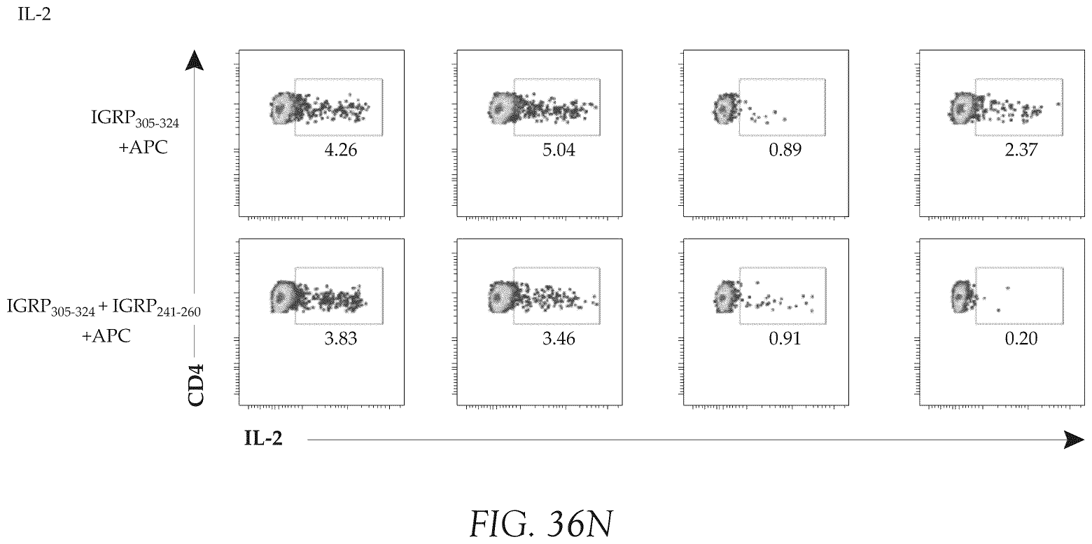

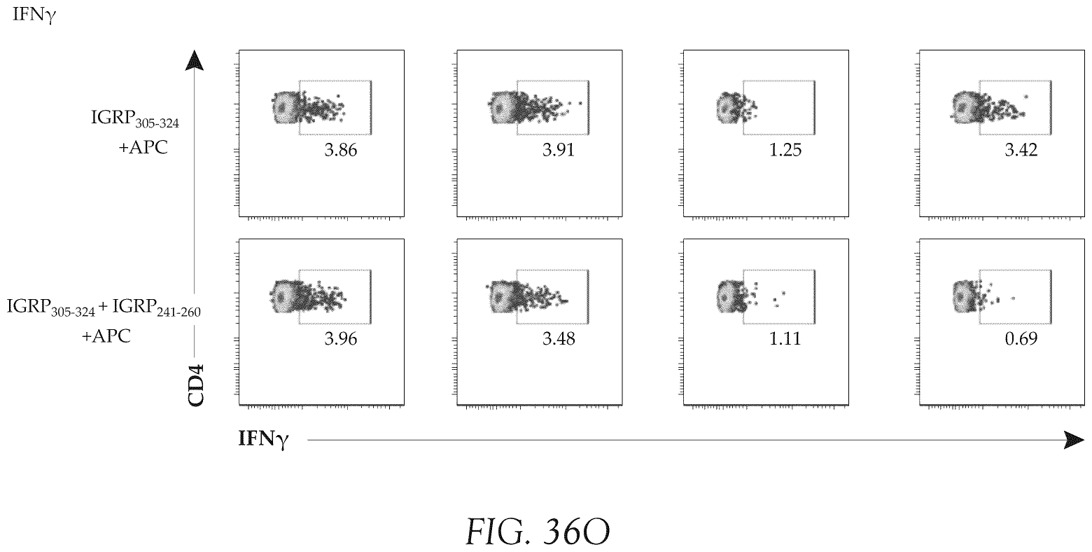

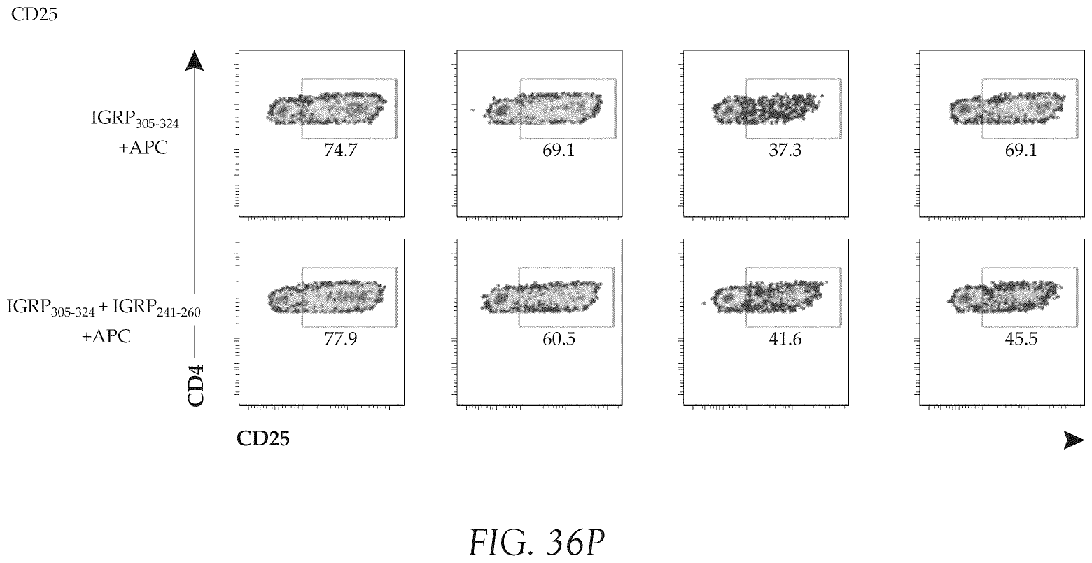

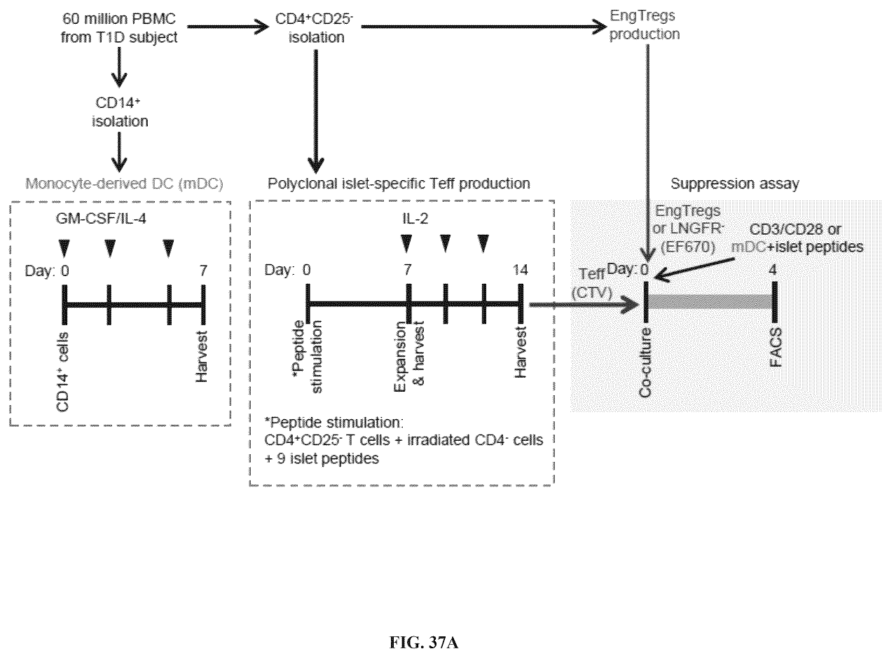

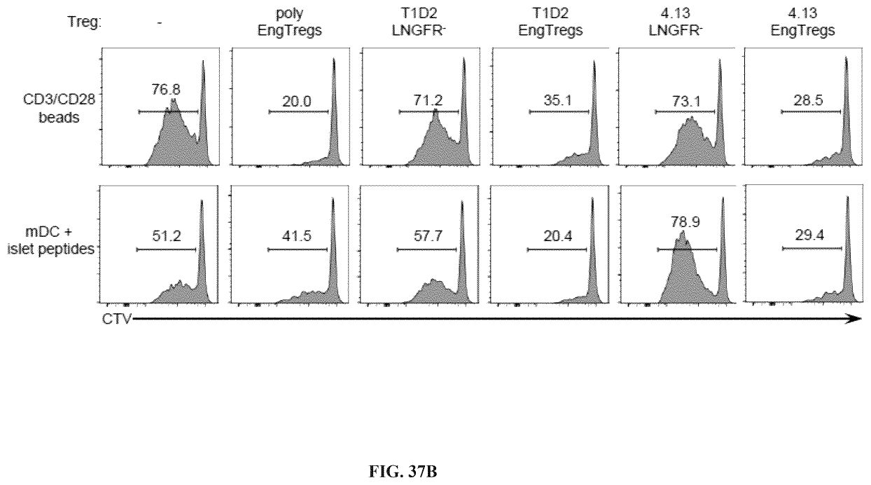

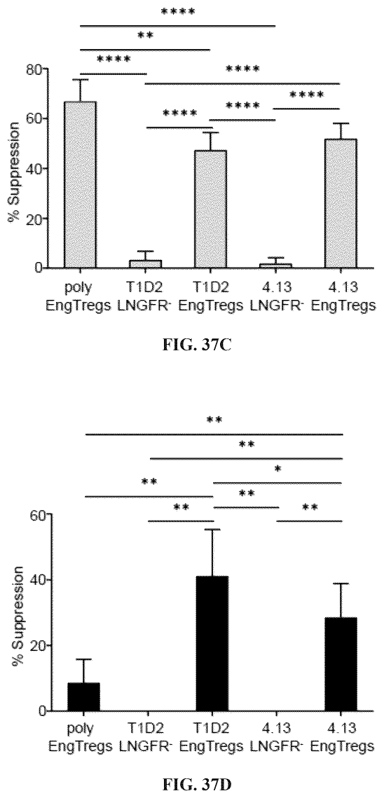

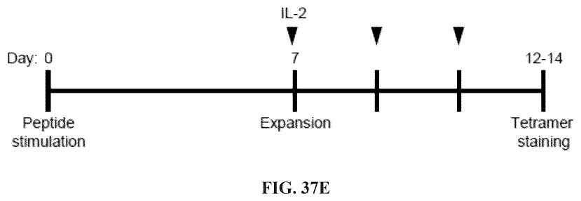

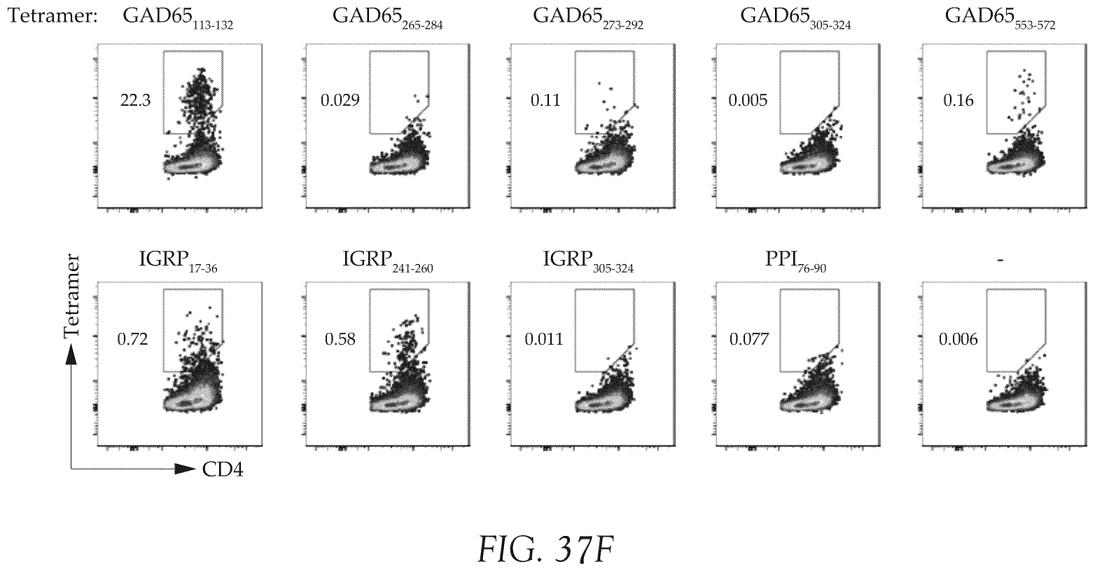

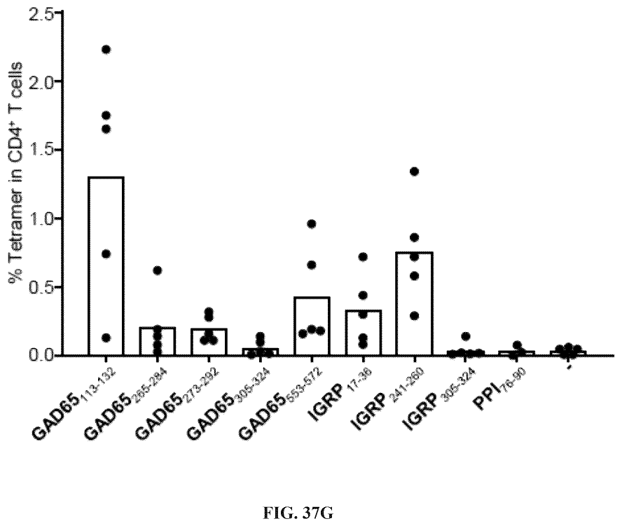

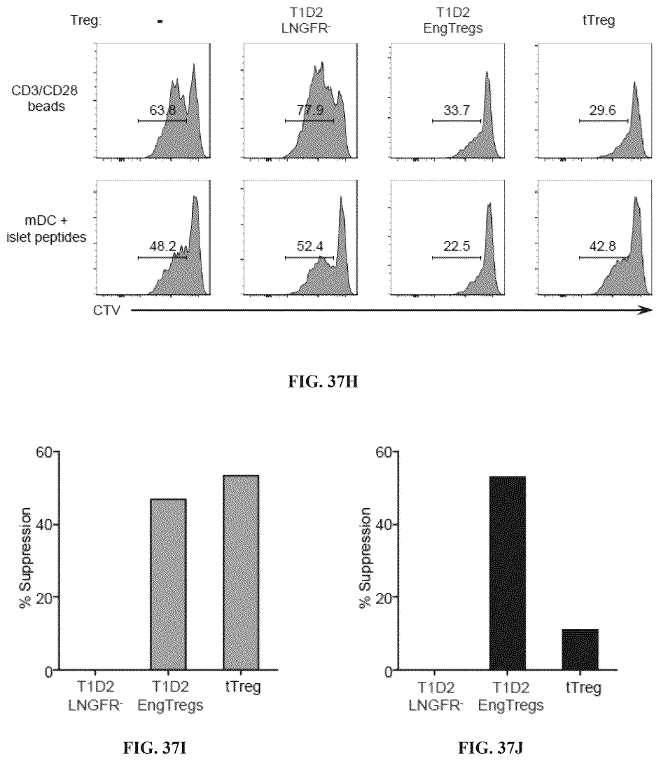

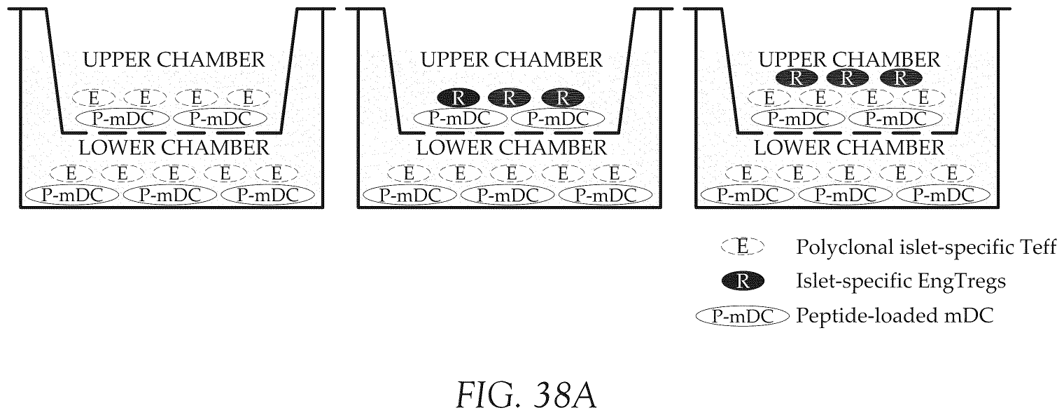

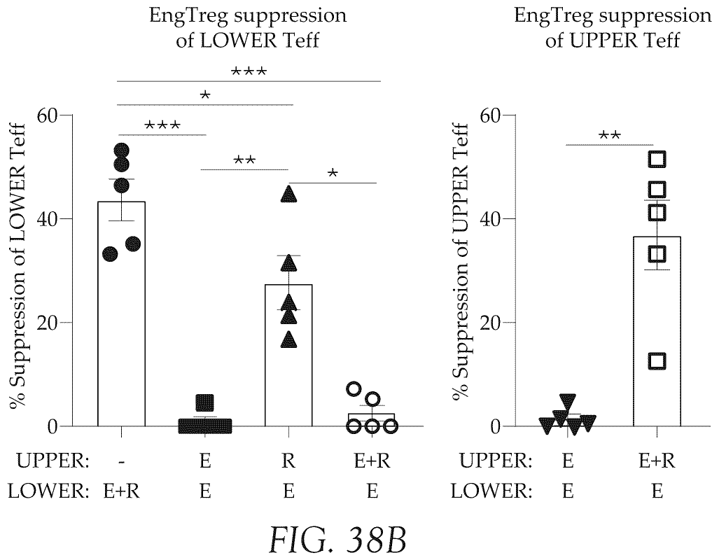

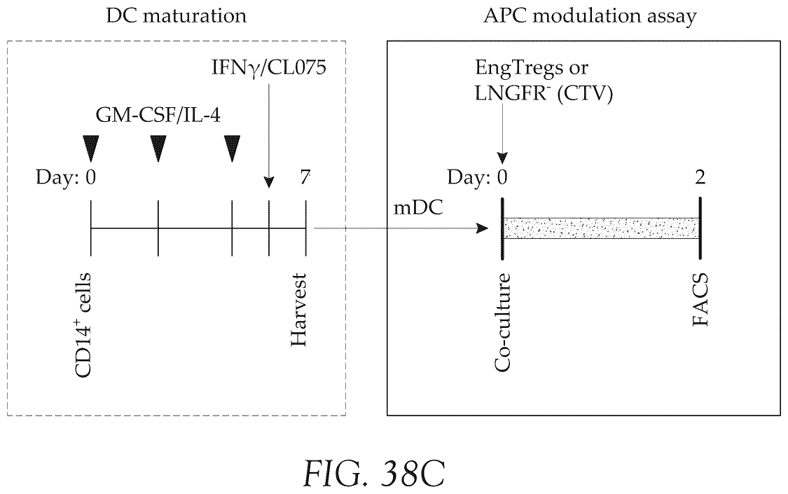

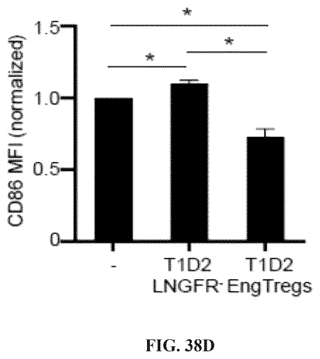

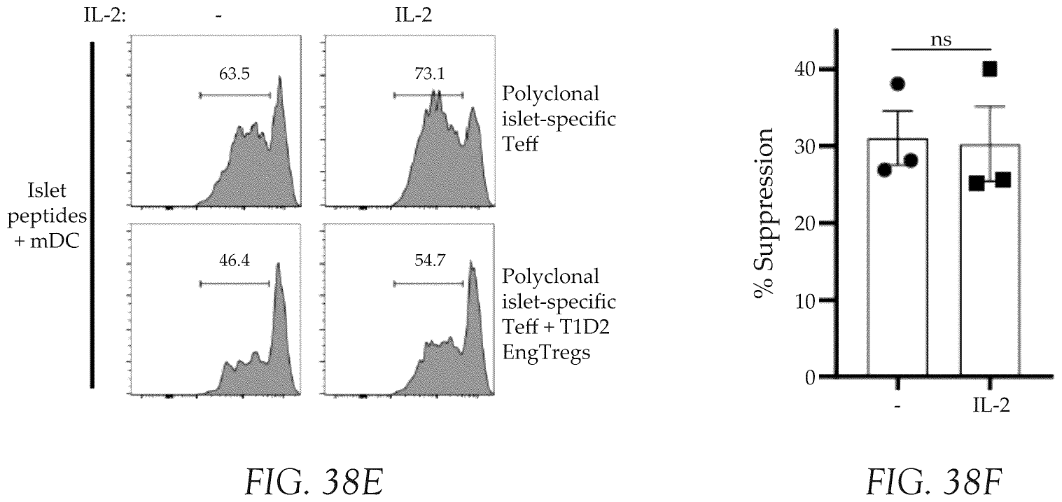

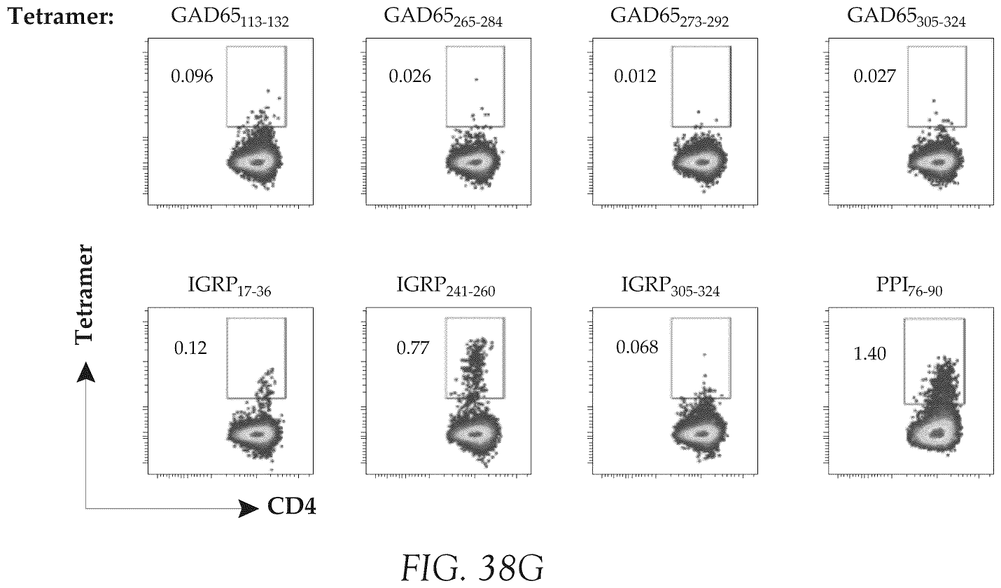

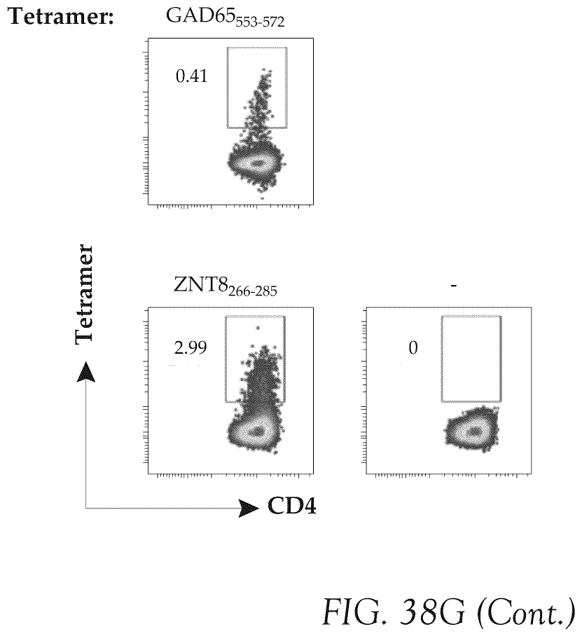

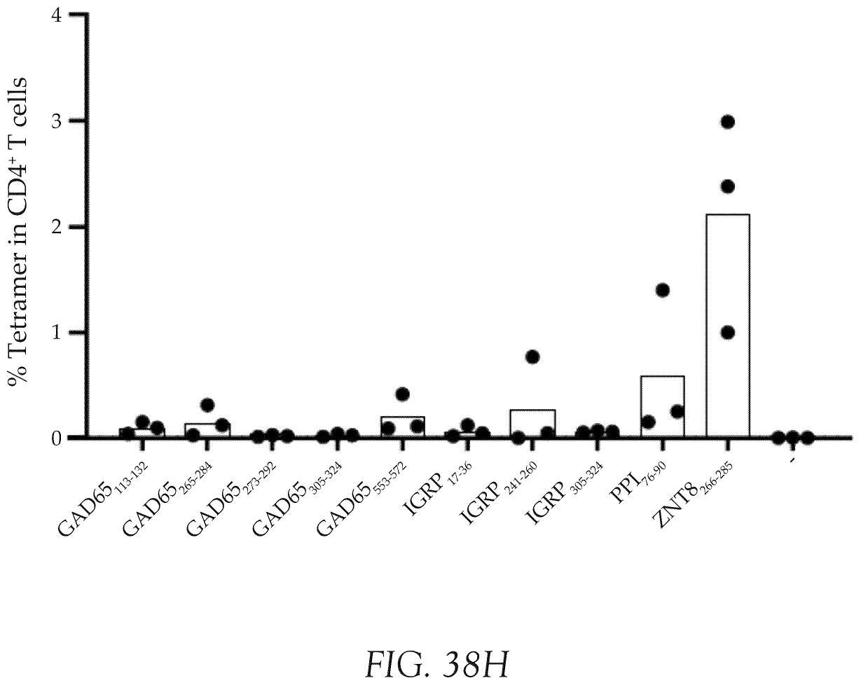

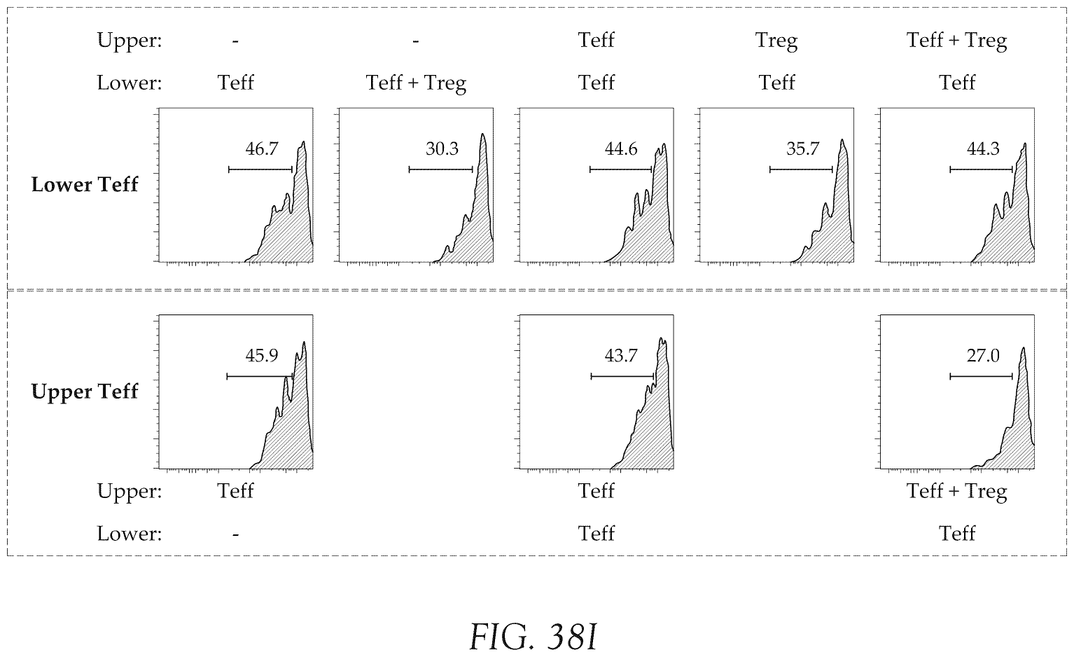

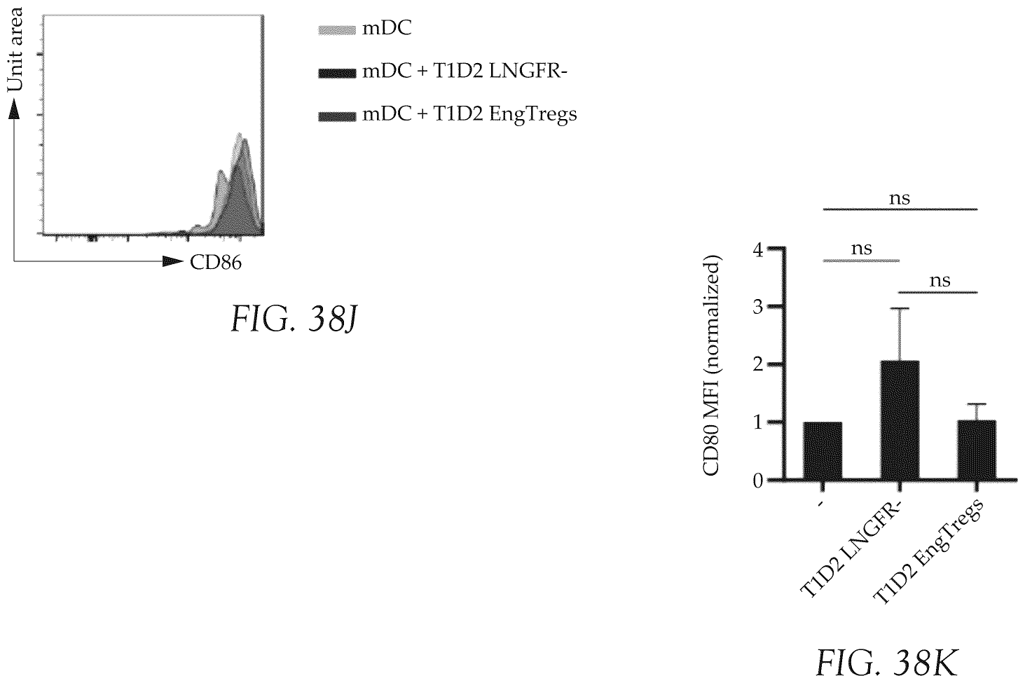

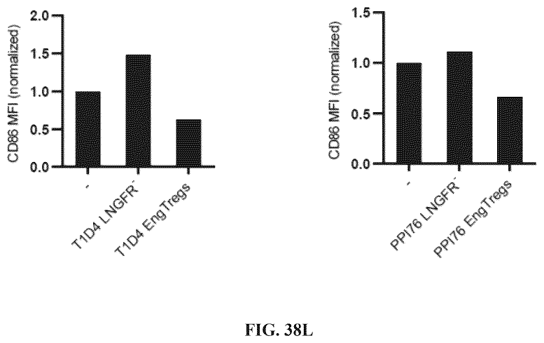

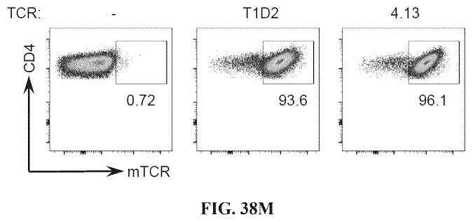

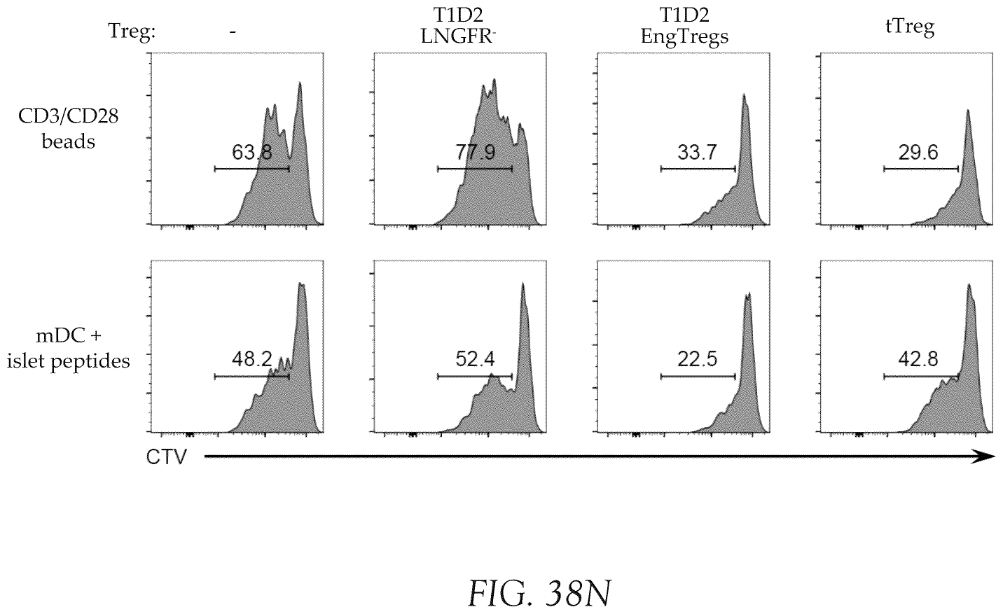

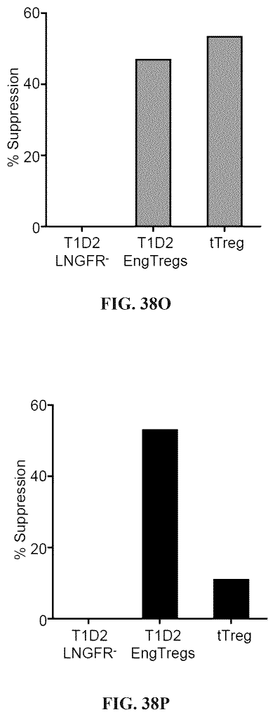

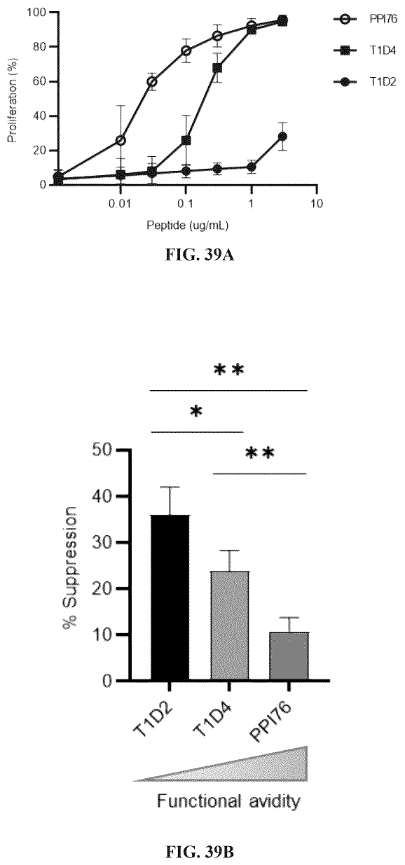

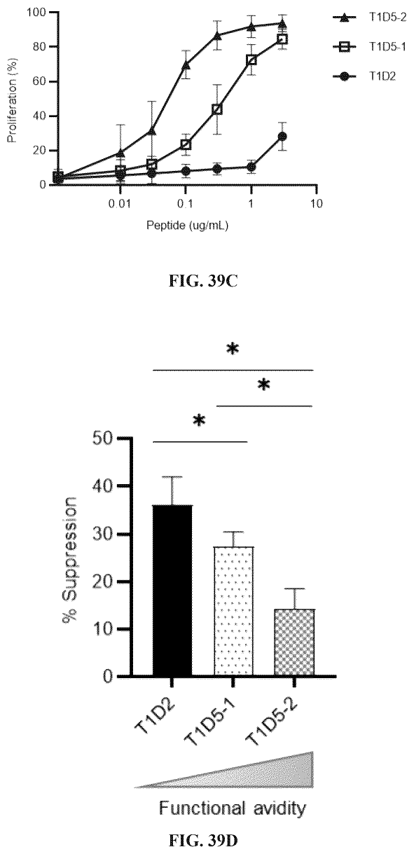

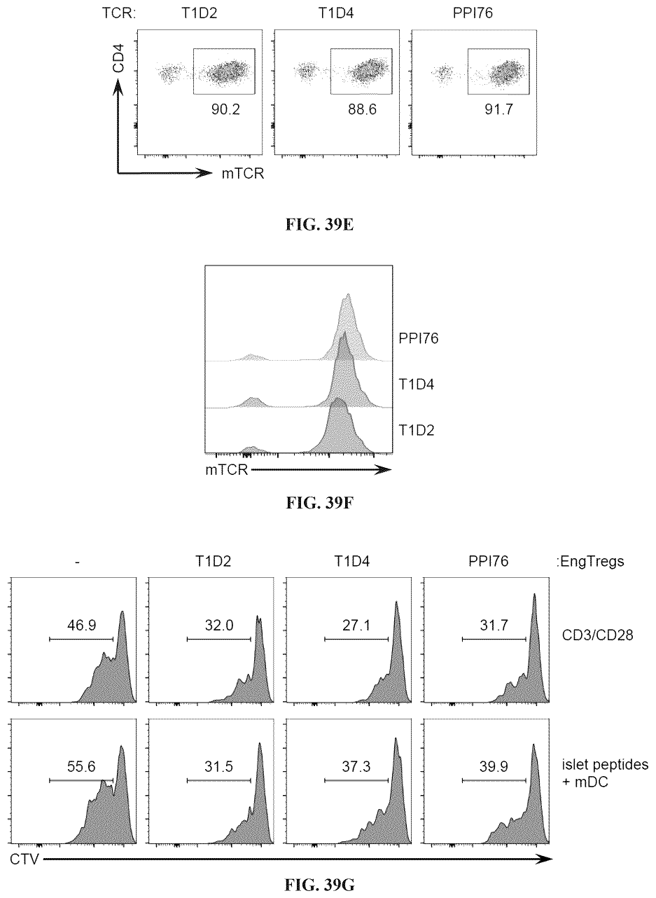

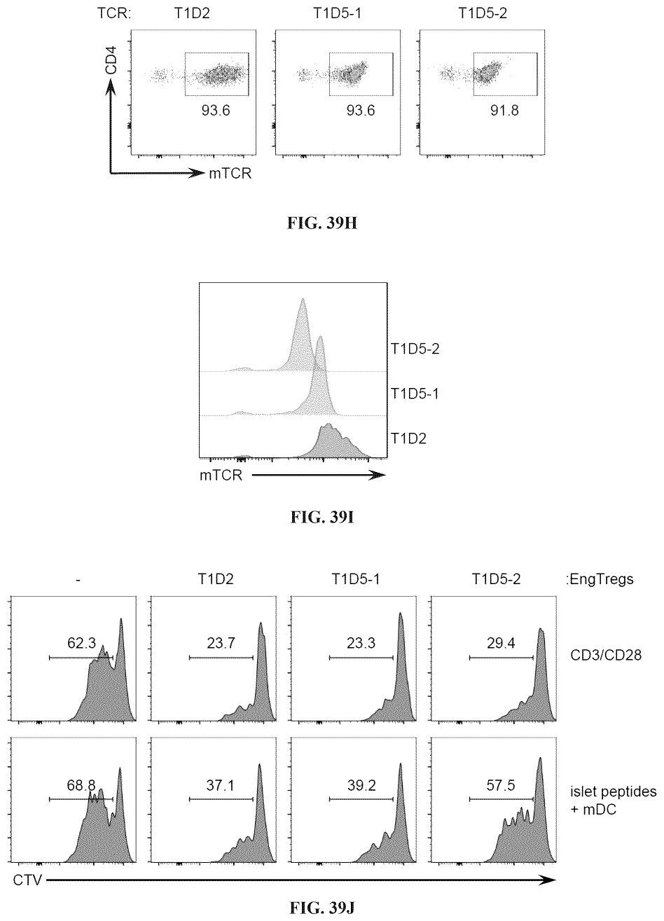

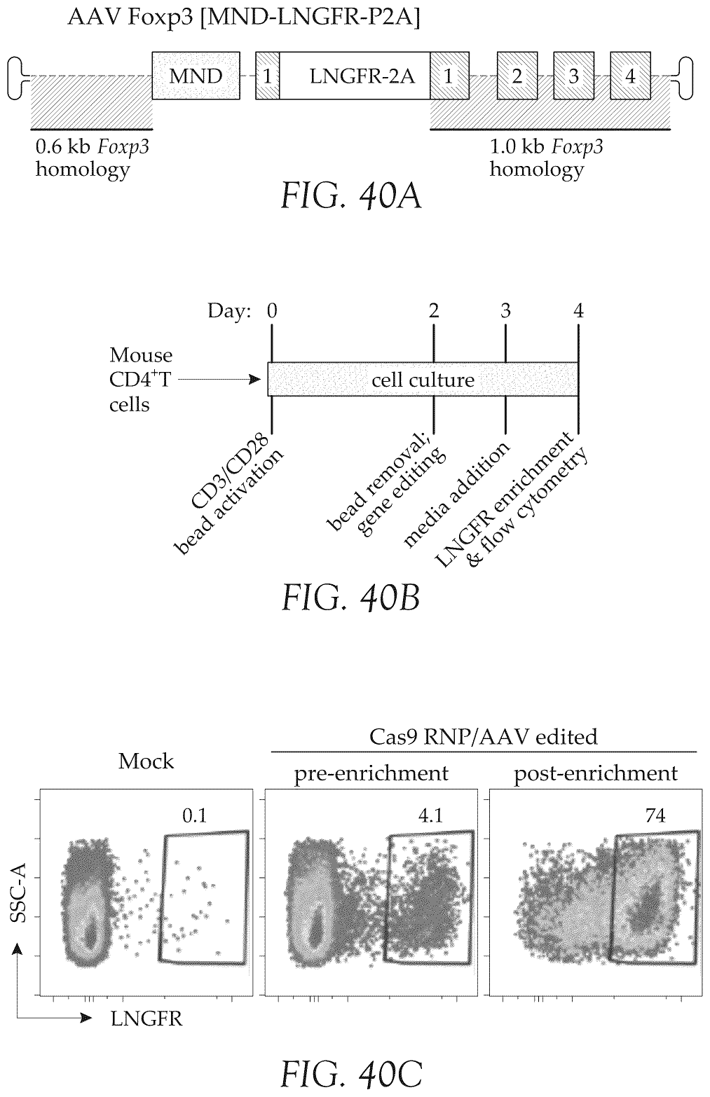

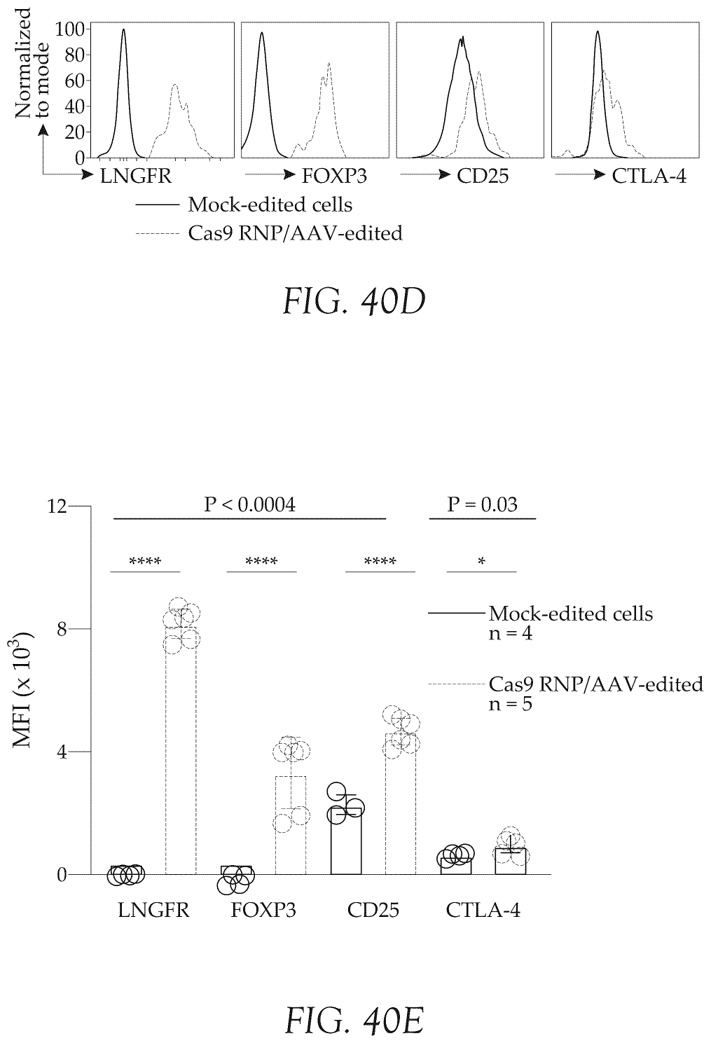

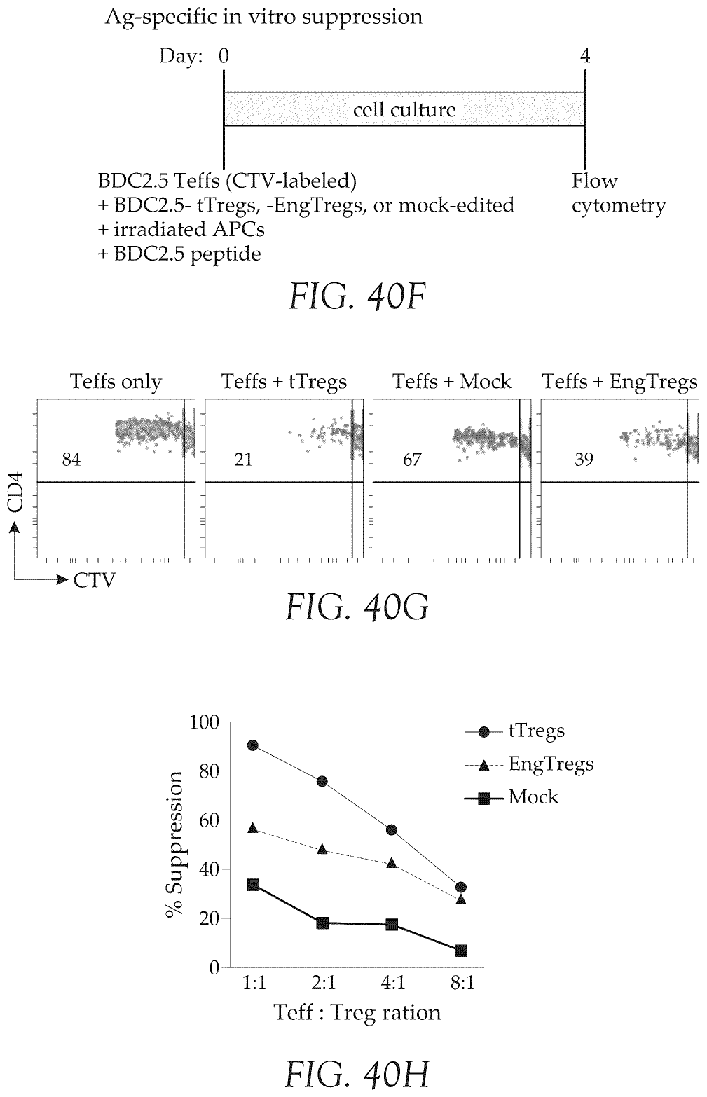

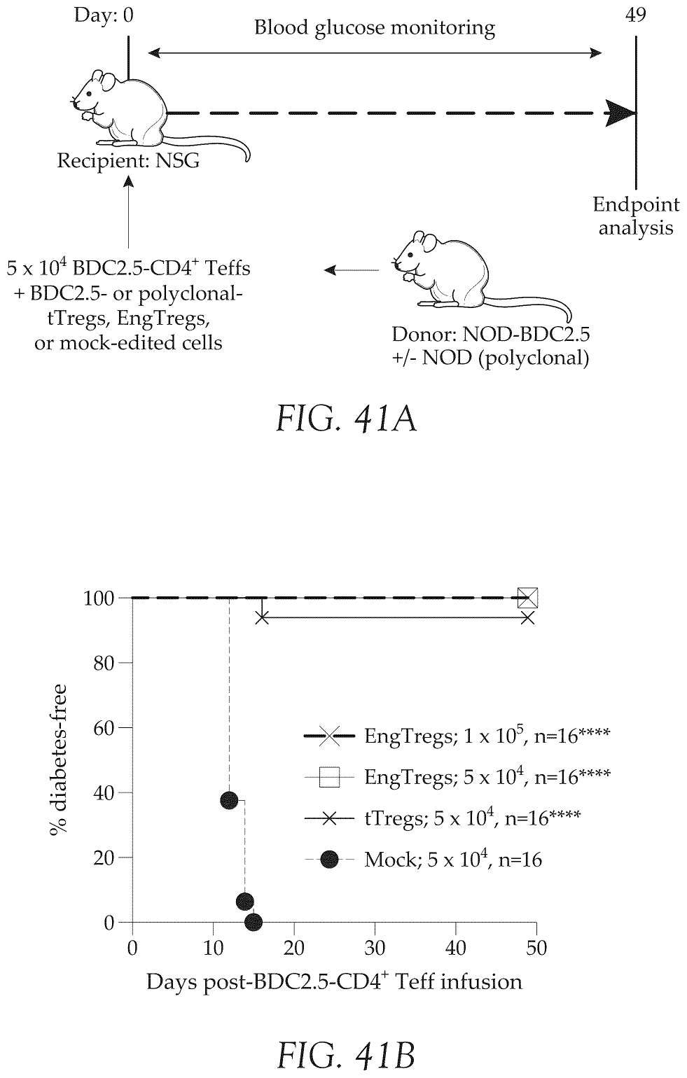

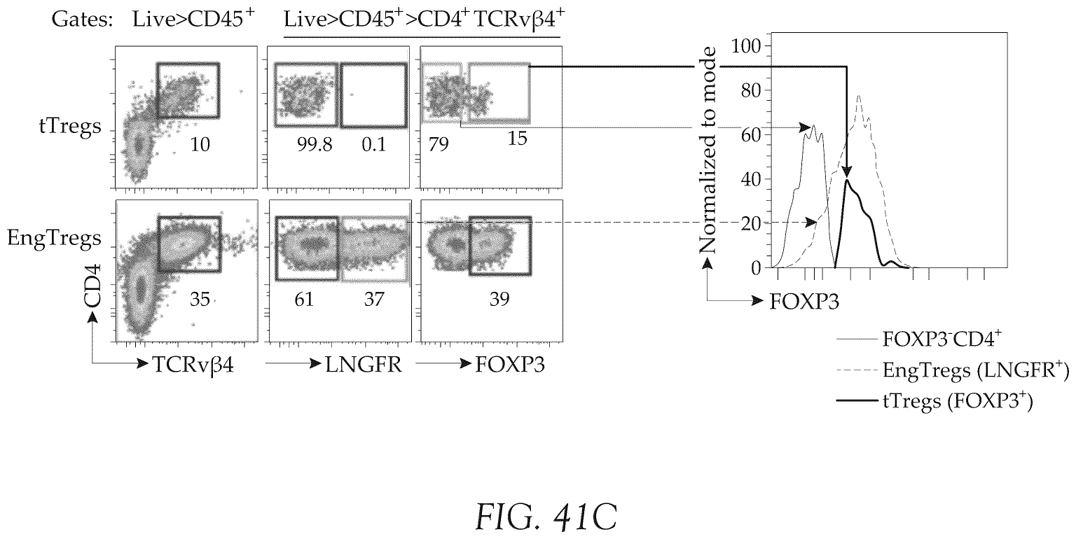

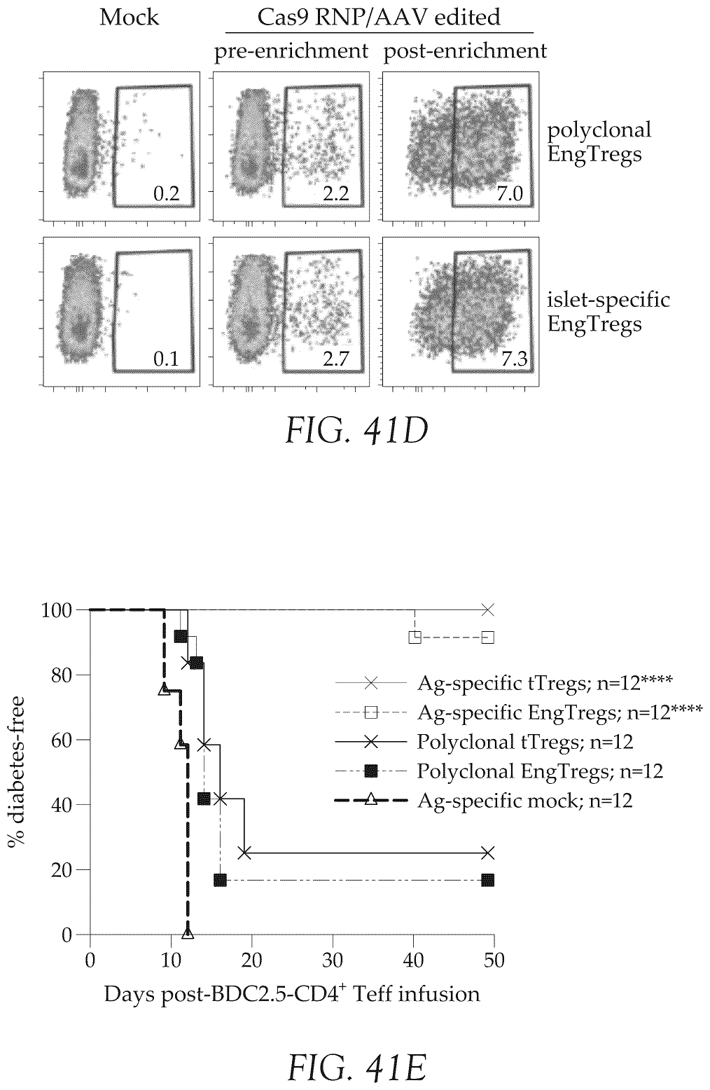

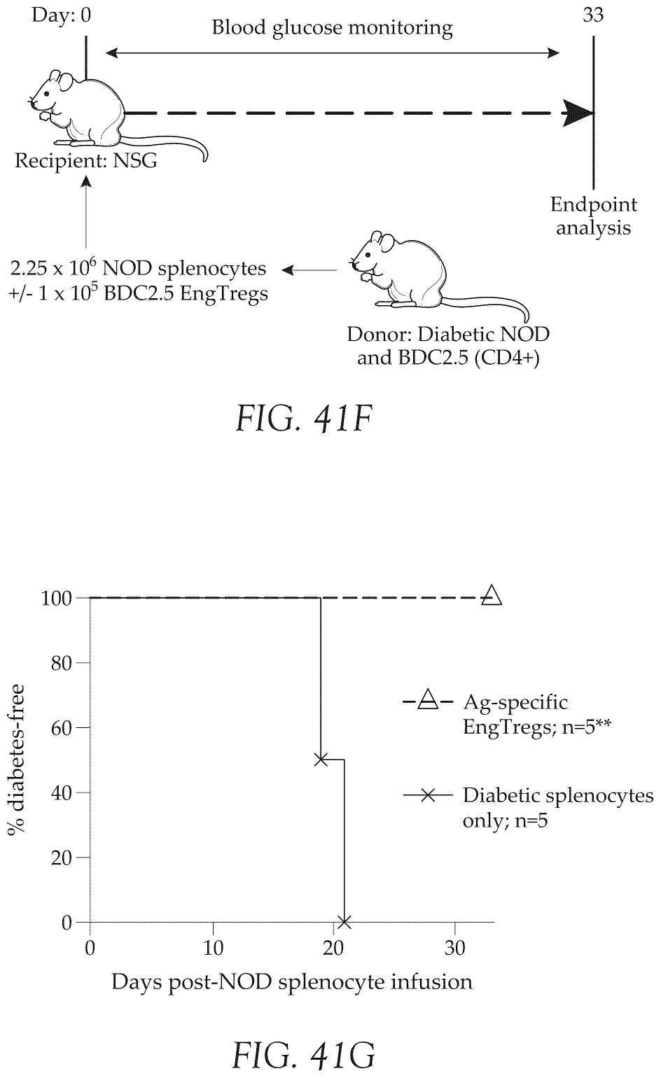

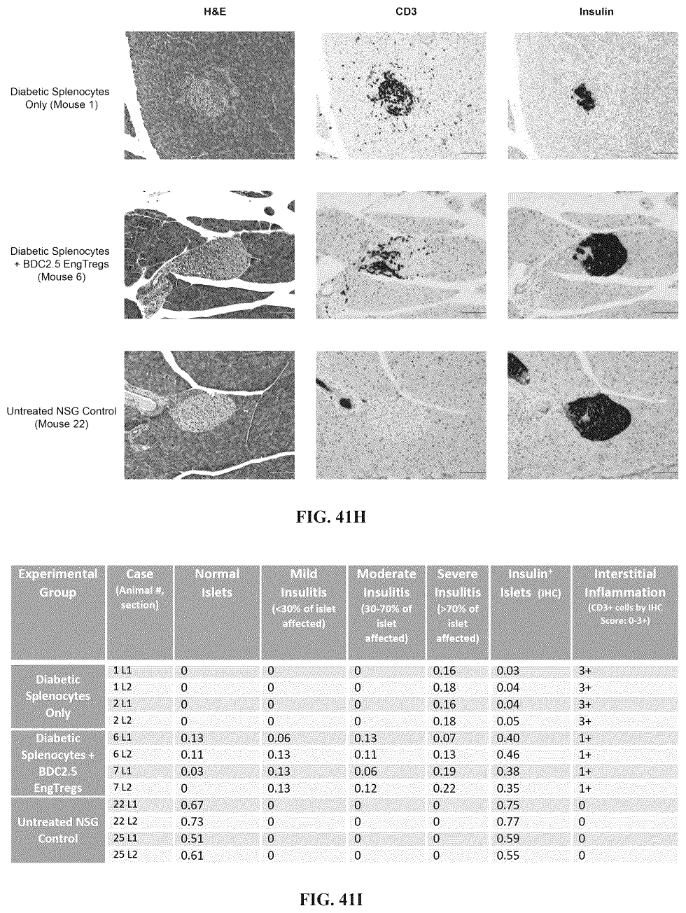

FIG. 7A and FIG. 7B compare suppressive functions of dual-edited EngTregs edited by targeted TRAC and FOXP3 locus editing, and Tregs generated by insertion of T1D2 or T1D5-1 TCR coding sequences by lentiviral vectors. FIG. 7A shows data for a Teff:DC ratio of 30:1. Using the assay described in FIG. 6C, the ability of dual-HDR edited hT1D2+/FoxP3+ and hT1D5-1+/FoxP3+ EngTregs to mediate bystander suppression. Proliferation of islet-antigen-specific Teff cells stimulated with a pool of islet peptides (containing 3 peptides of IGRP, 5 peptides of GAD65, 1 peptide of PPI, and 1 peptide of ZNT8) was measured in the presence and absence of hT1D2+/FoxP3+ and hT1D5-1+/FoxP3+ EngTregs, with Teff:Treg ratio of 1:1 and 1:0.5 (Treg1/2). Both hT1D2+/FoxP3+ and hT1D5-1+/FoxP3+ dual-edited EngTregs exhibited robust bystander suppression activity. In this study, hT1D2 dual-edited cells exhibited slightly more suppressive effect than hT1D5-1 dual-edited cells. The cells labeled T1D2 and T1D5-1 (not hT1D2 or hT1D5-1) represent the LV-edited Tregs described in FIG. 6C, which exhibited less suppression than dual-edited EngTregs expressing the human counterpart TCR (e.g., T1D2 v. hT1D2). FIG. 7B shows data for varying ratios of TeffDC. “T1D2” and “T1D5” denote Tregs engineered using lentiviral vectors encoding murine TCRs without endogenous TCR knockout. The data demonstrates reproducible polyclonal bystander suppression with TeffDC ratios of 5:1, 10:1, 20:1 and 30:1. The dual-edited hT1D2+/FoxP3+ and hT1D5-1+/FoxP3+ EngTregs had superior suppressive activity compared to LV-edited Tregs expressing murine TCRs having the same specificity (e.g., hT1D5-1 v. T1D5-1).

FIG. 8A-8C show phenotype of Tregs engineered using dual-editing. Antibodies were used to detect expression of human T1D2 (anti-TCRVβ 13.6) and human T1D5-1 (anti-TCRVβ 7.2). FIG. 8A shows expression of TCRBb proteins. FIG. 8B shows expression of TCR, FoxP3, and CD25. TCRVβ 13.6 staining was observed in both hT1D2/FoxP3 targeted dual-edited and T1D2 LV-edited cells. TCRVβ 7.2 staining was observed in both hT1D5-1/FoxP3 targeted dual-edited and T1D5-1 LV-edited cells. Higher signal was observed in cells expressing T1D2 and hT1D2 TCRs, compared to those expressing T1D5-1 or hT1D5-1 TCRs, respectively, which may be due to variation in either TCR expression or staining effectiveness by anti-TCRVβ 7.2 antibody. Both hT1D2+/FoxP3+ and hT1D5-1+/FoxP3+ dual-edited EngTreg cells exhibit a Treg phenotype as measured by FoxP3 and CD25 expression (upper and lower right figures). Notably, both hT1D2+/FoxP3+ and hT1D5-1+/FoxP3+ dual-edited EngTreg cells exhibited higher levels of CD25, relative to LV-edited cells expressing the counterpart murine TCR (e.g., hT1D2 Dual v. T1D2). FIG. 8C shows that hT1D2+/FoxP3+ and hT1D5-1+/FoxP3+ dual-edited EngTreg cells frequently expressed CD39 and CD73, two surface proteins associated with the generation of adenosine and implicated in the suppression of Teff by Tregs and of HLA-DR. Dual-edited T1D2 and T1D5-1 EngTreg cells expressed all three Treg markers evaluated (CD39, CD73, and HLA-DR), at higher frequencies compared to LV-edited Treg cells expressing the counterpart murine TCR (e.g., hT1D2 Dual v. T1D2).

FIG. 9 depicts a graph of initial levels of dual-editing to prepare T1D4 EngTreg cells.

FIG. 10 depicts enrichment of dual-edited cells with rapamycin for T1D4 EngTreg cells.

FIG. 11 depicts a graph of levels of editing rates in T1D4 EngTreg cells pre- and post-enrichment using rapamycin.

FIG. 12 depicts a graph of levels of FoxP3, CD25 and CTLA-4 in T1D4 EngTreg cells in comparison to mock edited cells.

FIG. 13 depicts a graph of relative levels of TNF-α, IFN-γ, and IL-2 production in T1D4 EngTreg cells in comparison to mock edited cells.

FIG. 14 depicts a graph of relative expression of TGF-β in T1D4 EngTreg cells in comparison to mock edited cells.

FIG. 15 depicts graphs of relative suppression of T1D4 Teff cells by T1D4 EngTreg cells or mock edited cells stimulated by anti-CD3/CD28 stimulation, or APC+IGRP₂₄₁₋₂₆₀ stimulation.

FIG. 16 depicts graphs for secretion of TNF-α, IFN-γ, and IL-2 in T1D4 Teff cells cocultured with either T1D4 EngTreg or mock edited cells.

FIG. 17 depicts of relative suppression of PPI specific Teff cocultured with either T1D4 EngTreg or mock edited cells stimulated using antigen presenting cells with PPI peptide alone, or both PPI peptide and IGRP peptides.

FIG. 18 depicts PPI specific Teff cytokine secretion when cultured with T1D4 EngTreg or mock edited cells and APC with PPI₇₆₋₉₀ peptide, or with PPI₇₆₋₉₀ peptide and IGRP₂₄₁₋₂₆₀.

FIGS. 19A-19C show an overview of Type 1 diabetes and the function of GNTI-122, an engineered T regulatory cell therapy for the treat of Type 1 diabetes. FIG. 19A shows a mechanism of Type 1 diabetes pathogenesis, specifically the T-lymphocyte-mediated killing of insulin-producing beta cells. FIG. 19B shows suppression of T effector cells, and consequent protection of pancreatic islet cells, by GNTI-122 engineered Treg cells. FIG. 19C shows a schematic of the development process of GNTI-122.

FIG. 20 shows the manufacturing process of GNTI-122 engineered Tregs from autologous cells.

FIG. 21 shows selective expansion of GNTI-122 cells during the manufacturing process. The frequency of GNTI-122 cells is measured by flow cytometry. FACS analysis of GNTI-122 cells and mock-engineered cells is shown 3 days after editing (left) and at the time of cryopreservation (right).

FIGS. 22A-22E show the effects of rapamycin stimulation on GNTI-122 Treg cells and mock-engineered cells. FIG. 22A depicts the effects of rapamycin administration on the in vivo engraftment of GNTI-122 Treg cells. FIG. 22B depicts phosphorylated STAT5 (pSTAT5) median fluorescence intensity (MFI) in GNTI-122 or mock-engineered cells in response to varying doses to rapamycin in culture. Repeated measures ANOVA cell type, dose and interaction, p<0.0001, Sidak's multiple comparison tests at each dose (*p<0.05,***p<0.001). The errors bars represent mean+/−SEM, N=3 donors. The cells are gated on live CD3+CD4+ population of both mock-engineered and GNTI-122 cells. FIG. 22C shows cell survival in culture (measured by fold expansion) in the presence of 10 mM rapamycin without TCR stimulation. FIG. 22D shows cell survival in culture (measured by fold expansion) in the presence of 10 mM rapamycin with TCR stimulation via anti-CD3/CD28 beads. FIG. 22E shows fold expansion with TCR stimulation in the presence of rapamycin at concentrations ranging from 0 to 30 nM. 2-way ANOVA with Tukey's multiple comparison test, significance displayed for paired conditions at day 8 (*p<0.05, **p<0.01, ***p<0.001, ****p<0.0001).

FIGS. 23A-23H show expression of Treg-associated markers and suppression of T effector (Teff) cells by GNTI-122 and mock-engineered cells. GNTI-122 cells and their corresponding mock controls generated in parallel were stained after thawing and a 3-day rest in culture. Mock-edited cells were gated on CD4+ cells, and GNTI-122 cells were gated on islet-specific T cell receptor (isTCR)FoxP3+ cells. Representative data in each of FIG. 23A and FIG. 23B are shown for one donor, with phenotype reproduced in cells produced independently from 6 distinct donors. FIG. 23C shows direct suppression of Teff cells expressing the same TCR as GNTI-122. FIG. 23D shows bystander suppression of Teff cells expressing a different TCR specific to a different T1D-associated antigen, preproinsulin (PPI). FIG. 23E shows suppression of a polyclonal Teff cell population expressing TCRs specific to any of 9 different cognate peptides of T1D-associated antigens. FIG. 23F shows editing efficiency in EngTregs generated from subjects with T1D. FIG. 23G shows enrichment efficiency in EngTregs generated from subjects with T1D. FIG. 23H shows phenotyping of EngTregs generated from subjects with T1D.

FIGS. 24A-24B show the in vitro properties of GNTI-122 cells. FIG. 24A shows cytokine production and Treg activation marker expression by mock-engineered cells, GNTI-122 cells alone, and GNTI-122 cells contacted with rapamycin, following stimulation with PMA/ionomycin/monensin or with anti-CD3/CD28 beads. The relative MFI levels were normalized to mock cells. *** or **** indicates statistically significant difference by 2-way ANOVA. Representative donor data shown, reproduced across 6 independent donors. FIG. 24B shows suppression of Teff cells expressing the same isTCR by mock-engineered cells or GNTI-122 cells. Mock-engineered or GNTI-122 cells were cultured with autologous isTCR⁺FoxP3⁻ Teff cells, and stimulated with monocyte-derived dendritic cells loaded with cognate peptide recognized by the isTCR. Suppression indicates inhibition of Teff as determined by flow cytometry analysis of Teff activation. *** or **** indicates a statistically significant difference by 2-way ANOVA. Representative donor data shown, reproduced across 3 independent donors.

FIGS. 25A-25C show the experimental design and efficacy of mouse engineered Treg therapy in an adoptive transfer Type 1 diabetes model. FIG. 25A depicts the experimental timeline. FIGS. 25B-25C shows diabetes-free survival (FIG. 25B) and blood glucose (FIG. 25C) in recipient NOD.Cg-Prkdc^{scid} Il2rg^{tm1Wjl}/SzJ (NSG™) mice, after intravenous injection of splenocytes from diabetic non-obese diabetic (NOD) mice (T1D splenocytes), followed by intravenous injection of BDC2.5 mouse engineered regulatory T cells (mEngTregs), either 7 or 15 days after T1D splenocyte administration.

FIGS. 26A-26B show localization of mEngTregs and suppressive function in vivo. Mice were administered T1D splenocytes on day 0, followed by mEngTregs or no treatment on day 14 post-T1D splenocyte administration, and euthanized on day 22 to quantify mEngTreg and CD8+ Teff memory cells in blood, bone marrow, liver, pancreas, and spleen. FIG. 26A depicts quantification of mEngTregs (isTCR⁺FoxP3⁺). FIG. 26B shows the quantification of CD8⁺ T effector memory (CD44⁺CD62L⁻) cells.

FIGS. 27A-27C show reduction of pancreatic islet inflammation and preservation of beta cells. The mice of FIGS. 25A-25C were euthanized at 43 days post-T1D splenocyte administration, for histological analysis of pancreata. FIG. 27A shows severity scores for pancreatic islet inflammation quantified via hematoxylin and eosin (H&E) staining.

FIG. 27B shows the quantification of beta cell mass by insulin staining of pancreata. Approximately 20 pancreatic islets were quantified per mouse. FIG. 27C shows representative H&E staining and insulin staining of pancreata from mice administered T1D splenocytes, and optionally mEngTregs, at day 43 post T1D splenocyte administration.

FIG. 28 shows a mouse study was conducted where mEngTregs were administered 7 days after the diabetogenic splenocytes.

FIGS. 29A-29E show editing of CD4+ T cells to express one of a panel of TCRs, and phenotypic characterization of edited cells. FIG. 29A shows an overview of editing, stimulation, and analysis. FIG. 29B shows a representative gating strategy for evaluating expression of surface markers CD69, CD137, and CD154 post-stimulation (day 8). FIG. 29C shows expression of surface markers CD69, CD137, and CD154 after 20 hours of stimulation with HLA-DR-expressing K562 cells pulsed with cognate IGRP 305-324 or IGRP 241-260 peptide. FIG. 29D shows a representative gating strategy for evaluating TNF-α and IFN-γ production post-stimulation (day 14). FIG. 29E shows TNF-α and IFN-γ production after 5 hours of stimulation with HLA-DR-expressing K562 cells pulsed with cognate IGRP 305-324 or IGRP 241-260 peptide.

FIGS. 30A-30B show dose response of T1D TCR-expressing CD4+ T cells to stimulation with IGRP 305-324 peptide. Cells were cultured in the presence of HLA-DR4-expressing K562 cells for a 20 hours, and analyzed by flow cytometry. FIG. 30A shows dose response as measured by CD154 surface expression intensity. FIG. 30B shows dose response as measured by % CD137-expressing cells. Dashed lines=50% maximum response of each cell population (by donor).

FIGS. 31A-31D show tolerance of T1D2 to substitutions in IGRP 305-324 peptide. FIGS. 31A and 31B show activation of T1D2 TCR-expressing CD4+ T cells, as measured by CD154 expression intensity (FIG. 31A) or % CD137-expressing cells (FIG. 31B) in the presence of antigen-presenting cells pulsed with one of a panel of alanine-substituted peptides. T cells were cultured for 20 hours in the presence of HLA-DR4-expressing K562 cells that had been pulsed with IGRP 305-324 peptide, or one of a panel of peptides having an alanine substitution at different positions, and analyzed by flow cytometry. FIGS. 31C and 31D show activation of T1D2 TCR-expressing CD4+ T cells, as measured by CD154 expression intensity (FIG. 31C) or % CD137-expressing cells (FIG. 31D) in the presence of antigen-presenting cells pulsed with one of a panel of potential off-target peptides derived from pathogens of human relevance. “Control” indicates CD4+ T cells expressing ZNT266 TCR.

FIG. 32 provides an overview of study design for a Phase 1/2 study to evaluate GNTI-122 in adult and pediatric subjects recently diagnosed with T1D.

FIG. 33A depicts generation of islet specific EngTregs by FOXP3 HDR-editing and LV TCR transduction and includes a timeline of key steps for generating and enriching islet specific EngTregs from primary human CD4+ T cells. T cells were activated with CD3/CD28 beads on day 0 followed by transduction with lentiviral vectors (encoding islet specific TCRs on day 1). On day 7, flow cytometry was used to assess expression of islet specific TCR and Treg markers (mTCR CD25, CD127 CTLA-4 and ICOS). On day 10, islet specific EngTregs were enriched on LNGFR magnetic beads.

FIG. 33B depicts a diagram of FOXP3 locus (top); exons are represented by boxes. The AAV 6 donor template (bottom) was designed to insert the MND promoter, truncated LNGFR coding sequence and P2A (2A) sequence. After successful editing, the MND promoter drives expression of LNGFR and FOXP3.

FIG. 33C depicts representative flow plots (day 7, 4 days post editing) showing co expression of FOXP3 and LNGFR in edited cells (left panel), expression of mTCR, CD25, CD127, CTLA 4 and ICOS gated on LNGFR+ FOXP3+ cells from the left

FIG. 33D depicts representative flow plots (day 10, 7 days post editing) showing purity of LNGFR+ cells post-enrichment on anti-LNGFR magnetic beads. LNGFR− T cells were also collected to serve as controls for the in vitro suppression assays.

FIG. 33E depicts TCR expression and antigen specific proliferation of T cells transduced with islet TCR and include a schematic showing structure of lentiviral islet-specific TCR including variable region of human islet-specific TCR (huV-alpha and huV-beta) and constant region of murine TCR (muV-alpha and muV-beta).

FIG. 33F depicts validation of islet-specific TCR expression in human CD4+ T cells transduced with islet-specific TCRs. CD4+ T cells were isolated, activated with CD3/CD28 beads, and transduced with each lentiviral islet-specific TCR. Flow plots show mTCR expression in CD4+ T cells at 7 days post transduction using an antibody specific for the mouse TCR constant region.

FIG. 33G depicts proliferation of CD4+ T cells transduced with islet TCR in the presence of APC and their cognate peptide. TCR-transduced CD4+ T cells were labeled with cell trace violet and then co cultured with their cognate peptide (or irrelevant peptide) and APC (irradiated PBMC) for 4 days. Flow plots show cell proliferation as CTV dilution.

FIG. 33H depicts a comparison of mTCR expression levels in CD 4 T cells transduced with islet specific TCRs shown in FIG. 33F.

FIG. 34A depicts islet-specific EngTregs suppress antigen-induced Teff proliferation and includes a schematic of direct suppression of Teff by EngTregs with specificity for the same islet antigen. Shown here both the EngTregs and Teff are expressing T1D5-2 TCR, specific for IGRP₃₀₅₋₃₂₄.

FIG. 34B depicts representative histograms showing proliferation of T1D5-2 Teff (measured by CTV dilution) in the presence of either anti-CD3/CD28 antibody coated beads (top row) or cognate peptide (IGRP₃₀₅₋₃₂₄) and APC (bottom row) and the EF670-labelled EngTregs or controls. Histograms were gated on EF670− cells.

FIG. 34C depicts percent suppression of CD3/CD28 bead-induced Teff proliferation by poly EngTregs, LNGFR− T cells and islet-specific EngTregs either T1D5-2 (left), PPI76 (middle) or GAD65 (right).

FIG. 34D depicts percent suppression of antigen-induced Teff proliferation by poly EngTregs, LNGFR− T cells and islet-specific EngTregs either T1D5-2 (left), PPI76 (middle) or GAD65 (right); the cognate peptides were IGRP₃₀₅₋₃₂₄, PPI₇₆₋₉₀ and GAD65₂₆₅₋₂₈₄, respectively. For FIG. 34C and FIG. 34D, data are represented as mean±SD of three independent experiments using cells generated from three different healthy donors. P-values were calculated using a paired two-tailed Student t test (*P<0.05 and **P<0.01).

FIG. 34E depicts a timeline and key steps for production of islet specific EngTregs and Teff and the in vitro suppression assay. Teff were generated by TCR transduction of CD4+ T cells after activation with CD3/CD28 beads. Teff were expanded and harvested at day 15. Procedure for EngTregs production is described in FIG. 109A. Teff were co-cultured with or without EngTregs or LNGFR T cells in the presence of either APC (irradiated autologous PBMC) and various peptides or in the presence of CD3/CD28 beads. Teff and EngTregs or LNGFR− T cells were labeled with cell trace violet (CTV) and EF670 respectively, prior to co-culture. After 3 or 4 days of incubation, cells were harvested, stained, and analyzed by flow.

FIG. 34F depicts representative histograms showing proliferation of T1D4 Teff in the presence of CD3/CD28 beads, co-cultured with poly EngTregs or T1D4 EngTregs with different Treg:Teff ratios.

FIG. 34G depicts representative histograms showing proliferation of T1D4 Teff in the presence of cognate peptide (IGRP₂₄₁₋₂₆₀) and APC, performed in parallel with CD3/CD28 suppression assay in FIG. 34F.

FIG. 34H depicts percent suppression of CD3/CD28 bead-induced Teff proliferation by poly EngTregs and T1D4 EngTregs.

FIG. 34I depicts percent suppression of antigen-induced Teff proliferation by poly EngTregs and T1D4 EngTregs. For FIG. 34H and FIG. 34I, data are represented as mean±SD of five independent experiments using cells generated from four different healthy donors. P-values were calculated using a paired multiple t test (***P<0.005).

FIG. 35A depicts islet-specific EngTregs suppress antigen-induced Teff cytokine production and includes representative flow plots showing Teff cytokine production (TNF-α, IL-2 and IFN-γ) and activation (CD25 expression) in an antigen-specific suppression assay. T1D5-2 Teff in the presence of T1D5-2 cognate peptide IGRP₃₀₅₋₃₂₄ and APC were cultured alone or with polyclonal EngTregs, LNGFR− T cells, or T1D5-2 EngTregs.

FIG. 35B depicts percent suppression of antigen-induced T1D5-2 Teff production of TNFα (left) IL-2 (middle) and IFNγ (right) by poly EngTregs LNGFR− T cells and islet-specific T1D5-2 EngTregs.

FIG. 35C depicts percent suppression of antigen-induced T1D5-2 Teff expression of CD25 by poly EngTregs, LNGFR− T cells and islet-specific T1D5-2 EngTregs. For FIG. 35B and FIG. 35C, data are represented as mean±SD of four independent experiments using cells generated from four different healthy donors. P values were calculated using a paired two tailed Student t test (*P<0.05, **P<0.01 and ***P<0 001).

FIG. 36A depicts islet-specific EngTregs suppress bystander Teff proliferation and includes a schematic of bystander suppression of Teff by EngTregs with specificity for different islet antigens. Shown here the EngTregs expresses T1D4 TCR specific for IGRP₂₄₁₋₂₆₀, and the Teff express T1D5-2 TCR specific for IGRP₃₀₅₋₃₂₄.

FIG. 36B depicts representative histograms showing proliferation of T1D5-2 Teff (measured by CTV dilution) in the presence of either IGRP₃₀₅₋₃₂₄ peptide (top panel) or mixture of IGRP₃₀₅₋₃₂₄ and IGRP₂₄₁₋₂₆₀ peptides (bottom row) plus APC and either T1D5-2 EngTregs, T1D4 EngTregs or poly EngTregs. EngTregs were labeled with EF670 and histograms were gated on EF670− cells.

FIG. 36C depicts percent suppression of T1D5-2 Teff proliferation by poly EngTregs, T1D5-2 EngTregs or T1D4 EngTregs in the presence of a mixture of IGRP₃₀₅₋₃₂₄ and IGRP₂₄₁₋₂₆₀ peptides plus APC.

FIG. 36D depicts representative histograms showing proliferation of T1D5-2 Teff (measured by CTV dilution) in the presence of either IGRP₃₀₅₋₃₂₄ peptide (top panel) or mixture of IGRP₃₀₅₋₃₂₄ and GAD₂₆₅₋₂₈₄ peptides (bottom row) plus APC and poly EngTregs and GAD265 EngTregs. EngTregs were labeled with EF670 and histograms were gated on EF670− cells.

FIG. 36E depicts percent suppression of proliferation of T1D5-2 Teff by poly EngTregs or GAD265 EngTregs in the presence of APC and mixture of IGRP₃₀₅₋₃₂₄ and GAD₂₆₅₋₂₈₄ peptides plus APC.

FIG. 36F depicts percent suppression of T1D5-2 Teff cytokine production by T1D5-2 Teff by poly EngTregs, T1D5-2 EngTregs or T1D4 EngTregs in the presence of APC and mixture of IGRP₃₀₅₋₃₂₄ and IGRP₂₄₁₋₂₆₀ peptides.

FIG. 36G depicts percent suppression for T1D5-2 Teff CD25 expression by poly EngTregs, T1D5-2 EngTregs or T1D4 EngTregs in the presence of APC and mixture of IGRP₃₀₅₋₃₂₄ peptide and IGRP₂₄₁₋₂₆₀ peptide. For FIG. 36C, FIG. 36E, FIG. 36F and FIG. 36G, data are provided as the mean±SD of three independent experiments using cells generated from three different healthy donors. P values were calculated using a paired two tailed Student t test (* P<0.05, P<0.01 and P<0.005). LNGFR− T cells with either T1D5-2 TCR or T1D4 TCR were used as a negative control for all three experiments and did not show any significant suppression.

FIG. 36H depicts islet-specific EngTregs show comparable suppression on CD3/CD28 bead induced Teff proliferation and includes representative flow plots showing mTCR expression in FOXP3-edited cells transduced with no TCR (-), T1D4 TCR or T1D5-2 TCR. Edited cells were stained at day 7 and were gated on Live, CD3+, CD4+, LNGFR+, FOXP3+.

FIG. 36I depicts representative histograms showing proliferation of T1D5-2 Teff in CD3/CD28 bead suppression assay performed in parallel with bystander suppression assay in FIG. 36B and FIG. 36C. T1D5-2 Teff were incubated with CD3/CD28 beads with no Treg (-), polyclonal EngTregs, T1D5-2 EngTregs, or T1D4 EngTregs.

FIG. 36J depicts percent suppression of CD3/CD28 bead induced-T1D5-2 Teff proliferation by poly EngTregs, T1D5-2 EngTregs, or T1D4 EngTregs in (FIG. 36I).

FIG. 36K depicts representative histograms showing T1D5-2 Teff proliferation in CD3/CD28 bead suppression assay performed in parallel with bystander suppression assay in FIG. 36D and FIG. 36E. T1D5-2 Teff were incubated with CD3/CD28 beads with no Treg (-), poly EngTregs, or GAD265 EngTregs.

FIG. 36L depicts percent suppression of CD3/CD28 bead induced-T1D5-2 Teff proliferation by poly EngTregs or GAD265 EngTregs in FIG. 36K. For FIG. 36J and FIG. 36L, data are represented as the mean±SD of three independent experiments using cells generated from three different healthy donors. P-values were calculated using a paired two-tailed Student t test.

FIG. 36M depicts representative histograms showing islet specific EngTregs suppression of bystander Teff cytokine production and includes representative histograms showing T1D5-2 Teff production of TNFα in antigen-specific bystander suppression assay. Columns are the same as those labelled in FIG. 36M.

FIG. 36N depicts representative histograms showing T1D5-2 Teff production of IL2 in antigen-specific bystander suppression assay. Columns are the same as those labelled in FIG. 36M.

FIG. 36O depicts representative histograms showing T1D5-2 Teff production of IFNγ in antigen-specific bystander suppression assay. Columns are the same as those labelled in FIG. 36M.

FIG. 36P depicts representative histograms showing T1D5-2 Teff expression of CD25 in antigen-specific bystander suppression assay. Columns are the same as those labelled in FIG. 36M. For FIGS. 36M-36P, T1D5-2 Teff were co-cultured with no Treg poly EngTregs, T1D5-2 EngTregs or T1D4 EngTregs in the presence of APC and either IGRP₃₀₅₋₃₂₄ peptide alone or a mixture of IGRP₃₀₅₋₃₂₄ and IGRP₂₄₁₋₂₆₀ peptides.

FIG. 37A depicts islet-specific EngTregs suppress polyclonal islet-specific Teff derived from T1D PBMC, and includes a timeline and key steps for production of islet-specific EngTregs, polyclonal islet specific Teff, and monocyte derived DC (mDC) from PBMC from T1D donor, and the in vitro suppression assay.

FIG. 37B depicts representative histograms showing proliferation of polyclonal islet Teff (measured by CTV dilution) in the presence of either CD3/CD28 beads (top panel) or islet-specific antigens (9 islet specific peptides monocyte derived DC (mDC)) (bottom row) and either T1D2 EngTregs, 4.13 EngTregs, LNGFR− T cells or poly EngTregs. EngTregs were labeled with EF670 and histograms were gated on EF670− cells

FIG. 37C depicts percent suppression of CD3/CD 28 induced proliferation of polyclonal islet Teff by T1D2 EngTregs, 4.13 EngTregs, LNGFR− T cells or poly EngTregs.

FIG. 37D depicts percent suppression of antigen-induced proliferation of polyclonal islet Teff by T1D2 EngTregs, 4.13 EngTregs, LNGFR− T cells or poly EngTregs. Antigen stimulation by pool of 9 islet specific peptides in the presence of mDC. Data are provided as the mean±SD of three independent experiments using cells generated from three different T1D donors. P values were calculated using a paired two-tailed Student t test (* P<0.05 **P<0.01 and ***P<0.0001). Co-culture in the presence of mDC and DMSO was included as a negative control and showed no significant proliferation of Teff.

FIG. 37E depicts expansion of islet-specific T cells of multiple specificities derived from T1D PBMC, and includes a timeline and key steps of peptide stimulation to expand islet-specific T cells. CD4+CD25− T cells isolated from T1D donor were stimulated with HLA-DR0401 restricted 9 islet peptides specific for GAD65 (5), IGRP (3), and PPI (1) and irradiated autologous APC (CD4−CD25+) followed by tetramer staining at day 12 to 14. T cells were cultured without IL-2 until day 7, and then expanded with IL-2 at 2-3 days of interval.

FIG. 37F depicts representative flow plots showing tetramer+ T cells specific for individual antigenic peptides. Staining with no tetramer was included as a negative staining result. Cells were gated on CD4+ T cells and each percentage indicates the level of tetramer staining above background.

FIG. 37G depicts percent tetramer+ population in CD4+ T cells measured and combined from 5 different experiments using 3 different T1D donors after 12-14 days of in vitro peptide stimulation. Each bar indicates the percentage of CD4+ T cells specific for each islet antigenic peptide. Each dot represents a different experiment.

FIG. 37H depicts islet-specific EngTregs are superior at suppressing polyclonal islet-specific Teff than tTreg, and includes representative histograms showing proliferation of polyclonal islet Teff in the presence of either anti-CD3/CD28 antibody coated beads (Top row) or mDC and a pool of 9 islet-specific peptides (Bottom row) performed in parallel. Polyclonal islet Teff were cultured with no Treg (-), T1D2 LNGFR−, T1D2 EngTregs, or tTreg. tTreg were sorted by CD4+CD25+CD127− and cultured in the same way as EngTregs. tTreg were activated with CD3/CD28 beads for 2 days, expanded, and harvested at day 10. All the cells used for suppression assays are autologous and prepared from a T1D donor. Co-culture in the presence of monocyte-derived DC (mDC) and DMSO was included as a negative control and showed no significant proliferation of Teff.

FIG. 371 depicts percent suppression of CD3/CD28 bead induced-proliferation of polyclonal islet Teff by T1D2 LNGFR−, T1D2 EngTregs, or tTreg.

FIG. 37J depicts percent suppression of antigen induced-proliferation of polyclonal islet Teff by T1D2 LNGFR−, T1D2 EngTregs, tTreg.

FIG. 38A depicts islet specific EngTregs inhibit APC maturation and utilize both cell contact dependent and independent mechanisms to suppress Teff, and include a schematic of transwell suppression assay: upper and lower chamber separated by permeable membrane.

FIG. 38B depicts percent suppression of proliferation of polyclonal islet specific Teff measured by CTV dilution in lower chamber (left panel) or upper chamber (right panel). Polyclonal islet Teff were co cultured with T1D2 EngTregs as a positive control. Data are provided as the mean±SEM of three independent experiments using cells generated from three different T1D donors. ***P<0.001, **P<0.01, *P<0.05, as determined by paired t-test.

FIG. 38C depicts a timeline and key steps for DC maturation and APC modulation assay.

FIG. 38D depicts normalized CD86 MFI on mDC. Autologous matured mDC with HLA DR0401 were co cultured with T1D2 EngTregs or LNGFR− T cells in the presence of IGRP₃₀₅₋₃₂₄ peptide for 2 days. MFI of CD86 on DCs were normalized by MFI of DC only condition. Data are provided as the mean SD of three independent experiments using cells generated from three different healthy donors. *P<0.05, as determined by paired t-test.

FIG. 38E depicts representative histograms showing proliferation of polyclonal islet-specific Teff co-cultured with islet specific antigens (10Ags including IGRP₃₀₅₋₃₂₄) and mDC in the presence of T1D2 EngTregs with addition of exogenous human IL2 (0.1 IU/ml). Teff and EngTregs were labeled with CTV and EF670, respectively, before the co-culture and CTV dilution was measured as proliferation.

FIG. 38F depicts percent suppression on Teff proliferation shown in FIG. 38E. % Suppression was calculated separately in the absence or presence of exogenous human IL2. Data are provided as the mean±SEM of three independent experiments using cells generated from three different T1D donors. Ns, not significant, as determined by paired t-test.

FIG. 38G depicts islet-specific EngTregs show both contact dependent and independent bystander suppression, and includes generation of polyclonal islet-specific Teff to investigate mechanisms for bystander suppression by islet specific EngTregs. CD4+CD25− T cells isolated from T1D donor were stimulated with HLA-DR0401 restricted 9 islet peptides specific for GAD65₁₁₃₋₁₃₂, GAD₂₆₅₋₂₈₄, GAD₂₇₃₋₂₉₂, GAD₃₀₅₋₃₂₄, IGRP₁₇₋₃₆, IGRP₂₄₁₋₂₆₀, PPI₇₆₋₉₀, ZNT8₂₆₆₋₂₈₅ and irradiated autologous APC (CD4−CD25+) followed by tetramer staining at day 14 or 15. T1D2 TCR specific IGRP₃₀₅₋₃₂₄ peptide was excluded for Teff expansion. Representative flow plots showing tetramer T cells specific for individual antigenic peptides. Staining with no tetramer was included as a negative staining result. Cells were gated on CD4+ T cells and each percentage indicates the level of tetramer staining above background.

FIG. 38H depicts percent tetramer population in CD4+ T cells measured and combined from 3 different T1D donors after 14-15 days of in vitro peptide stimulation. Each bar indicates the percentage of CD4+ T cells specific for each islet antigenic peptide. Each dot represents a different T1D donor.

FIG. 38I depicts representative histograms showing proliferation of polyclonal islet-specific Teff at lower well (top) or upper well (lower). mDC loaded with a pool of islet peptides (10 Ags including IGRP₃₀₅₋₃₂₄) were plated in both lower and upper well. Polyclonal islet-specific Teff or/and T1D2 EngTregs were added in lower or/and upper well as indicated.

FIG. 38J depicts islet-specific EngTregs inhibit CD86 expression on dendritic cells, and includes autologous monocytes restricted to HLA-DR0401 were matured into DC with GM-CSF/IL-4 and IFNg/CL075. Matured DC were co-cultured with CTV-labeled EngTregs or LNGFR− T cells expressing islet-TCR in the presence of cognate peptide. After 2 days of incubation, cells were harvested, stained, analyzed by flow.

FIG. 38K depicts representative data showing MFI of CD86 on DC co-cultured with T1D2 EngTregs or LNGFR− T cells.

FIG. 38L depicts bar histograms showing normalized expression level of CD86 on DC co-cultured with T1D4 EngTregs or LNGFR− T cells in the presence of IGRP₂₄₁₋₂₆₀ peptide (left) or with PP176 EngTregs or LNGFR− T cells in the presence of PPI₇₆₋₉₀ peptide (right).

FIG. 38M depicts mTCR expression in FOXP3-edited cells transduced with no TCR (poly), T1D2 TCR or 4.13 TCR. Edited cells were stained at day 7 and were gated on Live, CD3+, CD4+, LNGFR+. LNGFR+ (EngTregs) and LNGFR−T cells enriched using anti-LNGFR magnetic beads were used in suppression assay shown in FIGS. 194A-194D.

FIG. 38N depicts representative histograms showing proliferation of polyclonal islet Teff in the presence of either CD3/CD28 beads (Top row) or mDC and a pool of 9 islet-specific peptides (Bottom row) performed in parallel. Polyclonal islet Teff were cultured with no Treg (-), T1D2 LNGFR−, T1D2 EngTregs, or tTreg. tTreg were sorted by CD4+CD25+CD127− and cultured in the same way as EngTregs. tTreg were activated with CD3/CD28 beads for 2 days, expanded, and harvested at day 10. All the cells used for suppression assays are autologous and prepared from a T1D donor. Co-culture in the presence of monocyte-derived DC (mDC) and DMSO was included as a negative control and showed no significant proliferation of Teff (data not shown).

FIG. 38O depicts percent suppression of CD3/CD28 bead induced-proliferation of polyclonal islet Teff by T1D2 LNGFR−, T1D2 EngTregs, or tTreg.

FIG. 38P depicts Percent suppression of antigen induced-proliferation of polyclonal islet Teff by T1D2 LNGFR−, T1D2 EngTregs, tTreg. This is representative data from two independent experiments.

FIG. 39A depicts a graph showing peptide dose response of T cells expressing T1D2, T1D4, or PPI76 TCR. CD4+ T cells transduced with T1D2, T1D4, or PPI76 TCR were co-cultured with APC in the presence of various concentration of their cognate peptide, IGRP₃₀₅₋₃₂₄, IGRP₂₄₁₋₂₆₀, PPI₇₆₋₉₀, respectively for 4 days. Representative of three independent experiments.

FIG. 39B depicts percent suppression of antigen-induced proliferation of polyclonal islet Teff by T1D2, T1D4, or PPI76 EngTregs. Data are provided as the mean±SEM of four independent experiments using cells generated from four different T1D donors. P-values were calculated using a paired two-tailed Student t test (*P<0.05 and **P<0.01).

FIG. 39C depicts a graph showing peptide dose response of T cells expressing T1D2, T1D5-1, or T1D5-2 TCR. CD4+ T cells transduced with T1D2, T1D5-1 or T1D5-2 TCR were co-cultured with APC in the presence of various concentration of their cognate peptide, IGRP₃₀₅₋₃₂₄ for 4 days. Representative of three independent experiments. For dose response of T cells in A and C, T cells were labeled with CTV before the co-culture and cell proliferation was measured by CTV dilution.

FIG. 39D depicts percent suppression of antigen-induced proliferation of polyclonal islet Teff by T1D2, T1D5-1, or T1D5-2 EngTregs. Data are provided as the mean±SEM of four independent experiments using cells generated from four different T1D donors. P-values were calculated using a paired two-tailed Student t test (*P<0.05). For suppression assays in B and D, data are normalized by suppressive activity obtained from suppression assay set up in parallel using CD3/CD28 beads. Suppressive activity was calculated as (% suppression/% the lowest suppression). Normalization of antigen-specific suppression was calculated as (% suppression from antigen-specific assay/suppressive activity).

FIG. 39E depicts representative flow plots showing mTCR expression in FOXP3 edited cells transduced with T1D2 T1D4 or PPI76 TCR.

FIG. 39F depicts a comparison of mTCR expression levels shown in FIG. 39E. Edited cells were stained at day 7 and were gated on Live, CD3+ CD4+ LNGFR+ FOXP3+ Enriched LNGFR+ cells EngTregs expressing T1D2 T1D4 or PPI76 TCR were used in suppression assays.

FIG. 39G depicts representative histograms showing proliferation of polyclonal islet Teff in the presence of islet specific antigens (10 islet specific peptides+monocyte derived DC)mDC)) and either T1D2 T1D4 or PP176 EngTregs.

FIG. 39H depicts representative flow plots showing mTCR expression in FOXP3 edited cells transduced with T1D2 T1D5-1 or T1D5-2 TCR.

FIG. 39I depicts a comparison of mTCR expression levels shown in FIG. 39H. Edited cells were stained at day 7 and were gated on Live, CD3+ CD4+ LNGFR+ FOXP3+. Enriched LNGFR+ cells (EngTregs) expressing T1D2 T1D5-1 or T1D5-2 TCR were used in suppression assays.

FIG. 39J depicts representative histograms showing proliferation of polyclonal islet Teff in the presence of islet specific antigens (10 islet specific peptides+mDC) and either T1D2 T1D5-1 T1D5-2 EngTregs Polyclonal islet Teff and EngTregs were labeled with CTV and EF 670 respectively and cell proliferation was measured as CTV dilution.

FIG. 40A depicts generation of murine islet-specific EngTregs by gene editing in BDC2.5 CD4+ T cells and includes a diagram of AAV 5 packaged, MND LNGFR p2A knock-in donor template for use in FOXP3 HDR editing. Exons are represented by numbered boxes, FOXP3 homology arms are indicated. After successful editing, the MND promoter drives expression of endogenous murine FOXP3 protein and cis-linked LNGFR surface expression.

FIG. 40B depicts a schematic showing the experimental timeline for FOXP3 gene editing, cell analysis, and enrichment of edited LNGFR cells.

FIG. 40C depicts representative flow plots (from one of four independent experiments) showing LNGFR expression in mock-edited control cells (left) and cells edited with RNP and AAV donor template pre- (middle) and post-LNGFR+ column-enrichment (right).

FIG. 40D depicts representative flow cytometry histogram (from one of two independent experiments) showing the expression of Treg associated markers for the indicated cell populations.

FIG. 40E depicts bar graphs showing MFI for Treg associated markers on EngTregs, or mock edited cells. Error bars show ±SD. P values were calculated using an unpaired T test comparing EngTregs and mock edited cells.

FIG. 40F depicts a schematic of in vitro suppression assays performed using BDC2.5 CD4+ Teff cells and mock control, BDC2.5 tTreg or EngTregs cells.

FIG. 40G depicts representative flow plots (from one of three independent experiments) showing CTV labeled BDC2.5 CD4+ Teff co-cultured with the indicated cells 4 days post stimulation.

FIG. 40H depicts a graph showing the percent suppression of BDC2.5 CD4+ Teff proliferation by the indicated Treg co culture at varying ratios of Teff Treg suppression 100 normalized suppression] normalized suppression 100/proliferation of Teff only condition× Teff proliferation in the presence of Treg.

FIG. 41A depicts islet specific, but not polyclonal, EngTregs prevent T1D onset in vivo, and includes a schematic showing the experimental timeline for murine diabetes prevention studies.

FIG. 41B depicts a graph showing diabetes-free survival of recipient NSG mice after infusion of islet-specific Teff in the presence of the indicated co-transferred cell populations. Data are combined from two independent experiments; ****, P<0.0001, calculated using a log rank (Mantel-Cox) test comparing the BDC2.5 tTreg or EngTreg groups vs. the mock-edited control group.

FIG. 41C depicts at left panel including representative flow plots of lymphocytes isolated from the pancreas in diabetes-free NSG recipient mice on day 49 after BDC2.5 CD4 Teff infusion. Upper and lower panels show data for recipients of BDC2.5 tTreg vs. BDC2.5 EngTreg, respectively. Predecessor gates for flow panels are indicated at the top of each column. Right panel, histograms show FOXP3 expression within the indicated (color coded) flow gates.

FIG. 41D depicts representative flow plots showing LNGFR expression in the indicated (top of column) edited CD4 T cells derived from NOD (polyclonal; top row) and NOD BDC2.5 mice (islet specific; bottom row).

FIG. 41E depicts a graph showing diabetes-free survival in recipient NSG mice following infusion of islet specific Teff in the presence co transferred mock edited, or polyclonal or islet specific EngTregs or tTreg cells. Combined data from two independent experiments are shown; **** P<0.0001, determined using the Mantel Cox log rank test comparing BDC2.5 tTreg or EngTregs vs. polyclonal tTreg or EngTregs, respectively. All flow plots are representative of at least two independent experiments.

FIG. 41F depicts experimental schematic for diabetes prevention studies using diabetogenic NOD splenocytes.

FIG. 41G depicts a graph showing diabetes-free survival of recipient NSG mice after infusion of diabetogenic NOD Teff in the presence or absence of co-transferred BDC2.5 EngTregs. Data shown are from a single experiment; **, P<0.005, calculated using a log-rank (Mantel-Cox) test comparing the BDC2.5 EngTregs group vs. recipients of only diabetogenic NOD Teff.

FIG. 41H depict representative histological images of single representative islets showing H&E (left panels), anti-CD 3 (middle panels), and insulin staining (right panels) Results are shown for representative NSG animals treated with diabetogenic NOD splenocytes alone Upper panels Mouse tissue harvested at time of meeting euthanasia criteria for diabetes) vs co delivery of diabetogenic NOD splenocytes and BDC 2 5 EngTregs Middle panels Mouse 6 surviving until study end without hyperglycemia) and, in comparison with an untreated, age matched control NSG mouse (Mouse 22, lower panels harvested at study end) All photos show 20× images embedded marker represents 80 micrometers.

FIG. 41I depicts a summary of histologic findings. Histology was performed on two animals from each of the indicated experimental treatment groups L 1 and L 2 represent step sections from the same tissue block. All islets within each H&E stained section were evaluated for degree of lymphocytic insulitis as judged by accumulation of lymphoid cells within and/or surrounding islets. Individual islets across both sections were then assigned to one of the categories of severity (normal to severe insulitis) and the numbers (in columns 3-6 indicate the area (islets)/mm 2 of the total pancreatic section area with the indicated level of insulitis. Separate matched tissue sections were evaluated for the presence of insulin using immunohistochemistry (IHC) and the total numbers of positively stained islets from each section were assigned to the ‘Insulin positive islets by IHC’ category below (column 7 Because sections vary in area, the islet counts from each animal and section were normalized by expressing total numbers of islets assigned to each category as islets/mm² of the pancreatic tissue section Inflammatory involvement of the pancreatic interstitium was made using anti-CD3 stained sections and graded on a scale of 0 (normal) to 3+(marked relative to other sections column 8).

### DETAILED DESCRIPTION

Aspects of the disclosure relate to methods and compositions for producing engineered Treg cells that have (i) stable suppressive function, e.g., by stabilizing FoxP3 expression; (ii) specificity for a type 1 diabetes (T1D)-associated antigen; and (iii) exhibit IL-2-like signal transduction in the presence of rapamycin. Embodiments relate to insertion of two nucleic acids into targeted loci of a cell genome. A first nucleic acid, inserted into the TRAC locus, encodes, under control of a strong constitutive (e.g., MND) promoter: (a) a first component of a heterodimerizable protein complex that provides intracellular IL-2 signal transduction in the presence of rapamycin, comprising an extracellular FK506-binding protein 12 (FKBP) domain covalently linked to an IL-2Rγ transmembrane and cytoplasmic domain; (b) a TCRβ chain of a TCR specific to a T1D-associated peptide of IGRP; and (c) in-frame with the endogenous TCRα constant region, such that a TCRα chain which, together with the TCRβ chain forms the IGRP-specific TCR, is expressed from the TRAC locus. A second nucleic acid, inserted into the FOXP3 locus, downstream from Treg-specific demethylated region, encodes, under control of a strong constitutive promoter (e.g., MND) promoter: (a) second component of the heterodimerizable protein complex for intracellular IL-2 signal transduction, comprising an extracellular FKBP-rapamycin-binding (FRB) domain covalently linked to an IL-2Rβ transmembrane and cytoplasmic domain; (b) a soluble FRB domain for adsorbing intracellular rapamycin to limit mTOR inhibition; and (c) at least a portion of the endogenous first coding exon of FOXP3, such that the inserted promoter controls expression of FoxP3 independently of the endogenous promoter and TSDR-mediated regulation. Thus, the dual-edited cells described herein are T1D-associated antigen-specific Tregs, which both retain a stable suppressive phenotype in inflammatory environments (e.g., an inflamed pancreas), and may be expanded in a controllable manner in the presence of rapamycin.

### Methods for Producing Genetically Modified Cells

Some aspects of the disclosure relate to methods of producing a genetically modified cell by introducing into the cell two nucleic acids, one with homology to the TRAC locus, and another with homology to the FOXP3 locus of the cell, such that both loci are edited by insertion of the nucleic acids into respective loci. The first nucleic acid, targeting the TRAC locus, comprises 5′ and 3′ homology arms to direct insertion of the nucleic acid into the TRAC locus (e.g., by homology-directed repair (HDR) following cleavage of a DNA sequence in the TRAC locus by a nuclease). The second nucleic acid, targeting the FOXP3 locus, comprises 5′ and 3′ homology arms to direct insertion of the nucleic acid into the FOXP3 locus (e.g., by HDR following cleavage of a DNA sequence in the FOXP3 locus by a nuclease). Insertion of both nucleic acids into separate loci of the cell results in a dual-edited cell (i.e., a cell having inserted nucleic acids at two distinct loci).

In embodiments of the methods described herein, the nucleic acid targeted for insertion into the TRAC locus comprises a promoter that is operably linked to: (i) a nucleotide sequence encoding a first chemically induced signaling complex (CISC) component comprising: (a) an extracellular binding domain comprising or derived from an FK506-binding protein 12 (FKBP), (b) a transmembrane domain comprising or derived from an IL-2Rγ transmembrane domain, and (c) an intracellular signaling domain comprising or derived from an IL-2Rγ cytoplasmic domain; (ii) a nucleotide sequence encoding a full-length TCRβ chain; and (iii) a nucleotide sequence encoding at least a portion of a TCRα chain. The nucleotide sequence encoding the heterologous TCRα is inserted in-frame with an endogenous sequence encoding an endogenous TCRα portion (e.g. a TCRα constant domain), such that translation of the expressed mRNA produces a TCRα chain that associates with the heterologous TCRβ chain to form a TCR. Because the antigen-binding regions of the TCRα chain are encoded by the inserted nucleic acid, the specificity of the TCR is governed by the inserted nucleic acid. In the methods described herein, the TCR encoded by the inserted nucleic acid binds to a T1D-associated antigen. Following insertion into the TRAC locus, the promoter initiates transcription (and thereby promotes expression) of the operably linked sequences, such that the FKBP-IL2Rγ CISC component, and a T1D-associated antigen-specific TCR formed by the heterologous TCRβ chain and TCRα chain comprising the heterologous portion encoded by the inserted nucleic acid, are expressed from the TRAC locus.

In embodiments of the methods described herein, the nucleic acid targeted for insertion into the FOXP3 locus comprises a promoter that is operably linked to: (i) a nucleotide sequence encoding a first chemically induced signaling complex (CISC) component comprising: (a) an extracellular binding domain comprising or derived from an FKBP-rapamycin-binding (FRB) domain of mTOR, (b) a transmembrane domain comprising or derived from an IL-2Rβ transmembrane domain, and (c) an intracellular signaling domain comprising or derived from an IL-2Rβ cytoplasmic domain; (ii) a nucleotide sequence encoding a cytosolic FRB domain that lacks a transmembrane domain; and (iii) a 3′ homology arm with homology to a sequence in the FOXP3 locus that is downstream from the Treg-specific demethylated region in the FOXP3 locus (e.g., homology to a sequence within or up to 2,000 nucleotides upstream from exon 2, the first coding exon of the FOXP3 gene). Insertion in this manner downstream from the TSDR, which destabilizes FOXP3 expression in inflammatory conditions, allows the inserted promoter to initiate transcription of FoxP3-encoding mRNA independently of the endogenous FOXP3 promoter, which is upstream from the TSDR. Following insertion into the FOXP3 locus, the promoter initiates transcription of the operably linked sequences, such that the FRB-Il2Rβ CISC component, cytosolic FRB component, and FoxP3 are expressed from the FOXP3 locus.

Following insertion of both nucleic acids into the TRAC and FOXP3 loci, respectively, the dual-edited cell stably expresses: (i) first and second CISC components that form a heterodimer in the presence of rapamycin, resulting in IL-2R signal transduction via dimerization of the cytoplasmic IL-2Rβ and IL-2Rγ domains; (ii) a cytosolic FRB domain that binds intracellular rapamycin, preventing its interaction with mTOR; (iii) FoxP3, providing for a stable Treg phenotype; and (iv) a TCR specific to a T1D-associated antigen. Thus, the methods described herein provide for stable Treg cells with T1D-associated antigen specificity, which can be induced to proliferate using rapamycin. Moreover, separation of the nucleotide sequences encoding first and second CISC components onto distinct nucleic acids allows rapamycin to induce proliferation selectively in cells expressing both CISC components (and thus expressing the T1D antigen-specific TCR and FoxP3 due to insertion of both nucleic acids). Thus, dual-edited cells may readily be selected and proliferated in vitro to produce a population of stable Treg cells having T1D-associated antigen specificity for treating T1D. Additionally, engraftment and proliferation of such stable Treg cells may be supported in vivo by administering rapamycin to a subject.

### Promoters

Nucleic acids for targeted insertion into cell genomes by methods described herein each comprise a promoter operably linked to one or more nucleotide sequences on the nucleic acid. A promoter is “operably linked” to a sequence if it is capable of initiating transcription of the operably linked sequence (e.g., by recruitment of RNA polymerase). The promoters of the first and second nucleic acids may be any promoter known in the art. In some embodiments, the heterologous promoter on the introduced nucleic acid is active, promoting transcription of RNA, even under pro-inflammatory conditions. In some embodiments, the promoter is a constitutive promoter. Constitutive promoters may be strong promoters, which promote transcription at a higher rate than an endogenous promoter, or weak promoters, which promote transcription at a lower rate than a strong or endogenous promoter. In some embodiments, the constitutive promoter is a strong promoter. In some embodiments, the heterologous promoter is an inducible promoter. Inducible promoters promote transcription of an operably linked sequence in response to the presence of an activating signal, or the absence of a repressor signal. In some embodiments, the inducible promoter is inducible by a drug or steroid.

In some embodiments, the promoters of the first and second nucleic acids delivered to the cell are different promoters. In other embodiments, the first and second nucleic acid both comprise the same promoter. In some embodiments, the first and second nucleic acid both comprise an MND promoter. In embodiments where the first and second nucleic acid both comprise the same promoter, the promoter sequences may be identical between both nucleic acids. Alternatively, the promoter sequence of the first nucleic acid may comprise one or more mutations (e.g., insertions, deletions, substitutions) relative to the promoter sequence of the second nucleic acid. In some embodiments, the MND promoter of the first and/or second nucleic acid comprises at least 90% sequence identity to the nucleic acid sequence of SEQ ID NO: 220. In some embodiments, the MND promoter of the first and/or second nucleic acid comprises at least 95% sequence identity to the nucleic acid sequence of SEQ ID NO: 220. In some embodiments, each of the first and second nucleic acids comprises an MND promoter having the nucleic acid sequence of SEQ ID NO: 220.

In some embodiments, a STOP codon is present upstream or within the first five nucleotides of the promoter on the first nucleic acid for insertion into the TRAC locus. In some embodiments, a STOP codon is present upstream or within the first five nucleotides of the promoter on the second nucleic acid for insertion into the FOXP3 locus. The presence of a STOP codon upstream from, within, or overlapping with the first five nucleotides of the promoter is expected to terminate translation of mRNAs that may be transcribed from an endogenous promoter upstream in the modified TRAC or FOXP3 locus, thereby inhibiting expression of inserted coding sequences (e.g., encoding CISC components, heterologous TCRβ or TCRα chains, or FoxP3) under control of the endogenous promoter. In some embodiments, the STOP codon is in-frame with one or more upstream START codons, such that mRNA produced following transcription from the endogenous upstream promoter is not translated past the STOP codon.

### Chemically Induced Signaling Complex (CISC)

Embodiments of the methods for producing genetically modified cells described herein, each nucleic acid inserted into the cell genome comprises a nucleotide sequence encoding a chemically induced signaling complex (CISC) component, each CISC component comprising an extracellular domain that binds rapamycin, a transmembrane domain, and an intracellular domain comprising or derived from an interleukin-2 receptor (IL-2R) cytoplasmic domain. In some embodiments, the first nucleic acid (for insertion into the TRAC locus) encodes a first CISC component comprising (i) an extracellular binding domain comprising an FK506-binding protein 12 (FKBP) domain, (ii) a transmembrane domain comprising or derived from an IL-2Rγ transmembrane domain, and (iii) an intracellular domain comprising or derived from an IL-2Rγ cytoplasmic domain; and the second nucleic acid (for insertion into the FOXP3 locus) encodes a first CISC component comprising (i) an extracellular binding domain comprising an FKBP-rapamycin-binding domain, (ii) a transmembrane domain comprising or derived from an IL-2Rβ transmembrane domain, and (iii) an intracellular domain comprising or derived from an IL-2Rβ cytoplasmic domain. A domain of a CISC component (e.g., transmembrane domain of the first CISC component) is “derived from” a given domain of an IL-2R polypeptide (e.g., IL-2Rγ) if it comprises at least 90% sequence identity to a wild-type (naturally occurring) amino acid sequence of the domain (e.g., a naturally occurring IL-2Rγ transmembrane domain).

Expression of CISC components in a cell allows selective induction of IL-2 signal transduction in a cell by manipulation of the presence and/or concentration of the rapamycin. Such controllable induction of signaling allows, for example, selective expansion of cells expressing both CISC components, where the IL-2 signal transduction event results in proliferation of the cell. In some embodiments, where two nucleic acids, each encoding a different CISC component, are introduced into the cell, such selective expansion allows for selection of cells that contain both nucleic acids, as contacting a cell comprising only one CISC component with rapamycin would not induce dimerization with the absent second CISC component, and thus not lead to IL-2 signal transduction.

Non-limiting examples of intracellular signaling domains include IL-2Rβ and IL-2Rγ cytoplasmic domains and functional derivatives thereof. In some embodiments, an intracellular signaling domain of the first CISC component comprises an IL-2Rγ domain or a functional derivative thereof, and an intracellular signaling domain of a second CISC component comprises an IL-2Rβ cytoplasmic domain or a functional derivative thereof. In some embodiments, dimerization of the first and second CISC components induces phosphorylation of JAK1, JAK3, and/or STAT5 in the cell. In some embodiments, dimerization of the first and second CISC components induces proliferation of the cell.

Non-limiting examples of transmembrane domains include IL-2Rβ and IL-2Rγ transmembrane domains and functional derivatives thereof. In some embodiments, the transmembrane domain of a CISC component is derived from the same protein as the intracellular signaling domain of the CISC component (e.g., a CISC component comprising an IL-2Rβ intracellular domain comprises an IL-2Rβ transmembrane domain). In some embodiments, one CISC component comprises an IL-2Rβ transmembrane domain, and the other CISC component comprises an IL-2Rγ transmembrane domain.

Non-limiting examples of extracellular binding domains capable of binding to rapamycin include an FK506-binding protein (FKBP) domain and an FKBP-rapamycin-binding (FRB) domain. FKBP and FRB domains are capable of binding to rapamycin, such as those described below, to form a heterodimer. In some embodiments, an extracellular binding domain of one CISC component comprises an FKBP domain, and an extracellular binding domain of the other CISC component comprises an FRB domain. In some embodiments, the CISC components form a heterodimer in the presence of rapamycin. In some embodiments, the FRB domain comprises a threonine at a position corresponding to amino acid 2098 of wild-type mTOR having the amino acid sequence of SEQ ID NO: 236. Mutation of this amino acid increases the affinity of mTOR for compounds having related structures to rapamycin, but decreases the affinity of mTOR for rapamycin itself. Thus, inclusion of a threonine at this position maintains the ability of mTOR to bind to rapamycin. The amino acid of a CISC component or FRB domain that “corresponds to” amino acid 2098 of wild-type mTOR may be determined by aligning a candidate sequence of a CISC component or FRB domain to SEQ ID NO: 236 (e.g., by BLAST or another alignment algorithm known in the art), with the amino acid aligned to amino acid 2098 of SEQ ID NO: 236 being the amino acid that “corresponds to” amino acid 2098 of SEQ ID NO: 236.

Each of the extracellular binding domains, transmembrane domains, and intracellular signaling domains of the CISC components described herein may be connected to another domain of the same CISC component by a linker. Linkers are known in the art. In some embodiments, the linker comprises 1, 2, 3, 4, 5, 6, 7, 8, 9 or 10 amino acids, such as glycines, or a number of amino acids, such as glycine, within a range defined by any two of the aforementioned numbers. In some embodiments, the glycine spacer comprises at least 3 glycines. In some embodiments, the glycine spacer comprises a sequence set forth as GSG, GGGS (SEQ ID NO: 229), GGGSGGG (SEQ ID NO: 230) or GGG. In some embodiments, the glycine spacer comprises the amino acid sequence GSG.

An extracellular binding domain may be connected to a transmembrane domain by a hinge domain. A hinge refers to a domain that links the extracellular binding domain to the transmembrane domain, and may confer flexibility to the extracellular binding domain. In some embodiments, the hinge domain positions the extracellular binding domain close to the plasma membrane to minimize the potential for recognition by antibodies or binding fragments thereof. In some embodiments, the extracellular binding domain is located N-terminal to the hinge domain. In some embodiments, the hinge domain may be natural or synthetic.

In some embodiments, the first and second CISC components form a heterodimer in the presence of rapamycin. In some embodiments, the first and second CISC components form a heterodimer in the presence of a compound that produced in vivo by metabolism of a rapalog. In some embodiments, the compound produced by in vivo metabolism of the rapalog is rapamycin. Non-limiting examples of rapalogs include everolimus, CCI-779, C20-methallylrapamycin, C16-(S)-3-methylindolerapamycin, C16-iRap, C16-(S)-7-methylindolerapamycin, AP21967, C16-(S)Butylsulfonamidorapamycin, AP23050, sodium mycophenolic acid, benidipine hydrochloride, AP1903, and AP23573, and metabolites or derivatives thereof.

In some embodiments, the nucleic acid encoding the second CISC component (FRB-IL2Rβ) further comprises a nucleotide sequence encoding a third CISC component that is capable of binding to rapamycin. Such CISC components are useful, for example, for binding to intracellular rapamycin, thereby preventing the bound rapamycin from interacting with other intracellular molecules or structures (e.g., preventing rapamycin from interacting with mTOR). In some embodiments, the third CISC component is a soluble protein that does not comprise a transmembrane domain. In some embodiments, the third CISC component comprises an intracellular FRB domain. In some embodiments, a third CISC component is a soluble protein comprising an FRB domain and lacking a transmembrane domain.

Nucleic acids encoding a first, second, and/or third CISC component may be comprised in one or more vectors. In some embodiments, a nucleic acid encoding a first CISC component is present on a separate vector from a nucleic acid encoding the second CISC component. In some embodiments, a nucleic acid encoding the third CISC component is present on the same vector as a nucleic acid encoding the second CISC component. In some embodiments, one or more vectors are viral vectors. In some embodiments, one or more vectors are adeno-associated viral (AAV) vectors. In some embodiments, one or more AAV vectors is an AAV1, AAV2, AAV3, AAV4, AAV5, AAV6, AAV7, AAV8, AAV9, AAV10, or AAV11 vector. In some embodiments, one or more AAV vectors are AAV5 vectors. In some embodiments, one or more AAV vectors are AAV6 vectors.

In some embodiments, a CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 66 or 71. In some embodiments, one or more CISC components further comprise a signal peptide. The signal peptide may be any signal peptide known in the art that directs the translated CISC component to the cell membrane. In some embodiments, each of the first and second CISC components comprises an LCN2 signal peptide. In some embodiments, each of the first and second CISC components comprises a signal peptide comprising the amino acid sequence of SEQ ID NO: 61

In some embodiments, one CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 66, and the other CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 71. In some embodiments, each CISC component further comprises a signal peptide, which may have the same or different amino acid sequences. The signal peptides may be any signal peptide known in the art that directs the translated CISC component to the cell membrane.

In some embodiments, a third CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 72. In some embodiments, a third CISC component consists of an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 72. In some embodiments, the third CISC component comprises the amino acid sequence of SEQ ID NO: 72. In some embodiments, the third CISC component consists of the amino acid sequence of SEQ ID NO: 72. In some embodiments, the third CISC component does not comprise a signal peptide. In some embodiments, the third CISC component does not comprise a transmembrane domain.

### T Cell Receptors (TCRs)

In some embodiments of the methods described herein, the TRAC locus of a cell is edited by inserting a nucleic acid comprising a promoter operably linked to a nucleotide sequence encoding a full-length TCRβ protein, and to a nucleotide sequence encoding at least a portion of a TCRα protein, such as TCRα variable and TCRα joining (TRAJ) regions that form the portion of a TCRα protein responsible for antigen-specificity. In some embodiments the nucleotide sequence encoding the TCRα variable and joining regions inserted in-frame with the endogenous nucleotide sequence encoding a portion of the TCRα constant domain, such that the inserted heterologous promoter initiates transcription of a sequence encoding a heterologous TCRβ protein and a sequence encoding a TCRα protein comprising heterologous TRAV/TRAJ amino acid sequences and an endogenous TCRα constant domain. This embodiment utilizes the endogenous 3′ regulatory region from the endogenous TRAC locus.

Genetically modified cells produced by methods described herein express a T cell receptor specific to a type 1 diabetes (T1D)-associated antigen. As used herein, a “T cell receptor” (TCR) refers to an immunoglobulin superfamily member having a variable binding domain, a constant domain, a transmembrane region, and a short cytoplasmic tail. See, e.g., Janeway et al., Immunobiology: The Immune System in Health and Disease, 3rd Ed., Current Biology Publications, p. 433, 1997. A TCR is capable of specifically binding to an antigen peptide bound to a major histocompatibility complex encoded (MHC) receptor. A TCR can be found on the surface of a T cell or may be released into the extracellular milieu in soluble form, and generally is comprised of a heterodimer having α and β chains (also known as TCR α and TCRβ, respectively), or γ and δ chains (also known as TCRγ and TCRδ, respectively), each having a constant (C) domain, and a and highly polymorphic variable (V) domain, each variable domain comprising three complementarity determining regions (CDR) that are largely responsible for specific antigen recognition and binding by the TCR. In certain embodiments, a nucleic acid encoding a TCR can be codon-optimized to enhance expression in a particular host cell, such as, for example, a cell of the immune system, a hematopoietic stem cell, a T cell, a primary T cell, a T cell line, a NK cell, or a natural killer T cell. See, e.g., Scholten et al., Clin Immunol. 2006. 119:135.

Like other antigen-binding members of the immunoglobulin superfamily (e.g., antibodies), the extracellular domains of TCR chains (e.g., TCRα chain and TCRβ) contain two immunoglobulin domains, a variable domain (e.g., α-chain variable domain or Vα, β-chain variable domain or Vβ; typically amino acids 1 to 116 based on Kabat numbering (Kabat et al., “Sequences of Proteins of Immunological Interest, US Dept. Health and Human Services, Public Health Service National Institutes of Health, 1991, 5th ed.)) at the N-terminus, and one constant domain (e.g., α-chain constant domain or Cα, typically 5 amino acids 117 to 259 based on Kabat, β-chain constant domain or Cβ, typically amino acids 117 to 295 based on Kabat) adjacent the cell membrane. Also, like immunoglobulins, the variable domains contain complementary determining regions (CDRs) separated by framework regions (FRs) (see, e.g., Jores et al., Proc. Nat'l Acad. Sci. USA 87:9138, 1990; Chothia et al., EMBO J. 7:3745, 1988; see also Lefranc et al., Dev. Comp. Immunol. 27:55, 2003). The source of a TCR as used in the present disclosure may be from various animal species, such as a human, non-human primate, mouse, rat, rabbit, or other mammal.

The term “variable region” or “variable domain” refers to the structural domain of an immunoglobulin superfamily binding protein (e.g., a TCR α-chain or β-chain (or γ chain and δ chain for γδ TCRs)) that is involved in specific binding of the immunoglobulin superfamily binding protein (e.g., TCR) to antigen. The variable domains of the α chain and β chain (Vα and Vβ, respectively) of a native TCR generally have similar structures, with each domain comprising four generally conserved framework regions (FRs) and three CDRs. The Vα domain is encoded by two separate DNA segments, the variable gene segment and the joining gene segment (V-J); the Vβ domain is encoded by three separate DNA segments, the variable gene segment, the diversity gene segment, and the joining gene segment (V-D-J). A single Vα or Vβ domain may be sufficient to confer antigen-binding specificity. Furthermore, TCRs that bind a particular antigen may be isolated using a Vα or Vβ domain from a TCR that binds the antigen to screen a library of complementary Vα or Vβ domains, respectively.

The terms “complementarity determining region,” and “CDR,” are synonymous with “hypervariable region” or “HVR,” and are known in the art to refer to sequences of amino acids within immunoglobulin (e.g., TCR) variable regions, which confer antigen specificity and/or binding affinity and are separated from one another in primary amino acid sequence by framework regions. In general, there are three CDRs in each TCR α-chain variable region (αCDR1, αCDR2, αCDR3) and three CDRs in each TCR β-chain variable region (βCDR1, βCDR2, βCDR3). In TCRs, CDR3 is thought to be the main CDR responsible for recognizing a peptide antigen bound to MHC. In general, CDR1 and CDR2 interact mainly or exclusively with the MHC.

CDR1 and CDR2 are encoded within the variable gene segment of a TCR variable domain coding sequence, whereas CDR3 is encoded by the region spanning the variable and joining segments for Vα, or the region spanning variable, diversity, and joining segments for Vβ. Thus, if the identity of the variable gene segment of a Vα or Vβ is known, the sequences of their corresponding CDR1 and CDR2 can be deduced; e.g., according to a numbering scheme as described herein. Compared with CDR1 and CDR2, CDR3 is typically significantly more diverse due to the addition and loss of nucleotides during the recombination process.

TCR variable domain sequences can be aligned to a numbering scheme (e.g., Kabat, Chothia, EU, IMGT, Enhanced Chothia, and Aho), allowing equivalent residue positions to be annotated and for different molecules to be compared using, for example, ANARCI software tool (2016, Bioinformatics 15:298-300). A numbering scheme provides a standardized delineation of framework regions and CDRs in the TCR variable domains. In certain embodiments, a CDR of the present disclosure is identified according to the IMGT numbering scheme (Lefranc et al., Dev. Comp. Immunol. 27:55, 2003; imgt.org/IMGTindex/V-QUEST.php).

In some embodiments, a nucleic acid described herein encodes a TCRβ chain and at least a portion of a TCRα chain that, expressed in combination, form a T1D2 TCR that binds to a peptide of IGRP(305-234). In other embodiments, a TCRβ chain and full-length TCRα chain, a portion of which is encoded by a nucleic acid described herein, form a T1D4 TCR that binds a peptide of IGRP(241-260). In other embodiments, a TCRβ chain and full-length TCRα chain, a portion of which is encoded by a nucleic acid described herein, form a T1D5-1 TCR that binds a peptide of IGRP(305-324). In some embodiments, the peptide of IGRP(305-324) is recognized when bound to HLA-DRB1*0401. In some embodiments, the peptide of IGRP(241-260) is recognized when bound to HLA-DRB1*0401.

In some embodiments, a TCR formed by a TCRβ chain and (at least a portion of) the TCRα chain encoded by a nucleic acid described herein comprises a TCRα variable (Vα) domain having three complementarity determining regions (CDRs) of αCDR1, αCDR2, and αCDR3; and a TCRβ variable (Vβ) domain having three CDRs of βCDR1, βCDR2, and βCDR3. Representative amino acids of CDRs of TCRs described herein are shown in Table 1, and nucleotide sequences encoding the same are shown in Table 2. In some embodiments: (i) αCDR1 comprises SEQ ID NO: 1, (ii) αCDR2 comprises SEQ ID NO: 2, (iii) αCDR3 comprises SEQ ID NO: 3, (iv) βCDR1 comprises SEQ ID NO: 4, (v) βCDR2 comprises SEQ ID NO: 5, and (vi) βCDR3 comprises SEQ ID NO: 6. In some embodiments: (i) αCDR1 comprises SEQ ID NO: 11, (ii) αCDR2 comprises SEQ ID NO: 12, (iii) αCDR3 comprises SEQ ID NO: 13, (iv) βCDR1 comprises SEQ ID NO: 14, (v) βCDR2 comprises SEQ ID NO: 15, and (vi) βCDR3 comprises SEQ ID NO: 16. In some embodiments: (i) αCDR1 comprises SEQ ID NO: 21, (ii) αCDR2 comprises SEQ ID NO: 22, (iii) αCDR3 comprises SEQ ID NO: 23, (iv) βCDR1 comprises SEQ ID NO: 24, (v) βCDR2 comprises SEQ ID NO: 25, and (vi) βCDR3 comprises SEQ ID NO: 26. In other embodiments, each of the set of αCDR1, αCDR2, αCDR3, βCDR1, βCDR2, and βCDR3 may have an amino acid sequence having at least 80%, at least 85%, at least 90%, at least 91%, at least 92%, at least 93%, at least 94%, at least 95%, at least 96%, at least 97%, at least 98%, or at least 99% sequence identity to the respective amino acid sequences in any of the aforementioned combinations of amino acid sequences.

In some embodiments, Vα comprises SEQ ID NO: 7 and Vβ comprises SEQ ID NO: 8. In some embodiments, Vα comprises SEQ ID NO: 17 and Vβ comprises SEQ ID NO: 18. In some embodiments, Vα comprises SEQ ID NO: 27 and Vβ comprises SEQ ID NO: 28. In other embodiments, each of the pair of Vα and Vβ may have an amino acid sequence having at least 80%, at least 85%, at least 90%, at least 91%, at least 92%, at least 93%, at least 94%, at least 95%, at least 96%, at least 97%, at least 98%, or at least 99% sequence identity to the respective amino acid sequence any of the aforementioned combinations of amino acid sequences.

In some embodiments, the TCRα chain comprises SEQ ID NO: 9 and the TCRβ chain comprises SEQ ID NO: 10. In some embodiments, the TCRα chain comprises SEQ ID NO: 19 and the TCRβ chain comprises SEQ ID NO: 20. In some embodiments, the TCRα chain comprises SEQ ID NO: 29 and the TCRβ chain comprises SEQ ID NO: 30. In other embodiments, each of the pair of TCRα and TCRβ chains may have an amino acid sequence having at least 80%, at least 85%, at least 90%, at least 91%, at least 92%, at least 93%, at least 94%, at least 95%, at least 96%, at least 97%, at least 98%, or at least 99% sequence identity to the respective amino acid sequence of any of the aforementioned combinations of amino acid sequences.

### FOXP3 Locus Modification

In some embodiments of the methods described herein, the FOXP3 locus of a cell is edited by inserted a nucleic acid comprising a promoter operably linked to a nucleotide sequence encoding a portion of the endogenous FoxP3 protein. The inserted promoter is introduced into the genome downstream from the Treg-specific demethylated region (TSDR) of the FOXP3 locus. In unmodified cells, the TSDR epigenetically regulates expression of FoxP3, inhibiting FoxP3 production in cells exposed to inflammatory conditions, which may result in loss of FoxP3 expression and conversion of unmodified Treg cells to a T effector (Teff) phenotype. Insertion of a promoter downstream from the TSDR bypasses TSDR-mediated regulation of FOXP3 expression, thereby providing stable production of FoxP3 even in inflammatory conditions.

The heterologous promoter may be inserted at any position downstream from the endogenous promoter (e.g., downstream from the TSDR) and upstream from or within the first coding exon of the FOXP3 coding sequence. This first coding exon is known in the art as exon 2, as it is the second exon present in pre-mRNA transcribed from the endogenous FOXP3 promoter, and the first coding exon because it is this exon, not exon 1 (the first exon of FOXP3-encoding pre-mRNA) that contains the start codon that initiates translation of wild-type FoxP3. In some embodiments, the heterologous promoter is inserted 1-10,000, 10-1,000, 10-100, 10-5,000, 20-4,000, 30-3,000, 40-2,000, 50-1,000, 60-750, 70-500, 80-400, 90-300, 100-200, 1-1,000, 1,000-2,000, 2,000-3,000, 3,000-4,000, 4,000-5,000, 5,000-6,000, 6,000-7,000, 7,000-8,000, 8,000-9,000, or 9,000-10,000 nucleotides downstream from the TSDR of FOXP3. In some embodiments, the heterologous promoter is inserted 1-10,000, 10-1,000, 10-100, 10-5,000, 20-4,000, 30-3,000, 40-2,000, 50-1,000, 60-750, 70-500, 80-400, 90-300, 100-200, 1-1,000, 1,000-2,000, 2,000-3,000, 3,000-4,000, 4,000-5,000, 5,000-6,000, 6,000-7,000, 7,000-8,000, 8,000-9,000, or 9,000-10,000 nucleotides upstream from the first coding exon of the FOXP3 coding sequence. In some embodiments, the heterologous promoter is inserted into the first coding exon, such that a synthetic first coding exon is created, where the synthetic first coding exon differs from the endogenous first coding exon but still comprises a start codon that is in-frame with the FOXP3 coding sequence of downstream FOXP3 exons.

### 2A Motifs and Linkers

Some embodiments of nucleic acids described herein encoding multiple polypeptides or portions thereof may contain intervening nucleotide sequences encoding a 2A motifs. 2A motifs are known in the art, and are useful for promoting production of multiple polypeptides from translation of a single nucleotide sequence. See, e.g., Kim et al., PLoS ONE. 2011. 6:e18556. In some embodiments, the 2A motif is translated, and self-cleavage of the polypeptide occurs following translation, resulting in release of separate polypeptides. In other embodiments, the nucleotide sequence encoding the 2A motif causes the ribosome to progress along an mRNA without incorporating an encoded amino acid of the 2A motif, resulting in release of the first polypeptide (e.g., first FKBP-IL2Rγ CISC component), and allowing translation initiation of a second polypeptide (e.g., TCRβ chain).

In some embodiments, nucleotide sequences encoding a 2A motif are present in-frame with and between each pair of nucleotide sequences encoding (i) the first (FKBP-IL2Rγ) CISC component; (ii) the TCRβ chain; and (iii) the TCRα chain or portion thereof. Thus, the heterologous promoter (e.g., MND promoter) initiates transcription of a single mRNA encoding each of the CISC component, TCRβ chain, and TCRα chain, with intervening 2A motifs allowing production of each as a separate polypeptide. In some embodiments, a nucleotide sequence encoding a 2A motif is in-frame with and between each pair of nucleotide sequences encoding (i) the second (FKBP-IL2Rγ) CISC component; (ii) the cytosolic FRB domain; and (iii) FoxP3. Thus, the heterologous promoter (e.g., MND promoter) initiates transcription of a single mRNA encoding each of the CISC component, cytosolic FRB domain, and FoxP3, with intervening 2A motifs allowing production of each as a separate polypeptide.

The 2A motifs encoded by nucleotide sequences between each pair of sequences encoding two polypeptides (e.g., sequences encoding an FKBP-IL2Rγ CISC component and TCRβ chain; TCRβ chain and portion of α chain) may be any 2A motif known in the art. In some embodiments, the encoded 2A motifs between each pair of nucleotide sequences encoding distinct polypeptides may be independently selected from the group consisting of F2A, P2A, T2A, E2A. In some embodiments, a first encoded 2A motif and second encoded 2A motif on a nucleic acid are different 2A motifs. Use of different 2A motifs in the same inserted nucleic acid reduces the probability of internal recombination, which may result in the nucleotide sequence between the recombined 2A motifs being excised from the chromosome. In some embodiments, a nucleotide sequence encoding a first 2A motif has no more than 90% sequence identity to a nucleotide sequence encoding a second 2A motif on the same nucleic acid. In some embodiments, a nucleotide sequence encoding a first 2A motif has no more than 80% sequence identity to a nucleotide sequence encoding a second 2A motif on the same nucleic acid. In some embodiments, a nucleotide sequence encoding a first 2A motif has no more than 70% sequence identity to a nucleotide sequence encoding a second 2A motif on the same nucleic acid. In some embodiments, a nucleotide sequence encoding a first 2A motif has no more than 60% sequence identity to a nucleotide sequence encoding a second 2A motif on the same nucleic acid. In some embodiments, a nucleotide sequence encoding a first 2A motif has no more than 50% sequence identity to a nucleotide sequence encoding a second 2A motif on the same nucleic acid. In some embodiments, a first 2A motif is a T2A motif, and the second motif is a P2A motif.

In other embodiments, the first and second 2A motifs encoded by nucleotide sequences on the nucleic acid are the same 2A motif. In some embodiments, a nucleic acid comprises a nucleotide sequence encoding a first P2A motif, and a second nucleotide sequence encoding a second P2A motif, with the nucleotide sequence encoding the first P2A motif comprising at least 80% sequence identity to the nucleotide sequence encoding the second P2A motif. In some embodiments, the first and second nucleotide sequences encoding the first and second P2A motifs comprise the same nucleotide sequences.

In some embodiments, the nucleic acid for insertion into the TRAC locus comprises: (i) a sequence encoding a T2A motif between the sequence encoding the first CISC component and the sequence encoding the TCRβ chain; and (ii) a sequence encoding a P2A motif between the sequence encoding the TCRβ chain and heterologous TCRα chain portion.

In some embodiments, the nucleic acid for insertion into the FOXP3 locus comprises: (i) a sequence encoding a P2A motif between the sequence encoding the second CISC component and the sequence encoding the cytosolic FRB domain; and (ii) a second sequence encoding a second P2A motif between the sequence encoding the cytosolic FRB domain and the sequence encoding FoxP3.

In some embodiments, a polypeptide (e.g., CISC components and/or TCRβ chains) encoded by a nucleic acid for insertion into the cell genome comprises a C-terminal linker. Incorporation of such a linker may, for example, improve efficiency of cleavage in 2A motifs and/or prevent cleavage of a 2A motif from excising amino acids of the encoded CISC component or TCRβ chain. In some embodiments, the encoded first CISC component comprises a C-terminal linker. In some embodiments, the encoded second CISC component comprises a C-terminal linker. In some embodiments, the encoded cytosolic FRB domain component comprises a C-terminal linker. In some embodiments, the encoded TCRβ chain comprises a C-terminal linker.

Linkers at the C-terminus of encoded polypeptides may be any linker known in the art. In some embodiments, the linker comprises 1, 2, 3, 4, 5, 6, 7, 8, 9 or 10 amino acids, such as glycines, or a number of amino acids, such as glycine, within a range defined by any two of the aforementioned numbers. In some embodiments, the linker comprises at least 3 glycines. In some embodiments, the linker comprises a sequence set forth as GSG, GGGS (SEQ ID NO: 229), GGGSGGG (SEQ ID NO: 230) or GGG. In some embodiments, the linker comprises the amino acid sequence GSG. In some embodiments, each of the first CISC component, second CISC component, cytosolic FRB domain, and TCRβ chain comprises a C-terminal linker having the amino acid sequence GSG.

### Vectors

The first and/or second nucleic acids for insertion into the TRAC and FOXP3 loci, respectively, may be comprised in one or more vectors. In some embodiments, the first TRAC locus-targeting nucleic acid is comprised in a first vector, and the FOXP3 locus-targeting nucleic acid is comprised in a second vector. In some cases, the vector is packaged in a virus capable of infecting the cell (e.g., the vector is a viral vector). Exemplary viruses include adenovirus, retrovirus, lentivirus, adeno-associated virus, and others that are known in the art and disclosed herein.

The term “vector” is used to refer to any molecule (e.g., nucleic acid, plasmid) or arrangement of molecules (e.g., virus) used to transfer coding information to a host cell. The term “expression vector” refers to a vector that is suitable for introduction of a host cell and contains nucleic acid sequences that direct and/or control expression of introduced heterologous nucleic acid sequences. Expression includes, but is not limited to, processes such as transcription, translation, and RNA splicing, if introns are present. Non-limiting examples of vectors include artificial chromosomes, minigenes, cosmids, plasmids, phagemids, and viral vectors. Non-limiting examples of viral vectors include lentiviral vectors, retroviral vectors, herpesvirus vectors, adenovirus vectors, and adeno-associated viral vectors. In some embodiments, one or more vectors comprising nucleic acids for use in the methods provided herein are lentiviral vectors. In some embodiments, one or more vectors are adenoviral vectors. In some embodiments, one or more vectors are adeno-associated viral (AAV) vectors. In some embodiments, one or more AAV vectors is an AAV1, AAV2, AAV3, AAV4, AAV5, AAV6, AAV7, AAV8, AAV9, AAV10, or AAV 11 vector. In some embodiments, a vector comprising the nucleic acid for insertion into the TRAC locus is an AAV1, AAV2, AAV3, AAV4, AAV5, AAV6, AAV7, AAV8, AAV9, AAV10, or AAV11 vector. In some embodiments, a vector comprising the nucleic acid for insertion into the FOXP3 locus is an AAV1, AAV2, AAV3, AAV4, AAV5, AAV6, AAV7, AAV8, AAV9, AAV10, or AAV11 vector.

In some embodiments, one or more AAV vectors are AAV5 vectors. In some embodiments, one or more AAV vectors are AAV6 vectors. In some embodiments, both the first and second nucleic acids are comprised in separate AAV5 vectors. In some embodiments, both the first and second nucleic acids are comprised in separate AAV6 vectors.

In some embodiments, a nucleic acid for insertion into the TRAC locus comprises, between the 5′ and 3′ homology arms, a nucleotide sequence having at least 90% sequence identity to any one of SEQ ID NOs: 94, 106, 117, 128, and 139. In some embodiments, the nucleotide sequence comprises at least 95% sequence identity to any one of SEQ ID NOS: 94, 106, 117, 128, and 139. In some embodiments, the nucleotide sequence comprises any one of SEQ ID NOS: 94, 106, 117, 128, and 139. In some embodiments, the nucleic acid comprises the nucleotide sequence of SEQ ID NO: 94. In some embodiments, the nucleic acid comprises the nucleotide sequence of SEQ ID NO: 106. In some embodiments, the nucleic acid comprises the nucleotide sequence of SEQ ID NO: 117. In some embodiments, the nucleic acid comprises the nucleotide sequence of SEQ ID NO: 128. In some embodiments, the nucleic acid comprises the nucleotide sequence of SEQ ID NO: 139.

In some embodiments, a nucleic acid for insertion into the TRAC locus comprises at least 90% sequence identity to the nucleotide sequence of any one of SEQ ID NOs: 95, 107, 118, 129, and 140. In some embodiments, the nucleic acid comprises at least 95% sequence identity to the nucleotide sequence of any one of SEQ ID NOs: 95, 107, 118, 129, and 140. In some embodiments, the nucleic acid comprises the nucleotide sequence of any one of SEQ ID NOs: 95, 107, 118, 129, and 140. In some embodiments, the nucleic acid comprises at least 95% sequence identity to the nucleotide sequence of SEQ ID NO: 95. In some embodiments, the nucleic acid comprises at least 95% sequence identity to the nucleotide sequence of SEQ ID NO: 107. In some embodiments, the nucleic acid comprises at least 95% sequence identity to the nucleotide sequence of SEQ ID NO: 118. In some embodiments, the nucleic acid comprises at least 95% sequence identity to the nucleotide sequence of SEQ ID NO: 129. In some embodiments, the nucleic acid comprises at least 95% sequence identity to the nucleotide sequence of SEQ ID NO: 140.

In some embodiments, the nucleic acid comprises the nucleotide sequence of SEQ ID NO: 95. In some embodiments, the nucleic acid comprises the nucleotide sequence of SEQ ID NO: 107. In some embodiments, the nucleic acid comprises the nucleotide sequence of SEQ ID NO: 118. In some embodiments, the nucleic acid comprises the nucleotide sequence of SEQ ID NO: 129. In some embodiments, the nucleic acid comprises the nucleotide sequence of SEQ ID NO: 140.

In some embodiments, a nucleic acid for insertion into the FOXP3 locus comprises, between the 5′ and 3′ homology arms, a nucleotide sequence having at least 90% sequence identity to any one of SEQ ID NOs: 150, 161, 172, 184, 195, 206, and 218. In some embodiments, the nucleotide sequence comprises at least 95% sequence identity to any one of SEQ ID NOS: 150, 161, 172, 184, 195, 206, and 218. In some embodiments, the nucleotide sequence comprises any one of SEQ ID NOS: 150, 161, 172, 184, 195, 206, and 218. In some embodiments, the nucleic acid comprises the nucleotide sequence of SEQ ID NO: 150. In some embodiments, the nucleic acid comprises the nucleotide sequence of SEQ ID NO: 161. In some embodiments, the nucleic acid comprises the nucleotide sequence of SEQ ID NO: 172. In some embodiments, the nucleic acid comprises the nucleotide sequence of SEQ ID NO: 184. In some embodiments, the nucleic acid comprises the nucleotide sequence of SEQ ID NO: 195. In some embodiments, the nucleic acid comprises the nucleotide sequence of SEQ ID NO: 206. In some embodiments, the nucleic acid comprises the nucleotide sequence of SEQ ID NO: 218.

In some embodiments, a nucleic acid for insertion into the FOXP3 locus comprises at least 90% sequence identity to the nucleotide sequence of any one of SEQ ID NOs: 151, 162, 173, 185, 196, 207, and 219. In some embodiments, the nucleic acid comprises at least 95% sequence identity to the nucleotide sequence of any one of SEQ ID NOs: 151, 162, 173, 185, 196, 207, and 219. In some embodiments, the nucleic acid comprises the nucleotide sequence of any one of SEQ ID NOs: 151, 162, 173, 185, 196, 207, and 219. In some embodiments, the nucleic acid comprises at least 95% sequence identity to the nucleotide sequence of SEQ ID NO: 151. In some embodiments, the nucleic acid comprises at least 95% sequence identity to the nucleotide sequence of SEQ ID NO: 162. In some embodiments, the nucleic acid comprises at least 95% sequence identity to the nucleotide sequence of SEQ ID NO: 173. In some embodiments, the nucleic acid comprises at least 95% sequence identity to the nucleotide sequence of SEQ ID NO: 185. In some embodiments, the nucleic acid comprises at least 95% sequence identity to the nucleotide sequence of SEQ ID NO: 196. In some embodiments, the nucleic acid comprises at least 95% sequence identity to the nucleotide sequence of SEQ ID NO: 207. In some embodiments, the nucleic acid comprises at least 95% sequence identity to the nucleotide sequence of SEQ ID NO: 219.

In some embodiments, the nucleic acid comprises the nucleotide sequence of SEQ ID NO: 151. In some embodiments, the nucleic acid comprises the nucleotide sequence of SEQ ID NO: 162. In some embodiments, the nucleic acid comprises the nucleotide sequence of SEQ ID NO: 173. In some embodiments, the nucleic acid comprises the nucleotide sequence of SEQ ID NO: 185. In some embodiments, the nucleic acid comprises the nucleotide sequence of SEQ ID NO: 196. In some embodiments, the nucleic acid comprises the nucleotide sequence of SEQ ID NO: 207. In some embodiments, the nucleic acid comprises the nucleotide sequence of SEQ ID NO: 219.

### Homology Arms

Nucleic acids for insertion into TRAC or FOXP3 loci in the methods described herein comprise 5′ and 3′ homology arms, to target insertion of the nucleic acid into the TRAC or FOXP3 locus, respectively, by homology-directed repair following introduction of a double-stranded break. Typically, the 5′ homology arm refers to a homology arm at the 5′ end of the nucleic acid, and 3′ homology arm refers to another homology arm at the 3′ end of the nucleic acid, when considering the coding strand of the nucleic acid (i.e., the strand containing the reading frame(s) encoding polypeptides including CISC components, TCR chains, and FoxP3). The 5′ homology arm will have homology to a first sequence in the targeted locus, and the 3′ homology arm will have homology to a second sequence in the targeted locus that is downstream from the first sequence in the targeted locus, such that the nucleic acid is inserted into the locus in a targeted manner. Following insertion, the modified locus will comprise the homology arms, in place of the first and second sequences in the targeted locus, and the sequence between the homology arms on the nucleic acid, in place of the sequence that was previously present between the first and second sequences in the targeted locus. The homology arms may be the same length, have similar lengths (within 100 bp of each other), or different lengths. In some embodiments, one or both homology arms have a length of 100-2,000 bp, 200-2,000 bp, 400-1,500 bp, 500-1,000 bp. In some embodiments, one or both homology arms are about 100 bp, about 200 bp, about 300 bp, about 400 bp, about 500 bp, about 600 bp, about 700 bp, about 800 bp, about 900 bp, about 1,000 bp, about 1,100 bp, about 1,200 bp, about 1,300 bp, about 1,400 bp, about 1,500 bp, about 1,600 bp, about 1,700 bp, about 1,800 bp, about 1,900 bp, or about 2,000 bp. In some embodiments, both homology arms are 100-2,000 nucleotides in length. In some embodiments, both homology arms are 300-1,000 nucleotides in length. In some embodiments, both homology arms are 300-700 nucleotides in length. In some embodiments, both homology arms are 300-500 nucleotides in length. In some embodiments, both homology arms are 500-700 nucleotides in length. In some embodiments, both homology arms are 700-1,000 nucleotides in length.

Homology arms of a nucleic acid for insertion at a targeted genomic locus may be chosen based on homologous sequences in the targeted locus that are upstream and/or downstream from a site targeted for cleavage by a nuclease. For example, in some embodiments for insertion by homology-directed repair following cleavage at a given position (cleavage site) in the targeted locus, the 5′ homology arm of a nucleic acid for insertion has homology to a sequence upstream of the cleavage site, and the 3′ homology arm of the nucleic acid has homology to a sequence downstream of the cleavage site. In some embodiments, the 5′ homology arm has homology to a sequence 100-2,000 nucleotides in length that ends 25-5,000, 50-3,000, 75-2,000, 100-1,000, 150-500 nucleotides upstream from the cleavage site. In some embodiments, the 5′ homology arm has homology to a sequence 100-2,000 nucleotides in length that ends 25-5,000, 50-3,000, 75-2,000, 100-1,000, 150-500 nucleotides upstream from a PAM sequence cleaved by an RNA-guided nuclease. In some embodiments, the 5′ homology arm has homology to a sequence 100-2,000 nucleotides in length that ends 25-5,000, 50-3,000, 75-2,000, 100-1,000, 150-500 nucleotides upstream from a sequence in the genome that is complementary to a spacer sequence of a gRNA. In some embodiments, the 5′ homology arm has homology to a sequence 100-2,000 nucleotides in length that ends at a position 150-500 nucleotides upstream from a cleavage site. In some embodiments, the 5′ homology arm has homology to a sequence 100-2,000 nucleotides in length that ends at a position 150-500 nucleotides upstream from a PAM sequence cleaved by an RNA-guided nuclease. In some embodiments, the 5′ homology arm has homology to a sequence 100-2,000 nucleotides in length that ends at a position 150-500 nucleotides upstream from a sequence in the genome that is complementary to a spacer sequence of a gRNA.

In some embodiments, the 3′ homology arm has homology to a sequence 100-2,000 nucleotides in length that ends 25-5,000, 50-3,000, 75-2,000, 100-1,000, 150-500 nucleotides upstream from the cleavage site. In some embodiments, the 3′ homology arm has homology to a sequence 100-2,000 nucleotides in length that ends 25-5,000, 50-3,000, 75-2,000, 100-1,000, 150-500 nucleotides upstream from a PAM sequence cleaved by an RNA-guided nuclease. In some embodiments, the 3′ homology arm has homology to a sequence 100-2,000 nucleotides in length that ends 25-5,000, 50-3,000, 75-2,000, 100-1,000, 150-500 nucleotides upstream from a sequence in the genome that is complementary to a spacer sequence of a gRNA. In some embodiments, the 3′ homology arm has homology to a sequence 100-2,000 nucleotides in length that ends at a position 150-500 nucleotides upstream from a cleavage site. In some embodiments, the 3′ homology arm has homology to a sequence 100-2,000 nucleotides in length that ends at a position 150-500 nucleotides upstream from a PAM sequence cleaved by an RNA-guided nuclease. In some embodiments, the 3′ homology arm has homology to a sequence 100-2,000 nucleotides in length that ends at a position 150-500 nucleotides upstream from a sequence in the genome that is complementary to a spacer sequence of a gRNA.

In some embodiments, where a method includes a gRNA comprising a spacer sequence, neither the 5′ nor the 3′ homology arm of a nucleic acid for genomic insertion comprises a sequence that is complementary to the spacer sequence. In such embodiments, lack of a complementary sequence on the donor template reduces the chance of the gRNA binding to the donor template and mediating cleavage, which can reduce the efficiency of genomic insertion. In some embodiments, the donor template does not comprise a sequence that is complementary to the spacer sequence. In embodiments where a different nuclease that does not require a gRNA for targeted cleavage is used, the donor template does not comprise a sequence that is cleaved by the nuclease.

In some embodiments, a nucleic acid for insertion into the TRAC locus comprises a 5′ homology arm with at least 90% sequence identity to the nucleotide sequence of SEQ ID NO: 85, and a 3′ homology arm with at least 90% sequence identity to the nucleotide sequence of SEQ ID NO: 93. In some embodiments, the 5′ homology arm comprises at least 95% sequence identity to the nucleotide sequence of SEQ ID NO: 85, and the 3′ homology arm comprises at least 95% to the nucleotide sequence of SEQ ID NO: 93. In some embodiments, the 5′ homology arm comprises the nucleotide sequence of SEQ ID NO: 85, and the 3′ homology arm comprises the nucleotide sequence of SEQ ID NO: 93.

In some embodiments, a nucleic acid for insertion into the TRAC locus comprises a 5′ homology arm with at least 90% sequence identity to the nucleotide sequence of SEQ ID NO: 96, and a 3′ homology arm with at least 90% sequence identity to the nucleotide sequence of SEQ ID NO: 105. In some embodiments, the 5′ homology arm comprises at least 95% sequence identity to the nucleotide sequence of SEQ ID NO: 96, and the 3′ homology arm comprises at least 95% to the nucleotide sequence of SEQ ID NO: 105. In some embodiments, the 5′ homology arm comprises the nucleotide sequence of SEQ ID NO: 96, and the 3′ homology arm comprises the nucleotide sequence of SEQ ID NO: 105.

In some embodiments, a nucleic acid for insertion into the TRAC locus comprises a 5′ homology arm with at least 90% sequence identity to the nucleotide sequence of SEQ ID NO: 108, and a 3′ homology arm with at least 90% sequence identity to the nucleotide sequence of SEQ ID NO: 116. In some embodiments, the 5′ homology arm comprises at least 95% sequence identity to the nucleotide sequence of SEQ ID NO: 108, and the 3′ homology arm comprises at least 95% to the nucleotide sequence of SEQ ID NO: 116. In some embodiments, the 5′ homology arm comprises the nucleotide sequence of SEQ ID NO: 108, and the 3′ homology arm comprises the nucleotide sequence of SEQ ID NO: 116.

In some embodiments, a nucleic acid for insertion into the TRAC locus comprises a 5′ homology arm with at least 90% sequence identity to the nucleotide sequence of SEQ ID NO: 119, and a 3′ homology arm with at least 90% sequence identity to the nucleotide sequence of SEQ ID NO: 127. In some embodiments, the 5′ homology arm comprises at least 95% sequence identity to the nucleotide sequence of SEQ ID NO: 119, and the 3′ homology arm comprises at least 95% to the nucleotide sequence of SEQ ID NO: 127. In some embodiments, the 5′ homology arm comprises the nucleotide sequence of SEQ ID NO: 119, and the 3′ homology arm comprises the nucleotide sequence of SEQ ID NO: 127.

In some embodiments, a nucleic acid for insertion into the TRAC locus comprises a 5′ homology arm with at least 90% sequence identity to the nucleotide sequence of SEQ ID NO: 130, and a 3′ homology arm with at least 90% sequence identity to the nucleotide sequence of SEQ ID NO: 138. In some embodiments, the 5′ homology arm comprises at least 95% sequence identity to the nucleotide sequence of SEQ ID NO: 130, and the 3′ homology arm comprises at least 95% to the nucleotide sequence of SEQ ID NO: 138. In some embodiments, the 5′ homology arm comprises the nucleotide sequence of SEQ ID NO: 130, and the 3′ homology arm comprises the nucleotide sequence of SEQ ID NO: 138.

In some embodiments, a nucleic acid for insertion into the FOXP3 locus comprises a 5′ homology arm with at least 90% sequence identity to the nucleotide sequence of SEQ ID NO: 141, and a 3′ homology arm with at least 90% sequence identity to the nucleotide sequence of SEQ ID NO: 149. In some embodiments, the 5′ homology arm comprises at least 95% sequence identity to the nucleotide sequence of SEQ ID NO: 141, and the 3′ homology arm comprises at least 95% to the nucleotide sequence of SEQ ID NO: 149. In some embodiments, the 5′ homology arm comprises the nucleotide sequence of SEQ ID NO: 141, and the 3′ homology arm comprises the nucleotide sequence of SEQ ID NO: 149.

In some embodiments, a nucleic acid for insertion into the FOXP3 locus comprises a 5′ homology arm with at least 90% sequence identity to the nucleotide sequence of SEQ ID NO: 152, and a 3′ homology arm with at least 90% sequence identity to the nucleotide sequence of SEQ ID NO: 160. In some embodiments, the 5′ homology arm comprises at least 95% sequence identity to the nucleotide sequence of SEQ ID NO: 152, and the 3′ homology arm comprises at least 95% to the nucleotide sequence of SEQ ID NO: 160. In some embodiments, the 5′ homology arm comprises the nucleotide sequence of SEQ ID NO: 152, and the 3′ homology arm comprises the nucleotide sequence of SEQ ID NO: 160.

In some embodiments, a nucleic acid for insertion into the FOXP3 locus comprises a 5′ homology arm with at least 90% sequence identity to the nucleotide sequence of SEQ ID NO: 163, and a 3′ homology arm with at least 90% sequence identity to the nucleotide sequence of SEQ ID NO: 171. In some embodiments, the 5′ homology arm comprises at least 95% sequence identity to the nucleotide sequence of SEQ ID NO: 163, and the 3′ homology arm comprises at least 95% to the nucleotide sequence of SEQ ID NO: 171. In some embodiments, the 5′ homology arm comprises the nucleotide sequence of SEQ ID NO: 163, and the 3′ homology arm comprises the nucleotide sequence of SEQ ID NO: 171.

In some embodiments, a nucleic acid for insertion into the FOXP3 locus comprises a 5′ homology arm with at least 90% sequence identity to the nucleotide sequence of SEQ ID NO: 174, and a 3′ homology arm with at least 90% sequence identity to the nucleotide sequence of SEQ ID NO: 183. In some embodiments, the 5′ homology arm comprises at least 95% sequence identity to the nucleotide sequence of SEQ ID NO: 174, and the 3′ homology arm comprises at least 95% to the nucleotide sequence of SEQ ID NO: 183. In some embodiments, the 5′ homology arm comprises the nucleotide sequence of SEQ ID NO: 174, and the 3′ homology arm comprises the nucleotide sequence of SEQ ID NO: 183.

In some embodiments, a nucleic acid for insertion into the FOXP3 locus comprises a 5′ homology arm with at least 90% sequence identity to the nucleotide sequence of SEQ ID NO: 186, and a 3′ homology arm with at least 90% sequence identity to the nucleotide sequence of SEQ ID NO: 194. In some embodiments, the 5′ homology arm comprises at least 95% sequence identity to the nucleotide sequence of SEQ ID NO: 186, and the 3′ homology arm comprises at least 95% to the nucleotide sequence of SEQ ID NO: 194. In some embodiments, the 5′ homology arm comprises the nucleotide sequence of SEQ ID NO: 186, and the 3′ homology arm comprises the nucleotide sequence of SEQ ID NO: 194.

In some embodiments, a nucleic acid for insertion into the FOXP3 locus comprises a 5′ homology arm with at least 90% sequence identity to the nucleotide sequence of SEQ ID NO: 197, and a 3′ homology arm with at least 90% sequence identity to the nucleotide sequence of SEQ ID NO: 205. In some embodiments, the 5′ homology arm comprises at least 95% sequence identity to the nucleotide sequence of SEQ ID NO: 197, and the 3′ homology arm comprises at least 95% to the nucleotide sequence of SEQ ID NO: 205. In some embodiments, the 5′ homology arm comprises the nucleotide sequence of SEQ ID NO: 197, and the 3′ homology arm comprises the nucleotide sequence of SEQ ID NO: 205.

In some embodiments, a nucleic acid for insertion into the FOXP3 locus comprises a 5′ homology arm with at least 90% sequence identity to the nucleotide sequence of SEQ ID NO: 208, and a 3′ homology arm with at least 90% sequence identity to the nucleotide sequence of SEQ ID NO: 217. In some embodiments, the 5′ homology arm comprises at least 95% sequence identity to the nucleotide sequence of SEQ ID NO: 208, and the 3′ homology arm comprises at least 95% to the nucleotide sequence of SEQ ID NO: 217. In some embodiments, the 5′ homology arm comprises the nucleotide sequence of SEQ ID NO: 208, and the 3′ homology arm comprises the nucleotide sequence of SEQ ID NO: 217.

### Genetically Modified Cells

Some aspects of the disclosure relate to genetically modified cells comprising two introduced nucleic acids in separate loci in the cell genome, one inserted into the TRAC locus, and another inserted into the FOXP3 locus, such that the cell is a dual-edited cell (i.e., having inserted nucleic acids at two distinct loci). The precise location of insertion will vary depending on the homology arms present on the nucleic acid targeting the locus. The first nucleic acid, targeting the TRAC locus, comprises 5′ and 3′ homology arms to direct insertion of the nucleic acid into the TRAC locus (e.g., by homology-directed repair (HDR) following cleavage of a DNA sequence in the TRAC locus by a nuclease). The second nucleic acid, targeting the FOXP3 locus, comprises 5′ and 3′ homology arms to direct insertion of the nucleic acid into the FOXP3 locus (e.g., by HDR following cleavage of a DNA sequence in the FOXP3 locus by a nuclease). Insertion of both nucleic acids into separate loci of the cell results in a dual-edited cell (i.e., a cell having inserted nucleic acids at two distinct loci).

In embodiments of the cells described herein, the modified TRAC locus comprises an inserted promoter that is operably linked to: (i) a nucleotide sequence encoding a first chemically induced signaling complex (CISC) component comprising: (a) an extracellular binding domain comprising or derived from an FK506-binding protein 12 (FKBP), (b) a transmembrane domain comprising or derived from an IL-2Rγ transmembrane domain, and (c) an intracellular signaling domain comprising or derived from an IL-2Rγ cytoplasmic domain; (ii) a nucleotide sequence encoding a full-length TCRβ chain; and (iii) a nucleotide sequence encoding at least a portion of a heterologous TCRα chain. The nucleotide sequence encoding the heterologous TCRα chain is inserted in-frame with an endogenous sequence encoding an endogenous TCRα portion (e.g. a TCRα constant domain), such that translation of the expressed mRNA produces a TCRα chain that associates with the heterologous TCRβ chain to form a TCR. Because the antigen-binding regions of the TCRα chain are encoded by the inserted nucleic acid, the specificity of the TCR is governed by the inserted nucleic acid. In cells described herein, the TCR encoded by the inserted nucleic acid binds to a T1D-associated antigen. Thus, in the modified TRAC locus, the inserted promoter initiates transcription of the operably linked sequences, such that the FKBP-IL2Rγ CISC component, and a T1D-associated antigen-specific TCR formed by the heterologous TCRβ chain and TCRα chain comprising the heterologous portion encoded by the inserted nucleic acid, are expressed from the modified TRAC locus.

In embodiments of the cells described herein, the modified FOXP3 locus comprises an inserted promoter that is operably linked to: (i) a nucleotide sequence encoding a first chemically induced signaling complex (CISC) component comprising: (a) an extracellular binding domain comprising or derived from an FKBP-rapamycin-binding (FRB) domain of mTOR, (b) a transmembrane domain comprising or derived from an IL-2Rβ transmembrane domain, and (c) an intracellular signaling domain comprising or derived from an IL-2Rβ cytoplasmic domain; (ii) a nucleotide sequence encoding a cytosolic FRB domain that lacks a transmembrane domain; and (iii) a nucleotide sequence encoding FoxP3. The promoter is inserted into the FOXP3 locus downstream from the Treg-specific demethylated region in the FOXP3 locus (e.g., homology to a sequence within or up to 2,000 nucleotides upstream from exon 2, the first coding exon of the FOXP3 gene). Insertion of the promoter downstream from the TSDR, which destabilizes FOXP3 expression in inflammatory conditions, allows the inserted promoter to initiate transcription of FoxP3-encoding mRNA independently of the endogenous FOXP3 promoter, which is upstream from the TSDR. Thus, in the modified FOXP3 locus the inserted promoter initiates transcription of the operably linked sequences, such that the FRB-Il2Rβ CISC component, cytosolic FRB component, and FoxP3 are expressed from the FOXP3 locus.

Having modified TRAC and FOXP3 loci as described in the preceding paragraphs, the dual-edited cell stably expresses: (i) first and second CISC components that form a heterodimer in the presence of rapamycin, resulting in IL-2R signal transduction via dimerization of the cytoplasmic IL-2Rβ and IL-2Rγ domains; (ii) a cytosolic FRB domain that binds intracellular rapamycin, preventing its interaction with mTOR; (iii) FoxP3, providing for a stable Treg phenotype; and (iv) a TCR specific to a T1D-associated antigen. Thus, the cells described herein are stable Treg cells with T1D-associated antigen specificity, which can be induced to proliferate using rapamycin. Moreover, separation of the nucleotide sequences encoding first and second CISC components into distinct loci allows rapamycin to induce proliferation selectively in cells expressing both CISC components (and thus expressing the T1D antigen-specific TCR and FoxP3 due to modification of both loci). Thus, dual-edited cells may readily be selected and proliferated in vitro to produce a population of stable Treg cells having T1D-associated antigen specificity for treating T1D. Additionally, engraftment and proliferation of such stable Treg cells may be supported in vivo by administering rapamycin to a subject.

### Promoters

Nucleic acids inserted into genomes of genetically modified cells described herein each comprise a promoter operably linked to one or more nucleotide sequences inserted into the FOXP3 or TRAC locus. A promoter is “operably linked” to a sequence if it is capable of initiating transcription of the operably linked sequence (e.g., by recruitment of RNA polymerase). The inserted promoters of the modified TRAC and FOXP3 loci may be any promoter known in the art. In some embodiments, the inserted heterologous promoter is active, promoting transcription of RNA, even under pro-inflammatory conditions. In some embodiments, the promoter is a constitutive promoter. Constitutive promoters may be strong promoters, which promote transcription at a higher rate than an endogenous promoter, or weak promoters, which promote transcription at a lower rate than a strong or endogenous promoter. In some embodiments, the constitutive promoter is a strong promoter. In some embodiments, the heterologous promoter is an inducible promoter. Inducible promoters promote transcription of an operably linked sequence in response to the presence of an activating signal, or the absence of a repressor signal. In some embodiments, the inducible promoter is inducible by a drug or steroid.

In some embodiments, the promoters inserted into the TRAC locus and FOXP3 locus the cell are different promoters. In other embodiments, the TRAC and FOXP3 loci both comprise the same promoter. In some embodiments, the TRAC and FOXP3 loci both comprise an MND promoter. In embodiments where the TRAC and FOXP3 loci both comprise the same promoter, the promoter sequences may be identical between both TRAC and FOXP3 loci. Alternatively, the promoter sequence of the modified TRAC locus may comprise one or more mutations (e.g., insertions, deletions, substitutions) relative to the promoter sequence of the FOXP3 locus. In some embodiments, the MND promoter of the TRAC and/or FOXP3 locus comprises at least 90% sequence identity to the nucleotide sequence of SEQ ID NO: 220. In some embodiments, the MND promoter of the TRAC and/or FOXP3 nucleotide comprises at least 95% sequence identity to the nucleotide sequence of SEQ ID NO: 220. In some embodiments, each of the TRAC and FOXP3 loci comprises an MND promoter having the nucleotide sequence of SEQ ID NO: 220.

In some embodiments, a STOP codon is present upstream or within the first five nucleotides of the promoter inserted into the TRAC locus. In some embodiments, a STOP codon is present upstream or within the first five nucleotides of the promoter inserted into the FOXP3 locus. The presence of a STOP codon upstream from, within, or overlapping with the first five nucleotides of the promoter is expected to terminate translation of mRNAs that may be transcribed from an endogenous promoter upstream in the modified TRAC or FOXP3 locus, thereby inhibiting expression of inserted coding sequences (e.g., encoding CISC components, heterologous TCRβ or TCRα chains, or FoxP3) under control of the endogenous promoter. In some embodiments, the STOP codon is in-frame with one or more upstream START codons, such that mRNA produced following transcription from the endogenous upstream promoter is not translated past the STOP codon.

### Chemically Induced Signaling Complex (CISC)

Embodiments of the genetically modified cells described herein comprise a genome in which each of the TRAC and FOXP3 loci comprises a nucleotide sequence encoding a chemically induced signaling complex (CISC) component, each CISC component comprising an extracellular domain that binds rapamycin, a transmembrane domain, and an intracellular domain comprising or derived from an interleukin-2 receptor (IL-2R) cytoplasmic domain. In some embodiments, the TRAC locus encodes a first CISC component comprising (i) an extracellular binding domain comprising an FK506-binding protein 12 (FKBP) domain, (ii) a transmembrane domain comprising or derived from an IL-2Rγ transmembrane domain, and (iii) an intracellular domain comprising or derived from an IL-2Rγ cytoplasmic domain; and the FOXP3 locus encodes a first CISC component comprising (i) an extracellular binding domain comprising an FKBP-rapamycin-binding domain, (ii) a transmembrane domain comprising or derived from an IL-2Rβ transmembrane domain, and (iii) an intracellular domain comprising or derived from an IL-2Rβ cytoplasmic domain. A domain of a CISC component (e.g., transmembrane domain of the first CISC component) is “derived from” a given domain of an IL-2R polypeptide (e.g., IL-2Rγ) if it comprises at least 90% sequence identity to a wild-type (naturally occurring) amino acid sequence of the domain (e.g., a naturally occurring IL-2Rγ transmembrane domain).

Expression of CISC components in a cell allows selective induction of IL-2 signal transduction in a cell by manipulation of the presence and/or concentration of the rapamycin. Such controllable induction of signaling allows, for example, selective expansion of cells expressing both CISC components, where the IL-2 signal transduction event results in proliferation of the cell. In some embodiments, where two loci are modified, each containing an inserted nucleotide encoding a different CISC component, such selective expansion allows for selection of cells that contain both modified loci, as contacting a cell comprising only one CISC component with rapamycin would not induce dimerization with the absent second CISC component, and thus not lead to IL-2 signal transduction.

Non-limiting examples of intracellular signaling domains include IL-2Rβ and IL-2Rγ cytoplasmic domains and functional derivatives thereof. In some embodiments, an intracellular signaling domain of the first CISC component comprises an IL-2Rγ domain or a functional derivative thereof, and an intracellular signaling domain of a second CISC component comprises an IL-2Rβ cytoplasmic domain or a functional derivative thereof. In some embodiments, dimerization of the first and second CISC components induces phosphorylation of JAK1, JAK3, and/or STAT5 in the cell. In some embodiments, dimerization of the first and second CISC components induces proliferation of the cell.

Non-limiting examples of transmembrane domains include IL-2Rβ and IL-2Rγ transmembrane domains and functional derivatives thereof. In some embodiments, the transmembrane domain of a CISC component is derived from the same protein as the intracellular signaling domain of the CISC component (e.g., a CISC component comprising an IL-2Rβ intracellular domain comprises an IL-2Rβ transmembrane domain). In some embodiments, one CISC component comprises an IL-2Rβ transmembrane domain, and the other CISC component comprises an IL-2Rγ transmembrane domain.

Non-limiting examples of extracellular binding domains capable of binding to rapamycin include an FK506-binding protein (FKBP) domain and an FKBP-rapamycin-binding (FRB) domain. FKBP and FRB domains are capable of binding to rapamycin, such as those described below, to form a heterodimer. In some embodiments, an extracellular binding domain of one CISC component comprises an FKBP domain, and an extracellular binding domain of the other CISC component comprises an FRB domain. In some embodiments, the CISC components form a heterodimer in the presence of rapamycin. In some embodiments, the FRB domain comprises a threonine at a position corresponding to amino acid 2098 of wild-type mTOR having the amino acid sequence of SEQ ID NO: 236. Mutation of this amino acid increases the affinity of mTOR for compounds having related structures to rapamycin, but decreases the affinity of mTOR for rapamycin itself. Thus, inclusion of a threonine at this position maintains the ability of mTOR to bind to rapamycin. The amino acid of a CISC component or FRB domain that “corresponds to” amino acid 2098 of wild-type mTOR may be determined by aligning a candidate sequence of a CISC component or FRB domain to SEQ ID NO: 236 (e.g., by BLAST or another alignment algorithm known in the art), with the amino acid aligned to amino acid 2098 of SEQ ID NO: 236 being the amino acid that “corresponds to” amino acid 2098 of SEQ ID NO: 236.

Each of the extracellular binding domains, transmembrane domains, and intracellular signaling domains of the CISC components described herein may be connected to another domain of the same CISC component by a linker. Linkers are known in the art. In some embodiments, the linker comprises 1, 2, 3, 4, 5, 6, 7, 8, 9 or 10 amino acids, such as glycines, or a number of amino acids, such as glycine, within a range defined by any two of the aforementioned numbers. In some embodiments, the glycine spacer comprises at least 3 glycines. In some embodiments, the glycine spacer comprises a sequence set forth as GSG, GGGS (SEQ ID NO: 229), GGGSGGG (SEQ ID NO: 230) or GGG. In some embodiments, the glycine spacer comprises the amino acid sequence GSG.

An extracellular binding domain may be connected to a transmembrane domain by a hinge domain. A hinge refers to a domain that links the extracellular binding domain to the transmembrane domain, and may confer flexibility to the extracellular binding domain. In some embodiments, the hinge domain positions the extracellular binding domain close to the plasma membrane to minimize the potential for recognition by antibodies or binding fragments thereof. In some embodiments, the extracellular binding domain is located N-terminal to the hinge domain. In some embodiments, the hinge domain may be natural or synthetic.

In some embodiments, the first and second CISC components form a heterodimer in the presence of rapamycin. In some embodiments, the first and second CISC components form a heterodimer in the presence of a compound that produced in vivo by metabolism of a rapalog. In some embodiments, the compound produced by in vivo metabolism of the rapalog is rapamycin. Non-limiting examples of rapalogs include everolimus, CCI-779, C20-methallylrapamycin, C16-(S)-3-methylindolerapamycin, C16-iRap, C16-(S)-7-methylindolerapamycin, AP21967, C16-(S)Butylsulfonamidorapamycin, AP23050, sodium mycophenolic acid, benidipine hydrochloride, AP1903, and AP23573, and metabolites or derivatives thereof.

In some embodiments, the FOXP3 locus further comprises a nucleotide sequence encoding a third CISC component that binds to rapamycin. Such CISC components are useful, for example, for binding to intracellular rapamycin, thereby preventing the bound rapamycin from interacting with other intracellular molecules or structures (e.g., preventing rapamycin from interacting with mTOR). In some embodiments, the third CISC component is a soluble protein that does not comprise a transmembrane domain. In some embodiments, the third CISC component comprises an intracellular FRB domain. In some embodiments, a third CISC component is a soluble protein comprising an FRB domain and lacking a transmembrane domain.

In some embodiments, a CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 66 or 71. In some embodiments, one or more CISC components further comprise a signal peptide. The signal peptide may be any signal peptide known in the art that directs the translated CISC component to the cell membrane. In some embodiments, each of the first and second CISC components comprises an LCN2 signal peptide. In some embodiments, each of the first and second CISC components comprises a signal peptide comprising the amino acid sequence of SEQ ID NO: 73

In some embodiments, one CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 66, and the other CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 71. In some embodiments, each CISC component further comprises a signal peptide, which may have the same or different amino acid sequences. The signal peptides may be any signal peptide known in the art that directs the translated CISC component to the cell membrane.

In some embodiments, a third CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 72. In some embodiments, a third CISC component consists of an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 72. In some embodiments, the third CISC component comprises the amino acid sequence of SEQ ID NO: 72. In some embodiments, the third CISC component consists of the amino acid sequence of SEQ ID NO: 72. In some embodiments, the third CISC component does not comprise a signal peptide. In some embodiments, the third CISC component does not comprise a transmembrane domain.

### T Cell Receptors (TCRs)

In some embodiments of the cells described herein, the TRAC locus is edited by insertion of a nucleic acid comprising a promoter operably linked to a nucleotide sequence encoding a full-length TCRβ protein, and to a nucleotide sequence encoding at least a portion of a TCRα protein, such as TCRα variable and TCRα joining (TRAJ) regions that form the portion of a TCRα protein responsible for antigen-specificity. In some embodiments the inserted nucleotide sequence encoding the TCRα variable and joining regions is in-frame with the endogenous nucleotide sequence encoding a portion of the TCRα constant domain, such that the inserted heterologous promoter initiates transcription of a sequence encoding a heterologous TCRβ protein and a sequence encoding a TCRα protein comprising heterologous TRAV/TRAJ amino acid sequences and an endogenous TCRα constant domain. This embodiment utilizes the endogenous 3′ regulatory region from the endogenous TRAC locus.

Genetically modified cells described herein express a T cell receptor specific to a type 1 diabetes (T1D)-associated antigen. T cell receptors for expression by genetically modified cells are described herein under the heading “Methods for producing genetically modified cells” and subheading “T cell receptors (TCRs).” In certain embodiments, a sequence in the cell genome encoding a TCR is codon-optimized to enhance expression in a particular host cell, such as, for example, a cell of the immune system, a hematopoietic stem cell, a T cell, a primary T cell, a T cell line, a NK cell, or a natural killer T cell. See, e.g., Scholten et al., C/in Immunol. 2006. 119:135.

In some embodiments, a modified TRAC locus of a genetically modified cell described herein encodes a TCRβ chain and at least a portion of a TCRα chain that, expressed in combination, form a T1D2 TCR that binds to a peptide of IGRP(305-234). In other embodiments, a TCRβ chain and full-length TCRα chain, a portion of which is encoded by a modified TRAC locus described herein, form a T1D4 TCR that binds a peptide of IGRP(241-260). In other embodiments, a TCRβ chain and full-length TCRα chain, a portion of which is encoded by an inserted nucleotide sequence described herein, form a T1D5-1 TCR that binds a peptide of IGRP(305-324). In some embodiments, the peptide of IGRP(305-324) is recognized when bound to HLA-DRB1*0401. In some embodiments, the peptide of IGRP(241-260) is recognized when bound to HLA-DRB1*0401.

In some embodiments, a TCR formed by a TCRβ chain and (at least a portion of) the TCRα chain encoded by a modified TRAC locus of a genetically modified cell described herein comprises a TCRα variable (Vα) domain having three complementarity determining regions (CDRs) of αCDR1, αCDR2, and αCDR3; and a TCRβ variable (Vβ) domain having three CDRs of βCDR1, βCDR2, and βCDR3. Representative amino acids of CDRs of TCRs described herein are shown in Table 1, and nucleotide sequences encoding the same are shown in Table 2. In some embodiments: (i) αCDR1 comprises SEQ ID NO: 1, (ii) αCDR2 comprises SEQ ID NO: 2, (iii) αCDR3 comprises SEQ ID NO: 3, (iv) βCDR1 comprises SEQ ID NO: 4, (v) βCDR2 comprises SEQ ID NO: 5, and (vi) βCDR3 comprises SEQ ID NO: 6. In some embodiments: (i) αCDR1 comprises SEQ ID NO: 11, (ii) αCDR2 comprises SEQ ID NO: 12, (iii) αCDR3 comprises SEQ ID NO: 13, (iv) βCDR1 comprises SEQ ID NO: 14, (v) βCDR2 comprises SEQ ID NO: 15, and (vi) βCDR3 comprises SEQ ID NO: 16. In some embodiments: (i) αCDR1 comprises SEQ ID NO: 21, (ii) αCDR2 comprises SEQ ID NO: 22, (iii) αCDR3 comprises SEQ ID NO: 23, (iv) βCDR1 comprises SEQ ID NO: 24, (v) βCDR2 comprises SEQ ID NO: 25, and (vi) βCDR3 comprises SEQ ID NO: 26. In other embodiments, each of the set of αCDR1, αCDR2, αCDR3, βCDR1, βCDR2, and βCDR3 may have an amino acid sequence having at least 80%, at least 85%, at least 90%, at least 91%, at least 92%, at least 93%, at least 94%, at least 95%, at least 96%, at least 97%, at least 98%, or at least 99% sequence identity to the respective amino acid sequences in any of the aforementioned combinations of amino acid sequences.

In some embodiments, Vα comprises SEQ ID NO: 7 and Vβ comprises SEQ ID NO: 8. In some embodiments, Vα comprises SEQ ID NO: 17 and Vβ comprises SEQ ID NO: 18. In some embodiments, Vα comprises SEQ ID NO: 27 and Vβ comprises SEQ ID NO: 28. In other embodiments, each of the pair of Vα and Vβ may have an amino acid sequence having at least 80%, at least 85%, at least 90%, at least 91%, at least 92%, at least 93%, at least 94%, at least 95%, at least 96%, at least 97%, at least 98%, or at least 99% sequence identity to the respective amino acid sequence any of the aforementioned combinations of amino acid sequences.

In some embodiments, the TCRα chain comprises SEQ ID NO: 9 and the TCRβ chain comprises SEQ ID NO: 10. In some embodiments, the TCRα chain comprises SEQ ID NO: 19 and the TCRβ chain comprises SEQ ID NO: 20. In some embodiments, the TCRα chain comprises SEQ ID NO: 29 and the TCRβ chain comprises SEQ ID NO: 30. In other embodiments, each of the pair of TCRα and TCRβ chains may have an amino acid sequence having at least 80%, at least 85%, at least 90%, at least 91%, at least 92%, at least 93%, at least 94%, at least 95%, at least 96%, at least 97%, at least 98%, or at least 99% sequence identity to the respective amino acid sequence of any of the aforementioned combinations of amino acid sequences.

### FOXP3 Locus Modification

In some embodiments of the genetically modified cells described herein, the FOXP3 locus comprises an inserted promoter operably linked to a nucleotide sequence encoding at least a portion of the endogenous FoxP3 protein. The inserted promoter is introduced into the genome downstream from the Treg-specific demethylated region (TSDR) of the FOXP3 locus. In unmodified cells, the TSDR epigenetically regulates expression of FoxP3, inhibiting FoxP3 production in cells exposed to inflammatory conditions, which may result in loss of FoxP3 expression and conversion of unmodified Treg cells to a T effector (Teff) phenotype. Insertion of a promoter downstream from the TSDR bypasses TSDR-mediated regulation of FOXP3 expression, thereby providing stable production of FoxP3 even in inflammatory conditions.

The heterologous promoter may be inserted at any position downstream from the endogenous promoter (e.g., downstream from the TSDR) and upstream from or within the first coding exon of the FOXP3 coding sequence. This first coding exon is known in the art as exon 2, as it is the second exon present in pre-mRNA transcribed from the endogenous FOXP3 promoter, and the first coding exon because it is this exon, not exon 1 (the first exon of FOXP3-encoding pre-mRNA) that contains the start codon that initiates translation of wild-type FoxP3. In some embodiments, the heterologous promoter is inserted 1-10,000, 10-1,000, 10-100, 10-5,000, 20-4,000, 30-3,000, 40-2,000, 50-1,000, 60-750, 70-500, 80-400, 90-300, 100-200, 1-1,000, 1,000-2,000, 2,000-3,000, 3,000-4,000, 4,000-5,000, 5,000-6,000, 6,000-7,000, 7,000-8,000, 8,000-9,000, or 9,000-10,000 nucleotides downstream from the TSDR of FOXP3. In some embodiments, the heterologous promoter is inserted 1-10,000, 10-1,000, 10-100, 10-5,000, 20-4,000, 30-3,000, 40-2,000, 50-1,000, 60-750, 70-500, 80-400, 90-300, 100-200, 1-1,000, 1,000-2,000, 2,000-3,000, 3,000-4,000, 4,000-5,000, 5,000-6,000, 6,000-7,000, 7,000-8,000, 8,000-9,000, or 9,000-10,000 nucleotides upstream from the first coding exon of the FOXP3 coding sequence. In some embodiments, the heterologous promoter is inserted into the first coding exon, such that a synthetic first coding exon is created, where the synthetic first coding exon differs from the endogenous first coding exon but still comprises a start codon that is in-frame with the FOXP3 coding sequence of downstream FOXP3 exons.

### 2A Motifs and Linkers

Some embodiments of modified TRAC and/or modified FOXP3 loci of genetically modified cells described herein encoding multiple polypeptides or portions thereof may contain intervening nucleotide sequences encoding a 2A motifs. 2A motifs are known in the art, and are useful for promoting production of multiple polypeptides from translation of a single nucleotide sequence. See, e.g., Kim et al., PLoS ONE. 2011. 6:e18556. In some embodiments, the 2A motif is translated, and self-cleavage of the polypeptide occurs following translation, resulting in release of separate polypeptides. In other embodiments, the nucleotide sequence encoding the 2A motif causes the ribosome to progress along an mRNA without incorporating an encoded amino acid of the 2A motif, resulting in release of the first polypeptide (e.g., first FKBP-IL2Rγ CISC component), and allowing translation initiation of a second polypeptide (e.g., TCRβ chain).

In some embodiments, nucleotide sequences encoding a 2A motif are present in-frame with and between each pair of nucleotide sequences encoding (i) the first (FKBP-IL2Rγ) CISC component; (ii) the TCRβ chain; and (iii) the TCRα chain or portion thereof. Thus, the heterologous promoter (e.g., MND promoter) initiates transcription of a single mRNA encoding each of the CISC component, TCRβ chain, and TCRα chain, with intervening 2A motifs allowing production of each as a separate polypeptide. In some embodiments, a nucleotide sequence encoding a 2A motif is in-frame with and between each pair of nucleotide sequences encoding (i) the second (FKBP-IL2Rγ) CISC component; (ii) the cytosolic FRB domain; and (iii) FoxP3. Thus, the heterologous promoter (e.g., MND promoter) initiates transcription of a single mRNA encoding each of the CISC component, cytosolic FRB domain, and FoxP3, with intervening 2A motifs allowing production of each as a separate polypeptide.

The 2A motifs encoded by nucleotide sequences between each pair of sequences encoding two polypeptides (e.g., sequences encoding an FKBP-IL2Rγ CISC component and TCRβ chain; TCRβ chain and portion of α chain) may be any 2A motif known in the art. In some embodiments, the encoded 2A motifs between each pair of nucleotide sequences encoding distinct polypeptides may be independently selected from the group consisting of F2A, P2A, T2A, E2A. In some embodiments, a first encoded 2A motif and second encoded 2A motif in a modified TRAC and/or FOXP3 locus are different 2A motifs. Use of different 2A motifs in the same modified TRAC and/or FOXP3 locus reduces the probability of internal recombination, which may result in the nucleotide sequence between the recombined 2A motifs being excised from the chromosome. In some embodiments, a nucleotide sequence encoding a first 2A motif has no more than 90% sequence identity to a nucleotide sequence encoding a second 2A motif on the same modified TRAC and/or FOXP3 locus. In some embodiments, a nucleotide sequence encoding a first 2A motif has no more than 80% sequence identity to a nucleotide sequence encoding a second 2A motif on the same modified TRAC and/or FOXP3 locus. In some embodiments, a nucleotide sequence encoding a first 2A motif has no more than 70% sequence identity to a nucleotide sequence encoding a second 2A motif on the same modified TRAC and/or FOXP3 locus. In some embodiments, a nucleotide sequence encoding a first 2A motif has no more than 60% sequence identity to a nucleotide sequence encoding a second 2A motif on the same modified TRAC and/or FOXP3 locus. In some embodiments, a nucleotide sequence encoding a first 2A motif has no more than 50% sequence identity to a nucleotide sequence encoding a second 2A motif on the same modified TRAC and/or FOXP3 locus. In some embodiments, a first 2A motif is a T2A motif, and the second motif is a P2A motif.

In other embodiments, the first and second 2A motifs encoded by nucleotide sequences on the modified TRAC and/or FOXP3 locus are the same 2A motif. In some embodiments, a modified TRAC and/or FOXP3 locus comprises a nucleotide sequence encoding a first P2A motif, and a second nucleotide sequence encoding a second P2A motif, with the nucleotide sequence encoding the first P2A motif comprising at least 80% sequence identity to the nucleotide sequence encoding the second P2A motif. In some embodiments, the first and second nucleotide sequences encoding the first and second P2A motifs comprise the same nucleotide sequences.

In some embodiments, the modified TRAC locus comprises: (i) a sequence encoding a T2A motif between the sequence encoding the first CISC component and the sequence encoding the TCRβ chain; and (ii) a sequence encoding a P2A motif between the sequence encoding the TCRβ chain and heterologous TCRα chain portion.

In some embodiments, the modified FOXP3 locus comprises: (i) a sequence encoding a P2A motif between the sequence encoding the second CISC component and the sequence encoding the cytosolic FRB domain; and (ii) a second sequence encoding a second P2A motif between the sequence encoding the cytosolic FRB domain and the sequence encoding FoxP3.

In some embodiments, a polypeptide (e.g., CISC components and/or TCRβ chains) encoded by a nucleotide sequence inserted into the modified TRAC or FOXP3 locus comprises a C-terminal linker. Incorporation of such a linker may, for example, improve efficiency of cleavage in 2A motifs and/or prevent cleavage of a 2A motif from excising amino acids of the encoded CISC component or TCRβ chain. In some embodiments, the encoded first CISC component comprises a C-terminal linker. In some embodiments, the encoded second CISC component comprises a C-terminal linker. In some embodiments, the encoded cytosolic FRB domain component comprises a C-terminal linker. In some embodiments, the encoded TCRβ chain comprises a C-terminal linker.

Linkers at the C-terminus of encoded polypeptides may be any linker known in the art. In some embodiments, the linker comprises 1, 2, 3, 4, 5, 6, 7, 8, 9 or 10 amino acids, such as glycines, or a number of amino acids, such as glycine, within a range defined by any two of the aforementioned numbers. In some embodiments, the linker comprises at least 3 glycines. In some embodiments, the linker comprises a sequence set forth as GSG, GGGS (SEQ ID NO: 229), GGGSGGG (SEQ ID NO: 230) or GGG. In some embodiments, the linker comprises the amino acid sequence GSG. In some embodiments, each of the first CISC component, second CISC component, cytosolic FRB domain, and TCRβ chain comprises a C-terminal linker having the amino acid sequence GSG.

In some embodiments, a modified TRAC locus comprises a nucleotide sequence having at least 90% sequence identity to any one of SEQ ID NOs: 94, 106, 117, 128, and 139. In some embodiments, the nucleotide sequence comprises at least 95% sequence identity to any one of SEQ ID NOS: 94, 106, 117, 128, and 139. In some embodiments, the nucleotide sequence comprises any one of SEQ ID NOS: 94, 106, 117, 128, and 139. In some embodiments, a modified TRAC locus comprises the nucleotide sequence of SEQ ID NO: 94. In some embodiments, a modified TRAC locus comprises the nucleotide sequence of SEQ ID NO: 106. In some embodiments, a modified TRAC locus comprises the nucleotide sequence of SEQ ID NO: 117. In some embodiments, a modified TRAC locus comprises the nucleotide sequence of SEQ ID NO: 128. In some embodiments, a modified TRAC locus comprises the nucleotide sequence of SEQ ID NO: 139.

In some embodiments, a modified FOXP3 locus comprises a nucleotide sequence having at least 90% sequence identity to any one of SEQ ID NOs: 150, 161, 172, 184, 195, 206, and 218. In some embodiments, the nucleotide sequence comprises at least 95% sequence identity to any one of SEQ ID NOS: 150, 161, 172, 184, 195, 206, and 218. In some embodiments, the nucleotide sequence comprises any one of SEQ ID NOS: 150, 161, 172, 184, 195, 206, and 218. In some embodiments, the modified FOXP3 locus comprises the nucleotide sequence of SEQ ID NO: 150. In some embodiments, the modified FOXP3 locus comprises the nucleotide sequence of SEQ ID NO: 161. In some embodiments, the modified FOXP3 locus comprises the nucleotide sequence of SEQ ID NO: 172. In some embodiments, the modified FOXP3 locus comprises the nucleotide sequence of SEQ ID NO: 184. In some embodiments, the modified FOXP3 locus comprises the nucleotide sequence of SEQ ID NO: 195. In some embodiments, the modified FOXP3 locus comprises the nucleotide sequence of SEQ ID NO: 206. In some embodiments, the modified FOXP3 locus comprises the nucleotide sequence of SEQ ID NO: 218.

### Systems for Producing Genetically Modified Cells

Some aspects of the disclosure relate to systems for producing a genetically modified cell, comprising two nucleic acids, one with homology to the TRAC locus, and another with homology to the FOXP3 locus of the cell, such that both loci may be edited by insertion of the nucleic acids into respective loci. The first nucleic acid, targeting the TRAC locus, comprises 5′ and 3′ homology arms to direct insertion of the nucleic acid into the TRAC locus (e.g., by homology-directed repair (HDR) following cleavage of a DNA sequence in the TRAC locus by a nuclease). The second nucleic acid, targeting the FOXP3 locus, comprises 5′ and 3′ homology arms to direct insertion of the nucleic acid into the FOXP3 locus (e.g., by HDR following cleavage of a DNA sequence in the FOXP3 locus by a nuclease). Insertion of both nucleic acids into separate loci of the cell results in a dual-edited cell (i.e., a cell having inserted nucleic acids at two distinct loci).

In embodiments of the systems described herein, the nucleic acid targeted for insertion into the TRAC locus comprises a promoter that is operably linked to: (i) a nucleotide sequence encoding a first chemically induced signaling complex (CISC) component comprising: (a) an extracellular binding domain comprising or derived from an FK506-binding protein 12 (FKBP), (b) a transmembrane domain comprising or derived from an IL-2Rγ transmembrane domain, and (c) an intracellular signaling domain comprising or derived from an IL-2Rγ cytoplasmic domain; (ii) a nucleotide sequence encoding a full-length TCRβ chain; and (iii) a nucleotide sequence encoding at least a portion of a TCRα chain. The nucleotide sequence encoding the heterologous TCRα is inserted in-frame with an endogenous sequence encoding an endogenous TCRα portion (e.g. a TCRα constant domain), such that translation of the expressed mRNA produces a TCRα chain that associates with the heterologous TCRβ chain to form a TCR. Because the antigen-binding regions of the TCRα chain are encoded by the inserted nucleic acid, the specificity of the TCR is governed by the inserted nucleic acid. In the systems described herein, the TCR encoded by the inserted nucleic acid binds to a T1D-associated antigen. Following insertion into the TRAC locus, the promoter initiates transcription (and thereby promotes expression) of the operably linked sequences, such that the FKBP-IL2Rγ CISC component, and a T1D-associated antigen-specific TCR formed by the heterologous TCRβ chain and TCRα chain comprising the heterologous portion encoded by the inserted nucleic acid, are expressed from the TRAC locus.

In embodiments of the systems described herein, the nucleic acid targeted for insertion into the FOXP3 locus comprises a promoter that is operably linked to: (i) a nucleotide sequence encoding a first chemically induced signaling complex (CISC) component comprising: (a) an extracellular binding domain comprising or derived from an FKBP-rapamycin-binding (FRB) domain of mTOR, (b) a transmembrane domain comprising or derived from an IL-2Rβ transmembrane domain, and (c) an intracellular signaling domain comprising or derived from an IL-2Rβ cytoplasmic domain; (ii) a nucleotide sequence encoding a cytosolic FRB domain that lacks a transmembrane domain; and (iii) a 3′ homology arm with homology to a sequence in the FOXP3 locus that is downstream from the Treg-specific demethylated region in the FOXP3 locus (e.g., homology to a sequence within or up to 2,000 nucleotides upstream from exon 2, the first coding exon of the FOXP3 gene). Insertion in this manner downstream from the TSDR, which destabilizes FOXP3 expression in inflammatory conditions, allows the inserted promoter to initiate transcription of FoxP3-encoding mRNA independently of the endogenous FOXP3 promoter, which is upstream from the TSDR. Following insertion into the FOXP3 locus, the promoter initiates transcription of the operably linked sequences, such that the FRB-Il2Rβ CISC component, cytosolic FRB component, and FoxP3 are expressed from the FOXP3 locus.

Following insertion of both nucleic acids into the TRAC and FOXP3 loci, respectively, the dual-edited cell stably expresses: (i) first and second CISC components that form a heterodimer in the presence of rapamycin, resulting in IL-2R signal transduction via dimerization of the cytoplasmic IL-2Rβ and IL-2Rγ domains; (ii) a cytosolic FRB domain that binds intracellular rapamycin, preventing its interaction with mTOR; (iii) FoxP3, providing for a stable Treg phenotype; and (iv) a TCR specific to a T1D-associated antigen. Thus, the systems described herein provide for stable Treg cells with T1D-associated antigen specificity, which can be induced to proliferate using rapamycin. Moreover, separation of the nucleotide sequences encoding first and second CISC components onto distinct nucleic acids allows rapamycin to induce proliferation selectively in cells expressing both CISC components (and thus expressing the T1D antigen-specific TCR and FoxP3 due to insertion of both nucleic acids). Thus, dual-edited cells may readily be selected and proliferated in vitro to produce a population of stable Treg cells having T1D-associated antigen specificity for treating T1D. Additionally, engraftment and proliferation of such stable Treg cells may be supported in vivo by administering rapamycin to a subject.

### Promoters

Nucleic acids for targeted insertion into cell genomes using systems described herein each comprise a promoter operably linked to one or more nucleotide sequences on the nucleic acid. A promoter is “operably linked” to a sequence if it is capable of initiating transcription of the operably linked sequence (e.g., by recruitment of RNA polymerase). The promoters of the first and second nucleic acids may be any promoter known in the art. In some embodiments, the heterologous promoter on the introduced nucleic acid is active, promoting transcription of RNA, even under pro-inflammatory conditions. In some embodiments, the promoter is a constitutive promoter. Constitutive promoters may be strong promoters, which promote transcription at a higher rate than an endogenous promoter, or weak promoters, which promote transcription at a lower rate than a strong or endogenous promoter. In some embodiments, the constitutive promoter is a strong promoter. In some embodiments, the heterologous promoter is an inducible promoter. Inducible promoters promote transcription of an operably linked sequence in response to the presence of an activating signal, or the absence of a repressor signal. In some embodiments, the inducible promoter is inducible by a drug or steroid.

In some embodiments, the promoters of the first and second nucleic acids for insertion into cell genomes are different promoters. In other embodiments, the first and second nucleic acid both comprise the same promoter. In some embodiments, the first and second nucleic acid both comprise an MND promoter. In embodiments where the first and second nucleic acid both comprise the same promoter, the promoter sequences may be identical between both nucleic acids. Alternatively, the promoter sequence of the first nucleic acid may comprise one or more mutations (e.g., insertions, deletions, substitutions) relative to the promoter sequence of the second nucleic acid. In some embodiments, the MND promoter of the first and/or second nucleic acid comprises at least 90% sequence identity to the nucleic acid sequence of SEQ ID NO: 220. In some embodiments, the MND promoter of the first and/or second nucleic acid comprises at least 95% sequence identity to the nucleic acid sequence of SEQ ID NO: 220. In some embodiments, each of the first and second nucleic acids comprises an MND promoter having the nucleic acid sequence of SEQ ID NO: 220.

In some embodiments, a STOP codon is present upstream or within the first five nucleotides of the promoter on the first nucleic acid for insertion into the TRAC locus. In some embodiments, a STOP codon is present upstream or within the first five nucleotides of the promoter on the second nucleic acid for insertion into the FOXP3 locus. The presence of a STOP codon upstream from, within, or overlapping with the first five nucleotides of the promoter is expected to terminate translation of mRNAs that may be transcribed from an endogenous promoter upstream in the modified TRAC or FOXP3 locus, thereby inhibiting expression of inserted coding sequences (e.g., encoding CISC components, heterologous TCRβ or TCRα chains, or FoxP3) under control of the endogenous promoter. In some embodiments, the STOP codon is in-frame with one or more upstream START codons, such that mRNA produced following transcription from the endogenous upstream promoter is not translated past the STOP codon.

### Chemically Induced Signaling Complex (CISC)

Embodiments of the systems for producing genetically modified cells described herein, each nucleic acid for insertion into the cell genome comprises a nucleotide sequence encoding a chemically induced signaling complex (CISC) component, each CISC component comprising an extracellular domain that binds rapamycin, a transmembrane domain, and an intracellular domain comprising or derived from an interleukin-2 receptor (IL-2R) cytoplasmic domain. In some embodiments, the first nucleic acid (for insertion into the TRAC locus) encodes a first CISC component comprising (i) an extracellular binding domain comprising an FK506-binding protein 12 (FKBP) domain, (ii) a transmembrane domain comprising or derived from an IL-2Rγ transmembrane domain, and (iii) an intracellular domain comprising or derived from an IL-2Rγ cytoplasmic domain; and the second nucleic acid (for insertion into the FOXP3 locus) encodes a first CISC component comprising (i) an extracellular binding domain comprising an FKBP-rapamycin-binding domain, (ii) a transmembrane domain comprising or derived from an IL-2Rβ transmembrane domain, and (iii) an intracellular domain comprising or derived from an IL-2Rβ cytoplasmic domain. A domain of a CISC component (e.g., transmembrane domain of the first CISC component) is “derived from” a given domain of an IL-2R polypeptide (e.g., IL-2Rγ) if it comprises at least 90% sequence identity to a wild-type (naturally occurring) amino acid sequence of the domain (e.g., a naturally occurring IL-2Rγ transmembrane domain).

Expression of CISC components in a cell allows selective induction of IL-2 signal transduction in a cell by manipulation of the presence and/or concentration of the rapamycin. Such controllable induction of signaling allows, for example, selective expansion of cells expressing both CISC components, where the IL-2 signal transduction event results in proliferation of the cell. In some embodiments, where two nucleic acids, each encoding a different CISC component, are introduced into the cell, such selective expansion allows for selection of cells that contain both nucleic acids, as contacting a cell comprising only one CISC component with rapamycin would not induce dimerization with the absent second CISC component, and thus not lead to IL-2 signal transduction.

Non-limiting examples of intracellular signaling domains include IL-2Rβ and IL-2Rγ cytoplasmic domains and functional derivatives thereof. In some embodiments, an intracellular signaling domain of the first CISC component comprises an IL-2Rγ domain or a functional derivative thereof, and an intracellular signaling domain of a second CISC component comprises an IL-2Rβ cytoplasmic domain or a functional derivative thereof. In some embodiments, dimerization of the first and second CISC components induces phosphorylation of JAK1, JAK3, and/or STAT5 in the cell. In some embodiments, dimerization of the first and second CISC components induces proliferation of the cell.

Non-limiting examples of transmembrane domains include IL-2Rβ and IL-2Rγ transmembrane domains and functional derivatives thereof. In some embodiments, the transmembrane domain of a CISC component is derived from the same protein as the intracellular signaling domain of the CISC component (e.g., a CISC component comprising an IL-2Rβ intracellular domain comprises an IL-2Rβ transmembrane domain). In some embodiments, one CISC component comprises an IL-2Rβ transmembrane domain, and the other CISC component comprises an IL-2Rγ transmembrane domain.

Non-limiting examples of extracellular binding domains capable of binding to rapamycin include an FK506-binding protein (FKBP) domain and an FKBP-rapamycin-binding (FRB) domain. FKBP and FRB domains are capable of binding to rapamycin or rapalogs, such as those described below, to form a heterodimer. In some embodiments, an extracellular binding domain of one CISC component comprises an FKBP domain, and an extracellular binding domain of the other CISC component comprises an FRB domain. In some embodiments, the CISC components form a heterodimer in the presence of rapamycin. In some embodiments, the FRB domain comprises a threonine at a position corresponding to amino acid 2098 of wild-type mTOR having the amino acid sequence of SEQ ID NO: 236. Mutation of this amino acid increases the affinity of mTOR for compounds having related structures to rapamycin, but decreases the affinity of mTOR for rapamycin itself. Thus, inclusion of a threonine at this position maintains the ability of mTOR to bind to rapamycin. The amino acid of a CISC component or FRB domain that “corresponds to” amino acid 2098 of wild-type mTOR may be determined by aligning a candidate sequence of a CISC component or FRB domain to SEQ ID NO: 236 (e.g., by BLAST or another alignment algorithm known in the art), with the amino acid aligned to amino acid 2098 of SEQ ID NO: 236 being the amino acid that “corresponds to” amino acid 2098 of SEQ ID NO: 236.

Each of the extracellular binding domains, transmembrane domains, and intracellular signaling domains of the CISC components described herein may be connected to another domain of the same CISC component by a linker. Linkers are known in the art. In some embodiments, the linker comprises 1, 2, 3, 4, 5, 6, 7, 8, 9 or 10 amino acids, such as glycines, or a number of amino acids, such as glycine, within a range defined by any two of the aforementioned numbers. In some embodiments, the glycine spacer comprises at least 3 glycines. In some embodiments, the glycine spacer comprises a sequence set forth as GSG, GGGS (SEQ ID NO: 229), GGGSGGG (SEQ ID NO: 230) or GGG. In some embodiments, the glycine spacer comprises the amino acid sequence GSG.

An extracellular binding domain may be connected to a transmembrane domain by a hinge domain. A hinge refers to a domain that links the extracellular binding domain to the transmembrane domain, and may confer flexibility to the extracellular binding domain. In some embodiments, the hinge domain positions the extracellular binding domain close to the plasma membrane to minimize the potential for recognition by antibodies or binding fragments thereof. In some embodiments, the extracellular binding domain is located N-terminal to the hinge domain. In some embodiments, the hinge domain may be natural or synthetic.

In some embodiments, the first and second CISC components form a heterodimer in the presence of rapamycin. In some embodiments, the first and second CISC components form a heterodimer in the presence of a compound that produced in vivo by metabolism of a rapalog. In some embodiments, the compound produced by in vivo metabolism of the rapalog is rapamycin. Non-limiting examples of rapalogs include everolimus, CCI-779, C20-methallylrapamycin, C16-(S)-3-methylindolerapamycin, C16-iRap, C16-(S)-7-methylindolerapamycin, AP21967, C16-(S)Butylsulfonamidorapamycin, AP23050, sodium mycophenolic acid, benidipine hydrochloride, AP1903, and AP23573, and metabolites or derivatives thereof.

In some embodiments, the nucleic acid encoding the second CISC component (FRB-IL2Rβ) further comprises a nucleotide sequence encoding a third CISC component that is capable of binding to rapamycin. Such CISC components are useful, for example, for binding to intracellular rapamycin, thereby preventing the bound rapamycin from interacting with other intracellular molecules or structures (e.g., preventing rapamycin from interacting with mTOR). In some embodiments, the third CISC component is a soluble protein that does not comprise a transmembrane domain. In some embodiments, the third CISC component comprises an intracellular FRB domain. In some embodiments, a third CISC component is a soluble protein comprising an FRB domain and lacking a transmembrane domain.

Nucleic acids encoding a first, second, and/or third CISC component may be comprised in one or more vectors. In some embodiments, a nucleic acid encoding a first CISC component is present on a separate vector from a nucleic acid encoding the second CISC component. In some embodiments, a nucleic acid encoding the third CISC component is present on the same vector as a nucleic acid encoding the second CISC component. In some embodiments, one or more vectors are viral vectors. In some embodiments, one or more vectors are adeno-associated viral (AAV) vectors. In some embodiments, one or more AAV vectors is an AAV1, AAV2, AAV3, AAV4, AAV5, AAV6, AAV7, AAV8, AAV9, AAV10, or AAV11 vector. In some embodiments, one or more AAV vectors are AAV5 vectors. In some embodiments, one or more AAV vectors are AAV6 vectors.

In some embodiments, a CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 66 or 71. In some embodiments, one or more CISC components further comprise a signal peptide. The signal peptide may be any signal peptide known in the art that directs the translated CISC component to the cell membrane. In some embodiments, each of the first and second CISC components comprises an LCN2 signal peptide. In some embodiments, each of the first and second CISC components comprises a signal peptide comprising the amino acid sequence of SEQ ID NO: 61

In some embodiments, one CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 66, and the other CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 71. In some embodiments, each CISC component further comprises a signal peptide, which may have the same or different amino acid sequences. The signal peptides may be any signal peptide known in the art that directs the translated CISC component to the cell membrane.

In some embodiments, a third CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 72. In some embodiments, a third CISC component consists of an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 72. In some embodiments, the third CISC component comprises the amino acid sequence of SEQ ID NO: 72. In some embodiments, the third CISC component consists of the amino acid sequence of SEQ ID NO: 72. In some embodiments, the third CISC component does not comprise a signal peptide. In some embodiments, the third CISC component does not comprise a transmembrane domain.

### T Cell Receptors (TCRs)

In some embodiments of the systems described herein, the TRAC locus of a cell is edited by inserting a nucleic acid comprising a promoter operably linked to a nucleotide sequence encoding a full-length TCRβ protein, and to a nucleotide sequence encoding at least a portion of a TCRα protein, such as TCRα variable and TCRα joining (TRAJ) regions that form the portion of a TCRα protein responsible for antigen-specificity. In some embodiments the nucleotide sequence encoding the TCRα variable and joining regions inserted in-frame with the endogenous nucleotide sequence encoding a portion of the TCRα constant domain, such that the inserted heterologous promoter initiates transcription of a sequence encoding a heterologous TCRβ protein and a sequence encoding a TCRα protein comprising heterologous TRAV/TRAJ amino acid sequences and an endogenous TCRα constant domain. This embodiment utilizes the endogenous 3′ regulatory region from the endogenous TRAC locus.

Genetically modified cells produced by systems described herein express a T cell receptor specific to a type 1 diabetes (T1D)-associated antigen. T cell receptors for expression by genetically modified cells are described herein under the heading “Methods for producing genetically modified cells” and subheading “T cell receptors (TCRs).” In certain embodiments, a nucleic acid encoding a TCR is codon-optimized to enhance expression in a particular host cell, e.g., a cell of the immune system, a hematopoietic stem cell, a T cell, a primary T cell, a T cell line, a NK cell, or a natural killer T cell. See, e.g., Scholten et al., C/in Immunol. 2006. 119:135.

In some embodiments, a nucleic acid described herein encodes a TCRβ chain and at least a portion of a TCRα chain that, expressed in combination, form a T1D2 TCR that binds to a peptide of IGRP(305-234). In other embodiments, a TCRβ chain and full-length TCRα chain, a portion of which is encoded by a nucleic acid described herein, form a T1D4 TCR that binds a peptide of IGRP(241-260). In other embodiments, a TCRβ chain and full-length TCRα chain, a portion of which is encoded by a nucleic acid described herein, form a T1D5-1 TCR that binds a peptide of IGRP(305-324). In some embodiments, the peptide of IGRP(305-324) is recognized when bound to HLA-DRB1*0401. In some embodiments, the peptide of IGRP(241-260) is recognized when bound to HLA-DRB1*0401.

In some embodiments, a TCR formed by a TCRβ chain and (at least a portion of) the TCRα chain encoded by a nucleic acid described herein comprises a TCRα variable (Vα) domain having three complementarity determining regions (CDRs) of αCDR1, αCDR2, and αCDR3; and a TCRβ variable (Vβ) domain having three CDRs of βCDR1, βCDR2, and βCDR3. Representative amino acids of CDRs of TCRs described herein are shown in Table 1, and nucleotide sequences encoding the same are shown in Table 2. In some embodiments: (i) αCDR1 comprises SEQ ID NO: 1, (ii) αCDR2 comprises SEQ ID NO: 2, (iii) αCDR3 comprises SEQ ID NO: 3, (iv) βCDR1 comprises SEQ ID NO: 4, (v) βCDR2 comprises SEQ ID NO: 5, and (vi) βCDR3 comprises SEQ ID NO: 6. In some embodiments: (i) αCDR1 comprises SEQ ID NO: 11, (ii) αCDR2 comprises SEQ ID NO: 12, (iii) αCDR3 comprises SEQ ID NO: 13, (iv) βCDR1 comprises SEQ ID NO: 14, (v) βCDR2 comprises SEQ ID NO: 15, and (vi) βCDR3 comprises SEQ ID NO: 16. In some embodiments: (i) αCDR1 comprises SEQ ID NO: 21, (ii) αCDR2 comprises SEQ ID NO: 22, (iii) αCDR3 comprises SEQ ID NO: 23, (iv) βCDR1 comprises SEQ ID NO: 24, (v) βCDR2 comprises SEQ ID NO: 25, and (vi) βCDR3 comprises SEQ ID NO: 26. In other embodiments, each of the set of αCDR1, αCDR2, αCDR3, βCDR1, βCDR2, and βCDR3 may have an amino acid sequence having at least 80%, at least 85%, at least 90%, at least 91%, at least 92%, at least 93%, at least 94%, at least 95%, at least 96%, at least 97%, at least 98%, or at least 99% sequence identity to the respective amino acid sequences in any of the aforementioned combinations of amino acid sequences.

In some embodiments, Vα comprises SEQ ID NO: 7 and Vβ comprises SEQ ID NO: 8. In some embodiments, Vα comprises SEQ ID NO: 17 and Vβ comprises SEQ ID NO: 18. In some embodiments, Vα comprises SEQ ID NO: 27 and Vβ comprises SEQ ID NO: 28. In other embodiments, each of the pair of Vα and Vβ may have an amino acid sequence having at least 80%, at least 85%, at least 90%, at least 91%, at least 92%, at least 93%, at least 94%, at least 95%, at least 96%, at least 97%, at least 98%, or at least 99% sequence identity to the respective amino acid sequence any of the aforementioned combinations of amino acid sequences.

In some embodiments, the TCRα chain comprises SEQ ID NO: 9 and the TCRβ chain comprises SEQ ID NO: 10. In some embodiments, the TCRα chain comprises SEQ ID NO: 19 and the TCRβ chain comprises SEQ ID NO: 20. In some embodiments, the TCRα chain comprises SEQ ID NO: 29 and the TCRβ chain comprises SEQ ID NO: 30. In other embodiments, each of the pair of TCRα and TCRβ chains may have an amino acid sequence having at least 80%, at least 85%, at least 90%, at least 91%, at least 92%, at least 93%, at least 94%, at least 95%, at least 96%, at least 97%, at least 98%, or at least 99% sequence identity to the respective amino acid sequence of any of the aforementioned combinations of amino acid sequences.

### FOXP3 Locus Modification

In some embodiments of the system described herein, a nucleic acid for targeted insertion into the FOXP3 locus comprises a promoter that, following insertion, becomes operably linked to a nucleotide sequence encoding a portion of the endogenous FoxP3 protein. The inserted promoter is introduced into the genome downstream from the Treg-specific demethylated region (TSDR) of the FOXP3 locus. In unmodified cells, the TSDR epigenetically regulates expression of FoxP3, inhibiting FoxP3 production in cells exposed to inflammatory conditions, which may result in loss of FoxP3 expression and conversion of unmodified Treg cells to a T effector (Teff) phenotype. Insertion of a promoter downstream from the TSDR bypasses TSDR-mediated regulation of FOXP3 expression, thereby providing stable production of FoxP3 even in inflammatory conditions.

The heterologous promoter may be inserted at any position downstream from the endogenous promoter (e.g., downstream from the TSDR) and upstream from or within the first coding exon of the FOXP3 coding sequence. This first coding exon is known in the art as exon 2, as it is the second exon present in pre-mRNA transcribed from the endogenous FOXP3 promoter, and the first coding exon because it is this exon, not exon 1 (the first exon of FOXP3-encoding pre-mRNA) that contains the start codon that initiates translation of wild-type FoxP3. In some embodiments, the heterologous promoter is inserted 1-10,000, 10-1,000, 10-100, 10-5,000, 20-4,000, 30-3,000, 40-2,000, 50-1,000, 60-750, 70-500, 80-400, 90-300, 100-200, 1-1,000, 1,000-2,000, 2,000-3,000, 3,000-4,000, 4,000-5,000, 5,000-6,000, 6,000-7,000, 7,000-8,000, 8,000-9,000, or 9,000-10,000 nucleotides downstream from the TSDR of FOXP3. In some embodiments, the heterologous promoter is inserted 1-10,000, 10-1,000, 10-100, 10-5,000, 20-4,000, 30-3,000, 40-2,000, 50-1,000, 60-750, 70-500, 80-400, 90-300, 100-200, 1-1,000, 1,000-2,000, 2,000-3,000, 3,000-4,000, 4,000-5,000, 5,000-6,000, 6,000-7,000, 7,000-8,000, 8,000-9,000, or 9,000-10,000 nucleotides upstream from the first coding exon of the FOXP3 coding sequence. In some embodiments, the heterologous promoter is inserted into the first coding exon, such that a synthetic first coding exon is created, where the synthetic first coding exon differs from the endogenous first coding exon but still comprises a start codon that is in-frame with the FOXP3 coding sequence of downstream FOXP3 exons.

### 2A Motifs and Linkers

Some embodiments of nucleic acids described herein encoding multiple polypeptides or portions thereof may contain intervening nucleotide sequences encoding a 2A motifs. 2A motifs are known in the art, and are useful for promoting production of multiple polypeptides from translation of a single nucleotide sequence. See, e.g., Kim et al., PLoS ONE. 2011. 6:e18556. In some embodiments, the 2A motif is translated, and self-cleavage of the polypeptide occurs following translation, resulting in release of separate polypeptides. In other embodiments, the nucleotide sequence encoding the 2A motif causes the ribosome to progress along an mRNA without incorporating an encoded amino acid of the 2A motif, resulting in release of the first polypeptide (e.g., first FKBP-IL2Rγ CISC component), and allowing translation initiation of a second polypeptide (e.g., TCRβ chain).

In some embodiments, nucleotide sequences encoding a 2A motif are present in-frame with and between each pair of nucleotide sequences encoding (i) the first (FKBP-IL2Rγ) CISC component; (ii) the TCRβ chain; and (iii) the TCRα chain or portion thereof. Thus, the heterologous promoter (e.g., MND promoter) initiates transcription of a single mRNA encoding each of the CISC component, TCRβ chain, and TCRα chain, with intervening 2A motifs allowing production of each as a separate polypeptide. In some embodiments, a nucleotide sequence encoding a 2A motif is in-frame with and between each pair of nucleotide sequences encoding (i) the second (FKBP-IL2Rγ) CISC component; (ii) the cytosolic FRB domain; and (iii) FoxP3. Thus, the heterologous promoter (e.g., MND promoter) initiates transcription of a single mRNA encoding each of the CISC component, cytosolic FRB domain, and FoxP3, with intervening 2A motifs allowing production of each as a separate polypeptide.

The 2A motifs encoded by nucleotide sequences between each pair of sequences encoding two polypeptides (e.g., sequences encoding an FKBP-IL2Rγ CISC component and TCRβ chain; TCRβ chain and portion of α chain) may be any 2A motif known in the art. In some embodiments, the encoded 2A motifs between each pair of nucleotide sequences encoding distinct polypeptides may be independently selected from the group consisting of F2A, P2A, T2A, E2A. In some embodiments, a first encoded 2A motif and second encoded 2A motif on a nucleic acid are different 2A motifs. Use of different 2A motifs in the same inserted nucleic acid reduces the probability of internal recombination, which may result in the nucleotide sequence between the recombined 2A motifs being excised from the chromosome. In some embodiments, a nucleotide sequence encoding a first 2A motif has no more than 90% sequence identity to a nucleotide sequence encoding a second 2A motif on the same nucleic acid. In some embodiments, a nucleotide sequence encoding a first 2A motif has no more than 80% sequence identity to a nucleotide sequence encoding a second 2A motif on the same nucleic acid. In some embodiments, a nucleotide sequence encoding a first 2A motif has no more than 70% sequence identity to a nucleotide sequence encoding a second 2A motif on the same nucleic acid. In some embodiments, a nucleotide sequence encoding a first 2A motif has no more than 60% sequence identity to a nucleotide sequence encoding a second 2A motif on the same nucleic acid. In some embodiments, a nucleotide sequence encoding a first 2A motif has no more than 50% sequence identity to a nucleotide sequence encoding a second 2A motif on the same nucleic acid. In some embodiments, a first 2A motif is a T2A motif, and the second motif is a P2A motif.

In other embodiments, the first and second 2A motifs encoded by nucleotide sequences on the nucleic acid are the same 2A motif. In some embodiments, a nucleic acid comprises a nucleotide sequence encoding a first P2A motif, and a second nucleotide sequence encoding a second P2A motif, with the nucleotide sequence encoding the first P2A motif comprising at least 80% sequence identity to the nucleotide sequence encoding the second P2A motif. In some embodiments, the first and second nucleotide sequences encoding the first and second P2A motifs comprise the same nucleotide sequences.

In some embodiments, the nucleic acid for insertion into the TRAC locus comprises: (i) a sequence encoding a T2A motif between the sequence encoding the first CISC component and the sequence encoding the TCRβ chain; and (ii) a sequence encoding a P2A motif between the sequence encoding the TCRβ chain and heterologous TCRα chain portion.

In some embodiments, the nucleic acid for insertion into the FOXP3 locus comprises: (i) a sequence encoding a P2A motif between the sequence encoding the second CISC component and the sequence encoding the cytosolic FRB domain; and (ii) a second sequence encoding a second P2A motif between the sequence encoding the cytosolic FRB domain and the sequence encoding FoxP3.

In some embodiments, a polypeptide (e.g., CISC components and/or TCRβ chains) encoded by a nucleic acid for insertion into the cell genome comprises a C-terminal linker. Incorporation of such a linker may, for example, improve efficiency of cleavage in 2A motifs and/or prevent cleavage of a 2A motif from excising amino acids of the encoded CISC component or TCRβ chain. In some embodiments, the encoded first CISC component comprises a C-terminal linker. In some embodiments, the encoded second CISC component comprises a C-terminal linker. In some embodiments, the encoded cytosolic FRB domain component comprises a C-terminal linker. In some embodiments, the encoded TCRβ chain comprises a C-terminal linker.

Linkers at the C-terminus of encoded polypeptides may be any linker known in the art. In some embodiments, the linker comprises 1, 2, 3, 4, 5, 6, 7, 8, 9 or 10 amino acids, such as glycines, or a number of amino acids, such as glycine, within a range defined by any two of the aforementioned numbers. In some embodiments, the linker comprises at least 3 glycines. In some embodiments, the linker comprises a sequence set forth as GSG, GGGS (SEQ ID NO: 229), GGGSGGG (SEQ ID NO: 230) or GGG. In some embodiments, the linker comprises the amino acid sequence GSG. In some embodiments, each of the first CISC component, second CISC component, cytosolic FRB domain, and TCRβ chain comprises a C-terminal linker having the amino acid sequence GSG.

### Vectors

The first and/or second nucleic acids for insertion into the TRAC and FOXP3 loci, respectively, may be comprised in one or more vectors. In some embodiments, the first TRAC locus-targeting nucleic acid is comprised in a first vector, and the FOXP3 locus-targeting nucleic acid is comprised in a second vector. In some cases, the vector is packaged in a virus capable of infecting the cell (e.g., the vector is a viral vector). Exemplary viruses include adenovirus, retrovirus, lentivirus, adeno-associated virus, and others that are known in the art and disclosed herein.

The term “vector” is used to refer to any molecule (e.g., nucleic acid, plasmid) or arrangement of molecules (e.g., virus) used to transfer coding information to a host cell. The term “expression vector” refers to a vector that is suitable for introduction of a host cell and contains nucleic acid sequences that direct and/or control expression of introduced heterologous nucleic acid sequences. Expression includes, but is not limited to, processes such as transcription, translation, and RNA splicing, if introns are present. Non-limiting examples of vectors include artificial chromosomes, minigenes, cosmids, plasmids, phagemids, and viral vectors. Non-limiting examples of viral vectors include lentiviral vectors, retroviral vectors, herpesvirus vectors, adenovirus vectors, and adeno-associated viral vectors. In some embodiments, one or more vectors comprising nucleic acids for use in the systems provided herein are lentiviral vectors. In some embodiments, one or more vectors are adenoviral vectors. In some embodiments, one or more vectors are adeno-associated viral (AAV) vectors. In some embodiments, one or more AAV vectors is an AAV1, AAV2, AAV3, AAV4, AAV5, AAV6, AAV7, AAV8, AAV9, AAV10, or AAV 11 vector. In some embodiments, a vector comprising the nucleic acid for insertion into the TRAC locus is an AAV1, AAV2, AAV3, AAV4, AAV5, AAV6, AAV7, AAV8, AAV9, AAV10, or AAV11 vector. In some embodiments, a vector comprising the nucleic acid for insertion into the FOXP3 locus is an AAV1, AAV2, AAV3, AAV4, AAV5, AAV6, AAV7, AAV8, AAV9, AAV10, or AAV11 vector.

In some embodiments, one or more AAV vectors are AAV5 vectors. In some embodiments, one or more AAV vectors are AAV6 vectors. In some embodiments, both the first and second nucleic acids are comprised in separate AAV5 vectors. In some embodiments, both the first and second nucleic acids are comprised in separate AAV6 vectors.

In some embodiments, a nucleic acid for insertion into the TRAC locus comprises, between the 5′ and 3′ homology arms, a nucleotide sequence having at least 90% sequence identity to any one of SEQ ID NOs: 94, 106, 117, 128, and 139. In some embodiments, the nucleotide sequence comprises at least 95% sequence identity to any one of SEQ ID NOS: 94, 106, 117, 128, and 139. In some embodiments, the nucleotide sequence comprises any one of SEQ ID NOS: 94, 106, 117, 128, and 139. In some embodiments, the nucleic acid comprises the nucleotide sequence of SEQ ID NO: 94. In some embodiments, the nucleic acid comprises the nucleotide sequence of SEQ ID NO: 106. In some embodiments, the nucleic acid comprises the nucleotide sequence of SEQ ID NO: 117. In some embodiments, the nucleic acid comprises the nucleotide sequence of SEQ ID NO: 128. In some embodiments, the nucleic acid comprises the nucleotide sequence of SEQ ID NO: 139.

In some embodiments, a nucleic acid for insertion into the TRAC locus comprises at least 90% sequence identity to the nucleotide sequence of any one of SEQ ID NOs: 95, 107, 118, 129, and 140. In some embodiments, the nucleic acid comprises at least 95% sequence identity to the nucleotide sequence of any one of SEQ ID NOs: 95, 107, 118, 129, and 140. In some embodiments, the nucleic acid comprises the nucleotide sequence of any one of SEQ ID NOs: 95, 107, 118, 129, and 140. In some embodiments, the nucleic acid comprises at least 95% sequence identity to the nucleotide sequence of SEQ ID NO: 95. In some embodiments, the nucleic acid comprises at least 95% sequence identity to the nucleotide sequence of SEQ ID NO: 107. In some embodiments, the nucleic acid comprises at least 95% sequence identity to the nucleotide sequence of SEQ ID NO: 118. In some embodiments, the nucleic acid comprises at least 95% sequence identity to the nucleotide sequence of SEQ ID NO: 129. In some embodiments, the nucleic acid comprises at least 95% sequence identity to the nucleotide sequence of SEQ ID NO: 140. In some embodiments, the nucleic acid comprises the nucleotide sequence of SEQ ID NO: 95. In some embodiments, the nucleic acid comprises the nucleotide sequence of SEQ ID NO: 107. In some embodiments, the nucleic acid comprises the nucleotide sequence of SEQ ID NO: 118. In some embodiments, the nucleic acid comprises the nucleotide sequence of SEQ ID NO: 129. In some embodiments, the nucleic acid comprises the nucleotide sequence of SEQ ID NO: 140.

In some embodiments, a nucleic acid for insertion into the FOXP3 locus comprises, between the 5′ and 3′ homology arms, a nucleotide sequence having at least 90% sequence identity to any one of SEQ ID NOs: 150, 161, 172, 184, 195, 206, and 218. In some embodiments, the nucleotide sequence comprises at least 95% sequence identity to any one of SEQ ID NOS: 150, 161, 172, 184, 195, 206, and 218. In some embodiments, the nucleotide sequence comprises any one of SEQ ID NOS: 150, 161, 172, 184, 195, 206, and 218. In some embodiments, the nucleic acid comprises the nucleotide sequence of SEQ ID NO: 150. In some embodiments, the nucleic acid comprises the nucleotide sequence of SEQ ID NO: 161. In some embodiments, the nucleic acid comprises the nucleotide sequence of SEQ ID NO: 172. In some embodiments, the nucleic acid comprises the nucleotide sequence of SEQ ID NO: 184. In some embodiments, the nucleic acid comprises the nucleotide sequence of SEQ ID NO: 195. In some embodiments, the nucleic acid comprises the nucleotide sequence of SEQ ID NO: 206. In some embodiments, the nucleic acid comprises the nucleotide sequence of SEQ ID NO: 218.

In some embodiments, a nucleic acid for insertion into the FOXP3 locus comprises at least 90% sequence identity to the nucleotide sequence of any one of SEQ ID NOs: 151, 162, 173, 185, 196, 207, and 219. In some embodiments, the nucleic acid comprises at least 95% sequence identity to the nucleotide sequence of any one of SEQ ID NOs: 151, 162, 173, 185, 196, 207, and 219. In some embodiments, the nucleic acid comprises the nucleotide sequence of any one of SEQ ID NOs: 151, 162, 173, 185, 196, 207, and 219. In some embodiments, the nucleic acid comprises at least 95% sequence identity to the nucleotide sequence of SEQ ID NO: 151. In some embodiments, the nucleic acid comprises at least 95% sequence identity to the nucleotide sequence of SEQ ID NO: 162. In some embodiments, the nucleic acid comprises at least 95% sequence identity to the nucleotide sequence of SEQ ID NO: 173. In some embodiments, the nucleic acid comprises at least 95% sequence identity to the nucleotide sequence of SEQ ID NO: 185. In some embodiments, the nucleic acid comprises at least 95% sequence identity to the nucleotide sequence of SEQ ID NO: 196. In some embodiments, the nucleic acid comprises at least 95% sequence identity to the nucleotide sequence of SEQ ID NO: 207. In some embodiments, the nucleic acid comprises at least 95% sequence identity to the nucleotide sequence of SEQ ID NO: 219. In some embodiments, the nucleic acid comprises the nucleotide sequence of SEQ ID NO: 151. In some embodiments, the nucleic acid comprises the nucleotide sequence of SEQ ID NO: 162. In some embodiments, the nucleic acid comprises the nucleotide sequence of SEQ ID NO: 173. In some embodiments, the nucleic acid comprises the nucleotide sequence of SEQ ID NO: 185. In some embodiments, the nucleic acid comprises the nucleotide sequence of SEQ ID NO: 196. In some embodiments, the nucleic acid comprises the nucleotide sequence of SEQ ID NO: 207. In some embodiments, the nucleic acid comprises the nucleotide sequence of SEQ ID NO: 219.

### Homology Arms

Nucleic acids for insertion into TRAC or FOXP3 loci using the systems described herein comprise 5′ and 3′ homology arms, to target insertion of the nucleic acid into the TRAC or FOXP3 locus, respectively, by homology-directed repair following introduction of a double-stranded break. Typically, the 5′ homology arm refers to a homology arm at the 5′ end of the nucleic acid, and 3′ homology arm refers to another homology arm at the 3′ end of the nucleic acid, when considering the coding strand of the nucleic acid (i.e., the strand containing the reading frame(s) encoding polypeptides including CISC components, TCR chains, and FoxP3). The 5′ homology arm will have homology to a first sequence in the targeted locus, and the 3′ homology arm will have homology to a second sequence in the targeted locus that is downstream from the first sequence in the targeted locus, such that the nucleic acid is inserted into the locus in a targeted manner. Following insertion, the modified locus will comprise the homology arms, in place of the first and second sequences in the targeted locus, and the sequence between the homology arms on the nucleic acid, in place of the sequence that was previously present between the first and second sequences in the targeted locus. The homology arms may be the same length, have similar lengths (within 100 bp of each other), or different lengths. In some embodiments, one or both homology arms have a length of 100-2,000 bp, 400-1,500 bp, 500-1,000 bp. In some embodiments, one or both homology arms are about 100 bp, about 200 bp, about 300 bp, about 400 bp, about 500 bp, about 600 bp, about 700 bp, about 800 bp, about 900 bp, about 1,000 bp, about 1,100 bp, about 1,200 bp, about 1,300 bp, about 1,400 bp, about 1,500 bp, about 1,600 bp, about 1,700 bp, about 1,800 bp, about 1,900 bp, or about 2,000 bp. In some embodiments, both homology arms are 100-2,000 nucleotides in length. In some embodiments, both homology arms are 300-1,000 nucleotides in length. In some embodiments, both homology arms are 300-700 nucleotides in length. In some embodiments, both homology arms are 300-500 nucleotides in length. In some embodiments, both homology arms are 500-700 nucleotides in length. In some embodiments, both homology arms are 700-1,000 nucleotides in length.

Homology arms of a nucleic acid for insertion at a targeted genomic locus may be chosen based on homologous sequences in the targeted locus that are upstream and/or downstream from a site targeted for cleavage by a nuclease. For example, in some embodiments for insertion by homology-directed repair following cleavage at a given position (cleavage site) in the targeted locus, the 5′ homology arm of a nucleic acid for insertion has homology to a sequence upstream of the cleavage site, and the 3′ homology arm of the nucleic acid has homology to a sequence downstream of the cleavage site. In some embodiments, the 5′ homology arm has homology to a sequence 100-2,000 nucleotides in length that ends 25-5,000, 50-3,000, 75-2,000, 100-1,000, 150-500 nucleotides upstream from the cleavage site. In some embodiments, the 5′ homology arm has homology to a sequence 100-2,000 nucleotides in length that ends 25-5,000, 50-3,000, 75-2,000, 100-1,000, 150-500 nucleotides upstream from a PAM sequence cleaved by an RNA-guided nuclease. In some embodiments, the 5′ homology arm has homology to a sequence 100-2,000 nucleotides in length that ends 25-5,000, 50-3,000, 75-2,000, 100-1,000, 150-500 nucleotides upstream from a sequence in the genome that is complementary to a spacer sequence of a gRNA. In some embodiments, the 5′ homology arm has homology to a sequence 100-2,000 nucleotides in length that ends at a position 150-500 nucleotides upstream from a cleavage site. In some embodiments, the 5′ homology arm has homology to a sequence 100-2,000 nucleotides in length that ends at a position 150-500 nucleotides upstream from a PAM sequence cleaved by an RNA-guided nuclease. In some embodiments, the 5′ homology arm has homology to a sequence 100-2,000 nucleotides in length that ends at a position 150-500 nucleotides upstream from a sequence in the genome that is complementary to a spacer sequence of a gRNA.

In some embodiments, the 3′ homology arm has homology to a sequence 100-2,000 nucleotides in length that ends 25-5,000, 50-3,000, 75-2,000, 100-1,000, 150-500 nucleotides upstream from the cleavage site. In some embodiments, the 3′ homology arm has homology to a sequence 100-2,000 nucleotides in length that ends 25-5,000, 50-3,000, 75-2,000, 100-1,000, 150-500 nucleotides upstream from a PAM sequence cleaved by an RNA-guided nuclease. In some embodiments, the 3′ homology arm has homology to a sequence 100-2,000 nucleotides in length that ends 25-5,000, 50-3,000, 75-2,000, 100-1,000, 150-500 nucleotides upstream from a sequence in the genome that is complementary to a spacer sequence of a gRNA. In some embodiments, the 3′ homology arm has homology to a sequence 100-2,000 nucleotides in length that ends at a position 150-500 nucleotides upstream from a cleavage site. In some embodiments, the 3′ homology arm has homology to a sequence 100-2,000 nucleotides in length that ends at a position 150-500 nucleotides upstream from a PAM sequence cleaved by an RNA-guided nuclease. In some embodiments, the 3′ homology arm has homology to a sequence 100-2,000 nucleotides in length that ends at a position 150-500 nucleotides upstream from a sequence in the genome that is complementary to a spacer sequence of a gRNA.

In some embodiments, where a system includes a gRNA comprising a spacer sequence, neither the 5′ nor the 3′ homology arm of a nucleic acid for genomic insertion comprises a sequence that is complementary to the spacer sequence. In such embodiments, lack of a complementary sequence on the donor template reduces the chance of the gRNA binding to the donor template and mediating cleavage, which can reduce the efficiency of genomic insertion. In some embodiments, the donor template does not comprise a sequence that is complementary to the spacer sequence. In embodiments where a different nuclease that does not require a gRNA for targeted cleavage is used, the donor template does not comprise a sequence that is cleaved by the nuclease.

In some embodiments, a nucleic acid for insertion into the TRAC locus comprises a 5′ homology arm with at least 90% sequence identity to the nucleotide sequence of SEQ ID NO: 85, and a 3′ homology arm with at least 90% sequence identity to the nucleotide sequence of SEQ ID NO: 93. In some embodiments, the 5′ homology arm comprises at least 95% sequence identity to the nucleotide sequence of SEQ ID NO: 85, and the 3′ homology arm comprises at least 95% to the nucleotide sequence of SEQ ID NO: 93. In some embodiments, the 5′ homology arm comprises the nucleotide sequence of SEQ ID NO: 85, and the 3′ homology arm comprises the nucleotide sequence of SEQ ID NO: 93.

In some embodiments, a nucleic acid for insertion into the TRAC locus comprises a 5′ homology arm with at least 90% sequence identity to the nucleotide sequence of SEQ ID NO: 96, and a 3′ homology arm with at least 90% sequence identity to the nucleotide sequence of SEQ ID NO: 105. In some embodiments, the 5′ homology arm comprises at least 95% sequence identity to the nucleotide sequence of SEQ ID NO: 96, and the 3′ homology arm comprises at least 95% to the nucleotide sequence of SEQ ID NO: 105. In some embodiments, the 5′ homology arm comprises the nucleotide sequence of SEQ ID NO: 96, and the 3′ homology arm comprises the nucleotide sequence of SEQ ID NO: 105.

In some embodiments, a nucleic acid for insertion into the TRAC locus comprises a 5′ homology arm with at least 90% sequence identity to the nucleotide sequence of SEQ ID NO: 108, and a 3′ homology arm with at least 90% sequence identity to the nucleotide sequence of SEQ ID NO: 116. In some embodiments, the 5′ homology arm comprises at least 95% sequence identity to the nucleotide sequence of SEQ ID NO: 108, and the 3′ homology arm comprises at least 95% to the nucleotide sequence of SEQ ID NO: 116. In some embodiments, the 5′ homology arm comprises the nucleotide sequence of SEQ ID NO: 108, and the 3′ homology arm comprises the nucleotide sequence of SEQ ID NO: 116.

In some embodiments, a nucleic acid for insertion into the TRAC locus comprises a 5′ homology arm with at least 90% sequence identity to the nucleotide sequence of SEQ ID NO: 119, and a 3′ homology arm with at least 90% sequence identity to the nucleotide sequence of SEQ ID NO: 127. In some embodiments, the 5′ homology arm comprises at least 95% sequence identity to the nucleotide sequence of SEQ ID NO: 119, and the 3′ homology arm comprises at least 95% to the nucleotide sequence of SEQ ID NO: 127. In some embodiments, the 5′ homology arm comprises the nucleotide sequence of SEQ ID NO: 119, and the 3′ homology arm comprises the nucleotide sequence of SEQ ID NO: 127.

In some embodiments, a nucleic acid for insertion into the TRAC locus comprises a 5′ homology arm with at least 90% sequence identity to the nucleotide sequence of SEQ ID NO: 130, and a 3′ homology arm with at least 90% sequence identity to the nucleotide sequence of SEQ ID NO: 138. In some embodiments, the 5′ homology arm comprises at least 95% sequence identity to the nucleotide sequence of SEQ ID NO: 130, and the 3′ homology arm comprises at least 95% to the nucleotide sequence of SEQ ID NO: 138. In some embodiments, the 5′ homology arm comprises the nucleotide sequence of SEQ ID NO: 130, and the 3′ homology arm comprises the nucleotide sequence of SEQ ID NO: 138.

In some embodiments, a nucleic acid for insertion into the FOXP3 locus comprises a 5′ homology arm with at least 90% sequence identity to the nucleotide sequence of SEQ ID NO: 141, and a 3′ homology arm with at least 90% sequence identity to the nucleotide sequence of SEQ ID NO: 149. In some embodiments, the 5′ homology arm comprises at least 95% sequence identity to the nucleotide sequence of SEQ ID NO: 141, and the 3′ homology arm comprises at least 95% to the nucleotide sequence of SEQ ID NO: 149. In some embodiments, the 5′ homology arm comprises the nucleotide sequence of SEQ ID NO: 141, and the 3′ homology arm comprises the nucleotide sequence of SEQ ID NO: 149.

In some embodiments, a nucleic acid for insertion into the FOXP3 locus comprises a 5′ homology arm with at least 90% sequence identity to the nucleotide sequence of SEQ ID NO: 152, and a 3′ homology arm with at least 90% sequence identity to the nucleotide sequence of SEQ ID NO: 160. In some embodiments, the 5′ homology arm comprises at least 95% sequence identity to the nucleotide sequence of SEQ ID NO: 152, and the 3′ homology arm comprises at least 95% to the nucleotide sequence of SEQ ID NO: 160. In some embodiments, the 5′ homology arm comprises the nucleotide sequence of SEQ ID NO: 152, and the 3′ homology arm comprises the nucleotide sequence of SEQ ID NO: 160.

In some embodiments, a nucleic acid for insertion into the FOXP3 locus comprises a 5′ homology arm with at least 90% sequence identity to the nucleotide sequence of SEQ ID NO: 163, and a 3′ homology arm with at least 90% sequence identity to the nucleotide sequence of SEQ ID NO: 171. In some embodiments, the 5′ homology arm comprises at least 95% sequence identity to the nucleotide sequence of SEQ ID NO: 163, and the 3′ homology arm comprises at least 95% to the nucleotide sequence of SEQ ID NO: 171. In some embodiments, the 5′ homology arm comprises the nucleotide sequence of SEQ ID NO: 163, and the 3′ homology arm comprises the nucleotide sequence of SEQ ID NO: 171.

In some embodiments, a nucleic acid for insertion into the FOXP3 locus comprises a 5′ homology arm with at least 90% sequence identity to the nucleotide sequence of SEQ ID NO: 174, and a 3′ homology arm with at least 90% sequence identity to the nucleotide sequence of SEQ ID NO: 183. In some embodiments, the 5′ homology arm comprises at least 95% sequence identity to the nucleotide sequence of SEQ ID NO: 174, and the 3′ homology arm comprises at least 95% to the nucleotide sequence of SEQ ID NO: 183. In some embodiments, the 5′ homology arm comprises the nucleotide sequence of SEQ ID NO: 174, and the 3′ homology arm comprises the nucleotide sequence of SEQ ID NO: 183.

In some embodiments, a nucleic acid for insertion into the FOXP3 locus comprises a 5′ homology arm with at least 90% sequence identity to the nucleotide sequence of SEQ ID NO: 186, and a 3′ homology arm with at least 90% sequence identity to the nucleotide sequence of SEQ ID NO: 194. In some embodiments, the 5′ homology arm comprises at least 95% sequence identity to the nucleotide sequence of SEQ ID NO: 186, and the 3′ homology arm comprises at least 95% to the nucleotide sequence of SEQ ID NO: 194. In some embodiments, the 5′ homology arm comprises the nucleotide sequence of SEQ ID NO: 186, and the 3′ homology arm comprises the nucleotide sequence of SEQ ID NO: 194.

In some embodiments, a nucleic acid for insertion into the FOXP3 locus comprises a 5′ homology arm with at least 90% sequence identity to the nucleotide sequence of SEQ ID NO: 197, and a 3′ homology arm with at least 90% sequence identity to the nucleotide sequence of SEQ ID NO: 205. In some embodiments, the 5′ homology arm comprises at least 95% sequence identity to the nucleotide sequence of SEQ ID NO: 197, and the 3′ homology arm comprises at least 95% to the nucleotide sequence of SEQ ID NO: 205. In some embodiments, the 5′ homology arm comprises the nucleotide sequence of SEQ ID NO: 197, and the 3′ homology arm comprises the nucleotide sequence of SEQ ID NO: 205.

In some embodiments, a nucleic acid for insertion into the FOXP3 locus comprises a 5′ homology arm with at least 90% sequence identity to the nucleotide sequence of SEQ ID NO: 208, and a 3′ homology arm with at least 90% sequence identity to the nucleotide sequence of SEQ ID NO: 217. In some embodiments, the 5′ homology arm comprises at least 95% sequence identity to the nucleotide sequence of SEQ ID NO: 208, and the 3′ homology arm comprises at least 95% to the nucleotide sequence of SEQ ID NO: 217. In some embodiments, the 5′ homology arm comprises the nucleotide sequence of SEQ ID NO: 208, and the 3′ homology arm comprises the nucleotide sequence of SEQ ID NO: 217.

### Nucleases and Guide RNAs

Some aspects of the disclosure relate to the use of nucleases to introduce a double-stranded break into nucleic acid of a cell genome and edit the genome at a desired locus (e.g., to promote insertion of a donor template at the locus by homology-directed repair). Any one of multiple gene- or genome-editing methods or systems can used to accomplish editing of one or more loci (e.g., TRAC and/or FOXP3). Non-limiting examples of gene editing methods include use of a DNA endonuclease such as an RNA-guided nuclease (e.g., Cas (e.g., Cas9) nuclease), zinc finger nuclease (ZFN), transcription activator-like effector nuclease (TALEN), or meganuclease; transposon-mediated gene editing; serine integrase-mediated gene editing; and lentivirus-mediated gene editing.

In certain embodiments, a chromosomal gene knock-out or gene knock-in (e.g., insertion) is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. A DNA endonuclease refers to an endonuclease that is capable of catalyzing cleavage of a phosphodiester bond within a DNA polynucleotide. In some embodiments, an endonuclease is capable of cleaving a nucleic acid sequence in a targeted locus, promoting insertion of an exogenous nucleic acid sequence into the targeted locus by homologous recombination. An endonuclease may be a naturally occurring, recombinant, genetically modified, or fusion endonuclease. Examples of endonucleases for use in gene editing include zinc finger nucleases (ZFN), TALE-nucleases (TALEN), RNA-guided nucleases, CRISPR-Cas nucleases, meganucleases, and megaTALs.

The nucleic acid strand breaks caused by DNA endonucleases are typically double-strand breaks (DSB), which may be commonly repaired through the distinct mechanisms of homology directed repair (HDR) by homologous recombination, or by non-homologous end joining (NHEJ). (NHEJ: Ghezraoui et al., 2014 Mol Cell 55(6):829-842; HDR: Jasin and Rothstein, 2013 Cold Spring Harb Perspect Biol 5(11):a012740, PMID 24097900). During HDR/homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. HDR is favored by the presence of a donor template at the time of DSB formation.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted insertion of a transgene comprising flanking sequences homologous to the genome at the site of DSB is facilitated by homology directed repair (HDR). Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout or inactivation comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histidine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair (HDR) can introduce a transgene at the site of DSB providing homologous flanking sequences are present in the donor template containing the transgene. In certain embodiments, a gene knockout comprises an insertion, a deletion, a mutation or a combination thereof, and made using a TALEN molecule.

Gene-editing systems and methods described herein may make use of viral or non-viral vectors or cassettes, as well as nucleases that allow site-specific or locus-specific gene-editing, such as RNA-guided nucleases, Cas nucleases (e.g., Cpf1 or Cas9 nucleases), meganucleases, TALENs, or ZFNs. Certain RNA-guided nucleases useful with some embodiments provided herein are disclosed in U.S. Pat. No. 11,162,114, which is expressly incorporated by reference herein in its entirety. Non-limiting examples of Cas nucleases include SpCas9, SaCas9, CjCas9, xCas9, C2c1, Cas13a/C2c2, C2c3, Cas13b, Cpf1, and variants thereof. Certain features useful with some embodiments provided herein are disclosed in WO 2019/210057, which is expressly incorporated by reference in its entirety.

As used herein, a “clustered regularly interspaced short palindromic repeats/Cas” (CRISPR/Cas, or Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into types (e.g., type I, type II, type III, and type V) based on the sequence and structure of the Cas nucleases. The crRNA-guided surveillance complexes in types I and III need multiple Cas subunits. The Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a donor template transgene with homologous flanking sequences can be introduced at the site of DSB via homology directed repair (HDR). The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). Non-limiting examples of CRISPR/Cas nucleases include Cas9, SaCas9, CjCas9, xCas9, C2C1, Cas13a/C2c2, C2c3, Cas13b, Cpf1, and variants thereof. Other RNA-guided nucleases capable of introducing a double-stranded break in DNA in the presence of a guide RNA comprising a spacer sequence complementary to a target sequence of the DNA, by cleaving at a PAM sequence adjacent to the target sequence on the DNA, may also be used in gene editing methods and systems described herein. In some embodiments, the RNA-guided nuclease is a nuclease having (i.e., cleaving dsDNA at) a protospacer-adjacent motif (PAM) sequence of 5′-NNNNCC-3′. Exemplary RNA-guided nucleases having a PAM sequence of NNNNCC are described, e.g., in International Application No. PCT/US2019/035373, published as PCT Publication No. WO 2019/236566, which is incorporated by reference herein in its entirety. In some embodiments, the RNA-guided nuclease cleaves DNA at a PAM sequence of NGG, and localizes to DNA at a target sequence in the presence of a gRNA having the nucleotide sequence of SEQ ID NO: (SEQ ID NO: 237), where the polyN stretch of SEQ ID NO: 237 is the protospacer sequence complementary to the target DNA sequence. In some embodiments, the RNA-guided nuclease cleaves DNA at a PAM sequence of NNNNCC, and localizes to DNA at a target sequence in the presence of a gRNA having the nucleotide sequence of SEQ ID NO: 238, where the polyN stretch of SEQ ID NO: 238 is the protospacer sequence complementary to the target DNA sequence. In some embodiments, the RNA-guided nuclease cleaves DNA at a PAM sequence of NNNNCC, and localizes to DNA at a target sequence in the presence of a gRNA having the nucleotide sequence of SEQ ID NO: 239, where the polyN stretch of SEQ ID NO: 239 is the protospacer sequence complementary to the target DNA sequence.

In some embodiments, a gene knockout or inactivation comprises an insertion, a deletion, a mutation or a combination thereof, and made using an RNA-guided nuclease. Exemplary gRNA sequences and methods of using the same to knock out endogenous genes that encode immune cell proteins include those described in Ren et al., C/in Cancer Res. 2017. 23(9):2255-2266, the gRNAs, Cas9 DNAs, vectors, and gene knockout techniques of which are hereby expressly incorporated by reference in their entirety.

In some embodiments, a gene modification comprises an insertion of an exogenous nucleic acid sequence (e.g., heterologous promoter, transgene, and/or combinations thereof) into the genome of a cell, where an RNA-guided nuclease introduces a double-stranded break in the genome and the exogenous nucleic acid sequence is introduced into the genome by homology-directed repair.

In some embodiments, a genetic modification comprises insertion of an exogenous nucleic acid (e.g., donor template) into the TRAC locus of a cell genome, where the donor template comprises a 5′ homology arm and a 3′ homology arm, each having homology to nucleotide sequences within the TRAC locus, such that the exogenous nucleic acid is inserted into the TRAC locus following introduction of a double-stranded break within the TRAC locus. In some embodiments, the double-stranded break is introduced by an RNA-guided nuclease in the presence of a gRNA at a PAM sequence of NGG. In some embodiments, the double-stranded break is introduced by an RNA-guided nuclease in the presence of a gRNA at a PAM sequence of NNNNCC.

In some embodiments, the 5′ homology arm comprises a nucleotide sequence having at least 90% sequence identity to the nucleic acid sequence of SEQ ID NO: 85, the 3′ homology arm comprises a nucleotide sequence having at least 90% sequence identity to the nucleic acid sequence of SEQ ID NO: 93. In some embodiments, the 5′ homology arm comprises the nucleic acid sequence of SEQ ID NO: 85 and the 3′ homology arm comprises the nucleic acid sequence of SEQ ID NO: 93.

In some embodiments, the 5′ homology arm comprises a nucleotide sequence having at least 90% sequence identity to the nucleic acid sequence of SEQ ID NO: 96, the 3′ homology arm comprises a nucleotide sequence having at least 90% sequence identity to the nucleic acid sequence of SEQ ID NO: 105. In some embodiments, the 5′ homology arm comprises the nucleic acid sequence of SEQ ID NO: 96 and the 3′ homology arm comprises the nucleic acid sequence of SEQ ID NO: 105.

In some embodiments, the 5′ homology arm comprises a nucleotide sequence having at least 90% sequence identity to the nucleic acid sequence of SEQ ID NO: 108, the 3′ homology arm comprises a nucleotide sequence having at least 90% sequence identity to the nucleic acid sequence of SEQ ID NO: 116. In some embodiments, the 5′ homology arm comprises the nucleic acid sequence of SEQ ID NO: 108 and the 3′ homology arm comprises the nucleic acid sequence of SEQ ID NO: 116.

In some embodiments, the 5′ homology arm comprises a nucleotide sequence having at least 90% sequence identity to the nucleic acid sequence of SEQ ID NO: 119, the 3′ homology arm comprises a nucleotide sequence having at least 90% sequence identity to the nucleic acid sequence of SEQ ID NO: 127. In some embodiments, the 5′ homology arm comprises the nucleic acid sequence of SEQ ID NO: 119 and the 3′ homology arm comprises the nucleic acid sequence of SEQ ID NO: 127.

In some embodiments, the 5′ homology arm comprises a nucleotide sequence having at least 90% sequence identity to the nucleic acid sequence of SEQ ID NO: 130, the 3′ homology arm comprises a nucleotide sequence having at least 90% sequence identity to the nucleic acid sequence of SEQ ID NO: 138. In some embodiments, the 5′ homology arm comprises the nucleic acid sequence of SEQ ID NO: 130 and the 3′ homology arm comprises the nucleic acid sequence of SEQ ID NO: 138.

In some embodiments, a genetic modification comprises insertion of an exogenous nucleic acid (e.g., donor template) into the FOXP3 locus of a cell genome, where the donor template comprises a 5′ homology arm and a 3′ homology arm, each having homology to nucleotide sequences within the FOXP3 locus, such that the exogenous nucleic acid is inserted into the FOXP3 locus following introduction of a double-stranded break within the FOXP3 locus. In some embodiments, the double-stranded break is introduced by an RNA-guided nuclease in the presence of a gRNA at a PAM sequence of NGG. In some embodiments, the double-stranded break is introduced by an RNA-guided nuclease in the presence of a gRNA at a PAM sequence of NNNNCC.

In some embodiments, the 5′ homology arm comprises a nucleotide sequence having at least 90% sequence identity to the nucleic acid sequence of SEQ ID NO: 141, the 3′ homology arm comprises a nucleotide sequence having at least 90% sequence identity to the nucleic acid sequence of SEQ ID NO: 149. In some embodiments, the 5′ homology arm comprises the nucleic acid sequence of SEQ ID NO: 141 and the 3′ homology arm comprises the nucleic acid sequence of SEQ ID NO: 149.

In some embodiments, the 5′ homology arm comprises a nucleotide sequence having at least 90% sequence identity to the nucleic acid sequence of SEQ ID NO: 152, the 3′ homology arm comprises a nucleotide sequence having at least 90% sequence identity to the nucleic acid sequence of SEQ ID NO: 160. In some embodiments, the 5′ homology arm comprises the nucleic acid sequence of SEQ ID NO: 152 and the 3′ homology arm comprises the nucleic acid sequence of SEQ ID NO: 160.

In some embodiments, the 5′ homology arm comprises a nucleotide sequence having at least 90% sequence identity to the nucleic acid sequence of SEQ ID NO: 163, the 3′ homology arm comprises a nucleotide sequence having at least 90% sequence identity to the nucleic acid sequence of SEQ ID NO: 171. In some embodiments, the 5′ homology arm comprises the nucleic acid sequence of SEQ ID NO: 163 and the 3′ homology arm comprises the nucleic acid sequence of SEQ ID NO: 171.

In some embodiments, the 5′ homology arm comprises a nucleotide sequence having at least 90% sequence identity to the nucleic acid sequence of SEQ ID NO: 174, the 3′ homology arm comprises a nucleotide sequence having at least 90% sequence identity to the nucleic acid sequence of SEQ ID NO: 183. In some embodiments, the 5′ homology arm comprises the nucleic acid sequence of SEQ ID NO: 174 and the 3′ homology arm comprises the nucleic acid sequence of SEQ ID NO: 183.

In some embodiments, the 5′ homology arm comprises a nucleotide sequence having at least 90% sequence identity to the nucleic acid sequence of SEQ ID NO: 186, the 3′ homology arm comprises a nucleotide sequence having at least 90% sequence identity to the nucleic acid sequence of SEQ ID NO: 194. In some embodiments, the 5′ homology arm comprises the nucleic acid sequence of SEQ ID NO: 186 and the 3′ homology arm comprises the nucleic acid sequence of SEQ ID NO: 194.

In some embodiments, the 5′ homology arm comprises a nucleotide sequence having at least 90% sequence identity to the nucleic acid sequence of SEQ ID NO: 197, the 3′ homology arm comprises a nucleotide sequence having at least 90% sequence identity to the nucleic acid sequence of SEQ ID NO: 205. In some embodiments, the 5′ homology arm comprises the nucleic acid sequence of SEQ ID NO: 197 and the 3′ homology arm comprises the nucleic acid sequence of SEQ ID NO: 205.

In some embodiments, the 5′ homology arm comprises a nucleotide sequence having at least 90% sequence identity to the nucleic acid sequence of SEQ ID NO: 208, the 3′ homology arm comprises a nucleotide sequence having at least 90% sequence identity to the nucleic acid sequence of SEQ ID NO: 217. In some embodiments, the 5′ homology arm comprises the nucleic acid sequence of SEQ ID NO: 208 and the 3′ homology arm comprises the nucleic acid sequence of SEQ ID NO: 217.

### Cell Types

Embodiments of methods and systems for producing genetically modified cells (e.g., by in vitro or ex vivo gene editing) may use any cell type known in the art as a material for, e.g., introduction of nucleic acids, vectors, and/or compositions. It is to be understood that methods described herein that comprise manipulation of CD4+ cells, can be applied to other types of cells (e.g., CD8+ cells). In some embodiments, the methods described herein comprise editing an immune cell. Non-limiting examples of immune cells include B cells, T cells, and NK cells. In some embodiments, the methods provided herein comprise editing CD3+ cells, thereby producing edited CD3+ cells, including CD4+ and CD8+ Treg cells. In some embodiments, the methods comprise editing CD4+ T cells, thereby producing CD4+ Treg cells. In some embodiments, the methods comprise editing CD8+ T cells, thereby producing CD8+ Treg cells. In some embodiments, the methods comprise editing NK1.1+ T cells, thereby producing NK1.1+ Treg cells.

In some embodiments, the methods comprise editing a stem cell. In some embodiments, the methods comprise editing a pluripotent stem cell. In some embodiments, the methods comprise editing CD34+ hematopoietic stem cells (HSCs). In some embodiments, the methods comprise editing induced pluripotent stem cells (iPSCs). Edited stem cells may be matured in vitro to produce Treg cells. Edited stem cells may be matured into CD3+ Treg cells, CD4+ Treg cells, CD8+ Treg cells, NK1.1+ Treg cells, or a combination thereof.

In some embodiments, a method comprises editing a T cell. A T cell or T lymphocyte is an immune system cell that matures in the thymus and produces a T cell receptor (TCR), e.g., an antigen-specific heterodimeric cell surface receptor typically comprised of an α-βheterodimer or a γ-δ heterodimer. T cells of a given clonality typically express only a single TCR clonotype that recognizes a specific antigenic epitope presented by a syngeneic antigen-presenting cell in the context of a major histocompatibility complex-encoded determinant. T cells can be naïve (“TN”; not exposed to antigen; increased expression of CD62L, CCR7, CD28, CD3, CD127, and CD45RA, and decreased or no expression of CD45RO as compared to TcM (described herein)), memory T cells (T_{M}) (antigen experienced and long-lived), including stem cell memory T cells, and effector cells (antigen-experienced, cytotoxic). TM can be further divided into subsets of central memory T cells (TcM, expresses CD62L, CCR7, CD28, CD95, CD45RO, and CD127) and effector memory T cells (TEM, express CD45RO, decreased expression of CD62L, CCR7, CD28, and CD45RA). Effector T cells (Teff) refers to antigen-experienced CD8+ cytotoxic T lymphocytes that express CD45RA, have decreased expression of CD62L, CCR7, and CD28 as compared to TcM, and are positive for granzyme and perform. Helper T cells (TH) are CD4+ cells that influence the activity of other immune cells by releasing cytokines. CD4+ T cells can activate and suppress an adaptive immune response, and which of those two functions is induced will depend on the presence of other cells and signals. T cells can be collected using known techniques, and the various subpopulations or combinations thereof can be enriched or depleted by known techniques, for example, using antibodies that specifically recognize one or more T cell surface phenotypic markers, by affinity binding to antibodies, flow cytometry, fluorescence activated cell sorting (FACS), or immunomagnetic bead selection. Other exemplary T cells include regulatory T cells (Treg, also known as suppressor T cells), such as CD4+CD25+(FoxP3+) regulatory T cells and Treg17 cells, as well as Tr1, Th3, CD8+CD28−, or Qa-1 restricted T cells. In some embodiments, the cell is a CD3+, CD4+, and/or CD8+ T cell. In some embodiments, the cell is a CD3+ T cell. In some embodiments, the cell is a CD4⁺CD8⁻ T cell. In some embodiments, the cell is a CD4⁻CD8⁺ T cell. In some embodiments, the cell is a regulatory T cell (Treg). Non-limiting examples of Treg cells are Tr1, Th3, CD8+CD28−, and Qa-1 restricted T cells. In some embodiments, the Treg cell is a FoxP3+ Treg cell. In some embodiments, the Treg cell expresses CTLA-4, LAG-3, CD25, CD39, CD27, CD70, CD357 (GITR), neuropilin-1, galectin-1, and/or IL-2Rα on its surface.

In some embodiments, the cell is a human cell. In some embodiments, a cell as described herein is isolated from a biological sample. A biological sample may be a sample from a subject (e.g., a human subject) or a composition produced in a lab (e.g., a culture of cells). A biological sample obtained from a subject make be a liquid sample (e.g., blood or a fraction thereof, a bronchial lavage, cerebrospinal fluid, or urine), or a solid sample (e.g., a piece of tissue) In some embodiments, the cell is obtained from peripheral blood. In some embodiments, the cell is obtained from umbilical cord blood. In some embodiments, the cell is obtained by sorting cells of peripheral blood to obtain a desired cell population (e.g., CD3+ cells), and one or more cells of the sorted population are modified by a method described herein. Also contemplated herein are cells produced by a method described herein.

Embodiments of genetically modified cells described herein are Treg cells. Non-limiting examples of Treg cells are Tr1, Th3, CD8+CD28−, and Qa-1 restricted T cells. In some embodiments, the cell is an NK-T cell (e.g., a FoxP3+ NK-T cell). In some embodiments, the cell is a CD4+ T cell (e.g., a FoxP3+CD4+ T cell) or a CD8+ T cell (e.g., a FoxP3+CD8+ T cell). In some embodiments, the cell is a CD25− T cell. In some embodiments, the Treg cell is a FoxP3+ Treg cell. In some embodiments, the Treg cell expresses CTLA-4, LAG-3, CD25, CD39, CD27, CD70, CD357 (GITR), neuropilin-1, galectin-1, and/or IL-2Rα on its surface. In some embodiments, the Treg cell is CTLA-4+. In some embodiments, the Treg cell is LAG-3+. In some embodiments, the Treg cell is CD25+. In some embodiments, the Treg cell is CD39+. In some embodiments, the Treg cell is CD27+. In some embodiments, the Treg cell is CD70+. In some embodiments, the Treg cell is CD357+. In some embodiments, the Treg cell is IL-2Rα+. In some embodiments, the Treg cell expresses IL-2Rβ and IL-2Rγ on its surface. In some embodiments, the Treg cell expresses neuropilin-1 on it surface. In some embodiments, the Treg cell expresses galectin-1 on its surface.

### Polynucleotides, Polypeptides, and Sequence Identity

Aspects of the disclosure relate to nucleic acids for insertion into cell genomes (e.g., in methods or systems), and genetically modified cells comprising inserted nucleic acids. As will be understood by those skilled in the art, nucleic acids may include genomic sequences, extra-genomic and plasmid-encoded sequences and smaller engineered gene segments that express, or may be adapted to express, proteins, polypeptides, peptides and the like. Such segments may be naturally isolated or modified synthetically by the skilled person.

As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence encoding a variant or derivative of such a sequence.

In some embodiments, polynucleotide variants may have substantial identity to a reference polynucleotide sequence encoding an immunomodulatory polypeptide described herein. For example, a polynucleotide may be a polynucleotide comprising at least 70% sequence identity, preferably at least 75%, 80%, 85%, 90%, 95%, 96%, 97%, 98%, or 99% or higher, sequence identity or a sequence identity that is within a range defined by any two of the aforementioned percentages as compared to a reference polynucleotide sequence such as a sequence encoding an antibody described herein, using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the binding affinity of a polypeptide variant of a given polypeptide which is capable of a specific binding interaction with another molecule and is encoded by the variant polynucleotide is not substantially diminished relative to a polypeptide encoded by a polynucleotide sequence specifically set forth herein.

Some embodiments of nucleic acid sequences described herein (e.g., sequences on nucleic acids, vectors, or in cells) are codon-optimized for expression in a cell. The terms “codon-optimized” and “codon optimization,” with respect to a gene or coding sequence present in or introduced into a host cell, refer to alteration of codons in the gene or coding sequence to reflect the typical codon usage of the host cell, without altering the amino acid sequence encoded by the gene or coding sequence. Such optimization includes replacing at least one, or more than one, or a significant number, of codons with one or more codons that are more frequently used in the genes of that organism. Codon usage tables are readily available, for example, at the “Codon Usage Database” available at www.kazusa.or.jp. By utilizing the knowledge on codon usage or codon preference in each organism, one of ordinary skill in the art can apply the frequencies to any polypeptide with a given amino acid sequence, to produce a codon-optimized coding sequence which encodes the same polypeptide having the same amino acid sequence, but uses codons optimal for a given species (e.g., a human). Codon-optimized coding regions can be designed by various methods known to those skilled in the art.

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of or about of 10,000, 5000, 3000, 2,000, 1,000, 500, 200, 100, or 50 base pairs in length, and the like, (including all intermediate lengths) are contemplated to be useful.

When comparing polynucleotide or nucleic acid sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least or at least about 20 contiguous positions, usually 30 to 75, or 40 to 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., Unified Approach to Alignment and Phylogenes, pp. 626-645 (1990); Methods in Enzymology vol. 183, Academic Press, Inc., San Diego, CA; Higgins, D. G. and Sharp, P. M., CABIOS 5:151-153 (1989); Myers, E. W. and Muller W., CABIOS 4:11-17 (1988); Robinson, E. D., Comb. Theor 11:105 (1971); Santou, N. Nes, M., Mol. Biol. Evol. 4:406-425 (1987); Sneath, P. H. A. and Sokal, R. R., Numerical Taxonomy—the Principles and Practice of Numerical Taxonomy, Freeman Press, San Francisco, CA (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One preferred example of algorithms that are suitable for determining percent sequence identity and sequence similarity are the BLAST and BLAST 2.0 algorithms, which are described in Altschul et al., Nucl Acids Res. 1977. 25:3389-3402, and Altschul et al., J Mol Biol. 1990. 215:403-410, respectively. BLAST and BLAST 2.0 can be used, for example with the parameters described herein, to determine percent sequence identity among two or more the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff, Proc Natl Acad Sci USA. 1989. 89:10915) alignments, (B) of 50, expectation (E) of 10, M=5, N=−4 and a comparison of both strands.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

### Pharmaceutical Compositions

Some aspects of the disclosure relate to a pharmaceutical composition comprising a cell, vector, or nucleic acid described herein, and a pharmaceutically acceptable excipient or carrier. Such pharmaceutical compositions are formulated, for example, for systemic administration, or administration to target tissues. “Acceptable” means that the excipient (carrier) must be compatible with the active ingredient of the composition (and preferably, capable of stabilizing the active ingredient) and not deleterious to the subject to be treated. Pharmaceutically acceptable excipients, carriers, buffers, stabilizers, isotonicizing agents, preservatives or antioxidants, or other materials well known to those skilled in the art. Such materials should be non-toxic and should not interfere with the efficacy of the active ingredient. The precise nature of the carrier or other material may depend on the route of administration, e.g., parenteral, intramuscular, intradermal, sublingual, buccal, ocular, intranasal, subcutaneous, intrathecal, intratumoral, oral, vaginal, or rectal. See, e.g., Remington: The Science and Practice of Pharmacy 20th Ed. (2000) Lippincott Williams and Wilkins, Ed. K. E. Hoover. The pharmaceutical compositions to be used for in vivo administration must be sterile, with the exception of any cells, viruses, and/or viral vectors being used to achieve a biological effect (e.g., immunosuppression). This is readily accomplished by, for example, filtration through sterile filtration membranes. The pharmaceutical compositions described herein may be placed into a container having a sterile access port, for example, an intravenous solution bag or vial having a stopper pierceable by a hypodermic injection needle.

In some embodiments, the pharmaceutical compositions described herein can be formulated for intramuscular injection, intravenous injection, intradermal injection, or subcutaneous injection.

The pharmaceutical compositions described herein to be used in contemplated methods can comprise pharmaceutically acceptable carriers, buffer agents, excipients, salts, or stabilizers in the form of lyophilized formulations or aqueous solutions. See, e.g., Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

Pharmaceutical compositions described herein may be useful for treating a subject that has or is at risk of developing type 1 diabetes (T1D). A subject having or at risk of developing type 1 diabetes or disease may be identified by ascertaining the presence and/or absence of one or more risk factors, diagnostic indicators, or prognostic indications. The determination may be made based on clinical, cellular, or serologic findings, including flow cytometry, serology, and/or DNA analyses known in the art.

The pharmaceutical compositions described herein can include a therapeutically effective amount of any cell, vector, and/or nucleic acid described herein. For example, in some embodiments, the pharmaceutical composition includes a cell, vector, or nucleic acid at any of the doses described herein.

A “therapeutically effective amount” refers to an amount effective, at dosages and for periods of time necessary, to achieve a desired therapeutic result. The therapeutically effective amount may vary according to factors such as the age, sex, and weight of the individual, and the ability of the cell, nucleic acid, or vector to effect a desired response in the subject.

Pharmaceutical compositions can be prepared in accordance with known techniques. See, e.g., Remington, The Science And Practice of Pharmacy (21st ed., Philadelphia, Lippincott, Williams & Wilkins, 2005). For example, cells, vectors, or nucleic acids described herein may be admixed with a pharmaceutically acceptable excipient, and the resulting composition is administered to a subject. The carrier must be acceptable in the sense of being compatible with any other ingredients in the formulation and must not be deleterious to the subject. The carrier can be a solid or a liquid, or both, and can be formulated with the compound as a unit-dose formulation.

In some embodiments, a pharmaceutical composition comprises cells at a dose of about 10⁴ to about 10¹⁰ cells/kg. In some embodiments, the pharmaceutical composition comprises cells at a dose of about: 10⁴ to 10⁵, 10⁵ to 10⁶, 10⁶ to 10⁷, 10⁷ to 10⁸, 10⁸ to 10⁹, or 10⁹ to 10¹⁰ cells/kg. In some embodiments, a pharmaceutical composition comprises cells at a dose of about 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, 5.0×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶, 6.0×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7.0×10⁶, 7.1×10⁶, 7.2×10⁶, 7.3×10⁶, 7.4×10⁶, 7.5×10⁶, 7.6×10⁶, 7.7×10⁶, 7.8×10⁶, 7.9×10⁶, 8.0×10⁶, 8.1×10⁶, 8.2×10⁶, 8.3×10⁶, 8.4×10⁶, 8.5×10⁶, 8.6×10⁶, 8.7×10⁶, 8.8×10⁶, 8.9×10⁶, 9.0×10⁶, 9.1×10⁶, 9.2×10⁶, 9.3×10⁶, 9.4×10⁶, 9.5×10⁶, 9.6×10⁶, 9.7×10⁶, 9.8×10⁶, 9.9×10⁶, 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 2.1×10⁷, 2.2×10⁷, 2.3×10⁷, 2.4×10⁷, 2.5×10⁷, 2.6×10⁷, 2.7×10⁷, 2.8×10⁷, 2.9×10⁷, 3.0×10⁷, 3.1×10⁷, 3.2×10⁷, 3.3×10⁷, 3.4×10⁷, 3.5×10⁷, 3.6×10⁷, 3.7×10⁷, 3.8×10⁷, 3.9×10⁷, 4.0×10⁷, 4.1×10⁷, 4.2×10⁷, 4.3×10⁷, 4.4×10⁷, 4.5×10⁷, 4.6×10⁷, 4.7×10⁷, 4.8×10⁷, 4.9×10⁷, 5.0×10⁷, 5.1×10⁷, 5.2×10⁷, 5.3×10⁷, 5.4×10⁷, 5.5×10⁷, 5.6×10⁷, 5.7×10⁷, 5.8×10⁷, 5.9×10⁷, 6.0×10⁷, 6.1×10⁷, 6.2×10⁷, 6.3×10⁷, 6.4×10⁷, 6.5×10⁷, 6.6×10⁷, 6.7×10⁷, 6.8×10⁷, 6.9×10⁷, 7.0×10⁷, 7.1×10⁷, 7.2×10⁷, 7.3×10⁷, 7.4×10⁷, 7.5×10⁷, 7.6×10⁷, 7.7×10⁷, 7.8×10⁷, 7.9×10⁷, 8.0×10⁷, 8.1×10⁷, 8.2×10⁷, 8.3×10⁷, 8.4×10⁷, 8.5×10⁷, 8.6×10⁷, 8.7×10⁷, 8.8×10⁷, 8.9×10⁷, 9.0×10⁷, 9.1×10⁷, 9.2×10⁷, 9.3×10⁷, 9.4×10⁷, 9.5×10⁷, 9.6×10⁷, 9.7×10⁷, 9.8×10⁷, 9.9×10⁷, or 1.0×10⁸ cells/kg.

In some embodiments, pharmaceutical compositions described herein can further comprise one or more additional agents useful in the treatment of type 1 diabetes in a subject.

### METHODS OF USE

Some aspects of the disclosure relate to methods of administering a genetically modified cell described herein to a subject. In some embodiments, a method comprises administering to a subject any one of the genetically modified cells described herein. In some embodiments, a method comprises administering to the subject a cell that had previously been obtained from that subject before being administered (i.e., the cell is an autologous cell). In some embodiments, a method comprises (i) isolation of cells from a subject; (ii) processing the cells by any method (e.g., gene editing and/or introducing a vector) described herein; and (iii) administering the processed cells to the same subject. In some embodiments, a method comprises administering to the subject a cell that had previously been obtained from a different subject than the one to whom the cell is administered (i.e., the cell is an allogeneic cell). In some embodiments, a method comprises (i) isolation of cells from a first subject; (ii) processing the cells by any method (e.g., gene editing or introducing a vector) described herein; and (iii) administering the processed cells to a second subject.

Some embodiments of the methods, cells, systems, and compositions described herein include any of the cells, vectors, nucleic acids, or lipid nanoparticles described herein, for use as a medicament. In some embodiments, the cell, vector, nucleic acid, or lipid nanoparticle is for use in a method of preventing, treating, inhibiting, or ameliorating type 1 diabetes in a subject.

In some embodiments, a cell is described herein for use in a method of preventing, treating, inhibiting, or ameliorating type 1 diabetes in a subject. In some embodiments, the cell is autologous to the subject (i.e., derived from the subject). In other embodiments, the cell is allogeneic to the subject (i.e., derived from a different subject).

In some embodiments, the cell expresses an antigen-specific receptor (e.g., T cell receptor) that is specific to an antigen associated with type 1 diabetes. In some embodiments, the TCR is a T1D2 TCR that binds a peptide of IGRP(305-324) in an HLA-DRB1*0401-restricted manner. In some embodiments, the TCR is a T1D4 TCR that binds a peptide of IGRP(241-260) in an HLA-DRB1*0401-restricted manner. In some embodiments, the TCR is a T1D5-1 TCR that binds a peptide of IGRP(305-324) in an HLA-DRB1*0401-restricted manner.

### Administration

In some embodiments, a genetically modified cell may be administered between 1 and 14 days over a 30-day period. In some embodiments, doses may be provided 1, 2, 3, 4, 5, 6, 7, 8, 9, 10, 11, 12, 13, or 14 days over a 60-day period. Alternate protocols may be appropriate for individual subjects. A suitable dose is an amount of a compound that, when administered as described above, is capable of detectably altering or ameliorating symptoms, or decreases at least one indicator of type 1 diabetes in a statistically significant manner by at least 10-50% relative to the basal (e.g., untreated) level, which can be monitored by measuring specific levels of blood components, e.g., detectable levels of circulating immunocytes and/or other inflammatory cells and/or soluble inflammatory mediators including proinflammatory cytokines.

In some embodiments, rapamycin or a rapalog is administered to the subject before the administration of cells, in conjunction with cells, and/or following the administration of cells. Administration of rapamycin that is capable of inducing dimerization of the CISC components on the surface of a cell results in continued IL-2 signal transduction in vivo, promoting survival and proliferation of the CISC-expressing cell without the undesired effects that would be caused by IL-2 administration, such as activation of other T cells. Similarly, in vivo metabolism of a rapalog to produce rapamycin or a molecule with similar structure capable of inducing heterodimerization of the CISC components at the surface of the cell results in in vivo IL-2 signal transduction in the engineered cells, promoting survival and proliferation. In some embodiments, the compound produced by in vivo metabolism of the rapalog is rapamycin. In some embodiments, the rapalog that is administered is everolimus, CCI-779, C20-methallylrapamycin, C16-(S)-3-methylindolerapamycin, C16-iRap, C16-(S)-7-methylindolerapamycin, AP21967, C16-(S)Butylsulfonamidorapamycin, AP23050, sodium mycophenolic acid, benidipine hydrochloride, AP1903, and AP23573, or a metabolite or derivative thereof. In some embodiments, the rapamycin or rapalog is administered at a dose of 0.001 mg/kg to 10 mg/kg body mass of the subject, or a dose between 0.001 mg/kg and 10 mg/kg. In some embodiments, the rapamycin or rapalog is administered at a dose of 0.001 mg/kg to 0.01 mg/kg, 0.01 mg/kg to 0.1 mg/kg, 0.1 mg/kg to 1 mg/kg, or 1 mg/kg to 10 mg/kg. In some embodiments, the rapamycin or rapalog is administered in a separate composition from the cells. In some embodiments, the rapamycin or rapalog is administered in multiple doses. In some embodiments, the rapamycin or rapalog is administered for 1, 2, 3, 4, 5, 6, 7, 8, 9, 10, 11, 12, 13, or 14 or more days. In some embodiments, the rapamycin or rapalog is administered for 1, 2, 3, 4, 5, 6, 7, 8, 9, 10, 11, 12, 13, 14, 15, 16, 17, 18, 19, or 20 or more weeks. In some embodiments, the subject is a human. In some embodiments, the administration of the rapamycin or rapalog results in prolonged survival of the administered cells, relative to a subject that is not administered rapamycin or a rapalog. In some embodiments, the administration of the rapamycin or rapalog increases the frequency of cells circulating in the peripheral blood of a subject, relative to a subject that is not administered rapamycin or a rapalog.

In general, an appropriate dosage and treatment regimen provides the cells in an amount sufficient to provide therapeutic and/or prophylactic benefit. Such a response can be monitored by establishing an improved clinical outcome (e.g., more frequent remissions, complete or partial, or longer disease-free survival) in treated subjects as compared to non-treated subjects. Decreases (e.g., reductions having statistical significance when compared to a relevant control) in preexisting immune responses to an antigen associated with type 1 diabetes as provided herein generally correlate with an improved clinical outcome. Such immune responses may generally be evaluated using standard leukocyte and/or lymphocyte cell surface marker or cytokine expression, proliferation, cytotoxicity or released cytokine assays, which are routine in the art and may be performed using samples obtained from a subject before and after therapy.

In some embodiments, engineered cells described herein may be administered to a subject after identifying the presence of one or more signs or risk factors of T1D. The appearance of anti-islet autoantibodies in peripheral blood is the most reliable marker to signal the presence of an autoimmune process against the pancreas. Specifically, these autoantibodies reflect targeting of beta cells by the immune system. Non-limiting examples of autoantibodies that may be measured to determine whether a subject has, is developing, or is at risk of developing T1D include antibodies that bind islet cells, insulin, glutamic acid decarboxylase, islet tyrosine phosphatase 2, and/or zinc transporter 8. In children with multiple T1D-associated autoantibodies, approximately 70% were found to progress to clinical T1D over a 10-year follow-up period, compared with less than 1% of children with no anti-islet autoantibodies; an increased risk for development of clinical T1D was seen in those who had multiple autoantibodies present before 3 years of age. See Ziegler et al., JAMA. 2013. 309(23):2473-2479. In some embodiments a subject is administered an engineered cell after detection of one or more antibodies specific to an islet cell antigen, insulin, glutamic acid decarboxylase, islet tyrosine phosphatase 2, and/or zinc transporter 8.

In some embodiments, a subject administered engineered cells described herein has not been diagnosed with T1D more than 6 months prior to administration of the cells. Administration of EngTregs as described herein shortly after the onset of T1D, or before T1D onset but following detection of one or more risk factors indicative of T1D development (e.g., autoantibodies), is useful, in some embodiments, for preserving pancreatic function by mitigating autoimmune responses towards the pancreas before a substantial portion or majority of islet cells are damaged or depleted. In some embodiments, the subject has not been diagnosed with T1D more than 5 months, 4 months, 3 months, 2 months, or 1 month prior to administration of the cells. In some embodiments, a subject is administered engineered cells within 6 months of receiving a diagnosis of T1D. In some embodiments, a subject is administered engineered cells no more than 5, 4, 3, 2, or 1 month after being diagnosed with T1D. A subject may not have been diagnosed with T1D at all, but administered the cells after detection of autoantibodies specific to 1, 2, 3, 4, or 5 antigens selected from islet cell antigen, insulin, glutamic acid decarboxylase, islet tyrosine phosphatase 2, and/or zinc transporter 8. In some embodiments, the subject is administered engineered cells without being diagnosed with T1D, but after detection of autoantibodies in serum that are specific to islet cell antigen, insulin, glutamic acid decarboxylase, islet tyrosine phosphatase 2, and/or zinc transporter 8. In some embodiments, the subject is administered engineered cells within 6 months after the first detection of autoantibodies specific to islet cell antigen, insulin, glutamic acid decarboxylase, islet tyrosine phosphatase 2, and/or zinc transporter 8. In some embodiments, the subject is administered engineered cells within 6, 5, 4, 3, 2, or 1 months after the first detection of autoantibodies specific to islet cell antigen in serum. In some embodiments, the subject is administered engineered cells within 6, 5, 4, 3, 2, or 1 months after the first detection of autoantibodies specific to insulin in serum. In some embodiments, the subject is administered engineered cells within 6, 5, 4, 3, 2, or 1 months after the first detection of autoantibodies specific to glutamic acid decarboxylase in serum. In some embodiments, the subject is administered engineered cells within 6, 5, 4, 3, 2, or 1 months after the first detection of autoantibodies specific to islet tyrosine phosphatase 2 in serum. In some embodiments, the subject is administered engineered cells within 6, 5, 4, 3, 2, or 1 months after the first detection of autoantibodies specific to zinc transporter 8 in serum.

In some embodiments, EngTregs may be administered to a subject in diabetic remission. One form of remission generally occurs shortly after the initiation of exogenous insulin therapy, during which time the need for exogenous insulin may decrease. The subject to which engineered cells are administered may be a subject with partial clinical remission, defined as having an insulin dose-adjusted hemoglobin A1c (HbA1c) (IDAA1c) of 9 or less. Methods of measuring HbA1c, and calculating insulin-adjusted HbA1c are known in the art. See, e.g., Mortensen et al., 2009. In some embodiments, a subject has an insulin dose-adjusted HbA1c of 9 or less, calculated using the formula: IDAA1c=HbA1c (%)+(4)(insulin dose [IU/kg/24 h]). In some embodiments, EngTregs are administered within 6, 5, 4, 3, 2, or 1 months after a subject enters diabetic remission. In some embodiments, EngTregs are administered after a subject has been diagnosed with T1D, and the subject's insulin dose-adjusted HbA1c has decreased to 9.0 or lower. In some embodiments, the subject's insulin dose-adjusted HbA1c has decreased below 9.0, and an insulin dose-adjusted HbA1c above 9.0 has not been detected since the decrease below 9.0. In some embodiments, the subject's insulin dose-adjusted HbA1c has decreased to 9.0 or below after T1D diagnosis, and their insulin dose-adjusted HbA1c at the time of engineered cell administration is 9.0 or below. In some embodiments, the subject's insulin dose-adjusted HbA1c is 9 or lower, 8.9 or lower, 8.8 or lower, 8.7 or lower, 8.6 or lower, 8.5 or lower, 8.4 or lower, 8.3 or lower, 8.2 or lower, 8.1 or lower, 8.0 or lower, 7.9 or lower, 7.8 or lower, 7.7 or lower, 7.6 or lower, 7.5 or lower, 7.4 or lower, 7.3 or lower, 7.2 or lower, 7.1 or lower, 7.0 or lower, 6.9 or lower, 6.8 or lower, 6.7 or lower, 6.6 or lower, 6.5 or lower, 6.4 or lower, 6.3 or lower, 6.2 or lower, 6.1 or lower, 6.0 or lower, 5.9 or lower, 5.8 or lower, 5.7 or lower, 5.6 or lower, or 5.5 or lower.

Engineered cells described herein may also be administered to a subject with HbA1c levels that indicate prediabetes. In some embodiments, a subject is considered prediabetic if they have an unadjusted HbA1c of 5.7 to 6.4. In some embodiments, a subject's HbA1c, without adjusting for insulin dose, is 5.7 to 6.4. In some embodiments, a subject's HbA1c, without adjusting for insulin dose, is 5.7, 5.8, 5.9, 6.0, 6.1, 6.2, 6.3, or 6.4.

Engineered cells may be administered to a subject with HbA1c levels that indicate diabetes. In some embodiments, a subject is considered diabetic if they have an unadjusted HbA1c of 6.5 or higher (e.g., 6.5-10). In some embodiments, a subject's non-adjusted HbA1c is 6.5 to 10.0. In some embodiments, a subject's non-adjusted HbA1c is 6.5 to 10.0. In some embodiments, a subject's non-adjusted HbA1c is 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or 10.

In some embodiments, an appropriate dosage and treatment regimen is determined based on the age, expected pancreatic volume, and/or actual pancreatic volume of the subject. Administering a number of cells based on a subject's age, expected pancreatic volume, and/or actual pancreatic volume allows for normalization of the number of engineered cells that are expected to engraft in a subject's pancreas. For example, a younger subject with a developing pancreas is expected to have a smaller pancreatic volume than an older child or adult, and so a smaller dose is sufficient to achieve engraftment of a given number of cells relative to pancreas volume.

In some embodiments, a subject is 3 to 6 years of age, with mean pancreas volume in a healthy subject in this age range being about 20 mL. In some embodiments, a subject aged 3 to 6 years is administered a dose of 3×10⁸ cells. In some embodiments, a subject aged 3 to 6 years is administered a dose of 1×10⁸ to 6×10⁸ cells. In some embodiments, a subject aged 3 to 6 years is administered a dose of 1×10⁸ to 2×10⁸, 2×10⁸ to 3×10⁸, 3×10⁸ to 4×10⁸, 4×10⁸ to 5×10⁸, or 5×10⁸ to 6×10⁸ cells.

In some embodiments, a subject is 6 to 12 years of age, with mean pancreas volume in a healthy subject in this age range being about 35 mL. In some embodiments, a subject aged 6 to 12 years is administered a dose of 5×10⁸ cells. In some embodiments, a subject aged 6 to 12 years is administered a dose of 2×10⁸ to 1×10⁹ cells. In some embodiments, a subject aged 6 to 12 years is administered a dose of 2×10⁸ to 3×10⁸, 3×10⁸ to 4×10⁸, 4×10⁸ to 5×10⁸, 5×10⁸ to 6×10⁸, 6×10⁸ to 7×10⁸ cells, 7×10⁸ to 8×10⁸ cells, 8×10⁸ to 9×10⁸ cells, or 9×10⁸ to 1×10⁹ cells.

In some embodiments, a subject is 12 to 18 years of age, with mean pancreas volume in a healthy subject in this age range being about 60 mL. In some embodiments, a subject aged 12 to 18 years is administered a dose of 1×10⁹ cells. In some embodiments, a subject aged 12 to 18 years is administered a dose of 5×10⁸ to 2×10⁹ cells. In some embodiments, a subject aged 12 to 18 years is administered a dose of 5×10⁸ to 6×10⁸, 6×10⁸ to 7×10⁸ cells, 7×10⁸ to 8×10⁸ cells, 8×10⁸ to 9×10⁸ cells, 9×10⁸ to 1×10⁹ cells, 1×10⁹ to 1.1×10⁹, 1.1×10⁹ to 1.2×10⁹, 1.2×10⁹ to 1.3×10⁹, 1.3×10⁹ to 1.4×10⁹, 1.4×10⁹ to 1.5×10⁹, 1.5×10⁹ to 1.6×10⁹, 1.6×10⁹ to 1.7×10⁹, 1.7×10⁹ to 1.8×10⁹, 1.8×10⁹ to 1.9×10⁹, or 1.9×10⁹ to 2.0×10⁹ cells.

In some embodiments, a subject is 18 to 46 years of age, with mean pancreas volume in a healthy subject in this age range being about 70 mL. In some embodiments, a subject aged 18 to 46 years is administered a dose of 1×10⁹ cells. In some embodiments, a subject is at least 46 years old, and is administered a dose of 10⁹ cells. In some embodiments, a subject aged 12 to 18 years is administered a dose of 5×10⁸ to 2×10⁹ cells. In some embodiments, a subject aged 12 to 18 years is administered a dose of 5×10⁸ to 6×10⁸, 6×10⁸ to 7×10⁸ cells, 7×10⁸ to 8×10⁸ cells, 8×10⁸ to 9×10⁸ cells, 9×10⁸ to 1×10⁹ cells, 1×10⁹ to 1.1×10⁹, 1.1×10⁹ to 1.2×10⁹, 1.2×10⁹ to 1.3×10⁹, 1.3×10⁹ to 1.4×10⁹, 1.4×10⁹ to 1.5×10⁹, 1.5×10⁹ to 1.6×10⁹, 1.6×10⁹ to 1.7×10⁹, 1.7×10⁹ to 1.8×10⁹, 1.8×10⁹ to 1.9×10⁹, or 1.9×10⁹ to 2.0×10⁹ cells.

In some embodiments, the subject is 3 to 6 years old, and is administered a dose between 80% and 120% of 3×10⁸ cells (2.4×10⁸ to 3.6×10⁸ cells). In some embodiments, the subject is 6 to 12 years old, and is administered a dose between 80% and 120% of 5×10⁸ cells (4×10⁸ to 6×10⁸ cells). In some embodiments, the subject is 12 to 18 years old, and is administered a dose between 80% and 120% of 1×10⁹ cells (8×10⁸ to 1.2×10⁹ cells). In some embodiments, the subject is 18 to 46 years old, and is administered a dose between 80% and 120% of 1×10⁹ cells (8×10⁸ to 1.2×10⁹ cells). In some embodiments, the subject is at least 46 years old, and is administered a dose between 80% and 120% of 1×10⁹ cells (8×10⁸ to 1.2×10⁹ cells).

In some embodiments, the actual pancreatic volume of a subject is measured to calculate an administered cell dose. In some embodiments, the actual pancreatic volume of a subject is estimated using one of any method known in the art, such as an MRI or CT scan and further image analysis, e.g., as described in Qiu et al., Pediatr Radiol. 2022. doi: 10.1007/s00247-022-05405-8. In some embodiments, an administered dose of cells is adjusted proportionally to the ratio of a subject's actual pancreas volume to the mean pancreas volume for a healthy subject of similar age. For example, a subject aged 18 to 46 years and having a pancreas volume of 49 mL, where mean pancreas volume in similarly aged healthy subjects is 70 mL, would have an actual pancreas volume of 70% (49/70) relative to expected pancreas volume, and so would receive a dose of about 70% as many cells as would be used based on an expected volume of 70 mL (7×10⁸ cells, being 70% of 10⁹ cells based on expected volume).

In some embodiments, the subject is a human. In some embodiments, the subject is an animal. In some embodiments, the animal is a research animal. In some embodiments, the animal is a domesticated animal. In some embodiments, the animal is a rodent. In some embodiments, the rodent is a mouse, rat, guinea pig, chinchilla, or hamster. In some embodiments, the animal is a dog, cat, rabbit, guinea pig, hamster, or ferret. In some embodiments, the animal is a bovine, swine, llama, alpaca, sheep, or goat.

Certain aspects disclosed in the following publications are useful with embodiments of the methods, compositions, and systems provided herein: U.S. 2019/0247443; U.S. 2020/0123224; U.S. 2021/0340573; U.S. 2021/0253652; U.S. 2021/0054376; WO 2020/264039; and WO 2022/140354 which are each incorporated by reference in its entirety.

### EXAMPLES

### Example 1: Generation and Characterization of Immunosuppressive Capacity of Human T1D2 and T1D5-1 Expressing Dual-HDR Edited EngTregs for Use in T1D Therapy

Engineered Treg cells (EngTregs) products were generated for use in human subjects for prevention and/or treatment of Type 1 Diabetes (T1D) by dual-HDR-based editing. Two nucleic acids were inserted into the cell genome at separate loci.

A first inserted nucleic acid, inserted into the TRAC locus, included an MND promoter operably linked to a sequence encoding (i) a first transmembrane protein for rapamycin-inducible IL-2 signal transduction, having an FKBP extracellular domain linked to a transmembrane and intracellular domain of IL-2Rγ; (ii) a human TCRβ chain of T1D2 or T1D5-1, each of which is specific to a peptide of the islet antigen IGRP; and (iii) a portion of the TCRα chain of the T1D2 or T1D5-1 TCR, respectively (i.e., each nucleic acid inserted into the TRAC locus encoded the TCRβ chain of T1D2 and a portion of the TCRα chain of T1D2, or the TCRβ chain of T1D5-1 and a portion of the TCRα chain of T1D5-1). The nucleic acid was inserted into the TRAC locus such that the inserted sequence encoding a TCRα chain portion (including the variable domain determining antigen specificity) was in-frame with the endogenous sequence encoding the remaining portion of the TCRα chain (including the constant domain), such that a full-length TCRα chain was expressed from the TRAC locus under control of the inserted MND promoter, and expression of the endogenous TCRα chain (having different specificity) was disrupted.

The second inserted nucleic acid, inserted into the FOXP3 locus downstream from the Treg-specific demethylated region (TSDR), contained an MND promoter operably linked to a sequence encoding (i) a second transmembrane protein for rapamycin-inducible IL-2 signal transduction, having an FRB extracellular domain linked to a transmembrane and intracellular domain of IL-2Rβ; (ii) a cytosolic FRB domain to adsorb intracellular rapamycin and limit mTOR inhibition; and (iii) the endogenous FOXP3 coding sequence beginning with exon 2, which contains the endogenous start codon.

The dual-edited cells produced by insertion of both nucleic acids stably expressed, under control of the MND promoter: (i) both components of a rapamycin-inducible signaling complex, which heterodimerize in the presence of rapamycin to provide IL-2 signal transduction, thereby inducing cell proliferation; (ii) an IGRP-specific human TCR; (iii) FoxP3; and (iv) a cytosolic FRB domain for mitigating mTOR inhibition in the presence of rapamycin.

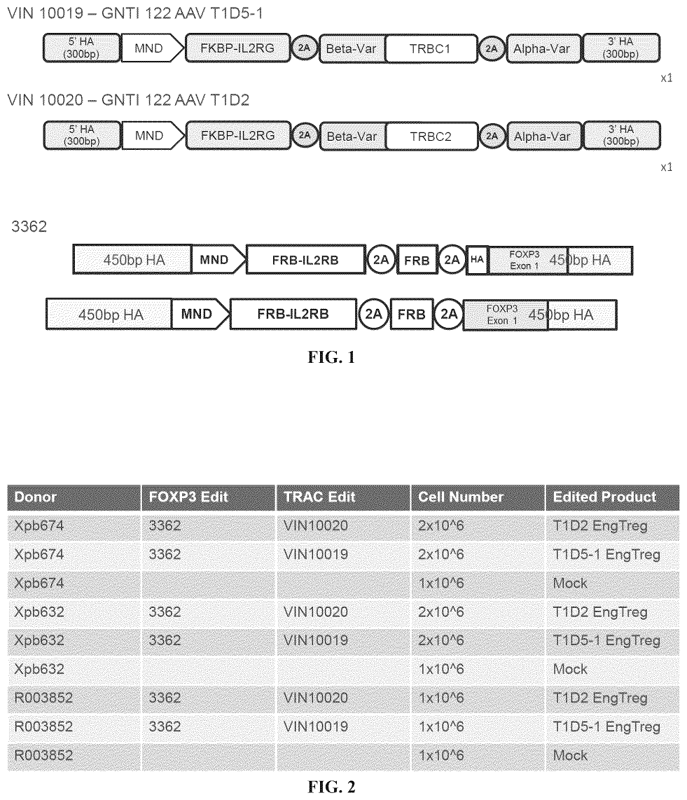

AAV donor constructs (polynucleotides) used for dual-editing are shown in FIG. 1. Using CRISPR-based editing, CD4+ T cells isolated from 2 subjects with T1D (Xbp674, Xbp632) or a healthy control donor (R003852; FIG. 2), were edited. To generate hT1D5-1-expressing EngTregs, cells were dual-edited with both the VIN 10019-Genti 122 AAV T1D5-1 donor and 3362 AAV donor. To generate hT1D2-expressing EngTregs, cells were dual-edited with both the VIN 10020-Genti 122 AAV T1D2 donor and 3362 AAV donor.

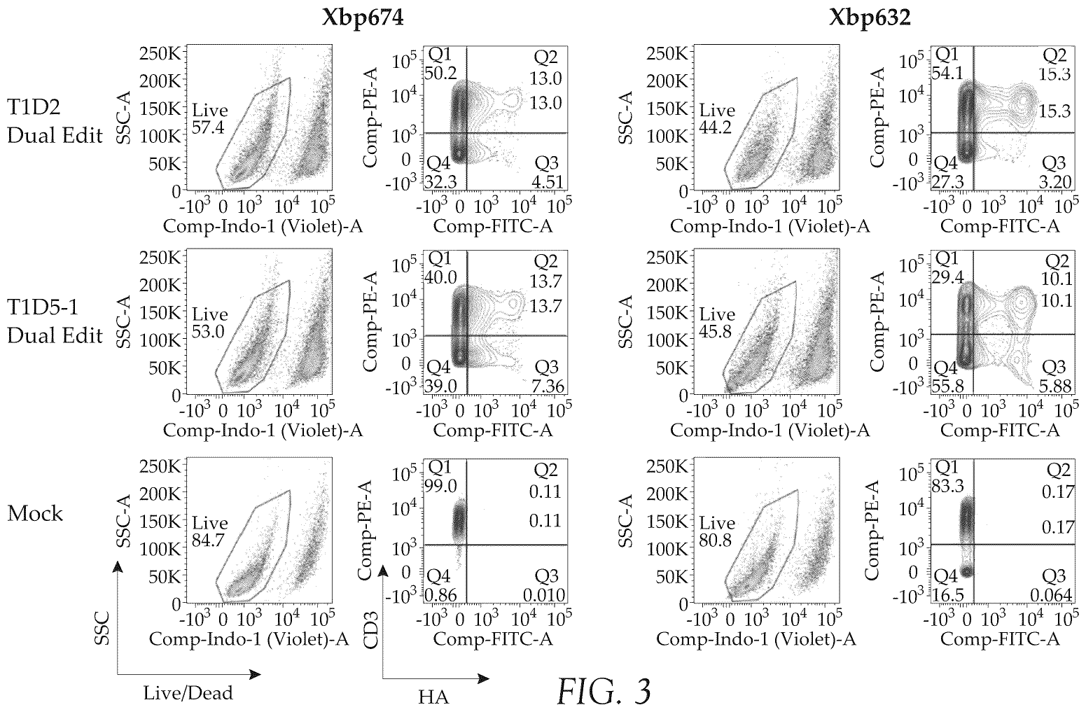

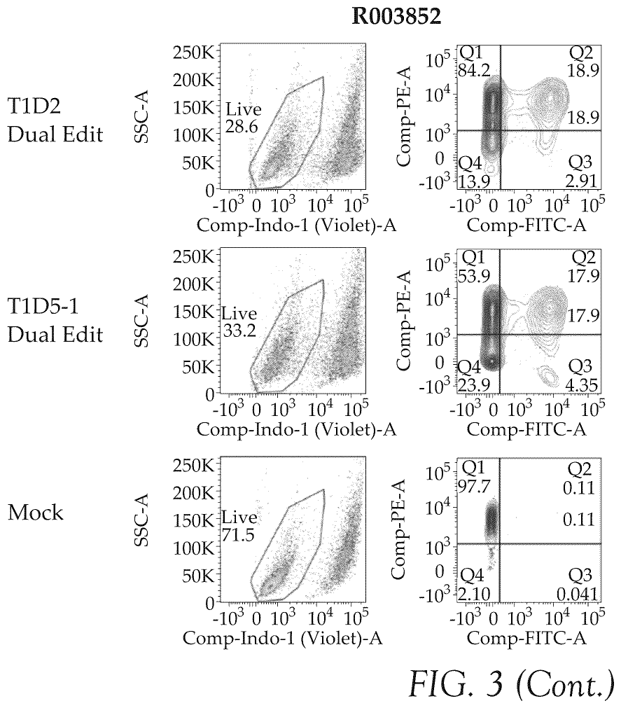

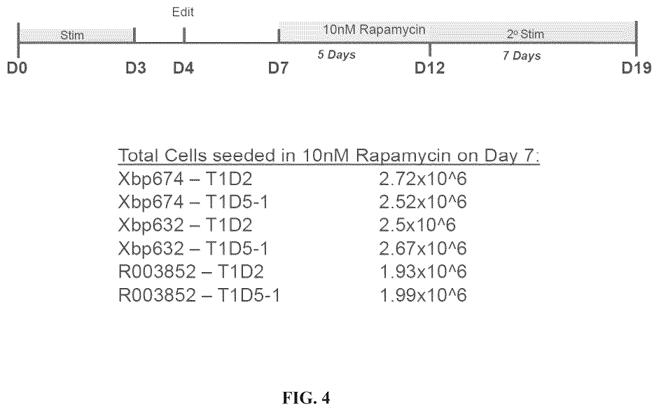

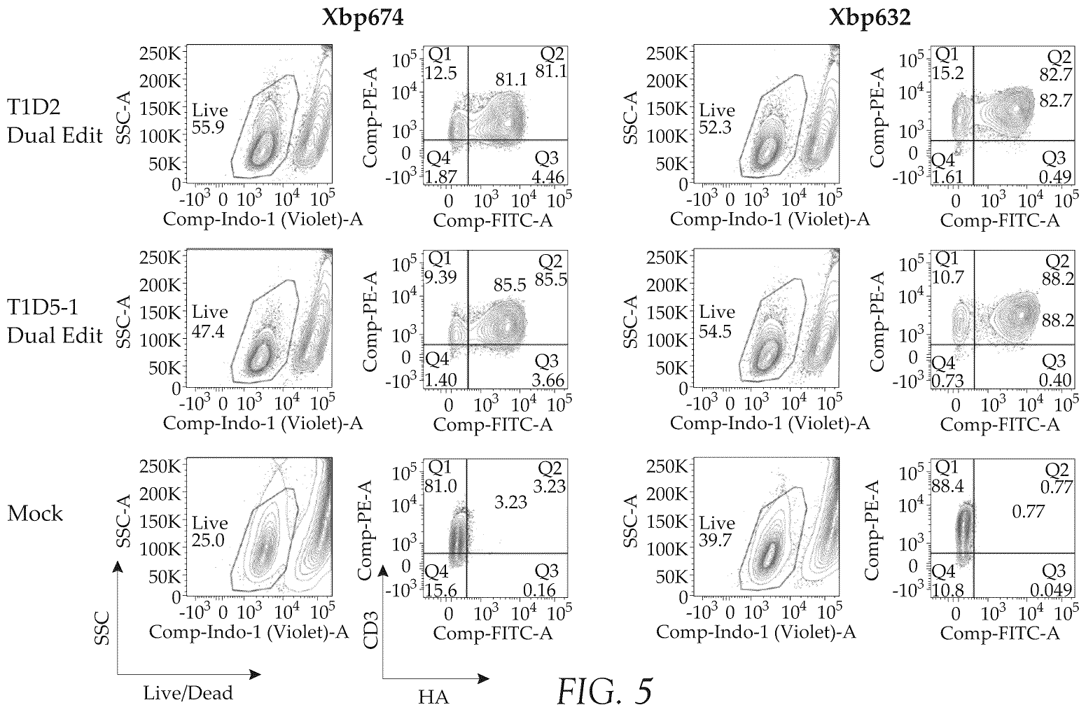

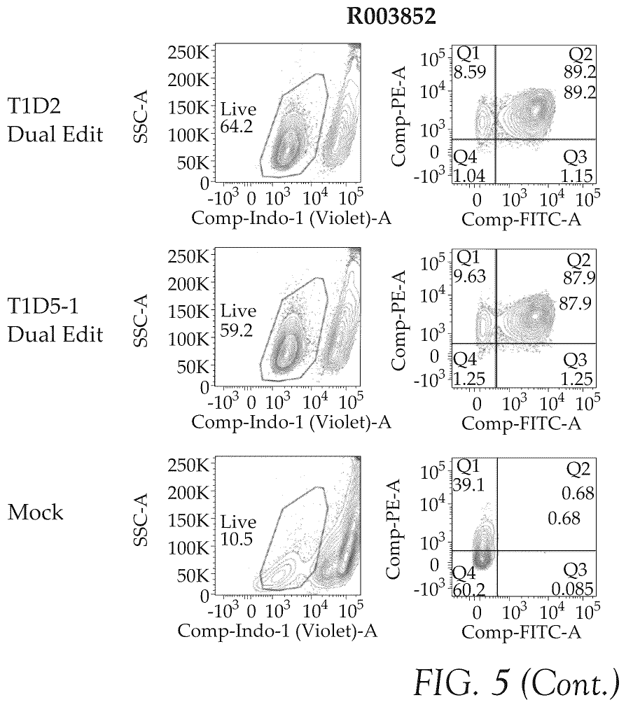

Initial FOXP3/TRAC dual-editing rates ranging from 10.1-18.9% were achieved (FIG. 3). By culturing the dual-edited cells in dimerizer (Rapamycin) using the approach shown in FIG. 4, a dual-edited population was enriched to >81% purity with selective expansion of the dual-edited cell population. Measurements were done using flow cytometry analysis for FOXP3 and Islet Ag-specific TCR expression (FIG. 5 and FIG. 8A).

Enriched cells were successfully cryopreserved (see Table 9 which shows total number of cell products from each donor/TCR dual-edit that were cryopreserved), and subsequent functional analysis, post thaw, demonstrated that dual-edited Ag-specific T1D2 or T1D5-1 EngTregs strongly suppressed the proliferation of T1D2 or T1D5-1 Teff cells expressing a matched islet Ag-specific TCR, in response to either non-specific (CD3/CD28) or specific (IGRP305-324 peptide) TCR activation. The findings demonstrate a potent direct, Ag-specific, Teff suppression by EngTregs (FIGS. 6A and 6B). Further, a bystander suppression phenotype was observed. Specifically, Ag-specific T1D2 or T1D5-1 expressing EngTregs derived from T1D subjects efficiently suppress the proliferation of a pool of autologous Teff cells derived from the same T1D subjects activated in vitro using APCs (mDCs) pulsed with a pool of islet peptides derived from 4 major islet antigens, including IGRP, GAD65, PPI and ZNT8 (FIG. 6C).

TABLE 9

| Donor | Edit | Total cells |
|---|---|---|
| Xpb674 | 3362/T1D2 | 6.98E+06 |
| Xpb674 | 3362/T1D5-1 | 8.19E+06 |
| Xpb674 | Mock | 8.36E+06 |
| Xpb632 | 3362/T1D2 | 6.05E+06 |
| Xpb632 | 3362/T1D5-1 | 3.25E+06 |
| Xpb632 | Mock | 1.51E+07 |
| R003852 | 3362/T1D2 | 5.50E+06 |
| R003852 | 3362/T1D5-1 | 5.50E+06 |
| R003852 | Mock | 1.65E+07 |

Also, in this polyclonal Teff assay, dual HDR edited T1D2 or T1D5-1 EngTregs outperformed T1D2 or T1D5-1 EngTregs generated via a combination of FOXP3 editing and lentiviral (LV) delivery of islet TCR (FIG. 7A and FIG. 7B) indicating that that the dual-editing platform leads to generation of Ag-specific EngTreg with optimal suppressive activity. These combined findings (FIG. 6C, FIG. 7A, and FIG. 7B) demonstrate that dual-HDR edited T1D2 or T1D5-1 EngTregs manifest robust bystander suppression of Teff from T1D subjects including Teff comprising a very broad range of TCR specificities.

Finally, dual-edited Ag-specific EngTregs exhibited a robust Treg immunophenotype including high level expression of FOXP3, CD25, CD39, CD73 and HLA-DR (FIGS. 8A and 8B).

These findings show that dual-HDR edited Ag-specific EngTregs have suppressive function that enable cell-based therapy for T1D.

### Example 2: Generation and Characterization of Immunosuppressive Capacity of Human T1D4 Expressing Dual-HDR Edited EngTregs for Use in T1D Therapy

A dual-editing strategy was performed using cells from two donors in which T1D4-EngTreg were prepared. Following a three-day CD3/CD28 stimulation, 5×10⁶ cells were edited using FOXP3 and TRAC guide RNAs followed by AAVs to knock in cassettes. A few days after editing, rapamycin enrichment was initiated to select for a dual positive, full CISC population.

An antibody detecting an expressed HA tag attached to the FOXP3 knock-in and TCRbeta variable region 5.1 was used to assess initial editing rates (FIG. 9). While the initial dual positive group ranged only from 2% to 6% of the population between the two donors, this population expanded to 80 to 85% when cultured in media supplemented with rapamycin (FIG. 10). As a secondary quantification of editing rates, a ddPCR assay was performed to detect homology directed repairs at each locus (FIG. 11).

Subsequent analysis of the final product demonstrated a robust Treg immunophenotype and secretome switch from pro-inflammatory to immunosuppressive cytokines. A phenotypic profile change in EngTregs was compared to mock edited population. An increase in expression of FoxP3, CD25 and CTLA4, mimicking a Treg phenotype was observed (FIG. 12). Cells were stimulated to observe differences in cytokine secretome. EngTregs had a marked decrease in production of inflammatory cytokines TNF-α, IFN-γ and IL-2 as compared to mock population (FIG. 13). An increase in the immunosuppressive cytokine TGF-β was observed in response to activation in the dual-edited cells compared to the mock (FIG. 14).

A functional ability of EngTregs to suppress T effector cells with a matched T1D4 TCR was examined. The T1D4 specific T effectors were cultured either alone, with mock edited cells or the dual-edited cells in addition to CD3/CD28 stimulatory beads or an antigen presenting cells with the T1D4 matched antigen, IGRP 241-260. When Teff cells were co cultured with mock edited population, about 40% suppression with CD3/CD28 stimulation was observed; however, with the antigen specific stim, little suppressive function (FIG. 15). The dual-edited engTregs showed a strong capacity to suppress matched antigen specific Teffs upon general stimulation and upon antigen specific stimulation. The dual-edited cell's ability to suppress proinflammatory cytokine secretion from the Teff was examined by coculturing with the antigen presenting cells and IGRP. There was a sharp decrease in the Teff secretion of TNF-α, IFN-γ and IL-2 when cocultured with the dual-edited compared to when cultured alone or with mock edited cells, indicating strong suppression by the EngTregs (FIG. 16).

A suppressive capacity of the dual-edited cells against Teff cells with a TCR of different specificity was examined. Preproinsulin (PPI) is an insulin precursor known to be a pancreatic antigen expanded in type one diabetes patients. While there was minimal suppressive action of EngTregs when cocultured with antigen presenting cells and PPI alone, it was observed that when IGRP was included in the coculture, a nearly 60% suppression of the PPI specific Teff cells was observed (FIG. 17).

Teff cytokine secretion when cultured with the PPI-specific stim alone was examined. Similar proinflammatory secretomes were observed when mock edited cells or dual-edited cells were added to culture. However, when cultured with the T1D4 specific antigen, IGRP, suppression of proinflammatory cytokine secretion by the dual-edited cells was not observed (FIG. 18).

### Example 3: Development and Characterization of GNTI-122, an Engineered Human Regulatory T Cell Therapy for Type 1 Diabetes

Type 1 diabetes (T1D) is an autoimmune disease caused by T lymphocyte-mediated killing of insulin-producing beta cells, which eventually leads to uncontrolled hyperglycemia and life-long dependence on continued insulin administration.

GNTI-122, an engineered T regulatory cell (Treg) for the treatment of T1D, is designed to protect islet cells, by homing to the pancreas and draining lymph nodes, and suppressing pathogenic effector T cells (Teff) through mechanisms including bystander suppression and infectious tolerance. GNTI-122 cells may be produced from autologous CD4+ T cells using nuclease-mediated gene editing to introduce (i) an MND promoter into the FOXP3 gene, downstream from the TSDR but upstream of the first coding exon, to stabilize FOXP3 expression by bypassing epigenetic transcriptional silencing due to TSDR methylation; (ii) a sequence encoding a pancreatic islet antigen-specific T cell receptor (isTCR) into the TRAC locus for antigen specificity; and (iii) sequences encoding components of a rapamycin-activated, synthetic IL-2 signaling receptor (CISC). Rapamycin-induced IL-2 signaling via CISC enables in vivo enrichment of GNTI-122 cells post-editing, and also aids in vivo cell engraftment.

The manufacturing process of autologous GNTI-122 engineered Tregs is shown in FIG. 20. The process began with isolation of PBMCs collected from leukapheresis procedure in hospital apheresis units, followed by magnetic enrichment of CD4+ T cells. CD4+ cells were then genetically modified using a targeted nuclease to cleave the cell genome at FOXP3 and TRAC loci, followed by knock-in of transgenes in adeno-associated virus (AAV) vectors by homology-directed repair. The frequency of GNTI-122 cells (expressing both the isTCR and FoxP3) was measured by flow cytometry, with FACS analysis showing proliferation of isTCR⁺FoxP3⁺ cells 3 days post-editing and just before cryopreservation (FIG. 21). The expanded cells were then cryopreserved for future infusion into subjects.

To evaluate the ability of CISC to facilitate GNTI-122 cell engraftment in vivo, edited GNTI-122 cells were administered to immunodeficient non-obese diabetic (NOD) mice lacking a functional Il2rg gene and mature B and T cells due to a Prkdc^{scid} mutation (NOD.Cg-Prkdc^{scid} I2rg^{tm1Whl}/SzJ (NSG™)). Mice were also administered rapamycin at one of a range of doses for 2 days pre-engraftment through 2 weeks post-engraftment (days 1-17). Expression of the CISC receptor by GNTI-122 cells, together with rapamycin administration, enabled selective expansion and enrichment of engineered Tregs in vivo in a dose-dependent manner (FIG. 22A).

To evaluate the effects of CISC stimulation on GNTI-122 cell population expansion in vitro, edited GNTI-122 and mock-engineered cells were cultured in the presence of rapamycin at a range of concentrations (FIG. 22B).

Additionally, GNTI-122 edited cells from two separate donors were cultured for 8 days in the presence of 10 nM rapamycin, with (FIG. 22D) and without (FIG. 22C) TCR stimulation by anti-CD3/CD28 beads. Without TCR stimulation, addition of rapamycin and CISC stimulation increased GNTI-122 survival, but the GNTI-122 population did not expand relative to baseline (FIG. 22C). In the presence of rapamycin and TCR stimulation, however, approximately 2-fold expansion of the GNTI-122 population was achieved (FIG. 22D). Finally, cells were also cultured with rapamycin at a range of concentrations from 0 to 30 nM, with TCR stimulation by anti-CD3/CD28 beads (FIG. 22E). The results shown in FIG. 22E demonstrate that GNTI-122 persisted and expanded with TCR stimulation in a rapamycin concentration-dependent manner.

In vitro analysis of regulatory marker expression, cytokine profile, and suppressive function demonstrated that GNTI-122 cells exhibit a Treg phenotype. Specifically, GNTI-122 cells exhibited Treg-associated markers, including CD25, CD27, CTLA-4, Eos, TNFRII, and TIGIT (FIGS. 23A and 23B), following thaw, a 3-day rest in culture, and staining by flow cytometry. This phenotype was consistent across distinct cell populations prepared from six independent cell donors. Additionally, GNTI-122 cells exhibited reduced inflammatory activity, as GNTI-122 cells (both alone or contacted with rapamycin) produced much lower amounts of inflammatory cytokines IFN-γ, TNF-α, and IL-2, relative to mock-engineered cells, when stimulated with PMA/ionomycin/monensin or anti-CD3/CD28 beads (FIG. 24A). Additionally, GNTI-122 cells expressed higher levels of Treg activation markers LAP and GARP following these stimulations, relative to mock-engineered cells (FIG. 24B). Functionally, GNTI-122 cells also inhibited the proliferation of FoxP3⁻ Teff cells expressing the same isTCR in an in vitro suppression assay (FIG. 24B).

GNTI-122 and mock-engineered cells were further assayed in vitro to evaluate suppressive capacity of EngTregs against distinct populations of Teff cells. GNTI-122 cells and mock-engineered were separately cocultured with both autologous Teff cells from donors with T1D, and monocyte-derived dendritic cells as antigen-presenting cells (APCs). In a first experiment to evaluate direct suppression, the Teff cells expressed the same TCR as GNTI-122 cells (T1D2), and APCs were loaded with the cognate IGRP peptide (FIG. 23C). In a second experiment to evaluate bystander suppression, the Teff cells expressed a different TCR specific to another T1D-associated antigen, preproinsulin (PPI) (FIG. 23D). In a third experiment, Teff cells specific to any of 9 different peptides of T1D-associated antigens were isolated to prepare a polyclonal Teff population, and APCs were loaded with a pool of those 9 cognate peptides (FIG. 23E). In each case, GNTI-122 cells exhibited strong direct (FIG. 23C) and bystander (FIG. 23D) suppression of monoclonal Teff cells, and robust suppression of polyclonal Teff cells (FIG. 23E).

The previously described GNTI-122 cells generated from T cells of healthy donors have been recapitulated with GNTI-122 cells generated from T cells of patients with T1D. Consistently, GNTI-122 generated from T cells of patients with T1D have similar initial dual editing rates, enrich to over 85% FOXP3+isTCR+, and gain a Treg-like phenotype. (FIGS. 23F-23H).

To assess the efficacy of the GNTI-122 engineering approach in vivo, a similar engineering approach was used to generate murine engineered Tregs (mEngTregs) by introduction of (i) MND promoter to allow stable FOXP3 expression; (ii) a murine pancreatic islet-specific TCR; and (iii) CISC to allow rapamycin-inducible IL-2 signaling, into murine cells. Diabetogenic splenocytes (T1D splenocytes) were intravenously injected into NSG™ mice, mEngTregs were intravenously injected 7 or 15 days post-T1D splenocyte administration, and blood glucose levels and time to T1D onset were monitored (FIG. 25A). While more than 50% of control mice developed T1D within 40 days of T1D splenocyte administration, administration of mEngTregs within 15 days substantially inhibited T1D development, and administration of mEngTregs within 7 days prevented T1D development entirely (FIG. 25B). Consistent with the delay in T1D onset achieved by administration of mEngTregs, blood glucose levels were better controlled in mice administered mEngTregs, compared to mice administered only T1D splenocytes (FIG. 25C). Evaluation of T cell abundance in multiple organs revealed that mEngTregs localized to the pancreas (FIG. 26A). Moreover, mEngTreg administration reduced both local and systemic Teff responses, as shown by reduced Teff memory cell abundance in the pancreas and spleen, respectively (FIG. 26B). Finally, mEngTregs inhibited insulitis induced by administration of T1D splenocytes, as histological analyses of pancreatic islets at day 43 post-T1D splenocyte administration revealed a greater proportion of “normal” islets in mice treated with mEngTregs, compared to control mice (FIG. 27A). This inhibition of insulinitis was corroborated by quantification of beta cell mass, which showed that beta cell mass in mice administered mEngTregs shortly (7 days) after T1D splenocyte administration resembled that of naïve mice, whereas beta cell mass was minimal in mice administered T1D splenocytes without mEngTregs (FIG. 27A). Finally, more insulin staining was observed in pancreata of mice administered mEngTregs than in mice administered only T1D splenocytes (FIG. 27C).

To show the robust persistence and efficacy of freshly prepared and cryopreserved mEngTregs, a similar mouse study was conducted where mEngTregs were administered 7 days after the diabetogenic splenocytes. Mice treated with both fresh or cryopreserved mEngTregs showed maintenance of normal blood glucose levels, and the mEngTregs in the pancreas. (FIG. 28)

These results indicate that the engineering approach overcomes the scaling limitations of alternative methods of preparing Treg cells (e.g., sorting human cells to isolate Tregs) by starting with more abundant T cell sources (e.g., bulk CD4+ T cells), and specifically enriching for edited cells with an engineered receptor that provides IL-2 proliferative signaling in the presence of rapamycin. Moreover, in vivo engraftment of such engineered cells may be supported by administration of rapamycin. Such engineered cells also display Treg-associated markers, cytokine production phenotypes, and suppressive functions in vitro. Finally, similarly engineered islet antigen-specific murine EngTregs suppressed ongoing pancreatic inflammation, preserving pancreatic islets and preventing T1D onset, demonstrating in vivo efficacy of this cell engineering approach.

### Example 4: Cellular and Suppressive Phenotypes of isTCR+FoxP3+ Dual-Edited EngTregs

CD4+ cells were thawed and stimulated with anti-CD3/CD28 Dynabeads in vitro (day 0). On day 1 post-thawing, cells were inoculated with a lentivirus encoding a T1D2, T1D5-1, or T1D4 TCR (day 1). On day 3 post-thaw, Dynabeads were removed. In parallel, artificial antigen-presenting cells were generated by transducing K562 cells with a lentivirus encoding an HLA-DR4 capable of presenting IGRP 305-324 or IGRP 241-260. On day 7 post-thaw, transduced CD4+ T cells were stimulated by addition of a given amount of cognate IGRP peptide in the presence of transduced K562 cells and culture overnight. On day 8 post-thaw, expression of activation-associated markers CD69, CD137, and CD154 (FIG. 29B). The results of these stimulations are shown in FIG. 29C. CD4+ T cells expressing each of T1D2, T1D4, and T1D5-1 TCRs upregulated functional markers CD154, CD69, and CD137 in a dose-dependent manner following stimulation with a cognate peptide (FIG. 29C). Lower concentrations of cognate peptide were required to achieve maximal surface marker expression in cells expressing T1D2 and T1D4, relative to cells expressing T1D5-1 (FIG. 29C).

On day 14, transduced CD4+ T cells were stimulated for 5 hours with cognate IGRP peptide in the presence of transduced K562 cells, and the production of cytokines IFN-γ and TNF-α to evaluate T cell activation (FIG. 29D). The results of these stimulations are shown in FIG. 29E. As with surface marker expression, CD4+ T cells expressing each of T1D2, T1D4, and T1D5-1 TCRs produced IFN-γ and TNF-α in a dose-dependent manner following stimulation with cognate peptide (FIG. 29E).

In another experiment, CD4+ T cells transduced with a lentivirus encoding T1D2 TCR or control TCR (ZNT266) were cultured in a 3:1 ratio with K562 cells pulsed with IGRP 305-324 peptide at a range of concentrations, as described in the preceding paragraph. After 20 hours of co-culture, expression of surface markers CD154 and CD137 were analyzed by flow cytometry, to quantify sensitivity of T1D2 TCR-expressing cells to cognate peptide IGRP 305-324. The results of this stimulation are shown in FIGS. 30A and 30B. Cells expressing T1D2 were substantially more sensitive to stimulation with cognate peptide IGRP 305-324 than cells expressing ZNT266 TCR, with CD154 expression having an EC₅₀ of 0.1-0.3 μg/mL IGRP 305-324 (FIG. 30C), and % CD137-expressing cells having an EC₅₀ of 0.03-0.1 μg/mL IGRP 305-324 (FIG. 30D).

In another experiment, CD4+ T cells transduced with a lentivirus encoding T1D2 TCR or control TCR (ZNT266) were cultured in a 3:1 ratio with K562 cells pulsed with 1 μg/mL IGRP 305-324 peptides, or variants containing an alanine substitution at one of 11 positions, as described in the preceding paragraphs. Peptide variants are shown in Table E4-1.

TABLE E4-1
IGRP 305-324 alanine-substituted peptides

| | Amino Acid Sequence | SEQ |
|---|---|---|
| Peptide | (substitution underlined) | ID NO: |
| IGRP 305 | QLYHFLQIPTHEEHLFYVLS | 231 |
| P1 | QLYAFLQIPTHEEHLFYVLS | 245 |
| P2 | QLYHALQIPTHEEHLFYVLS | 246 |
| P3 | QLYHFAQIPTHEEHLFYVLS | 247 |
| P4 | QLYHFLAIPTHEEHLFYVLS | 248 |
| P5 | QLYHELQAPTHEEHLFYVLS | 249 |
| P6 | QLYHFLQIATHEEHLFYVLS | 250 |
| P7 | QLYHFLQIPAHEEHLFYVLS | 251 |
| P8 | QLYHFLQIPTAEEHLFYVLS | 252 |
| P9 | QLYHFLQIPTHAEHLFYVLS | 253 |
| P10 | QLYHFLQIPTHEAHLFYVLS | 254 |
| P11 | QLYHFLQIPTHEEALFYVLS | 255 |

After 20 hours of co-culture, expression of surface markers CD154 and CD137 (among CD3+CD4+ cells) were analyzed by flow cytometry, to quantify the potential for off-target activation of cells expressing T1D2 TCR. The results of this stimulation are shown in FIGS. 31A and 31B. While the most activation was observed in culture with unmodified IGRP 305-324 peptide, some activation was observed in culture with peptides P1, P4, P7, and P11 (FIGS. 31A and 31B). Based on tolerance of T1D2 TCR to substitutions in these positions, a panel of potential off-target epitopes was produced, based on sequences present in pathogens of human relevance. Sequences of this panel are shown in Table E4-2.

TABLE E4-2
Potential off-target epitope peptides

| | | | | SEQ |
|---|---|---|---|---|
| | | | | ID |
| Peptide | Description | Source | Sequence | NO: |
| IGRP305_ | TPA: phosphopentomutase | Legionella sp. | SDSVLQIAAHEEHFG | 256 |
| 324_path | [Legionella sp.] | | | |
| 2 | | | | |
| IGRP305) | DUF4435 domain- | Bacillus | YDEVLQIPTHQENTQ | 257 |
| 324)path | containing protein | toyonensis | | |
| 5 | [Bacillus toyonensis] | | | |
| IGRP305_ | UDP-N- | Treponema sp. | AASFLHIPYFTHECD | 258 |
| 324_path | acetylglucosamine--N- | | | |
| 9 | acetylmuramyl- | | | |
| | (pentapeptide) | | | |
| | pyrophosphoryl- | | | |
| | undecaprenol N- | | | |
| | acetylglucosamine | | | |
| | transferase [Treponema | | | |
| | sp.] | | | |
| IGRP305_ | TPA: UDP-N- | Treponema sp. | AARFLHIPYYTHECD | 259 |
| 324_path | acetylglucosamine--N- | | | |
| 10 | acetylmuramyl- | | | |
| | (pentapeptide) | | | |
| | pyrophosphoryl- | | | |
| | undecaprenol N- | | | |
| | acetylglucosamine | | | |
| | transferase [Treponema | | | |
| | sp.] | | | |
| IGRP305_ | undecaprenyldiphospho- | Treponema sp. | AAKFLHIPVFTHECD | 260 |
| 324_path | muramoylpentapeptide | | | |
| 11 | beta-N- | | | |
| | acetylglucosaminyltrans | | | |
| | ferase [Treponema sp.] | | | |
| IGRP305_ | exodeoxyribonuclease V | Chlamydia caviae | CSPFLQIPSYEPIEY | 261 |
| 324_path | subunit beta [Chlamydia | | | |
| 12 | caviae] | | | |
| IGRP305_ | hypothetical protein | Pseudogymnoascus | SAMFLDIPTHPEEHS | 262 |
| 324_path | V495_01361 | sp. VKM F-4514 | | |
| 15 | [Pseudogymnoascus sp. | (FW-929) | | |
| | VKM F-4514 (FW-929)] | | | |
| IGRP305_ | hypothetical protein | Blastomyces | KYEFLPIPTYEEATS | 263 |
| 324_path | GX51_07420 [Blastomyces | parvus | | |
| 16 | parvus] | | | |
| IGRP305_ | hypothetical protein | Pneumocystis | FTDELNIPSYEELND | 264 |
| 324_path | T551_03071 | jirovecii RU7 | | |
| 17 | [Pneumocystis jirovecii | | | |
| | RU7] | | | |
| IGRP305_ | hypothetical protein | Encephalitozoon | LSEFLKIPNHEITLL | 265 |
| 324_path | KMI_05g09430 | hellem | | |
| 18 | [Encephalitozoon | | | |
| | hellem] | | | |
| IGRP305_ | hypothetical protein | Encephalitozoon | QQKFLKNIDTHEEAE | 267 |
| 324_path | ECU05_1220 | cuniculi GB-M1 | | |
| 19 | [Encephalitozoon | | | |
| | cuniculi GB-M1] | | | |
| IGRP305_ | WD domain, G-beta | Toxoplasma | ESLFLQLSTHTEASA | 268 |
| 324_path | repeat-containing | gondii ARI | | |
| 20 | protein [Toxoplasma | | | |
| | gondii ARI] | | | |
| IGRP305_ | ATP-dependent DNA | Legionella | DQLFLALPAHEERIS | 269 |
| 324_path | helicase [Legionella | lansingensis | | |
| 23 | lansingensis] | | | |

For all peptides listed in Table E4-2, the response of T1D2-expressing cells was similar to DMSO unstimulated control (FIGS. 31C and 31D). These results indicate that T1D2 does not recognize any of the predicted, potential off-target peptides derived from human pathogens.

### Example 5: A Phase 1/2, Open-Label Study of the Safety, Efficacy, and Cellular Kinetics of GNTI-122 in Adult and Paediatric Patients with Recently Diagnosed Type 1 Diabetes

This Example describes a Phase 1/2, open-label, multicentre study to evaluate the safety, efficacy, and cellular kinetics (CK) of GNTI-122 administered intravenously (IV) to adult and paediatric subjects with recently diagnosed type 1 diabetes (T1D). GNTI-122 is an autologous engineered Treg cell product containing two nucleic acids inserted into targeted loci by homology-directed repair. A first nucleic acid, inserted into the TRAC locus, encodes, under the control of an MND promoter: a first chemically inducible signaling complex component FKBP-IL2Rγ; a heterologous TCRβ chain; and a portion of a heterologous TCRα chain in-frame with a portion of the endogenous TCRα constant domain, such that the TCRα and TCRβ chains expressed from the TRAC locus form a TCR specific to a peptide of islet antigen IGRP. The second nucleic acid, inserted into the FOXP3 locus, encodes, under the control of an MND promoter: a second chemically inducible signaling complex component FRB-IL2Rβ; and a cytosolic FRB domain, both of which are in-frame with a portion of the endogenous FOXP3 coding sequence, such that the MND promoter inserted downstream from the Treg-specific demethylated region (TSDR) controls FoxP3 expression independently of the endogenous promoter and epigenetic regulation via TSDR methylation.

Primary objectives and associated endpoints of this study include: Phase 1. Objective: To assess the safety and tolerability of GNTI-122 with and without rapamycin in adult subjects with T1D. Endpoint: Cumulative adverse events/severe adverse events and clinically significant abnormalities in physical exams, vital signs, clinical laboratory measures, and other clinical assessments after the last adult subject has reached Week 12. Phase 2. Objective: To assess the efficacy of GNTI-122 with rapamycin in paediatric subjects with T1D. Endpoint: Change from baseline to Week 12, 24, and 52 in stimulated C-peptide area under curve (AUC) in paediatric subjects in Part B (Cohorts 3 and 4).

Other objectives and associated endpoints of this study include: Phases 1 and 2. Objective (Phase 1): To assess CK of GNTI-122 with and without rapamycin in adult subjects with T1D. Objective (Phase 2): To assess CK of GNTI-122 with rapamycin in paediatric subjects with T1D. Endpoint (Phases 1 and 2): Measurement of circulating EngTreg, with CK sampling at scheduled time points through Week 52. Phase 2. Objective: To assess the safety and tolerability of GNTI-122 with rapamycin in paediatric subjects with T1D. Endpoint: Cumulative AE/SAE and clinically significant abnormalities in physical exams, vital signs, clinical laboratory measures, and other clinical assessments for paediatric subjects in Part B (Cohorts 3 and 4) after the last subject has reached Week 12.

### Study Design

This is a Phase 1/2, open-label, multicentre study to evaluate the safety, efficacy, and cellular kinetics (CK) of GNTI-122 administered intravenously (IV) to adult and paediatric subjects with recently diagnosed type 1 diabetes (T1D).

Consented/assented adult and paediatric subjects with T1D undergo genetic testing for the DRB1*04:01 haplotype, due to the specific T cell receptor (TCR) reactivity of the GNTI-122 cell product; subjects who test positive for this allele may continue with the remainder of the Screening procedures.

Subjects who meet all eligibility criteria are entered into sequential dosing cohorts based on their age at Screening and receive study drug(s) as per the Schedules of Assessments (Table E5-4 and Table E5-5). Throughout this Example, the term “study drug” refers to GNTI-122 and rapamycin, unless otherwise specified.

### Safety

To maximise safety, all cohorts employ a sentinel subject approach: at least 7 days should elapse after the first subject in each cohort is dosed with GNTI-122 before dosing any other subject in that cohort.

Additionally, a staggered dosing approach between cohorts is used; all subjects in a preceding cohort must have completed their infusion of GNTI-122, and a safety review completed, before dosing any subjects in the subsequent cohort(s). For each safety review between cohorts (see FIG. 32), all available safety, tolerability, and CK data from the prior cohort(s) are evaluated to determine whether it is safe to proceed. This safety review does not take place until at least 7 days have elapsed since the last subject in the preceding cohort was dosed with GNTI-122. A minimum duration of 7 days was selected based on the finding that chimeric antigen receptor (CAR) T cell therapy-associated adverse events (AE) that may occur following infusion (such as Cytokine release syndrome [CRS] or neurologic syndromes such as CAR T cell-related encephalopathy syndrome [CRES] or immune effector cell-associated neurotoxicity syndrome [ICANS]) have a median onset of 2 days and 4 days post-infusion, respectively. This observation period is extended even further for the transition from the last adult cohort to the first paediatric cohort; in this case, the safety review does not take place until at least 28 days have elapsed since the last adult subject was infused with GNTI-122.

Exposure to rapamycin is minimised by using both an intermittent (approximately 1 week per month) dosing regimen as well as by targeting the lowest dose possible, as low levels are projected to be adequate to provide the necessary stimulatory signal for engraftment and persistence of GNTI-122 cells. The target trough range of rapamycin for approved indications is 4 to 20 ng/mL; the target trough level for this study is 4 ng/mL for each dosing cycle.

### Dose Selection

### GNTI-122—Adult Dosing

Precedence for the safe administration of polyclonal T regulatory cells (Tregs) has been previously established in the clinic across a dose range of 0.05 to 26×10⁸ cells, with no notable increase in safety risk observed with increasing doses.

The starting dose for GNTI-122 does not exceed 1×10⁸ cells, which is within the range safely tested with polyclonal Tregs. Of note, the islet antigen-specific TCR that has been engineered into GNTI-122, together with the knockout of the endogenous TCR, may further enhance the potential safety of the GNTI-122 product over that of the polyclonal Tregs that were previously administered to patients, which did not have TCR specificity.

A clinical dose has been selected for GNTI-122 based on the dose that was previously utilised for polyclonal Tregs, along with an added safety margin. This starting dose of GNTI-122 was selected based on the following considerations:
1. The precedence was established for safe administration of polyclonal Tregs in humans in a prior trial, across a multi-log dose range of 0.05 to 26×10⁸ cells, with no notable increase in safety risk observed with increasing doses of cells. For the GNTI-122 protocol, a starting dose of 1×10⁸ viable engineered Tregs (EngTregs) provides a safety margin approximately 25 times lower than the highest polyclonal Treg dose tested previously and carries the advantages of islet antigen specificity and tissue targeting that are engineered into GNTI-122.
2. The highest proposed dose of GNTI-122 (1×10⁹ cells) provides a safety margin at least 10-fold lower than the total number of natural endogenous Tregs in adult humans (estimated to be approximately 13×10⁹ Tregs).

### Rapamycin—Adult Dosing

Exposure-response models developed using in vitro data predict that rapamycin significantly enhances GNTI-122 engraftment and persistence at trough levels of rapamycin that are at the low end of those used for marketed indications.

For this study, the dose and schedule for rapamycin were determined by simulating rapamycin exposures that would provide interleukin-2 (IL-2) pathway signalling to GNTI-122 cells. A target trough concentration of approximately 4 ng/mL was shown to support GNTI-122 activation in vitro and engraftment in vivo.

### Pediatric Dosing Adjustments

Doses of GNTI-122 are adjusted for paediatric subjects based on mean pancreatic volume by age (Table E5-1) in order to provide equivalence to the highest adult dose tested in Phase 1 of the study. The proposed paediatric doses are dependent on first establishing the safety and tolerability of this dose in adults.

The rationale for using a pancreatic volume dose adjustment strategy is that GNTI-122 expresses a TCR specific for pancreatic antigen and is thus designed to traffic to the pancreas with limited circulation in the peripheral blood. Therefore, the aim of this dosing strategy is to ensure that approximately equivalent numbers of GNTI-122 cells engraft locally in the pancreas and its draining lymph nodes, where they are stimulated to mediate their immunoregulatory effects.

Rapamycin is administered to attain the protocol-targeted trough level (4 ng/mL) in subjects at each monthly dosing cycle through Week 52. Per the rapamycin package insert, subjects ≥13 years of age with body weight of at least 40 kg receive adult doses of rapamycin; all other subjects are to receive body surface area-based dosing. Based on the published literature for real-world rapamycin dosing data and modelling/simulations of rapamycin levels in paediatric subjects, a dose of 2 mg/day of oral rapamycin has been identified as the starting dose for subjects in this study ≥13 years of age with body weight of at least 40 kg (Table E5-2); a dose of 1.2 mg/m2/day has been identified as the starting dose for all other subjects.

### Study Cohorts

Part A: Eligible adult subjects (18 to <46 years of age at Screening) are enrolled into sequential dose-escalation cohorts to evaluate the safety, tolerability, and CK of GNTI-122. Cohorts 1a and 1b receive Dose 1 of GNTI-122 (1×10⁸ cells) and Cohorts 2a and 2b receive Dose 2 of GNTI-122 (1×10⁹ cells). Subjects in Cohorts 1b and 2b also receive concurrent rapamycin.
Part B: Eligible paediatric subjects (12 to <18 and 6 to <12 years of age) are enrolled in sequential, age-descending cohorts (Cohorts 3 and 4, respectively) to evaluate the efficacy, safety, tolerability, and CK of GNTI-122. To maximise safety, paediatric subjects do not receive an infusion of GNTI-122 until the study team has reviewed the cumulative safety, tolerability, and CK data for all adult subjects in Part A after at least 28 days have elapsed since the last adult in Part A was infused with GNTI-122. Paediatric subjects in Cohorts 3 and 4 receive Dose 2P of GNTI-122 (the adjusted paediatric dose to match adult Dose 2) plus rapamycin (adjusted for paediatric subjects) based on mean pancreatic volume by age (Table E5-1).
Part C: Eligible paediatric subjects (6 to <18 and 3 to <6 years of age) are enrolled in Expansion Cohorts (Cohorts 5 to 8 and Cohort 9, respectively) to collect additional data regarding the efficacy, safety, tolerability, and CK of GNTI-122. In these Expansion Cohorts, dose regimens of GNTI-122 and/or rapamycin may be reduced based on data from earlier cohorts.

Table E5-3 provides a summary of the cohorts and dose levels (see also Figure E5-1 for the study design).

Overall, approximately 60 subjects are planned: Part A, Adult subjects: n=12 (4 cohorts of 3 subjects each, 18 to <46 years of age). Part B, Paediatric subjects: n=16 (1 cohort of 8 subjects, 12 to <18 years of age; and 1 cohort of 8 subjects, 6 to <12 years of age). Part C, Paediatric subjects: n=32 (4 cohorts of 6 subjects each, 6 to <18 years of age; and 1 cohort of 8 subjects, 3 to <6 years of age).

Subjects in Part A or Part B who discontinue study participation (for non-study drug-related reasons) prior to completing the Week 12 visit may be replaced.

### Inclusion Criteria

1. Adult subject aged 18 to <46 years, with diagnosis of type 1 diabetes mellitus (T1D) meeting American Diabetes Association (ADA) criteria (e.g., fasting glucose >6.9 mmol/L or 2-h oral glucose tolerance test [OGTT] plasma glucose >11.0 mmol/L), diagnosed up to 78 weeks prior to consent; OR Paediatric subject aged as below, with diagnosis of T1D meeting ADA criteria, diagnosed up to 12 weeks prior to consent/assent. Cohort 3: 12 to <18 years of age. Cohort 4: 6 to <12 years of age. Cohorts 5-8: 6 to <18 years of age. Cohort 9: 3 to <6 years of age.
2. Adult subjects are able and willing to provide written, informed consent as approved by the independent ethics committee (IEC)/institutional review board (IRB). Adults must be able to consent directly; no other person or guardian may consent for them in this study.
3. Paediatric subjects are able and willing to provide assent, with a parent or legal guardian who provides written, informed consent as approved by the IEC/IRB.
4. No diabetic ketoacidosis (DKA) within 3 weeks prior to consent/assent or during Screening.
5. Subject requires and is on insulin therapy at the time of signing consent/assent. Note: Adult subjects should be insulin-dependent within the first 6 months after their diagnosis.
6. Subject is positive for the DRB1*04:01 (DR4) haplotype. By providing informed consent/assent for this study, all subjects are granting permission to have a genetic test for human leucocyte antigen (HLA) haplotype; this test is to be performed prior to continuing with other Screening procedures. Only subjects with the DRB1*04:01 (DR4) haplotype continue with Screening.
7. Subject has adequate vascular access to undergo leukapheresis with no known contraindications, including no known contraindications to central line placement (may be required for some subjects) and/or anaesthesia (as needed).
8. Female subjects of childbearing potential must have a negative serum pregnancy test at Screening and must agree to use contraception.
9. Male subjects of reproductive potential must agree to use contraception.
10. Subject has residual 3-cell function during Screening, defined as stimulated C-peptide >0.2 nmol/L after a mixed-meal tolerance test (MMTT). Note: if the stimulated C-peptide test was performed within 3 weeks of an episode of DKA and this criterion was not met, the stimulated C-peptide test may be repeated.
11. Adult subjects only: haemoglobin A1c (HgbA1c) is ≤9% during Screening. No restrictions on HgbA1c apply for paediatric subjects, given the short interval between diagnosis and Screening.
12. Body mass index at Screening is <36 (adult subjects) or <95th percentile for age (paediatric subjects); body weight is at least 15 kg for subjects 6 years of age and older at Screening, and at least 10 kg for subjects 3 to <6 years of age at Screening.
13. Renal function should be in the normal range at Screening, as per investigator judgement.
14. Other than T1D, subject is in good general health (per investigator judgement), based on medical history, physical examination, laboratory testing, and other evaluations during the Screening period.
15. Subject is willing and able to comply with study procedures and with the schedule of study visits.

### Production and Infusion of GNTI-122 and Follow-Up

To provide autologous T cells for GNTI-122 production, eligible subjects undergo leukapheresis at a qualified leukapheresis collection centre. The subject's leukapheresis sample is shipped to a production facility and processed to generate GNTI-122 product. GNTI-122 product is then be tested to verify product quality before release to the subject. Upon release, the GNTI-122 product is shipped to the study site for administration. The duration from leukapheresis collection to GNTI-122 shipment to the study site is expected to be approximately 8 to 10 weeks for each subject.

Subjects return to the study site to receive a single IV infusion of GNTI-122 (the day of infusion is designated as Day 0). The subject may be discharged from the study site after a minimum 4-hour observation period has elapsed and the investigator has assessed their health status.

After the Day 0 visit, subjects return to the study site for regularly scheduled follow-up visits as per the Schedules of Assessments (Table E5-4 and Table E5-5).

Each dose of GNTI-122 is created from autologous CD4+ T cells obtained by leukapheresis from the study subject. All subjects receive a single IV infusion of GNTI-122 on Day 0. Adult subjects receive a dose of 1×10⁸ cells (Dose 1) or 1×10⁹ cells (Dose 2), whereas paediatric subjects receive a dose (Dose 2P) based on mean pancreatic volume by age (see Table E5-1).

Intermittent low doses of oral rapamycin (in tablet or liquid form) are administered in monthly cycles as part of the study drug regimen for all subjects (except for subjects in Cohorts 1a and 2a, who receive GNTI-122 without rapamycin). The first dose of rapamycin is administered to subjects after completion of their GNTI-122 infusion on Day 0, as part of a once daily, 14-day course. After this initial dosing cycle, subjects take rapamycin once daily for 7 days every 4 weeks through Week 52. Trough levels are monitored to allow the investigator to make any needed adjustment to the subject's rapamycin dose for the next dosing cycle.

Adult subjects (Cohorts 1a, 1b, 2a, and 2b) also have a study visit at Day 3 for collection of additional blood samples. Paediatric subjects (Cohorts 3 to 9) do not have a visit at Day 3.

### Duration and End of Study

All subjects are assigned to receive a single IV infusion of autologous GNTI-122, with or without cycles of oral rapamycin. A subject is considered to have completed the main study if he/she has completed the assessments scheduled for the Week 76 visit or Early Termination (ET) visit, whichever comes first.

The end of the main study is defined as the date of the last visit of the last subject (at their Week 76 or ET visit). Week 76 was selected in order to allow longer-term assessment of GNTI-122 persistence, as well as durability of post-infusion clinical efficacy.

### Evaluation

Assessments are performed at the timepoints specified in the Schedules of Assessments (Table E5-4 and Table E5-5).

### Safety and Tolerability

Summaries of AEs and clinically significant abnormalities in physical exams, vital signs, laboratory tests, and other assessments/procedures are used to assess safety.

### Cellular Kinetics (Pharmacokinetics)

Peripheral blood samples are collected for CK to assess engraftment and persistence of EngTreg cells and the impact of rapamycin.

### Efficacy

Clinical measures of relevance to T1D outcomes, including glucose control, serial HgbA1c values, incidence of hypo- or hyperglycaemic episodes, changes in stimulated C-peptide levels, and daily insulin requirements are assessed.

### Pharmacodynamics

Peripheral blood samples are collected for evaluation of biomarkers, which may include (but are not limited to) serum cytokines and other inflammatory mediators, flow cytometric and epigenetic evaluation of peripheral blood mononuclear cells, and autoantibody levels; these data may also be assessed for correlation with clinical safety and efficacy outcomes.

### Immunogenicity

Peripheral blood samples are collected for the evaluation of pre-infusion and therapy-emergent antibodies to GNTI-122 EngTreg. These data are assessed for correlation with efficacy and safety outcomes.

### Patient-Reported Outcomes

Reported by subjects and/or their parent/legal guardian (as applicable): Diabetes Treatment Satisfaction Questionnaire (DTSQ) and DTSQ-Teen; Audit of Diabetes-Dependent Quality of Life (ADDQoL) and ADDQoL-Teen; and/or EuroQoL 5-Dimension (EQ-5D) and EQ-5D-Y.

### Statistical Analyses

Safety and efficacy data for adult and paediatric patients are listed, summarised, and analysed separately. Inferential statistics comparing the safety and/or efficacy between groups may be provided as needed using appropriate analysis methods. As adults are studied first, data analysis or interim analysis evaluates this population first.

Diabetes-related clinical assessments are performed in all subjects with T1D; however, the clinical outcomes data for the paediatric population (<18 years of age) are utilised for the primary efficacy endpoint and assessed separately from the data for adults (≥18 years of age). The area under the curve (AUC) of stimulated C-peptide by MMTT is summarised by time point along with change from baseline and is listed by age group and subject. Individual and summary plots for C-peptide are provided by treatment group over time. Summary statistics for C-peptide AUC and change from baseline are provided by treatment group and visit/time. Additionally, descriptive statistics for average daily dose of insulin are summarised over time by treatment group.

Unless otherwise specified, analysis are descriptive, based on listings and descriptive summaries. Continuous variables are summarised with the number of observations, mean, standard deviation, median, minimum, and maximum. Graphical summaries such as mean plot, spaghetti plot, box plot, or bar chart may be provided as well. Categorical variables are summarised with the number of observations and the numbers and percent from each category.

For all analysis sets, subjects are analysed according to the study procedure/treatment received.

The full analysis set includes all subjects who initiated any study procedures.

The safety analysis set includes all subjects that received any study drug.

The pharmacodynamic (PD) analysis set includes all subjects who received any study treatment and had available PD data and no protocol deviations with relevant impact on PD data.

TABLE E5-1
GNTI-122 Dose by Mean Pancreatic Volume Based on Age

| | Approximate Mean Pancreatic | Dose 2 of |
|---|---|---|
| | Volume in Healthy Adults | GNTI-122 |
| Group (Age, Years) | and Children (mL) | (# cells) |
| Adults (>=18 to <46) | 70 | 1 × 10⁹ |
| Pediatric (>=12 to <18) | 60 | 1 × 10⁹ |
| Pediatric (>=6 to <12) | 35 | 5 × 10⁸ |
| Pediatric (>=3 to <6) | 20 | 3 × 10⁸ |

TABLE E5-2
Rapamycin Dose to Support Engraftment

| | Rapamycin Dose to Support |
|---|---|
| Group | Initial GNTI-122 Engraftment |
| Subjects >=13 years of age | 2 mg/m² (oral) × 14 days |
| AND body weight >=40 kg | |
| Subjects <13 years of age | 1.2 mg/m² (oral) × 14 days |
| and/or body weight <40 kg | |

TABLE E5-3
Summary of Cohorts and Dose Levels

| | | | Number | Age at | GNTI- | |
|---|---|---|---|---|---|---|
| Trial | Study | Cohort | of | Screening | 122 | |
| Phase | Part | Number | Subjects | (Years) | Dose | Rapamycin |
| 1 | A (Adult) | 1a | 3 | 18 to <46 | 1 | No |
| | | 1b | 3 | | | Yes |
| | | 2a | 3 | | 2 | No |
| | | 2b | 3 | | | Yes |
| 2 | B (Pediatric) | 3 | 8 | 12 to <18 | 2P | Yes |
| | | 4 | 8 | 6 to <12 | 2P | |
| | C (Pediatric) | 5 | 6 | 6 to <18 | 2P | Yes |
| | | 6 | 6 | | | |
| | | 7 | 6 | | | |
| | | 8 | 6 | | | |
| | | 9 | 8 | 3 to <6 | 2P | Yes |

Dose 2P refers to the pediatric dose of GNTI-122 that has been adjusted to match the adult Dose 2 (see Table E5-1).

TABLE E5-4
Schedule of Assessments from Screening to Week 24

| | Study Visit | | | | | | | | | | | | | |
|---|---|---|---|---|---|---|---|---|---|---|---|---|---|---|
| | HLA | Main | | | | | | | | | | | | |
| | Screen- | Screen- | Leuka- | | | | | | | | | | | |
| | ing^{a} | ing^{a} | pheresis^{a} | BL | D0 | D3^{b} | D7 | D14 | W4 | W8 | W12 | W16 | W20 | W24 |
| | Study Day ± Window (days) | | | | | | | | | | | | | |
| | Up to 28 days prior | | | −7 to | | | | | 28 ± | 56 ± | 84 ± | 112 ± | 140 ± | 168 ± |
| | to leukapheresis | | — | D0 | 0 | ±1 | ±1 | ±2 | 2 | 2 | 3 | 3 | 3 | 3 |
| Informed | X | | | | | | | | | | | | | |
| Consent/Assent | | | | | | | | | | | | | | |
| for HLA typing | | | | | | | | | | | | | | |
| Sample | X | | | | | | | | | | | | | |
| collection for | | | | | | | | | | | | | | |
| HLA typing | | | | | | | | | | | | | | |
| Demographics | X | | | | | | | | | | | | | |
| Eligibility | X | | | | | | | | | | | | | |
| (inclusion/ | | | | | | | | | | | | | | |
| exclusion) | | | | | | | | | | | | | | |
| Medical/ | X | | | | | | | | | | | | | |
| surgical and | | | | | | | | | | | | | | |
| Family/social | | | | | | | | | | | | | | |
| histories | | | | | | | | | | | | | | |
| Body mass | X | | | | | | | | | | | | | |
| index | | | | | | | | | | | | | | |
| Height^{c} (cm) | X | | | | | | | | | | | Peds | | X^{c} |
| (without shoes) | | | | | | | | | | | | | | |
| Body weight | X | | X | | X | | | | X | | Peds | | | Peds |
| (kg) | | | | | | | | | | | | | | |
| Prior and | X | | X | | X | | X | X | X | X | X | X | X | X |
| concomitant | | | | | | | | | | | | | | |
| medications | | | | | | | | | | | | | | |
| Confirm HLA | | X | | | | | | | | | | | | |
| typing | | | | | | | | | | | | | | |
| Informed | | X | | | | | | | | | | | | |
| Consent/Assent | | | | | | | | | | | | | | |
| for full study | | | | | | | | | | | | | | |
| Vein assessment | | X | | | | | | | | | | | | |
| 12-lead ECG | | X | | | | | | | | | | | | |
| with site | | | | | | | | | | | | | | |
| interpretation | | | | | | | | | | | | | | |
| Provide CGM | | X | | | | | | | | | | | | |
| and apply initial | | | | | | | | | | | | | | |
| sensor | | | | | | | | | | | | | | |
| Optional genetic | | X^{d} | | | | | | | | | | | | |
| substudy | | | | | | | | | | | | | | |
| consent and | | | | | | | | | | | | | | |
| blood sample | | | | | | | | | | | | | | |
| collection | | | | | | | | | | | | | | |
| TSH, uric acid, | | X | | | | | | | | | | | | |
| LDH, CPK | | | | | | | | | | | | | | |
| T1D and other | | X | | | | | | | | | | | | |
| autoantibodies | | | | | | | | | | | | | | |
| Lipid panel | | X | | | | | | | | | | | | |
| (note if fasting) | | | | | | | | | | | | | | |
| Infectious | | X | | | | | | | | | | | | |
| disease testing^{e} | | | | | | | | | | | | | | |
| TB screening | | X | | | | | | | | | | | | |
| (QuantiFERON) | | | | | | | | | | | | | | |
| Chest x-rays if | | X | | | | | | | | | | | | |
| needed for | | | | | | | | | | | | | | |
| TB screening, | | | | | | | | | | | | | | |
| per local | | | | | | | | | | | | | | |
| guidelines | | | | | | | | | | | | | | |
| MMTT- | | X | | X | | | | | | | X | | | X |
| stimulated C- | | | | | | | | | | | | | | |
| peptide | | | | | | | | | | | | | | |
| measurement | | | | | | | | | | | | | | |
| Dispense | | X | X | | X | | X | X | X | X | X | X | X | X |
| Insulin Use | | | | | | | | | | | | | | |
| Diary with | | | | | | | | | | | | | | |
| instructions | | | | | | | | | | | | | | |
| Collect Insulin | | | X | | X | | X | X | X | X | X | X | X | X |
| Use Diary | | | | | | | | | | | | | | |
| Blood draw for | | X | | X | | X | X | X | X | | X | | | X |
| exploratory | | | | | | | | | | | | | | |
| biomarkers/Mo | | | | | | | | | | | | | | |
| A (PBMCs, | | | | | | | | | | | | | | |
| serum, plasma)^{f} | | | | | | | | | | | | | | |
| Pregnancy test | | S | U^{g} | | U^{g} | | | | U | U | U | U | U | U |
| Urinalysis (with | | X | | X | | | | | X | | | X | | |
| reflexive | | | | | | | | | | | | | | |
| microscopic test | | | | | | | | | | | | | | |
| if | | | | | | | | | | | | | | |
| abnormal) | | | | | | | | | | | | | | |
| Complete blood | | X | | X | | | | | X | | X | | X | |
| count with | | | | | | | | | | | | | | |
| differential | | | | | | | | | | | | | | |
| CD4 | | X | | | | | | | | | | | | |
| lymphocyte | | | | | | | | | | | | | | |
| count for | | | | | | | | | | | | | | |
| leukapheresis | | | | | | | | | | | | | | |
| calculations | | | | | | | | | | | | | | |
| Chemistries | | X | | X | | | | | X | | X | | X | |
| HgbA1c | | X | | X | | | | | | | X | | | X |
| Vital signs | | X | X | X | X^{h} | X | X | X | X | X | X | X | X | X |
| (HR, RR, BP, | | | | | | | | | | | | | | |
| body | | | | | | | | | | | | | | |
| temperature) | | | | | | | | | | | | | | |
| Tanner staging | | X | | X | | | | | | | X | | | X |
| (if <18 years of | | | | | | | | | | | | | | |
| age at visit)ⁱ | | | | | | | | | | | | | | |
| Physical exam | | F | D | D | D | D | D | D | D | D | D | D | D | D |
| (Full/F, or | | | | | | | | | | | | | | |
| Directed/D) | | | | | | | | | | | | | | |
| Leukapheresis | | | X | | | | | | | | | | | |
| Rapamycin | | | | X | | | | X | | X | X | X | X | X |
| levels | | | | | | | | | | | | | | |
| (except Cohorts | | | | | | | | | | | | | | |
| 1a and 2a) | | | | | | | | | | | | | | |
| Blood draw for | | | | X | | X | X | X | X | X | X | X | X | X |
| GNTI-122 CK | | | | | | | | | | | | | | |
| (molecular) and | | | | | | | | | | | | | | |
| cellular | | | | | | | | | | | | | | |
| persistence | | | | | | | | | | | | | | |
| (flow | | | | | | | | | | | | | | |
| cytometry) | | | | | | | | | | | | | | |
| Blood draw for | | | | X | | | | X | X | | X | | | X |
| immunogenicity | | | | | | | | | | | | | | |
| GNTI-122 IV | | | | | X | | | | | | | | | |
| infusion | | | | | | | | | | | | | | |
| 4-hour post- | | | | | X | | | | | | | | | |
| infusion | | | | | | | | | | | | | | |
| observation | | | | | | | | | | | | | | |
| period (subject | | | | | | | | | | | | | | |
| remains at study | | | | | | | | | | | | | | |
| site ) | | | | | | | | | | | | | | |
| Post-infusion | | | | | X | | | | | | | | | |
| blood draw | | | | | | | | | | | | | | |
| (serum/plasma) | | | | | | | | | | | | | | |
| Rapamycin | | | | | X | | | | | X | X | X | X | X |
| dosing cycle j | | | | | | | | | | | | | | |
| (except Cohorts | | | | | | | | | | | | | | |
| 1a and 2a) | | | | | | | | | | | | | | |
| Dispense | | | | | X | | | | X | X | X | X | X | X |
| rapamycin pack | | | | | | | | | | | | | | |
| (except Cohorts | | | | | | | | | | | | | | |
| 1a and 2a) | | | | | | | | | | | | | | |
| Rapamycin | | | | | | | X | | | X | X | X | X | X |
| accountability | | | | | | | | | | | | | | |
| (except Cohorts | | | | | | | | | | | | | | |
| 1a and 2a) | | | | | | | | | | | | | | |
| Patient- | | X | | | X | | | | X | | | | | X |
| Reported | | | | | | | | | | | | | | |
| Outcomes | | | | | | | | | | | | | | |
| (ADDQoL, | | | | | | | | | | | | | | |
| DTSQ, EQ- | | | | | | | | | | | | | | |
| 5D)^{k} | | | | | | | | | | | | | | |
| Adverse events | | X | X | X | X | X | X | X | X | X | X | X | X | X |
| collection | | | | | | | | | | | | | | |

ADDQoL: Audit of Diabetes-Dependent Quality of Life; BL: baseline; BP: blood pressure; CGM: continuous glucose monitoring; CK: cellular kinetics; CMV: cytomegalovirus; CPK: creatine phosphokinase; D: directed physical examination; D[#]: Day [#]; DTSQ: Diabetes Treatment Satisfaction Questionnaire; EBV: Epstein-Barr virus; ECG: electrocardiogram; EQ-5D: EuroQoL 5-Dimension; EQ-5D-Y: EuroQoL 5-Dimension-Youth; F: full physical examination; HBsA: hepatitis B surface antigen; Hep B: hepatitis B; Hep C: hepatitis C; HgbA1c: haemoglobin A1c; HLA: haplotype; HR: heart rate; HTLV: human T-lymphotropic virus; IV: intravenous; LDH: lactate dehydrogenase; MMTT: mixed-meal tolerance test; MoA: mechanism of action; PA: posterior-anterior; PBMCs: peripheral blood mononuclear cells; Peds: paediatric subjects; PRO: Patient-Reported Outcome; QD: once daily; RR: respiratory rate; S: serum; T1D: type 1 diabetes; TB: tuberculosis; TSH: thyroid-stimulating hormone; U: urine; W[#]: Week [#].
^{a}Only subjects with the DRB1*04:01 (DR4) haplotype will continue in Screening. The windows for HLA screening and main screening are with respect to leukapheresis. The window for baseline assessments is with respect to Day 0.
^{b}Day 3 assessments are to be performed only in adult subjects (Cohorts 1a, 2a, 1b, and 2b).
^{c}The height measurement should be repeated approximately every 6 months for any subject <18 years of age at the time of the study visit.
^{d}Blood sample for the optional genomic substudy may be collected at Screening or any other time during the study, as long as consent/assent is in place.
^{e}Infectious disease testing for HIV, HBsA, Hep B core antibody, Hep C viral RNA, syphilis, HTLV, EBV, and CMV.
^{f}See Laboratory Manual for full details and schedule of laboratory assessments.
^{g}Negative pregnancy test results should be documented prior to leukapheresis and prior to infusion on Day 0.
^{h}Vital signs should be collected both before and after study drug administration on Day 0.
ⁱTanner staging will be self-reported using scoring cards provided to the subject and their guardian.
^{j}On days of study visits (e.g., Day 0, Day 7, etc.), the daily rapamycin dose should be held until the subject is instructed to take the dose by site staff. The first dose of rapamycin will be administered after infusion of GNTI-122 on Day 0.
^{k}Paediatric subjects (<18 years of age) will complete youth versions of PROs as follows: ADDQoL-Teen and DTSQ-Teen (13 to 17 years of age) and EQ-5D- Youth (8 to 17 years of age).

TABLE E5-5
Schedule of Assessments from Weeks 28 to 76 (End of Main Study)

| | Study Visit (Weeks) | | | | | | | | | | | | |
|---|---|---|---|---|---|---|---|---|---|---|---|---|---|
| | | | | | | | | | | | | Early | |
| | 28 | 32 | 36 | 40 | 44 | 48 | 52 | 58 | 64 | 70 | 76 | Termination | Unscheduled |
| | Study Day ± Window (days) | | | | | | | | | | | | |
| | 196 ± | 224 ± | 252 ± | 280 ± | 308 ± | 336 ± | 364 ± | 406 ± | 448 ± | 490 ± | 532 ± | | |
| | 4 | 4 | 4 | 4 | 4 | 4 | 4 | 4 | 4 | 4 | 4 | | N/A |
| Lipid panel (note if fasting) | | | | | | | X | | | | | | |
| TB screening | | | | | | | X^{a} | | | | | | |
| QuantiFERON (per local | | | | | | | | | | | | | |
| guidelines) | | | | | | | | | | | | | |
| Chest x-rays if needed for | | | | | | | X^{a} | | | | | | |
| TB screen per local | | | | | | | | | | | | | |
| guidelines | | | | | | | | | | | | | |
| MMTT-stimulated C- | | | X | | | | X | | X | | X | X^{b} | |
| peptide measurement | | | | | | | | | | | | | |
| Dispense Insulin Use Diary | X | X | X | X | X | X | X | X | X | X | | | |
| with instructions | | | | | | | | | | | | | |
| Collect Insulin Use Diary | X | X | X | X | X | X | X | X | X | X | X | X | |
| Prior and concomitant | X | X | X | X | X | X | X | X | X | X | X | X | X |
| medications | | | | | | | | | | | | | |
| Blood draw for | | | X | | | | X | | | | X | | |
| exploratorybiomarkers/MoA | | | | | | | | | | | | | |
| (PBMCs, serum, plasma)^{c} | | | | | | | | | | | | | |
| Pregnancy test (subjects of | U | U | U | U | U | U | U | U | U | U | U | U^{d} | U⁴ |
| childbearing potential) | | | | | | | | | | | | | |
| Urinalysis (with reflexive | X | | | X | | | X | | | | X | | |
| microscopic test if | | | | | | | | | | | | | |
| abnormal) | | | | | | | | | | | | | |
| CBC with differential | X | | X | | X | | X | | X | | X | | |
| Chemistries | X | | X | | X | | | | X | | X | | |
| HgbA1c | | | X | | | X | | | X | | X | X⁵ | X^{e} |
| Vital signs (HR, RR, BP, | X | X | X | X | X | X | X | X | X | X | X | X | X |
| body temperature) | | | | | | | | | | | | | |
| Tanner staging (if <18 years | | | X | | | X | | X | | X | | | |
| of age at visit)^{f} | | | | | | | | | | | | | |
| Physical exam (Full/F, | D | D | D | D | D | D | F | D | D | D | D | D | D |
| Directed/D) | | | | | | | | | | | | | |
| Height (cm) (without shoes) | | | | | | | Peds | | | | Peds | Peds | Peds |
| Body weight (kg) | | | Peds | | | | All | | | | Peds | All | All |
| Rapamycin levels(except | X | X | X | X | X | X | X | | | | | X^{b} | |
| Cohorts 1a and 2a) | | | | | | | | | | | | | |
| Blood draw for GNTI-122 | X | | X | | | | X | | X | | X | X^{b} | |
| CK (molecular) and cellular | | | | | | | | | | | | | |
| persistence (flow cytometry) | | | | | | | | | | | | | |
| Blood draw for | | | X | | | | X | | | | X | X^{b} | |
| immunogenicity | | | | | | | | | | | | | |
| Dispense rapamycin pack | X | X | X | X | X | | | | | | | | |
| (except Cohorts 1a and 2a) | | | | | | | | | | | | | |
| Rapamycin dosing cycle g | X | X | X | X | X | X | X | | | | | | |
| (except Cohorts 1a and 2a) | | | | | | | | | | | | | |
| Rapamycin accountability | X | X | X | X | X | X | X | | | | | X | |
| (except Cohorts 1a and 2a) | | | | | | | | | | | | | |
| Patient-Reported Outcomes | | | X | | | | X | | | X | | | |
| (ADDQoL, DTSQ, EQ- | | | | | | | | | | | | | |
| 5D)^{h} | | | | | | | | | | | | | |
| Adverse events collection | X | X | X | X | X | X | X | X | X | X | X | X | X |

ADDQoL: Audit of Diabetes-Dependent Quality of Life; All: adult and paediatric subjects; BP: blood pressure; CBC: complete blood count; CK: cellular kinetics; D: directed physical examination; DTSQ: Diabetes Treatment Satisfaction Questionnaire; ET: early termination; EQ-5D: EuroQoL 5-Dimension; F: full physical examination; HgbA1c: haemoglobin A1c; HR: heart rate; MMTT: mixed-meal tolerance test; MoA: mechanism of action; N/A: not applicable; PA: posterior- anterior; PBMCs: peripheral blood mononuclear cells; Peds: paediatric subjects; PRO: Patient-Reported Outcome; RR: respiratory rate; TB: tuberculosis; U: urine; W: week.
^{a}Week 52 TB screen to be performed based on investigator judgement/local standard of care.
^{b}If prior test was ≥10 weeks prior.
^{c}Refer to the Laboratory Manual for full details and schedule of laboratory assessments.
^{d}The urine pregnancy test at the ET visit and any unscheduled visit should be performed if the previous result was obtained ≥4 weeks before the visit.
^{e}The HgbA1c test should be performed at the ET visit and any unscheduled visit if the previous test was completed ≥10 weeks before the visit.
^{f}Tanner staging will be self-reported using scoring cards provided to the subject and their guardian.
^{g}On days of study visits (e.g., Day 0, Day 7, etc.), the daily rapamycin dose should be held until the subject is instructed to take the rapamycin by site staff.
^{h}Paediatric subjects (<18 years of age) will complete youth versions of PROs as follows: ADDQoL-Teen and DTSQ-Teen (13 to 17 years of age) and EQ-5D- Youth (8 to 17 years of age).

### Example 6: Islet-Specific Engineered Treg Exhibit Robust Antigen-Specific and Bystander Immune Suppression in Type 1 Diabetes Models

### Introduction

Adoptive transfer of regulatory T cells (Treg) is therapeutic in Type 1 diabetes (T1D) mouse models. Notably, Treg specific for pancreatic islets are more potent than polyclonal Treg in preventing disease. However, the frequency of antigen-specific natural Treg is extremely low and ex vivo expansion may destabilize Treg leading to an effector phenotype. Disclosed herein are durable, antigen-specific engineered (Eng) Treg derived from primary human CD4+ T cells by combining FOXP3 homology-directed repair editing and lentiviral TCR delivery. Using TCRs from clonally expanded CD4+ T cells in T1D, islet-specific EngTregs that suppressed effector T cell (Teff) proliferation and cytokine production were generated. EngTregs suppressed Teff recognizing the same islet antigen in addition to bystander Teff recognizing other islet antigens via production of soluble mediators and both direct and indirect mechanisms. Adoptively transferred murine islet-specific EngTregs homed to the pancreas and blocked diabetes triggered by islet-specific Teff or diabetogenic polyclonal Teff in recipient mice. These data demonstrated the use of antigen-specific EngTregs as a targeted therapy to treat or prevent T1D.

T1D is an organ-specific autoimmune disease where autoreactive T cells target insulin-producing beta cells in the pancreatic islets resulting in a severe loss of endogenous insulin production (1, 2). Regulatory T cells (Treg), characterized by expression of the forkhead box transcription factor FoxP3, are important for maintaining peripheral tolerance and preventing excessive immune responses and autoimmunity. In humans, loss-of-function mutations in the FOXP3 gene leads to Treg defects resulting in a severe multi-organ autoimmune and inflammatory disorder referred to as immune dysfunction, polyendocrinopathy, enteropathy, X-linked (IPEX) syndrome. Among the wide range of autoimmune disorders in IPEX is early onset of T1D, demonstrating a key role of FOXP3+ Treg in maintaining islet-specific tolerance (1, 3). Several studies have suggested that reduced Treg number or impaired Treg function could be central to the pathogenesis of T1D (1, 2, 4, 5). Consequently, increasing the number or functional activity of Treg has become a major candidate strategy for therapeutic intervention to treat and prevent the disease (6, 7).

The therapeutic potential of Treg has been shown in various preclinical models of organ transplantation and autoimmune diseases (8). While adoptive transfer of expanded polyclonal Treg has shown clinical activity (8), it has been demonstrated that antigen-specific Treg are more efficacious than polyclonal Treg in numerous preclinical studies including T1D, multiple sclerosis, colitis, rheumatoid arthritis, and transplantation (9-15). For example, Treg specific for pancreatic islet antigens were more effective than polyclonal Treg in preventing T1D progression in murine models of T1D, and even reversed disease (9, 16, 17). Moreover, polyclonal Treg have multiple specificities and may lead to global immunosuppression (18). In contrast, antigen-specific Treg accumulate in target tissues and local lymphoid compartments where antigen presentation takes place, reducing the risk of off-target immunosuppression and making them both more efficacious and safer than polyclonal Treg for adoptive cell therapy.

Circulating Treg constitute only 1-2% of peripheral blood lymphocytes in humans (19-22) and the frequency of islet antigen-specific Treg in the blood is much lower. Isolating such rare cells is difficult and successfully expanding them to a clinically relevant number has not been reported to date. These challenges have motivated investigators to develop antigen-specific Treg through the transduction of TCRs with known specificities into Treg (8). TCR-transduced Treg selectively localize to the targeted tissue and can exert antigen-specific and bystander suppression (11, 13, 14, 23). However, as a therapeutic application, this approach has limitations due to the overall scarcity of Treg in the blood. Additionally, a fraction of Treg found in the blood are unstable under autoimmune inflammatory conditions (24-27) leading to concerns that extensive expansion may lead to loss of FOXP3 expression and reversion to an effector phenotype (8, 28, 29).

A gene editing approach designed to enforce FOXP3 expression in primary CD4⁺ T cells is disclosed herein (30). Introduction of a strong promoter element, MND, into the endogenous FOXP3 locus by homology directed repair (HDR)-mediated gene editing, mediated stable FOXP3 expression in human CD4+ T cells, resulting in robust production of engineered cells with Treg phenotype and suppressive function (EngTregs). As disclosed herein, this novel therapeutic platform was significantly expanded by combining FOXP3 gene editing with human TCR gene transfer to generate antigen-specific EngTregs from primary conventional CD4+ T cells. As disclosed herein, the capacity of these antigen-specific cell products to suppress both direct and bystander Teff responses via a variety of mechanisms in vitro and in vivo was demonstrated.

### Results

### Generation of Islet-Specific EngTregs by FOXP3 HDR-Editing and LV TCR Transduction

Human islet-specific EngTregs were generated using lentiviral vectors (LV) encoding islet-specific TCRs in conjunction with an approach to induce FOXP3 expression using HDR-based gene editing disclosed herein (30). Table E6-1 shows the six different islet-specific TCRs used in this study, derived from Teff isolated from individuals with T1D.

TABLE E6-1
Islet-specific TCRs

| | Islet | Epitope | | SEQ ID |
|---|---|---|---|---|
| TCR | antigen | location | Epitope sequence | NO: |
| T1D2 | IGRP | 305-324 | QLYHFLQIPTHEEHLFYVLS | 231 |
| T1D4 | IGRP | 241-260 | KWCANPDWIHIDTTPFAGLV | 232 |
| T1D5-1 | IGRP | 305-324 | QLYHFLQIPTHEEHLFYVLS | 231 |
| T1D5-2 | IGRP | 305-324 | QLYHFLQIPTHEEHLFYVLS | 231 |
| 4.13 | GAD65 | 553-572 | KVNFFRMVISNPAATHQDID | 233 |
| GAD265 | GAD65 | 265-284 | KGMAALPRLIAFTSEHSHFS | 234 |
| PPI76 | Preproinsulin | 76-90 | SLQPLALEGSLQKRG | 235 |

Notably all are HLA-DR0401 restricted and targeted distinct antigens; three recognized islet-specific glucose-6-phosphatase-related protein (IGRP), two recognized glutamic acid decarboxylase (GAD65) and one recognized pre-proinsulin (PPI) (31) and unpublished data). Importantly, these TCR specificities enabled assess to suppression of Teff responses by islet-specific Treg in a number of scenarios including: Treg and Teff having TCRs restricted to the same peptide-MHC complex; Treg and Teff having TCR restricted to different peptides within the same antigen; and Treg and Teff having TCRs with different antigen specificities. For each TCR, an expression cassette for the alpha and beta chain variable regions was cloned into a lentiviral backbone, and included the murine TCR constant region to ensure specificity of pairing between the transgenic TCR chains and permit antibody detection of the exogenous TCR (FIG. 33E, FIG. 33F). Antigen specificity of LV TCR transduced T cells was confirmed using a dye-based proliferation assay with proliferation occurring only in the presence of cognate peptide FIG. 33G). LV encoding islet-specific TCRs were next used to generate islet-specific engineered Treg (islet-specific EngTregs) as outlined in FIG. 33A. In brief, primary human CD4+ T cells were transduced with LV encoding islet-specific TCR after 24 h activation with CD3/CD28 beads. Two days after LV transduction, HDR editing of the FOXP3 locus was performed using CRISPR/Cas9 and an AAV6 donor template as described previously (30). As part of this donor cassette, a cis-linked, truncated LNGFR coding sequence (cytoplasmic domain deleted) (32) was introduced within exon 1 and separated by a P2A sequence to enable ribosomal skipping during translation (FIG. 33B). Inclusion of LNGFR allowed tracking and enrichment of the edited cells. Of the resulting transduced and edited T cells, 25-40% co-expressed intracellular FOXP3 and surface LNGFR, 70-95% of which expressed the transduced islet-specific TCR (FIG. 33C). In addition, transduced and edited cells were CD25⁺ CD127⁻ and upregulated CTLA-4 and ICOS expression, consistent with a Treg-like phenotype (30, 33-35). In the following study, these cells are referred to as islet-specific EngTregs.

### Islet-Specific EngTregs Exhibit Antigen-Specific Suppression of Teff Proliferation and Cytokine Production

To evaluate the suppressive function of islet-specific EngTregs, their effect was assessed on the proliferation of autologous Teff expressing the same islet-specific TCR in an in vitro suppression assay. Islet-specific EngTregs were enriched using LNGFR antibody affinity beads to greater than 85% purity (FIG. 33D); autologous Teff were prepared by transducing primary human CD4+ T cells with LV expressing the same islet TCR (FIG. 34E). Controls were untransduced EngTregs expressing endogenous polyclonal TCRs (henceforth referred to as poly EngTregs), and LV TCR-transduced T cells that were LNGFR−(non-binding fraction during LNGFR affinity bead enrichment; FIG. 33D), henceforth referred to as islet-specific LNGFR− T cells. Islet-specific EngTregs were co-cultured with cell trace violet (CTV)-labeled Teff in the presence of CD3/CD28 beads with CTV dilution used as a measure of Teff proliferation (FIG. 34A, FIG. 34B). Suppressive capacity was tested for the following islet-specific TCRs: T1D5-2 TCR specific for IGRP₃₀₅₋₃₂₄; PPI76 TCR specific for PPI₇₆₋₉₀; and GAD265 TCR specific for GAD65₂₆₅₋₂₈₄. It was confirmed that islet-specific EngTregs were able to suppress CD3/CD28 bead-induced Teff proliferation to similar levels as poly EngTregs (FIG. 34B, FIG. 34C). In contrast, islet-specific LNGFR− T cells had no effect on CD3/CD28 bead-induced Teff proliferation, demonstrating that the suppressive capacity was derived from FOXP3 editing (FIG. 34B, FIG. 34C). It was investigated whether the islet-specific EngTregs suppressed Teff proliferation in an antigen-specific manner by culturing in the presence of cognate peptide and APC. It was found that islet-specific EngTregs significantly suppressed antigen-induced Teff proliferation whereas poly EngTregs and islet-specific LNGFR− T cells did not (FIG. 34B, FIG. 34D). Notably, similar results were observed for all three islet-specific TCRs (FIG. 34B, FIG. 34C, FIG. 34D). In addition, we performed a Treg:Teff titration experiment directly comparing the suppressive activity of islet-specific EngTregs and poly EngTregs. We used islet-specific EngTregs expressing the T1D4 TCR 140 specific for IGRP₂₄₁₋₂₆₀ and suppression was assessed, in parallel, for both CD3/CD28-induced and antigen-induced Teff proliferation. This experiment demonstrated that islet-specific EngTregs are more potent than poly EngTregs at suppressing antigen-induced Teff proliferation, but had comparable suppression for CD3/CD28-induced Teff proliferation (FIGS. 34F-34I)

Since Treg have been reported to also suppress cytokine production by Teff (36-39), it was examined whether islet-specific EngTregs also suppress Teff cytokine production. For this experiment, both EngTregs and Teff expressed the T1D5-2 TCR and were cocultured in the presence of cognate IGRP₃₀₅₋₃₂₄ peptide and APC. Teff production of TNFα, IL-2 and IFNγ was determined by intracellular cytokine staining. Islet-specific EngTregs significantly suppressed antigen-induced Teff production of TNFα, IL-2 and IFNγ compared to poly EngTregs or islet-specific LNGFR− T cells, both of which had no significant effect (FIG. 35A, FIG. 35B). In addition, islet-specific EngTregs also suppressed Teff expression of the early activation marker CD25 (FIG. 35A, FIG. 35C). Collectively, these results indicated that antigen-specific suppression required not only suppressive capacity derived from FOXP3 editing, but also specific TCRs that received antigen-stimulation. They also demonstrated that islet-specific EngTregs exhibited antigen-specific suppressive capacity with respect to both Teff proliferation and cytokine production.

### Islet-Specific EngTregs Manifest Antigen-Specific Bystander Suppression

Activation of Treg is antigen-specific. However, once activated, Treg have the ability to exert bystander suppression (8, 40). This characteristic is especially important in the context of treating autoimmunity, where autoreactivity targets multiple tissue antigens. To determine whether islet-specific EngTregs can exert bystander suppression, it was investigated whether islet-specific EngTregs expressing the T1D4 TCR were able to suppress Teff expressing the T1D5-2 TCR (FIG. 36A). Note that T1D4 and T1D5-2 recognized two different IGRP epitopes, IGRP₂₄₁₋₂₆₀ and IGRP₃₀₅₋₃₂₄, respectively. T1D4 islet-specific EngTregs were co-cultured with T1D5-2 Teff in the presence of APC pulsed with either the T1D5-2 cognate peptide (IGRP₃₀₅₋₃₂₄) alone, or with a mixture of IGRP₃₀₅₋₃₂₄ plus the T1D4 cognate peptide (IGRP₂₄₁₋₂₆₀). Control Treg included poly EngTregs and T1D5-2 islet-specific EngTregs. Importantly, TCR expression levels were equivalent for both T1D4 and T1D5-2 in edited cells (FIG. 36H) and all EngTregs, irrespective of TCR, exerted similar Teff suppression in response to CD3/CD28 bead stimulation (FIG. 36I, FIG. 36J). As expected, and consistent with FIG. 34A-FIG. 34D, T1D5-2 Teff proliferation was suppressed by the T1D5-2 islet-specific EngTregs in the presence of either the cognate peptide IGRP₃₀₅₋₃₂₄ alone or with both peptides (FIG. 36B, FIG. 36C). In contrast, T1D5-2 Teff proliferation was only suppressed by T1D4 islet-specific EngTregs when both IGRP₂₄₁₋₂₆₀ and IGRP₃₀₅₋₃₂₄ peptides were present (FIG. 36B, FIG. 36C), findings consistent with bystander suppression. In contrast, islet-specific LNGFR− T cells showed neither direct nor bystander suppression of Teff proliferation, although they were activated by their cognate peptides (data not shown). Importantly, the capacity for bystander suppression was not limited to EngTregs with IGRP-specific TCRs. Bystander suppression was also detected for EngTregs expressing the GAD265 TCR, which suppressed proliferation of T1D5-2 Teff when both GAD₂₆₅₋₂₈₄ and IGRP₃₀₅₋₃₂₄ peptides were present (FIG. 36D, FIG. 36E). Bystander suppression was not observed using poly EngTregs, although they did show comparable suppression as GAD265 islet-specific EngTregs on T1D5-2 Teff proliferation induced by CD3/CD28 beads (FIG. 36K, FIG. 36L). In parallel studies, bystander suppression was tested in the context of Teff cytokine production, again utilizing T1D4 islet-specific EngTregs and T1D5-2 Teff. Similar evidence of bystander suppression was observed fro: IGRP₃₀₅₋₃₂₄-specific cytokine production and CD25 expression by T1D5-2 Teff were inhibited by T1D4 islet-specific EngTregs only when its cognate peptide IGRP₂₄₁₋₂₆₀ was present in addition to IGRP₃₀₅₋₃₂₄ (FIGS. 36F-36Q). In contrast, cytokine production and CD25 expression by T1D5-2 Teff was suppressed by T1D5-2 EngTregs in the presence of IGRP₃₀₅₋₃₂₄ alone or in combination with IGRP₂₄₁₋₂₆₀ (FIGS. 36F-36Q). In summary, these combined findings showed that islet-specific EngTregs had the ability to provide bystander suppression that limited both Teff proliferation and cytokine production.

Islet Specific EngTregs Suppress Polyclonal Islet-Specific T Cells from T1D Subjects Across Multiple Specificities

An initial assessment of the ability of islet-specific EngTregs to suppress in an antigen specific manner utilized Teff that were themselves transduced with LV encoding TCRs. However, the ultimate therapeutic goal is to suppress polyclonal islet-specific T cells in individuals at risk or with T1D. Therefore, a strategy was designed to assess the activity of islet-specific EngTregs against endogenous islet-specific Teff derived from PBMC of T1D subjects. Using PBMC from T1D donors, a parallel approach was used to generate: a) monocyte-derived DC (mDC) for use as APC; b) polyclonal islet-specific Teff; and c) EngTregs (FIG. 37A). To obtain islet specific Teff, CD4⁺CD25⁻ cells were cultured with irradiated autologous APC and a pool of 9 islet-specific peptides for 12-14 days (FIG. 37A, FIGS. 37E-37G). Peptides were chosen that were derived from IGRP, GAD65, and PPI that were known to be presented on HLA-DR0401 and for which HLA Class II tetramers were available (31, 41-45). This approach enabled Teff enriched for a mixture of islet specificities to be obtained, determined by tetramer staining, from multiple individuals with T1D. A broad range of tetramer positive cell frequencies was observed across donors, and T cells specific to GAD₁₁₃₋₁₃₂ and IGRP₂₄₁₋₂₆₀ were detected at a greater frequency than other specificities (FIG. 37F, FIG. 37G).

In parallel, CD4+ T cells from the same T1D donors were used to generate autologous T1D2 islet-specific EngTregs and 4.13 islet-specific EngTregs, with TCRs restricted to IGRP₃₀₅₋₃₂₄ and GAD65₅₅₃₋₅₇₃, respectively. These peptides were present among the islet peptide pool used to stimulate the polyclonal Teff (FIGS. 37E-37G). In a control experiment to test antigen-independent suppressive capacity, autologous poly EngTregs, T1D2 islet-specific EngTregs, and 4.13 islet-specific EngTregs exhibited comparable suppression of CD3/CD28 triggered Teff proliferation (FIG. 37B, FIG. 37C). In the setting of antigen-stimulation, polyclonal islet Teff proliferated in the presence of mDC and a mixture of 9 islet peptides (FIG. 37B, FIG. 37D). Poly EngTregs and islet-specific LNGFR⁻ T cells regardless of their TCR did not mediate suppression of polyclonal islet enriched Teff Strikingly, proliferation of islet peptide-specific T1D Teff was specifically suppressed by both T1D2 islet-specific EngTregs and 4.13 islet-specific EngTregs (FIG. 37B, FIG. 37D). Superior suppressive capacity was confirmed for islet-specific EngTregs under islet-specific stimulation and that expanded natural/thymic Treg (tTreg) did not exert notable Teff suppression (FIGS. 37H-37J). Together, these findings directly demonstrated that islet-specific EngTregs generated from individuals with T1D exhibit the capacity to mediate both antigen-specific and bystander suppression of autologous, autoreactive, islet-specific Teff.

### EngTregs Utilize Both Contact-Dependent and -Independent Suppressive Mechanisms

Tregs mediate suppression via multiple mechanisms including expression of anti-inflammatory soluble mediators, inhibition of APC maturation and consumption of IL-2 (8, 46). These mechanisms may also used by human, islet-specific, EngTregs. To investigate contact-dependent and -independent mechanisms, a transwell-based assay was used to assess the role for soluble factors produced by EngTress (FIG. 38A) (47, 48). Polyclonal islet-specific Teff were generated from CD4⁺CD25⁻ T cells from T1D subjects as above and in FIGS. 38G-38I. In the upper transwell chamber, T1D2 islet-specific EngTregs were plated either alone or co-cultured with polyclonal islet-specific Teff, and in the lower chamber, polyclonal islet-specific Teff were plated. Peptide loaded mDC were plated in both chambers and cell numbers were kept equivalent between chambers (FIG. 38A). T1D2 islet-specific EngTregs plated without Teff in the upper chamber significantly suppressed the proliferation of polyclonal islet-specific Teff in the lower chamber (FIG. 38B left, FIG. 38I). Thus, islet-specific EngTregs can mediate contact-independent suppression, presumably via production of transwell permeable soluble factors. However, contact-independent suppression was incomplete and was lower than that the positive control where the islet-specific EngTregs and the polyclonal islet-specific Teff were in direct contact (FIG. 38B left, FIG. 38I). Further, cell proximity also impacted the experimental outcome. EngTreg preferentially suppressed Teff that in closest proximity. T1D2 islet-specific EngTreg in the upper chamber suppressed proliferation of upper chamber Teff but had no effect on lower chamber Teff when Teff were present in both chambers (FIG. 38B left, FIG. 38I).

To determine whether islet-specific EngTregs could inhibit APC maturation, the effect of T1D2 islet-specific EngTregs on APC expression of CD80 and CD86 was assessed. In this assay, autologous monocytes restricted to HLA-DR0401 were matured into DC and then co-cultured with T1D2 islet-specific EngTregs in the presence of its cognate peptide IGRP₃₀₅₋₃₂₄ for 2 days (FIG. 38C). T1D2 islet-specific EngTregs were able to suppress mDC activation as measured by reduced mDC expression of CD86 compared to DCs alone or T1D2 islet-specific LNGFR− T cells (FIG. 38D; FIG. 38J). However, in contrast to previous studies showing that Tregs can also inhibit APC expression of CD80 (49, 50), islet-specific EngTregs had no impact on CD80 expression (FIG. 38K). Similar results were observed for T1D4 islet-specific EngTregs and PPI76 islet-specific EngTregs with both demonstrating ability to suppress CD86 expression on mDC but having no effect on CD80 expression (FIGS. 38M-38P).

The potential contribution of IL-2 consumption on EngTreg-mediated suppression was investigated. In mice, Treg consumption of IL-2 leads to cytokine deprivation-mediated apoptosis of Teff (51). However, it remains unclear whether this mechanism is operative in human Tregs with several studies reporting that IL-2 depletion is not required for Treg suppressive capacity (46, 52). Here, whether EngTreg suppression could be reversed by excess IL-2 was investigated, and found that addition of exogenous IL-2 had no significant effect on suppression of polyclonal islet-specific Teff proliferation (FIG. 38E, FIG. 38F).

Islet-Specific EngTregs with Lower Functional Avidity Exhibit Superior Suppressive Activity

As part of the studies, it was observed that alternative IGRP-specific TCRs utilized in the studies exhibited different functional avidities. Therefore, these unique features were used as way to begin to explore the impact of TCR affinity on islet-specific EngTregs function. First, we compared T1D2, T1D4 and PPI76 TCR that exhibit comparable expression levels of mTCR (FIG. 33H and FIG. 39F) and different functional avidities (FIG. 39A). Although CD4+ T cells transduced with PPI76 and T1D4 reached the similar proliferation at maximum concentration of their cognate peptide, PPI76 showed higher functional avidity than T1D4, with more than 20% proliferation at 0.01 ug/ml, whereas CD4+ T cells transduced with T1D4 showed similar proliferation at 0.1 ug/ml, 10-fold higher concentration. T1D2 TCR showed the lowest functional avidity among the three TCRs (FIG. 39A). We performed a side-by-side comparison of the relative suppressive capacity of EngTregs expressing each of these islet-specific TCRs, which showed comparable mTCR expression (FIG. 39E, FIG. 39F). We measured proliferation of polyclonal islet-specific Teff in the presence of islet-specific peptides and mDC and EngTregs with T1D2, T1D4 or PPI76. The data were normalized by suppressive activity obtained from suppression assay set up in parallel using CD3/CD28 beads (FIG. 39G). This latter assay provided a baseline control for EngTreg function, as this activation method is not impacted by TCR avidity. Strikingly, T1D2 islet-specific EngTregs, which had the lowest functional avidity, showed the highest percent suppression, followed by T1D4 and then PPI76 islet-specific EngTregs (FIG. 39B; FIG. 39G). We then compared T1D2, T1D5-1 and T1D5-2, each of which recognize the same cognate peptide, IGRP₃₀₅₋₃₂₄, in the context of HLA-DR0401 (Table E6-1) (31). As shown in FIG. 39C, these TCRs exhibited different functional avidities in response to cognate peptide, as determined in a dose response experiment measuring cell proliferation, this was independent of mTCR expression (FIGS. 33E-33H): T1D5-2 had the highest functional avidity with about 70% proliferation at peptide concentration at 0.1 μg/ml; followed by T1D5-1, similar proliferation at 1.0 μg/ml; and T1D2, with the lowest functional avidity, with proliferation only at 3 μg/ml. Similarly, we measured suppressive capacity of EngTregs expressing T1D2, T1D5-1, or T1D5-2 on proliferation of polyclonal islet Teff in response to islet-specific peptides. Consistently, T1D2 islet-specific EngTregs, which had the lowest functional avidity, showed the highest percent suppression, followed by T1D5-1 and then T1D5-2 islet-specific EngTregs (FIG. 39D; FIG. 39J). Together, these data suggested that there was an inverse relationship between TCR functional avidity and antigen-specific Treg suppressive capacity.

### Generation and In Vitro Characterization of Murine Islet-Specific EngTregs

To evaluate the in vivo efficacy of islet-specific EngTregs, methods were established to generate murine islet-specific EngTregs and tested their in vitro functional activity. Similar to the method for generating human islet-specific EngTregs, we used a CRISPR-Cas9-based HDR gene-editing strategy to introduce the MND promoter into the first coding exon of Foxp3, and a truncated LNGFR coding sequence was introduced upstream of Foxp3 (FIG. 40A, FIG. 40B). NOD.Cg-Tg(TcraBDC2.5,TcrbBDC2.5)1Doi/DoiJ (NOD BDC2.5) transgenic mice were used as the source of CD4+ T cells as these mice express an islet-specific TCR and rapidly induce diabetes when transferred into non-diabetic NOD mice (53-56). For negative controls, mock-edited NOD BDC2.5 CD4+ T cells were used that were electroporated without RNP and cultured in media containing the AAV5 donor template. In contrast to mock-edited cells, NOD BDC2.5 CD4+ T cells treated using both RNP and AAV demonstrated sustained LNGFR expression. Column-based LNGFR affinity purification resulted in ˜75% LNGFR+ cells (FIG. 40C), referred to hereafter as BDC2.5 islet-specific EngTregs. Enriched BDC2.5 islet-specific EngTregs demonstrated increased expression of LNGFR, FOXP3 and CTLA-4, with similar or higher CD25 expression compared to mock-edited cells (FIG. 40D, FIG. 40E).

The ability of the BDC2.5 islet-specific EngTregs to suppress the proliferation of activated islet-specific NOD BDC2.5 CD4+ Teff cells (abbreviated here as islet-specific Teff) in an antigen-dependent manner in vitro was tested. As in the human studies, proliferation by CTV dilution was assessed, and compared the suppressive capacity of BDC2.5-EngTregs, BDC2.5-tTreg and mock-edited cells (FIG. 40F). Both BDC2.5-tTreg and BDC2.5 islet-specific EngTregs showed dose-dependent suppression of BDC2.5-CD4⁺ Teff proliferation in comparison to mock-edited cells (FIG. 40G, FIG. 40H). tTreg displayed slightly better in vitro suppressive function than EngTregs, possibly reflecting the impact of thymic tTreg selection and/or programming in comparison to Teff converted EngTregs.

### Islet-Specific EngTregs Traffic to the Pancreas, Prevent Diabetes, and Stably Persist In Vivo

Whether BDC2.5 islet-specific EngTregs could prevent diabetes in vivo using a BDC2.5-CD4⁺ Teff induced T1D model was determined. In this model, adoptive transfer of BDC2.5-CD4⁺ Teff into immunodeficient nonobese diabetic (NOD)-scid-IL2rγ^{NULL} (NSG) mice rapidly promotes diabetes development as measured by blood glucose analysis (57). One of two doses (5×10⁴ or 1×10⁵) of BDC2.5 islet-specific EngTregs, or 5×10⁴ BDC2.5-tTreg (CD4⁺CD25^{hi} cells, column enriched and activated to match EngTregs) or mock-edited control cells were mixed with 5×10⁴ BDC2.5-CD4⁺ Teff (1:1 or 1:2 Teff:Treg ratios) and injected into 8-10 week old male recipient NSG mice (FIG. 41A). After cell transfer, blood glucose levels were monitored for up to 49 days; mice were sacrificed if they developed diabetes (blood glucose ≥250 mg/dL for two consecutive days). All diabetes-free animals were euthanized on day 49 for tissue and cell analysis. Mice infused with either BDC2.5 islet-specific EngTregs or -tTreg were almost completely diabetes-free, whereas all mice receiving mock-edited control cells developed diabetes within 9-15 days post-Teff transfer (FIG. 41B). Both doses of islet specific EngTregs prevented diabetes development. Thus, BDC2.5 islet-specific EngTregs were as effective as BDC2.5-tTreg in suppressing diabetes onset in this T1D mouse model. Thus, BDC2.5 islet-specific EngTregs functioned similarly to BDC2.5-tTreg in suppressing diabetes onset in this T1D mouse model.

In order to be beneficial, therapeutic Treg must home to the target tissue(s) and persist, maintaining a stable phenotype. To determine whether the islet specific EngTregs homed and persisted in the pancreas, pancreatic lymphocytes were isolated on day 49 by enzymatic digestion and performed flow cytometry to detect donor BDC2.5-CD4⁺ T cells (TCRvβ4⁺) and assessed the expression of LNGFR and FOXP3 (FIG. 41C). TCRvβ4⁺ EngTregs and tTreg were both present in the pancreas of diabetes-free mice on day 49. LNGFR+ cells were detected only in animals that received EngTregs (FIG. 41C) and these islet-specific, LNGFR+ EngTregs (CD4⁺TCRvβ4⁺LNGFR+) maintained high-levels of FOXP3 expression. Specifically, LNGFR+ EngTregs expressed similar levels of intracellular FOXP3 as tTreg (CD4⁺TCRvβ4⁺FOXP3⁺ cells); CD4⁺TCRvβ4⁺FOXP3⁻ cells (representing residual Teff cells) within the Treg recipient cohort (FIG. 41C). Together, these data evidence that, like BDC2.5-tTreg, BDC2.5 islet-specific EngTregs home to the pancreas and maintain FOXP3⁺ expression despite the sustained presence of islet-specific Teff.

Multiple reports have shown that islet-specific tTreg but not polyclonal tTreg are effective in preventing diabetes in T1D mouse models (9, 10, 17). To ask whether this was also true for our EngTregs, polyclonal EngTregs and tTreg were generated from NOD mice using identical methods (FIG. 41D) and compared them directly with BDC2.5 islet-specific-EngTregs and -tTreg in the NOD T1D model. 1×10⁵ polyclonal-EngTregs, or -tTreg, or BDC2.5-EngTregs or -tTreg, or mock-edited control cells were mixed with 5×10⁴ BDC2.5-CD4⁺ Teff and injected into 8-10 week-old male NSG mice (FIG. 41E). Recipients were monitored for up to 49 days for diabetes development. Consistent with previous reports, polyclonal tTreg were minimally effective in preventing T1D development, with only ˜20%-of mice remaining diabetes-free. Similarly, only limited protection was observed in recipients of polyclonal EngTregs. In contrast, nearly all mice (˜95%) receiving BDC2.5 islet specific-EngTregs or -tTreg remained diabetes-free (FIG. 41E).

Next we sought to assess the bystander-suppressive capacity of islet specific EngTregs in vivo. To confer the diabetogenic TCR repertoire of NOD mice to NSG mice, 2.25×10⁶ unfractionated splenocytes derived from diabetic NOD donors were co-delivered along with 1×10⁵ BDC2.5 EngTregs into 11-week-old female NSG mice (FIG. 41F). Recipients were monitored for up to 33 days for diabetes development. Consistent with our in vitro data demonstrating that islet-specific human EngTregs are capable of broad bystander suppression, all mice receiving BDC2.5 EngTregs were protected from developing diabetes (FIG. 41G). Consistent with these observations, histologic examination of pancreata isolated from animals receiving only diabetogenic NOD splenocytes, revealed marked infiltration CD3+ mononuclear cells within the islets and the surrounding interstitial tissue (FIG. 41H, FIG. 41I), correlating with complete, or near complete, loss of insulin-staining b-cells. Co-delivery EngTregs substantially reduced the severity of lymphocytic insulitis resulting in preservation of islets and insulin expression approaching that present in untreated control animals. Taken together, these findings demonstrate a robust capacity for islet-specific EngTregs to prevent T1D development in vivo and, consistent to previous work using tTreg, they show that expression of an islet specific TCR markedly improves the potency of EngTregs.

### Discussion

As described herein, the ability of antigen specific T cells derived from PBMC followed by FOXP3 editing to function in an antigen specific manner was demonstrated (30). While technically feasible, this method had several limitations in the context of autoimmunity: T cells specific for self-antigens are rare in the peripheral blood and expansion to numbers and cell purities likely to be required for therapeutic application are difficult and time consuming. Hence, as disclosed herein, efforts were focused on combining efficient delivery of islet specific TCRs derived from T1D subjects ((31) and unpublished data) with HDR-gene editing. This combined approach results in a 10-fold increase in the number of engineered Tregs from the number of CD4+ T cells isolated from PBMC used as starting material (data not shown), and yields as many as 10⁹ EngTregs from 400 cc of blood, similar to the numbers obtained by expansion of tTreg used in clinical trials of polyclonal Treg (6). Importantly, a suppressive capacity of islet-specific EngTregs under antigen-specific stimulation was demonstrated which had not been seen with either polyclonal-EngTregs or tTreg. Accordingly, the study disclosed herein in a murine model of T1D also showed that islet antigen-specific EngTregs blocked diabetes triggered by islet-specific Teff, while polyclonal EngTregs failed to limit disease progression.

It has been suggested that Treg expressing TCRs that recognize tissue-specific peptides may preferentially accumulate in target tissues, where they can be activated by these autoantigens and mediate bystander suppression (58). Mouse studies disclosed herein showed that islet-specific EngTregs localized in the pancreas following adoptive transfer and effectively suppressed diabetes triggered by islet-specific Teff Given the possibility that polyclonal Treg can interfere with immune responses to pathogens, the ability to home to target tissues is likely critical for both efficient on-target immune suppression and for limiting the risk of impairing systemic immunity (8, 14). Further, in vitro data in human cells demonstrated that islet-specific EngTregs suppress bystander Teff with many different specificities. This breadth of bystander suppression is predicted to permit islet-specific EngTregs to locally suppress pathogenic Teff with multiple specificities including limiting Teff responses where the target autoantigens are unknown. Thus, the combination of the targeted homing and bystander suppressive capacity by EngTregs with islet-TCR likely provides a more efficient and safer strategy to treat and control autoimmune diabetes (59).

Functional studies of antigen specific human Treg is largely limited to in vitro suppression assays and it remains unclear whether these assays accurately predict in vivo function. While islet-specific EngTregs were demonstrated to mediate efficient suppressive activity on Teff in a model system utilizing Teff transduced with a relevant TCR, this system has limitations based upon testing Teff with a single specificity. Given the diversity of pathogenic autoreactive T cells in T1D, whether islet-specific EngTregs would also inhibit endogenous T1D-relevant Teff with a broad range of TCR specificities was investigated. The data provide a significant advance in this arena. Using a pool of antigen enriched, islet-specific Teff derived from T1D subjects (based upon stimulation with a broad panel of islet peptides across multiple antigens), it was demonstrated that the capacity to suppress polyclonal Teff populations using islet-specific EngTregs with single islet antigen specificity. These findings support the concept that islet-specific EngTregs can mediate antigen-specific and bystander suppression of autologous, islet-specific Teff present in T1D subjects.

Multiple mechanisms have been implicated in the suppression of CD4 T cells by Treg including modulation of costimulatory receptors on APC, production of soluble factors (such as generation of adenosine by conversion of ATP via CD39/CD73, IL-10, TGF-3 and IL-35) and consumption of IL-2 (8, 46, 60). Here, mechanisms whereby islet specific EngTregs function were explored taking advantage of an ability to assess suppression in an antigen specific manner using autologous T1D subject-derived, CD4 T effectors enriched for specificity to islet antigens. Using this approach, it was demonstrated that, while IL-2 consumption is not a driver of suppression in this setting, EngTregs can function to down-modulate APC activation. Further, the transwell-based analyses showed a contribution of both contact-independent and -dependent suppressive activity. The data support an important role for soluble EngTreg secreted factors in Teff suppression. Further, the loss of suppression of Teff in the lower wells, when EngTregs suppressed co-cultured Teff in the upper wells, indicates that these soluble factors are likely consumed by Teff in closest proximity. This finding differed from data reported by Kim et al. where IL-2 produced by neighboring effector T cells activated Treg, subsequently initiating contact-independent suppression of effectors in the adjacent well (23). However, our findings related to IL-2 consumption and the role of contact and non-contact suppression are consistent with other published studies using tTreg (37, 52, 60, 61). Additionally, our studies differ from others in our finding only modest changes in CD86 in APC in co-culture assays (49, 50, 52); possibly reflecting differences between murine and human Treg (46, 49, 51, 52) and/or the use of non-specific stimulation in the absence of APC, the use of different types of APC, or use of effectors with a single specificity as compared the polyclonal antigen-specific T cells evaluated in our studies (23, 48, 50). Lastly EngTregs have been derived from CD4 effector T cells and edited to constitutively express FOXP3 at relatively high levels and thus may have different functional characteristics than tTreg.

Notably, when comparing TCR with the same MHC-peptide restriction but with different functional avidities, we observed greater suppressive activity in EngTregs expressing a lower avidity TCR. Studies utilizing CAR or TRuC receptors in Tregs indicate that the character of the signal can play a significant role in Treg function. (50, 62). Studies in murine models using Tg TCR have suggested that Treg with high functional avidity are more potent (63, 64). However, a role of low affinity Treg has also been shown in a polyclonal NOD model (65). In that study, low affinity Treg were able to compete with high affinity Treg, accumulate in sites of inflammation and the combined presence of both low and high avidity Tregs gave greater protection from diabetes (65).

Studies with BDC2.5 TCR mice showed that low peptide doses induced significant expansion of FOXP3+ Treg via mTOR pathway and that adoptive transfer of low-Ag-expanded BDC2.5 T cells, with splenocytes from diabetic NOD mice, prevented diabetes in NOD-SCID recipients, whereas mice given splenocytes plus high-Ag-expanded BDC2.5 T cells developed diabetes (66, 67). Studies of transduced human Treg are limited. Low affinity Class I restricted TCR transduced into Treg confer potent antigen-specific suppressive activity and impede expansion of high avidity CD8+ T cells (68). A study of Treg expressing GAD 555-567 specific TCRs, 4.13 and R164 (low and high affinity, respectively), demonstrated the capacity of each TCR to confer regulatory function. However, R164 T cells exhibited greater suppression, suggesting an advantage for the high avidity TCR (69). In our study, we employed a polyclonal islet-specific Teff pool and EngTregs expressing equivalent levels of exogenous TCR with either distinct specificities or EngTregs expressing three alternative TCRs restricted to the same MHC-peptide complex. In both settings, the TCR with lowest functional avidity yielded the greatest suppression. Our findings may differ from previous work due to the type of Teff target, the culture conditions and/or the mechanism(s) required for suppression by EngTregs.

In summary, we describe an efficient strategy to generate antigen-specific EngTregs from primary CD4+ T cells via combining of FOXP3 HDR-editing and LV TCR transfer. We show that EngTregs expressing islet-TCRs can suppress both proliferation and cytokine production of antigen-specific and bystander effector Teff. Further, islet-specific EngTregs suppress autologous pathogenic polyclonal T cells expanded from PBMC of T1D patients. Consistent with these findings, adoptively transferred, islet-specific EngTregs accumulated in the pancreas and prevented diabetes triggered by islet-specific or polyclonal diabetic Teff in vivo in recipient mice. Taken together, these findings strongly support the future potential for antigen-specific EngTregs in treatment of T1D and, possibly, in other organ specific autoimmune or inflammatory disorders.

In summary, described herein is an efficient strategy to generate antigen-specific EngTregs from primary CD4+ T cells via combining of FOXP3 HDR-editing and LV TCR transfer. It was shown that EngTregs expressing islet-TCRs suppressed both proliferation and cytokine production of antigen-specific and bystander effector Teff. Further, islet-specific EngTregs suppressed autologous pathogenic polyclonal T cells expanded from PBMC of T1D patients. Consistent with these findings, adoptively transferred, islet-specific EngTregs selectively accumulated in the pancreas and prevented diabetes triggered by islet-specific Teff in vivo in recipient mice. Taken together, these findings strongly support the use of antigen-specific EngTregs in treatment of T1D and in other organ specific autoimmune or inflammatory disorders.

### Materials and Methods

### Study Design

The objective of this study was to test whether durable, antigen-specific EngTregs could be generated using a gene editing approach combining FOXP3 homology directed repair editing and lentiviral TCR delivery. The ability of human islet specific EngTregs to suppress Teff proliferation and cytokine production in the presence of the cognate vs. irrelevant antigens were assessed in vitro. The ability of murine islet-specific EngTregs to traffic to the pancreas, prevent diabetes, and stably persist in vivo were assessed in a T1D mouse model using BDC2.5-CD4+ Teff to induce disease. Investigators were not blinded to the treatment. Figure legends list the sample size, number of biological replicates, number of independent experiments and statistical method.

### Primary Human T Cells

Human PBMCs were obtained from the Benaroya Research Institute (BRI) Registry and Repository were approved by BRI's Institutional Review Board (IRB #07109-588). Healthy control subjects had no personal or family history of autoimmune disease. Both healthy control and T1D subjects were HLA DRB1*0401.

### LV Transduction and Foxp3 Editing

CD4⁺ T cells were isolated from PBMC by magnetic bead CD4⁺ T cell isolation kit (Miltenyi) and cultured in RPMI 1640 media supplemented with 20% human serum and penicillin/streptomycin. T cells were activated with CD3/CD28 activator beads at a 1:1 bead to cell ratio and recombinant human IL-2, IL-7, and IL-15 at 50, 5, and 5 ng/ml, respectively on day 0. After 24 h activation, transduction with LV vectors encoding GAD65, IGRP, or PPI specific TCRs was performed by adding concentrated LV supernatant with polybrene at 10 μg/ml. Beads were removed after 24 h incubation and cells were either rested for 16-24 h for editing or expanded in media with IL-2 (20 ng/ml) until day 14 or day 15 to be used as Teff cells. For Foxp3 editing, cells were transfected by electroporation with RNP complex combined with Cas9 and guide RNA and then transduced with AAV template. 20-24 h after editing, cells were expanded in media with IL-2 (100 ng/ml) until day 10 and islet-specific LNGFR+ EngTregs were enriched by LNGFR magnetic beads. LNGFR− T cells also collected from the LNGFR⁺ cell enrichment to be used as controls in suppression assays.

In Vitro Expansion and Tetramer Staining of Islet-Specific T Cells Derived from PBMC

For expanding islet-specific T cells by peptide stimulation, CD4⁺ T cells (CD4+CD25−) were isolated from PBMC and incubated with irradiated autologous CD4⁻CD25⁺ cells and a pool of islet-specific peptides (GAD65₁₁₃₋₁₃₂, GAD65₂₆₅₋₂₈₄, GAD65₂₇₃₋₂₉₂, GAD65₃₀₅₋₃₂₄, GAD65₅₅₃₋₅₇₂, IGRP₁₇₋₃₆, IGRP₂₄₁₋₂₆₀, IGRP₃₀₅₋₃₂₄, and PPI₇₆₋₉₀) at 5 μg/ml. After 7 days of incubation, part of the T cells were harvested as day 7 islet-specific Teff and remaining cells were expanded in media with IL-2 at 20 ng/ml. IL-2 was added in 2-3 days of interval and cells were collected at day 14 as day 14 islet-specific Teff. In order to check population of expanded islet-specific T cells, day 14 Teff were incubated with PE-tagged tetramer for 1 h and followed by surface staining. For mechanistic experiments (Transwell suppression assay, IL-2 consumption, and TCR avidity), polyclonal islet-specific T cells were expanded with a pool of 9 islet-specific peptides (GAD65₁₁₃₋₁₃₂, GAD₂₆₅₋₂₈₄, GAD65₂₇₃₋₂₉₂, GAD65₃₀₅₋₃₂₄, GAD65₅₅₃₋₅₇₂, IGRP₁₇₋₃₆, IGRP₂₄₁₋₂₆₀, PPI₇₆₋₉₀, ZNT8₂₆₆₋₂₈₅) excluding IGRP₃₀₅₋₃₂₄ that is specific for T1D2 EngTregs to measure bystander suppression.

### In Vitro Suppression Assay Using CD3/CD28 Beads

2×10⁴ Teff were cultured alone or co-cultured with EngTregs or LNGFR⁻ T cells at 1:1 ratio in the presence of CD3/CD28 beads in 96 well plate. 1:28 or 1:32 of beads to Teff ratio was used for 3- or 4-day culture, respectively. Teff and EngTregs or LNGFR⁻ T cells were labeled with Cell Trace Violet (Invitrogen) and EF670 (Thermo Fisher), respectively, before the co-culture. Dilution of Cell Trace Violet (CTV) was measured as proliferation of Teff and % suppression was calculated as (a−b)/a×100 where a is the percentage Teff proliferation in the absence of Treg and b is the percentage of Teff proliferation in the presence of Treg.

### Antigen-Specific Suppression Assay

Autologous PBMC were irradiated at 5,000 rad and used as APC in the suppression assay using Teff transduced with TCR. CTV-labeled Teff were co-cultured with EF670-labeled EngTregs or islet-specific LNGFR⁻ T cells at 1:1 ratio in the presence of APC and DMSO or relevant peptide. Cells were incubated for 4 days and stained for measuring Teff proliferation. For measuring intracellular cytokines produced by Teff, cells were cultured for 3 days and incubated with Brefeldin A for another 4 h, followed by intracellular staining.

Antigen-Specific Suppression Assay Using Polyclonal Islet-Specific Teff Derived from T1D PBMC

CD14⁺ cells, CD4⁺CD25⁻, and CD4⁻CD25+ cells were isolated from 60 million PBMC of donors with T1D. CD14⁺ cells isolated using CD14 microbeads (Miltenyi) were cultured in media supplemented with GM-CSF and IL-4 at 800 U/ml and 1,000 U/ml, respectively, for 7 days to differentiate into monocyte-derived DC (mDC). CD4⁺CD25⁻ cells were divided, some used to generate EngTregs and the rest were used for in vitro expansion of polyclonal islet-specific Teff using 9 islet-specific peptides and irradiated autologous CD4⁻ CD25⁺ cells as described above. For the suppression assay, polyclonal islet-specific Teff harvested at day 7 or day 14 were co-cultured with or without poly EngTregs, T1D2 EngTregs, 4.13 EngTregs, or LNGFR− T cells in the presence of autologous mDC and DMSO or 9 islet-specific peptides for 4 days. EngTregs/LNGFR− T cells and polyclonal islet-specific Teff were labeled with EF670 and CTV, respectively, before the co-culture.

### Transwell Suppression Assay

mDC were pre-incubated with a pool of 10 islet-specific peptides (GAD65₁₁₃₋₁₃₂, GAD₂₆₅₋₂₈₄, GAD65₂₇₃₋₂₉₂, GAD65₃₀₅₋₃₂₄, GAD65₅₅₃₋₅₇₂, IGRP₁₇₋₃₆, IGRP₂₄₁₋₂₆₀, IGRP₃₀₅₋₃₂₄, PPI₇₆₋₉₀, ZNT8₂₆₆₋₂₈₅) for 1 hour, washed, and plated in both upper and lower chambers of 96 well transwell plate with pore size 0.4 μM (Corning). Polyclonal islet-specific Teff generated by stimulation with 9 islet-peptides (GAD65₁₁₃₋₁₃₂, GAD₂₆₅₋₂₈₄, GAD65₂₇₃₋₂₉₂, GAD65₃₀₅₋₃₂₄, GAD65₅₅₃₋₅₇₂, IGRP₁₇₋₃₆, IGRP₂₄₁₋₂₆₀, PPI₇₆₋₉₀, ZNT8₂₆₆₋₂₈₅) and T1D2 EngTregs were plated, where indicated. Cell populations being assessed for regulatory capacity were cultured in the upper chamber. Polyclonal islet Teff and T1D2 EngTregs were labeled with CTV and EF670, respectively, before the co-culture. After 4 days in culture, cells from both chambers were harvested and stained for FACS analysis. CTV dilution was measured to assess Teff proliferation.

### Suppression of APC Maturation Assay

CD14+ monocytes were isolated from PBMC and were cultured in the presence of GM-CSF and IL-4 for 7 days to differentiate into mDC. In the last 16-18 hours of culture, IFN-γ and CL075 were added for maturation. Matured mDC were co-cultured for 2 days with autologous CTV-labeled T1D2 EngTregs (or LNGFR− T cells) at 1:2 ratio of mDC to EngTregs/LNGFR−T cells in the presence of IGRP³⁰⁵⁻³²⁴ peptide. Cells were harvested and analyzed for surface marker expression (CD86 or CD80) on DC. MFI of CD86/CD80 on mDCs were normalized by MFI of mDC only condition. Data were normalized by dividing MFI of DC+ EngTregs or DC+LNGFR− by MFI of DC alone.

### Mice

NOD and NOD BDC2.5 mice were purchased from The Jackson Laboratory then bred and maintained at the Seattle Children's Research Institute (SCRI) SPF facility to produce the mice used in experiments here. Experimental NSG mice were purchased from The Jackson Laboratory, and acclimated at SCRI for 1-2 weeks before experiments. Experiments, breeding, and handling of mice were conducted in accordance with the National Institutes of Health's Guide for the Care and Use of Laboratory Animals using protocols approved by the Institutional Animal Care and Use Committee at the SCRI.

### Primary Mouse T Cell Isolation, Culture, Editing and Enrichment

To obtain CD4⁺ T cells for gene editing, mouse lymphocytes from spleen and lymph nodes of 8-12 weeks old NOD BDC2.5 and NOD mice were isolated and combined. CD4⁺ T cells were purified from lymphocytes by negative selection using EasySep mouse CD4⁺ T Cell Enrichment Kit (STEMCELL Technologies), then activated using mouse specific anti-CD3/CD28 coated beads (Gibco) for ˜40 hrs in a RPMI media containing 20% FBS (Omega Scientific Inc., Catalog #FB-11), HEPES, Glutamax, β-mercaptoethanol and 50 ng/mL mouse IL-2 (Peprotech). After activation, cells were separated from beads and further cultured for ˜10 hours in media then washed twice in PBS and resuspend in Buffer R (Neon kit, Invitrogen) at 25×10⁶ cells/mL. RNP was prepared in Buffer R by mixing 20 pmol of Cas9 (IDT) with 50 pmol of mouse Foxp3 specific gRNA for 25 min at room temperature. Delivery of RNP into mouse cells was achieved by electroporation (1550V, 10 ms and 3 pulses) using Neon system (Thermo Fisher Scientific) followed by incubation with AAV5 containing donor template with homology sequence to mouse Foxp3 for ˜20-24 hours at 37° C. Cells were replenished (two-fold dilution) with fresh media containing IL-2 and transferred to a new tissue culture dish for another ˜16 hours before final analysis and enrichment. Two days post editing, edited cells were collected, counted and stained for biotinylated anti-LNGFR antibody (Miltenyi Biotech) and enriched using anti-biotin microbeads (Miltenyi Biotech) as described (67).

Isolation and Enrichment of Antigen Specific Teff and tTreg

Murine effector CD4⁺ T cells used experimentally were CD4⁺ CD25⁻, and were enriched via negative selection of CD4 and CD25 (Miltenyi Biotec) from combined single cell suspensions obtained from spleen and lymph nodes of NOD BDC2.5 mice. Murine CD4⁺ Teff were freshly prepared for each experiment. CD4⁺ CD25⁺ tTreg from antigen-specific NOD BDC2.5 and polyclonal NOD mice were enriched using a murine Treg enrichment kit (Miltenyi Biotec) according to the manufacturer's instructions. Enriched (≥90%) tTreg were activated to match EngTregs activation status and timeline, in the same media used to culture EngTregs. Activated tTreg were immunophenotyped then cryopreserved in LN₂. Prior to injection, tTreg were thawed and rested in IL-2 containing media overnight. Viability and CD4⁺ CD25⁺ FOXP3⁺ phenotype was confirmed by flow cytometry prior to injection.

### Isolation of Diabetic NOD Splenocytes 659

Diabetic NOD mice were identified by weekly by urinalysis (AimStrip US-G; Germaine Laboratories), followed by confirmation of hyperglycemia using a Bayer Contour Blood Glucose Monitor System (Bayer). Mice that met diabetic criteria (>250 mg/dl) on two consecutive days were euthanized and splenocytes were isolated by manual dissociation, RBC lysis with ACK buffer followed by PBS washing and cryopreserved in serum-free medium (CryoStor CS10).

### Diabetes Induction and Monitoring

8-10 week-old male NSG mice were pre-screened for normal blood glucose values before enrolling in diabetes prevention studies. Mice were injected with 5×10⁴ islet specific Teff cells in combination with 5×10⁴ (1:1) mock-edited control, tTreg or EngTregs; in some conditions 1×10⁵ (1:2 Teff:Treg) Treg were injected. Cells were delivered via the retro-orbital sinus. For studies using NOD splenocytes to induce diabetes, mice were injected with 2.25×10⁶ splenocytes via the retro-orbital sinus, and 1×10⁵ EngTregs intravenously via tail vein. Diabetes was monitored by peripheral blood sampling using a Bayer Contour Blood Glucose Monitor System (Bayer). Mice with blood glucose >250 mg/dL twice within 24 hrs or exceeding 400 mg/dL were considered diabetic and were euthanized.

### Immunohistochemistry Studies

Immunohistochemistry for detection of insulin and CD3 on deparaffinized tissue sections was performed by the Histology and Imaging Core at the University of Washington using a Leica Bond Automated Immunostainer (Leica Microsystems). Rabbit polyclonal anti-insulin antibody (ab63820) was purchased from Abcam and used at a dilution of 1:1000 following an antigen retrieval step using citrate buffer. For detection of CD3, a rat monoclonal antibody (MCA1477) was purchased from AbD Serotec (Bio-Rad) and used at a dilution of 1:100 following an antigen retrieval with EDTA. All other reagents were provided by Leica specifically for use in the Leica Immunostainer. Pancreatic tissues were examined by a board-certified veterinary pathologist experienced in the evaluation of rodent tissues who was blinded to treatment groups. The total area of individual pancreas paraffin sections was measured using Nikon's NIS-Elements software (Nikon Microscopy). Total islet counts were performed using a 20× objective.

### Statistical Analysis

GraphPad Prism version 8 was used to conduct all statistical analyses. Specifics of the statistical tests used are indicated in each figure legend; no outliers were excluded.

### REFERENCES AND NOTES

1. M. Wallberg, et al. Trends in Immunology 34, 583-591 (2013).
2. A. Pugliese, et al. J Clin Invest 127, 2881-2891 (2017).
3. C. M. Hull, et al. Diabetologia 60, 1839-1850 (2017).
4. A. Ferraro, et al. Diabetes 60, 2903-2913 (2011).
5. S. Lindley, et al. Diabetes 54, 92-99 (2005).
6. J. A. Bluestone, et al. Sci Transl Med 7, 315ra189 (2015).
7. J. H. Esensten, et al. The Journal of allergy and clinical immunology 142, 1710-1718 (2018).
8. C. Raffin, et al. Nat Rev Immunol 20, 158-172 (2020).
9. Q. Tang, et al. The Journal of experimental medicine 199, 1455-1465 (2004).
10. K. V. Tarbell, et al. The Journal of experimental medicine 199, 1467-1477 (2004).
11. L. A. Stephens, et al. Eur J Immunol 39, 1108-1117 (2009).
12. P. Zhou, et al. J Immunol 172, 1515-1523 (2004).
13. K. Fujio, et al. J Immunol 177, 8140-8147 (2006).
14. G. P. Wright, et al. Proc Natl Acad Sci USA 106, 19078-19083 (2009).
15. J. Y. Tsang, et al. J Clin Invest 118, 3619-3628 (2008).
16. E. L. Masteller, et al. J Immunol 175, 3053-3059 (2005).
17. K. V. Tarbell, et al. The Journal of experimental medicine 204, 191-201 (2007).
18. C. G. Brunstein, et al. Blood 117, 1061-1070 (2011).
19. S. Sakaguchi, et al. Cell 133, 775-787 (2008).
20. K. L. Hippen, et al. Sci Transl Med 3, 83ra41 (2011).
21. C. Baecher-Allan, et al. J Immunol 167, 1245-1253 (2001).
22. J. A. Bluestone, et al. Science 362, 154-155 (2018).
23. Y. C. Kim, et al. J Autoimmun 92, 77-86 (2018).
24. Z. Zhang, et al. J Immunol 198, 2612-2625 (2017).
25. D. V. Sawant, et al. Immunol Rev 259, 173-191 (2014).
26. N. Komatsu, et al. Proc Natl Acad Sci USA 106, 1903-1908 (2009).
27. X. Zhou, et al. Nat Immunol 10, 1000-1007 (2009).
28. S. L. Bailey-Bucktrout, et al. Immunity 39, 949-962 (2013).
29. N. Komatsu, et al. Nat. Med. 20, 62-68 (2014).
30. Y. Honaker, et al. Sci Transl Med 12, (2020).
31. K. Cerosaletti, et al. J Immunol 199, 323-335 (2017).
32. B. Fehse, et al. Hum Gene Ther 8, 1815-1824 (1997).
33. B. D. Singer, et al. Frontiers in immunology 5, 46 (2014).
34. A. E. Herman, et al. The Journal of experimental medicine 199, 1479-1489 (2004).
35. K. Wing, et al. Science 322, 271-275 (2008).
36. D. K. Sojka, et al. J Immunol 175, 7274-7280 (2005).
37. N. Oberle, et al. J Immunol 179, 3578-3587 (2007).
38. A. Schmidt, et al. Science signaling 4, ra90 (2011).
39. A. Schmidt, et al. Frontiers in immunology 3, 51 (2012).
40. E. M. Shevach, et al. Frontiers in immunology 9, 1048 (2018).
41. N. A. Danke, et al. J Autoimmun. 25, 303-311 (2005).
42. J. Yang, et al. J Immunol. 176, 2781-2789 (2006).
43. J. Yang, et al. J. Autoimmun. 31, 30-41 (2008).
44. S. A. Long, et al. Eur. J Immunol. 39, 612-620 (2009).
45. J. Yang, et al., Immunology 138, 269-279 (2013).
46. D. A. et al. Nat. Rev. Immunol. 8, 523-532 (2008).
47. A. M. Thornton, et al. The Journal of experimental medicine 188, 287-296 (1998).
48. L. W. Collison, et al. J. Immunol. 182, 6121-6128 (2009).
49. Y. Onishi, et al. Proc Natl Acad Sci USA 105, 10113-10118 (2008).
50. N. A. J. Dawson, et al. Sci Transl Med 12, (2020).
51. P. Pandiyan, et al. Nat Immunol 8, 1353-1362 (2007).
52. D. Q. Tran, et al. J Immunol 182, 2929-2938 (2009).
53. J. D. Katz, et al. Cell 74, 1089-1100 (1993).
54. M. S. Anderson, et al. Annu Rev Immunol 23, 447-485 (2005).
55. K. Haskins, et al. Diabetes 45, 1299-1305 (1996).
56. K. Haskins, et al. Science 249, 1433-1436 (1990).
57. M. Presa, et al. J Immunol 195, 3011-3019 (2015).
58. Q. Tang, et al. Nat. Immunol. 9, 239-244 (2008).
59. J. L. McGovern, et al. Frontiers in immunology 8, 1517 (2017).
60. T. Maj, et al. Nat Immunol 18, 1332-1341 (2017).
61. L. Schmidleithner, et al. Immunity 50, 1232-1248.e1214 (2019).
62. J. Rana, et al. Molecular therapy: the journal of the American Society of Gene Therapy 29, 2660-2676 (2021).
63. J. Y. Tsang, et al. Am J Transplant 11, 1610-1620 (2011).
64. M. Bettini, et al. J Immunol 193, 571-579 (2014).
65. M. L. Sprouse, et al. J Immunol 200, 909-914 (2018).
66. M. S. Turner, et al. J Immunol 183, 4895-4903 (2009).
67. M. S. Turner, et al. Diabetologia 57, 1428-1436 (2014).
68. G. Plesa, et al. Blood 119, 3420-3430 (2012).
69. W. L. Yeh, et al. Frontiers in immunology 8, 1313 (2017).
70. S. Nair, et al. Curr Protoe Immunol Chapter 7, Unit7.32 (2012).
71. R. Gastpar, et al. J Immunol 172, 972-980 (2004).

All references cited are incorporated herein by reference in their entirety.

### Sequences

TABLE 1
Examples of amino acid sequences of TCR chains and portions thereof

| | | Region, | | |
|---|---|---|---|---|
| | | Domain, | | |
| | | or | | SEQ |
| | | Sequence | | ID |
| TCR | Antigen | Name | Amino Acid Sequence | NO: |
| T1D2 | IGRP | αCDR1 | NSMFDY | 1 |
| | (305-324) | αCDR2 | ISSIKDK | 2 |
| | | αCDR3 | CAATRTSGTYKYIF | 3 |
| | | βCDR1 | MNHNY | 4 |
| | | βCDR2 | SVGAGI | 5 |
| | | βCDR3 | CASSPWGAGGTDTQYF | 6 |
| | | Vα | MAMLLGASVLILWLQPDWVNSQQKNDDQQVKQNSPSLSVQE | 7 |
| | | | GRISILNCDYTNSMFDYFLWYKKYPAEGPTFLISISSIKDK | |
| | | | NEDGRFTVFLNKSAKHLSLHIVPSQPGDSAVYFCAATRTSG | |
| | | | TYKYIFGTGTRLKVLA | |
| | | Vβ | MSISLLCCAAFPLLWAGPVNAGVTQTPKFRILKIGQSMTLQ | 8 |
| | | | CTQDMNHNYMYWYRQDPGMGLKLIYYSVGAGITDKGEVPNG | |
| | | | YNVSRSTTEDFPLRLELAAPSQTSVYFCASS | |
| | | TCRα | MAMLLGASVLILWLQPDWVNSQQKNDDQQVKQNSPSLSVQE | 9 |
| | | (full- | GRISILNCDYTNSMFDYFLWYKKYPAEGPTFLISISSIKDK | |
| | | length) | NEDGRFTVFLNKSAKHLSLHIVPSQPGDSAVYFCAATRTSG | |
| | | | TYKYIFGTGTRLKVLANIQNPDPAVYQLRDSKSSDKSVCLF | |
| | | | TDFDSQTNVSQSKDSDVYITDKTVLDMRSMDFKSNSAVAWS | |
| | | | NKSDFACANAENNSIIPEDTFFPSPGKGSFGAFAGCFLASG | |
| | | | MARFCPELWSMMSKTPLIGGLG | |
| | | TCRß | MSISLLCCAAFPLLWAGPVNAGVTQTPKFRILKIGQSMTLQ | 10 |
| | | (full- | CTQDMNHNYMYWYRQDPGMGLKLIYYSVGAGITDKGEVPNG | |
| | | length) | YNVSRSTTEDFPLRLELAAPSQTSVYFCASSPWGAGGTDTQ | |
| | | | YFGPGTRLTVLEDLKNVEPPEVAVFEPSEAEISHTQKATLV | |
| | | | CLATGFYPDHVELSWWVNGKEVHSGVSTDPQPLKEQPALND | |
| | | | SRYCLSSRLRVSATFWQNPRNHFRCQVQFYGLSENDEWTQD | |
| | | | RAKPVTQIVSAEAWGRADCGFTSESYQQGVLSATILYEILL | |
| | | | GKATLYAVLVSALVLMAMVKRKDSRG | |
| T1D4 | IGRP | αCDR1 | VSNAYN | 11 |
| | (241-260) | αCDR2 | GSKP | 12 |
| | | αCDR3 | CAVEDLNQAGTALIF | 13 |
| | | βCDR1 | SGHRS | 14 |
| | | βCDR2 | YFSETQ | 15 |
| | | βCDR3 | CASSLALGQGNQQFF | 16 |
| | | Vα | MKSLRVLLVILWLQLSWVWSQKDQVFQPSTVASSEGAVVEI | 17 |
| | | | FCNHSVSNAYNFFWYLHFPGCAPRLLVKGSKPSQQGRYNMT | |
| | | | YERFSSSLLILQVREADAAVYYCAVEDLNQAGTALIFGKGT | |
| | | | TLSVSS | |
| | | Vβ | MGSRLLCWVLLCLLGAGPVKAGVTQTPRYLIKTRGQQVTLS | 18 |
| | | | CSPISGHRSVSWYQQTPGQGLQFLFEYFSETQRNKGNFPGR | |
| | | | FSGRQFSNSRSEMNVSTLELGDSALYLCASSLALGQGNQQF | |
| | | | FGPGTRLTVLEDLKNVFPPEVAVFEPSEAEISHTQKATLVC | |
| | | | LATGFYPDHVELSWWVNGKEVHSGVSTDPQPLKEQPALNDS | |
| | | | RYCLSSRLRVSATFWQNPRNHERCQVQFYGLSENDEWTQDR | |
| | | | AKPVTQIVSAEAWGRADCGFTSESYQQGVLSATILYEILLG | |
| | | | KATLYAVLVSALVLMAMVKRKDSRG | |
| | | TCRα | MKSLRVLLVILWLQLSWVWSQKDQVFQPSTVASSEGAVVEI | 19 |
| | | (full- | FCNHSVSNAYNFFWYLHFPGCAPRLLVKGSKPSQQGRYNMT | |
| | | length) | YERFSSSLLILQVREADAAVYYCAVEDLNQAGTALIFGKGT | |
| | | | TLSVSSNIQNPDPAVYQLRDSKSSDKSVCLFTDFDSQTNVS | |
| | | | QSKDSDVYITDKTVLDMRSMDFKSNSAVAWSNKSDFACANA | |
| | | | FNNSIIPEDTFFPSPGKGSFGAFAGCFLASGMARFCPELWS | |
| | | | MMSKTPLIGGLG | |
| | | TCRβ | MGSRLLCWVLLCLLGAGPVKAGVTQTPRYLIKTRGQQVTLS | 20 |
| | | (full- | CSPISGHRSVSWYQQTPGQGLQFLFEYFSETQRNKGNFPGR | |
| | | length) | FSGRQFSNSRSEMNVSTLELGDSALYLCASSLALGQGNQQF | |
| | | | FGPGTRLTVLEDLKNVEPPEVAVFEPSEAEISHTQKATLVC | |
| | | | LATGFYPDHVELSWWVNGKEVHSGVSTDPQPLKEQPALNDS | |
| | | | RYCLSSRLRVSATFWQNPRNHERCQVQFYGLSENDEWTQDR | |
| | | | AKPVTQIVSAEAWGRADCGFTSESYQQGVLSATILYEILLG | |
| | | | KATLYAVLVSALVLMAMVKRKDSRG | |
| T1D5- | IGRP | αCDR1 | TTLSN | 21 |
| 1 | (305-324) | αCDR2 | LVKSGEV | 22 |
| | | αCDR3 | CAGQTGANNLFF | 23 |
| | | βCDR1 | LGHNA | 24 |
| | | βCDR2 | YSLEER | 25 |
| | | βCDR3 | CASSQEVGTVPNQPQHF | 26 |
| | | Vα | MLLITSMLVLWMQLSQVNGQQVMQIPQYQHVQEGEDFTTYC | 27 |
| | | | NSSTTLSNIQWYKQRPGGHPVFLIQLVKSGEVKKQKRLTFQ | |
| | | | FGEAKKNSSLHITATQTTDVGTYFCAGQTGANNLFFGTGTR | |
| | | | LTVIP | |
| | | Vβ | MGCRLLCCAVLCLLGAVPMETGVTQTPRHLVMGMTNKKSLK | 28 |
| | | | CEQHLGHNAMYWYKQSAKKPLELMFVYSLEERVENNSVPSR | |
| | | | ESPECPNSSHLFLHLHTLQPEDSALYLCASSQEVGTVPNQP | |
| | | | QHFGDGTRLSIL | |
| | | TCRα | MLLITSMLVLWMQLSQVNGQQVMQIPQYQHVQEGEDFTTYC | 29 |
| | | (full- | NSSTTLSNIQWYKQRPGGHPVFLIQLVKSGEVKKQKRLTFQ | |
| | | length) | FGEAKKNSSLHITATQTTDVGTYFCAGQTGANNLFFGTGTR | |
| | | | LTVIPYIQNPDPAVYQLRDSKSSDKSVCLFTDFDSQTNVSQ | |
| | | | SKDSDVYITDKTVLDMRSMDFKSNSAVAWSNKSDFACANAF | |
| | | | NNSIIPEDTFFPSPGKGSFGAFAGCFLASGMARFCPELWSM | |
| | | | MSKTPLIGGLG | |
| | | TCRβ | MGCRLLCCAVLCLLGAVPMETGVTQTPRHLVMGMTNKKSLK | 30 |
| | | (full- | CEQHLGHNAMYWYKQSAKKPLELMFVYSLEERVENNSVPSR | |
| | | length) | FSPECPNSSHLFLHLHTLQPEDSALYLCASSQEVGTVPNQP | |
| | | | QHFGDGTRLSILEDLNKVFPPEVAVFEPSEAEISHTQKATL | |
| | | | VCLATGFFPDHVELSWWVNGKEVHSGVSTDPQPLKEQPALN | |
| | | | DSRYCLSSRLRVSATFWQNPRNHERCQVQFYGLSENDEWTQ | |
| | | | DRAKPVTQIVSAEAWGRADCGFTSVSYQQGVLSATILYEIL | |
| | | | LGKATLYAVLVSALVLMAMVKRKDF | |

TABLE 2
Examples of nucleic acid sequences encoding TCR chains and portions thereof

| | | Region, | | |
|---|---|---|---|---|
| | | Domain, | | |
| | | or | | SEQ |
| | | Sequence | | ID |
| TCR | Antigen | Name | Nucleotide Sequence | NO: |
| T1D2 | | αCDR1 | AACAGCATGTTTGATTAT | 31 |
| | | αCDR2 | ATAAGTTCCATTAAGGATAAA | 32 |
| | | αCDR3 | TGTGCAGCAACCCGTACCTCAGGAACCTACAAATACATCT | 33 |
| | | | TT | |
| | | βCDR1 | ATGAACCATAACTAC | 34 |
| | | βCDR2 | TCAGTTGGTGCTGGTATC | 35 |
| | | βCDR3 | TGTGCCAGCAGTCCGTGGGGGGCAGGCGGCACAGATACGC | 36 |
| | | | AGTATTTT | |
| | | Vα | ATGGCCATGCTCCTGGGGGCATCAGTGCTGATTCTGTGGC | 37 |
| | | | TTCAGCCAGACTGGGTAAACAGTCAACAGAAGAATGATGA | |
| | | | CCAGCAAGTTAAGCAAAATTCACCATCCCTGAGCGTCCAG | |
| | | | GAAGGAAGAATTTCTATTCTGAACTGTGACTATACTAACA | |
| | | | GCATGTTTGATTATTTCCTATGGTACAAAAAATACCCTGC | |
| | | | TGAAGGTCCTACATTCCTGATATCTATAAGTTCCATTAAG | |
| | | | GATAAAAATGAAGATGGAAGATTCACTGTCTTCTTAAACA | |
| | | | AAAGTGCCAAGCACCTCTCTCTGCACATTGTGCCCTCCCA | |
| | | | GCCTGGAGACTCTGCAGTGTACTTCTGTGCAGCAACCCGT | |
| | | | ACCTCAGGAACCTACAAATACATCTTTGGAACAGGCACCA | |
| | | | GGCTGAAGGTTTTAGCAA | |
| | | Vβ | ATGAGCATCAGCCTCCTGTGCTGTGCAGCCTTTCCTCTCC | 38 |
| | | | TGTGGGCAGGTCCAGTGAATGCTGGTGTCACTCAGACCCC | |
| | | | AAAATTCCGCATCCTGAAGATAGGACAGAGCATGACACTG | |
| | | | CAGTGTACCCAGGATATGAACCATAACTACATGTACTGGT | |
| | | | ATCGACAAGACCCAGGCATGGGGCTGAAGCTGATTTATTA | |
| | | | TTCAGTTGGTGCTGGTATCACTGATAAAGGAGAAGTCCCG | |
| | | | AATGGCTACAACGTCTCCAGATCAACCACAGAGGATTTCC | |
| | | | CGCTCAGGCTGGAGTTGGCTGCTCCCTCCCAGACATCTGT | |
| | | | GTACTTCTGTGCCAGCAGTCCGTGGGGGGCAGGCGGCACA | |
| | | | GATACGCAGTATTTTGGCCCAGGCACCCGGCTGACAGTGC | |
| | | | TC | |
| | | TCRα | ATGGCCATGCTCCTGGGGGCATCAGTGCTGATTCTGTGGC | 39 |
| | | (full- | TTCAGCCAGACTGGGTAAACAGTCAACAGAAGAATGATGA | |
| | | length) | CCAGCAAGTTAAGCAAAATTCACCATCCCTGAGCGTCCAG | |
| | | | GAAGGAAGAATTTCTATTCTGAACTGTGACTATACTAACA | |
| | | | GCATGTTTGATTATTTCCTATGGTACAAAAAATACCCTGC | |
| | | | TGAAGGTCCTACATTCCTGATATCTATAAGTTCCATTAAG | |
| | | | GATAAAAATGAAGATGGAAGATTCACTGTCTTCTTAAACA | |
| | | | AAAGTGCCAAGCACCTCTCTCTGCACATTGTGCCCTCCCA | |
| | | | GCCTGGAGACTCTGCAGTGTACTTCTGTGCAGCAACCCGT | |
| | | | ACCTCAGGAACCTACAAATACATCTTTGGAACAGGCACCA | |
| | | | GGCTGAAGGTTTTAGCAAATATCCAGAACCCTGACCCTGC | |
| | | | AGTATATCAGCTGAGAGACTCTAAATCCAGTGACAAGTCT | |
| | | | GTCTGCCTATTCACCGATTTTGATTCTCAAACAAATGTGT | |
| | | | CACAAAGTAAGGATTCTGATGTGTATATCACAGACAAAAC | |
| | | | TGTGCTAGACATGAGGTCTATGGACTTCAAGAGCAACAGT | |
| | | | GCTGTGGCCTGGAGCAACAAATCTGACTTTGCATGTGCAA | |
| | | | ACGCCTTCAACAACAGCATTATTCCAGAAGACACCTTCTT | |
| | | | CCCCAGCCCAGGTAAGGGCAGCTTTGGTGCCTTCGCAGGC | |
| | | | TGTTTCCTTGCTTCAGGAATGGCCAGGTTCTGCCCAGAGC | |
| | | | TCTGGTCAATGATGTCTAAAACTCCTCTGATTGGTGGTCT | |
| | | | CGGC | |
| | | TCRβ | ATGAGCATCAGCCTCCTGTGCTGTGCAGCCTTTCCTCTCC | 40 |
| | | (full- | TGTGGGCAGGTCCAGTGAATGCTGGTGTCACTCAGACCCC | |
| | | length) | AAAATTCCGCATCCTGAAGATAGGACAGAGCATGACACTG | |
| | | | CAGTGTACCCAGGATATGAACCATAACTACATGTACTGGT | |
| | | | ATCGACAAGACCCAGGCATGGGGCTGAAGCTGATTTATTA | |
| | | | TTCAGTTGGTGCTGGTATCACTGATAAAGGAGAAGTCCCG | |
| | | | AATGGCTACAACGTCTCCAGATCAACCACAGAGGATTTCC | |
| | | | CGCTCAGGCTGGAGTTGGCTGCTCCCTCCCAGACATCTGT | |
| | | | GTACTTCTGTGCCAGCAGTCCGTGGGGGGCAGGCGGCACA | |
| | | | GATACGCAGTATTTTGGCCCAGGCACCCGGCTGACAGTGC | |
| | | | TCGAGGACCTTAAGAATGTGTTCCCTCCCGAGGTGGCTGT | |
| | | | CTTCGAACCAAGCGAAGCCGAGATCTCTCACACACAAAAG | |
| | | | GCTACTCTCGTGTGTCTGGCAACGGGTTTTTACCCTGATC | |
| | | | ATGTAGAGTTGTCTTGGTGGGTTAACGGCAAAGAAGTACA | |
| | | | CTCCGGTGTAAGCACCGATCCTCAGCCACTTAAGGAACAA | |
| | | | CCCGCACTCAACGACTCCAGATACTGTTTGAGTTCTAGGC | |
| | | | TGAGAGTCTCAGCGACGTTTTGGCAGAACCCACGGAATCA | |
| | | | TTTCAGATGTCAGGTCCAGTTTTACGGGTTGAGCGAGAAC | |
| | | | GACGAGTGGACACAGGATCGGGCTAAACCAGTGACCCAGA | |
| | | | TTGTATCAGCCGAAGCATGGGGAAGGGCAGATTGCGGCTT | |
| | | | TACCTCAGAGTCATATCAACAGGGCGTTTTGTCTGCAACA | |
| | | | ATCCTTTATGAGATCCTGCTTGGTAAGGCGACTCTGTATG | |
| | | | CCGTGCTTGTTAGTGCCCTGGTTCTGATGGCAATGGTAAA | |
| | | | AAGAAAGGACTCTAGGGGA | |
| T1D4 | | αCDR1 | GTGTCCAACGCCTACAAT | 41 |
| | | αCDR2 | GGCTCTAAGCCC | 42 |
| | | αCDR3 | TGTGCCGTGGAGGATCTGAATCAGGCCGGCACAGCCCTGA | 43 |
| | | | TTTTC | |
| | | βCDR1 | TCCGGACACCGCTCC | 44 |
| | | βCDR2 | TATTTCTCTGAGACACAG | 45 |
| | | βCDR3 | TGCGCAAGCTCCCTGGCCCTGGGACAGGGGAATCAGCAGT | 46 |
| | | | TTTTC | |
| | | Vα | ATGAAGAGCCTGCGCGTGCTGCTGGTCATCCTGTGGCTGC | 47 |
| | | | AATTGAGTTGGGTGTGGAGCCAGAAGGACCAGGTGTTTCA | |
| | | | GCCTAGCACTGTCGCATCATCAGAGGGGGCCGTCGTGGAA | |
| | | | ATCTTTTGTAATCACAGCGTGTCCAACGCCTACAATTTCT | |
| | | | TTTGGTATCTGCACTTCCCAGGATGCGCACCTAGGCTGCT | |
| | | | GGTGAAGGGCTCTAAGCCCAGCCAGCAGGGCCGGTACAAC | |
| | | | ATGACCTATGAGCGGTTCAGCTCCTCTCTGCTGATCCTGC | |
| | | | AGGTGCGGGAGGCAGACGCAGCCGTGTACTATTGTGCCGT | |
| | | | GGAGGA | |
| | | Vβ | ATGGGATCAAGACTGCTGTGCTGGGTGCTGCTGTGCCTGC | 48 |
| | | | TGGGAGCCGGACCTGTGAAAGCCGGGGTGACTCAGACTCC | |
| | | | ACGATACCTGATCAAGACCAGGGGCCAGCAGGTGACACTG | |
| | | | TCTTGCAGCCCAATCTCCGGACACCGCTCCGTGTCTTGGT | |
| | | | ACCAGCAGACCCCTGGACAGGGACTGCAGTTCCTGTTTGA | |
| | | | GTATTTCTCTGAGACACAGCGGAACAAGGGCAATTTCCCC | |
| | | | GGCCGGTTTAGCGGCAGACAGTTTAGCAACTCCAGGTCTG | |
| | | | AGATGAATGTGAGCACCCTGGAGCTGGGCGACTCCGCCCT | |
| | | | GTACCTGTGCGCAAGCTCCCTGGCCCTGGGACAGGGGAAT | |
| | | | CAGCAGTTTTTCGGACCCGGAACAAGGCTGACCGTGCTGG | |
| | | TCRα | ATGAAGAGCCTGCGCGTGCTGCTGGTCATCCTGTGGCTGC | 49 |
| | | (full- | AATTGAGTTGGGTGTGGAGCCAGAAGGACCAGGTGTTTCA | |
| | | length) | GCCTAGCACTGTCGCATCATCAGAGGGGGCCGTCGTGGAA | |
| | | | ATCTTTTGTAATCACAGCGTGTCCAACGCCTACAATTTCT | |
| | | | TTTGGTATCTGCACTTCCCAGGATGCGCACCTAGGCTGCT | |
| | | | GGTGAAGGGCTCTAAGCCCAGCCAGCAGGGCCGGTACAAC | |
| | | | ATGACCTATGAGCGGTTCAGCTCCTCTCTGCTGATCCTGC | |
| | | | AGGTGCGGGAGGCAGACGCAGCCGTGTACTATTGTGCCGT | |
| | | | GGAGGATCTGAATCAGGCCGGCACAGCCCTGATTTTCGGC | |
| | | | AAAGGAACAACCCTGAGCGTGAGCAGCAATATCCAGAACC | |
| | | | CTGACCCTGCAGTATATCAGCTGAGAGACTCTAAATCCAG | |
| | | | TGACAAGTCTGTCTGCCTATTCACCGATTTTGATTCTCAA | |
| | | | ACAAATGTGTCACAAAGTAAGGATTCTGATGTGTATATCA | |
| | | | CAGACAAAACTGTGCTAGACATGAGGTCTATGGACTTCAA | |
| | | | GAGCAACAGTGCTGTGGCCTGGAGCAACAAATCTGACTTT | |
| | | | GCATGTGCAAACGCCTTCAACAACAGCATTATTCCAGAAG | |
| | | | ACACCTTCTTCCCCAGCCCAGGTAAGGGCAGCTTTGGTGC | |
| | | | CTTCGCAGGCTGTTTCCTTGCTTCAGGAATGGCCAGGTTC | |
| | | | TGCCCAGAGCTCTGGTCAATGATGTCTAAAACTCCTCTGA | |
| | | | TTGGTGGTCTCGGC | |
| | | TCRβ | ATGGGATCAAGACTGCTGTGCTGGGTGCTGCTGTGCCTGC | 50 |
| | | (full- | TGGGAGCCGGACCTGTGAAAGCCGGGGTGACTCAGACTCC | |
| | | length) | ACGATACCTGATCAAGACCAGGGGCCAGCAGGTGACACTG | |
| | | | TCTTGCAGCCCAATCTCCGGACACCGCTCCGTGTCTTGGT | |
| | | | ACCAGCAGACCCCTGGACAGGGACTGCAGTTCCTGTTTGA | |
| | | | GTATTTCTCTGAGACACAGCGGAACAAGGGCAATTTCCCC | |
| | | | GGCCGGTTTAGCGGCAGACAGTTTAGCAACTCCAGGTCTG | |
| | | | AGATGAATGTGAGCACCCTGGAGCTGGGCGACTCCGCCCT | |
| | | | GTACCTGTGCGCAAGCTCCCTGGCCCTGGGACAGGGGAAT | |
| | | | CAGCAGTTTTTCGGACCCGGAACAAGGCTGACCGTGCTGG | |
| | | | AGGACCTTAAGAATGTGTTCCCTCCCGAGGTGGCTGTCTT | |
| | | | CGAACCAAGCGAAGCCGAGATCTCTCACACACAAAAGGCT | |
| | | | ACTCTCGTGTGTCTGGCAACGGGTTTTTACCCTGATCATG | |
| | | | TAGAGTTGTCTTGGTGGGTTAACGGCAAAGAAGTACACTC | |
| | | | CGGTGTATCTACCGATCCTCAGCCACTTAAGGAACAACCC | |
| | | | GCACTCAACGACTCCAGATACTGTTTGAGTTCTAGGCTGA | |
| | | | GAGTCTCAGCGACGTTTTGGCAGAACCCACGGAATCATTT | |
| | | | CAGATGTCAGGTCCAGTTTTACGGGTTGAGCGAGAACGAC | |
| | | | GAGTGGACACAGGATCGGGCTAAACCAGTGACCCAGATTG | |
| | | | TATCAGCCGAAGCATGGGGAAGGGCAGATTGCGGCTTTAC | |
| | | | CTCAGAGTCATATCAACAGGGCGTTTTGTCTGCAACAATC | |
| | | | CTTTATGAGATCCTGCTTGGTAAGGCGACTCTGTATGCCG | |
| | | | TGCTTGTTAGTGCCCTGGTTCTGATGGCAATGGTAAAAAG | |
| | | | AAAGGACTCTAGGGGA | |
| T1D5- | | αCDR1 | ACTACTTTAAGCAAT | 51 |
| 1 | | αCDR2 | TTAGTGAAGAGTGGAGAAGTG | 52 |
| | | αCDR3 | TGTGCAGGTCAAACTGGGGCAAACAACCTCTTCTTT | 53 |
| | | βCDR1 | CTGGGTCATAACGCT | 54 |
| | | βCDR2 | TACAGTCTTGAAGAACGG | 55 |
| | | βCDR3 | TGCGCCAGCAGCCAAGAAGTAGGTACAGTCCCCAATCAGC | 56 |
| | | | CCCAGCATTTT | |
| | | Vα | ATGCTACTCATCACATCAATGTTGGTCTTATGGATGCAAT | 57 |
| | | | TGTCACAGGTGAATGGACAACAGGTAATGCAAATTCCTCA | |
| | | | GTACCAGCATGTACAAGAAGGAGAGGACTTCACCACGTAC | |
| | | | TGCAATTCCTCAACTACTTTAAGCAATATACAGTGGTATA | |
| | | | AGCAAAGGCCTGGTGGACATCCCGTTTTTTTGATACAGTT | |
| | | | AGTGAAGAGTGGAGAAGTGAAGAAGCAGAAAAGACTGACA | |
| | | | TTTCAGTTTGGAGAAGCAAAAAAGAACAGCTCCCTGCACA | |
| | | | TCACAGCCACCCAGACTACAGATGTAGGAACCTACTTCTG | |
| | | | TGCAGGTCAAACTGGGGCAAACAACCTCTTCTTTGGGACT | |
| | | | GGAACGAGACTCACCGTTATTCCCT | |
| | | Vβ | ATGGGCTGCAGGCTGCTCTGCTGTGCGGTTCTCTGTCTCC | 58 |
| | | | TGGGAGCGGTCCCCATGGAAACGGGAGTTACGCAGACACC | |
| | | | AAGACACCTGGTCATGGGAATGACAAATAAGAAGTCTTTG | |
| | | | AAATGTGAACAACATCTGGGTCATAACGCTATGTATTGGT | |
| | | | ACAAGCAAAGTGCTAAGAAGCCACTGGAGCTCATGTTTGT | |
| | | | CTACAGTCTTGAAGAACGGGTTGAAAACAACAGTGTGCCA | |
| | | | AGTCGCTTCTCACCTGAATGCCCCAACAGCTCTCACTTAT | |
| | | | TCCTTCACCTACACACCCTGCAGCCAGAAGACTCGGCCCT | |
| | | | GTATCTCTGCGCCAGCAGCCAAGAAGTAGGTACAGTCCCC | |
| | | | AATCAGCCCCAGCATTTTGGTGATGGGACTCGACTCTCCA | |
| | | | TCCTAG | |
| | | TCRα | ATGCTACTCATCACATCAATGTTGGTCTTATGGATGCAAT | 59 |
| | | (full- | TGTCACAGGTGAATGGACAACAGGTAATGCAAATTCCTCA | |
| | | length) | GTACCAGCATGTACAAGAAGGAGAGGACTTCACCACGTAC | |
| | | | TGCAATTCCTCAACTACTTTAAGCAATATACAGTGGTATA | |
| | | | AGCAAAGGCCTGGTGGACATCCCGTTTTTTTGATACAGTT | |
| | | | AGTGAAGAGTGGAGAAGTGAAGAAGCAGAAAAGACTGACA | |
| | | | TTTCAGTTTGGAGAAGCAAAAAAGAACAGCTCCCTGCACA | |
| | | | TCACAGCCACCCAGACTACAGATGTAGGAACCTACTTCTG | |
| | | | TGCAGGTCAAACTGGGGCAAACAACCTCTTCTTTGGGACT | |
| | | | GGAACGAGACTCACCGTTATTCCCTATATCCAGAACCCTG | |
| | | | ACCCTGCAGTATATCAGCTGAGAGACTCTAAATCCAGTGA | |
| | | | CAAGTCTGTCTGCCTATTCACCGATTTTGATTCTCAAACA | |
| | | | AATGTGTCACAAAGTAAGGATTCTGATGTGTATATCACAG | |
| | | | ACAAAACTGTGCTAGACATGAGGTCTATGGACTTCAAGAG | |
| | | | CAACAGTGCTGTGGCCTGGAGCAACAAATCTGACTTTGCA | |
| | | | TGTGCAAACGCCTTCAACAACAGCATTATTCCAGAAGACA | |
| | | | CCTTCTTCCCCAGCCCAGGTAAGGGCAGCTTTGGTGCCTT | |
| | | | CGCAGGCTGTTTCCTTGCTTCAGGAATGGCCAGGTTCTGC | |
| | | | CCAGAGCTCTGGTCAATGATGTCTAAAACTCCTCTGATTG | |
| | | | GTGGTCTCGGC | |
| | | TCRβ | ATGGGCTGCAGGCTGCTCTGCTGTGCGGTTCTCTGTCTCC | 60 |
| | | (full- | TGGGAGCGGTCCCCATGGAAACGGGAGTTACGCAGACACC | |
| | | length) | AAGACACCTGGTCATGGGAATGACAAATAAGAAGTCTTTG | |
| | | | AAATGTGAACAACATCTGGGTCATAACGCTATGTATTGGT | |
| | | | ACAAGCAAAGTGCTAAGAAGCCACTGGAGCTCATGTTTGT | |
| | | | CTACAGTCTTGAAGAACGGGTTGAAAACAACAGTGTGCCA | |
| | | | AGTCGCTTCTCACCTGAATGCCCCAACAGCTCTCACTTAT | |
| | | | TCCTTCACCTACACACCCTGCAGCCAGAAGACTCGGCCCT | |
| | | | GTATCTCTGCGCCAGCAGCCAAGAAGTAGGTACAGTCCCC | |
| | | | AATCAGCCCCAGCATTTTGGTGATGGGACTCGACTCTCCA | |
| | | | TCCTAGAGGACCTTAATAAGGTGTTCCCTCCCGAGGTGGC | |
| | | | TGTCTTCGAACCAAGCGAAGCCGAGATCTCTCACACACAA | |
| | | | AAGGCTACTCTCGTGTGTCTGGCAACGGGTTTTTTCCCTG | |
| | | | ATCATGTAGAGTTGTCTTGGTGGGTTAACGGCAAAGAAGT | |
| | | | ACACTCCGGTGTAAGCACCGATCCTCAGCCACTTAAGGAA | |
| | | | CAACCCGCACTCAACGACTCCAGATACTGTTTGAGTTCTA | |
| | | | GGCTGAGAGTCTCAGCGACGTTTTGGCAGAACCCACGGAA | |
| | | | TCATTTCAGATGTCAGGTCCAGTTTTACGGGTTGAGCGAG | |
| | | | AACGACGAGTGGACACAGGATCGGGCTAAACCAGTGACCC | |
| | | | AGATTGTATCAGCCGAAGCATGGGGAAGGGCAGATTGCGG | |
| | | | CTTTACCTCAGTTTCATATCAACAGGGCGTTTTGTCTGCA | |
| | | | ACAATCCTTTATGAGATCCTGCTTGGTAAGGCGACTCTGT | |
| | | | ATGCCGTGCTTGTTAGTGCCCTGGTTCTGATGGCAATGGT | |
| | | | AAAAAGAAAGGACTTT | |

TABLE 3
Examples of amino acid sequences of CISC components and portions thereof

| | | | SEQ |
|---|---|---|---|
| CISC | Domain or | | ID |
| Component | Sequence Name | Amino Acid Sequence | NO: |
| Signal | LCN2 | MPLGLLWLGLALLGALHAQA | 61 |
| Peptide | | | |
| FKBP-IL2Rg | Extracellular | GVQVETISPGDGRTFPKRGQTCVVHYTGMLEDGKKFDS | 62 |
| | binding | SRDRNKPFKFMLGKQEVIRGWEEGVAQMSVGQRAKLTI | |
| | (FKBP) domain | SPDYAYGATGHPGIIPPHATLVEDVELLKLGE | |
| | IL-2Rg | GSNTSKENPFLFALEA | 63 |
| | extracellular | | |
| | domain | | |
| | fragment | | |
| | IL-2Rg | VVISVGSMGLIISLLCVYFWL | 64 |
| | transmembrane | | |
| | domain | | |
| | IL-2Rg | ERTMPRIPTLKNLEDLVTEYHGNFSAWSGVSKGLAESL | 65 |
| | cytoplasmic | QPDYSERLCLVSEIPPKGGALGEGPGASPCNQHSPYWA | |
| | domain | PPCYTLKPET | |
| | FKBP-IL2Rg | GVQVETISPGDGRTFPKRGQTCVVHYTGMLEDGKKFDS | 66 |
| | (full-length) | SRDRNKPFKFMLGKQEVIRGWEEGVAQMSVGQRAKLTI | |
| | | SPDYAYGATGHPGIIPPHATLVFDVELLKLGEGSNTSK | |
| | | ENPFLFALEAVVISVGSMGLIISLLCVYFWLERTMPRI | |
| | | PTLKNLEDLVTEYHGNFSAWSGVSKGLAESLQPDYSER | |
| | | LCLVSEIPPKGGALGEGPGASPCNQHSPYWAPPCYTLK | |
| | | PET | |
| FRB-IL2Rb | Extracellular | EMWHEGLEEASRLYFGERNVKGMFEVLEPLHAMMERGP | 67 |
| | binding (FRB) | QTLKETSFNQAYGRDLMEAQEWCRKYMKSGNVKDLTQA | |
| | domain | WDLYYHVERRISK | |
| | IL-2Rb | GKDT | 68 |
| | extracellular | | |
| | domain | | |
| | fragment | | |
| | IL-2Rb | IPWLGHLLVGLSGAFGFIILVYLLI | 69 |
| | transmembrane | | |
| | domain | | |
| | IL-2Rb | NCRNTGPWLKKVLKCNTPDPSKFFSQLSSEHGGDVQKW | 70 |
| | cytoplasmic | LSSPFPSSSFSPGGLAPEISPLEVLERDKVTQLLLQQD | |
| | domain | KVPEPASLSSNHSLTSCFTNQGYFFFHLPDALEIEACQ | |
| | | VYFTYDPYSEEDPDEGVAGAPTGSSPQPLQPLSGEDDA | |
| | | YCTFPSRDDLLLFSPSLLGGPSPPSTAPGGSGAGEERM | |
| | | PPSLQERVPRDWDPQPLGPPTPGVPDLVDFQPPPELVL | |
| | | REAGEEVPDAGPREGVSFPWSRPPGQGEFRALNARLPL | |
| | | NTDAYLSLQELQGQDPTHLV | |
| | FRB-IL2Rb | EMWHEGLEEASRLYFGERNVKGMFEVLEPLHAMMERGP | 71 |
| | (full-length) | QTLKETSFNQAYGRDLMEAQEWCRKYMKSGNVKDLTQA | |
| | | WDLYYHVERRISKGKDTIPWLGHLLVGLSGAFGFIILV | |
| | | YLLINCRNTGPWLKKVLKCNTPDPSKFFSQLSSEHGGD | |
| | | VQKWLSSPFPSSSESPGGLAPEISPLEVLERDKVTQLL | |
| | | LQQDKVPEPASLSSNHSLTSCFTNQGYFFFHLPDALEI | |
| | | EACQVYFTYDPYSEEDPDEGVAGAPTGSSPQPLQPLSG | |
| | | EDDAYCTFPSRDDLLLESPSLLGGPSPPSTAPGGSGAG | |
| | | EERMPPSLQERVPRDWDPQPLGPPTPGVPDLVDFQPPP | |
| | | ELVLREAGEEVPDAGPREGVSFPWSRPPGQGEFRALNA | |
| | | RLPLNTDAYLSLQELQGQDPTHLV | |
| Cytosolic | Cytosolic FRB | EMWHEGLEEASRLYFGERNVKGMFEVLEPLHAMMERGP | 72 |
| FRB domain | domain | QTLKETSFNQAYGRDLMEAQEWCRKYMKSGNVKDLTQA | |
| | | WDLYYHVERRISK | |

TABLE 4
Examples of nucleic acid sequences encoding CISC components and portions
thereof

| | | | SEQ |
|---|---|---|---|
| CISC | Domain or | | ID |
| Component | Sequence Name | Nucleotide Sequence | NO: |
| Signal | LCN2 | ATGCCTCTGGGCCTGCTGTGGCTGGGCCTGGCCCTGCT | 73 |
| Peptide | | GGGCGCCCTGCACGCCCAGGCC | |
| FKBP-IL2Rg | Extracellular | GGCGTGCAGGTGGAGACAATCTCCCCAGGCGACGGACG | 74 |
| | binding | CACATTCCCTAAGCGGGGCCAGACCTGCGTGGTGCACT | |
| | (FKBP) domain | ATACAGGCATGCTGGAGGATGGCAAGAAGTTTGACAGC | |
| | | TCCCGGGATAGAAACAAGCCATTCAAGTTTATGCTGGG | |
| | | CAAGCAGGAAGTGATCAGAGGCTGGGAGGAGGGCGTGG | |
| | | CCCAGATGTCTGTGGGCCAGAGGGCCAAGCTGACCATC | |
| | | AGCCCAGACTACGCCTATGGAGCAACAGGCCACCCAGG | |
| | | AATCATCCCACCTCACGCCACCCTGGTGTTCGATGTGG | |
| | | AGCTGCTGAAGCTGGGCGAG | |
| | IL-2Rg | GGATCCAACACATCAAAAGAGAACCCCTTTCTGTTCGC | 75 |
| | extracellular | ATTGGAGGCC | |
| | domain | | |
| | fragment | | |
| | IL-2Rg | GTAGTCATATCTGTTGGATCCATGGGACTTATTATCTC | 76 |
| | transmembrane | CCTGTTGTGTGTGTACTTCTGGCTG | |
| | domain | | |
| | IL-2Rg | GAACGGACTATGCCCAGGATCCCCACGCTCAAGAATCT | 77 |
| | cytoplasmic | GGAAGATCTCGTCACAGAATACCATGGTAATTTCAGCG | |
| | domain | CCTGGAGCGGAGTCTCTAAGGGTCTGGCCGAATCCCTC | |
| | | CAACCCGATTATTCTGAACGGTTGTGCCTCGTATCCGA | |
| | | AATACCACCAAAAGGCGGGGCTCTGGGTGAGGGCCCAG | |
| | | GGGCGAGTCCGTGCAATCAACACAGCCCGTATTGGGCC | |
| | | CCTCCTTGTTATACGTTGAAGCCCGAAACT | |
| | FKBP-IL2Rg | GGCGTGCAGGTGGAGACAATCTCCCCAGGCGACGGACG | 78 |
| | (full-length) | CACATTCCCTAAGCGGGGCCAGACCTGCGTGGTGCACT | |
| | | ATACAGGCATGCTGGAGGATGGCAAGAAGTTTGACAGC | |
| | | TCCCGGGATAGAAACAAGCCATTCAAGTTTATGCTGGG | |
| | | CAAGCAGGAAGTGATCAGAGGCTGGGAGGAGGGCGTGG | |
| | | CCCAGATGTCTGTGGGCCAGAGGGCCAAGCTGACCATC | |
| | | AGCCCAGACTACGCCTATGGAGCAACAGGCCACCCAGG | |
| | | AATCATCCCACCTCACGCCACCCTGGTGTTCGATGTGG | |
| | | AGCTGCTGAAGCTGGGCGAGGGATCCAACACATCAAAA | |
| | | GAGAACCCCTTTCTGTTCGCATTGGAGGCCGTAGTCAT | |
| | | ATCTGTTGGATCCATGGGACTTATTATCTCCCTGTTGT | |
| | | GTGTGTACTTCTGGCTGGAACGGACTATGCCCAGGATC | |
| | | CCCACGCTCAAGAATCTGGAAGATCTCGTCACAGAATA | |
| | | CCATGGTAATTTCAGCGCCTGGAGCGGAGTCTCTAAGG | |
| | | GTCTGGCCGAATCCCTCCAACCCGATTATTCTGAACGG | |
| | | TTGTGCCTCGTATCCGAAATACCACCAAAAGGCGGGGC | |
| | | TCTGGGTGAGGGCCCAGGGGCGAGTCCGTGCAATCAAC | |
| | | ACAGCCCGTATTGGGCCCCTCCTTGTTATACGTTGAAG | |
| | | CCCGAAACT | |
| FRB-IL2Rb | Extracellular | GAGATGTGGCACGAGGGCCTGGAGGAGGCCAGCAGGCT | 79 |
| | binding (FRB) | GTATTTTGGCGAGCGCAACGTGAAGGGCATGTTCGAGG | |
| | domain | TGCTGGAGCCTCTGCACGCCATGATGGAGAGAGGCCCA | |
| | | CAGACCCTGAAGGAGACATCCTTTAACCAGGCCTATGG | |
| | | ACGGGACCTGATGGAGGCACAGGAGTGGTGCAGAAAGT | |
| | | ACATGAAGTCTGGCAATGTGAAGGACCTGACGCAGGCC | |
| | | TGGGATCTGTACTATCACGTGTTTCGGAGAATCTCCAA | |
| | | G | |
| | IL-2Rb | GGCAAAGACACG | 80 |
| | extracellular | | |
| | domain | | |
| | fragment | | |
| | IL-2Rb | ATTCCGTGGCTTGGGCATCTGCTCGTTGGGCTGAGTGG | 81 |
| | transmembrane | TGCGTTTGGTTTCATCATCTTGGTCTATCTCTTGATC | |
| | domain | | |
| | IL-2Rb | AATTGCAGAAATACAGGCCCTTGGCTGAAAAAAGTGCT | 82 |
| | cytoplasmic | CAAGTGTAATACCCCCGACCCAAGCAAGTTCTTCTCCC | |
| | domain | AGCTTTCTTCAGAGCATGGAGGCGATGTGCAGAAATGG | |
| | | CTCTCTTCACCTTTTCCCTCCTCAAGTTTCTCCCCGGG | |
| | | AGGGCTGGCGCCCGAGATTTCACCTCTTGAGGTACTTG | |
| | | AACGAGACAAGGTTACCCAACTTCTCCTTCAACAGGAT | |
| | | AAGGTACCCGAACCTGCGAGCCTTAGCTCCAACCACTC | |
| | | TCTTACGAGCTGCTTCACCAATCAGGGATACTTCTTTT | |
| | | TCCACCTTCCCGATGCGCTGGAAATCGAAGCATGTCAA | |
| | | GTTTACTTTACCTATGATCCATATAGCGAGGAAGATCC | |
| | | CGACGAAGGAGTCGCCGGTGCGCCCACGGGTTCCTCAC | |
| | | CCCAACCTCTCCAGCCTCTCTCAGGAGAAGATGATGCT | |
| | | TATTGCACTTTTCCCAGTAGAGACGATCTCCTCCTCTT | |
| | | TTCTCCATCTCTTTTGGGGGGACCTTCCCCCCCTTCTA | |
| | | CGGCACCTGGCGGGTCTGGTGCTGGCGAGGAGCGGATG | |
| | | CCGCCGTCCCTCCAGGAGCGAGTACCACGAGATTGGGA | |
| | | TCCCCAGCCACTTGGACCACCAACTCCAGGCGTACCTG | |
| | | ACCTTGTCGATTTTCAACCTCCCCCTGAATTGGTGCTG | |
| | | CGAGAGGCTGGGGAGGAAGTTCCGGACGCTGGGCCGAG | |
| | | GGAGGGCGTGTCCTTTCCATGGAGTAGGCCTCCAGGTC | |
| | | AAGGCGAGTTTAGGGCTCTCAACGCGCGGCTGCCGTTG | |
| | | AATACAGACGCTTATCTCTCACTGCAGGAACTGCAAGG | |
| | | TCAGGACCCAACACATCTTGTA | |
| | FKBP-IL2Rg | GAGATGTGGCACGAGGGCCTGGAGGAGGCCAGCAGGCT | 83 |
| | (full-length) | GTATTTTGGCGAGCGCAACGTGAAGGGCATGTTCGAGG | |
| | | TGCTGGAGCCTCTGCACGCCATGATGGAGAGAGGCCCA | |
| | | CAGACCCTGAAGGAGACATCCTTTAACCAGGCCTATGG | |
| | | ACGGGACCTGATGGAGGCACAGGAGTGGTGCAGAAAGT | |
| | | ACATGAAGTCTGGCAATGTGAAGGACCTGACGCAGGCC | |
| | | TGGGATCTGTACTATCACGTGTTTCGGAGAATCTCCAA | |
| | | GGGCAAAGACACGATTCCGTGGCTTGGGCATCTGCTCG | |
| | | TTGGGCTGAGTGGTGCGTTTGGTTTCATCATCTTGGTC | |
| | | TATCTCTTGATCAATTGCAGAAATACAGGCCCTTGGCT | |
| | | GAAAAAAGTGCTCAAGTGTAATACCCCCGACCCAAGCA | |
| | | AGTTCTTCTCCCAGCTTTCTTCAGAGCATGGAGGCGAT | |
| | | GTGCAGAAATGGCTCTCTTCACCTTTTCCCTCCTCAAG | |
| | | TTTCTCCCCGGGAGGGCTGGCGCCCGAGATTTCACCTC | |
| | | TTGAGGTACTTGAACGAGACAAGGTTACCCAACTTCTC | |
| | | CTTCAACAGGATAAGGTACCCGAACCTGCGAGCCTTAG | |
| | | CTCCAACCACTCTCTTACGAGCTGCTTCACCAATCAGG | |
| | | GATACTTCTTTTTCCACCTTCCCGATGCGCTGGAAATC | |
| | | GAAGCATGTCAAGTTTACTTTACCTATGATCCATATAG | |
| | | CGAGGAAGATCCCGACGAAGGAGTCGCCGGTGCGCCCA | |
| | | CGGGTTCCTCACCCCAACCTCTCCAGCCTCTCTCAGGA | |
| | | GAAGATGATGCTTATTGCACTTTTCCCAGTAGAGACGA | |
| | | TCTCCTCCTCTTTTCTCCATCTCTTTTGGGGGGACCTT | |
| | | CCCCCCCTTCTACGGCACCTGGCGGGTCTGGTGCTGGC | |
| | | GAGGAGCGGATGCCGCCGTCCCTCCAGGAGCGAGTACC | |
| | | ACGAGATTGGGATCCCCAGCCACTTGGACCACCAACTC | |
| | | CAGGCGTACCTGACCTTGTCGATTTTCAACCTCCCCCT | |
| | | GAATTGGTGCTGCGAGAGGCTGGGGAGGAAGTTCCGGA | |
| | | CGCTGGGCCGAGGGAGGGCGTGTCCTTTCCATGGAGTA | |
| | | GGCCTCCAGGTCAAGGCGAGTTTAGGGCTCTCAACGCG | |
| | | CGGCTGCCGTTGAATACAGACGCTTATCTCTCACTGCA | |
| | | GGAACTGCAAGGTCAGGACCCAACACATCTTGTA | |
| Cytosolic | Cytosolic FRB | GAAATGTGGCACGAAGGACTCGAGGAAGCCAGTCGGCT | 84 |
| FRB domain | domain | GTATTTTGGCGAGCGGAATGTGAAAGGGATGTTTGAAG | |
| | | TGCTCGAGCCTCTCCACGCTATGATGGAACGGGGACCC | |
| | | CAGACTCTCAAAGAAACCAGCTTTAATCAGGCTTACGG | |
| | | ACGCGACCTCATGGAAGCTCAAGAATGGTGTAGAAAGT | |
| | | ATATGAAGAGTGGCAACGTGAAAGATCTGACACAAGCC | |
| | | TGGGATCTCTATTATCACGTGTTCAGACGCATCAGCAA | |
| | | A | |

TABLE 5
Examples of nucleic acids for insertion into a TRAC locus

| Nucleic | Encoded | | SEQ |
|---|---|---|---|
| Acid | Sequence | | ID |
| # | Name | Nucleotide Sequence | NO: |
| T1D2_v1 | 5′ Homology | CCTATTAAATAAAAGAATAAGCAGTATTATTAAGTAGCCCTGC | 85 |
| | Arm | ATTTCAGGTTTCCTTGAGTGGCAGGCCAGGCCTGGCCGTGAAC | |
| | | GTTCACTGAAATCATGGCCTCTTGGCCAAGATTGATAGCTTGT | |
| | | GCCTGTCCCTGAGTCCCAGTCCATCACGAGCAGCTGGTTTCTA | |
| | | AGATGCTATTTCCCGTATAAAGCATGAGACCGTGACTTGCCAG | |
| | | CCCCACAGAGCCCCGCCCTTGTCCATCACTGGCATCTGGACTC | |
| | | CAGCCTGGGTTGGGGCAAAGAGGGAAATGAGATCATGTCCTAA | |
| | | CCCTGATCCTCTTGTCCCACAGATATCCAGAACCCTGACCCTG | |
| | | CC | |
| | Intervening | TA | |
| | sequence | | |
| | MND | GAACAGAGAAACAGGAGAATATGGGCCAAACAGGATATCTGTG | 86 |
| | Promoter | GTAAGCAGTTCCTGCCCCGGCTCAGGGCCAAGAACAGTTGGAA | |
| | | CAGCAGAATATGGGCCAAACAGGATATCTGTGGTAAGCAGTTC | |
| | | CTGCCCCGGCTCAGGGCCAAGAACAGATGGTCCCCAGATGCGG | |
| | | TCCCGCCCTCAGCAGTTTCTAGAGAACCATCAGATGTTTCCAG | |
| | | GGTGCCCCAAGGACCTGAAATGACCCTGTGCCTTATTTGAACT | |
| | | AACCAATCAGTTCGCTTCTCGCTTCTGTTCGCGCGCTTCTGCT | |
| | | CCCCGAGCTCTATATAAGCAGAGCTCGTTTAGTGAACCGTCAG | |
| | | ATC | |
| | Intervening | GCCTGGAGACGCCATCCACGCTGTTTTGACTTCCATAGAAGGA | 87 |
| | Sequence | TCTCGCCGCCACCATGCCTCTGGGCCTGCTGTGGCTGGGCCTG | |
| | (LCN2 | GCCCTGCTGGGCGCCCTGCACGCCCAGGCC | |
| | signal | | |
| | peptide of | | |
| | FKBP-IL2Rg | | |
| | underlined | | |
| | FKBP-IL2Rg | GGCGTGCAGGTGGAGACAATCTCCCCAGGCGACGGACGCACAT | 88 |
| | | TCCCTAAGCGGGGCCAGACCTGCGTGGTGCACTATACAGGCAT | |
| | | GCTGGAGGATGGCAAGAAGTTTGACAGCTCCCGGGATAGAAAC | |
| | | AAGCCATTCAAGTTTATGCTGGGCAAGCAGGAAGTGATCAGAG | |
| | | GCTGGGAGGAGGGCGTGGCCCAGATGTCTGTGGGCCAGAGGGC | |
| | | CAAGCTGACCATCAGCCCAGACTACGCCTATGGAGCAACAGGC | |
| | | CACCCAGGAATCATCCCACCTCACGCCACCCTGGTGTTCGATG | |
| | | TGGAGCTGCTGAAGCTGGGCGAGGGATCCAACACATCAAAAGA | |
| | | GAACCCCTTTCTGTTCGCATTGGAGGCCGTAGTCATATCTGTT | |
| | | GGATCCATGGGACTTATTATCTCCCTGTTGTGTGTGTACTTCT | |
| | | GGCTGGAACGGACTATGCCCAGGATCCCCACGCTCAAGAATCT | |
| | | AGCGGAGTCTCTAAGGGTCTGGCCGAATCCCTCCAACCCGATT | |
| | | ATTCTGAACGGTTGTGCCTCGTATCCGAAATACCACCAAAAGG | |
| | | CGGGGCTCTGGGTGAGGGCCCAGGGGCGAGTCCGTGCAATCAA | |
| | | CACAGCCCGTATTGGGCCCCTCCTTGTTATACGTTGAAGCCCG | |
| | | AAACT | |
| | GSG linker | GGATCCGGC | |
| | T2A | GAGGGCAGAGGAAGTCTGCTAACATGCGGTGACGTCGAGGAGA | 89 |
| | | ATCCTGGACCT | |
| | T1D2 TCRβ | ATGAGCATCAGCCTCCTGTGCTGTGCAGCCTTTCCTCTCCTGT | 90 |
| | | GGGCAGGTCCAGTGAATGCTGGTGTCACTCAGACCCCAAAATT | |
| | | CCGCATCCTGAAGATAGGACAGAGCATGACACTGCAGTGTACC | |
| | | CAGGATATGAACCATAACTACATGTACTGGTATCGACAAGACC | |
| | | CAGGCATGGGGCTGAAGCTGATTTATTATTCAGTTGGTGCTGG | |
| | | TATCACTGATAAAGGAGAAGTCCCGAATGGCTACAACGTCTCC | |
| | | AGATCAACCACAGAGGATTTCCCGCTCAGGCTGGAGTTGGCTG | |
| | | CTCCCTCCCAGACATCTGTGTACTTCTGTGCCAGCAGTCCGTG | |
| | | GGGGGCAGGCGGCACAGATACGCAGTATTTTGGCCCAGGCACC | |
| | | CGGCTGACAGTGCTCGAGGACCTTAAGAATGTGTTCCCTCCCG | |
| | | AGGTGGCTGTCTTCGAACCAAGCGAAGCCGAGATCTCTCACAC | |
| | | ACAAAAGGCTACTCTCGTGTGTCTGGCAACGGGTTTTTACCCT | |
| | | GATCATGTAGAGTTGTCTTGGTGGGTTAACGGCAAAGAAGTAC | |
| | | ACTCCGGTGTAAGCACCGATCCTCAGCCACTTAAGGAACAACC | |
| | | CGCACTCAACGACTCCAGATACTGTTTGAGTTCTAGGCTGAGA | |
| | | GTCTCAGCGACGTTTTGGCAGAACCCACGGAATCATTTCAGAT | |
| | | GTCAGGTCCAGTTTTACGGGTTGAGCGAGAACGACGAGTGGAC | |
| | | ACAGGATCGGGCTAAACCAGTGACCCAGATTGTATCAGCCGAA | |
| | | GCATGGGGAAGGGCAGATTGCGGCTTTACCTCAGAGTCATATC | |
| | | AACAGGGCGTTTTGTCTGCAACAATCCTTTATGAGATCCTGCT | |
| | | TGGTAAGGCGACTCTGTATGCCGTGCTTGTTAGTGCCCTGGTT | |
| | | CTGATGGCAATGGTAAAAAGAAAGGACTCTAGGGGA | |
| | GSG linker | GGTTCTGGC | |
| | P2A | GCTACTAACTTCAGCCTGCTGAAGCAGGCTGGAGACGTGGAGG | 91 |
| | | AGAACCCTGGACCT | |
| | T1D2 TCRα | ATGGCCATGCTCCTGGGGGCATCAGTGCTGATTCTGTGGCTTC | 92 |
| | Vα domain | AGCCAGACTGGGTAAACAGTCAACAGAAGAATGATGACCAGCA | |
| | and C | AGTTAAGCAAAATTCACCATCCCTGAGCGTCCAGGAAGGAAGA | |
| | domain | ATTTCTATTCTGAACTGTGACTATACTAACAGCATGTTTGATT | |
| | portion | ATTTCCTATGGTACAAAAAATACCCTGCTGAAGGTCCTACATT | |
| | | CCTGATATCTATAAGTTCCATTAAGGATAAAAATGAAGATGGA | |
| | | AGATTCACTGTCTTCTTAAACAAAAGTGCCAAGCACCTCTCTC | |
| | | TGCACATTGTGCCCTCCCAGCCTGGAGACTCTGCAGTGTACTT | |
| | | CTGTGCAGCAACCCGTACCTCAGGAACCTACAAATACATCTTT | |
| | | GGAACAGGCACCAGGCTGAAGGTTTTAGCAAATATCCAGAACC | |
| | | CTGACCCTGCAGTATAT | |
| | 3′ Homology | CAGCTGAGAGACTCTAAATCCAGTGACAAGTCTGTCTGCCTAT | 93 |
| | Arm | TCACCGATTTTGATTCTCAAACAAATGTGTCACAAAGTAAGGA | |
| | | TTCTGATGTGTATATCACAGACAAAACTGTGCTAGACATGAGG | |
| | | TCTATGGACTTCAAGAGCAACAGTGCTGTGGCCTGGAGCAACA | |
| | | AATCTGACTTTGCATGTGCAAACGCCTTCAACAACAGCATTAT | |
| | | TCCAGAAGACACCTTCTTCCCCAGCCCAGGTAAGGGCAGCTTT | |
| | | GGTGCCTTCGCAGGCTGTTTCCTTGCTTCAGGAATGGCCAGGT | |
| | | TCTGCCCAGAGCTCTGGTCAATGATGTCTAAAACTCCTCTGAT | |
| | | TGGTGGTCTCGGC | |
| | Between | TAGAACAGAGAAACAGGAGAATATGGGCCAAACAGGATATCTG | 94 |
| | Homology | TGGTAAGCAGTTCCTGCCCCGGCTCAGGGCCAAGAACAGTTGG | |
| | Arms | AACAGCAGAATATGGGCCAAACAGGATATCTGTGGTAAGCAGT | |
| | | TCCTGCCCCGGCTCAGGGCCAAGAACAGATGGTCCCCAGATGC | |
| | | GGTCCCGCCCTCAGCAGTTTCTAGAGAACCATCAGATGTTTCC | |
| | | AGGGTGCCCCAAGGACCTGAAATGACCCTGTGCCTTATTTGAA | |
| | | CTAACCAATCAGTTCGCTTCTCGCTTCTGTTCGCGCGCTTCTG | |
| | | CTCCCCGAGCTCTATATAAGCAGAGCTCGTTTAGTGAACCGTC | |
| | | AGATCGCCTGGAGACGCCATCCACGCTGTTTTGACTTCCATAG | |
| | | AAGGATCTCGCCGCCACCATGCCTCTGGGCCTGCTGTGGCTGG | |
| | | GCCTGGCCCTGCTGGGCGCCCTGCACGCCCAGGCCGGCGTGCA | |
| | | GGTGGAGACAATCTCCCCAGGCGACGGACGCACATTCCCTAAG | |
| | | CGGGGCCAGACCTGCGTGGTGCACTATACAGGCATGCTGGAGG | |
| | | ATGGCAAGAAGTTTGACAGCTCCCGGGATAGAAACAAGCCATT | |
| | | CAAGTTTATGCTGGGCAAGCAGGAAGTGATCAGAGGCTGGGAG | |
| | | GAGGGCGTGGCCCAGATGTCTGTGGGCCAGAGGGCCAAGCTGA | |
| | | CCATCAGCCCAGACTACGCCTATGGAGCAACAGGCCACCCAGG | |
| | | AATCATCCCACCTCACGCCACCCTGGTGTTCGATGTGGAGCTG | |
| | | CTGAAGCTGGGCGAGGGATCCAACACATCAAAAGAGAACCCCT | |
| | | TTCTGTTCGCATTGGAGGCCGTAGTCATATCTGTTGGATCCAT | |
| | | GGGACTTATTATCTCCCTGTTGTGTGTGTACTTCTGGCTGGAA | |
| | | CGGACTATGCCCAGGATCCCCACGCTCAAGAATCTGGAAGATC | |
| | | TCGTCACAGAATACCATGGTAATTTCAGCGCCTGGAGCGGAGT | |
| | | CTCTAAGGGTCTGGCCGAATCCCTCCAACCCGATTATTCTGAA | |
| | | CGGTTGTGCCTCGTATCCGAAATACCACCAAAAGGCGGGGCTC | |
| | | TGGGTGAGGGCCCAGGGGCGAGTCCGTGCAATCAACACAGCCC | |
| | | GTATTGGGCCCCTCCTTGTTATACGTTGAAGCCCGAAACTGGA | |
| | | TCCGGCGAGGGCAGAGGAAGTCTGCTAACATGCGGTGACGTCG | |
| | | AGGAGAATCCTGGACCTATGAGCATCAGCCTCCTGTGCTGTGC | |
| | | AGCCTTTCCTCTCCTGTGGGCAGGTCCAGTGAATGCTGGTGTC | |
| | | ACTCAGACCCCAAAATTCCGCATCCTGAAGATAGGACAGAGCA | |
| | | TGACACTGCAGTGTACCCAGGATATGAACCATAACTACATGTA | |
| | | CTGGTATCGACAAGACCCAGGCATGGGGCTGAAGCTGATTTAT | |
| | | TATTCAGTTGGTGCTGGTATCACTGATAAAGGAGAAGTCCCGA | |
| | | ATGGCTACAACGTCTCCAGATCAACCACAGAGGATTTCCCGCT | |
| | | CAGGCTGGAGTTGGCTGCTCCCTCCCAGACATCTGTGTACTTC | |
| | | TGTGCCAGCAGTCCGTGGGGGGCAGGCGGCACAGATACGCAGT | |
| | | ATTTTGGCCCAGGCACCCGGCTGACAGTGCTCGAGGACCTTAA | |
| | | GAATGTGTTCCCTCCCGAGGTGGCTGTCTTCGAACCAAGCGAA | |
| | | GCCGAGATCTCTCACACACAAAAGGCTACTCTCGTGTGTCTGG | |
| | | CAACGGGTTTTTACCCTGATCATGTAGAGTTGTCTTGGTGGGT | |
| | | TAACGGCAAAGAAGTACACTCCGGTGTAAGCACCGATCCTCAG | |
| | | CCACTTAAGGAACAACCCGCACTCAACGACTCCAGATACTGTT | |
| | | TGAGTTCTAGGCTGAGAGTCTCAGCGACGTTTTGGCAGAACCC | |
| | | ACGGAATCATTTCAGATGTCAGGTCCAGTTTTACGGGTTGAGC | |
| | | GAGAACGACGAGTGGACACAGGATCGGGCTAAACCAGTGACCC | |
| | | AGATTGTATCAGCCGAAGCATGGGGAAGGGCAGATTGCGGCTT | |
| | | TACCTCAGAGTCATATCAACAGGGCGTTTTGTCTGCAACAATC | |
| | | CTTTATGAGATCCTGCTTGGTAAGGCGACTCTGTATGCCGTGC | |
| | | TTGTTAGTGCCCTGGTTCTGATGGCAATGGTAAAAAGAAAGGA | |
| | | CTCTAGGGGAGGTTCTGGCGCTACTAACTTCAGCCTGCTGAAG | |
| | | CAGGCTGGAGACGTGGAGGAGAACCCTGGACCTATGGCCATGC | |
| | | TCCTGGGGGCATCAGTGCTGATTCTGTGGCTTCAGCCAGACTG | |
| | | GGTAAACAGTCAACAGAAGAATGATGACCAGCAAGTTAAGCAA | |
| | | AATTCACCATCCCTGAGCGTCCAGGAAGGAAGAATTTCTATTC | |
| | | TGAACTGTGACTATACTAACAGCATGTTTGATTATTTCCTATG | |
| | | GTACAAAAAATACCCTGCTGAAGGTCCTACATTCCTGATATCT | |
| | | ATAAGTTCCATTAAGGATAAAAATGAAGATGGAAGATTCACTG | |
| | | TCTTCTTAAACAAAAGTGCCAAGCACCTCTCTCTGCACATTGT | |
| | | GCCCTCCCAGCCTGGAGACTCTGCAGTGTACTTCTGTGCAGCA | |
| | | ACCCGTACCTCAGGAACCTACAAATACATCTTTGGAACAGGCA | |
| | | CCAGGCTGAAGGTTTTAGCAAATATCCAGAACCCTGACCCTGC | |
| | | AGTATAT | |
| | Full-length | CCTATTAAATAAAAGAATAAGCAGTATTATTAAGTAGCCCTGC | 95 |
| | donor | ATTTCAGGTTTCCTTGAGTGGCAGGCCAGGCCTGGCCGTGAAC | |
| | template | GTTCACTGAAATCATGGCCTCTTGGCCAAGATTGATAGCTTGT | |
| | | GCCTGTCCCTGAGTCCCAGTCCATCACGAGCAGCTGGTTTCTA | |
| | | AGATGCTATTTCCCGTATAAAGCATGAGACCGTGACTTGCCAG | |
| | | CCCCACAGAGCCCCGCCCTTGTCCATCACTGGCATCTGGACTC | |
| | | CAGCCTGGGTTGGGGCAAAGAGGGAAATGAGATCATGTCCTAA | |
| | | CCCTGATCCTCTTGTCCCACAGATATCCAGAACCCTGACCCTG | |
| | | CCTAGAACAGAGAAACAGGAGAATATGGGCCAAACAGGATATC | |
| | | TGTGGTAAGCAGTTCCTGCCCCGGCTCAGGGCCAAGAACAGTT | |
| | | GGAACAGCAGAATATGGGCCAAACAGGATATCTGTGGTAAGCA | |
| | | GTTCCTGCCCCGGCTCAGGGCCAAGAACAGATGGTCCCCAGAT | |
| | | GCGGTCCCGCCCTCAGCAGTTTCTAGAGAACCATCAGATGTTT | |
| | | CCAGGGTGCCCCAAGGACCTGAAATGACCCTGTGCCTTATTTG | |
| | | AACTAACCAATCAGTTCGCTTCTCGCTTCTGTTCGCGCGCTTC | |
| | | TGCTCCCCGAGCTCTATATAAGCAGAGCTCGTTTAGTGAACCG | |
| | | TCAGATCGCCTGGAGACGCCATCCACGCTGTTTTGACTTCCAT | |
| | | AGAAGGATCTCGCCGCCACCATGCCTCTGGGCCTGCTGTGGCT | |
| | | GGGCCTGGCCCTGCTGGGCGCCCTGCACGCCCAGGCCGGCGTG | |
| | | CAGGTGGAGACAATCTCCCCAGGCGACGGACGCACATTCCCTA | |
| | | AGCGGGGCCAGACCTGCGTGGTGCACTATACAGGCATGCTGGA | |
| | | GGATGGCAAGAAGTTTGACAGCTCCCGGGATAGAAACAAGCCA | |
| | | TTCAAGTTTATGCTGGGCAAGCAGGAAGTGATCAGAGGCTGGG | |
| | | AGGAGGGCGTGGCCCAGATGTCTGTGGGCCAGAGGGCCAAGCT | |
| | | GACCATCAGCCCAGACTACGCCTATGGAGCAACAGGCCACCCA | |
| | | GGAATCATCCCACCTCACGCCACCCTGGTGTTCGATGTGGAGC | |
| | | TGCTGAAGCTGGGCGAGGGATCCAACACATCAAAAGAGAACCC | |
| | | CTTTCTGTTCGCATTGGAGGCCGTAGTCATATCTGTTGGATCC | |
| | | ATGGGACTTATTATCTCCCTGTTGTGTGTGTACTTCTGGCTGG | |
| | | AACGGACTATGCCCAGGATCCCCACGCTCAAGAATCTGGAAGA | |
| | | TCTCGTCACAGAATACCATGGTAATTTCAGCGCCTGGAGCGGA | |
| | | GTCTCTAAGGGTCTGGCCGAATCCCTCCAACCCGATTATTCTG | |
| | | AACGGTTGTGCCTCGTATCCGAAATACCACCAAAAGGCGGGGC | |
| | | TCTGGGTGAGGGCCCAGGGGCGAGTCCGTGCAATCAACACAGC | |
| | | CCGTATTGGGCCCCTCCTTGTTATACGTTGAAGCCCGAAACTG | |
| | | GATCCGGCGAGGGCAGAGGAAGTCTGCTAACATGCGGTGACGT | |
| | | CGAGGAGAATCCTGGACCTATGAGCATCAGCCTCCTGTGCTGT | |
| | | GCAGCCTTTCCTCTCCTGTGGGCAGGTCCAGTGAATGCTGGTG | |
| | | TCACTCAGACCCCAAAATTCCGCATCCTGAAGATAGGACAGAG | |
| | | CATGACACTGCAGTGTACCCAGGATATGAACCATAACTACATG | |
| | | TACTGGTATCGACAAGACCCAGGCATGGGGCTGAAGCTGATTT | |
| | | ATTATTCAGTTGGTGCTGGTATCACTGATAAAGGAGAAGTCCC | |
| | | GAATGGCTACAACGTCTCCAGATCAACCACAGAGGATTTCCCG | |
| | | CTCAGGCTGGAGTTGGCTGCTCCCTCCCAGACATCTGTGTACT | |
| | | TCTGTGCCAGCAGTCCGTGGGGGGCAGGCGGCACAGATACGCA | |
| | | GTATTTTGGCCCAGGCACCCGGCTGACAGTGCTCGAGGACCTT | |
| | | AAGAATGTGTTCCCTCCCGAGGTGGCTGTCTTCGAACCAAGCG | |
| | | AAGCCGAGATCTCTCACACACAAAAGGCTACTCTCGTGTGTCT | |
| | | GGCAACGGGTTTTTACCCTGATCATGTAGAGTTGTCTTGGTGG | |
| | | GTTAACGGCAAAGAAGTACACTCCGGTGTAAGCACCGATCCTC | |
| | | AGCCACTTAAGGAACAACCCGCACTCAACGACTCCAGATACTG | |
| | | TTTGAGTTCTAGGCTGAGAGTCTCAGCGACGTTTTGGCAGAAC | |
| | | CCACGGAATCATTTCAGATGTCAGGTCCAGTTTTACGGGTTGA | |
| | | GCGAGAACGACGAGTGGACACAGGATCGGGCTAAACCAGTGAC | |
| | | CCAGATTGTATCAGCCGAAGCATGGGGAAGGGCAGATTGCGGC | |
| | | TTTACCTCAGAGTCATATCAACAGGGCGTTTTGTCTGCAACAA | |
| | | TCCTTTATGAGATCCTGCTTGGTAAGGCGACTCTGTATGCCGT | |
| | | GCTTGTTAGTGCCCTGGTTCTGATGGCAATGGTAAAAAGAAAG | |
| | | GACTCTAGGGGAGGTTCTGGCGCTACTAACTTCAGCCTGCTGA | |
| | | AGCAGGCTGGAGACGTGGAGGAGAACCCTGGACCTATGGCCAT | |
| | | GCTCCTGGGGGCATCAGTGCTGATTCTGTGGCTTCAGCCAGAC | |
| | | TGGGTAAACAGTCAACAGAAGAATGATGACCAGCAAGTTAAGC | |
| | | AAAATTCACCATCCCTGAGCGTCCAGGAAGGAAGAATTTCTAT | |
| | | TCTGAACTGTGACTATACTAACAGCATGTTTGATTATTTCCTA | |
| | | TGGTACAAAAAATACCCTGCTGAAGGTCCTACATTCCTGATAT | |
| | | CTATAAGTTCCATTAAGGATAAAAATGAAGATGGAAGATTCAC | |
| | | TGTCTTCTTAAACAAAAGTGCCAAGCACCTCTCTCTGCACATT | |
| | | GTGCCCTCCCAGCCTGGAGACTCTGCAGTGTACTTCTGTGCAG | |
| | | CAACCCGTACCTCAGGAACCTACAAATACATCTTTGGAACAGG | |
| | | CACCAGGCTGAAGGTTTTAGCAAATATCCAGAACCCTGACCCT | |
| | | GCAGTATATCAGCTGAGAGACTCTAAATCCAGTGACAAGTCTG | |
| | | TCTGCCTATTCACCGATTTTGATTCTCAAACAAATGTGTCACA | |
| | | AAGTAAGGATTCTGATGTGTATATCACAGACAAAACTGTGCTA | |
| | | GACATGAGGTCTATGGACTTCAAGAGCAACAGTGCTGTGGCCT | |
| | | GGAGCAACAAATCTGACTTTGCATGTGCAAACGCCTTCAACAA | |
| | | CAGCATTATTCCAGAAGACACCTTCTTCCCCAGCCCAGGTAAG | |
| | | GGCAGCTTTGGTGCCTTCGCAGGCTGTTTCCTTGCTTCAGGAA | |
| | | TGGCCAGGTTCTGCCCAGAGCTCTGGTCAATGATGTCTAAAAC | |
| | | TCCTCTGATTGGTGGTCTCGGC | |
| T1D2 v2 | 5′ Homology | AACATACCATAAACCTCCCATTCTGCTAATGCCCAGCCTAAGT | 96 |
| | Arm | TGGGGAGACCACTCCAGATTCCAAGATGTACAGTTTGCTTTGC | |
| | | TGGGCCTTTTTCCCATGCCTGCCTTTACTCTGCCAGAGTTATA | |
| | | TTGCTGGGGTTTTGAAGAAGATCCTATTAAATAAAAGAATAAG | |
| | | CAGTATTATTAAGTAGCCCTGCATTTCAGGTTTCCTTGAGTGG | |
| | | CAGGCCAGGCCTGGCCGTGAACGTTCACTGAAATCATGGCCTC | |
| | | TTGGCCAAGATTGATAGCTTGTGCCTGTCCCTGAGTCCCAGTC | |
| | | CATCACGAGCAGCTGGTTTCTAAGATGCTATTTCCCGTATAAA | |
| | | GCATGAGACCGTGACTTGCCAGCCCCACAGAGCCCCGCCCTTG | |
| | | TCCATCACTGGCATCTGGACTCCAGCCTGGGTTGGGGCAAAGA | |
| | | GGGAAATGAGATCATGTCCTAACCCTGATCCTCTTGTCCCACA | |
| | | GATATCCAGAACCCTGACCCTGCCGTG | |
| | Intervening | TAGCAGCTTAGATAC | 97 |
| | sequence | | |
| | MND | GAACAGAGAAACAGGAGAATATGGGCCAAACAGGATATCTGTG | 98 |
| | Promoter | GTAAGCAGTTCCTGCCCCGGCTCAGGGCCAAGAACAGTTGGAA | |
| | | CAGCAGAATATGGGCCAAACAGGATATCTGTGGTAAGCAGTTC | |
| | | CTGCCCCGGCTCAGGGCCAAGAACAGATGGTCCCCAGATGCGG | |
| | | TCCCGCCCTCAGCAGTTTCTAGAGAACCATCAGATGTTTCCAG | |
| | | GGTGCCCCAAGGACCTGAAATGACCCTGTGCCTTATTTGAACT | |
| | | AACCAATCAGTTCGCTTCTCGCTTCTGTTCGCGCGCTTCTGCT | |
| | | CCCCGAGCTCTATATAAGCAGAGCTCGTTTAGTGAACCGTCAG | |
| | | ATC | |
| | Intervening | GCCTGGAGACGCCATCCACGCTGTTTTGACTTCCATAGAAGGA | 99 |
| | Sequence | TCTCGCCGCCACCATGCCTCTGGGCCTGCTGTGGCTGGGCCTG | |
| | (LCN2 | GCCCTGCTGGGCGCCCTGCACGCCCAGGCC | |
| | signal | | |
| | peptide of | | |
| | FKBP-IL2Rg | | |
| | underlined | | |
| | FKBP-IL2Rg | GGCGTGCAGGTGGAGACAATCTCCCCAGGCGACGGACGCACAT | 100 |
| | | TCCCTAAGCGGGGCCAGACCTGCGTGGTGCACTATACAGGCAT | |
| | | GCTGGAGGATGGCAAGAAGTTTGACAGCTCCCGGGATAGAAAC | |
| | | AAGCCATTCAAGTTTATGCTGGGCAAGCAGGAAGTGATCAGAG | |
| | | GCTGGGAGGAGGGCGTGGCCCAGATGTCTGTGGGCCAGAGGGC | |
| | | CAAGCTGACCATCAGCCCAGACTACGCCTATGGAGCAACAGGC | |
| | | CACCCAGGAATCATCCCACCTCACGCCACCCTGGTGTTCGATG | |
| | | TGGAGCTGCTGAAGCTGGGCGAGGGATCCAACACATCAAAAGA | |
| | | GAACCCCTTTCTGTTCGCATTGGAGGCCGTAGTCATATCTGTT | |
| | | GGATCCATGGGACTTATTATCTCCCTGTTGTGTGTGTACTTCT | |
| | | GGCTGGAACGGACTATGCCCAGGATCCCCACGCTCAAGAATCT | |
| | | GGAAGATCTCGTCACAGAATACCATGGTAATTTCAGCGCCTGG | |
| | | AGCGGAGTCTCTAAGGGTCTGGCCGAATCCCTCCAACCCGATT | |
| | | ATTCTGAACGGTTGTGCCTCGTATCCGAAATACCACCAAAAGG | |
| | | CGGGGCTCTGGGTGAGGGCCCAGGGGCGAGTCCGTGCAATCAA | |
| | | CACAGCCCGTATTGGGCCCCTCCTTGTTATACGTTGAAGCCCG | |
| | | AAACT | |
| | GSG linker | GGATCCGGC | |
| | T2A | GAGGGCAGAGGAAGTCTGCTAACATGCGGTGACGTCGAGGAGA | 101 |
| | | ATCCTGGACCT | |
| | T1D2 TCRβ | ATGAGCATCAGCCTCCTGTGCTGTGCAGCCTTTCCTCTCCTGT | 102 |
| | | GGGCAGGTCCAGTGAATGCTGGTGTCACTCAGACCCCAAAATT | |
| | | CCGCATCCTGAAGATAGGACAGAGCATGACACTGCAGTGTACC | |
| | | CAGGATATGAACCATAACTACATGTACTGGTATCGACAAGACC | |
| | | CAGGCATGGGGCTGAAGCTGATTTATTATTCAGTTGGTGCTGG | |
| | | TATCACTGATAAAGGAGAAGTCCCGAATGGCTACAACGTCTCC | |
| | | AGATCAACCACAGAGGATTTCCCGCTCAGGCTGGAGTTGGCTG | |
| | | CTCCCTCCCAGACATCTGTGTACTTCTGTGCCAGCAGTCCGTG | |
| | | GGGGGCAGGCGGCACAGATACGCAGTATTTTGGCCCAGGCACC | |
| | | CGGCTGACAGTGCTCGAGGACCTTAAGAATGTGTTCCCTCCCG | |
| | | AGGTGGCTGTCTTCGAACCAAGCGAAGCCGAGATCTCTCACAC | |
| | | ACAAAAGGCTACTCTCGTGTGTCTGGCAACGGGTTTTTACCCT | |
| | | GATCATGTAGAGTTGTCTTGGTGGGTTAACGGCAAAGAAGTAC | |
| | | ACTCCGGTGTAAGCACCGATCCTCAGCCACTTAAGGAACAACC | |
| | | CGCACTCAACGACTCCAGATACTGTTTGAGTTCTAGGCTGAGA | |
| | | GTCTCAGCGACGTTTTGGCAGAACCCACGGAATCATTTCAGAT | |
| | | GTCAGGTCCAGTTTTACGGGTTGAGCGAGAACGACGAGTGGAC | |
| | | ACAGGATCGGGCTAAACCAGTGACCCAGATTGTATCAGCCGAA | |
| | | GCATGGGGAAGGGCAGATTGCGGCTTTACCTCAGAGTCATATC | |
| | | AACAGGGCGTTTTGTCTGCAACAATCCTTTATGAGATCCTGCT | |
| | | TGGTAAGGCGACTCTGTATGCCGTGCTTGTTAGTGCCCTGGTT | |
| | | CTGATGGCAATGGTAAAAAGAAAGGACTCTAGGGGA | |
| | GSG linker | GGTTCTGGC | |
| | P2A | GCTACTAACTTCAGCCTGCTGAAGCAGGCTGGAGACGTGGAGG | 103 |
| | | AGAACCCTGGACCT | |
| | T1D2 TCRα | ATGGCCATGCTCCTGGGGGCATCAGTGCTGATTCTGTGGCTTC | 104 |
| | Vα domain | AGCCAGACTGGGTAAACAGTCAACAGAAGAATGATGACCAGCA | |
| | and C | AGTTAAGCAAAATTCACCATCCCTGAGCGTCCAGGAAGGAAGA | |
| | domain | ATTTCTATTCTGAACTGTGACTATACTAACAGCATGTTTGATT | |
| | portion | ATTTCCTATGGTACAAAAAATACCCTGCTGAAGGTCCTACATT | |
| | | CCTGATATCTATAAGTTCCATTAAGGATAAAAATGAAGATGGA | |
| | | AGATTCACTGTCTTCTTAAACAAAAGTGCCAAGCACCTCTCTC | |
| | | TGCACATTGTGCCCTCCCAGCCTGGAGACTCTGCAGTGTACTT | |
| | | CTGTGCAGCAACCCGTACCTCAGGAACCTACAAATACATCTTT | |
| | | GGAACAGGCACCAGGCTGAAGGTTTTAGCAAATATCCAGAACC | |
| | | CTGACCCTGCAGTATATCAGCTGAGAGAC | |
| | 3′ Homology | TCTAAATCCAGTGACAAGTCTGTCTGCCTATTCACCGATTTTG | 105 |
| | Arm | ATTCTCAAACAAATGTGTCACAAAGTAAGGATTCTGATGTGTA | |
| | | TATCACAGACAAAACTGTGCTAGACATGAGGTCTATGGACTTC | |
| | | AAGAGCAACAGTGCTGTGGCCTGGAGCAACAAATCTGACTTTG | |
| | | CATGTGCAAACGCCTTCAACAACAGCATTATTCCAGAAGACAC | |
| | | CTTCTTCCCCAGCCCAGGTAAGGGCAGCTTTGGTGCCTTCGCA | |
| | | GGCTGTTTCCTTGCTTCAGGAATGGCCAGGTTCTGCCCAGAGC | |
| | | TCTGGTCAATGATGTCTAAAACTCCTCTGATTGGTGGTCTCGG | |
| | | CCTTATCCATTGCCACCAAAACCCTCTTTTTACTAAGAAACAG | |
| | | TGAGCCTTGTTCTGGCAGTCCAGAGAATGACACGGGAAAAAAG | |
| | | CAGATGAAGAGAAGGTGGCAGGAGAGGGCACGTGGCCCAGCCT | |
| | | CAGTCTCTCCAACTGAGTTCCTGCCTG | |
| | Between | TAGCAGCTTAGATACGAACAGAGAAACAGGAGAATATGGGCCA | 106 |
| | Homology | AACAGGATATCTGTGGTAAGCAGTTCCTGCCCCGGCTCAGGGC | |
| | Arms | CAAGAACAGTTGGAACAGCAGAATATGGGCCAAACAGGATATC | |
| | | TGTGGTAAGCAGTTCCTGCCCCGGCTCAGGGCCAAGAACAGAT | |
| | | GGTCCCCAGATGCGGTCCCGCCCTCAGCAGTTTCTAGAGAACC | |
| | | ATCAGATGTTTCCAGGGTGCCCCAAGGACCTGAAATGACCCTG | |
| | | TGCCTTATTTGAACTAACCAATCAGTTCGCTTCTCGCTTCTGT | |
| | | TCGCGCGCTTCTGCTCCCCGAGCTCTATATAAGCAGAGCTCGT | |
| | | TTAGTGAACCGTCAGATCGCCTGGAGACGCCATCCACGCTGTT | |
| | | TTGACTTCCATAGAAGGATCTCGCCGCCACCATGCCTCTGGGC | |
| | | CTGCTGTGGCTGGGCCTGGCCCTGCTGGGCGCCCTGCACGCCC | |
| | | AGGCCGGCGTGCAGGTGGAGACAATCTCCCCAGGCGACGGACG | |
| | | CACATTCCCTAAGCGGGGCCAGACCTGCGTGGTGCACTATACA | |
| | | GGCATGCTGGAGGATGGCAAGAAGTTTGACAGCTCCCGGGATA | |
| | | GAAACAAGCCATTCAAGTTTATGCTGGGCAAGCAGGAAGTGAT | |
| | | CAGAGGCTGGGAGGAGGGCGTGGCCCAGATGTCTGTGGGCCAG | |
| | | AGGGCCAAGCTGACCATCAGCCCAGACTACGCCTATGGAGCAA | |
| | | CAGGCCACCCAGGAATCATCCCACCTCACGCCACCCTGGTGTT | |
| | | CGATGTGGAGCTGCTGAAGCTGGGCGAGGGATCCAACACATCA | |
| | | AAAGAGAACCCCTTTCTGTTCGCATTGGAGGCCGTAGTCATAT | |
| | | CTGTTGGATCCATGGGACTTATTATCTCCCTGTTGTGTGTGTA | |
| | | CTTCTGGCTGGAACGGACTATGCCCAGGATCCCCACGCTCAAG | |
| | | AATCTGGAAGATCTCGTCACAGAATACCATGGTAATTTCAGCG | |
| | | CCTGGAGCGGAGTCTCTAAGGGTCTGGCCGAATCCCTCCAACC | |
| | | CGATTATTCTGAACGGTTGTGCCTCGTATCCGAAATACCACCA | |
| | | AAAGGCGGGGCTCTGGGTGAGGGCCCAGGGGCGAGTCCGTGCA | |
| | | ATCAACACAGCCCGTATTGGGCCCCTCCTTGTTATACGTTGAA | |
| | | GCCCGAAACTGGATCCGGCGAGGGCAGAGGAAGTCTGCTAACA | |
| | | TGCGGTGACGTCGAGGAGAATCCTGGACCTATGAGCATCAGCC | |
| | | TCCTGTGCTGTGCAGCCTTTCCTCTCCTGTGGGCAGGTCCAGT | |
| | | GAATGCTGGTGTCACTCAGACCCCAAAATTCCGCATCCTGAAG | |
| | | ATAGGACAGAGCATGACACTGCAGTGTACCCAGGATATGAACC | |
| | | ATAACTACATGTACTGGTATCGACAAGACCCAGGCATGGGGCT | |
| | | GAAGCTGATTTATTATTCAGTTGGTGCTGGTATCACTGATAAA | |
| | | GGAGAAGTCCCGAATGGCTACAACGTCTCCAGATCAACCACAG | |
| | | AGGATTTCCCGCTCAGGCTGGAGTTGGCTGCTCCCTCCCAGAC | |
| | | ATCTGTGTACTTCTGTGCCAGCAGTCCGTGGGGGGCAGGCGGC | |
| | | ACAGATACGCAGTATTTTGGCCCAGGCACCCGGCTGACAGTGC | |
| | | TCGAGGACCTTAAGAATGTGTTCCCTCCCGAGGTGGCTGTCTT | |
| | | CGAACCAAGCGAAGCCGAGATCTCTCACACACAAAAGGCTACT | |
| | | CTCGTGTGTCTGGCAACGGGTTTTTACCCTGATCATGTAGAGT | |
| | | TGTCTTGGTGGGTTAACGGCAAAGAAGTACACTCCGGTGTAAG | |
| | | CACCGATCCTCAGCCACTTAAGGAACAACCCGCACTCAACGAC | |
| | | TCCAGATACTGTTTGAGTTCTAGGCTGAGAGTCTCAGCGACGT | |
| | | TTTGGCAGAACCCACGGAATCATTTCAGATGTCAGGTCCAGTT | |
| | | TTACGGGTTGAGCGAGAACGACGAGTGGACACAGGATCGGGCT | |
| | | AAACCAGTGACCCAGATTGTATCAGCCGAAGCATGGGGAAGGG | |
| | | CAGATTGCGGCTTTACCTCAGAGTCATATCAACAGGGCGTTTT | |
| | | GTCTGCAACAATCCTTTATGAGATCCTGCTTGGTAAGGCGACT | |
| | | CTGTATGCCGTGCTTGTTAGTGCCCTGGTTCTGATGGCAATGG | |
| | | TAAAAAGAAAGGACTCTAGGGGAGGTTCTGGCGCTACTAACTT | |
| | | CAGCCTGCTGAAGCAGGCTGGAGACGTGGAGGAGAACCCTGGA | |
| | | CCTATGGCCATGCTCCTGGGGGCATCAGTGCTGATTCTGTGGC | |
| | | TTCAGCCAGACTGGGTAAACAGTCAACAGAAGAATGATGACCA | |
| | | GCAAGTTAAGCAAAATTCACCATCCCTGAGCGTCCAGGAAGGA | |
| | | AGAATTTCTATTCTGAACTGTGACTATACTAACAGCATGTTTG | |
| | | ATTATTTCCTATGGTACAAAAAATACCCTGCTGAAGGTCCTAC | |
| | | ATTCCTGATATCTATAAGTTCCATTAAGGATAAAAATGAAGAT | |
| | | GGAAGATTCACTGTCTTCTTAAACAAAAGTGCCAAGCACCTCT | |
| | | CTCTGCACATTGTGCCCTCCCAGCCTGGAGACTCTGCAGTGTA | |
| | | CTTCTGTGCAGCAACCCGTACCTCAGGAACCTACAAATACATC | |
| | | TTTGGAACAGGCACCAGGCTGAAGGTTTTAGCAAATATCCAGA | |
| | | ACCCTGACCCTGCAGTATATCAGCTGAGAGAC | |
| | Full-length | AACATACCATAAACCTCCCATTCTGCTAATGCCCAGCCTAAGT | 107 |
| | donor | TGGGGAGACCACTCCAGATTCCAAGATGTACAGTTTGCTTTGC | |
| | template | TGGGCCTTTTTCCCATGCCTGCCTTTACTCTGCCAGAGTTATA | |
| | | TTGCTGGGGTTTTGAAGAAGATCCTATTAAATAAAAGAATAAG | |
| | | CAGTATTATTAAGTAGCCCTGCATTTCAGGTTTCCTTGAGTGG | |
| | | CAGGCCAGGCCTGGCCGTGAACGTTCACTGAAATCATGGCCTC | |
| | | TTGGCCAAGATTGATAGCTTGTGCCTGTCCCTGAGTCCCAGTC | |
| | | CATCACGAGCAGCTGGTTTCTAAGATGCTATTTCCCGTATAAA | |
| | | GCATGAGACCGTGACTTGCCAGCCCCACAGAGCCCCGCCCTTG | |
| | | TCCATCACTGGCATCTGGACTCCAGCCTGGGTTGGGGCAAAGA | |
| | | GGGAAATGAGATCATGTCCTAACCCTGATCCTCTTGTCCCACA | |
| | | GATATCCAGAACCCTGACCCTGCCGTGTAGCAGCTTAGATACG | |
| | | AACAGAGAAACAGGAGAATATGGGCCAAACAGGATATCTGTGG | |
| | | TAAGCAGTTCCTGCCCCGGCTCAGGGCCAAGAACAGTTGGAAC | |
| | | AGCAGAATATGGGCCAAACAGGATATCTGTGGTAAGCAGTTCC | |
| | | TGCCCCGGCTCAGGGCCAAGAACAGATGGTCCCCAGATGCGGT | |
| | | CCCGCCCTCAGCAGTTTCTAGAGAACCATCAGATGTTTCCAGG | |
| | | GTGCCCCAAGGACCTGAAATGACCCTGTGCCTTATTTGAACTA | |
| | | ACCAATCAGTTCGCTTCTCGCTTCTGTTCGCGCGCTTCTGCTC | |
| | | CCCGAGCTCTATATAAGCAGAGCTCGTTTAGTGAACCGTCAGA | |
| | | TCGCCTGGAGACGCCATCCACGCTGTTTTGACTTCCATAGAAG | |
| | | GATCTCGCCGCCACCATGCCTCTGGGCCTGCTGTGGCTGGGCC | |
| | | TGGCCCTGCTGGGCGCCCTGCACGCCCAGGCCGGCGTGCAGGT | |
| | | GGAGACAATCTCCCCAGGCGACGGACGCACATTCCCTAAGCGG | |
| | | GGCCAGACCTGCGTGGTGCACTATACAGGCATGCTGGAGGATG | |
| | | GCAAGAAGTTTGACAGCTCCCGGGATAGAAACAAGCCATTCAA | |
| | | GTTTATGCTGGGCAAGCAGGAAGTGATCAGAGGCTGGGAGGAG | |
| | | GGCGTGGCCCAGATGTCTGTGGGCCAGAGGGCCAAGCTGACCA | |
| | | TCAGCCCAGACTACGCCTATGGAGCAACAGGCCACCCAGGAAT | |
| | | CATCCCACCTCACGCCACCCTGGTGTTCGATGTGGAGCTGCTG | |
| | | AAGCTGGGCGAGGGATCCAACACATCAAAAGAGAACCCCTTTC | |
| | | TGTTCGCATTGGAGGCCGTAGTCATATCTGTTGGATCCATGGG | |
| | | ACTTATTATCTCCCTGTTGTGTGTGTACTTCTGGCTGGAACGG | |
| | | ACTATGCCCAGGATCCCCACGCTCAAGAATCTGGAAGATCTCG | |
| | | TCACAGAATACCATGGTAATTTCAGCGCCTGGAGCGGAGTCTC | |
| | | TAAGGGTCTGGCCGAATCCCTCCAACCCGATTATTCTGAACGG | |
| | | TTGTGCCTCGTATCCGAAATACCACCAAAAGGCGGGGCTCTGG | |
| | | GTGAGGGCCCAGGGGCGAGTCCGTGCAATCAACACAGCCCGTA | |
| | | TTGGGCCCCTCCTTGTTATACGTTGAAGCCCGAAACTGGATCC | |
| | | GGCGAGGGCAGAGGAAGTCTGCTAACATGCGGTGACGTCGAGG | |
| | | AGAATCCTGGACCTATGAGCATCAGCCTCCTGTGCTGTGCAGC | |
| | | CTTTCCTCTCCTGTGGGCAGGTCCAGTGAATGCTGGTGTCACT | |
| | | CAGACCCCAAAATTCCGCATCCTGAAGATAGGACAGAGCATGA | |
| | | CACTGCAGTGTACCCAGGATATGAACCATAACTACATGTACTG | |
| | | GTATCGACAAGACCCAGGCATGGGGCTGAAGCTGATTTATTAT | |
| | | TCAGTTGGTGCTGGTATCACTGATAAAGGAGAAGTCCCGAATG | |
| | | GCTACAACGTCTCCAGATCAACCACAGAGGATTTCCCGCTCAG | |
| | | GCTGGAGTTGGCTGCTCCCTCCCAGACATCTGTGTACTTCTGT | |
| | | GCCAGCAGTCCGTGGGGGGCAGGCGGCACAGATACGCAGTATT | |
| | | TTGGCCCAGGCACCCGGCTGACAGTGCTCGAGGACCTTAAGAA | |
| | | TGTGTTCCCTCCCGAGGTGGCTGTCTTCGAACCAAGCGAAGCC | |
| | | GAGATCTCTCACACACAAAAGGCTACTCTCGTGTGTCTGGCAA | |
| | | CGGGTTTTTACCCTGATCATGTAGAGTTGTCTTGGTGGGTTAA | |
| | | CGGCAAAGAAGTACACTCCGGTGTAAGCACCGATCCTCAGCCA | |
| | | CTTAAGGAACAACCCGCACTCAACGACTCCAGATACTGTTTGA | |
| | | GTTCTAGGCTGAGAGTCTCAGCGACGTTTTGGCAGAACCCACG | |
| | | GAATCATTTCAGATGTCAGGTCCAGTTTTACGGGTTGAGCGAG | |
| | | AACGACGAGTGGACACAGGATCGGGCTAAACCAGTGACCCAGA | |
| | | TTGTATCAGCCGAAGCATGGGGAAGGGCAGATTGCGGCTTTAC | |
| | | CTCAGAGTCATATCAACAGGGCGTTTTGTCTGCAACAATCCTT | |
| | | TATGAGATCCTGCTTGGTAAGGCGACTCTGTATGCCGTGCTTG | |
| | | TTAGTGCCCTGGTTCTGATGGCAATGGTAAAAAGAAAGGACTC | |
| | | TAGGGGAGGTTCTGGCGCTACTAACTTCAGCCTGCTGAAGCAG | |
| | | GCTGGAGACGTGGAGGAGAACCCTGGACCTATGGCCATGCTCC | |
| | | TGGGGGCATCAGTGCTGATTCTGTGGCTTCAGCCAGACTGGGT | |
| | | AAACAGTCAACAGAAGAATGATGACCAGCAAGTTAAGCAAAAT | |
| | | TCACCATCCCTGAGCGTCCAGGAAGGAAGAATTTCTATTCTGA | |
| | | ACTGTGACTATACTAACAGCATGTTTGATTATTTCCTATGGTA | |
| | | CAAAAAATACCCTGCTGAAGGTCCTACATTCCTGATATCTATA | |
| | | AGTTCCATTAAGGATAAAAATGAAGATGGAAGATTCACTGTCT | |
| | | TCTTAAACAAAAGTGCCAAGCACCTCTCTCTGCACATTGTGCC | |
| | | CTCCCAGCCTGGAGACTCTGCAGTGTACTTCTGTGCAGCAACC | |
| | | CGTACCTCAGGAACCTACAAATACATCTTTGGAACAGGCACCA | |
| | | GGCTGAAGGTTTTAGCAAATATCCAGAACCCTGACCCTGCAGT | |
| | | ATATCAGCTGAGAGACTCTAAATCCAGTGACAAGTCTGTCTGC | |
| | | CTATTCACCGATTTTGATTCTCAAACAAATGTGTCACAAAGTA | |
| | | AGGATTCTGATGTGTATATCACAGACAAAACTGTGCTAGACAT | |
| | | GAGGTCTATGGACTTCAAGAGCAACAGTGCTGTGGCCTGGAGC | |
| | | AACAAATCTGACTTTGCATGTGCAAACGCCTTCAACAACAGCA | |
| | | TTATTCCAGAAGACACCTTCTTCCCCAGCCCAGGTAAGGGCAG | |
| | | CTTTGGTGCCTTCGCAGGCTGTTTCCTTGCTTCAGGAATGGCC | |
| | | AGGTTCTGCCCAGAGCTCTGGTCAATGATGTCTAAAACTCCTC | |
| | | TGATTGGTGGTCTCGGCCTTATCCATTGCCACCAAAACCCTCT | |
| | | TTTTACTAAGAAACAGTGAGCCTTGTTCTGGCAGTCCAGAGAA | |
| | | TGACACGGGAAAAAAGCAGATGAAGAGAAGGTGGCAGGAGAGG | |
| | | GCACGTGGCCCAGCCTCAGTCTCTCCAACTGAGTTCCTGCCTG | |
| TID2_v3 | 5′ Homology | AGCAATCTCCTGGTAATGTGATAGATTTCCCAACTTAATGCCA | 108 |
| | Arm | ACATACCATAAACCTCCCATTCTGCTAATGCCCAGCCTAAGTT | |
| | | GGGGAGACCACTCCAGATTCCAAGATGTACAGTTTGCTTTGCT | |
| | | GGGCCTTTTTCCCATGCCTGCCTTTACTCTGCCAGAGTTATAT | |
| | | TGCTGGGGTTTTGAAGAAGATCCTATTAAATAAAAGAATAAGC | |
| | | AGTATTATTAAGTAGCCCTGCATTTCAGGTTTCCTTGAGTGGC | |
| | | AGGCCAGGCCTGGCCGTGAACGTTCACTGAAATCATGGCCTCT | |
| | | TGGCCAAGATTGATAGCTTGTGCCTGTCCCTGAGTCCCAGTCC | |
| | | ATCACGAGCAGCTGGTTTCTAAGATGCTATTTCCCGTATAAAG | |
| | | CATGAGACCGTGACTTGCCAGCCCCACAGAGCCCCGCCCTTGT | |
| | | CCATCACTGGCATCTGGACTCCAGCCTGGGTTGGGGCAAAGAG | |
| | | GGAAATGAGATCATGTCCTAACCCTGA | |
| | Intervening | TA | |
| | sequence | | |
| | MND | GAACAGAGAAACAGGAGAATATGGGCCAAACAGGATATCTGTG | 109 |
| | Promoter | GTAAGCAGTTCCTGCCCCGGCTCAGGGCCAAGAACAGTTGGAA | |
| | | CAGCAGAATATGGGCCAAACAGGATATCTGTGGTAAGCAGTTC | |
| | | CTGCCCCGGCTCAGGGCCAAGAACAGATGGTCCCCAGATGCGG | |
| | | TCCCGCCCTCAGCAGTTTCTAGAGAACCATCAGATGTTTCCAG | |
| | | GGTGCCCCAAGGACCTGAAATGACCCTGTGCCTTATTTGAACT | |
| | | AACCAATCAGTTCGCTTCTCGCTTCTGTTCGCGCGCTTCTGCT | |
| | | CCCCGAGCTCTATATAAGCAGAGCTCGTTTAGTGAACCGTCAG | |
| | | ATC | |
| | Intervening | GCCTGGAGACGCCATCCACGCTGTTTTGACTTCCATAGAAGGA | 110 |
| | Sequence | TCTCGCCGCCACCATGCCTCTGGGCCTGCTGTGGCTGGGCCTG | |
| | (LCN2 | GCCCTGCTGGGCGCCCTGCACGCCCAGGCC | |
| | signal | | |
| | peptide of | | |
| | FKBP-IL2Rg | | |
| | underlined) | | |
| | FKBP-IL2Rg | GGCGTGCAGGTGGAGACAATCTCCCCAGGCGACGGACGCACAT | 111 |
| | | TCCCTAAGCGGGGCCAGACCTGCGTGGTGCACTATACAGGCAT | |
| | | GCTGGAGGATGGCAAGAAGTTTGACAGCTCCCGGGATAGAAAC | |
| | | AAGCCATTCAAGTTTATGCTGGGCAAGCAGGAAGTGATCAGAG | |
| | | GCTGGGAGGAGGGCGTGGCCCAGATGTCTGTGGGCCAGAGGGC | |
| | | CAAGCTGACCATCAGCCCAGACTACGCCTATGGAGCAACAGGC | |
| | | CACCCAGGAATCATCCCACCTCACGCCACCCTGGTGTTCGATG | |
| | | TGGAGCTGCTGAAGCTGGGCGAGGGATCCAACACATCAAAAGA | |
| | | GAACCCCTTTCTGTTCGCATTGGAGGCCGTAGTCATATCTGTT | |
| | | GGATCCATGGGACTTATTATCTCCCTGTTGTGTGTGTACTTCT | |
| | | GGCTGGAACGGACTATGCCCAGGATCCCCACGCTCAAGAATCT | |
| | | GGAAGATCTCGTCACAGAATACCATGGTAATTTCAGCGCCTGG | |
| | | AGCGGAGTCTCTAAGGGTCTGGCCGAATCCCTCCAACCCGATT | |
| | | ATTCTGAACGGTTGTGCCTCGTATCCGAAATACCACCAAAAGG | |
| | | CGGGGCTCTGGGTGAGGGCCCAGGGGCGAGTCCGTGCAATCAA | |
| | | CACAGCCCGTATTGGGCCCCTCCTTGTTATACGTTGAAGCCCG | |
| | | AAACT | |
| | GSG linker | GGATCCGGC | |
| | T2A | GAGGGCAGAGGAAGTCTGCTAACATGCGGTGACGTCGAGGAGA | 112 |
| | T1D2 TCRβ | ATCCTGGACCT | 113 |
| | | ATGAGCATCAGCCTCCTGTGCTGTGCAGCCTTTCCTCTCCTGT | |
| | | GGGCAGGTCCAGTGAATGCTGGTGTCACTCAGACCCCAAAATT | |
| | | CCGCATCCTGAAGATAGGACAGAGCATGACACTGCAGTGTACC | |
| | | CAGGATATGAACCATAACTACATGTACTGGTATCGACAAGACC | |
| | | CAGGCATGGGGCTGAAGCTGATTTATTATTCAGTTGGTGCTGG | |
| | | TATCACTGATAAAGGAGAAGTCCCGAATGGCTACAACGTCTCC | |
| | | AGATCAACCACAGAGGATTTCCCGCTCAGGCTGGAGTTGGCTG | |
| | | CTCCCTCCCAGACATCTGTGTACTTCTGTGCCAGCAGTCCGTG | |
| | | GGGGGCAGGCGGCACAGATACGCAGTATTTTGGCCCAGGCACC | |
| | | CGGCTGACAGTGCTCGAGGACCTTAAGAATGTGTTCCCTCCCG | |
| | | AGGTGGCTGTCTTCGAACCAAGCGAAGCCGAGATCTCTCACAC | |
| | | ACAAAAGGCTACTCTCGTGTGTCTGGCAACGGGTTTTTACCCT | |
| | | GATCATGTAGAGTTGTCTTGGTGGGTTAACGGCAAAGAAGTAC | |
| | | ACTCCGGTGTAAGCACCGATCCTCAGCCACTTAAGGAACAACC | |
| | | CGCACTCAACGACTCCAGATACTGTTTGAGTTCTAGGCTGAGA | |
| | | GTCTCAGCGACGTTTTGGCAGAACCCACGGAATCATTTCAGAT | |
| | | GTCAGGTCCAGTTTTACGGGTTGAGCGAGAACGACGAGTGGAC | |
| | | ACAGGATCGGGCTAAACCAGTGACCCAGATTGTATCAGCCGAA | |
| | | GCATGGGGAAGGGCAGATTGCGGCTTTACCTCAGAGTCATATC | |
| | | AACAGGGCGTTTTGTCTGCAACAATCCTTTATGAGATCCTGCT | |
| | | TGGTAAGGCGACTCTGTATGCCGTGCTTGTTAGTGCCCTGGTT | |
| | | CTGATGGCAATGGTAAAAAGAAAGGACTCTAGGGGA | |
| | GSG linker | GGTTCTGGC | |
| | P2A | GCTACTAACTTCAGCCTGCTGAAGCAGGCTGGAGACGTGGAGG | 114 |
| | | AGAACCCTGGACCT | |
| | T1D2 TCRα | ATGGCCATGCTCCTGGGGGCATCAGTGCTGATTCTGTGGCTTC | 115 |
| | Vα domain | AGCCAGACTGGGTAAACAGTCAACAGAAGAATGATGACCAGCA | |
| | | AGTTAAGCAAAATTCACCATCCCTGAGCGTCCAGGAAGGAAGA | |
| | | ATTTCTATTCTGAACTGTGACTATACTAACAGCATGTTTGATT | |
| | | ATTTCCTATGGTACAAAAAATACCCTGCTGAAGGTCCTACATT | |
| | | CCTGATATCTATAAGTTCCATTAAGGATAAAAATGAAGATGGA | |
| | | AGATTCACTGTCTTCTTAAACAAAAGTGCCAAGCACCTCTCTC | |
| | | TGCACATTGTGCCCTCCCAGCCTGGAGACTCTGCAGTGTACTT | |
| | | CTGTGCAGCAACCCGTACCTCAGGAACCTACAAATACATCTTT | |
| | | GGAACAGGCACCAGGCTGAAGGTTTTAGCAA | |
| | 3′ Homology | ATATCCAGAACCCTGACCCTGCCGTGTACCAGCTGAGAGACTC | 116 |
| | Arm | TAAATCCAGTGACAAGTCTGTCTGCCTATTCACCGATTTTGAT | |
| | | TCTCAAACAAATGTGTCACAAAGTAAGGATTCTGATGTGTATA | |
| | | TCACAGACAAAACTGTGCTAGACATGAGGTCTATGGACTTCAA | |
| | | GAGCAACAGTGCTGTGGCCTGGAGCAACAAATCTGACTTTGCA | |
| | | TGTGCAAACGCCTTCAACAACAGCATTATTCCAGAAGACACCT | |
| | | TCTTCCCCAGCCCAGGTAAGGGCAGCTTTGGTGCCTTCGCAGG | |
| | | CTGTTTCCTTGCTTCAGGAATGGCCAGGTTCTGCCCAGAGCTC | |
| | | TGGTCAATGATGTCTAAAACTCCTCTGATTGGTGGTCTCGGCC | |
| | | TTATCCATTGCCACCAAAACCCTCTTTTTACTAAGAAACAGTG | |
| | | AGCCTTGTTCTGGCAGTCCAGAGAATGACACGGGAAAAAAGCA | |
| | | GATGAAGAGAAGGTGGCAGGAGAGGGC | |
| | Between | TAGAACAGAGAAACAGGAGAATATGGGCCAAACAGGATATCTG | 117 |
| | Homology | TGGTAAGCAGTTCCTGCCCCGGCTCAGGGCCAAGAACAGTTGG | |
| | Arms | AACAGCAGAATATGGGCCAAACAGGATATCTGTGGTAAGCAGT | |
| | | TCCTGCCCCGGCTCAGGGCCAAGAACAGATGGTCCCCAGATGC | |
| | | GGTCCCGCCCTCAGCAGTTTCTAGAGAACCATCAGATGTTTCC | |
| | | AGGGTGCCCCAAGGACCTGAAATGACCCTGTGCCTTATTTGAA | |
| | | CTAACCAATCAGTTCGCTTCTCGCTTCTGTTCGCGCGCTTCTG | |
| | | CTCCCCGAGCTCTATATAAGCAGAGCTCGTTTAGTGAACCGTC | |
| | | AGATCGCCTGGAGACGCCATCCACGCTGTTTTGACTTCCATAG | |
| | | AAGGATCTCGCCGCCACCATGCCTCTGGGCCTGCTGTGGCTGG | |
| | | GCCTGGCCCTGCTGGGCGCCCTGCACGCCCAGGCCGGCGTGCA | |
| | | GGTGGAGACAATCTCCCCAGGCGACGGACGCACATTCCCTAAG | |
| | | CGGGGCCAGACCTGCGTGGTGCACTATACAGGCATGCTGGAGG | |
| | | ATGGCAAGAAGTTTGACAGCTCCCGGGATAGAAACAAGCCATT | |
| | | CAAGTTTATGCTGGGCAAGCAGGAAGTGATCAGAGGCTGGGAG | |
| | | GAGGGCGTGGCCCAGATGTCTGTGGGCCAGAGGGCCAAGCTGA | |
| | | CCATCAGCCCAGACTACGCCTATGGAGCAACAGGCCACCCAGG | |
| | | AATCATCCCACCTCACGCCACCCTGGTGTTCGATGTGGAGCTG | |
| | | CTGAAGCTGGGCGAGGGATCCAACACATCAAAAGAGAACCCCT | |
| | | TTCTGTTCGCATTGGAGGCCGTAGTCATATCTGTTGGATCCAT | |
| | | GGGACTTATTATCTCCCTGTTGTGTGTGTACTTCTGGCTGGAA | |
| | | CGGACTATGCCCAGGATCCCCACGCTCAAGAATCTGGAAGATC | |
| | | TCGTCACAGAATACCATGGTAATTTCAGCGCCTGGAGCGGAGT | |
| | | CTCTAAGGGTCTGGCCGAATCCCTCCAACCCGATTATTCTGAA | |
| | | CGGTTGTGCCTCGTATCCGAAATACCACCAAAAGGCGGGGCTC | |
| | | TGGGTGAGGGCCCAGGGGCGAGTCCGTGCAATCAACACAGCCC | |
| | | GTATTGGGCCCCTCCTTGTTATACGTTGAAGCCCGAAACTGGA | |
| | | TCCGGCGAGGGCAGAGGAAGTCTGCTAACATGCGGTGACGTCG | |
| | | AGGAGAATCCTGGACCTATGAGCATCAGCCTCCTGTGCTGTGC | |
| | | AGCCTTTCCTCTCCTGTGGGCAGGTCCAGTGAATGCTGGTGTC | |
| | | ACTCAGACCCCAAAATTCCGCATCCTGAAGATAGGACAGAGCA | |
| | | TGACACTGCAGTGTACCCAGGATATGAACCATAACTACATGTA | |
| | | CTGGTATCGACAAGACCCAGGCATGGGGCTGAAGCTGATTTAT | |
| | | TATTCAGTTGGTGCTGGTATCACTGATAAAGGAGAAGTCCCGA | |
| | | ATGGCTACAACGTCTCCAGATCAACCACAGAGGATTTCCCGCT | |
| | | CAGGCTGGAGTTGGCTGCTCCCTCCCAGACATCTGTGTACTTC | |
| | | TGTGCCAGCAGTCCGTGGGGGGCAGGCGGCACAGATACGCAGT | |
| | | ATTTTGGCCCAGGCACCCGGCTGACAGTGCTCGAGGACCTTAA | |
| | | GAATGTGTTCCCTCCCGAGGTGGCTGTCTTCGAACCAAGCGAA | |
| | | GCCGAGATCTCTCACACACAAAAGGCTACTCTCGTGTGTCTGG | |
| | | CAACGGGTTTTTACCCTGATCATGTAGAGTTGTCTTGGTGGGT | |
| | | TAACGGCAAAGAAGTACACTCCGGTGTAAGCACCGATCCTCAG | |
| | | CCACTTAAGGAACAACCCGCACTCAACGACTCCAGATACTGTT | |
| | | TGAGTTCTAGGCTGAGAGTCTCAGCGACGTTTTGGCAGAACCC | |
| | | ACGGAATCATTTCAGATGTCAGGTCCAGTTTTACGGGTTGAGC | |
| | | GAGAACGACGAGTGGACACAGGATCGGGCTAAACCAGTGACCC | |
| | | AGATTGTATCAGCCGAAGCATGGGGAAGGGCAGATTGCGGCTT | |
| | | TACCTCAGAGTCATATCAACAGGGCGTTTTGTCTGCAACAATC | |
| | | CTTTATGAGATCCTGCTTGGTAAGGCGACTCTGTATGCCGTGC | |
| | | TTGTTAGTGCCCTGGTTCTGATGGCAATGGTAAAAAGAAAGGA | |
| | | CTCTAGGGGAGGTTCTGGCGCTACTAACTTCAGCCTGCTGAAG | |
| | | CAGGCTGGAGACGTGGAGGAGAACCCTGGACCTATGGCCATGC | |
| | | TCCTGGGGGCATCAGTGCTGATTCTGTGGCTTCAGCCAGACTG | |
| | | GGTAAACAGTCAACAGAAGAATGATGACCAGCAAGTTAAGCAA | |
| | | AATTCACCATCCCTGAGCGTCCAGGAAGGAAGAATTTCTATTC | |
| | | TGAACTGTGACTATACTAACAGCATGTTTGATTATTTCCTATG | |
| | | GTACAAAAAATACCCTGCTGAAGGTCCTACATTCCTGATATCT | |
| | | ATAAGTTCCATTAAGGATAAAAATGAAGATGGAAGATTCACTG | |
| | | TCTTCTTAAACAAAAGTGCCAAGCACCTCTCTCTGCACATTGT | |
| | | GCCCTCCCAGCCTGGAGACTCTGCAGTGTACTTCTGTGCAGCA | |
| | | ACCCGTACCTCAGGAACCTACAAATACATCTTTGGAACAGGCA | |
| | | CCAGGCTGAAGGTTTTAGCAA | |
| | Full-length | AGCAATCTCCTGGTAATGTGATAGATTTCCCAACTTAATGCCA | 118 |
| | donor | ACATACCATAAACCTCCCATTCTGCTAATGCCCAGCCTAAGTT | |
| | template | GGGGAGACCACTCCAGATTCCAAGATGTACAGTTTGCTTTGCT | |
| | | GGGCCTTTTTCCCATGCCTGCCTTTACTCTGCCAGAGTTATAT | |
| | | TGCTGGGGTTTTGAAGAAGATCCTATTAAATAAAAGAATAAGC | |
| | | AGTATTATTAAGTAGCCCTGCATTTCAGGTTTCCTTGAGTGGC | |
| | | AGGCCAGGCCTGGCCGTGAACGTTCACTGAAATCATGGCCTCT | |
| | | TGGCCAAGATTGATAGCTTGTGCCTGTCCCTGAGTCCCAGTCC | |
| | | ATCACGAGCAGCTGGTTTCTAAGATGCTATTTCCCGTATAAAG | |
| | | CATGAGACCGTGACTTGCCAGCCCCACAGAGCCCCGCCCTTGT | |
| | | CCATCACTGGCATCTGGACTCCAGCCTGGGTTGGGGCAAAGAG | |
| | | GGAAATGAGATCATGTCCTAACCCTGATAGAACAGAGAAACAG | |
| | | GAGAATATGGGCCAAACAGGATATCTGTGGTAAGCAGTTCCTG | |
| | | CCCCGGCTCAGGGCCAAGAACAGTTGGAACAGCAGAATATGGG | |
| | | CCAAACAGGATATCTGTGGTAAGCAGTTCCTGCCCCGGCTCAG | |
| | | GGCCAAGAACAGATGGTCCCCAGATGCGGTCCCGCCCTCAGCA | |
| | | GTTTCTAGAGAACCATCAGATGTTTCCAGGGTGCCCCAAGGAC | |
| | | CTGAAATGACCCTGTGCCTTATTTGAACTAACCAATCAGTTCG | |
| | | CTTCTCGCTTCTGTTCGCGCGCTTCTGCTCCCCGAGCTCTATA | |
| | | TAAGCAGAGCTCGTTTAGTGAACCGTCAGATCGCCTGGAGACG | |
| | | CCATCCACGCTGTTTTGACTTCCATAGAAGGATCTCGCCGCCA | |
| | | CCATGCCTCTGGGCCTGCTGTGGCTGGGCCTGGCCCTGCTGGG | |
| | | CGCCCTGCACGCCCAGGCCGGCGTGCAGGTGGAGACAATCTCC | |
| | | CCAGGCGACGGACGCACATTCCCTAAGCGGGGCCAGACCTGCG | |
| | | TGGTGCACTATACAGGCATGCTGGAGGATGGCAAGAAGTTTGA | |
| | | CAGCTCCCGGGATAGAAACAAGCCATTCAAGTTTATGCTGGGC | |
| | | AAGCAGGAAGTGATCAGAGGCTGGGAGGAGGGCGTGGCCCAGA | |
| | | TGTCTGTGGGCCAGAGGGCCAAGCTGACCATCAGCCCAGACTA | |
| | | CGCCTATGGAGCAACAGGCCACCCAGGAATCATCCCACCTCAC | |
| | | GCCACCCTGGTGTTCGATGTGGAGCTGCTGAAGCTGGGCGAGG | |
| | | GATCCAACACATCAAAAGAGAACCCCTTTCTGTTCGCATTGGA | |
| | | GGCCGTAGTCATATCTGTTGGATCCATGGGACTTATTATCTCC | |
| | | CTGTTGTGTGTGTACTTCTGGCTGGAACGGACTATGCCCAGGA | |
| | | TCCCCACGCTCAAGAATCTGGAAGATCTCGTCACAGAATACCA | |
| | | TGGTAATTTCAGCGCCTGGAGCGGAGTCTCTAAGGGTCTGGCC | |
| | | GAATCCCTCCAACCCGATTATTCTGAACGGTTGTGCCTCGTAT | |
| | | CCGAAATACCACCAAAAGGCGGGGCTCTGGGTGAGGGCCCAGG | |
| | | GGCGAGTCCGTGCAATCAACACAGCCCGTATTGGGCCCCTCCT | |
| | | TGTTATACGTTGAAGCCCGAAACTGGATCCGGCGAGGGCAGAG | |
| | | GAAGTCTGCTAACATGCGGTGACGTCGAGGAGAATCCTGGACC | |
| | | TATGAGCATCAGCCTCCTGTGCTGTGCAGCCTTTCCTCTCCTG | |
| | | TGGGCAGGTCCAGTGAATGCTGGTGTCACTCAGACCCCAAAAT | |
| | | TCCGCATCCTGAAGATAGGACAGAGCATGACACTGCAGTGTAC | |
| | | CCAGGATATGAACCATAACTACATGTACTGGTATCGACAAGAC | |
| | | CCAGGCATGGGGCTGAAGCTGATTTATTATTCAGTTGGTGCTG | |
| | | GTATCACTGATAAAGGAGAAGTCCCGAATGGCTACAACGTCTC | |
| | | CAGATCAACCACAGAGGATTTCCCGCTCAGGCTGGAGTTGGCT | |
| | | GCTCCCTCCCAGACATCTGTGTACTTCTGTGCCAGCAGTCCGT | |
| | | GGGGGGCAGGCGGCACAGATACGCAGTATTTTGGCCCAGGCAC | |
| | | CCGGCTGACAGTGCTCGAGGACCTTAAGAATGTGTTCCCTCCC | |
| | | GAGGTGGCTGTCTTCGAACCAAGCGAAGCCGAGATCTCTCACA | |
| | | CACAAAAGGCTACTCTCGTGTGTCTGGCAACGGGTTTTTACCC | |
| | | TGATCATGTAGAGTTGTCTTGGTGGGTTAACGGCAAAGAAGTA | |
| | | CACTCCGGTGTAAGCACCGATCCTCAGCCACTTAAGGAACAAC | |
| | | CCGCACTCAACGACTCCAGATACTGTTTGAGTTCTAGGCTGAG | |
| | | AGTCTCAGCGACGTTTTGGCAGAACCCACGGAATCATTTCAGA | |
| | | TGTCAGGTCCAGTTTTACGGGTTGAGCGAGAACGACGAGTGGA | |
| | | CACAGGATCGGGCTAAACCAGTGACCCAGATTGTATCAGCCGA | |
| | | AGCATGGGGAAGGGCAGATTGCGGCTTTACCTCAGAGTCATAT | |
| | | CAACAGGGCGTTTTGTCTGCAACAATCCTTTATGAGATCCTGC | |
| | | TTGGTAAGGCGACTCTGTATGCCGTGCTTGTTAGTGCCCTGGT | |
| | | TCTGATGGCAATGGTAAAAAGAAAGGACTCTAGGGGAGGTTCT | |
| | | GGCGCTACTAACTTCAGCCTGCTGAAGCAGGCTGGAGACGTGG | |
| | | AGGAGAACCCTGGACCTATGGCCATGCTCCTGGGGGCATCAGT | |
| | | GCTGATTCTGTGGCTTCAGCCAGACTGGGTAAACAGTCAACAG | |
| | | AAGAATGATGACCAGCAAGTTAAGCAAAATTCACCATCCCTGA | |
| | | GCGTCCAGGAAGGAAGAATTTCTATTCTGAACTGTGACTATAC | |
| | | TAACAGCATGTTTGATTATTTCCTATGGTACAAAAAATACCCT | |
| | | GCTGAAGGTCCTACATTCCTGATATCTATAAGTTCCATTAAGG | |
| | | ATAAAAATGAAGATGGAAGATTCACTGTCTTCTTAAACAAAAG | |
| | | TGCCAAGCACCTCTCTCTGCACATTGTGCCCTCCCAGCCTGGA | |
| | | GACTCTGCAGTGTACTTCTGTGCAGCAACCCGTACCTCAGGAA | |
| | | CCTACAAATACATCTTTGGAACAGGCACCAGGCTGAAGGTTTT | |
| | | AGCAAATATCCAGAACCCTGACCCTGCCGTGTACCAGCTGAGA | |
| | | GACTCTAAATCCAGTGACAAGTCTGTCTGCCTATTCACCGATT | |
| | | TTGATTCTCAAACAAATGTGTCACAAAGTAAGGATTCTGATGT | |
| | | GTATATCACAGACAAAACTGTGCTAGACATGAGGTCTATGGAC | |
| | | TTCAAGAGCAACAGTGCTGTGGCCTGGAGCAACAAATCTGACT | |
| | | TTGCATGTGCAAACGCCTTCAACAACAGCATTATTCCAGAAGA | |
| | | CACCTTCTTCCCCAGCCCAGGTAAGGGCAGCTTTGGTGCCTTC | |
| | | GCAGGCTGTTTCCTTGCTTCAGGAATGGCCAGGTTCTGCCCAG | |
| | | AGCTCTGGTCAATGATGTCTAAAACTCCTCTGATTGGTGGTCT | |
| | | CGGCCTTATCCATTGCCACCAAAACCCTCTTTTTACTAAGAAA | |
| | | CAGTGAGCCTTGTTCTGGCAGTCCAGAGAATGACACGGGAAAA | |
| | | AAGCAGATGAAGAGAAGGTGGCAGGAGAGGGC | |
| T1D4 | 5′ Homology | CCTATTAAATAAAAGAATAAGCAGTATTATTAAGTAGCCCTGC | 119 |
| | Arm | ATTTCAGGTTTCCTTGAGTGGCAGGCCAGGCCTGGCCGTGAAC | |
| | | GTTCACTGAAATCATGGCCTCTTGGCCAAGATTGATAGCTTGT | |
| | | GCCTGTCCCTGAGTCCCAGTCCATCACGAGCAGCTGGTTTCTA | |
| | | AGATGCTATTTCCCGTATAAAGCATGAGACCGTGACTTGCCAG | |
| | | CCCCACAGAGCCCCGCCCTTGTCCATCACTGGCATCTGGACTC | |
| | | CAGCCTGGGTTGGGGCAAAGAGGGAAATGAGATCATGTCCTAA | |
| | | CCCTGATCCTCTTGTCCCACAGATATCCAGAACCCTGACCCTG | |
| | | CC | |
| | Intervening | TA | |
| | sequence | | |
| | MND | GAACAGAGAAACAGGAGAATATGGGCCAAACAGGATATCTGTG | 120 |
| | Promoter | GTAAGCAGTTCCTGCCCCGGCTCAGGGCCAAGAACAGTTGGAA | |
| | | CAGCAGAATATGGGCCAAACAGGATATCTGTGGTAAGCAGTTC | |
| | | CTGCCCCGGCTCAGGGCCAAGAACAGATGGTCCCCAGATGCGG | |
| | | TCCCGCCCTCAGCAGTTTCTAGAGAACCATCAGATGTTTCCAG | |
| | | GGTGCCCCAAGGACCTGAAATGACCCTGTGCCTTATTTGAACT | |
| | | AACCAATCAGTTCGCTTCTCGCTTCTGTTCGCGCGCTTCTGCT | |
| | | CCCCGAGCTCTATATAAGCAGAGCTCGTTTAGTGAACCGTCAG | |
| | | ATC | |
| | Intervening | GCCTGGAGACGCCATCCACGCTGTTTTGACTTCCATAGAAGGA | 121 |
| | Sequence | TCTCGCCGCCACCATGCCTCTGGGCCTGCTGTGGCTGGGCCTG | |
| | (LCN2 | GCCCTGCTGGGCGCCCTGCACGCCCAGGCC | |
| | signal | | |
| | peptide of | | |
| | FKBP-IL2Rg | | |
| | underlined | | |
| | FKBP-IL2Rg | GGCGTGCAGGTGGAGACAATCTCCCCAGGCGACGGACGCACAT | 122 |
| | | TCCCTAAGCGGGGCCAGACCTGCGTGGTGCACTATACAGGCAT | |
| | | GCTGGAGGATGGCAAGAAGTTTGACAGCTCCCGGGATAGAAAC | |
| | | AAGCCATTCAAGTTTATGCTGGGCAAGCAGGAAGTGATCAGAG | |
| | | GCTGGGAGGAGGGCGTGGCCCAGATGTCTGTGGGCCAGAGGGC | |
| | | CAAGCTGACCATCAGCCCAGACTACGCCTATGGAGCAACAGGC | |
| | | CACCCAGGAATCATCCCACCTCACGCCACCCTGGTGTTCGATG | |
| | | TGGAGCTGCTGAAGCTGGGCGAGGGATCCAACACATCAAAAGA | |
| | | GAACCCCTTTCTGTTCGCATTGGAGGCCGTAGTCATATCTGTT | |
| | | GGATCCATGGGACTTATTATCTCCCTGTTGTGTGTGTACTTCT | |
| | | GGCTGGAACGGACTATGCCCAGGATCCCCACGCTCAAGAATCT | |
| | | GGAAGATCTCGTCACAGAATACCATGGTAATTTCAGCGCCTGG | |
| | | AGCGGAGTCTCTAAGGGTCTGGCCGAATCCCTCCAACCCGATT | |
| | | ATTCTGAACGGTTGTGCCTCGTATCCGAAATACCACCAAAAGG | |
| | | CGGGGCTCTGGGTGAGGGCCCAGGGGCGAGTCCGTGCAATCAA | |
| | | CACAGCCCGTATTGGGCCCCTCCTTGTTATACGTTGAAGCCCG | |
| | | AAACT | |
| | GSG linker | GGATCCGGC | |
| | T2A | GAGGGCAGAGGAAGTCTGCTAACATGCGGTGACGTCGAGGAGA | 123 |
| | | ATCCTGGACCT | |
| | T1D4 TCRβ | ATGGGATCAAGACTGCTGTGCTGGGTGCTGCTGTGCCTGCTGG | 12 |
| | | GAGCCGGACCTGTGAAAGCCGGGGTGACTCAGACTCCACGATA | |
| | | CCTGATCAAGACCAGGGGCCAGCAGGTGACACTGTCTTGCAGC | |
| | | CCAATCTCCGGACACCGCTCCGTGTCTTGGTACCAGCAGACCC | |
| | | CTGGACAGGGACTGCAGTTCCTGTTTGAGTATTTCTCTGAGAC | |
| | | ACAGCGGAACAAGGGCAATTTCCCCGGCCGGTTTAGCGGCAGA | |
| | | CAGTTTAGCAACTCCAGGTCTGAGATGAATGTGAGCACCCTGG | |
| | | AGCTGGGCGACTCCGCCCTGTACCTGTGCGCAAGCTCCCTGGC | |
| | | CCTGGGACAGGGGAATCAGCAGTTTTTCGGACCCGGAACAAGG | |
| | | CTGACCGTGCTGGAGGACCTTAAGAATGTGTTCCCTCCCGAGG | |
| | | TGGCTGTCTTCGAACCAAGCGAAGCCGAGATCTCTCACACACA | |
| | | AAAGGCTACTCTCGTGTGTCTGGCAACGGGTTTTTACCCTGAT | |
| | | CATGTAGAGTTGTCTTGGTGGGTTAACGGCAAAGAAGTACACT | |
| | | CCGGTGTATCTACCGATCCTCAGCCACTTAAGGAACAACCCGC | |
| | | ACTCAACGACTCCAGATACTGTTTGAGTTCTAGGCTGAGAGTC | |
| | | TCAGCGACGTTTTGGCAGAACCCACGGAATCATTTCAGATGTC | |
| | | AGGTCCAGTTTTACGGGTTGAGCGAGAACGACGAGTGGACACA | |
| | | GGATCGGGCTAAACCAGTGACCCAGATTGTATCAGCCGAAGCA | |
| | | TGGGGAAGGGCAGATTGCGGCTTTACCTCAGAGTCATATCAAC | |
| | | AGGGCGTTTTGTCTGCAACAATCCTTTATGAGATCCTGCTTGG | |
| | | TAAGGCGACTCTGTATGCCGTGCTTGTTAGTGCCCTGGTTCTG | |
| | | ATGGCAATGGTAAAAAGAAAGGACTCTAGGGGA | |
| | GSG linker | GGTTCTGGC | |
| | P2A | GCTACTAACTTCAGCCTGCTGAAGCAGGCTGGAGACGTGGAGG | 125 |
| | | AGAACCCTGGACCT | |
| | T1D4 TCRα | ATGAAGAGCCTGCGCGTGCTGCTGGTCATCCTGTGGCTGCAAT | 126 |
| | Vα domain | TGAGTTGGGTGTGGAGCCAGAAGGACCAGGTGTTTCAGCCTAG | |
| | and C | CACTGTCGCATCATCAGAGGGGGCCGTCGTGGAAATCTTTTGT | |
| | domain | AATCACAGCGTGTCCAACGCCTACAATTTCTTTTGGTATCTGC | |
| | portion | ACTTCCCAGGATGCGCACCTAGGCTGCTGGTGAAGGGCTCTAA | |
| | | GCCCAGCCAGCAGGGCCGGTACAACATGACCTATGAGCGGTTC | |
| | | AGCTCCTCTCTGCTGATCCTGCAGGTGCGGGAGGCAGACGCAG | |
| | | CCGTGTACTATTGTGCCGTGGAGGATCTGAATCAGGCCGGCAC | |
| | | AGCCCTGATTTTCGGCAAAGGAACAACCCTGAGCGTGAGCAGC | |
| | | AATATCCAGAACCCTGACCCTGCAGTATAT | |
| | 3′ Homology | CAGCTGAGAGACTCTAAATCCAGTGACAAGTCTGTCTGCCTAT | 127 |
| | Arm | TCACCGATTTTGATTCTCAAACAAATGTGTCACAAAGTAAGGA | |
| | | TTCTGATGTGTATATCACAGACAAAACTGTGCTAGACATGAGG | |
| | | TCTATGGACTTCAAGAGCAACAGTGCTGTGGCCTGGAGCAACA | |
| | | AATCTGACTTTGCATGTGCAAACGCCTTCAACAACAGCATTAT | |
| | | TCCAGAAGACACCTTCTTCCCCAGCCCAGGTAAGGGCAGCTTT | |
| | | GGTGCCTTCGCAGGCTGTTTCCTTGCTTCAGGAATGGCCAGGT | |
| | | TCTGCCCAGAGCTCTGGTCAATGATGTCTAAAACTCCTCTGAT | |
| | | TGGTGGTCTCGGC | |
| | Between | TAGAACAGAGAAACAGGAGAATATGGGCCAAACAGGATATCTG | 128 |
| | Homology | TGGTAAGCAGTTCCTGCCCCGGCTCAGGGCCAAGAACAGTTGG | |
| | Arms | AACAGCAGAATATGGGCCAAACAGGATATCTGTGGTAAGCAGT | |
| | | TCCTGCCCCGGCTCAGGGCCAAGAACAGATGGTCCCCAGATGC | |
| | | GGTCCCGCCCTCAGCAGTTTCTAGAGAACCATCAGATGTTTCC | |
| | | AGGGTGCCCCAAGGACCTGAAATGACCCTGTGCCTTATTTGAA | |
| | | CTAACCAATCAGTTCGCTTCTCGCTTCTGTTCGCGCGCTTCTG | |
| | | CTCCCCGAGCTCTATATAAGCAGAGCTCGTTTAGTGAACCGTC | |
| | | AGATCGCCTGGAGACGCCATCCACGCTGTTTTGACTTCCATAG | |
| | | AAGGATCTCGCCGCCACCATGCCTCTGGGCCTGCTGTGGCTGG | |
| | | GCCTGGCCCTGCTGGGCGCCCTGCACGCCCAGGCCGGCGTGCA | |
| | | GGTGGAGACAATCTCCCCAGGCGACGGACGCACATTCCCTAAG | |
| | | CGGGGCCAGACCTGCGTGGTGCACTATACAGGCATGCTGGAGG | |
| | | ATGGCAAGAAGTTTGACAGCTCCCGGGATAGAAACAAGCCATT | |
| | | CAAGTTTATGCTGGGCAAGCAGGAAGTGATCAGAGGCTGGGAG | |
| | | GAGGGCGTGGCCCAGATGTCTGTGGGCCAGAGGGCCAAGCTGA | |
| | | CCATCAGCCCAGACTACGCCTATGGAGCAACAGGCCACCCAGG | |
| | | AATCATCCCACCTCACGCCACCCTGGTGTTCGATGTGGAGCTG | |
| | | CTGAAGCTGGGCGAGGGATCCAACACATCAAAAGAGAACCCCT | |
| | | TTCTGTTCGCATTGGAGGCCGTAGTCATATCTGTTGGATCCAT | |
| | | GGGACTTATTATCTCCCTGTTGTGTGTGTACTTCTGGCTGGAA | |
| | | CGGACTATGCCCAGGATCCCCACGCTCAAGAATCTGGAAGATC | |
| | | TCGTCACAGAATACCATGGTAATTTCAGCGCCTGGAGCGGAGT | |
| | | CTCTAAGGGTCTGGCCGAATCCCTCCAACCCGATTATTCTGAA | |
| | | CGGTTGTGCCTCGTATCCGAAATACCACCAAAAGGCGGGGCTC | |
| | | TGGGTGAGGGCCCAGGGGCGAGTCCGTGCAATCAACACAGCCC | |
| | | GTATTGGGCCCCTCCTTGTTATACGTTGAAGCCCGAAACTGGA | |
| | | TCCGGCGAGGGCAGAGGAAGTCTGCTAACATGCGGTGACGTCG | |
| | | AGGAGAATCCTGGACCTATGGGATCAAGACTGCTGTGCTGGGT | |
| | | GCTGCTGTGCCTGCTGGGAGCCGGACCTGTGAAAGCCGGGGTG | |
| | | ACTCAGACTCCACGATACCTGATCAAGACCAGGGGCCAGCAGG | |
| | | TGACACTGTCTTGCAGCCCAATCTCCGGACACCGCTCCGTGTC | |
| | | TTGGTACCAGCAGACCCCTGGACAGGGACTGCAGTTCCTGTTT | |
| | | GAGTATTTCTCTGAGACACAGCGGAACAAGGGCAATTTCCCCG | |
| | | GCCGGTTTAGCGGCAGACAGTTTAGCAACTCCAGGTCTGAGAT | |
| | | GAATGTGAGCACCCTGGAGCTGGGCGACTCCGCCCTGTACCTG | |
| | | TGCGCAAGCTCCCTGGCCCTGGGACAGGGGAATCAGCAGTTTT | |
| | | TCGGACCCGGAACAAGGCTGACCGTGCTGGAGGACCTTAAGAA | |
| | | TGTGTTCCCTCCCGAGGTGGCTGTCTTCGAACCAAGCGAAGCC | |
| | | GAGATCTCTCACACACAAAAGGCTACTCTCGTGTGTCTGGCAA | |
| | | CGGGTTTTTACCCTGATCATGTAGAGTTGTCTTGGTGGGTTAA | |
| | | CGGCAAAGAAGTACACTCCGGTGTATCTACCGATCCTCAGCCA | |
| | | CTTAAGGAACAACCCGCACTCAACGACTCCAGATACTGTTTGA | |
| | | GTTCTAGGCTGAGAGTCTCAGCGACGTTTTGGCAGAACCCACG | |
| | | GAATCATTTCAGATGTCAGGTCCAGTTTTACGGGTTGAGCGAG | |
| | | AACGACGAGTGGACACAGGATCGGGCTAAACCAGTGACCCAGA | |
| | | TTGTATCAGCCGAAGCATGGGGAAGGGCAGATTGCGGCTTTAC | |
| | | CTCAGAGTCATATCAACAGGGCGTTTTGTCTGCAACAATCCTT | |
| | | TATGAGATCCTGCTTGGTAAGGCGACTCTGTATGCCGTGCTTG | |
| | | TTAGTGCCCTGGTTCTGATGGCAATGGTAAAAAGAAAGGACTC | |
| | | TAGGGGAGGTTCTGGCGCTACTAACTTCAGCCTGCTGAAGCAG | |
| | | GCTGGAGACGTGGAGGAGAACCCTGGACCTATGAAGAGCCTGC | |
| | | GCGTGCTGCTGGTCATCCTGTGGCTGCAATTGAGTTGGGTGTG | |
| | | GAGCCAGAAGGACCAGGTGTTTCAGCCTAGCACTGTCGCATCA | |
| | | TCAGAGGGGGCCGTCGTGGAAATCTTTTGTAATCACAGCGTGT | |
| | | CCAACGCCTACAATTTCTTTTGGTATCTGCACTTCCCAGGATG | |
| | | CGCACCTAGGCTGCTGGTGAAGGGCTCTAAGCCCAGCCAGCAG | |
| | | GGCCGGTACAACATGACCTATGAGCGGTTCAGCTCCTCTCTGC | |
| | | TGATCCTGCAGGTGCGGGAGGCAGACGCAGCCGTGTACTATTG | |
| | | TGCCGTGGAGGATCTGAATCAGGCCGGCACAGCCCTGATTTTC | |
| | | GGCAAAGGAACAACCCTGAGCGTGAGCAGCAATATCCAGAACC | |
| | | CTGACCCTGCAGTATAT | |
| | Full-length | CCTATTAAATAAAAGAATAAGCAGTATTATTAAGTAGCCCTGC | 129 |
| | donor | ATTTCAGGTTTCCTTGAGTGGCAGGCCAGGCCTGGCCGTGAAC | |
| | template | GTTCACTGAAATCATGGCCTCTTGGCCAAGATTGATAGCTTGT | |
| | | GCCTGTCCCTGAGTCCCAGTCCATCACGAGCAGCTGGTTTCTA | |
| | | AGATGCTATTTCCCGTATAAAGCATGAGACCGTGACTTGCCAG | |
| | | CCCCACAGAGCCCCGCCCTTGTCCATCACTGGCATCTGGACTC | |
| | | CAGCCTGGGTTGGGGCAAAGAGGGAAATGAGATCATGTCCTAA | |
| | | CCCTGATCCTCTTGTCCCACAGATATCCAGAACCCTGACCCTG | |
| | | CCTAGAACAGAGAAACAGGAGAATATGGGCCAAACAGGATATC | |
| | | TGTGGTAAGCAGTTCCTGCCCCGGCTCAGGGCCAAGAACAGTT | |
| | | GGAACAGCAGAATATGGGCCAAACAGGATATCTGTGGTAAGCA | |
| | | GTTCCTGCCCCGGCTCAGGGCCAAGAACAGATGGTCCCCAGAT | |
| | | GCGGTCCCGCCCTCAGCAGTTTCTAGAGAACCATCAGATGTTT | |
| | | CCAGGGTGCCCCAAGGACCTGAAATGACCCTGTGCCTTATTTG | |
| | | AACTAACCAATCAGTTCGCTTCTCGCTTCTGTTCGCGCGCTTC | |
| | | TGCTCCCCGAGCTCTATATAAGCAGAGCTCGTTTAGTGAACCG | |
| | | TCAGATCGCCTGGAGACGCCATCCACGCTGTTTTGACTTCCAT | |
| | | AGAAGGATCTCGCCGCCACCATGCCTCTGGGCCTGCTGTGGCT | |
| | | GGGCCTGGCCCTGCTGGGCGCCCTGCACGCCCAGGCCGGCGTG | |
| | | CAGGTGGAGACAATCTCCCCAGGCGACGGACGCACATTCCCTA | |
| | | AGCGGGGCCAGACCTGCGTGGTGCACTATACAGGCATGCTGGA | |
| | | GGATGGCAAGAAGTTTGACAGCTCCCGGGATAGAAACAAGCCA | |
| | | TTCAAGTTTATGCTGGGCAAGCAGGAAGTGATCAGAGGCTGGG | |
| | | AGGAGGGCGTGGCCCAGATGTCTGTGGGCCAGAGGGCCAAGCT | |
| | | GACCATCAGCCCAGACTACGCCTATGGAGCAACAGGCCACCCA | |
| | | GGAATCATCCCACCTCACGCCACCCTGGTGTTCGATGTGGAGC | |
| | | TGCTGAAGCTGGGCGAGGGATCCAACACATCAAAAGAGAACCC | |
| | | CTTTCTGTTCGCATTGGAGGCCGTAGTCATATCTGTTGGATCC | |
| | | ATGGGACTTATTATCTCCCTGTTGTGTGTGTACTTCTGGCTGG | |
| | | AACGGACTATGCCCAGGATCCCCACGCTCAAGAATCTGGAAGA | |
| | | TCTCGTCACAGAATACCATGGTAATTTCAGCGCCTGGAGCGGA | |
| | | GTCTCTAAGGGTCTGGCCGAATCCCTCCAACCCGATTATTCTG | |
| | | AACGGTTGTGCCTCGTATCCGAAATACCACCAAAAGGCGGGGC | |
| | | TCTGGGTGAGGGCCCAGGGGCGAGTCCGTGCAATCAACACAGC | |
| | | CCGTATTGGGCCCCTCCTTGTTATACGTTGAAGCCCGAAACTG | |
| | | GATCCGGCGAGGGCAGAGGAAGTCTGCTAACATGCGGTGACGT | |
| | | CGAGGAGAATCCTGGACCTATGGGATCAAGACTGCTGTGCTGG | |
| | | GTGCTGCTGTGCCTGCTGGGAGCCGGACCTGTGAAAGCCGGGG | |
| | | TGACTCAGACTCCACGATACCTGATCAAGACCAGGGGCCAGCA | |
| | | GGTGACACTGTCTTGCAGCCCAATCTCCGGACACCGCTCCGTG | |
| | | TCTTGGTACCAGCAGACCCCTGGACAGGGACTGCAGTTCCTGT | |
| | | TTGAGTATTTCTCTGAGACACAGCGGAACAAGGGCAATTTCCC | |
| | | CGGCCGGTTTAGCGGCAGACAGTTTAGCAACTCCAGGTCTGAG | |
| | | ATGAATGTGAGCACCCTGGAGCTGGGCGACTCCGCCCTGTACC | |
| | | TGTGCGCAAGCTCCCTGGCCCTGGGACAGGGGAATCAGCAGTT | |
| | | TTTCGGACCCGGAACAAGGCTGACCGTGCTGGAGGACCTTAAG | |
| | | AATGTGTTCCCTCCCGAGGTGGCTGTCTTCGAACCAAGCGAAG | |
| | | CCGAGATCTCTCACACACAAAAGGCTACTCTCGTGTGTCTGGC | |
| | | AACGGGTTTTTACCCTGATCATGTAGAGTTGTCTTGGTGGGTT | |
| | | AACGGCAAAGAAGTACACTCCGGTGTATCTACCGATCCTCAGC | |
| | | CACTTAAGGAACAACCCGCACTCAACGACTCCAGATACTGTTT | |
| | | GAGTTCTAGGCTGAGAGTCTCAGCGACGTTTTGGCAGAACCCA | |
| | | CGGAATCATTTCAGATGTCAGGTCCAGTTTTACGGGTTGAGCG | |
| | | AGAACGACGAGTGGACACAGGATCGGGCTAAACCAGTGACCCA | |
| | | GATTGTATCAGCCGAAGCATGGGGAAGGGCAGATTGCGGCTTT | |
| | | ACCTCAGAGTCATATCAACAGGGCGTTTTGTCTGCAACAATCC | |
| | | TTTATGAGATCCTGCTTGGTAAGGCGACTCTGTATGCCGTGCT | |
| | | TGTTAGTGCCCTGGTTCTGATGGCAATGGTAAAAAGAAAGGAC | |
| | | TCTAGGGGAGGTTCTGGCGCTACTAACTTCAGCCTGCTGAAGC | |
| | | AGGCTGGAGACGTGGAGGAGAACCCTGGACCTATGAAGAGCCT | |
| | | GCGCGTGCTGCTGGTCATCCTGTGGCTGCAATTGAGTTGGGTG | |
| | | TGGAGCCAGAAGGACCAGGTGTTTCAGCCTAGCACTGTCGCAT | |
| | | CATCAGAGGGGGCCGTCGTGGAAATCTTTTGTAATCACAGCGT | |
| | | GTCCAACGCCTACAATTTCTTTTGGTATCTGCACTTCCCAGGA | |
| | | TGCGCACCTAGGCTGCTGGTGAAGGGCTCTAAGCCCAGCCAGC | |
| | | AGGGCCGGTACAACATGACCTATGAGCGGTTCAGCTCCTCTCT | |
| | | GCTGATCCTGCAGGTGCGGGAGGCAGACGCAGCCGTGTACTAT | |
| | | TGTGCCGTGGAGGATCTGAATCAGGCCGGCACAGCCCTGATTT | |
| | | TCGGCAAAGGAACAACCCTGAGCGTGAGCAGCAATATCCAGAA | |
| | | CCCTGACCCTGCAGTATATCAGCTGAGAGACTCTAAATCCAGT | |
| | | GACAAGTCTGTCTGCCTATTCACCGATTTTGATTCTCAAACAA | |
| | | ATGTGTCACAAAGTAAGGATTCTGATGTGTATATCACAGACAA | |
| | | AACTGTGCTAGACATGAGGTCTATGGACTTCAAGAGCAACAGT | |
| | | GCTGTGGCCTGGAGCAACAAATCTGACTTTGCATGTGCAAACG | |
| | | CCTTCAACAACAGCATTATTCCAGAAGACACCTTCTTCCCCAG | |
| | | CCCAGGTAAGGGCAGCTTTGGTGCCTTCGCAGGCTGTTTCCTT | |
| | | GCTTCAGGAATGGCCAGGTTCTGCCCAGAGCTCTGGTCAATGA | |
| | | TGTCTAAAACTCCTCTGATTGGTGGTCTCGGC | |
| T1D5-1 | 5′ Homology | CCTATTAAATAAAAGAATAAGCAGTATTATTAAGTAGCCCTGC | 130 |
| | Arm | ATTTCAGGTTTCCTTGAGTGGCAGGCCAGGCCTGGCCGTGAAC | |
| | | GTTCACTGAAATCATGGCCTCTTGGCCAAGATTGATAGCTTGT | |
| | | GCCTGTCCCTGAGTCCCAGTCCATCACGAGCAGCTGGTTTCTA | |
| | | AGATGCTATTTCCCGTATAAAGCATGAGACCGTGACTTGCCAG | |
| | | CCCCACAGAGCCCCGCCCTTGTCCATCACTGGCATCTGGACTC | |
| | | CAGCCTGGGTTGGGGCAAAGAGGGAAATGAGATCATGTCCTAA | |
| | | CCCTGATCCTCTTGTCCCACAGATATCCAGAACCCTGACCCTG | |
| | | CC | |
| | Intervening | TA | |
| | sequence | | |
| | MND | GAACAGAGAAACAGGAGAATATGGGCCAAACAGGATATCTGTG | 131 |
| | Promoter | GTAAGCAGTTCCTGCCCCGGCTCAGGGCCAAGAACAGTTGGAA | |
| | | CAGCAGAATATGGGCCAAACAGGATATCTGTGGTAAGCAGTTC | |
| | | CTGCCCCGGCTCAGGGCCAAGAACAGATGGTCCCCAGATGCGG | |
| | | TCCCGCCCTCAGCAGTTTCTAGAGAACCATCAGATGTTTCCAG | |
| | | GGTGCCCCAAGGACCTGAAATGACCCTGTGCCTTATTTGAACT | |
| | | AACCAATCAGTTCGCTTCTCGCTTCTGTTCGCGCGCTTCTGCT | |
| | | CCCCGAGCTCTATATAAGCAGAGCTCGTTTAGTGAACCGTCAG | |
| | | ATC | |
| | Intervening | GCCTGGAGACGCCATCCACGCTGTTTTGACTTCCATAGAAGGA | 132 |
| | Sequence | TCTCGCCGCCACCATGCCTCTGGGCCTGCTGTGGCTGGGCCTG | |
| | (LCN2 | GCCCTGCTGGGCGCCCTGCACGCCCAGGCC | |
| | signal | | |
| | peptide of | | |
| | FKBP-IL2Rg | | |
| | underlined | | |
| | FKBP-IL2Rg | GGCGTGCAGGTGGAGACAATCTCCCCAGGCGACGGACGCACAT | 133 |
| | | TCCCTAAGCGGGGCCAGACCTGCGTGGTGCACTATACAGGCAT | |
| | | GCTGGAGGATGGCAAGAAGTTTGACAGCTCCCGGGATAGAAAC | |
| | | AAGCCATTCAAGTTTATGCTGGGCAAGCAGGAAGTGATCAGAG | |
| | | GCTGGGAGGAGGGCGTGGCCCAGATGTCTGTGGGCCAGAGGGC | |
| | | CAAGCTGACCATCAGCCCAGACTACGCCTATGGAGCAACAGGC | |
| | | CACCCAGGAATCATCCCACCTCACGCCACCCTGGTGTTCGATG | |
| | | TGGAGCTGCTGAAGCTGGGCGAGGGATCCAACACATCAAAAGA | |
| | | GAACCCCTTTCTGTTCGCATTGGAGGCCGTAGTCATATCTGTT | |
| | | GGATCCATGGGACTTATTATCTCCCTGTTGTGTGTGTACTTCT | |
| | | GGCTGGAACGGACTATGCCCAGGATCCCCACGCTCAAGAATCT | |
| | | GGAAGATCTCGTCACAGAATACCATGGTAATTTCAGCGCCTGG | |
| | | AGCGGAGTCTCTAAGGGTCTGGCCGAATCCCTCCAACCCGATT | |
| | | ATTCTGAACGGTTGTGCCTCGTATCCGAAATACCACCAAAAGG | |
| | | CGGGGCTCTGGGTGAGGGCCCAGGGGCGAGTCCGTGCAATCAA | |
| | | CACAGCCCGTATTGGGCCCCTCCTTGTTATACGTTGAAGCCCG | |
| | | AAACT | |
| | GSG linker | GGATCCGGC | |
| | T2A | GAGGGCAGAGGAAGTCTGCTAACATGCGGTGACGTCGAGGAGA | 134 |
| | | ATCCTGGACCT | |
| | T1D5-1 TCRβ | ATGGGCTGCAGGCTGCTCTGCTGTGCGGTTCTCTGTCTCCTGG | 135 |
| | | GAGCGGTCCCCATGGAAACGGGAGTTACGCAGACACCAAGACA | |
| | | CCTGGTCATGGGAATGACAAATAAGAAGTCTTTGAAATGTGAA | |
| | | CAACATCTGGGTCATAACGCTATGTATTGGTACAAGCAAAGTG | |
| | | CTAAGAAGCCACTGGAGCTCATGTTTGTCTACAGTCTTGAAGA | |
| | | ACGGGTTGAAAACAACAGTGTGCCAAGTCGCTTCTCACCTGAA | |
| | | TGCCCCAACAGCTCTCACTTATTCCTTCACCTACACACCCTGC | |
| | | AGCCAGAAGACTCGGCCCTGTATCTCTGCGCCAGCAGCCAAGA | |
| | | AGTAGGTACAGTCCCCAATCAGCCCCAGCATTTTGGTGATGGG | |
| | | ACTCGACTCTCCATCCTAGAGGACCTTAATAAGGTGTTCCCTC | |
| | | CCGAGGTGGCTGTCTTCGAACCAAGCGAAGCCGAGATCTCTCA | |
| | | CACACAAAAGGCTACTCTCGTGTGTCTGGCAACGGGTTTTTTC | |
| | | CCTGATCATGTAGAGTTGTCTTGGTGGGTTAACGGCAAAGAAG | |
| | | TACACTCCGGTGTAAGCACCGATCCTCAGCCACTTAAGGAACA | |
| | | ACCCGCACTCAACGACTCCAGATACTGTTTGAGTTCTAGGCTG | |
| | | AGAGTCTCAGCGACGTTTTGGCAGAACCCACGGAATCATTTCA | |
| | | GATGTCAGGTCCAGTTTTACGGGTTGAGCGAGAACGACGAGTG | |
| | | GACACAGGATCGGGCTAAACCAGTGACCCAGATTGTATCAGCC | |
| | | GAAGCATGGGGAAGGGCAGATTGCGGCTTTACCTCAGTTTCAT | |
| | | ATCAACAGGGCGTTTTGTCTGCAACAATCCTTTATGAGATCCT | |
| | | GCTTGGTAAGGCGACTCTGTATGCCGTGCTTGTTAGTGCCCTG | |
| | | GTTCTGATGGCAATGGTAAAAAGAAAGGACTTT | |
| | GSG linker | GGTTCTGGC | |
| | P2A | GCTACTAACTTCAGCCTGCTGAAGCAGGCTGGAGACGTGGAGG | 136 |
| | | AGAACCCTGGACCT | |
| | T1D5-1 TCRα | ATGCTACTCATCACATCAATGTTGGTCTTATGGATGCAATTGT | 137 |
| | Vα domain | CACAGGTGAATGGACAACAGGTAATGCAAATTCCTCAGTACCA | |
| | and C | GCATGTACAAGAAGGAGAGGACTTCACCACGTACTGCAATTCC | |
| | domain | TCAACTACTTTAAGCAATATACAGTGGTATAAGCAAAGGCCTG | |
| | portion | GTGGACATCCCGTTTTTTTGATACAGTTAGTGAAGAGTGGAGA | |
| | | AGTGAAGAAGCAGAAAAGACTGACATTTCAGTTTGGAGAAGCA | |
| | | AAAAAGAACAGCTCCCTGCACATCACAGCCACCCAGACTACAG | |
| | | ATGTAGGAACCTACTTCTGTGCAGGTCAAACTGGGGCAAACAA | |
| | | CCTCTTCTTTGGGACTGGAACGAGACTCACCGTTATTCCCTAT | |
| | | ATCCAGAACCCTGACCCTGCAGTATAT | |
| | 3′ Homology | CAGCTGAGAGACTCTAAATCCAGTGACAAGTCTGTCTGCCTAT | 138 |
| | Arm | TCACCGATTTTGATTCTCAAACAAATGTGTCACAAAGTAAGGA | |
| | | TTCTGATGTGTATATCACAGACAAAACTGTGCTAGACATGAGG | |
| | | TCTATGGACTTCAAGAGCAACAGTGCTGTGGCCTGGAGCAACA | |
| | | AATCTGACTTTGCATGTGCAAACGCCTTCAACAACAGCATTAT | |
| | | TCCAGAAGACACCTTCTTCCCCAGCCCAGGTAAGGGCAGCTTT | |
| | | GGTGCCTTCGCAGGCTGTTTCCTTGCTTCAGGAATGGCCAGGT | |
| | | TCTGCCCAGAGCTCTGGTCAATGATGTCTAAAACTCCTCTGAT | |
| | | TGGTGGTCTCGGC | |
| | Between | TAGAACAGAGAAACAGGAGAATATGGGCCAAACAGGATATCTG | 139 |
| | Homology | TGGTAAGCAGTTCCTGCCCCGGCTCAGGGCCAAGAACAGTTGG | |
| | Arms | AACAGCAGAATATGGGCCAAACAGGATATCTGTGGTAAGCAGT | |
| | | TCCTGCCCCGGCTCAGGGCCAAGAACAGATGGTCCCCAGATGC | |
| | | GGTCCCGCCCTCAGCAGTTTCTAGAGAACCATCAGATGTTTCC | |
| | | AGGGTGCCCCAAGGACCTGAAATGACCCTGTGCCTTATTTGAA | |
| | | CTAACCAATCAGTTCGCTTCTCGCTTCTGTTCGCGCGCTTCTG | |
| | | CTCCCCGAGCTCTATATAAGCAGAGCTCGTTTAGTGAACCGTC | |
| | | AGATCGCCTGGAGACGCCATCCACGCTGTTTTGACTTCCATAG | |
| | | AAGGATCTCGCCGCCACCATGCCTCTGGGCCTGCTGTGGCTGG | |
| | | GCCTGGCCCTGCTGGGCGCCCTGCACGCCCAGGCCGGCGTGCA | |
| | | GGTGGAGACAATCTCCCCAGGCGACGGACGCACATTCCCTAAG | |
| | | CGGGGCCAGACCTGCGTGGTGCACTATACAGGCATGCTGGAGG | |
| | | ATGGCAAGAAGTTTGACAGCTCCCGGGATAGAAACAAGCCATT | |
| | | CAAGTTTATGCTGGGCAAGCAGGAAGTGATCAGAGGCTGGGAG | |
| | | GAGGGCGTGGCCCAGATGTCTGTGGGCCAGAGGGCCAAGCTGA | |
| | | CCATCAGCCCAGACTACGCCTATGGAGCAACAGGCCACCCAGG | |
| | | AATCATCCCACCTCACGCCACCCTGGTGTTCGATGTGGAGCTG | |
| | | CTGAAGCTGGGCGAGGGATCCAACACATCAAAAGAGAACCCCT | |
| | | TTCTGTTCGCATTGGAGGCCGTAGTCATATCTGTTGGATCCAT | |
| | | GGGACTTATTATCTCCCTGTTGTGTGTGTACTTCTGGCTGGAA | |
| | | CGGACTATGCCCAGGATCCCCACGCTCAAGAATCTGGAAGATC | |
| | | TCGTCACAGAATACCATGGTAATTTCAGCGCCTGGAGCGGAGT | |
| | | CTCTAAGGGTCTGGCCGAATCCCTCCAACCCGATTATTCTGAA | |
| | | CGGTTGTGCCTCGTATCCGAAATACCACCAAAAGGCGGGGCTC | |
| | | TGGGTGAGGGCCCAGGGGCGAGTCCGTGCAATCAACACAGCCC | |
| | | GTATTGGGCCCCTCCTTGTTATACGTTGAAGCCCGAAACTGGA | |
| | | TCCGGCGAGGGCAGAGGAAGTCTGCTAACATGCGGTGACGTCG | |
| | | AGGAGAATCCTGGACCTATGGGCTGCAGGCTGCTCTGCTGTGC | |
| | | GGTTCTCTGTCTCCTGGGAGCGGTCCCCATGGAAACGGGAGTT | |
| | | ACGCAGACACCAAGACACCTGGTCATGGGAATGACAAATAAGA | |
| | | AGTCTTTGAAATGTGAACAACATCTGGGTCATAACGCTATGTA | |
| | | TTGGTACAAGCAAAGTGCTAAGAAGCCACTGGAGCTCATGTTT | |
| | | GTCTACAGTCTTGAAGAACGGGTTGAAAACAACAGTGTGCCAA | |
| | | GTCGCTTCTCACCTGAATGCCCCAACAGCTCTCACTTATTCCT | |
| | | TCACCTACACACCCTGCAGCCAGAAGACTCGGCCCTGTATCTC | |
| | | TGCGCCAGCAGCCAAGAAGTAGGTACAGTCCCCAATCAGCCCC | |
| | | AGCATTTTGGTGATGGGACTCGACTCTCCATCCTAGAGGACCT | |
| | | TAATAAGGTGTTCCCTCCCGAGGTGGCTGTCTTCGAACCAAGC | |
| | | GAAGCCGAGATCTCTCACACACAAAAGGCTACTCTCGTGTGTC | |
| | | TGGCAACGGGTTTTTTCCCTGATCATGTAGAGTTGTCTTGGTG | |
| | | GGTTAACGGCAAAGAAGTACACTCCGGTGTAAGCACCGATCCT | |
| | | CAGCCACTTAAGGAACAACCCGCACTCAACGACTCCAGATACT | |
| | | GTTTGAGTTCTAGGCTGAGAGTCTCAGCGACGTTTTGGCAGAA | |
| | | CCCACGGAATCATTTCAGATGTCAGGTCCAGTTTTACGGGTTG | |
| | | AGCGAGAACGACGAGTGGACACAGGATCGGGCTAAACCAGTGA | |
| | | CCCAGATTGTATCAGCCGAAGCATGGGGAAGGGCAGATTGCGG | |
| | | CTTTACCTCAGTTTCATATCAACAGGGCGTTTTGTCTGCAACA | |
| | | ATCCTTTATGAGATCCTGCTTGGTAAGGCGACTCTGTATGCCG | |
| | | TGCTTGTTAGTGCCCTGGTTCTGATGGCAATGGTAAAAAGAAA | |
| | | GGACTTTGGTTCTGGCGCTACTAACTTCAGCCTGCTGAAGCAG | |
| | | GCTGGAGACGTGGAGGAGAACCCTGGACCTATGCTACTCATCA | |
| | | CATCAATGTTGGTCTTATGGATGCAATTGTCACAGGTGAATGG | |
| | | ACAACAGGTAATGCAAATTCCTCAGTACCAGCATGTACAAGAA | |
| | | GGAGAGGACTTCACCACGTACTGCAATTCCTCAACTACTTTAA | |
| | | GCAATATACAGTGGTATAAGCAAAGGCCTGGTGGACATCCCGT | |
| | | TTTTTTGATACAGTTAGTGAAGAGTGGAGAAGTGAAGAAGCAG | |
| | | AAAAGACTGACATTTCAGTTTGGAGAAGCAAAAAAGAACAGCT | |
| | | CCCTGCACATCACAGCCACCCAGACTACAGATGTAGGAACCTA | |
| | | CTTCTGTGCAGGTCAAACTGGGGCAAACAACCTCTTCTTTGGG | |
| | | ACTGGAACGAGACTCACCGTTATTCCCTATATCCAGAACCCTG | |
| | | ACCCTGCAGTATAT | |
| | Full-length | CCTATTAAATAAAAGAATAAGCAGTATTATTAAGTAGCCCTGC | 140 |
| | donor | ATTTCAGGTTTCCTTGAGTGGCAGGCCAGGCCTGGCCGTGAAC | |
| | template | GTTCACTGAAATCATGGCCTCTTGGCCAAGATTGATAGCTTGT | |
| | | GCCTGTCCCTGAGTCCCAGTCCATCACGAGCAGCTGGTTTCTA | |
| | | AGATGCTATTTCCCGTATAAAGCATGAGACCGTGACTTGCCAG | |
| | | CCCCACAGAGCCCCGCCCTTGTCCATCACTGGCATCTGGACTC | |
| | | CAGCCTGGGTTGGGGCAAAGAGGGAAATGAGATCATGTCCTAA | |
| | | CCCTGATCCTCTTGTCCCACAGATATCCAGAACCCTGACCCTG | |
| | | CCTAGAACAGAGAAACAGGAGAATATGGGCCAAACAGGATATC | |
| | | TGTGGTAAGCAGTTCCTGCCCCGGCTCAGGGCCAAGAACAGTT | |
| | | GGAACAGCAGAATATGGGCCAAACAGGATATCTGTGGTAAGCA | |
| | | GTTCCTGCCCCGGCTCAGGGCCAAGAACAGATGGTCCCCAGAT | |
| | | GCGGTCCCGCCCTCAGCAGTTTCTAGAGAACCATCAGATGTTT | |
| | | CCAGGGTGCCCCAAGGACCTGAAATGACCCTGTGCCTTATTTG | |
| | | AACTAACCAATCAGTTCGCTTCTCGCTTCTGTTCGCGCGCTTC | |
| | | TGCTCCCCGAGCTCTATATAAGCAGAGCTCGTTTAGTGAACCG | |
| | | TCAGATCGCCTGGAGACGCCATCCACGCTGTTTTGACTTCCAT | |
| | | AGAAGGATCTCGCCGCCACCATGCCTCTGGGCCTGCTGTGGCT | |
| | | GGGCCTGGCCCTGCTGGGCGCCCTGCACGCCCAGGCCGGCGTG | |
| | | CAGGTGGAGACAATCTCCCCAGGCGACGGACGCACATTCCCTA | |
| | | AGCGGGGCCAGACCTGCGTGGTGCACTATACAGGCATGCTGGA | |
| | | GGATGGCAAGAAGTTTGACAGCTCCCGGGATAGAAACAAGCCA | |
| | | TTCAAGTTTATGCTGGGCAAGCAGGAAGTGATCAGAGGCTGGG | |
| | | AGGAGGGCGTGGCCCAGATGTCTGTGGGCCAGAGGGCCAAGCT | |
| | | GACCATCAGCCCAGACTACGCCTATGGAGCAACAGGCCACCCA | |
| | | GGAATCATCCCACCTCACGCCACCCTGGTGTTCGATGTGGAGC | |
| | | TGCTGAAGCTGGGCGAGGGATCCAACACATCAAAAGAGAACCC | |
| | | CTTTCTGTTCGCATTGGAGGCCGTAGTCATATCTGTTGGATCC | |
| | | ATGGGACTTATTATCTCCCTGTTGTGTGTGTACTTCTGGCTGG | |
| | | AACGGACTATGCCCAGGATCCCCACGCTCAAGAATCTGGAAGA | |
| | | TCTCGTCACAGAATACCATGGTAATTTCAGCGCCTGGAGCGGA | |
| | | GTCTCTAAGGGTCTGGCCGAATCCCTCCAACCCGATTATTCTG | |
| | | AACGGTTGTGCCTCGTATCCGAAATACCACCAAAAGGCGGGGC | |
| | | TCTGGGTGAGGGCCCAGGGGCGAGTCCGTGCAATCAACACAGC | |
| | | CCGTATTGGGCCCCTCCTTGTTATACGTTGAAGCCCGAAACTG | |
| | | GATCCGGCGAGGGCAGAGGAAGTCTGCTAACATGCGGTGACGT | |
| | | CGAGGAGAATCCTGGACCTATGGGCTGCAGGCTGCTCTGCTGT | |
| | | GCGGTTCTCTGTCTCCTGGGAGCGGTCCCCATGGAAACGGGAG | |
| | | TTACGCAGACACCAAGACACCTGGTCATGGGAATGACAAATAA | |
| | | GAAGTCTTTGAAATGTGAACAACATCTGGGTCATAACGCTATG | |
| | | TATTGGTACAAGCAAAGTGCTAAGAAGCCACTGGAGCTCATGT | |
| | | TTGTCTACAGTCTTGAAGAACGGGTTGAAAACAACAGTGTGCC | |
| | | AAGTCGCTTCTCACCTGAATGCCCCAACAGCTCTCACTTATTC | |
| | | CTTCACCTACACACCCTGCAGCCAGAAGACTCGGCCCTGTATC | |
| | | TCTGCGCCAGCAGCCAAGAAGTAGGTACAGTCCCCAATCAGCC | |
| | | CCAGCATTTTGGTGATGGGACTCGACTCTCCATCCTAGAGGAC | |
| | | CTTAATAAGGTGTTCCCTCCCGAGGTGGCTGTCTTCGAACCAA | |
| | | GCGAAGCCGAGATCTCTCACACACAAAAGGCTACTCTCGTGTG | |
| | | TCTGGCAACGGGTTTTTTCCCTGATCATGTAGAGTTGTCTTGG | |
| | | TGGGTTAACGGCAAAGAAGTACACTCCGGTGTAAGCACCGATC | |
| | | CTCAGCCACTTAAGGAACAACCCGCACTCAACGACTCCAGATA | |
| | | CTGTTTGAGTTCTAGGCTGAGAGTCTCAGCGACGTTTTGGCAG | |
| | | AACCCACGGAATCATTTCAGATGTCAGGTCCAGTTTTACGGGT | |
| | | TGAGCGAGAACGACGAGTGGACACAGGATCGGGCTAAACCAGT | |
| | | GACCCAGATTGTATCAGCCGAAGCATGGGGAAGGGCAGATTGC | |
| | | GGCTTTACCTCAGTTTCATATCAACAGGGCGTTTTGTCTGCAA | |
| | | CAATCCTTTATGAGATCCTGCTTGGTAAGGCGACTCTGTATGC | |
| | | CGTGCTTGTTAGTGCCCTGGTTCTGATGGCAATGGTAAAAAGA | |
| | | AAGGACTTTGGTTCTGGCGCTACTAACTTCAGCCTGCTGAAGC | |
| | | AGGCTGGAGACGTGGAGGAGAACCCTGGACCTATGCTACTCAT | |
| | | CACATCAATGTTGGTCTTATGGATGCAATTGTCACAGGTGAAT | |
| | | GGACAACAGGTAATGCAAATTCCTCAGTACCAGCATGTACAAG | |
| | | AAGGAGAGGACTTCACCACGTACTGCAATTCCTCAACTACTTT | |
| | | AAGCAATATACAGTGGTATAAGCAAAGGCCTGGTGGACATCCC | |
| | | GTTTTTTTGATACAGTTAGTGAAGAGTGGAGAAGTGAAGAAGC | |
| | | AGAAAAGACTGACATTTCAGTTTGGAGAAGCAAAAAAGAACAG | |
| | | CTCCCTGCACATCACAGCCACCCAGACTACAGATGTAGGAACC | |
| | | TACTTCTGTGCAGGTCAAACTGGGGCAAACAACCTCTTCTTTG | |
| | | GGACTGGAACGAGACTCACCGTTATTCCCTATATCCAGAACCC | |
| | | TGACCCTGCAGTATATCAGCTGAGAGACTCTAAATCCAGTGAC | |
| | | AAGTCTGTCTGCCTATTCACCGATTTTGATTCTCAAACAAATG | |
| | | TGTCACAAAGTAAGGATTCTGATGTGTATATCACAGACAAAAC | |
| | | TGTGCTAGACATGAGGTCTATGGACTTCAAGAGCAACAGTGCT | |
| | | GTGGCCTGGAGCAACAAATCTGACTTTGCATGTGCAAACGCCT | |
| | | TCAACAACAGCATTATTCCAGAAGACACCTTCTTCCCCAGCCC | |
| | | AGGTAAGGGCAGCTTTGGTGCCTTCGCAGGCTGTTTCCTTGCT | |
| | | TCAGGAATGGCCAGGTTCTGCCCAGAGCTCTGGTCAATGATGT | |
| | | CTAAAACTCCTCTGATTGGTGGTCTCGGC | |

TABLE 6
Examples of gRNAs for targeted
cleavage in a TRAC locus

| | Protospacer | SEQ | Protospacer |
|---|---|---|---|
| gRNA | Sequence | ID | adjacent |
| # | of gRNA | NO: | motif (PAM) |
| 1 | UCUCUCAGCUGGUACACGGC | 240 | AGG |

TABLE 7
Examples of nucleic acids for insertion into a FOXP3 locus

| | Encoded | | SEQ |
|---|---|---|---|
| Nucleic | Sequence | | ID |
| Acid # | Name | Nucleotide Sequence | NO: |
| FoxP3_v1 | 5′ Homology | ACTTGCCAGGACTGTTACAATAGCCTCCTCACTAGCCCCACTC | 141 |
| | Arm | ACAGCAGCCAGATGAATCTTTTGAGTCCATGCCTAGTCACTGG | |
| | | GGCAAAATAGGACTCCGAGGAGAAAGTCCGAGACCAGCTCCGG | |
| | | CAAGATGAGCAAACACAGCCTGTGCAGGGTGCAGGGAGGGCTA | |
| | | GAGGCCTGAGGCTTGAAACAGCTCTCAAGTGGAGGGGGAAACA | |
| | | ACCATTGCCCTCATAGAGGACACATCCACACCAGGGCTGTGCT | |
| | | AGCGTGGGCAGGCAAGCCAGGTGCTGGACCTCTGCACGTGGGG | |
| | | CATGTGTGGGTATGTACATGTACCTGTGTTCTTGGTGTGTGTG | |
| | | TGTGTGTGTGTGTGTGTGTGTGTCTAGAGCTGGGGTGCAACTA | |
| | | TGGGGCCCCTCGGGACATGTCCCAGCCAATGCCTGCTTTGACC | |
| | | AGAGGAGTGTCCACGTGGCTCAGGTGGTCGAGTATCTCATACC | |
| | | GCCCTAGCACACGTGTGACTCCTTTCCCCTATTGTCTACGCAG | |
| | | CCTGCCCTTGGACAAGGACCCGATGCCCAACCCCAGGCCTGGC | |
| | | AAGCCCTCGGCCCCTTCCTTGGCCCTTGGCCCATCCCCAGGA | |
| | Intervening | T | |
| | sequence | | |
| | MND Promoter | GAACAGAGAAACAGGAGAATATGGGCCAAACAGGATATCTGTG | 142 |
| | | GTAAGCAGTTCCTGCCCCGGCTCAGGGCCAAGAACAGTTGGAA | |
| | | CAGCAGAATATGGGCCAAACAGGATATCTGTGGTAAGCAGTTC | |
| | | CTGCCCCGGCTCAGGGCCAAGAACAGATGGTCCCCAGATGCGG | |
| | | TCCCGCCCTCAGCAGTTTCTAGAGAACCATCAGATGTTTCCAG | |
| | | GGTGCCCCAAGGACCTGAAATGACCCTGTGCCTTATTTGAACT | |
| | | AACCAATCAGTTCGCTTCTCGCTTCTGTTCGCGCGCTTCTGCT | |
| | | CCCCGAGCTCTATATAAGCAGAGCTCGTTTAGTGAACCGTCAG | |
| | | ATC | |
| | Intervening | GCCTGGAGACGCCATCCACGCTGTTTTGACTTCCATAGAAGGA | 143 |
| | Sequence | TCTCGAGGCCACCATGCCTCTGGGCCTGCTGTGGCTGGGCCTG | |
| | (LCN2 signal | GCCCTGCTGGGCGCCCTGCACGCCCAGGCC | |
| | peptide of | | |
| | FRB-IL2Rb | | |
| | underlined) | | |
| | FRB-IL2Rb | GAGATGTGGCACGAGGGCCTGGAGGAGGCCAGCAGGCTGTATT | 144 |
| | | TTGGCGAGCGCAACGTGAAGGGCATGTTCGAGGTGCTGGAGCC | |
| | | TCTGCACGCCATGATGGAGAGAGGCCCACAGACCCTGAAGGAG | |
| | | ACATCCTTTAACCAGGCCTATGGACGGGACCTGATGGAGGCAC | |
| | | AGGAGTGGTGCAGAAAGTACATGAAGTCTGGCAATGTGAAGGA | |
| | | CCTGACGCAGGCCTGGGATCTGTACTATCACGTGTTTCGGAGA | |
| | | ATCTCCAAGGGCAAAGACACGATTCCGTGGCTTGGGCATCTGC | |
| | | TCGTTGGGCTGAGTGGTGCGTTTGGTTTCATCATCTTGGTCTA | |
| | | TCTCTTGATCAATTGCAGAAATACAGGCCCTTGGCTGAAAAAA | |
| | | GTGCTCAAGTGTAATACCCCCGACCCAAGCAAGTTCTTCTCCC | |
| | | AGCTTTCTTCAGAGCATGGAGGCGATGTGCAGAAATGGCTCTC | |
| | | TTCACCTTTTCCCTCCTCAAGTTTCTCCCCGGGAGGGCTGGCG | |
| | | CCCGAGATTTCACCTCTTGAGGTACTTGAACGAGACAAGGTTA | |
| | | CCCAACTTCTCCTTCAACAGGATAAGGTACCCGAACCTGCGAG | |
| | | CCTTAGCTCCAACCACTCTCTTACGAGCTGCTTCACCAATCAG | |
| | | GGATACTTCTTTTTCCACCTTCCCGATGCGCTGGAAATCGAAG | |
| | | CATGTCAAGTTTACTTTACCTATGATCCATATAGCGAGGAAGA | |
| | | TCCCGACGAAGGAGTCGCCGGTGCGCCCACGGGTTCCTCACCC | |
| | | CAACCTCTCCAGCCTCTCTCAGGAGAAGATGATGCTTATTGCA | |
| | | CTTTTCCCAGTAGAGACGATCTCCTCCTCTTTTCTCCATCTCT | |
| | | TTTGGGGGGACCTTCCCCCCCTTCTACGGCACCTGGCGGGTCT | |
| | | GGTGCTGGCGAGGAGCGGATGCCGCCGTCCCTCCAGGAGCGAG | |
| | | TACCACGAGATTGGGATCCCCAGCCACTTGGACCACCAACTCC | |
| | | AGGCGTACCTGACCTTGTCGATTTTCAACCTCCCCCTGAATTG | |
| | | GTGCTGCGAGAGGCTGGGGAGGAAGTTCCGGACGCTGGGCCGA | |
| | | GGGAGGGCGTGTCCTTTCCATGGAGTAGGCCTCCAGGTCAAGG | |
| | | CGAGTTTAGGGCTCTCAACGCGCGGCTGCCGTTGAATACAGAC | |
| | | GCTTATCTCTCACTGCAGGAACTGCAAGGTCAGGACCCAACAC | |
| | | ATCTTGTA | |
| | GSG linker | GGATCCGGC | |
| | P2A | GCCACCAACTTTAGTCTGCTCAAGCAAGCCGGGGACGTCGAGG | 145 |
| | | AAAATCCTGGGCCA | |
| | Cytosolic | GAAATGTGGCACGAAGGACTCGAGGAAGCCAGTCGGCTGTATT | 146 |
| | FRB | TTGGCGAGCGGAATGTGAAAGGGATGTTTGAAGTGCTCGAGCC | |
| | | TCTCCACGCTATGATGGAACGGGGACCCCAGACTCTCAAAGAA | |
| | | ACCAGCTTTAATCAGGCTTACGGACGCGACCTCATGGAAGCTC | |
| | | AAGAATGGTGTAGAAAGTATATGAAGAGTGGCAACGTGAAAGA | |
| | | TCTGACACAAGCCTGGGATCTCTATTATCACGTGTTCAGACGC | |
| | | ATCAGCAAA | |
| | GSG linker | GGCAGCGGC | |
| | Second P2A | GCCACAAATTTCTCCCTGCTGAAACAGGCCGGCGACGTGGAAG | 147 |
| | | AGAATCCCGGACCT | |
| | FoxP3 | ATGCCCAATCCTAGACCTGGCAAGCCCAGCGCTCCTTCTCTTG | 148 |
| | portion | CTCTTGGACCTTCTCCTGGTGCA | |
| | 3′ Homology | TCGCCCAGCTGGAGGGCTGCACCCAAAGCCTCAGACCTGCTGG | 149 |
| | Arm | GGGCCCGGGGCCCAGGGGGAACCTTCCAGGGCCGAGATCTTCG | |
| | | AGGCGGGGCCCATGCCTCCTCTTCTTCCTTGAACCCCATGCCA | |
| | | CCATCGCAGCTGCAGGTGAGGCCCTGGGCCCAGGATGGGGCAG | |
| | | GCAGGGTGGGGTACCTGGACCTACAGGTGCCGACCTTTACTGT | |
| | | GGCACTGGGCGGGAGGGGGGCTGGCTGGGGCACAGGAAGTGGT | |
| | | TTCTGGGTCCCAGGCAAGTCTGTGACTTATGCAGATGTTGCAG | |
| | | GGCCAAGAAAATCCCCACCTGCCAGGCCTCAGAGATTGGAGGC | |
| | | TCTCCCCGACCTCCCAATCCCTGTCTCAGGAGAGGAGGAGGCC | |
| | | GTATTGTAGTCCCATGAGCATAGCTATGTGTCCCCATCCCCAT | |
| | | GTGACAAGAGAAGAGGACTGGGGCCAAGTAGGTGAGGTGACAG | |
| | | GGCTGAGGCCAGCTCTGCAACTTATTAGCTGTTTGATCTTTAA | |
| | | AAAGTTACTCGATCTCCATGAGCCTCAGTTTCCATACGTGTAA | |
| | | AAGGGGGATGATCATAGCATCTACCATGTGGGCTTGCAGTG | |
| | Between | TGAACAGAGAAACAGGAGAATATGGGCCAAACAGGATATCTGT | 150 |
| | Homology | GGTAAGCAGTTCCTGCCCCGGCTCAGGGCCAAGAACAGTTGGA | |
| | Arms | ACAGCAGAATATGGGCCAAACAGGATATCTGTGGTAAGCAGTT | |
| | | CCTGCCCCGGCTCAGGGCCAAGAACAGATGGTCCCCAGATGCG | |
| | | GTCCCGCCCTCAGCAGTTTCTAGAGAACCATCAGATGTTTCCA | |
| | | GGGTGCCCCAAGGACCTGAAATGACCCTGTGCCTTATTTGAAC | |
| | | TAACCAATCAGTTCGCTTCTCGCTTCTGTTCGCGCGCTTCTGC | |
| | | TCCCCGAGCTCTATATAAGCAGAGCTCGTTTAGTGAACCGTCA | |
| | | GATCGCCTGGAGACGCCATCCACGCTGTTTTGACTTCCATAGA | |
| | | AGGATCTCGAGGCCACCATGCCTCTGGGCCTGCTGTGGCTGGG | |
| | | CCTGGCCCTGCTGGGCGCCCTGCACGCCCAGGCCGAGATGTGG | |
| | | CACGAGGGCCTGGAGGAGGCCAGCAGGCTGTATTTTGGCGAGC | |
| | | GCAACGTGAAGGGCATGTTCGAGGTGCTGGAGCCTCTGCACGC | |
| | | CATGATGGAGAGAGGCCCACAGACCCTGAAGGAGACATCCTTT | |
| | | AACCAGGCCTATGGACGGGACCTGATGGAGGCACAGGAGTGGT | |
| | | GCAGAAAGTACATGAAGTCTGGCAATGTGAAGGACCTGACGCA | |
| | | GGCCTGGGATCTGTACTATCACGTGTTTCGGAGAATCTCCAAG | |
| | | GGCAAAGACACGATTCCGTGGCTTGGGCATCTGCTCGTTGGGC | |
| | | TGAGTGGTGCGTTTGGTTTCATCATCTTGGTCTATCTCTTGAT | |
| | | CAATTGCAGAAATACAGGCCCTTGGCTGAAAAAAGTGCTCAAG | |
| | | TGTAATACCCCCGACCCAAGCAAGTTCTTCTCCCAGCTTTCTT | |
| | | CAGAGCATGGAGGCGATGTGCAGAAATGGCTCTCTTCACCTTT | |
| | | TCCCTCCTCAAGTTTCTCCCCGGGAGGGCTGGCGCCCGAGATT | |
| | | TCACCTCTTGAGGTACTTGAACGAGACAAGGTTACCCAACTTC | |
| | | TCCTTCAACAGGATAAGGTACCCGAACCTGCGAGCCTTAGCTC | |
| | | CAACCACTCTCTTACGAGCTGCTTCACCAATCAGGGATACTTC | |
| | | TTTTTCCACCTTCCCGATGCGCTGGAAATCGAAGCATGTCAAG | |
| | | TTTACTTTACCTATGATCCATATAGCGAGGAAGATCCCGACGA | |
| | | AGGAGTCGCCGGTGCGCCCACGGGTTCCTCACCCCAACCTCTC | |
| | | CAGCCTCTCTCAGGAGAAGATGATGCTTATTGCACTTTTCCCA | |
| | | GTAGAGACGATCTCCTCCTCTTTTCTCCATCTCTTTTGGGGGG | |
| | | ACCTTCCCCCCCTTCTACGGCACCTGGCGGGTCTGGTGCTGGC | |
| | | GAGGAGCGGATGCCGCCGTCCCTCCAGGAGCGAGTACCACGAG | |
| | | ATTGGGATCCCCAGCCACTTGGACCACCAACTCCAGGCGTACC | |
| | | TGACCTTGTCGATTTTCAACCTCCCCCTGAATTGGTGCTGCGA | |
| | | GAGGCTGGGGAGGAAGTTCCGGACGCTGGGCCGAGGGAGGGCG | |
| | | TGTCCTTTCCATGGAGTAGGCCTCCAGGTCAAGGCGAGTTTAG | |
| | | GGCTCTCAACGCGCGGCTGCCGTTGAATACAGACGCTTATCTC | |
| | | TCACTGCAGGAACTGCAAGGTCAGGACCCAACACATCTTGTAG | |
| | | GATCCGGCGCCACCAACTTTAGTCTGCTCAAGCAAGCCGGGGA | |
| | | CGTCGAGGAAAATCCTGGGCCAGAAATGTGGCACGAAGGACTC | |
| | | GAGGAAGCCAGTCGGCTGTATTTTGGCGAGCGGAATGTGAAAG | |
| | | GGATGTTTGAAGTGCTCGAGCCTCTCCACGCTATGATGGAACG | |
| | | GGGACCCCAGACTCTCAAAGAAACCAGCTTTAATCAGGCTTAC | |
| | | GGACGCGACCTCATGGAAGCTCAAGAATGGTGTAGAAAGTATA | |
| | | TGAAGAGTGGCAACGTGAAAGATCTGACACAAGCCTGGGATCT | |
| | | CTATTATCACGTGTTCAGACGCATCAGCAAAGGCAGCGGCGCC | |
| | | ACAAATTTCTCCCTGCTGAAACAGGCCGGCGACGTGGAAGAGA | |
| | | ATCCCGGACCTATGCCCAATCCTAGACCTGGCAAGCCCAGCGC | |
| | | TCCTTCTCTTGCTCTTGGACCTTCTCCTGGTGCA | |
| | Full-length | ACTTGCCAGGACTGTTACAATAGCCTCCTCACTAGCCCCACTC | 151 |
| | donor | ACAGCAGCCAGATGAATCTTTTGAGTCCATGCCTAGTCACTGG | |
| | template | GGCAAAATAGGACTCCGAGGAGAAAGTCCGAGACCAGCTCCGG | |
| | | CAAGATGAGCAAACACAGCCTGTGCAGGGTGCAGGGAGGGCTA | |
| | | GAGGCCTGAGGCTTGAAACAGCTCTCAAGTGGAGGGGGAAACA | |
| | | ACCATTGCCCTCATAGAGGACACATCCACACCAGGGCTGTGCT | |
| | | AGCGTGGGCAGGCAAGCCAGGTGCTGGACCTCTGCACGTGGGG | |
| | | CATGTGTGGGTATGTACATGTACCTGTGTTCTTGGTGTGTGTG | |
| | | TGTGTGTGTGTGTGTGTGTGTGTCTAGAGCTGGGGTGCAACTA | |
| | | TGGGGCCCCTCGGGACATGTCCCAGCCAATGCCTGCTTTGACC | |
| | | AGAGGAGTGTCCACGTGGCTCAGGTGGTCGAGTATCTCATACC | |
| | | GCCCTAGCACACGTGTGACTCCTTTCCCCTATTGTCTACGCAG | |
| | | CCTGCCCTTGGACAAGGACCCGATGCCCAACCCCAGGCCTGGC | |
| | | AAGCCCTCGGCCCCTTCCTTGGCCCTTGGCCCATCCCCAGGAT | |
| | | GAACAGAGAAACAGGAGAATATGGGCCAAACAGGATATCTGTG | |
| | | GTAAGCAGTTCCTGCCCCGGCTCAGGGCCAAGAACAGTTGGAA | |
| | | CAGCAGAATATGGGCCAAACAGGATATCTGTGGTAAGCAGTTC | |
| | | CTGCCCCGGCTCAGGGCCAAGAACAGATGGTCCCCAGATGCGG | |
| | | TCCCGCCCTCAGCAGTTTCTAGAGAACCATCAGATGTTTCCAG | |
| | | GGTGCCCCAAGGACCTGAAATGACCCTGTGCCTTATTTGAACT | |
| | | AACCAATCAGTTCGCTTCTCGCTTCTGTTCGCGCGCTTCTGCT | |
| | | CCCCGAGCTCTATATAAGCAGAGCTCGTTTAGTGAACCGTCAG | |
| | | ATCGCCTGGAGACGCCATCCACGCTGTTTTGACTTCCATAGAA | |
| | | GGATCTCGAGGCCACCATGCCTCTGGGCCTGCTGTGGCTGGGC | |
| | | CTGGCCCTGCTGGGCGCCCTGCACGCCCAGGCCGAGATGTGGC | |
| | | ACGAGGGCCTGGAGGAGGCCAGCAGGCTGTATTTTGGCGAGCG | |
| | | CAACGTGAAGGGCATGTTCGAGGTGCTGGAGCCTCTGCACGCC | |
| | | ATGATGGAGAGAGGCCCACAGACCCTGAAGGAGACATCCTTTA | |
| | | ACCAGGCCTATGGACGGGACCTGATGGAGGCACAGGAGTGGTG | |
| | | CAGAAAGTACATGAAGTCTGGCAATGTGAAGGACCTGACGCAG | |
| | | GCCTGGGATCTGTACTATCACGTGTTTCGGAGAATCTCCAAGG | |
| | | GCAAAGACACGATTCCGTGGCTTGGGCATCTGCTCGTTGGGCT | |
| | | GAGTGGTGCGTTTGGTTTCATCATCTTGGTCTATCTCTTGATC | |
| | | AATTGCAGAAATACAGGCCCTTGGCTGAAAAAAGTGCTCAAGT | |
| | | GTAATACCCCCGACCCAAGCAAGTTCTTCTCCCAGCTTTCTTC | |
| | | AGAGCATGGAGGCGATGTGCAGAAATGGCTCTCTTCACCTTTT | |
| | | CCCTCCTCAAGTTTCTCCCCGGGAGGGCTGGCGCCCGAGATTT | |
| | | CACCTCTTGAGGTACTTGAACGAGACAAGGTTACCCAACTTCT | |
| | | CCTTCAACAGGATAAGGTACCCGAACCTGCGAGCCTTAGCTCC | |
| | | AACCACTCTCTTACGAGCTGCTTCACCAATCAGGGATACTTCT | |
| | | TTTTCCACCTTCCCGATGCGCTGGAAATCGAAGCATGTCAAGT | |
| | | TTACTTTACCTATGATCCATATAGCGAGGAAGATCCCGACGAA | |
| | | GGAGTCGCCGGTGCGCCCACGGGTTCCTCACCCCAACCTCTCC | |
| | | AGCCTCTCTCAGGAGAAGATGATGCTTATTGCACTTTTCCCAG | |
| | | TAGAGACGATCTCCTCCTCTTTTCTCCATCTCTTTTGGGGGGA | |
| | | CCTTCCCCCCCTTCTACGGCACCTGGCGGGTCTGGTGCTGGCG | |
| | | AGGAGCGGATGCCGCCGTCCCTCCAGGAGCGAGTACCACGAGA | |
| | | TTGGGATCCCCAGCCACTTGGACCACCAACTCCAGGCGTACCT | |
| | | GACCTTGTCGATTTTCAACCTCCCCCTGAATTGGTGCTGCGAG | |
| | | AGGCTGGGGAGGAAGTTCCGGACGCTGGGCCGAGGGAGGGCGT | |
| | | GTCCTTTCCATGGAGTAGGCCTCCAGGTCAAGGCGAGTTTAGG | |
| | | GCTCTCAACGCGCGGCTGCCGTTGAATACAGACGCTTATCTCT | |
| | | CACTGCAGGAACTGCAAGGTCAGGACCCAACACATCTTGTAGG | |
| | | ATCCGGCGCCACCAACTTTAGTCTGCTCAAGCAAGCCGGGGAC | |
| | | GTCGAGGAAAATCCTGGGCCAGAAATGTGGCACGAAGGACTCG | |
| | | AGGAAGCCAGTCGGCTGTATTTTGGCGAGCGGAATGTGAAAGG | |
| | | GATGTTTGAAGTGCTCGAGCCTCTCCACGCTATGATGGAACGG | |
| | | GGACCCCAGACTCTCAAAGAAACCAGCTTTAATCAGGCTTACG | |
| | | GACGCGACCTCATGGAAGCTCAAGAATGGTGTAGAAAGTATAT | |
| | | GAAGAGTGGCAACGTGAAAGATCTGACACAAGCCTGGGATCTC | |
| | | TATTATCACGTGTTCAGACGCATCAGCAAAGGCAGCGGCGCCA | |
| | | CAAATTTCTCCCTGCTGAAACAGGCCGGCGACGTGGAAGAGAA | |
| | | TCCCGGACCTATGCCCAATCCTAGACCTGGCAAGCCCAGCGCT | |
| | | CCTTCTCTTGCTCTTGGACCTTCTCCTGGTGCATCGCCCAGCT | |
| | | GGAGGGCTGCACCCAAAGCCTCAGACCTGCTGGGGGCCCGGGG | |
| | | CCCAGGGGGAACCTTCCAGGGCCGAGATCTTCGAGGCGGGGCC | |
| | | CATGCCTCCTCTTCTTCCTTGAACCCCATGCCACCATCGCAGC | |
| | | TGCAGGTGAGGCCCTGGGCCCAGGATGGGGCAGGCAGGGTGGG | |
| | | GTACCTGGACCTACAGGTGCCGACCTTTACTGTGGCACTGGGC | |
| | | GGGAGGGGGGCTGGCTGGGGCACAGGAAGTGGTTTCTGGGTCC | |
| | | CAGGCAAGTCTGTGACTTATGCAGATGTTGCAGGGCCAAGAAA | |
| | | ATCCCCACCTGCCAGGCCTCAGAGATTGGAGGCTCTCCCCGAC | |
| | | CTCCCAATCCCTGTCTCAGGAGAGGAGGAGGCCGTATTGTAGT | |
| | | CCCATGAGCATAGCTATGTGTCCCCATCCCCATGTGACAAGAG | |
| | | AAGAGGACTGGGGCCAAGTAGGTGAGGTGACAGGGCTGAGGCC | |
| | | AGCTCTGCAACTTATTAGCTGTTTGATCTTTAAAAAGTTACTC | |
| | | GATCTCCATGAGCCTCAGTTTCCATACGTGTAAAAGGGGGATG | |
| | | ATCATAGCATCTACCATGTGGGCTTGCAGTG | |
| FoxP3_v2 | 5′ Homology | CCCACTACATCCAAGCTGCTAGCACTGCTCCTGATCCAGCTTC | 152 |
| | Arm | AGATTAAGTCTCAGAATCTACCCACTTCTCGCCTTCTCCACTG | |
| | | CCACCAGCCCATTCTGTGCCAGCATCATCACTTGCCAGGACTG | |
| | | TTACAATAGCCTCCTCACTAGCCCCACTCACAGCAGCCAGATG | |
| | | AATCTTTTGAGTCCATGCCTAGTCACTGGGGCAAAATAGGACT | |
| | | CCGAGGAGAAAGTCCGAGACCAGCTCCGGCAAGATGAGCAAAC | |
| | | ACAGCCTGTGCAGGGTGCAGGGAGGGCTAGAGGCCTGAGGCTT | |
| | | GAAACAGCTCTCAAGTGGAGGGGGAAACAACCATTGCCCTCAT | |
| | | AGAGGACACATCCACACCAGGGCTGTGCTAGCGTGGGCAGGCA | |
| | | AGCCAGGTGCTGGACCTCTGCACGTGGGGCATGTGTGGGTATG | |
| | | TACATGTACCTGTGTTCTTGGTGTGTGTGTGTGTGTGTGTGTG | |
| | | TGTGTGTGTCTAGAGCTGGGGTGCAACTATGGGGCCCCTCGGG | |
| | | ACATGTCCCAGCCAATGCCTGCTTTGACCAGAGGAGTGTCCAC | |
| | | GTGGCTCAGGTGGTCGAGTATCTCATACCGCCCTAGCACACGT | |
| | | GTGACTCCTTTCCCCTATTGTCTACGCAGCCTGCCCTTGGACA | |
| | | AGGAC | |
| | Intervening | ACGATGT | |
| | sequence | | |
| | MND Promoter | GAACAGAGAAACAGGAGAATATGGGCCAAACAGGATATCTGTG | 153 |
| | | GTAAGCAGTTCCTGCCCCGGCTCAGGGCCAAGAACAGTTGGAA | |
| | | CAGCAGAATATGGGCCAAACAGGATATCTGTGGTAAGCAGTTC | |
| | | CTGCCCCGGCTCAGGGCCAAGAACAGATGGTCCCCAGATGCGG | |
| | | TCCCGCCCTCAGCAGTTTCTAGAGAACCATCAGATGTTTCCAG | |
| | | GGTGCCCCAAGGACCTGAAATGACCCTGTGCCTTATTTGAACT | |
| | | AACCAATCAGTTCGCTTCTCGCTTCTGTTCGCGCGCTTCTGCT | |
| | | CCCCGAGCTCTATATAAGCAGAGCTCGTTTAGTGAACCGTCAG | |
| | | ATC | |
| | Intervening | GCCTGGAGACGCCATCCACGCTGTTTTGACTTCCATAGAAGGA | 154 |
| | Sequence | TCTCGAGGCCACCATGCCTCTGGGCCTGCTGTGGCTGGGCCTG | |
| | (LCN2 signal | GCCCTGCTGGGCGCCCTGCACGCCCAGGCC | |
| | peptide of | | |
| | FRB-IL2Rb | | |
| | underlined) | | |
| | FRB-IL2Rb | GAGATGTGGCACGAGGGCCTGGAGGAGGCCAGCAGGCTGTATT | 155 |
| | | TTGGCGAGCGCAACGTGAAGGGCATGTTCGAGGTGCTGGAGCC | |
| | | TCTGCACGCCATGATGGAGAGAGGCCCACAGACCCTGAAGGAG | |
| | | ACATCCTTTAACCAGGCCTATGGACGGGACCTGATGGAGGCAC | |
| | | AGGAGTGGTGCAGAAAGTACATGAAGTCTGGCAATGTGAAGGA | |
| | | CCTGACGCAGGCCTGGGATCTGTACTATCACGTGTTTCGGAGA | |
| | | ATCTCCAAGGGCAAAGACACGATTCCGTGGCTTGGGCATCTGC | |
| | | TCGTTGGGCTGAGTGGTGCGTTTGGTTTCATCATCTTGGTCTA | |
| | | TCTCTTGATCAATTGCAGAAATACAGGCCCTTGGCTGAAAAAA | |
| | | GTGCTCAAGTGTAATACCCCCGACCCAAGCAAGTTCTTCTCCC | |
| | | AGCTTTCTTCAGAGCATGGAGGCGATGTGCAGAAATGGCTCTC | |
| | | TTCACCTTTTCCCTCCTCAAGTTTCTCCCCGGGAGGGCTGGCG | |
| | | CCCGAGATTTCACCTCTTGAGGTACTTGAACGAGACAAGGTTA | |
| | | CCCAACTTCTCCTTCAACAGGATAAGGTACCCGAACCTGCGAG | |
| | | CCTTAGCTCCAACCACTCTCTTACGAGCTGCTTCACCAATCAG | |
| | | GGATACTTCTTTTTCCACCTTCCCGATGCGCTGGAAATCGAAG | |
| | | CATGTCAAGTTTACTTTACCTATGATCCATATAGCGAGGAAGA | |
| | | TCCCGACGAAGGAGTCGCCGGTGCGCCCACGGGTTCCTCACCC | |
| | | CAACCTCTCCAGCCTCTCTCAGGAGAAGATGATGCTTATTGCA | |
| | | CTTTTCCCAGTAGAGACGATCTCCTCCTCTTTTCTCCATCTCT | |
| | | TTTGGGGGGACCTTCCCCCCCTTCTACGGCACCTGGCGGGTCT | |
| | | GGTGCTGGCGAGGAGCGGATGCCGCCGTCCCTCCAGGAGCGAG | |
| | | TACCACGAGATTGGGATCCCCAGCCACTTGGACCACCAACTCC | |
| | | AGGCGTACCTGACCTTGTCGATTTTCAACCTCCCCCTGAATTG | |
| | | GTGCTGCGAGAGGCTGGGGAGGAAGTTCCGGACGCTGGGCCGA | |
| | | GGGAGGGCGTGTCCTTTCCATGGAGTAGGCCTCCAGGTCAAGG | |
| | | CGAGTTTAGGGCTCTCAACGCGCGGCTGCCGTTGAATACAGAC | |
| | | GCTTATCTCTCACTGCAGGAACTGCAAGGTCAGGACCCAACAC | |
| | | ATCTTGTA | |
| | GSG linker | GGATCCGGC | |
| | P2A | GCCACCAACTTTAGTCTGCTCAAGCAAGCCGGGGACGTCGAGG | 156 |
| | | AAAATCCTGGGCCA | |
| | Cytosolic | GAAATGTGGCACGAAGGACTCGAGGAAGCCAGTCGGCTGTATT | 157 |
| | FRB | TTGGCGAGCGGAATGTGAAAGGGATGTTTGAAGTGCTCGAGCC | |
| | | TCTCCACGCTATGATGGAACGGGGACCCCAGACTCTCAAAGAA | |
| | | ACCAGCTTTAATCAGGCTTACGGACGCGACCTCATGGAAGCTC | |
| | | AAGAATGGTGTAGAAAGTATATGAAGAGTGGCAACGTGAAAGA | |
| | | TCTGACACAAGCCTGGGATCTCTATTATCACGTGTTCAGACGC | |
| | | ATCAGCAAA | |
| | GSG linker | GGCAGCGGC | |
| | Second P2A | GCCACAAATTTCTCCCTGCTGAAACAGGCCGGCGACGTGGAAG | 158 |
| | | AGAATCCCGGACCT | |
| | FoxP3 | ATGCCAAATCCT | 159 |
| | portion | | |
| | 3′ Homology | AGGCCTGGCAAGCCCTCGGCCCCTTCCTTGGCCCTTGGCCCAT | 160 |
| | Arm | CCCCAGGAGCCTCGCCCAGCTGGAGGGCTGCACCCAAAGCCTC | |
| | | AGACCTGCTGGGGGCCCGGGGCCCAGGGGGAACCTTCCAGGGC | |
| | | CGAGATCTTCGAGGCGGGGCCCATGCCTCCTCTTCTTCCTTGA | |
| | | ACCCCATGCCACCATCGCAGCTGCAGGTGAGGCCCTGGGCCCA | |
| | | GGATGGGGCAGGCAGGGTGGGGTACCTGGACCTACAGGTGCCG | |
| | | ACCTTTACTGTGGCACTGGGCGGGAGGGGGGCTGGCTGGGGCA | |
| | | CAGGAAGTGGTTTCTGGGTCCCAGGCAAGTCTGTGACTTATGC | |
| | | AGATGTTGCAGGGCCAAGAAAATCCCCACCTGCCAGGCCTCAG | |
| | | AGATTGGAGGCTCTCCCCGACCTCCCAATCCCTGTCTCAGGAG | |
| | | AGGAGGAGGCCGTATTGTAGTCCCATGAGCATAGCTATGTGTC | |
| | | CCCATCCCCATGTGACAAGAGAAGAGGACTGGGGCCAAGTAGG | |
| | | TGAGGTGACAGGGCTGAGGCCAGCTCTGCAACTTATTAGCTGT | |
| | | TTGATCTTTAAAAAGTTACTCGATCTCCATGAGCCTCAGTTTC | |
| | | CATACGTGTAAAAGGGGGATGATCATAGCATCTACCATGTGGG | |
| | | CTTGC | |
| | Between | ACGATGTGAACAGAGAAACAGGAGAATATGGGCCAAACAGGAT | 161 |
| | Homology | ATCTGTGGTAAGCAGTTCCTGCCCCGGCTCAGGGCCAAGAACA | |
| | Arms | GTTGGAACAGCAGAATATGGGCCAAACAGGATATCTGTGGTAA | |
| | | GCAGTTCCTGCCCCGGCTCAGGGCCAAGAACAGATGGTCCCCA | |
| | | GATGCGGTCCCGCCCTCAGCAGTTTCTAGAGAACCATCAGATG | |
| | | TTTCCAGGGTGCCCCAAGGACCTGAAATGACCCTGTGCCTTAT | |
| | | TTGAACTAACCAATCAGTTCGCTTCTCGCTTCTGTTCGCGCGC | |
| | | TTCTGCTCCCCGAGCTCTATATAAGCAGAGCTCGTTTAGTGAA | |
| | | CCGTCAGATCGCCTGGAGACGCCATCCACGCTGTTTTGACTTC | |
| | | CATAGAAGGATCTCGAGGCCACCATGCCTCTGGGCCTGCTGTG | |
| | | GCTGGGCCTGGCCCTGCTGGGCGCCCTGCACGCCCAGGCCGAG | |
| | | ATGTGGCACGAGGGCCTGGAGGAGGCCAGCAGGCTGTATTTTG | |
| | | GCGAGCGCAACGTGAAGGGCATGTTCGAGGTGCTGGAGCCTCT | |
| | | GCACGCCATGATGGAGAGAGGCCCACAGACCCTGAAGGAGACA | |
| | | TCCTTTAACCAGGCCTATGGACGGGACCTGATGGAGGCACAGG | |
| | | AGTGGTGCAGAAAGTACATGAAGTCTGGCAATGTGAAGGACCT | |
| | | GACGCAGGCCTGGGATCTGTACTATCACGTGTTTCGGAGAATC | |
| | | TCCAAGGGCAAAGACACGATTCCGTGGCTTGGGCATCTGCTCG | |
| | | TTGGGCTGAGTGGTGCGTTTGGTTTCATCATCTTGGTCTATCT | |
| | | CTTGATCAATTGCAGAAATACAGGCCCTTGGCTGAAAAAAGTG | |
| | | CTCAAGTGTAATACCCCCGACCCAAGCAAGTTCTTCTCCCAGC | |
| | | TTTCTTCAGAGCATGGAGGCGATGTGCAGAAATGGCTCTCTTC | |
| | | ACCTTTTCCCTCCTCAAGTTTCTCCCCGGGAGGGCTGGCGCCC | |
| | | GAGATTTCACCTCTTGAGGTACTTGAACGAGACAAGGTTACCC | |
| | | AACTTCTCCTTCAACAGGATAAGGTACCCGAACCTGCGAGCCT | |
| | | TAGCTCCAACCACTCTCTTACGAGCTGCTTCACCAATCAGGGA | |
| | | TACTTCTTTTTCCACCTTCCCGATGCGCTGGAAATCGAAGCAT | |
| | | GTCAAGTTTACTTTACCTATGATCCATATAGCGAGGAAGATCC | |
| | | CGACGAAGGAGTCGCCGGTGCGCCCACGGGTTCCTCACCCCAA | |
| | | CCTCTCCAGCCTCTCTCAGGAGAAGATGATGCTTATTGCACTT | |
| | | TTCCCAGTAGAGACGATCTCCTCCTCTTTTCTCCATCTCTTTT | |
| | | GGGGGGACCTTCCCCCCCTTCTACGGCACCTGGCGGGTCTGGT | |
| | | GCTGGCGAGGAGCGGATGCCGCCGTCCCTCCAGGAGCGAGTAC | |
| | | CACGAGATTGGGATCCCCAGCCACTTGGACCACCAACTCCAGG | |
| | | CGTACCTGACCTTGTCGATTTTCAACCTCCCCCTGAATTGGTG | |
| | | CTGCGAGAGGCTGGGGAGGAAGTTCCGGACGCTGGGCCGAGGG | |
| | | AGGGCGTGTCCTTTCCATGGAGTAGGCCTCCAGGTCAAGGCGA | |
| | | GTTTAGGGCTCTCAACGCGCGGCTGCCGTTGAATACAGACGCT | |
| | | TATCTCTCACTGCAGGAACTGCAAGGTCAGGACCCAACACATC | |
| | | TTGTAGGATCCGGCGCCACCAACTTTAGTCTGCTCAAGCAAGC | |
| | | CGGGGACGTCGAGGAAAATCCTGGGCCAGAAATGTGGCACGAA | |
| | | GGACTCGAGGAAGCCAGTCGGCTGTATTTTGGCGAGCGGAATG | |
| | | TGAAAGGGATGTTTGAAGTGCTCGAGCCTCTCCACGCTATGAT | |
| | | GGAACGGGGACCCCAGACTCTCAAAGAAACCAGCTTTAATCAG | |
| | | GCTTACGGACGCGACCTCATGGAAGCTCAAGAATGGTGTAGAA | |
| | | AGTATATGAAGAGTGGCAACGTGAAAGATCTGACACAAGCCTG | |
| | | GGATCTCTATTATCACGTGTTCAGACGCATCAGCAAAGGCAGC | |
| | | GGCGCCACAAATTTCTCCCTGCTGAAACAGGCCGGCGACGTGG | |
| | | AAGAGAATCCCGGACCTATGCCAAATCCT | |
| | Full-length | CCCACTACATCCAAGCTGCTAGCACTGCTCCTGATCCAGCTTC | 162 |
| | donor | AGATTAAGTCTCAGAATCTACCCACTTCTCGCCTTCTCCACTG | |
| | template | CCACCAGCCCATTCTGTGCCAGCATCATCACTTGCCAGGACTG | |
| | | TTACAATAGCCTCCTCACTAGCCCCACTCACAGCAGCCAGATG | |
| | | AATCTTTTGAGTCCATGCCTAGTCACTGGGGCAAAATAGGACT | |
| | | CCGAGGAGAAAGTCCGAGACCAGCTCCGGCAAGATGAGCAAAC | |
| | | ACAGCCTGTGCAGGGTGCAGGGAGGGCTAGAGGCCTGAGGCTT | |
| | | GAAACAGCTCTCAAGTGGAGGGGGAAACAACCATTGCCCTCAT | |
| | | AGAGGACACATCCACACCAGGGCTGTGCTAGCGTGGGCAGGCA | |
| | | AGCCAGGTGCTGGACCTCTGCACGTGGGGCATGTGTGGGTATG | |
| | | TACATGTACCTGTGTTCTTGGTGTGTGTGTGTGTGTGTGTGTG | |
| | | TGTGTGTGTCTAGAGCTGGGGTGCAACTATGGGGCCCCTCGGG | |
| | | ACATGTCCCAGCCAATGCCTGCTTTGACCAGAGGAGTGTCCAC | |
| | | GTGGCTCAGGTGGTCGAGTATCTCATACCGCCCTAGCACACGT | |
| | | GTGACTCCTTTCCCCTATTGTCTACGCAGCCTGCCCTTGGACA | |
| | | AGGACACGATGTGAACAGAGAAACAGGAGAATATGGGCCAAAC | |
| | | AGGATATCTGTGGTAAGCAGTTCCTGCCCCGGCTCAGGGCCAA | |
| | | GAACAGTTGGAACAGCAGAATATGGGCCAAACAGGATATCTGT | |
| | | GGTAAGCAGTTCCTGCCCCGGCTCAGGGCCAAGAACAGATGGT | |
| | | CCCCAGATGCGGTCCCGCCCTCAGCAGTTTCTAGAGAACCATC | |
| | | AGATGTTTCCAGGGTGCCCCAAGGACCTGAAATGACCCTGTGC | |
| | | CTTATTTGAACTAACCAATCAGTTCGCTTCTCGCTTCTGTTCG | |
| | | CGCGCTTCTGCTCCCCGAGCTCTATATAAGCAGAGCTCGTTTA | |
| | | GTGAACCGTCAGATCGCCTGGAGACGCCATCCACGCTGTTTTG | |
| | | ACTTCCATAGAAGGATCTCGAGGCCACCATGCCTCTGGGCCTG | |
| | | CTGTGGCTGGGCCTGGCCCTGCTGGGCGCCCTGCACGCCCAGG | |
| | | CCGAGATGTGGCACGAGGGCCTGGAGGAGGCCAGCAGGCTGTA | |
| | | TTTTGGCGAGCGCAACGTGAAGGGCATGTTCGAGGTGCTGGAG | |
| | | CCTCTGCACGCCATGATGGAGAGAGGCCCACAGACCCTGAAGG | |
| | | AGACATCCTTTAACCAGGCCTATGGACGGGACCTGATGGAGGC | |
| | | ACAGGAGTGGTGCAGAAAGTACATGAAGTCTGGCAATGTGAAG | |
| | | GACCTGACGCAGGCCTGGGATCTGTACTATCACGTGTTTCGGA | |
| | | GAATCTCCAAGGGCAAAGACACGATTCCGTGGCTTGGGCATCT | |
| | | GCTCGTTGGGCTGAGTGGTGCGTTTGGTTTCATCATCTTGGTC | |
| | | TATCTCTTGATCAATTGCAGAAATACAGGCCCTTGGCTGAAAA | |
| | | AAGTGCTCAAGTGTAATACCCCCGACCCAAGCAAGTTCTTCTC | |
| | | CCAGCTTTCTTCAGAGCATGGAGGCGATGTGCAGAAATGGCTC | |
| | | TCTTCACCTTTTCCCTCCTCAAGTTTCTCCCCGGGAGGGCTGG | |
| | | CGCCCGAGATTTCACCTCTTGAGGTACTTGAACGAGACAAGGT | |
| | | TACCCAACTTCTCCTTCAACAGGATAAGGTACCCGAACCTGCG | |
| | | AGCCTTAGCTCCAACCACTCTCTTACGAGCTGCTTCACCAATC | |
| | | AGGGATACTTCTTTTTCCACCTTCCCGATGCGCTGGAAATCGA | |
| | | AGCATGTCAAGTTTACTTTACCTATGATCCATATAGCGAGGAA | |
| | | GATCCCGACGAAGGAGTCGCCGGTGCGCCCACGGGTTCCTCAC | |
| | | CCCAACCTCTCCAGCCTCTCTCAGGAGAAGATGATGCTTATTG | |
| | | CACTTTTCCCAGTAGAGACGATCTCCTCCTCTTTTCTCCATCT | |
| | | CTTTTGGGGGGACCTTCCCCCCCTTCTACGGCACCTGGCGGGT | |
| | | CTGGTGCTGGCGAGGAGCGGATGCCGCCGTCCCTCCAGGAGCG | |
| | | AGTACCACGAGATTGGGATCCCCAGCCACTTGGACCACCAACT | |
| | | CCAGGCGTACCTGACCTTGTCGATTTTCAACCTCCCCCTGAAT | |
| | | TGGTGCTGCGAGAGGCTGGGGAGGAAGTTCCGGACGCTGGGCC | |
| | | GAGGGAGGGCGTGTCCTTTCCATGGAGTAGGCCTCCAGGTCAA | |
| | | GGCGAGTTTAGGGCTCTCAACGCGCGGCTGCCGTTGAATACAG | |
| | | ACGCTTATCTCTCACTGCAGGAACTGCAAGGTCAGGACCCAAC | |
| | | ACATCTTGTAGGATCCGGCGCCACCAACTTTAGTCTGCTCAAG | |
| | | CAAGCCGGGGACGTCGAGGAAAATCCTGGGCCAGAAATGTGGC | |
| | | ACGAAGGACTCGAGGAAGCCAGTCGGCTGTATTTTGGCGAGCG | |
| | | GAATGTGAAAGGGATGTTTGAAGTGCTCGAGCCTCTCCACGCT | |
| | | ATGATGGAACGGGGACCCCAGACTCTCAAAGAAACCAGCTTTA | |
| | | ATCAGGCTTACGGACGCGACCTCATGGAAGCTCAAGAATGGTG | |
| | | TAGAAAGTATATGAAGAGTGGCAACGTGAAAGATCTGACACAA | |
| | | GCCTGGGATCTCTATTATCACGTGTTCAGACGCATCAGCAAAG | |
| | | GCAGCGGCGCCACAAATTTCTCCCTGCTGAAACAGGCCGGCGA | |
| | | CGTGGAAGAGAATCCCGGACCTATGCCAAATCCTAGGCCTGGC | |
| | | AAGCCCTCGGCCCCTTCCTTGGCCCTTGGCCCATCCCCAGGAG | |
| | | CCTCGCCCAGCTGGAGGGCTGCACCCAAAGCCTCAGACCTGCT | |
| | | GGGGGCCCGGGGCCCAGGGGGAACCTTCCAGGGCCGAGATCTT | |
| | | CGAGGCGGGGCCCATGCCTCCTCTTCTTCCTTGAACCCCATGC | |
| | | CACCATCGCAGCTGCAGGTGAGGCCCTGGGCCCAGGATGGGGC | |
| | | AGGCAGGGTGGGGTACCTGGACCTACAGGTGCCGACCTTTACT | |
| | | GTGGCACTGGGCGGGAGGGGGGCTGGCTGGGGCACAGGAAGTG | |
| | | GTTTCTGGGTCCCAGGCAAGTCTGTGACTTATGCAGATGTTGC | |
| | | AGGGCCAAGAAAATCCCCACCTGCCAGGCCTCAGAGATTGGAG | |
| | | GCTCTCCCCGACCTCCCAATCCCTGTCTCAGGAGAGGAGGAGG | |
| | | CCGTATTGTAGTCCCATGAGCATAGCTATGTGTCCCCATCCCC | |
| | | ATGTGACAAGAGAAGAGGACTGGGGCCAAGTAGGTGAGGTGAC | |
| | | AGGGCTGAGGCCAGCTCTGCAACTTATTAGCTGTTTGATCTTT | |
| | | AAAAAGTTACTCGATCTCCATGAGCCTCAGTTTCCATACGTGT | |
| | | AAAAGGGGGATGATCATAGCATCTACCATGTGGGCTTGC | |
| FoxP3_v3 | 5′ Homology | ACTGCCACCAGCCCATTCTGTGCCAGCATCATCACTTGCCAGG | 163 |
| | Arm | ACTGTTACAATAGCCTCCTCACTAGCCCCACTCACAGCAGCCA | |
| | | GATGAATCTTTTGAGTCCATGCCTAGTCACTGGGGCAAAATAG | |
| | | GACTCCGAGGAGAAAGTCCGAGACCAGCTCCGGCAAGATGAGC | |
| | | AAACACAGCCTGTGCAGGGTGCAGGGAGGGCTAGAGGCCTGAG | |
| | | GCTTGAAACAGCTCTCAAGTGGAGGGGGAAACAACCATTGCCC | |
| | | TCATAGAGGACACATCCACACCAGGGCTGTGCTAGCGTGGGCA | |
| | | GGCAAGCCAGGTGCTGGACCTCTGCACGTGGGGCATGTGTGGG | |
| | | TATGTACATGTACCTGTGTTCTTGGTGTGTGTGTGTGTGTGTG | |
| | | TGTGTGTGTGTGTCTAGAGCTGGGGTGCAACTATGGGGCCCCT | |
| | | CGGGACATGTCCCAGCCAATGCCTGCTTTGACCAGAGGAGTGT | |
| | | CCACGTGGCTCAGGTGGTCGAGTATCTCATACCGCCCTAGCAC | |
| | | ACGTGTGACTCCTTTCCCCTATTGTCTACGCAGCCTGCCCTTG | |
| | | GACAAGGACCCGATGCCCAACCCCAGGCCTGGCAAGCCCTCGG | |
| | | CCCCTTCCTTGGCCCTTGGCCCATCCCCAGGAGCCTCGCCCAG | |
| | | CTGGAG | |
| | Intervening | AGCTGCAT | |
| | sequence | | |
| | MND Promoter | GAACAGAGAAACAGGAGAATATGGGCCAAACAGGATATCTGTG | 164 |
| | | GTAAGCAGTTCCTGCCCCGGCTCAGGGCCAAGAACAGTTGGAA | |
| | | CAGCAGAATATGGGCCAAACAGGATATCTGTGGTAAGCAGTTC | |
| | | CTGCCCCGGCTCAGGGCCAAGAACAGATGGTCCCCAGATGCGG | |
| | | TCCCGCCCTCAGCAGTTTCTAGAGAACCATCAGATGTTTCCAG | |
| | | GGTGCCCCAAGGACCTGAAATGACCCTGTGCCTTATTTGAACT | |
| | | AACCAATCAGTTCGCTTCTCGCTTCTGTTCGCGCGCTTCTGCT | |
| | | CCCCGAGCTCTATATAAGCAGAGCTCGTTTAGTGAACCGTCAG | |
| | | ATC | |
| | Intervening | GCCTGGAGACGCCATCCACGCTGTTTTGACTTCCATAGAAGGA | 165 |
| | Sequence | TCTCGAGGCCACCATGCCTCTGGGCCTGCTGTGGCTGGGCCTG | |
| | (LCN2 signal | GCCCTGCTGGGCGCCCTGCACGCCCAGGCC | |
| | peptide of | | |
| | FRB-IL2Rb | | |
| | underlined) | | |
| | FRB-IL2Rb | GAGATGTGGCACGAGGGCCTGGAGGAGGCCAGCAGGCTGTATT | 166 |
| | | TTGGCGAGCGCAACGTGAAGGGCATGTTCGAGGTGCTGGAGCC | |
| | | TCTGCACGCCATGATGGAGAGAGGCCCACAGACCCTGAAGGAG | |
| | | ACATCCTTTAACCAGGCCTATGGACGGGACCTGATGGAGGCAC | |
| | | AGGAGTGGTGCAGAAAGTACATGAAGTCTGGCAATGTGAAGGA | |
| | | CCTGACGCAGGCCTGGGATCTGTACTATCACGTGTTTCGGAGA | |
| | | ATCTCCAAGGGCAAAGACACGATTCCGTGGCTTGGGCATCTGC | |
| | | TCGTTGGGCTGAGTGGTGCGTTTGGTTTCATCATCTTGGTCTA | |
| | | TCTCTTGATCAATTGCAGAAATACAGGCCCTTGGCTGAAAAAA | |
| | | GTGCTCAAGTGTAATACCCCCGACCCAAGCAAGTTCTTCTCCC | |
| | | AGCTTTCTTCAGAGCATGGAGGCGATGTGCAGAAATGGCTCTC | |
| | | TTCACCTTTTCCCTCCTCAAGTTTCTCCCCGGGAGGGCTGGCG | |
| | | CCCGAGATTTCACCTCTTGAGGTACTTGAACGAGACAAGGTTA | |
| | | CCCAACTTCTCCTTCAACAGGATAAGGTACCCGAACCTGCGAG | |
| | | CCTTAGCTCCAACCACTCTCTTACGAGCTGCTTCACCAATCAG | |
| | | GGATACTTCTTTTTCCACCTTCCCGATGCGCTGGAAATCGAAG | |
| | | CATGTCAAGTTTACTTTACCTATGATCCATATAGCGAGGAAGA | |
| | | TCCCGACGAAGGAGTCGCCGGTGCGCCCACGGGTTCCTCACCC | |
| | | CAACCTCTCCAGCCTCTCTCAGGAGAAGATGATGCTTATTGCA | |
| | | CTTTTCCCAGTAGAGACGATCTCCTCCTCTTTTCTCCATCTCT | |
| | | TTTGGGGGGACCTTCCCCCCCTTCTACGGCACCTGGCGGGTCT | |
| | | GGTGCTGGCGAGGAGCGGATGCCGCCGTCCCTCCAGGAGCGAG | |
| | | TACCACGAGATTGGGATCCCCAGCCACTTGGACCACCAACTCC | |
| | | AGGCGTACCTGACCTTGTCGATTTTCAACCTCCCCCTGAATTG | |
| | | GTGCTGCGAGAGGCTGGGGAGGAAGTTCCGGACGCTGGGCCGA | |
| | | GGGAGGGCGTGTCCTTTCCATGGAGTAGGCCTCCAGGTCAAGG | |
| | | CGAGTTTAGGGCTCTCAACGCGCGGCTGCCGTTGAATACAGAC | |
| | | GCTTATCTCTCACTGCAGGAACTGCAAGGTCAGGACCCAACAC | |
| | | ATCTTGTA | |
| | GSG linker | GGATCCGGC | |
| | P2A | GCCACCAACTTTAGTCTGCTCAAGCAAGCCGGGGACGTCGAGG | 167 |
| | | AAAATCCTGGGCCA | |
| | Cytosolic | GAAATGTGGCACGAAGGACTCGAGGAAGCCAGTCGGCTGTATT | 168 |
| | FRB | TTGGCGAGCGGAATGTGAAAGGGATGTTTGAAGTGCTCGAGCC | |
| | | TCTCCACGCTATGATGGAACGGGGACCCCAGACTCTCAAAGAA | |
| | | ACCAGCTTTAATCAGGCTTACGGACGCGACCTCATGGAAGCTC | |
| | | AAGAATGGTGTAGAAAGTATATGAAGAGTGGCAACGTGAAAGA | |
| | | TCTGACACAAGCCTGGGATCTCTATTATCACGTGTTCAGACGC | |
| | | ATCAGCAAA | |
| | GSG linker | GGCAGCGGC | |
| | Second P2A | GCCACAAATTTCTCCCTGCTGAAACAGGCCGGCGACGTGGAAG | 169 |
| | | AGAATCCCGGACCT | |
| | FoxP3 | ATGCCCAATCCTAGACCTGGCAAGCCCAGCGCTCCTTCTCTTG | 170 |
| | portion | CTCTTGGACCTTCTCCTGGTGCAAGTCCATCTTGGCGAGCAGC | |
| | | TCCAAAGGCT | |
| | 3′ Homology | TCAGACCTGCTGGGGGCCCGGGGCCCAGGGGGAACCTTCCAGG | 171 |
| | Arm | GCCGAGATCTTCGAGGCGGGGCCCATGCCTCCTCTTCTTCCTT | |
| | | GAACCCCATGCCACCATCGCAGCTGCAGGTGAGGCCCTGGGCC | |
| | | CAGGATGGGGCAGGCAGGGTGGGGTACCTGGACCTACAGGTGC | |
| | | CGACCTTTACTGTGGCACTGGGCGGGAGGGGGGCTGGCTGGGG | |
| | | CACAGGAAGTGGTTTCTGGGTCCCAGGCAAGTCTGTGACTTAT | |
| | | GCAGATGTTGCAGGGCCAAGAAAATCCCCACCTGCCAGGCCTC | |
| | | AGAGATTGGAGGCTCTCCCCGACCTCCCAATCCCTGTCTCAGG | |
| | | AGAGGAGGAGGCCGTATTGTAGTCCCATGAGCATAGCTATGTG | |
| | | TCCCCATCCCCATGTGACAAGAGAAGAGGACTGGGGCCAAGTA | |
| | | GGTGAGGTGACAGGGCTGAGGCCAGCTCTGCAACTTATTAGCT | |
| | | GTTTGATCTTTAAAAAGTTACTCGATCTCCATGAGCCTCAGTT | |
| | | TCCATACGTGTAAAAGGGGGATGATCATAGCATCTACCATGTG | |
| | | GGCTTGCAGTGCAGAGTATTTGAATTAGACACAGAACAGTGAG | |
| | | GATCAGGATGGCCTCTCACCCACCTGCCTTTCTGCCCAGCTGC | |
| | | CCACA | |
| | Between | AGCTGCATGAACAGAGAAACAGGAGAATATGGGCCAAACAGGA | 172 |
| | Homology | TATCTGTGGTAAGCAGTTCCTGCCCCGGCTCAGGGCCAAGAAC | |
| | Arms | AGTTGGAACAGCAGAATATGGGCCAAACAGGATATCTGTGGTA | |
| | | AGCAGTTCCTGCCCCGGCTCAGGGCCAAGAACAGATGGTCCCC | |
| | | AGATGCGGTCCCGCCCTCAGCAGTTTCTAGAGAACCATCAGAT | |
| | | GTTTCCAGGGTGCCCCAAGGACCTGAAATGACCCTGTGCCTTA | |
| | | TTTGAACTAACCAATCAGTTCGCTTCTCGCTTCTGTTCGCGCG | |
| | | CTTCTGCTCCCCGAGCTCTATATAAGCAGAGCTCGTTTAGTGA | |
| | | ACCGTCAGATCGCCTGGAGACGCCATCCACGCTGTTTTGACTT | |
| | | CCATAGAAGGATCTCGAGGCCACCATGCCTCTGGGCCTGCTGT | |
| | | GGCTGGGCCTGGCCCTGCTGGGCGCCCTGCACGCCCAGGCCGA | |
| | | GATGTGGCACGAGGGCCTGGAGGAGGCCAGCAGGCTGTATTTT | |
| | | GGCGAGCGCAACGTGAAGGGCATGTTCGAGGTGCTGGAGCCTC | |
| | | TGCACGCCATGATGGAGAGAGGCCCACAGACCCTGAAGGAGAC | |
| | | ATCCTTTAACCAGGCCTATGGACGGGACCTGATGGAGGCACAG | |
| | | GAGTGGTGCAGAAAGTACATGAAGTCTGGCAATGTGAAGGACC | |
| | | TGACGCAGGCCTGGGATCTGTACTATCACGTGTTTCGGAGAAT | |
| | | CTCCAAGGGCAAAGACACGATTCCGTGGCTTGGGCATCTGCTC | |
| | | GTTGGGCTGAGTGGTGCGTTTGGTTTCATCATCTTGGTCTATC | |
| | | TCTTGATCAATTGCAGAAATACAGGCCCTTGGCTGAAAAAAGT | |
| | | GCTCAAGTGTAATACCCCCGACCCAAGCAAGTTCTTCTCCCAG | |
| | | CTTTCTTCAGAGCATGGAGGCGATGTGCAGAAATGGCTCTCTT | |
| | | CACCTTTTCCCTCCTCAAGTTTCTCCCCGGGAGGGCTGGCGCC | |
| | | CGAGATTTCACCTCTTGAGGTACTTGAACGAGACAAGGTTACC | |
| | | CAACTTCTCCTTCAACAGGATAAGGTACCCGAACCTGCGAGCC | |
| | | TTAGCTCCAACCACTCTCTTACGAGCTGCTTCACCAATCAGGG | |
| | | ATACTTCTTTTTCCACCTTCCCGATGCGCTGGAAATCGAAGCA | |
| | | TGTCAAGTTTACTTTACCTATGATCCATATAGCGAGGAAGATC | |
| | | CCGACGAAGGAGTCGCCGGTGCGCCCACGGGTTCCTCACCCCA | |
| | | ACCTCTCCAGCCTCTCTCAGGAGAAGATGATGCTTATTGCACT | |
| | | TTTCCCAGTAGAGACGATCTCCTCCTCTTTTCTCCATCTCTTT | |
| | | TGGGGGGACCTTCCCCCCCTTCTACGGCACCTGGCGGGTCTGG | |
| | | TGCTGGCGAGGAGCGGATGCCGCCGTCCCTCCAGGAGCGAGTA | |
| | | CCACGAGATTGGGATCCCCAGCCACTTGGACCACCAACTCCAG | |
| | | GCGTACCTGACCTTGTCGATTTTCAACCTCCCCCTGAATTGGT | |
| | | GCTGCGAGAGGCTGGGGAGGAAGTTCCGGACGCTGGGCCGAGG | |
| | | GAGGGCGTGTCCTTTCCATGGAGTAGGCCTCCAGGTCAAGGCG | |
| | | AGTTTAGGGCTCTCAACGCGCGGCTGCCGTTGAATACAGACGC | |
| | | TTATCTCTCACTGCAGGAACTGCAAGGTCAGGACCCAACACAT | |
| | | CTTGTAGGATCCGGCGCCACCAACTTTAGTCTGCTCAAGCAAG | |
| | | CCGGGGACGTCGAGGAAAATCCTGGGCCAGAAATGTGGCACGA | |
| | | AGGACTCGAGGAAGCCAGTCGGCTGTATTTTGGCGAGCGGAAT | |
| | | GTGAAAGGGATGTTTGAAGTGCTCGAGCCTCTCCACGCTATGA | |
| | | TGGAACGGGGACCCCAGACTCTCAAAGAAACCAGCTTTAATCA | |
| | | GGCTTACGGACGCGACCTCATGGAAGCTCAAGAATGGTGTAGA | |
| | | AAGTATATGAAGAGTGGCAACGTGAAAGATCTGACACAAGCCT | |
| | | GGGATCTCTATTATCACGTGTTCAGACGCATCAGCAAAGGCAG | |
| | | CGGCGCCACAAATTTCTCCCTGCTGAAACAGGCCGGCGACGTG | |
| | | GAAGAGAATCCCGGACCTATGCCCAATCCTAGACCTGGCAAGC | |
| | | CCAGCGCTCCTTCTCTTGCTCTTGGACCTTCTCCTGGTGCAAG | |
| | | TCCATCTTGGCGAGCAGCTCCAAAGGCT | |
| | Full-length | ACTGCCACCAGCCCATTCTGTGCCAGCATCATCACTTGCCAGG | 173 |
| | donor | ACTGTTACAATAGCCTCCTCACTAGCCCCACTCACAGCAGCCA | |
| | template | GATGAATCTTTTGAGTCCATGCCTAGTCACTGGGGCAAAATAG | |
| | | GACTCCGAGGAGAAAGTCCGAGACCAGCTCCGGCAAGATGAGC | |
| | | AAACACAGCCTGTGCAGGGTGCAGGGAGGGCTAGAGGCCTGAG | |
| | | GCTTGAAACAGCTCTCAAGTGGAGGGGGAAACAACCATTGCCC | |
| | | TCATAGAGGACACATCCACACCAGGGCTGTGCTAGCGTGGGCA | |
| | | GGCAAGCCAGGTGCTGGACCTCTGCACGTGGGGCATGTGTGGG | |
| | | TATGTACATGTACCTGTGTTCTTGGTGTGTGTGTGTGTGTGTG | |
| | | TGTGTGTGTGTGTCTAGAGCTGGGGTGCAACTATGGGGCCCCT | |
| | | CGGGACATGTCCCAGCCAATGCCTGCTTTGACCAGAGGAGTGT | |
| | | CCACGTGGCTCAGGTGGTCGAGTATCTCATACCGCCCTAGCAC | |
| | | ACGTGTGACTCCTTTCCCCTATTGTCTACGCAGCCTGCCCTTG | |
| | | GACAAGGACCCGATGCCCAACCCCAGGCCTGGCAAGCCCTCGG | |
| | | CCCCTTCCTTGGCCCTTGGCCCATCCCCAGGAGCCTCGCCCAG | |
| | | CTGGAGAGCTGCATGAACAGAGAAACAGGAGAATATGGGCCAA | |
| | | ACAGGATATCTGTGGTAAGCAGTTCCTGCCCCGGCTCAGGGCC | |
| | | AAGAACAGTTGGAACAGCAGAATATGGGCCAAACAGGATATCT | |
| | | GTGGTAAGCAGTTCCTGCCCCGGCTCAGGGCCAAGAACAGATG | |
| | | GTCCCCAGATGCGGTCCCGCCCTCAGCAGTTTCTAGAGAACCA | |
| | | TCAGATGTTTCCAGGGTGCCCCAAGGACCTGAAATGACCCTGT | |
| | | GCCTTATTTGAACTAACCAATCAGTTCGCTTCTCGCTTCTGTT | |
| | | CGCGCGCTTCTGCTCCCCGAGCTCTATATAAGCAGAGCTCGTT | |
| | | TAGTGAACCGTCAGATCGCCTGGAGACGCCATCCACGCTGTTT | |
| | | TGACTTCCATAGAAGGATCTCGAGGCCACCATGCCTCTGGGCC | |
| | | TGCTGTGGCTGGGCCTGGCCCTGCTGGGCGCCCTGCACGCCCA | |
| | | GGCCGAGATGTGGCACGAGGGCCTGGAGGAGGCCAGCAGGCTG | |
| | | TATTTTGGCGAGCGCAACGTGAAGGGCATGTTCGAGGTGCTGG | |
| | | AGCCTCTGCACGCCATGATGGAGAGAGGCCCACAGACCCTGAA | |
| | | GGAGACATCCTTTAACCAGGCCTATGGACGGGACCTGATGGAG | |
| | | GCACAGGAGTGGTGCAGAAAGTACATGAAGTCTGGCAATGTGA | |
| | | AGGACCTGACGCAGGCCTGGGATCTGTACTATCACGTGTTTCG | |
| | | GAGAATCTCCAAGGGCAAAGACACGATTCCGTGGCTTGGGCAT | |
| | | CTGCTCGTTGGGCTGAGTGGTGCGTTTGGTTTCATCATCTTGG | |
| | | TCTATCTCTTGATCAATTGCAGAAATACAGGCCCTTGGCTGAA | |
| | | AAAAGTGCTCAAGTGTAATACCCCCGACCCAAGCAAGTTCTTC | |
| | | TCCCAGCTTTCTTCAGAGCATGGAGGCGATGTGCAGAAATGGC | |
| | | TCTCTTCACCTTTTCCCTCCTCAAGTTTCTCCCCGGGAGGGCT | |
| | | GGCGCCCGAGATTTCACCTCTTGAGGTACTTGAACGAGACAAG | |
| | | GTTACCCAACTTCTCCTTCAACAGGATAAGGTACCCGAACCTG | |
| | | CGAGCCTTAGCTCCAACCACTCTCTTACGAGCTGCTTCACCAA | |
| | | TCAGGGATACTTCTTTTTCCACCTTCCCGATGCGCTGGAAATC | |
| | | GAAGCATGTCAAGTTTACTTTACCTATGATCCATATAGCGAGG | |
| | | AAGATCCCGACGAAGGAGTCGCCGGTGCGCCCACGGGTTCCTC | |
| | | ACCCCAACCTCTCCAGCCTCTCTCAGGAGAAGATGATGCTTAT | |
| | | TGCACTTTTCCCAGTAGAGACGATCTCCTCCTCTTTTCTCCAT | |
| | | CTCTTTTGGGGGGACCTTCCCCCCCTTCTACGGCACCTGGCGG | |
| | | GTCTGGTGCTGGCGAGGAGCGGATGCCGCCGTCCCTCCAGGAG | |
| | | CGAGTACCACGAGATTGGGATCCCCAGCCACTTGGACCACCAA | |
| | | CTCCAGGCGTACCTGACCTTGTCGATTTTCAACCTCCCCCTGA | |
| | | ATTGGTGCTGCGAGAGGCTGGGGAGGAAGTTCCGGACGCTGGG | |
| | | CCGAGGGAGGGCGTGTCCTTTCCATGGAGTAGGCCTCCAGGTC | |
| | | AAGGCGAGTTTAGGGCTCTCAACGCGCGGCTGCCGTTGAATAC | |
| | | AGACGCTTATCTCTCACTGCAGGAACTGCAAGGTCAGGACCCA | |
| | | ACACATCTTGTAGGATCCGGCGCCACCAACTTTAGTCTGCTCA | |
| | | AGCAAGCCGGGGACGTCGAGGAAAATCCTGGGCCAGAAATGTG | |
| | | GCACGAAGGACTCGAGGAAGCCAGTCGGCTGTATTTTGGCGAG | |
| | | CGGAATGTGAAAGGGATGTTTGAAGTGCTCGAGCCTCTCCACG | |
| | | CTATGATGGAACGGGGACCCCAGACTCTCAAAGAAACCAGCTT | |
| | | TAATCAGGCTTACGGACGCGACCTCATGGAAGCTCAAGAATGG | |
| | | TGTAGAAAGTATATGAAGAGTGGCAACGTGAAAGATCTGACAC | |
| | | AAGCCTGGGATCTCTATTATCACGTGTTCAGACGCATCAGCAA | |
| | | AGGCAGCGGCGCCACAAATTTCTCCCTGCTGAAACAGGCCGGC | |
| | | GACGTGGAAGAGAATCCCGGACCTATGCCCAATCCTAGACCTG | |
| | | GCAAGCCCAGCGCTCCTTCTCTTGCTCTTGGACCTTCTCCTGG | |
| | | TGCAAGTCCATCTTGGCGAGCAGCTCCAAAGGCTTCAGACCTG | |
| | | CTGGGGGCCCGGGGCCCAGGGGGAACCTTCCAGGGCCGAGATC | |
| | | TTCGAGGCGGGGCCCATGCCTCCTCTTCTTCCTTGAACCCCAT | |
| | | GCCACCATCGCAGCTGCAGGTGAGGCCCTGGGCCCAGGATGGG | |
| | | GCAGGCAGGGTGGGGTACCTGGACCTACAGGTGCCGACCTTTA | |
| | | CTGTGGCACTGGGCGGGAGGGGGGCTGGCTGGGGCACAGGAAG | |
| | | TGGTTTCTGGGTCCCAGGCAAGTCTGTGACTTATGCAGATGTT | |
| | | GCAGGGCCAAGAAAATCCCCACCTGCCAGGCCTCAGAGATTGG | |
| | | AGGCTCTCCCCGACCTCCCAATCCCTGTCTCAGGAGAGGAGGA | |
| | | GGCCGTATTGTAGTCCCATGAGCATAGCTATGTGTCCCCATCC | |
| | | CCATGTGACAAGAGAAGAGGACTGGGGCCAAGTAGGTGAGGTG | |
| | | ACAGGGCTGAGGCCAGCTCTGCAACTTATTAGCTGTTTGATCT | |
| | | TTAAAAAGTTACTCGATCTCCATGAGCCTCAGTTTCCATACGT | |
| | | GTAAAAGGGGGATGATCATAGCATCTACCATGTGGGCTTGCAG | |
| | | TGCAGAGTATTTGAATTAGACACAGAACAGTGAGGATCAGGAT | |
| | | GGCCTCTCACCCACCTGCCTTTCTGCCCAGCTGCCCACA | |
| FoxP3_v4 | 5′ Homology | ACTTGCCAGGACTGTTACAATAGCCTCCTCACTAGCCCCACTC | 174 |
| | Arm | ACAGCAGCCAGATGAATCTTTTGAGTCCATGCCTAGTCACTGG | |
| | | GGCAAAATAGGACTCCGAGGAGAAAGTCCGAGACCAGCTCCGG | |
| | | CAAGATGAGCAAACACAGCCTGTGCAGGGTGCAGGGAGGGCTA | |
| | | GAGGCCTGAGGCTTGAAACAGCTCTCAAGTGGAGGGGGAAACA | |
| | | ACCATTGCCCTCATAGAGGACACATCCACACCAGGGCTGTGCT | |
| | | AGCGTGGGCAGGCAAGCCAGGTGCTGGACCTCTGCACGTGGGG | |
| | | CATGTGTGGGTATGTACATGTACCTGTGTTCTTGGTGTGTGTG | |
| | | TGTGTGTGTGTGTGTGTGTGTGTCTAGAGCTGGGGTGCAACTA | |
| | | TGGGGCCCCTCGGGACATGTCCCAGCCAATGCCTGCTTTGACC | |
| | | AGAGGAGTGTCCACGTGGCTCAGGTGGTCGAGTATCTCATACC | |
| | | GCCCTAGCACACGTGTGACTCCTTTCCCCTATTGTCTACGCAG | |
| | | CCTGCCCTTGGACAAGGACCCGATGCCCAACCCCAGGCCTGGC | |
| | | AAGCCCTCGGCCCCTTCCTTGGCCCTTGGCCCATCCCCAGGAG | |
| | | CCTCGCCCAGCTGGAGGGCTGCACCCAAAGCCTCAGACCTGCT | |
| | | GGGGGCCCGGGG | |
| | Intervening | ACCAGGTGGAACATTCT | 175 |
| | sequence | | |
| | MND Promoter | GAACAGAGAAACAGGAGAATATGGGCCAAACAGGATATCTGTG | 176 |
| | | GTAAGCAGTTCCTGCCCCGGCTCAGGGCCAAGAACAGTTGGAA | |
| | | CAGCAGAATATGGGCCAAACAGGATATCTGTGGTAAGCAGTTC | |
| | | CTGCCCCGGCTCAGGGCCAAGAACAGATGGTCCCCAGATGCGG | |
| | | TCCCGCCCTCAGCAGTTTCTAGAGAACCATCAGATGTTTCCAG | |
| | | GGTGCCCCAAGGACCTGAAATGACCCTGTGCCTTATTTGAACT | |
| | | AACCAATCAGTTCGCTTCTCGCTTCTGTTCGCGCGCTTCTGCT | |
| | | CCCCGAGCTCTATATAAGCAGAGCTCGTTTAGTGAACCGTCAG | |
| | | ATC | |
| | Intervening | GCCTGGAGACGCCATCCACGCTGTTTTGACTTCCATAGAAGGA | 177 |
| | Sequence | TCTCGAGGCCACCATGCCTCTGGGCCTGCTGTGGCTGGGCCTG | |
| | (LCN2 signal | GCCCTGCTGGGCGCCCTGCACGCCCAGGCC | |
| | peptide of | | |
| | FRB-IL2Rb | | |
| | underlined) | | |
| | FRB-IL2Rb | GAGATGTGGCACGAGGGCCTGGAGGAGGCCAGCAGGCTGTATT | 178 |
| | | TTGGCGAGCGCAACGTGAAGGGCATGTTCGAGGTGCTGGAGCC | |
| | | TCTGCACGCCATGATGGAGAGAGGCCCACAGACCCTGAAGGAG | |
| | | ACATCCTTTAACCAGGCCTATGGACGGGACCTGATGGAGGCAC | |
| | | AGGAGTGGTGCAGAAAGTACATGAAGTCTGGCAATGTGAAGGA | |
| | | CCTGACGCAGGCCTGGGATCTGTACTATCACGTGTTTCGGAGA | |
| | | ATCTCCAAGGGCAAAGACACGATTCCGTGGCTTGGGCATCTGC | |
| | | TCGTTGGGCTGAGTGGTGCGTTTGGTTTCATCATCTTGGTCTA | |
| | | TCTCTTGATCAATTGCAGAAATACAGGCCCTTGGCTGAAAAAA | |
| | | GTGCTCAAGTGTAATACCCCCGACCCAAGCAAGTTCTTCTCCC | |
| | | AGCTTTCTTCAGAGCATGGAGGCGATGTGCAGAAATGGCTCTC | |
| | | TTCACCTTTTCCCTCCTCAAGTTTCTCCCCGGGAGGGCTGGCG | |
| | | CCCGAGATTTCACCTCTTGAGGTACTTGAACGAGACAAGGTTA | |
| | | CCCAACTTCTCCTTCAACAGGATAAGGTACCCGAACCTGCGAG | |
| | | CCTTAGCTCCAACCACTCTCTTACGAGCTGCTTCACCAATCAG | |
| | | GGATACTTCTTTTTCCACCTTCCCGATGCGCTGGAAATCGAAG | |
| | | CATGTCAAGTTTACTTTACCTATGATCCATATAGCGAGGAAGA | |
| | | TCCCGACGAAGGAGTCGCCGGTGCGCCCACGGGTTCCTCACCC | |
| | | CAACCTCTCCAGCCTCTCTCAGGAGAAGATGATGCTTATTGCA | |
| | | CTTTTCCCAGTAGAGACGATCTCCTCCTCTTTTCTCCATCTCT | |
| | | TTTGGGGGGACCTTCCCCCCCTTCTACGGCACCTGGCGGGTCT | |
| | | GGTGCTGGCGAGGAGCGGATGCCGCCGTCCCTCCAGGAGCGAG | |
| | | TACCACGAGATTGGGATCCCCAGCCACTTGGACCACCAACTCC | |
| | | AGGCGTACCTGACCTTGTCGATTTTCAACCTCCCCCTGAATTG | |
| | | GTGCTGCGAGAGGCTGGGGAGGAAGTTCCGGACGCTGGGCCGA | |
| | | GGGAGGGCGTGTCCTTTCCATGGAGTAGGCCTCCAGGTCAAGG | |
| | | CGAGTTTAGGGCTCTCAACGCGCGGCTGCCGTTGAATACAGAC | |
| | | GCTTATCTCTCACTGCAGGAACTGCAAGGTCAGGACCCAACAC | |
| | | ATCTTGTA | |
| | GSG linker | GGATCCGGC | |
| | P2A | GCCACCAACTTTAGTCTGCTCAAGCAAGCCGGGGACGTCGAGG | 179 |
| | | AAAATCCTGGGCCA | |
| | Cytosolic | GAAATGTGGCACGAAGGACTCGAGGAAGCCAGTCGGCTGTATT | 180 |
| | FRB | TTGGCGAGCGGAATGTGAAAGGGATGTTTGAAGTGCTCGAGCC | |
| | | TCTCCACGCTATGATGGAACGGGGACCCCAGACTCTCAAAGAA | |
| | | ACCAGCTTTAATCAGGCTTACGGACGCGACCTCATGGAAGCTC | |
| | | AAGAATGGTGTAGAAAGTATATGAAGAGTGGCAACGTGAAAGA | |
| | | TCTGACACAAGCCTGGGATCTCTATTATCACGTGTTCAGACGC | |
| | | ATCAGCAAA | |
| | GSG linker | GGCAGCGGC | |
| | Second P2A | GCCACAAATTTCTCCCTGCTGAAACAGGCCGGCGACGTGGAAG | 181 |
| | | AGAATCCCGGACCT | |
| | FoxP3 | ATGCCCAATCCTAGACCTGGCAAGCCCAGCGCTCCTTCTCTTG | 182 |
| | portion | CTCTTGGACCTTCTCCTGGTGCAAGTCCATCTTGGCGAGCAGC | |
| | | TCCAAAGGCTAGTGATCTTCTCGGAGCTAGAGGTCCTGGAGGC | |
| | | ACATTTCAGGGA | |
| | 3′ Homology | CGAGATCTTCGAGGCGGGGCCCATGCCTCCTCTTCTTCCTTGA | 183 |
| | Arm | ACCCCATGCCACCATCGCAGCTGCAGGTGAGGCCCTGGGCCCA | |
| | | GGATGGGGCAGGCAGGGTGGGGTACCTGGACCTACAGGTGCCG | |
| | | ACCTTTACTGTGGCACTGGGCGGGAGGGGGGCTGGCTGGGGCA | |
| | | CAGGAAGTGGTTTCTGGGTCCCAGGCAAGTCTGTGACTTATGC | |
| | | AGATGTTGCAGGGCCAAGAAAATCCCCACCTGCCAGGCCTCAG | |
| | | AGATTGGAGGCTCTCCCCGACCTCCCAATCCCTGTCTCAGGAG | |
| | | AGGAGGAGGCCGTATTGTAGTCCCATGAGCATAGCTATGTGTC | |
| | | CCCATCCCCATGTGACAAGAGAAGAGGACTGGGGCCAAGTAGG | |
| | | TGAGGTGACAGGGCTGAGGCCAGCTCTGCAACTTATTAGCTGT | |
| | | TTGATCTTTAAAAAGTTACTCGATCTCCATGAGCCTCAGTTTC | |
| | | CATACGTGTAAAAGGGGGATGATCATAGCATCTACCATGTGGG | |
| | | CTTGCAGTGCAGAGTATTTGAATTAGACACAGAACAGTGAGGA | |
| | | TCAGGATGGCCTCTCACCCACCTGCCTTTCTGCCCAGCTGCCC | |
| | | ACACTGCCCCTAGTCATGGTGGCACCCTCCGGGGCACGGCTGG | |
| | | GCCCCT | |
| | Between | ACCAGGTGGAACATTCTGAACAGAGAAACAGGAGAATATGGGC | 184 |
| | Homology | CAAACAGGATATCTGTGGTAAGCAGTTCCTGCCCCGGCTCAGG | |
| | Arms | GCCAAGAACAGTTGGAACAGCAGAATATGGGCCAAACAGGATA | |
| | | TCTGTGGTAAGCAGTTCCTGCCCCGGCTCAGGGCCAAGAACAG | |
| | | ATGGTCCCCAGATGCGGTCCCGCCCTCAGCAGTTTCTAGAGAA | |
| | | CCATCAGATGTTTCCAGGGTGCCCCAAGGACCTGAAATGACCC | |
| | | TGTGCCTTATTTGAACTAACCAATCAGTTCGCTTCTCGCTTCT | |
| | | GTTCGCGCGCTTCTGCTCCCCGAGCTCTATATAAGCAGAGCTC | |
| | | GTTTAGTGAACCGTCAGATCGCCTGGAGACGCCATCCACGCTG | |
| | | TTTTGACTTCCATAGAAGGATCTCGAGGCCACCATGCCTCTGG | |
| | | GCCTGCTGTGGCTGGGCCTGGCCCTGCTGGGCGCCCTGCACGC | |
| | | CCAGGCCGAGATGTGGCACGAGGGCCTGGAGGAGGCCAGCAGG | |
| | | CTGTATTTTGGCGAGCGCAACGTGAAGGGCATGTTCGAGGTGC | |
| | | TGGAGCCTCTGCACGCCATGATGGAGAGAGGCCCACAGACCCT | |
| | | GAAGGAGACATCCTTTAACCAGGCCTATGGACGGGACCTGATG | |
| | | GAGGCACAGGAGTGGTGCAGAAAGTACATGAAGTCTGGCAATG | |
| | | TGAAGGACCTGACGCAGGCCTGGGATCTGTACTATCACGTGTT | |
| | | TCGGAGAATCTCCAAGGGCAAAGACACGATTCCGTGGCTTGGG | |
| | | CATCTGCTCGTTGGGCTGAGTGGTGCGTTTGGTTTCATCATCT | |
| | | TGGTCTATCTCTTGATCAATTGCAGAAATACAGGCCCTTGGCT | |
| | | GAAAAAAGTGCTCAAGTGTAATACCCCCGACCCAAGCAAGTTC | |
| | | TTCTCCCAGCTTTCTTCAGAGCATGGAGGCGATGTGCAGAAAT | |
| | | GGCTCTCTTCACCTTTTCCCTCCTCAAGTTTCTCCCCGGGAGG | |
| | | GCTGGCGCCCGAGATTTCACCTCTTGAGGTACTTGAACGAGAC | |
| | | AAGGTTACCCAACTTCTCCTTCAACAGGATAAGGTACCCGAAC | |
| | | CTGCGAGCCTTAGCTCCAACCACTCTCTTACGAGCTGCTTCAC | |
| | | CAATCAGGGATACTTCTTTTTCCACCTTCCCGATGCGCTGGAA | |
| | | ATCGAAGCATGTCAAGTTTACTTTACCTATGATCCATATAGCG | |
| | | AGGAAGATCCCGACGAAGGAGTCGCCGGTGCGCCCACGGGTTC | |
| | | CTCACCCCAACCTCTCCAGCCTCTCTCAGGAGAAGATGATGCT | |
| | | TATTGCACTTTTCCCAGTAGAGACGATCTCCTCCTCTTTTCTC | |
| | | CATCTCTTTTGGGGGGACCTTCCCCCCCTTCTACGGCACCTGG | |
| | | CGGGTCTGGTGCTGGCGAGGAGCGGATGCCGCCGTCCCTCCAG | |
| | | GAGCGAGTACCACGAGATTGGGATCCCCAGCCACTTGGACCAC | |
| | | CAACTCCAGGCGTACCTGACCTTGTCGATTTTCAACCTCCCCC | |
| | | TGAATTGGTGCTGCGAGAGGCTGGGGAGGAAGTTCCGGACGCT | |
| | | GGGCCGAGGGAGGGCGTGTCCTTTCCATGGAGTAGGCCTCCAG | |
| | | GTCAAGGCGAGTTTAGGGCTCTCAACGCGCGGCTGCCGTTGAA | |
| | | TACAGACGCTTATCTCTCACTGCAGGAACTGCAAGGTCAGGAC | |
| | | CCAACACATCTTGTAGGATCCGGCGCCACCAACTTTAGTCTGC | |
| | | TCAAGCAAGCCGGGGACGTCGAGGAAAATCCTGGGCCAGAAAT | |
| | | GTGGCACGAAGGACTCGAGGAAGCCAGTCGGCTGTATTTTGGC | |
| | | GAGCGGAATGTGAAAGGGATGTTTGAAGTGCTCGAGCCTCTCC | |
| | | ACGCTATGATGGAACGGGGACCCCAGACTCTCAAAGAAACCAG | |
| | | CTTTAATCAGGCTTACGGACGCGACCTCATGGAAGCTCAAGAA | |
| | | TGGTGTAGAAAGTATATGAAGAGTGGCAACGTGAAAGATCTGA | |
| | | CACAAGCCTGGGATCTCTATTATCACGTGTTCAGACGCATCAG | |
| | | CAAAGGCAGCGGCGCCACAAATTTCTCCCTGCTGAAACAGGCC | |
| | | GGCGACGTGGAAGAGAATCCCGGACCTATGCCCAATCCTAGAC | |
| | | CTGGCAAGCCCAGCGCTCCTTCTCTTGCTCTTGGACCTTCTCC | |
| | | TGGTGCAAGTCCATCTTGGCGAGCAGCTCCAAAGGCTAGTGAT | |
| | | CTTCTCGGAGCTAGAGGTCCTGGAGGCACATTTCAGGGA | |
| | Full-length | ACTTGCCAGGACTGTTACAATAGCCTCCTCACTAGCCCCACTC | 185 |
| | donor | ACAGCAGCCAGATGAATCTTTTGAGTCCATGCCTAGTCACTGG | |
| | template | GGCAAAATAGGACTCCGAGGAGAAAGTCCGAGACCAGCTCCGG | |
| | | CAAGATGAGCAAACACAGCCTGTGCAGGGTGCAGGGAGGGCTA | |
| | | GAGGCCTGAGGCTTGAAACAGCTCTCAAGTGGAGGGGGAAACA | |
| | | ACCATTGCCCTCATAGAGGACACATCCACACCAGGGCTGTGCT | |
| | | AGCGTGGGCAGGCAAGCCAGGTGCTGGACCTCTGCACGTGGGG | |
| | | CATGTGTGGGTATGTACATGTACCTGTGTTCTTGGTGTGTGTG | |
| | | TGTGTGTGTGTGTGTGTGTGTGTCTAGAGCTGGGGTGCAACTA | |
| | | TGGGGCCCCTCGGGACATGTCCCAGCCAATGCCTGCTTTGACC | |
| | | AGAGGAGTGTCCACGTGGCTCAGGTGGTCGAGTATCTCATACC | |
| | | GCCCTAGCACACGTGTGACTCCTTTCCCCTATTGTCTACGCAG | |
| | | CCTGCCCTTGGACAAGGACCCGATGCCCAACCCCAGGCCTGGC | |
| | | AAGCCCTCGGCCCCTTCCTTGGCCCTTGGCCCATCCCCAGGAG | |
| | | CCTCGCCCAGCTGGAGGGCTGCACCCAAAGCCTCAGACCTGCT | |
| | | GGGGGCCCGGGGACCAGGTGGAACATTCTGAACAGAGAAACAG | |
| | | GAGAATATGGGCCAAACAGGATATCTGTGGTAAGCAGTTCCTG | |
| | | CCCCGGCTCAGGGCCAAGAACAGTTGGAACAGCAGAATATGGG | |
| | | CCAAACAGGATATCTGTGGTAAGCAGTTCCTGCCCCGGCTCAG | |
| | | GGCCAAGAACAGATGGTCCCCAGATGCGGTCCCGCCCTCAGCA | |
| | | GTTTCTAGAGAACCATCAGATGTTTCCAGGGTGCCCCAAGGAC | |
| | | CTGAAATGACCCTGTGCCTTATTTGAACTAACCAATCAGTTCG | |
| | | CTTCTCGCTTCTGTTCGCGCGCTTCTGCTCCCCGAGCTCTATA | |
| | | TAAGCAGAGCTCGTTTAGTGAACCGTCAGATCGCCTGGAGACG | |
| | | CCATCCACGCTGTTTTGACTTCCATAGAAGGATCTCGAGGCCA | |
| | | CCATGCCTCTGGGCCTGCTGTGGCTGGGCCTGGCCCTGCTGGG | |
| | | CGCCCTGCACGCCCAGGCCGAGATGTGGCACGAGGGCCTGGAG | |
| | | GAGGCCAGCAGGCTGTATTTTGGCGAGCGCAACGTGAAGGGCA | |
| | | TGTTCGAGGTGCTGGAGCCTCTGCACGCCATGATGGAGAGAGG | |
| | | CCCACAGACCCTGAAGGAGACATCCTTTAACCAGGCCTATGGA | |
| | | CGGGACCTGATGGAGGCACAGGAGTGGTGCAGAAAGTACATGA | |
| | | AGTCTGGCAATGTGAAGGACCTGACGCAGGCCTGGGATCTGTA | |
| | | CTATCACGTGTTTCGGAGAATCTCCAAGGGCAAAGACACGATT | |
| | | CCGTGGCTTGGGCATCTGCTCGTTGGGCTGAGTGGTGCGTTTG | |
| | | GTTTCATCATCTTGGTCTATCTCTTGATCAATTGCAGAAATAC | |
| | | AGGCCCTTGGCTGAAAAAAGTGCTCAAGTGTAATACCCCCGAC | |
| | | CCAAGCAAGTTCTTCTCCCAGCTTTCTTCAGAGCATGGAGGCG | |
| | | ATGTGCAGAAATGGCTCTCTTCACCTTTTCCCTCCTCAAGTTT | |
| | | CTCCCCGGGAGGGCTGGCGCCCGAGATTTCACCTCTTGAGGTA | |
| | | CTTGAACGAGACAAGGTTACCCAACTTCTCCTTCAACAGGATA | |
| | | AGGTACCCGAACCTGCGAGCCTTAGCTCCAACCACTCTCTTAC | |
| | | GAGCTGCTTCACCAATCAGGGATACTTCTTTTTCCACCTTCCC | |
| | | GATGCGCTGGAAATCGAAGCATGTCAAGTTTACTTTACCTATG | |
| | | ATCCATATAGCGAGGAAGATCCCGACGAAGGAGTCGCCGGTGC | |
| | | GCCCACGGGTTCCTCACCCCAACCTCTCCAGCCTCTCTCAGGA | |
| | | GAAGATGATGCTTATTGCACTTTTCCCAGTAGAGACGATCTCC | |
| | | TCCTCTTTTCTCCATCTCTTTTGGGGGGACCTTCCCCCCCTTC | |
| | | TACGGCACCTGGCGGGTCTGGTGCTGGCGAGGAGCGGATGCCG | |
| | | CCGTCCCTCCAGGAGCGAGTACCACGAGATTGGGATCCCCAGC | |
| | | CACTTGGACCACCAACTCCAGGCGTACCTGACCTTGTCGATTT | |
| | | TCAACCTCCCCCTGAATTGGTGCTGCGAGAGGCTGGGGAGGAA | |
| | | GTTCCGGACGCTGGGCCGAGGGAGGGCGTGTCCTTTCCATGGA | |
| | | GTAGGCCTCCAGGTCAAGGCGAGTTTAGGGCTCTCAACGCGCG | |
| | | GCTGCCGTTGAATACAGACGCTTATCTCTCACTGCAGGAACTG | |
| | | CAAGGTCAGGACCCAACACATCTTGTAGGATCCGGCGCCACCA | |
| | | ACTTTAGTCTGCTCAAGCAAGCCGGGGACGTCGAGGAAAATCC | |
| | | TGGGCCAGAAATGTGGCACGAAGGACTCGAGGAAGCCAGTCGG | |
| | | CTGTATTTTGGCGAGCGGAATGTGAAAGGGATGTTTGAAGTGC | |
| | | TCGAGCCTCTCCACGCTATGATGGAACGGGGACCCCAGACTCT | |
| | | CAAAGAAACCAGCTTTAATCAGGCTTACGGACGCGACCTCATG | |
| | | GAAGCTCAAGAATGGTGTAGAAAGTATATGAAGAGTGGCAACG | |
| | | TGAAAGATCTGACACAAGCCTGGGATCTCTATTATCACGTGTT | |
| | | CAGACGCATCAGCAAAGGCAGCGGCGCCACAAATTTCTCCCTG | |
| | | CTGAAACAGGCCGGCGACGTGGAAGAGAATCCCGGACCTATGC | |
| | | CCAATCCTAGACCTGGCAAGCCCAGCGCTCCTTCTCTTGCTCT | |
| | | TGGACCTTCTCCTGGTGCAAGTCCATCTTGGCGAGCAGCTCCA | |
| | | AAGGCTAGTGATCTTCTCGGAGCTAGAGGTCCTGGAGGCACAT | |
| | | TTCAGGGACGAGATCTTCGAGGCGGGGCCCATGCCTCCTCTTC | |
| | | TTCCTTGAACCCCATGCCACCATCGCAGCTGCAGGTGAGGCCC | |
| | | TGGGCCCAGGATGGGGCAGGCAGGGTGGGGTACCTGGACCTAC | |
| | | AGGTGCCGACCTTTACTGTGGCACTGGGCGGGAGGGGGGCTGG | |
| | | CTGGGGCACAGGAAGTGGTTTCTGGGTCCCAGGCAAGTCTGTG | |
| | | ACTTATGCAGATGTTGCAGGGCCAAGAAAATCCCCACCTGCCA | |
| | | GGCCTCAGAGATTGGAGGCTCTCCCCGACCTCCCAATCCCTGT | |
| | | CTCAGGAGAGGAGGAGGCCGTATTGTAGTCCCATGAGCATAGC | |
| | | TATGTGTCCCCATCCCCATGTGACAAGAGAAGAGGACTGGGGC | |
| | | CAAGTAGGTGAGGTGACAGGGCTGAGGCCAGCTCTGCAACTTA | |
| | | TTAGCTGTTTGATCTTTAAAAAGTTACTCGATCTCCATGAGCC | |
| | | TCAGTTTCCATACGTGTAAAAGGGGGATGATCATAGCATCTAC | |
| | | CATGTGGGCTTGCAGTGCAGAGTATTTGAATTAGACACAGAAC | |
| | | AGTGAGGATCAGGATGGCCTCTCACCCACCTGCCTTTCTGCCC | |
| | | AGCTGCCCACACTGCCCCTAGTCATGGTGGCACCCTCCGGGGC | |
| | | ACGGCTGGGCCCCT | |
| FoxP3_v5 | 5′ Homology | GCCAGATGAATCTTTTGAGTCCATGCCTAGTCACTGGGGCAAA | 186 |
| | Arm | ATAGGACTCCGAGGAGAAAGTCCGAGACCAGCTCCGGCAAGAT | |
| | | GAGCAAACACAGCCTGTGCAGGGTGCAGGGAGGGCTAGAGGCC | |
| | | TGAGGCTTGAAACAGCTCTCAAGTGGAGGGGGAAACAACCATT | |
| | | GCCCTCATAGAGGACACATCCACACCAGGGCTGTGCTAGCGTG | |
| | | GGCAGGCAAGCCAGGTGCTGGACCTCTGCACGTGGGGCATGTG | |
| | | TGGGTATGTACATGTACCTGTGTTCTTGGTGTGTGTGTGTGTG | |
| | | TGTGTGTGTGTGTGTGTCTAGAGCTGGGGTGCAACTATGGGGC | |
| | | CCCTCGGGACATGTCCCAGCCAATGCCTGCTTTGACCAGAGGA | |
| | | GTGTCCACGTGGCTCAGGTGGTCGAGTATCTCATACCGCCCTA | |
| | | GCACACGTGTGACTCCTTTCCCCTATTGTCTACGCAGCCTGCC | |
| | | CTTGGACAAGGACCCGATGCCCAACCCCAGGCCTGGCAAGCCC | |
| | | TCGGCCCCTTCCTTGGCCCTTGGCCCATCCCCAGGAGCCTCGC | |
| | | CCAGCTGGAGGGCTGCACCCAAAGCCTCAGACCTGCTGGGGGC | |
| | | CCGGGGCCCAGGGGGAACCTTCCAGGGCCGAGATCTTCGAGGC | |
| | | GGGGC | |
| | Intervening | TCATGCAT | |
| | sequence | | |
| | MND Promoter | GAACAGAGAAACAGGAGAATATGGGCCAAACAGGATATCTGTG | 187 |
| | | GTAAGCAGTTCCTGCCCCGGCTCAGGGCCAAGAACAGTTGGAA | |
| | | CAGCAGAATATGGGCCAAACAGGATATCTGTGGTAAGCAGTTC | |
| | | CTGCCCCGGCTCAGGGCCAAGAACAGATGGTCCCCAGATGCGG | |
| | | TCCCGCCCTCAGCAGTTTCTAGAGAACCATCAGATGTTTCCAG | |
| | | GGTGCCCCAAGGACCTGAAATGACCCTGTGCCTTATTTGAACT | |
| | | AACCAATCAGTTCGCTTCTCGCTTCTGTTCGCGCGCTTCTGCT | |
| | | CCCCGAGCTCTATATAAGCAGAGCTCGTTTAGTGAACCGTCAG | |
| | | ATC | |
| | Intervening | GCCTGGAGACGCCATCCACGCTGTTTTGACTTCCATAGAAGGA | 188 |
| | Sequence | TCTCGAGGCCACCATGCCTCTGGGCCTGCTGTGGCTGGGCCTG | |
| | (LCN2 signal | GCCCTGCTGGGCGCCCTGCACGCCCAGGCC | |
| | peptide of | | |
| | FRB-IL2Rb | | |
| | underlined) | | |
| | FRB-IL2Rb | GAGATGTGGCACGAGGGCCTGGAGGAGGCCAGCAGGCTGTATT | 189 |
| | | TTGGCGAGCGCAACGTGAAGGGCATGTTCGAGGTGCTGGAGCC | |
| | | TCTGCACGCCATGATGGAGAGAGGCCCACAGACCCTGAAGGAG | |
| | | ACATCCTTTAACCAGGCCTATGGACGGGACCTGATGGAGGCAC | |
| | | AGGAGTGGTGCAGAAAGTACATGAAGTCTGGCAATGTGAAGGA | |
| | | CCTGACGCAGGCCTGGGATCTGTACTATCACGTGTTTCGGAGA | |
| | | ATCTCCAAGGGCAAAGACACGATTCCGTGGCTTGGGCATCTGC | |
| | | TCGTTGGGCTGAGTGGTGCGTTTGGTTTCATCATCTTGGTCTA | |
| | | TCTCTTGATCAATTGCAGAAATACAGGCCCTTGGCTGAAAAAA | |
| | | GTGCTCAAGTGTAATACCCCCGACCCAAGCAAGTTCTTCTCCC | |
| | | AGCTTTCTTCAGAGCATGGAGGCGATGTGCAGAAATGGCTCTC | |
| | | TTCACCTTTTCCCTCCTCAAGTTTCTCCCCGGGAGGGCTGGCG | |
| | | CCCGAGATTTCACCTCTTGAGGTACTTGAACGAGACAAGGTTA | |
| | | CCCAACTTCTCCTTCAACAGGATAAGGTACCCGAACCTGCGAG | |
| | | CCTTAGCTCCAACCACTCTCTTACGAGCTGCTTCACCAATCAG | |
| | | GGATACTTCTTTTTCCACCTTCCCGATGCGCTGGAAATCGAAG | |
| | | CATGTCAAGTTTACTTTACCTATGATCCATATAGCGAGGAAGA | |
| | | TCCCGACGAAGGAGTCGCCGGTGCGCCCACGGGTTCCTCACCC | |
| | | CAACCTCTCCAGCCTCTCTCAGGAGAAGATGATGCTTATTGCA | |
| | | CTTTTCCCAGTAGAGACGATCTCCTCCTCTTTTCTCCATCTCT | |
| | | TTTGGGGGGACCTTCCCCCCCTTCTACGGCACCTGGCGGGTCT | |
| | | GGTGCTGGCGAGGAGCGGATGCCGCCGTCCCTCCAGGAGCGAG | |
| | | TACCACGAGATTGGGATCCCCAGCCACTTGGACCACCAACTCC | |
| | | AGGCGTACCTGACCTTGTCGATTTTCAACCTCCCCCTGAATTG | |
| | | GTGCTGCGAGAGGCTGGGGAGGAAGTTCCGGACGCTGGGCCGA | |
| | | GGGAGGGCGTGTCCTTTCCATGGAGTAGGCCTCCAGGTCAAGG | |
| | | CGAGTTTAGGGCTCTCAACGCGCGGCTGCCGTTGAATACAGAC | |
| | | GCTTATCTCTCACTGCAGGAACTGCAAGGTCAGGACCCAACAC | |
| | | ATCTTGTA | |
| | GSG linker | GGATCCGGC | |
| | P2A | GCCACCAACTTTAGTCTGCTCAAGCAAGCCGGGGACGTCGAGG | 190 |
| | | AAAATCCTGGGCCA | |
| | Cytosolic | GAAATGTGGCACGAAGGACTCGAGGAAGCCAGTCGGCTGTATT | 191 |
| | FRB | TTGGCGAGCGGAATGTGAAAGGGATGTTTGAAGTGCTCGAGCC | |
| | | TCTCCACGCTATGATGGAACGGGGACCCCAGACTCTCAAAGAA | |
| | | ACCAGCTTTAATCAGGCTTACGGACGCGACCTCATGGAAGCTC | |
| | | AAGAATGGTGTAGAAAGTATATGAAGAGTGGCAACGTGAAAGA | |
| | | TCTGACACAAGCCTGGGATCTCTATTATCACGTGTTCAGACGC | |
| | | ATCAGCAAA | |
| | GSG linker | GGCAGCGGC | |
| | Second P2A | GCCACAAATTTCTCCCTGCTGAAACAGGCCGGCGACGTGGAAG | 192 |
| | | AGAATCCCGGACCT | |
| | FoxP3 | ATGCCCAATCCTAGACCTGGCAAGCCCAGCGCTCCTTCTCTTG | 193 |
| | portion | CTCTTGGACCTTCTCCTGGTGCAAGTCCATCTTGGCGAGCAGC | |
| | | TCCAAAGGCTAGTGATCTTCTCGGAGCTAGAGGTCCTGGAGGC | |
| | | ACATTTCAAGGTAGAGACTTGAGGGGAGGTGCTCACGCT | |
| | 3′ Homology | TCCTCTTCTTCCTTGAACCCCATGCCACCATCGCAGCTGCAGG | 194 |
| | Arm | TGAGGCCCTGGGCCCAGGATGGGGCAGGCAGGGTGGGGTACCT | |
| | | GGACCTACAGGTGCCGACCTTTACTGTGGCACTGGGCGGGAGG | |
| | | GGGGCTGGCTGGGGCACAGGAAGTGGTTTCTGGGTCCCAGGCA | |
| | | AGTCTGTGACTTATGCAGATGTTGCAGGGCCAAGAAAATCCCC | |
| | | ACCTGCCAGGCCTCAGAGATTGGAGGCTCTCCCCGACCTCCCA | |
| | | ATCCCTGTCTCAGGAGAGGAGGAGGCCGTATTGTAGTCCCATG | |
| | | AGCATAGCTATGTGTCCCCATCCCCATGTGACAAGAGAAGAGG | |
| | | ACTGGGGCCAAGTAGGTGAGGTGACAGGGCTGAGGCCAGCTCT | |
| | | GCAACTTATTAGCTGTTTGATCTTTAAAAAGTTACTCGATCTC | |
| | | CATGAGCCTCAGTTTCCATACGTGTAAAAGGGGGATGATCATA | |
| | | GCATCTACCATGTGGGCTTGCAGTGCAGAGTATTTGAATTAGA | |
| | | CACAGAACAGTGAGGATCAGGATGGCCTCTCACCCACCTGCCT | |
| | | TTCTGCCCAGCTGCCCACACTGCCCCTAGTCATGGTGGCACCC | |
| | | TCCGGGGCACGGCTGGGCCCCTTGCCCCACTTACAGGCACTCC | |
| | | TCCAG | |
| | Between | TCATGCATGAACAGAGAAACAGGAGAATATGGGCCAAACAGGA | 195 |
| | Homology | TATCTGTGGTAAGCAGTTCCTGCCCCGGCTCAGGGCCAAGAAC | |
| | Arms | AGTTGGAACAGCAGAATATGGGCCAAACAGGATATCTGTGGTA | |
| | | AGCAGTTCCTGCCCCGGCTCAGGGCCAAGAACAGATGGTCCCC | |
| | | AGATGCGGTCCCGCCCTCAGCAGTTTCTAGAGAACCATCAGAT | |
| | | GTTTCCAGGGTGCCCCAAGGACCTGAAATGACCCTGTGCCTTA | |
| | | TTTGAACTAACCAATCAGTTCGCTTCTCGCTTCTGTTCGCGCG | |
| | | CTTCTGCTCCCCGAGCTCTATATAAGCAGAGCTCGTTTAGTGA | |
| | | ACCGTCAGATCGCCTGGAGACGCCATCCACGCTGTTTTGACTT | |
| | | CCATAGAAGGATCTCGAGGCCACCATGCCTCTGGGCCTGCTGT | |
| | | GGCTGGGCCTGGCCCTGCTGGGCGCCCTGCACGCCCAGGCCGA | |
| | | GATGTGGCACGAGGGCCTGGAGGAGGCCAGCAGGCTGTATTTT | |
| | | GGCGAGCGCAACGTGAAGGGCATGTTCGAGGTGCTGGAGCCTC | |
| | | TGCACGCCATGATGGAGAGAGGCCCACAGACCCTGAAGGAGAC | |
| | | ATCCTTTAACCAGGCCTATGGACGGGACCTGATGGAGGCACAG | |
| | | GAGTGGTGCAGAAAGTACATGAAGTCTGGCAATGTGAAGGACC | |
| | | TGACGCAGGCCTGGGATCTGTACTATCACGTGTTTCGGAGAAT | |
| | | CTCCAAGGGCAAAGACACGATTCCGTGGCTTGGGCATCTGCTC | |
| | | GTTGGGCTGAGTGGTGCGTTTGGTTTCATCATCTTGGTCTATC | |
| | | TCTTGATCAATTGCAGAAATACAGGCCCTTGGCTGAAAAAAGT | |
| | | GCTCAAGTGTAATACCCCCGACCCAAGCAAGTTCTTCTCCCAG | |
| | | CTTTCTTCAGAGCATGGAGGCGATGTGCAGAAATGGCTCTCTT | |
| | | CACCTTTTCCCTCCTCAAGTTTCTCCCCGGGAGGGCTGGCGCC | |
| | | CGAGATTTCACCTCTTGAGGTACTTGAACGAGACAAGGTTACC | |
| | | CAACTTCTCCTTCAACAGGATAAGGTACCCGAACCTGCGAGCC | |
| | | TTAGCTCCAACCACTCTCTTACGAGCTGCTTCACCAATCAGGG | |
| | | ATACTTCTTTTTCCACCTTCCCGATGCGCTGGAAATCGAAGCA | |
| | | TGTCAAGTTTACTTTACCTATGATCCATATAGCGAGGAAGATC | |
| | | CCGACGAAGGAGTCGCCGGTGCGCCCACGGGTTCCTCACCCCA | |
| | | ACCTCTCCAGCCTCTCTCAGGAGAAGATGATGCTTATTGCACT | |
| | | TTTCCCAGTAGAGACGATCTCCTCCTCTTTTCTCCATCTCTTT | |
| | | TGGGGGGACCTTCCCCCCCTTCTACGGCACCTGGCGGGTCTGG | |
| | | TGCTGGCGAGGAGCGGATGCCGCCGTCCCTCCAGGAGCGAGTA | |
| | | CCACGAGATTGGGATCCCCAGCCACTTGGACCACCAACTCCAG | |
| | | GCGTACCTGACCTTGTCGATTTTCAACCTCCCCCTGAATTGGT | |
| | | GCTGCGAGAGGCTGGGGAGGAAGTTCCGGACGCTGGGCCGAGG | |
| | | GAGGGCGTGTCCTTTCCATGGAGTAGGCCTCCAGGTCAAGGCG | |
| | | AGTTTAGGGCTCTCAACGCGCGGCTGCCGTTGAATACAGACGC | |
| | | TTATCTCTCACTGCAGGAACTGCAAGGTCAGGACCCAACACAT | |
| | | CTTGTAGGATCCGGCGCCACCAACTTTAGTCTGCTCAAGCAAG | |
| | | CCGGGGACGTCGAGGAAAATCCTGGGCCAGAAATGTGGCACGA | |
| | | AGGACTCGAGGAAGCCAGTCGGCTGTATTTTGGCGAGCGGAAT | |
| | | GTGAAAGGGATGTTTGAAGTGCTCGAGCCTCTCCACGCTATGA | |
| | | TGGAACGGGGACCCCAGACTCTCAAAGAAACCAGCTTTAATCA | |
| | | GGCTTACGGACGCGACCTCATGGAAGCTCAAGAATGGTGTAGA | |
| | | AAGTATATGAAGAGTGGCAACGTGAAAGATCTGACACAAGCCT | |
| | | GGGATCTCTATTATCACGTGTTCAGACGCATCAGCAAAGGCAG | |
| | | CGGCGCCACAAATTTCTCCCTGCTGAAACAGGCCGGCGACGTG | |
| | | GAAGAGAATCCCGGACCTATGCCCAATCCTAGACCTGGCAAGC | |
| | | CCAGCGCTCCTTCTCTTGCTCTTGGACCTTCTCCTGGTGCAAG | |
| | | TCCATCTTGGCGAGCAGCTCCAAAGGCTAGTGATCTTCTCGGA | |
| | | GCTAGAGGTCCTGGAGGCACATTTCAAGGTAGAGACTTGAGGG | |
| | | GAGGTGCTCACGCT | |
| | Full-length | GCCAGATGAATCTTTTGAGTCCATGCCTAGTCACTGGGGCAAA | 196 |
| | donor | ATAGGACTCCGAGGAGAAAGTCCGAGACCAGCTCCGGCAAGAT | |
| | template | GAGCAAACACAGCCTGTGCAGGGTGCAGGGAGGGCTAGAGGCC | |
| | | TGAGGCTTGAAACAGCTCTCAAGTGGAGGGGGAAACAACCATT | |
| | | GCCCTCATAGAGGACACATCCACACCAGGGCTGTGCTAGCGTG | |
| | | GGCAGGCAAGCCAGGTGCTGGACCTCTGCACGTGGGGCATGTG | |
| | | TGGGTATGTACATGTACCTGTGTTCTTGGTGTGTGTGTGTGTG | |
| | | TGTGTGTGTGTGTGTGTCTAGAGCTGGGGTGCAACTATGGGGC | |
| | | CCCTCGGGACATGTCCCAGCCAATGCCTGCTTTGACCAGAGGA | |
| | | GTGTCCACGTGGCTCAGGTGGTCGAGTATCTCATACCGCCCTA | |
| | | GCACACGTGTGACTCCTTTCCCCTATTGTCTACGCAGCCTGCC | |
| | | CTTGGACAAGGACCCGATGCCCAACCCCAGGCCTGGCAAGCCC | |
| | | TCGGCCCCTTCCTTGGCCCTTGGCCCATCCCCAGGAGCCTCGC | |
| | | CCAGCTGGAGGGCTGCACCCAAAGCCTCAGACCTGCTGGGGGC | |
| | | CCGGGGCCCAGGGGGAACCTTCCAGGGCCGAGATCTTCGAGGC | |
| | | GGGGCTCATGCATGAACAGAGAAACAGGAGAATATGGGCCAAA | |
| | | CAGGATATCTGTGGTAAGCAGTTCCTGCCCCGGCTCAGGGCCA | |
| | | AGAACAGTTGGAACAGCAGAATATGGGCCAAACAGGATATCTG | |
| | | TGGTAAGCAGTTCCTGCCCCGGCTCAGGGCCAAGAACAGATGG | |
| | | TCCCCAGATGCGGTCCCGCCCTCAGCAGTTTCTAGAGAACCAT | |
| | | CAGATGTTTCCAGGGTGCCCCAAGGACCTGAAATGACCCTGTG | |
| | | CCTTATTTGAACTAACCAATCAGTTCGCTTCTCGCTTCTGTTC | |
| | | GCGCGCTTCTGCTCCCCGAGCTCTATATAAGCAGAGCTCGTTT | |
| | | AGTGAACCGTCAGATCGCCTGGAGACGCCATCCACGCTGTTTT | |
| | | GACTTCCATAGAAGGATCTCGAGGCCACCATGCCTCTGGGCCT | |
| | | GCTGTGGCTGGGCCTGGCCCTGCTGGGCGCCCTGCACGCCCAG | |
| | | GCCGAGATGTGGCACGAGGGCCTGGAGGAGGCCAGCAGGCTGT | |
| | | ATTTTGGCGAGCGCAACGTGAAGGGCATGTTCGAGGTGCTGGA | |
| | | GCCTCTGCACGCCATGATGGAGAGAGGCCCACAGACCCTGAAG | |
| | | GAGACATCCTTTAACCAGGCCTATGGACGGGACCTGATGGAGG | |
| | | CACAGGAGTGGTGCAGAAAGTACATGAAGTCTGGCAATGTGAA | |
| | | GGACCTGACGCAGGCCTGGGATCTGTACTATCACGTGTTTCGG | |
| | | AGAATCTCCAAGGGCAAAGACACGATTCCGTGGCTTGGGCATC | |
| | | TGCTCGTTGGGCTGAGTGGTGCGTTTGGTTTCATCATCTTGGT | |
| | | CTATCTCTTGATCAATTGCAGAAATACAGGCCCTTGGCTGAAA | |
| | | AAAGTGCTCAAGTGTAATACCCCCGACCCAAGCAAGTTCTTCT | |
| | | CCCAGCTTTCTTCAGAGCATGGAGGCGATGTGCAGAAATGGCT | |
| | | CTCTTCACCTTTTCCCTCCTCAAGTTTCTCCCCGGGAGGGCTG | |
| | | GCGCCCGAGATTTCACCTCTTGAGGTACTTGAACGAGACAAGG | |
| | | TTACCCAACTTCTCCTTCAACAGGATAAGGTACCCGAACCTGC | |
| | | GAGCCTTAGCTCCAACCACTCTCTTACGAGCTGCTTCACCAAT | |
| | | CAGGGATACTTCTTTTTCCACCTTCCCGATGCGCTGGAAATCG | |
| | | AAGCATGTCAAGTTTACTTTACCTATGATCCATATAGCGAGGA | |
| | | AGATCCCGACGAAGGAGTCGCCGGTGCGCCCACGGGTTCCTCA | |
| | | CCCCAACCTCTCCAGCCTCTCTCAGGAGAAGATGATGCTTATT | |
| | | GCACTTTTCCCAGTAGAGACGATCTCCTCCTCTTTTCTCCATC | |
| | | TCTTTTGGGGGGACCTTCCCCCCCTTCTACGGCACCTGGCGGG | |
| | | TCTGGTGCTGGCGAGGAGCGGATGCCGCCGTCCCTCCAGGAGC | |
| | | GAGTACCACGAGATTGGGATCCCCAGCCACTTGGACCACCAAC | |
| | | TCCAGGCGTACCTGACCTTGTCGATTTTCAACCTCCCCCTGAA | |
| | | TTGGTGCTGCGAGAGGCTGGGGAGGAAGTTCCGGACGCTGGGC | |
| | | CGAGGGAGGGCGTGTCCTTTCCATGGAGTAGGCCTCCAGGTCA | |
| | | AGGCGAGTTTAGGGCTCTCAACGCGCGGCTGCCGTTGAATACA | |
| | | GACGCTTATCTCTCACTGCAGGAACTGCAAGGTCAGGACCCAA | |
| | | CACATCTTGTAGGATCCGGCGCCACCAACTTTAGTCTGCTCAA | |
| | | GCAAGCCGGGGACGTCGAGGAAAATCCTGGGCCAGAAATGTGG | |
| | | CACGAAGGACTCGAGGAAGCCAGTCGGCTGTATTTTGGCGAGC | |
| | | GGAATGTGAAAGGGATGTTTGAAGTGCTCGAGCCTCTCCACGC | |
| | | TATGATGGAACGGGGACCCCAGACTCTCAAAGAAACCAGCTTT | |
| | | AATCAGGCTTACGGACGCGACCTCATGGAAGCTCAAGAATGGT | |
| | | GTAGAAAGTATATGAAGAGTGGCAACGTGAAAGATCTGACACA | |
| | | AGCCTGGGATCTCTATTATCACGTGTTCAGACGCATCAGCAAA | |
| | | GGCAGCGGCGCCACAAATTTCTCCCTGCTGAAACAGGCCGGCG | |
| | | ACGTGGAAGAGAATCCCGGACCTATGCCCAATCCTAGACCTGG | |
| | | CAAGCCCAGCGCTCCTTCTCTTGCTCTTGGACCTTCTCCTGGT | |
| | | GCAAGTCCATCTTGGCGAGCAGCTCCAAAGGCTAGTGATCTTC | |
| | | TCGGAGCTAGAGGTCCTGGAGGCACATTTCAAGGTAGAGACTT | |
| | | GAGGGGAGGTGCTCACGCTTCCTCTTCTTCCTTGAACCCCATG | |
| | | CCACCATCGCAGCTGCAGGTGAGGCCCTGGGCCCAGGATGGGG | |
| | | CAGGCAGGGTGGGGTACCTGGACCTACAGGTGCCGACCTTTAC | |
| | | TGTGGCACTGGGCGGGAGGGGGGCTGGCTGGGGCACAGGAAGT | |
| | | GGTTTCTGGGTCCCAGGCAAGTCTGTGACTTATGCAGATGTTG | |
| | | CAGGGCCAAGAAAATCCCCACCTGCCAGGCCTCAGAGATTGGA | |
| | | GGCTCTCCCCGACCTCCCAATCCCTGTCTCAGGAGAGGAGGAG | |
| | | GCCGTATTGTAGTCCCATGAGCATAGCTATGTGTCCCCATCCC | |
| | | CATGTGACAAGAGAAGAGGACTGGGGCCAAGTAGGTGAGGTGA | |
| | | CAGGGCTGAGGCCAGCTCTGCAACTTATTAGCTGTTTGATCTT | |
| | | TAAAAAGTTACTCGATCTCCATGAGCCTCAGTTTCCATACGTG | |
| | | TAAAAGGGGGATGATCATAGCATCTACCATGTGGGCTTGCAGT | |
| | | GCAGAGTATTTGAATTAGACACAGAACAGTGAGGATCAGGATG | |
| | | GCCTCTCACCCACCTGCCTTTCTGCCCAGCTGCCCACACTGCC | |
| | | CCTAGTCATGGTGGCACCCTCCGGGGCACGGCTGGGCCCCTTG | |
| | | CCCCACTTACAGGCACTCCTCCAG | |
| FoxP3_v6 | 5′ Homology | CACTCACAGCAGCCAGATGAATCTTTTGAGTCCATGCCTAGTC | 197 |
| | Arm | ACTGGGGCAAAATAGGACTCCGAGGAGAAAGTCCGAGACCAGC | |
| | | TCCGGCAAGATGAGCAAACACAGCCTGTGCAGGGTGCAGGGAG | |
| | | GGCTAGAGGCCTGAGGCTTGAAACAGCTCTCAAGTGGAGGGGG | |
| | | AAACAACCATTGCCCTCATAGAGGACACATCCACACCAGGGCT | |
| | | GTGCTAGCGTGGGCAGGCAAGCCAGGTGCTGGACCTCTGCACG | |
| | | TGGGGCATGTGTGGGTATGTACATGTACCTGTGTTCTTGGTGT | |
| | | GTGTGTGTGTGTGTGTGTGTGTGTGTGTCTAGAGCTGGGGTGC | |
| | | AACTATGGGGCCCCTCGGGACATGTCCCAGCCAATGCCTGCTT | |
| | | TGACCAGAGGAGTGTCCACGTGGCTCAGGTGGTCGAGTATCTC | |
| | | ATACCGCCCTAGCACACGTGTGACTCCTTTCCCCTATTGTCTA | |
| | | CGCAGCCTGCCCTTGGACAAGGACCCGATGCCCAACCCCAGGC | |
| | | CTGGCAAGCCCTCGGCCCCTTCCTTGGCCCTTGGCCCATCCCC | |
| | | AGGAGCCTCGCCCAGCTGGAGGGCTGCACCCAAAGCCTCAGAC | |
| | | CTGCTGGGGGCCCGGGGCCCAGGGGGAACCTTCCAGGGCCGAG | |
| | | ATCTT | |
| | Intervening | AGGT | |
| | sequence | | |
| | MND Promoter | GAACAGAGAAACAGGAGAATATGGGCCAAACAGGATATCTGTG | 198 |
| | | GTAAGCAGTTCCTGCCCCGGCTCAGGGCCAAGAACAGTTGGAA | |
| | | CAGCAGAATATGGGCCAAACAGGATATCTGTGGTAAGCAGTTC | |
| | | CTGCCCCGGCTCAGGGCCAAGAACAGATGGTCCCCAGATGCGG | |
| | | TCCCGCCCTCAGCAGTTTCTAGAGAACCATCAGATGTTTCCAG | |
| | | GGTGCCCCAAGGACCTGAAATGACCCTGTGCCTTATTTGAACT | |
| | | AACCAATCAGTTCGCTTCTCGCTTCTGTTCGCGCGCTTCTGCT | |
| | | CCCCGAGCTCTATATAAGCAGAGCTCGTTTAGTGAACCGTCAG | |
| | | ATC | |
| | Intervening | GCCTGGAGACGCCATCCACGCTGTTTTGACTTCCATAGAAGGA | 199 |
| | Sequence | TCTCGAGGCCACCATGCCTCTGGGCCTGCTGTGGCTGGGCCTG | |
| | (LCN2 signal | GCCCTGCTGGGCGCCCTGCACGCCCAGGCC | |
| | peptide of | | |
| | FRB-IL2Rb | | |
| | underlined) | | |
| | FRB-IL2Rb | GAGATGTGGCACGAGGGCCTGGAGGAGGCCAGCAGGCTGTATT | 200 |
| | | TTGGCGAGCGCAACGTGAAGGGCATGTTCGAGGTGCTGGAGCC | |
| | | TCTGCACGCCATGATGGAGAGAGGCCCACAGACCCTGAAGGAG | |
| | | ACATCCTTTAACCAGGCCTATGGACGGGACCTGATGGAGGCAC | |
| | | AGGAGTGGTGCAGAAAGTACATGAAGTCTGGCAATGTGAAGGA | |
| | | CCTGACGCAGGCCTGGGATCTGTACTATCACGTGTTTCGGAGA | |
| | | ATCTCCAAGGGCAAAGACACGATTCCGTGGCTTGGGCATCTGC | |
| | | TCGTTGGGCTGAGTGGTGCGTTTGGTTTCATCATCTTGGTCTA | |
| | | TCTCTTGATCAATTGCAGAAATACAGGCCCTTGGCTGAAAAAA | |
| | | GTGCTCAAGTGTAATACCCCCGACCCAAGCAAGTTCTTCTCCC | |
| | | AGCTTTCTTCAGAGCATGGAGGCGATGTGCAGAAATGGCTCTC | |
| | | TTCACCTTTTCCCTCCTCAAGTTTCTCCCCGGGAGGGCTGGCG | |
| | | CCCGAGATTTCACCTCTTGAGGTACTTGAACGAGACAAGGTTA | |
| | | CCCAACTTCTCCTTCAACAGGATAAGGTACCCGAACCTGCGAG | |
| | | CCTTAGCTCCAACCACTCTCTTACGAGCTGCTTCACCAATCAG | |
| | | GGATACTTCTTTTTCCACCTTCCCGATGCGCTGGAAATCGAAG | |
| | | CATGTCAAGTTTACTTTACCTATGATCCATATAGCGAGGAAGA | |
| | | TCCCGACGAAGGAGTCGCCGGTGCGCCCACGGGTTCCTCACCC | |
| | | CAACCTCTCCAGCCTCTCTCAGGAGAAGATGATGCTTATTGCA | |
| | | CTTTTCCCAGTAGAGACGATCTCCTCCTCTTTTCTCCATCTCT | |
| | | TTTGGGGGGACCTTCCCCCCCTTCTACGGCACCTGGCGGGTCT | |
| | | GGTGCTGGCGAGGAGCGGATGCCGCCGTCCCTCCAGGAGCGAG | |
| | | TACCACGAGATTGGGATCCCCAGCCACTTGGACCACCAACTCC | |
| | | AGGCGTACCTGACCTTGTCGATTTTCAACCTCCCCCTGAATTG | |
| | | GTGCTGCGAGAGGCTGGGGAGGAAGTTCCGGACGCTGGGCCGA | |
| | | GGGAGGGCGTGTCCTTTCCATGGAGTAGGCCTCCAGGTCAAGG | |
| | | CGAGTTTAGGGCTCTCAACGCGCGGCTGCCGTTGAATACAGAC | |
| | | GCTTATCTCTCACTGCAGGAACTGCAAGGTCAGGACCCAACAC | |
| | | ATCTTGTA | |
| | GSG linker | GGATCCGGC | |
| | P2A | GCCACCAACTTTAGTCTGCTCAAGCAAGCCGGGGACGTCGAGG | 201 |
| | | AAAATCCTGGGCCA | |
| | Cytosolic | GAAATGTGGCACGAAGGACTCGAGGAAGCCAGTCGGCTGTATT | 202 |
| | FRB | TTGGCGAGCGGAATGTGAAAGGGATGTTTGAAGTGCTCGAGCC | |
| | | TCTCCACGCTATGATGGAACGGGGACCCCAGACTCTCAAAGAA | |
| | | ACCAGCTTTAATCAGGCTTACGGACGCGACCTCATGGAAGCTC | |
| | | AAGAATGGTGTAGAAAGTATATGAAGAGTGGCAACGTGAAAGA | |
| | | TCTGACACAAGCCTGGGATCTCTATTATCACGTGTTCAGACGC | |
| | | ATCAGCAAA | |
| | GSG linker | GGCAGCGGC | |
| | Second P2A | GCCACAAATTTCTCCCTGCTGAAACAGGCCGGCGACGTGGAAG | 203 |
| | | AGAATCCCGGACCT | |
| | FoxP3 | ATGCCCAATCCTAGACCTGGCAAGCCCAGCGCTCCTTCTCTTG | 204 |
| | portion | CTCTTGGACCTTCTCCTGGTGCAAGTCCATCTTGGCGAGCAGC | |
| | | TCCAAAGGCTAGTGATCTTCTCGGAGCTAGAGGTCCTGGAGGC | |
| | | ACATTTCAAGGTAGGGACTTGCGTGGAGGT | |
| | 3′ Homology | GCCCATGCCTCCTCTTCTTCCTTGAACCCCATGCCACCATCGC | 205 |
| | Arm | AGCTGCAGGTGAGGCCCTGGGCCCAGGATGGGGCAGGCAGGGT | |
| | | GGGGTACCTGGACCTACAGGTGCCGACCTTTACTGTGGCACTG | |
| | | GGCGGGAGGGGGGCTGGCTGGGGCACAGGAAGTGGTTTCTGGG | |
| | | TCCCAGGCAAGTCTGTGACTTATGCAGATGTTGCAGGGCCAAG | |
| | | AAAATCCCCACCTGCCAGGCCTCAGAGATTGGAGGCTCTCCCC | |
| | | GACCTCCCAATCCCTGTCTCAGGAGAGGAGGAGGCCGTATTGT | |
| | | AGTCCCATGAGCATAGCTATGTGTCCCCATCCCCATGTGACAA | |
| | | GAGAAGAGGACTGGGGCCAAGTAGGTGAGGTGACAGGGCTGAG | |
| | | GCCAGCTCTGCAACTTATTAGCTGTTTGATCTTTAAAAAGTTA | |
| | | CTCGATCTCCATGAGCCTCAGTTTCCATACGTGTAAAAGGGGG | |
| | | ATGATCATAGCATCTACCATGTGGGCTTGCAGTGCAGAGTATT | |
| | | TGAATTAGACACAGAACAGTGAGGATCAGGATGGCCTCTCACC | |
| | | CACCTGCCTTTCTGCCCAGCTGCCCACACTGCCCCTAGTCATG | |
| | | GTGGCACCCTCCGGGGCACGGCTGGGCCCCTTGCCCCACTTAC | |
| | | AGGCA | |
| | Between | AGGTGAACAGAGAAACAGGAGAATATGGGCCAAACAGGATATC | 206 |
| | Homology | TGTGGTAAGCAGTTCCTGCCCCGGCTCAGGGCCAAGAACAGTT | |
| | Arms | GGAACAGCAGAATATGGGCCAAACAGGATATCTGTGGTAAGCA | |
| | | GTTCCTGCCCCGGCTCAGGGCCAAGAACAGATGGTCCCCAGAT | |
| | | GCGGTCCCGCCCTCAGCAGTTTCTAGAGAACCATCAGATGTTT | |
| | | CCAGGGTGCCCCAAGGACCTGAAATGACCCTGTGCCTTATTTG | |
| | | AACTAACCAATCAGTTCGCTTCTCGCTTCTGTTCGCGCGCTTC | |
| | | TGCTCCCCGAGCTCTATATAAGCAGAGCTCGTTTAGTGAACCG | |
| | | TCAGATCGCCTGGAGACGCCATCCACGCTGTTTTGACTTCCAT | |
| | | AGAAGGATCTCGAGGCCACCATGCCTCTGGGCCTGCTGTGGCT | |
| | | GGGCCTGGCCCTGCTGGGCGCCCTGCACGCCCAGGCCGAGATG | |
| | | TGGCACGAGGGCCTGGAGGAGGCCAGCAGGCTGTATTTTGGCG | |
| | | AGCGCAACGTGAAGGGCATGTTCGAGGTGCTGGAGCCTCTGCA | |
| | | CGCCATGATGGAGAGAGGCCCACAGACCCTGAAGGAGACATCC | |
| | | TTTAACCAGGCCTATGGACGGGACCTGATGGAGGCACAGGAGT | |
| | | GGTGCAGAAAGTACATGAAGTCTGGCAATGTGAAGGACCTGAC | |
| | | GCAGGCCTGGGATCTGTACTATCACGTGTTTCGGAGAATCTCC | |
| | | AAGGGCAAAGACACGATTCCGTGGCTTGGGCATCTGCTCGTTG | |
| | | GGCTGAGTGGTGCGTTTGGTTTCATCATCTTGGTCTATCTCTT | |
| | | GATCAATTGCAGAAATACAGGCCCTTGGCTGAAAAAAGTGCTC | |
| | | AAGTGTAATACCCCCGACCCAAGCAAGTTCTTCTCCCAGCTTT | |
| | | CTTCAGAGCATGGAGGCGATGTGCAGAAATGGCTCTCTTCACC | |
| | | TTTTCCCTCCTCAAGTTTCTCCCCGGGAGGGCTGGCGCCCGAG | |
| | | ATTTCACCTCTTGAGGTACTTGAACGAGACAAGGTTACCCAAC | |
| | | TTCTCCTTCAACAGGATAAGGTACCCGAACCTGCGAGCCTTAG | |
| | | CTCCAACCACTCTCTTACGAGCTGCTTCACCAATCAGGGATAC | |
| | | TTCTTTTTCCACCTTCCCGATGCGCTGGAAATCGAAGCATGTC | |
| | | AAGTTTACTTTACCTATGATCCATATAGCGAGGAAGATCCCGA | |
| | | CGAAGGAGTCGCCGGTGCGCCCACGGGTTCCTCACCCCAACCT | |
| | | CTCCAGCCTCTCTCAGGAGAAGATGATGCTTATTGCACTTTTC | |
| | | CCAGTAGAGACGATCTCCTCCTCTTTTCTCCATCTCTTTTGGG | |
| | | GGGACCTTCCCCCCCTTCTACGGCACCTGGCGGGTCTGGTGCT | |
| | | GGCGAGGAGCGGATGCCGCCGTCCCTCCAGGAGCGAGTACCAC | |
| | | GAGATTGGGATCCCCAGCCACTTGGACCACCAACTCCAGGCGT | |
| | | ACCTGACCTTGTCGATTTTCAACCTCCCCCTGAATTGGTGCTG | |
| | | CGAGAGGCTGGGGAGGAAGTTCCGGACGCTGGGCCGAGGGAGG | |
| | | GCGTGTCCTTTCCATGGAGTAGGCCTCCAGGTCAAGGCGAGTT | |
| | | TAGGGCTCTCAACGCGCGGCTGCCGTTGAATACAGACGCTTAT | |
| | | CTCTCACTGCAGGAACTGCAAGGTCAGGACCCAACACATCTTG | |
| | | TAGGATCCGGCGCCACCAACTTTAGTCTGCTCAAGCAAGCCGG | |
| | | GGACGTCGAGGAAAATCCTGGGCCAGAAATGTGGCACGAAGGA | |
| | | CTCGAGGAAGCCAGTCGGCTGTATTTTGGCGAGCGGAATGTGA | |
| | | AAGGGATGTTTGAAGTGCTCGAGCCTCTCCACGCTATGATGGA | |
| | | ACGGGGACCCCAGACTCTCAAAGAAACCAGCTTTAATCAGGCT | |
| | | TACGGACGCGACCTCATGGAAGCTCAAGAATGGTGTAGAAAGT | |
| | | ATATGAAGAGTGGCAACGTGAAAGATCTGACACAAGCCTGGGA | |
| | | TCTCTATTATCACGTGTTCAGACGCATCAGCAAAGGCAGCGGC | |
| | | GCCACAAATTTCTCCCTGCTGAAACAGGCCGGCGACGTGGAAG | |
| | | AGAATCCCGGACCTATGCCCAATCCTAGACCTGGCAAGCCCAG | |
| | | CGCTCCTTCTCTTGCTCTTGGACCTTCTCCTGGTGCAAGTCCA | |
| | | TCTTGGCGAGCAGCTCCAAAGGCTAGTGATCTTCTCGGAGCTA | |
| | | GAGGTCCTGGAGGCACATTTCAAGGTAGGGACTTGCGTGGAGG | |
| | | T | |
| | Full-length | CACTCACAGCAGCCAGATGAATCTTTTGAGTCCATGCCTAGTC | 207 |
| | donor | ACTGGGGCAAAATAGGACTCCGAGGAGAAAGTCCGAGACCAGC | |
| | template | TCCGGCAAGATGAGCAAACACAGCCTGTGCAGGGTGCAGGGAG | |
| | | GGCTAGAGGCCTGAGGCTTGAAACAGCTCTCAAGTGGAGGGGG | |
| | | AAACAACCATTGCCCTCATAGAGGACACATCCACACCAGGGCT | |
| | | GTGCTAGCGTGGGCAGGCAAGCCAGGTGCTGGACCTCTGCACG | |
| | | TGGGGCATGTGTGGGTATGTACATGTACCTGTGTTCTTGGTGT | |
| | | GTGTGTGTGTGTGTGTGTGTGTGTGTGTCTAGAGCTGGGGTGC | |
| | | AACTATGGGGCCCCTCGGGACATGTCCCAGCCAATGCCTGCTT | |
| | | TGACCAGAGGAGTGTCCACGTGGCTCAGGTGGTCGAGTATCTC | |
| | | ATACCGCCCTAGCACACGTGTGACTCCTTTCCCCTATTGTCTA | |
| | | CGCAGCCTGCCCTTGGACAAGGACCCGATGCCCAACCCCAGGC | |
| | | CTGGCAAGCCCTCGGCCCCTTCCTTGGCCCTTGGCCCATCCCC | |
| | | AGGAGCCTCGCCCAGCTGGAGGGCTGCACCCAAAGCCTCAGAC | |
| | | CTGCTGGGGGCCCGGGGCCCAGGGGGAACCTTCCAGGGCCGAG | |
| | | ATCTTAGGTGAACAGAGAAACAGGAGAATATGGGCCAAACAGG | |
| | | ATATCTGTGGTAAGCAGTTCCTGCCCCGGCTCAGGGCCAAGAA | |
| | | CAGTTGGAACAGCAGAATATGGGCCAAACAGGATATCTGTGGT | |
| | | AAGCAGTTCCTGCCCCGGCTCAGGGCCAAGAACAGATGGTCCC | |
| | | CAGATGCGGTCCCGCCCTCAGCAGTTTCTAGAGAACCATCAGA | |
| | | TGTTTCCAGGGTGCCCCAAGGACCTGAAATGACCCTGTGCCTT | |
| | | ATTTGAACTAACCAATCAGTTCGCTTCTCGCTTCTGTTCGCGC | |
| | | GCTTCTGCTCCCCGAGCTCTATATAAGCAGAGCTCGTTTAGTG | |
| | | AACCGTCAGATCGCCTGGAGACGCCATCCACGCTGTTTTGACT | |
| | | TCCATAGAAGGATCTCGAGGCCACCATGCCTCTGGGCCTGCTG | |
| | | TGGCTGGGCCTGGCCCTGCTGGGCGCCCTGCACGCCCAGGCCG | |
| | | AGATGTGGCACGAGGGCCTGGAGGAGGCCAGCAGGCTGTATTT | |
| | | TGGCGAGCGCAACGTGAAGGGCATGTTCGAGGTGCTGGAGCCT | |
| | | CTGCACGCCATGATGGAGAGAGGCCCACAGACCCTGAAGGAGA | |
| | | CATCCTTTAACCAGGCCTATGGACGGGACCTGATGGAGGCACA | |
| | | GGAGTGGTGCAGAAAGTACATGAAGTCTGGCAATGTGAAGGAC | |
| | | CTGACGCAGGCCTGGGATCTGTACTATCACGTGTTTCGGAGAA | |
| | | TCTCCAAGGGCAAAGACACGATTCCGTGGCTTGGGCATCTGCT | |
| | | CGTTGGGCTGAGTGGTGCGTTTGGTTTCATCATCTTGGTCTAT | |
| | | CTCTTGATCAATTGCAGAAATACAGGCCCTTGGCTGAAAAAAG | |
| | | TGCTCAAGTGTAATACCCCCGACCCAAGCAAGTTCTTCTCCCA | |
| | | GCTTTCTTCAGAGCATGGAGGCGATGTGCAGAAATGGCTCTCT | |
| | | TCACCTTTTCCCTCCTCAAGTTTCTCCCCGGGAGGGCTGGCGC | |
| | | CCGAGATTTCACCTCTTGAGGTACTTGAACGAGACAAGGTTAC | |
| | | CCAACTTCTCCTTCAACAGGATAAGGTACCCGAACCTGCGAGC | |
| | | CTTAGCTCCAACCACTCTCTTACGAGCTGCTTCACCAATCAGG | |
| | | GATACTTCTTTTTCCACCTTCCCGATGCGCTGGAAATCGAAGC | |
| | | ATGTCAAGTTTACTTTACCTATGATCCATATAGCGAGGAAGAT | |
| | | CCCGACGAAGGAGTCGCCGGTGCGCCCACGGGTTCCTCACCCC | |
| | | AACCTCTCCAGCCTCTCTCAGGAGAAGATGATGCTTATTGCAC | |
| | | TTTTCCCAGTAGAGACGATCTCCTCCTCTTTTCTCCATCTCTT | |
| | | TTGGGGGGACCTTCCCCCCCTTCTACGGCACCTGGCGGGTCTG | |
| | | GTGCTGGCGAGGAGCGGATGCCGCCGTCCCTCCAGGAGCGAGT | |
| | | ACCACGAGATTGGGATCCCCAGCCACTTGGACCACCAACTCCA | |
| | | GGCGTACCTGACCTTGTCGATTTTCAACCTCCCCCTGAATTGG | |
| | | TGCTGCGAGAGGCTGGGGAGGAAGTTCCGGACGCTGGGCCGAG | |
| | | GGAGGGCGTGTCCTTTCCATGGAGTAGGCCTCCAGGTCAAGGC | |
| | | GAGTTTAGGGCTCTCAACGCGCGGCTGCCGTTGAATACAGACG | |
| | | CTTATCTCTCACTGCAGGAACTGCAAGGTCAGGACCCAACACA | |
| | | TCTTGTAGGATCCGGCGCCACCAACTTTAGTCTGCTCAAGCAA | |
| | | GCCGGGGACGTCGAGGAAAATCCTGGGCCAGAAATGTGGCACG | |
| | | AAGGACTCGAGGAAGCCAGTCGGCTGTATTTTGGCGAGCGGAA | |
| | | TGTGAAAGGGATGTTTGAAGTGCTCGAGCCTCTCCACGCTATG | |
| | | ATGGAACGGGGACCCCAGACTCTCAAAGAAACCAGCTTTAATC | |
| | | AGGCTTACGGACGCGACCTCATGGAAGCTCAAGAATGGTGTAG | |
| | | AAAGTATATGAAGAGTGGCAACGTGAAAGATCTGACACAAGCC | |
| | | TGGGATCTCTATTATCACGTGTTCAGACGCATCAGCAAAGGCA | |
| | | GCGGCGCCACAAATTTCTCCCTGCTGAAACAGGCCGGCGACGT | |
| | | GGAAGAGAATCCCGGACCTATGCCCAATCCTAGACCTGGCAAG | |
| | | CCCAGCGCTCCTTCTCTTGCTCTTGGACCTTCTCCTGGTGCAA | |
| | | GTCCATCTTGGCGAGCAGCTCCAAAGGCTAGTGATCTTCTCGG | |
| | | AGCTAGAGGTCCTGGAGGCACATTTCAAGGTAGGGACTTGCGT | |
| | | GGAGGTGCCCATGCCTCCTCTTCTTCCTTGAACCCCATGCCAC | |
| | | CATCGCAGCTGCAGGTGAGGCCCTGGGCCCAGGATGGGGCAGG | |
| | | CAGGGTGGGGTACCTGGACCTACAGGTGCCGACCTTTACTGTG | |
| | | GCACTGGGCGGGAGGGGGGCTGGCTGGGGCACAGGAAGTGGTT | |
| | | TCTGGGTCCCAGGCAAGTCTGTGACTTATGCAGATGTTGCAGG | |
| | | GCCAAGAAAATCCCCACCTGCCAGGCCTCAGAGATTGGAGGCT | |
| | | CTCCCCGACCTCCCAATCCCTGTCTCAGGAGAGGAGGAGGCCG | |
| | | TATTGTAGTCCCATGAGCATAGCTATGTGTCCCCATCCCCATG | |
| | | TGACAAGAGAAGAGGACTGGGGCCAAGTAGGTGAGGTGACAGG | |
| | | GCTGAGGCCAGCTCTGCAACTTATTAGCTGTTTGATCTTTAAA | |
| | | AAGTTACTCGATCTCCATGAGCCTCAGTTTCCATACGTGTAAA | |
| | | AGGGGGATGATCATAGCATCTACCATGTGGGCTTGCAGTGCAG | |
| | | AGTATTTGAATTAGACACAGAACAGTGAGGATCAGGATGGCCT | |
| | | CTCACCCACCTGCCTTTCTGCCCAGCTGCCCACACTGCCCCTA | |
| | | GTCATGGTGGCACCCTCCGGGGCACGGCTGGGCCCCTTGCCCC | |
| | | ACTTACAGGCA | |
| FoxP3_v7 | 5′ Homology | ACTTGCCAGGACTGTTACAATAGCCTCCTCACTAGCCCCACTC | 208 |
| | Arm | ACAGCAGCCAGATGAATCTTTTGAGTCCATGCCTAGTCACTGG | |
| | | GGCAAAATAGGACTCCGAGGAGAAAGTCCGAGACCAGCTCCGG | |
| | | CAAGATGAGCAAACACAGCCTGTGCAGGGTGCAGGGAGGGCTA | |
| | | GAGGCCTGAGGCTTGAAACAGCTCTCAAGTGGAGGGGGAAACA | |
| | | ACCATTGCCCTCATAGAGGACACATCCACACCAGGGCTGTGCT | |
| | | AGCGTGGGCAGGCAAGCCAGGTGCTGGACCTCTGCACGTGGGG | |
| | | CATGTGTGGGTATGTACATGTACCTGTGTTCTTGGTGTGTGTG | |
| | | TGTGTGTGTGTGTGTGTGTGTGTCTAGAGCTGGGGTGCAACTA | |
| | | TGGGGCCCCTCGGGACATGTCCCAGCCAATGCCTGCTTTGACC | |
| | | AGAGGAGTGTCCACGTGGCTCAGGTGGTCGAGTATCTCATACC | |
| | | GCCCTAGCACACGTGTGACTCCTTTCCCCTATTGTCTACGCAG | |
| | | CCTGCCCTTGGACAAGGACCCGATGCCCAACCCCAGGCCTGGC | |
| | | AAGCCCTCGGCCCCTTCCTTGGCCCTTGGCCCATCCCCAGGAG | |
| | | CCTCGCCCAGCTGGAGGGCTGCACCCAAAGCCTCAGACCTGCT | |
| | | GGGGGCCCGGGG | |
| | Intervening | CCCAGGGGGAACCTTCCAGT | 209 |
| | sequence | | |
| | MND Promoter | GAACAGAGAAACAGGAGAATATGGGCCAAACAGGATATCTGTG | 210 |
| | | GTAAGCAGTTCCTGCCCCGGCTCAGGGCCAAGAACAGTTGGAA | |
| | | CAGCAGAATATGGGCCAAACAGGATATCTGTGGTAAGCAGTTC | |
| | | CTGCCCCGGCTCAGGGCCAAGAACAGATGGTCCCCAGATGCGG | |
| | | TCCCGCCCTCAGCAGTTTCTAGAGAACCATCAGATGTTTCCAG | |
| | | GGTGCCCCAAGGACCTGAAATGACCCTGTGCCTTATTTGAACT | |
| | | AACCAATCAGTTCGCTTCTCGCTTCTGTTCGCGCGCTTCTGCT | |
| | | CCCCGAGCTCTATATAAGCAGAGCTCGTTTAGTGAACCGTCAG | |
| | | ATC | |
| | Intervening | GCCTGGAGACGCCATCCACGCTGTTTTGACTTCCATAGAAGGA | 211 |
| | Sequence | TCTCGCCGCCACCATGCCTCTGGGCCTGCTGTGGCTGGGCCTG | |
| | (LCN2 signal | GCCCTGCTGGGCGCCCTGCACGCCCAGGCC | |
| | peptide of | | |
| | FRB-IL2Rb | | |
| | underlined) | | |
| | FRB-IL2Rb | GAGATGTGGCACGAGGGCCTGGAGGAGGCCAGCAGGCTGTATT | 212 |
| | | TTGGCGAGCGCAACGTGAAGGGCATGTTCGAGGTGCTGGAGCC | |
| | | TCTGCACGCCATGATGGAGAGAGGCCCACAGACCCTGAAGGAG | |
| | | ACATCCTTTAACCAGGCCTATGGACGGGACCTGATGGAGGCAC | |
| | | AGGAGTGGTGCAGAAAGTACATGAAGTCTGGCAATGTGAAGGA | |
| | | CCTGACGCAGGCCTGGGATCTGTACTATCACGTGTTTCGGAGA | |
| | | ATCTCCAAGGGCAAAGACACGATTCCGTGGCTTGGGCATCTGC | |
| | | TCGTTGGGCTGAGTGGTGCGTTTGGTTTCATCATCTTGGTCTA | |
| | | TCTCTTGATCAATTGCAGAAATACAGGCCCTTGGCTGAAAAAA | |
| | | GTGCTCAAGTGTAATACCCCCGACCCAAGCAAGTTCTTCTCCC | |
| | | AGCTTTCTTCAGAGCATGGAGGCGATGTGCAGAAATGGCTCTC | |
| | | TTCACCTTTTCCCTCCTCAAGTTTCTCCCCGGGAGGGCTGGCG | |
| | | CCCGAGATTTCACCTCTTGAGGTACTTGAACGAGACAAGGTTA | |
| | | CCCAACTTCTCCTTCAACAGGATAAGGTACCCGAACCTGCGAG | |
| | | CCTTAGCTCCAACCACTCTCTTACGAGCTGCTTCACCAATCAG | |
| | | GGATACTTCTTTTTCCACCTTCCCGATGCGCTGGAAATCGAAG | |
| | | CATGTCAAGTTTACTTTACCTATGATCCATATAGCGAGGAAGA | |
| | | TCCCGACGAAGGAGTCGCCGGTGCGCCCACGGGTTCCTCACCC | |
| | | CAACCTCTCCAGCCTCTCTCAGGAGAAGATGATGCTTATTGCA | |
| | | CTTTTCCCAGTAGAGACGATCTCCTCCTCTTTTCTCCATCTCT | |
| | | TTTGGGGGGACCTTCCCCCCCTTCTACGGCACCTGGCGGGTCT | |
| | | GGTGCTGGCGAGGAGCGGATGCCGCCGTCCCTCCAGGAGCGAG | |
| | | TACCACGAGATTGGGATCCCCAGCCACTTGGACCACCAACTCC | |
| | | AGGCGTACCTGACCTTGTCGATTTTCAACCTCCCCCTGAATTG | |
| | | GTGCTGCGAGAGGCTGGGGAGGAAGTTCCGGACGCTGGGCCGA | |
| | | GGGAGGGCGTGTCCTTTCCATGGAGTAGGCCTCCAGGTCAAGG | |
| | | CGAGTTTAGGGCTCTCAACGCGCGGCTGCCGTTGAATACAGAC | |
| | | GCTTATCTCTCACTGCAGGAACTGCAAGGTCAGGACCCAACAC | |
| | | ATCTTGTA | |
| | GSG linker | GGATCCGGC | |
| | P2A | GCCACCAACTTTAGTCTGCTCAAGCAAGCCGGGGACGTCGAGG | 213 |
| | | AAAATCCTGGGCCA | |
| | Cytosolic | GAAATGTGGCACGAAGGACTCGAGGAAGCCAGTCGGCTGTATT | 214 |
| | FRB | TTGGCGAGCGGAATGTGAAAGGGATGTTTGAAGTGCTCGAGCC | |
| | | TCTCCACGCTATGATGGAACGGGGACCCCAGACTCTCAAAGAA | |
| | | ACCAGCTTTAATCAGGCTTACGGACGCGACCTCATGGAAGCTC | |
| | | AAGAATGGTGTAGAAAGTATATGAAGAGTGGCAACGTGAAAGA | |
| | | TCTGACACAAGCCTGGGATCTCTATTATCACGTGTTCAGACGC | |
| | | ATCAGCAAA | |
| | GSG linker | GGCAGCGGC | |
| | Second P2A | GCCACAAATTTCTCCCTGCTGAAACAGGCCGGCGACGTGGAAG | 215 |
| | | AGAATCCCGGACCT | |
| | FoxP3 | ATGCCCAATCCTAGACCTGGCAAGCCCAGCGCTCCTTCTCTTG | 216 |
| | portion | CTCTTGGACCTTCTCCTGGTGCAAGTCCATCTTGGCGAGCAGC | |
| | | TCCAAAGGCTAGTGATCTTCTCGGAGCTAGAGGTCCTGGAGGC | |
| | | ACATTTGGA | |
| | 3′ Homology | CGAGATCTTCGAGGCGGGGCCCATGCCTCCTCTTCTTCCTTGA | 217 |
| | Arm | ACCCCATGCCACCATCGCAGCTGCAGGTGAGGCCCTGGGCCCA | |
| | | GGATGGGGCAGGCAGGGTGGGGTACCTGGACCTACAGGTGCCG | |
| | | ACCTTTACTGTGGCACTGGGCGGGAGGGGGGCTGGCTGGGGCA | |
| | | CAGGAAGTGGTTTCTGGGTCCCAGGCAAGTCTGTGACTTATGC | |
| | | AGATGTTGCAGGGCCAAGAAAATCCCCACCTGCCAGGCCTCAG | |
| | | AGATTGGAGGCTCTCCCCGACCTCCCAATCCCTGTCTCAGGAG | |
| | | AGGAGGAGGCCGTATTGTAGTCCCATGAGCATAGCTATGTGTC | |
| | | CCCATCCCCATGTGACAAGAGAAGAGGACTGGGGCCAAGTAGG | |
| | | TGAGGTGACAGGGCTGAGGCCAGCTCTGCAACTTATTAGCTGT | |
| | | TTGATCTTTAAAAAGTTACTCGATCTCCATGAGCCTCAGTTTC | |
| | | CATACGTGTAAAAGGGGGATGATCATAGCATCTACCATGTGGG | |
| | | CTTGCAGTGCAGAGTATTTGAATTAGACACAGAACAGTGAGGA | |
| | | TCAGGATGGCCTCTCACCCACCTGCCTTTCTGCCCAGCTGCCC | |
| | | ACACTGCCCCTAGTCATGGTGGCACCCTCCGGGGCACGGCTGG | |
| | | GCCCCT | |
| | Between | CCCAGGGGGAACCTTCCAGTGAACAGAGAAACAGGAGAATATG | 218 |
| | Homology | GGCCAAACAGGATATCTGTGGTAAGCAGTTCCTGCCCCGGCTC | |
| | Arms | AGGGCCAAGAACAGTTGGAACAGCAGAATATGGGCCAAACAGG | |
| | | ATATCTGTGGTAAGCAGTTCCTGCCCCGGCTCAGGGCCAAGAA | |
| | | CAGATGGTCCCCAGATGCGGTCCCGCCCTCAGCAGTTTCTAGA | |
| | | GAACCATCAGATGTTTCCAGGGTGCCCCAAGGACCTGAAATGA | |
| | | CCCTGTGCCTTATTTGAACTAACCAATCAGTTCGCTTCTCGCT | |
| | | TCTGTTCGCGCGCTTCTGCTCCCCGAGCTCTATATAAGCAGAG | |
| | | CTCGTTTAGTGAACCGTCAGATCGCCTGGAGACGCCATCCACG | |
| | | CTGTTTTGACTTCCATAGAAGGATCTCGCCGCCACCATGCCTC | |
| | | TGGGCCTGCTGTGGCTGGGCCTGGCCCTGCTGGGCGCCCTGCA | |
| | | CGCCCAGGCCGAGATGTGGCACGAGGGCCTGGAGGAGGCCAGC | |
| | | AGGCTGTATTTTGGCGAGCGCAACGTGAAGGGCATGTTCGAGG | |
| | | TGCTGGAGCCTCTGCACGCCATGATGGAGAGAGGCCCACAGAC | |
| | | CCTGAAGGAGACATCCTTTAACCAGGCCTATGGACGGGACCTG | |
| | | ATGGAGGCACAGGAGTGGTGCAGAAAGTACATGAAGTCTGGCA | |
| | | ATGTGAAGGACCTGACGCAGGCCTGGGATCTGTACTATCACGT | |
| | | GTTTCGGAGAATCTCCAAGGGCAAAGACACGATTCCGTGGCTT | |
| | | GGGCATCTGCTCGTTGGGCTGAGTGGTGCGTTTGGTTTCATCA | |
| | | TCTTGGTCTATCTCTTGATCAATTGCAGAAATACAGGCCCTTG | |
| | | GCTGAAAAAAGTGCTCAAGTGTAATACCCCCGACCCAAGCAAG | |
| | | TTCTTCTCCCAGCTTTCTTCAGAGCATGGAGGCGATGTGCAGA | |
| | | AATGGCTCTCTTCACCTTTTCCCTCCTCAAGTTTCTCCCCGGG | |
| | | AGGGCTGGCGCCCGAGATTTCACCTCTTGAGGTACTTGAACGA | |
| | | GACAAGGTTACCCAACTTCTCCTTCAACAGGATAAGGTACCCG | |
| | | AACCTGCGAGCCTTAGCTCCAACCACTCTCTTACGAGCTGCTT | |
| | | CACCAATCAGGGATACTTCTTTTTCCACCTTCCCGATGCGCTG | |
| | | GAAATCGAAGCATGTCAAGTTTACTTTACCTATGATCCATATA | |
| | | GCGAGGAAGATCCCGACGAAGGAGTCGCCGGTGCGCCCACGGG | |
| | | TTCCTCACCCCAACCTCTCCAGCCTCTCTCAGGAGAAGATGAT | |
| | | GCTTATTGCACTTTTCCCAGTAGAGACGATCTCCTCCTCTTTT | |
| | | CTCCATCTCTTTTGGGGGGACCTTCCCCCCCTTCTACGGCACC | |
| | | TGGCGGGTCTGGTGCTGGCGAGGAGCGGATGCCGCCGTCCCTC | |
| | | CAGGAGCGAGTACCACGAGATTGGGATCCCCAGCCACTTGGAC | |
| | | CACCAACTCCAGGCGTACCTGACCTTGTCGATTTTCAACCTCC | |
| | | CCCTGAATTGGTGCTGCGAGAGGCTGGGGAGGAAGTTCCGGAC | |
| | | GCTGGGCCGAGGGAGGGCGTGTCCTTTCCATGGAGTAGGCCTC | |
| | | CAGGTCAAGGCGAGTTTAGGGCTCTCAACGCGCGGCTGCCGTT | |
| | | GAATACAGACGCTTATCTCTCACTGCAGGAACTGCAAGGTCAG | |
| | | GACCCAACACATCTTGTAGGATCCGGCGCCACCAACTTTAGTC | |
| | | TGCTCAAGCAAGCCGGGGACGTCGAGGAAAATCCTGGGCCAGA | |
| | | AATGTGGCACGAAGGACTCGAGGAAGCCAGTCGGCTGTATTTT | |
| | | GGCGAGCGGAATGTGAAAGGGATGTTTGAAGTGCTCGAGCCTC | |
| | | TCCACGCTATGATGGAACGGGGACCCCAGACTCTCAAAGAAAC | |
| | | CAGCTTTAATCAGGCTTACGGACGCGACCTCATGGAAGCTCAA | |
| | | GAATGGTGTAGAAAGTATATGAAGAGTGGCAACGTGAAAGATC | |
| | | TGACACAAGCCTGGGATCTCTATTATCACGTGTTCAGACGCAT | |
| | | CAGCAAAGGCAGCGGCGCCACAAATTTCTCCCTGCTGAAACAG | |
| | | GCCGGCGACGTGGAAGAGAATCCCGGACCTATGCCCAATCCTA | |
| | | GACCTGGCAAGCCCAGCGCTCCTTCTCTTGCTCTTGGACCTTC | |
| | | TCCTGGTGCAAGTCCATCTTGGCGAGCAGCTCCAAAGGCTAGT | |
| | | GATCTTCTCGGAGCTAGAGGTCCTGGAGGCACATTTGGA | |
| | Full-length | ACTTGCCAGGACTGTTACAATAGCCTCCTCACTAGCCCCACTC | 219 |
| | donor | ACAGCAGCCAGATGAATCTTTTGAGTCCATGCCTAGTCACTGG | |
| | template | GGCAAAATAGGACTCCGAGGAGAAAGTCCGAGACCAGCTCCGG | |
| | | CAAGATGAGCAAACACAGCCTGTGCAGGGTGCAGGGAGGGCTA | |
| | | GAGGCCTGAGGCTTGAAACAGCTCTCAAGTGGAGGGGGAAACA | |
| | | ACCATTGCCCTCATAGAGGACACATCCACACCAGGGCTGTGCT | |
| | | AGCGTGGGCAGGCAAGCCAGGTGCTGGACCTCTGCACGTGGGG | |
| | | CATGTGTGGGTATGTACATGTACCTGTGTTCTTGGTGTGTGTG | |
| | | TGTGTGTGTGTGTGTGTGTGTGTCTAGAGCTGGGGTGCAACTA | |
| | | TGGGGCCCCTCGGGACATGTCCCAGCCAATGCCTGCTTTGACC | |
| | | AGAGGAGTGTCCACGTGGCTCAGGTGGTCGAGTATCTCATACC | |
| | | GCCCTAGCACACGTGTGACTCCTTTCCCCTATTGTCTACGCAG | |
| | | CCTGCCCTTGGACAAGGACCCGATGCCCAACCCCAGGCCTGGC | |
| | | AAGCCCTCGGCCCCTTCCTTGGCCCTTGGCCCATCCCCAGGAG | |
| | | CCTCGCCCAGCTGGAGGGCTGCACCCAAAGCCTCAGACCTGCT | |
| | | GGGGGCCCGGGGCCCAGGGGGAACCTTCCAGTGAACAGAGAAA | |
| | | CAGGAGAATATGGGCCAAACAGGATATCTGTGGTAAGCAGTTC | |
| | | CTGCCCCGGCTCAGGGCCAAGAACAGTTGGAACAGCAGAATAT | |
| | | GGGCCAAACAGGATATCTGTGGTAAGCAGTTCCTGCCCCGGCT | |
| | | CAGGGCCAAGAACAGATGGTCCCCAGATGCGGTCCCGCCCTCA | |
| | | GCAGTTTCTAGAGAACCATCAGATGTTTCCAGGGTGCCCCAAG | |
| | | GACCTGAAATGACCCTGTGCCTTATTTGAACTAACCAATCAGT | |
| | | TCGCTTCTCGCTTCTGTTCGCGCGCTTCTGCTCCCCGAGCTCT | |
| | | ATATAAGCAGAGCTCGTTTAGTGAACCGTCAGATCGCCTGGAG | |
| | | ACGCCATCCACGCTGTTTTGACTTCCATAGAAGGATCTCGCCG | |
| | | CCACCATGCCTCTGGGCCTGCTGTGGCTGGGCCTGGCCCTGCT | |
| | | GGGCGCCCTGCACGCCCAGGCCGAGATGTGGCACGAGGGCCTG | |
| | | GAGGAGGCCAGCAGGCTGTATTTTGGCGAGCGCAACGTGAAGG | |
| | | GCATGTTCGAGGTGCTGGAGCCTCTGCACGCCATGATGGAGAG | |
| | | AGGCCCACAGACCCTGAAGGAGACATCCTTTAACCAGGCCTAT | |
| | | GGACGGGACCTGATGGAGGCACAGGAGTGGTGCAGAAAGTACA | |
| | | TGAAGTCTGGCAATGTGAAGGACCTGACGCAGGCCTGGGATCT | |
| | | GTACTATCACGTGTTTCGGAGAATCTCCAAGGGCAAAGACACG | |
| | | ATTCCGTGGCTTGGGCATCTGCTCGTTGGGCTGAGTGGTGCGT | |
| | | TTGGTTTCATCATCTTGGTCTATCTCTTGATCAATTGCAGAAA | |
| | | TACAGGCCCTTGGCTGAAAAAAGTGCTCAAGTGTAATACCCCC | |
| | | GACCCAAGCAAGTTCTTCTCCCAGCTTTCTTCAGAGCATGGAG | |
| | | GCGATGTGCAGAAATGGCTCTCTTCACCTTTTCCCTCCTCAAG | |
| | | TTTCTCCCCGGGAGGGCTGGCGCCCGAGATTTCACCTCTTGAG | |
| | | GTACTTGAACGAGACAAGGTTACCCAACTTCTCCTTCAACAGG | |
| | | ATAAGGTACCCGAACCTGCGAGCCTTAGCTCCAACCACTCTCT | |
| | | TACGAGCTGCTTCACCAATCAGGGATACTTCTTTTTCCACCTT | |
| | | CCCGATGCGCTGGAAATCGAAGCATGTCAAGTTTACTTTACCT | |
| | | ATGATCCATATAGCGAGGAAGATCCCGACGAAGGAGTCGCCGG | |
| | | TGCGCCCACGGGTTCCTCACCCCAACCTCTCCAGCCTCTCTCA | |
| | | GGAGAAGATGATGCTTATTGCACTTTTCCCAGTAGAGACGATC | |
| | | TCCTCCTCTTTTCTCCATCTCTTTTGGGGGGACCTTCCCCCCC | |
| | | TTCTACGGCACCTGGCGGGTCTGGTGCTGGCGAGGAGCGGATG | |
| | | CCGCCGTCCCTCCAGGAGCGAGTACCACGAGATTGGGATCCCC | |
| | | AGCCACTTGGACCACCAACTCCAGGCGTACCTGACCTTGTCGA | |
| | | TTTTCAACCTCCCCCTGAATTGGTGCTGCGAGAGGCTGGGGAG | |
| | | GAAGTTCCGGACGCTGGGCCGAGGGAGGGCGTGTCCTTTCCAT | |
| | | GGAGTAGGCCTCCAGGTCAAGGCGAGTTTAGGGCTCTCAACGC | |
| | | GCGGCTGCCGTTGAATACAGACGCTTATCTCTCACTGCAGGAA | |
| | | CTGCAAGGTCAGGACCCAACACATCTTGTAGGATCCGGCGCCA | |
| | | CCAACTTTAGTCTGCTCAAGCAAGCCGGGGACGTCGAGGAAAA | |
| | | TCCTGGGCCAGAAATGTGGCACGAAGGACTCGAGGAAGCCAGT | |
| | | CGGCTGTATTTTGGCGAGCGGAATGTGAAAGGGATGTTTGAAG | |
| | | TGCTCGAGCCTCTCCACGCTATGATGGAACGGGGACCCCAGAC | |
| | | TCTCAAAGAAACCAGCTTTAATCAGGCTTACGGACGCGACCTC | |
| | | ATGGAAGCTCAAGAATGGTGTAGAAAGTATATGAAGAGTGGCA | |
| | | ACGTGAAAGATCTGACACAAGCCTGGGATCTCTATTATCACGT | |
| | | GTTCAGACGCATCAGCAAAGGCAGCGGCGCCACAAATTTCTCC | |
| | | CTGCTGAAACAGGCCGGCGACGTGGAAGAGAATCCCGGACCTA | |
| | | TGCCCAATCCTAGACCTGGCAAGCCCAGCGCTCCTTCTCTTGC | |
| | | TCTTGGACCTTCTCCTGGTGCAAGTCCATCTTGGCGAGCAGCT | |
| | | CCAAAGGCTAGTGATCTTCTCGGAGCTAGAGGTCCTGGAGGCA | |
| | | CATTTGGACGAGATCTTCGAGGCGGGGCCCATGCCTCCTCTTC | |
| | | TTCCTTGAACCCCATGCCACCATCGCAGCTGCAGGTGAGGCCC | |
| | | TGGGCCCAGGATGGGGCAGGCAGGGTGGGGTACCTGGACCTAC | |
| | | AGGTGCCGACCTTTACTGTGGCACTGGGCGGGAGGGGGGCTGG | |
| | | CTGGGGCACAGGAAGTGGTTTCTGGGTCCCAGGCAAGTCTGTG | |
| | | ACTTATGCAGATGTTGCAGGGCCAAGAAAATCCCCACCTGCCA | |
| | | GGCCTCAGAGATTGGAGGCTCTCCCCGACCTCCCAATCCCTGT | |
| | | CTCAGGAGAGGAGGAGGCCGTATTGTAGTCCCATGAGCATAGC | |
| | | TATGTGTCCCCATCCCCATGTGACAAGAGAAGAGGACTGGGGC | |
| | | CAAGTAGGTGAGGTGACAGGGCTGAGGCCAGCTCTGCAACTTA | |
| | | TTAGCTGTTTGATCTTTAAAAAGTTACTCGATCTCCATGAGCC | |
| | | TCAGTTTCCATACGTGTAAAAGGGGGATGATCATAGCATCTAC | |
| | | CATGTGGGCTTGCAGTGCAGAGTATTTGAATTAGACACAGAAC | |
| | | AGTGAGGATCAGGATGGCCTCTCACCCACCTGCCTTTCTGCCC | |
| | | AGCTGCCCACACTGCCCCTAGTCATGGTGGCACCCTCCGGGGC | |
| | | ACGGCTGGGCCCCT | |

TABLE 8
Examples of gRNAs for targeted
cleavage in a FOXP3 locus

| | Protospacer | SEQ | Protospacer |
|---|---|---|---|
| gRNA | Sequence | ID | adjacent |
| # | of gRNA | NO: | motif (PAM) |
| 1 | UCCAGCUGGGCGAGGCUCCU | 241 | GGG |
| 2 | UCGAAGAUCUCGGCCCUGGA | 242 | AGG |
| 3 | CGCCUCGAAGAUCUCGGCCC | 243 | TGG |
| 4 | GGGCCGAGAUCUUCGAGGCG | 244 | GGG |

TABLE 9
Examples of other sequences referenced in methods, cells, and systems
described herein

| | | SEQ |
|---|---|---|
| | | ID |
| Sequence Name | Sequence | NO: |
| MND Promoter | GAACAGAGAAACAGGAGAATATGGGCCAAACAGGATATCTGTGGTAAGC | 220 |
| | AGTTCCTGCCCCGGCTCAGGGCCAAGAACAGTTGGAACAGCAGAATATG | |
| | GGCCAAACAGGATATCTGTGGTAAGCAGTTCCTGCCCCGGCTCAGGGCC | |
| | AAGAACAGATGGTCCCCAGATGCGGTCCCGCCCTCAGCAGTTTCTAGAG | |
| | AACCATCAGATGTTTCCAGGGTGCCCCAAGGACCTGAAATGACCCTGTG | |
| | CCTTATTTGAACTAACCAATCAGTTCGCTTCTCGCTTCTGTTCGCGCGC | |
| | TTCTGCTCCCCGAGCTCTATATAAGCAGAGCTCGTTTAGTGAACCGTCA | |
| | GATC | |
| T2A Nucleotide | GAGGGCAGAGGAAGTCTGCTAACATGCGGTGACGTCGAGGAGAATCCTG | 221 |
| Sequence | GACCT | |
| T2A Amino Acid | EGRGSLLTCGDVEENPGP | 222 |
| Sequence | | |
| P2A-1 (TRAC) | GCTACTAACTTCAGCCTGCTGAAGCAGGCTGGAGACGTGGAGGAGAACC | 223 |
| Nucleotide | CTGGACCT | |
| Sequence | | |
| P2A-2 (FOXP3-1) | GCCACCAACTTTAGTCTGCTCAAGCAAGCCGGGGACGTCGAGGAAAATC | 224 |
| Nucleotide | CTGGGCCA | |
| Sequence | | |
| P2A-3 (FOXP3-2) | GCCACAAATTTCTCCCTGCTGAAACAGGCCGGCGACGTGGAAGAGAATC | 225 |
| Nucleotide | CCGGACCT | |
| Sequence | | |
| P2A-1 Amino Acid | ATNFSLLKQAGDVEENPGP | 226 |
| Sequence | | |
| P2A-2 Amino Acid | ATNFSLLKQAGDVEENPGP | 227 |
| Sequence | | |
| P2A-3 Amino Acid | ATNFSLLKQAGDVEENPGP | 228 |
| Sequence | | |
| Linker | GGGS | 229 |
| Linker | GGGSGGG | 230 |
| IGRP 305-324 | QLYHELQIPTHEEHLFYVLS | 231 |
| IGRP 241-260 | KWCANPDWIHIDTTPFAGLV | 232 |
| GAD65 533-572 | KVNFFRMVISNPAATHQDID | 233 |
| GAD65 265-284 | KGMAALPRLIAFTSEHSHES | 234 |
| Preproinsulin | SLQPLALEGSLQKRG | 235 |
| 76-90 | | |
| Wild-type mTOR | MLGTGPAAATTAATTSSNVSVLQQFASGLKSRNEETRAKAAKELQHYVT | 236 |
| | MELREMSQEESTRFYDQLNHHIFELVSSSDANERKGGILAIASLIGVEG | |
| | GNATRIGRFANYLRNLLPSNDPVVMEMASKAIGRLAMAGDTFTAEYVEF | |
| | EVKRALEWLGADRNEGRRHAAVLVLRELAISVPTFFFQQVQPFFDNIFV | |
| | AVWDPKQAIREGAVAALRACLILTTQREPKEMQKPQWYRHTFEEAEKGF | |
| | DETLAKEKGMNRDDRIHGALLILNELVRISSMEGERLREEMEEITQQQL | |
| | VHDKYCKDLMGFGTKPRHITPFTSFQAVQPQQSNALVGLLGYSSHQGLM | |
| | GFGTSPSPAKSTLVESRCCRDLMEEKFDQVCQWVLKCRNSKNSLIQMTI | |
| | LNLLPRLAAFRPSAFTDTQYLQDTMNHVLSCVKKEKERTAAFQALGLLS | |
| | VAVRSEFKVYLPRVLDIIRAALPPKDFAHKRQKAMQVDATVFTCISMLA | |
| | RAMGPGIQQDIKELLEPMLAVGLSPALTAVLYDLSRQIPQLKKDIQDGL | |
| | LKMLSLVLMHKPLRHPGMPKGLAHQLASPGLTTLPEASDVGSITLALRT | |
| | LGSFEFEGHSLTQFVRHCADHELNSEHKEIRMEAARTCSRLLTPSIHLI | |
| | SGHAHVVSQTAVQVVADVLSKLLVVGITDPDPDIRYCVLASLDERFDAH | |
| | LAQAENLQALFVALNDQVFEIRELAICTVGRLSSMNPAFVMPFLRKMLI | |
| | QILTELEHSGIGRIKEQSARMLGHLVSNAPRLIRPYMEPILKALILKLK | |
| | DPDPDPNPGVINNVLATIGELAQVSGLEMRKWVDELFIIIMDMLQDSSL | |
| | LAKRQVALWTLGQLVASTGYVVEPYRKYPTLLEVLLNELKTEQNQGTRR | |
| | EAIRVLGLLGALDPYKHKVNIGMIDQSRDASAVSLSESKSSQDSSDYST | |
| | SEMLVNMGNLPLDEFYPAVSMVALMRIFRDQSLSHHHTMVVQAITFIFK | |
| | SLGLKCVQFLPQVMPTFLNVIRVCDGAIREFLFQQLGMLVSFVKSHIRP | |
| | YMDEIVTLMREFWVMNTSIQSTIILLIEQIVVALGGEFKLYLPQLIPHM | |
| | LRVFMHDNSPGRIVSIKLLAAIQLFGANLDDYLHLLLPPIVKLEDAPEA | |
| | PLPSRKAALETVDRLTESLDETDYASRIIHPIVRTLDQSPELRSTAMDT | |
| | LSSLVFQLGKKYQIFIPMVNKVLVRHRINHQRYDVLICRIVKGYTLADE | |
| | EEDPLIYQHRMLRSGQGDALASGPVETGPMKKLHVSTINLQKAWGAARR | |
| | VSKDDWLEWLRRLSLELLKDSSSPSLRSCWALAQAYNPMARDLENAAFV | |
| | SCWSELNEDQQDELIRSIELALTSQDIAEVTQTLLNLAEFMEHSDKGPL | |
| | PLRDDNGIVLLGERAAKCRAYAKALHYKELEFQKGPTPAILESLISINN | |
| | KLQQPEAAAGVLEYAMKHFGELEIQATWYEKLHEWEDALVAYDKKMDTN | |
| | KDDPELMLGRMRCLEALGEWGQLHQQCCEKWTLVNDETQAKMARMAAAA | |
| | AWGLGQWDSMEEYTCMIPRDTHDGAFYRAVLALHQDLFSLAQQCIDKAR | |
| | DLLDAELTAMAGESYSRAYGAMVSCHMLSELEEVIQYKLVPERREIIRQ | |
| | IWWERLQGCQRIVEDWQKILMVRSLVVSPHEDMRTWLKYASLCGKSGRL | |
| | ALAHKTLVLLLGVDPSRQLDHPLPTVHPQVTYAYMKNMWKSARKIDAFQ | |
| | HMQHFVQTMQQQAQHAIATEDQQHKQELHKLMARCELKLGEWQLNLQGI | |
| | NESTIPKVLQYYSAATEHDRSWYKAWHAWAVMNFEAVLHYKHQNQARDE | |
| | KKKLRHASGANITNATTAATTAATATTTASTEGSNSESEAESTENSPTP | |
| | SPLQKKVTEDLSKTLLMYTVPAVQGEFRSISLSRGNNLQDTLRVLTLWE | |
| | DYGHWPDVNEALVEGVKAIQIDTWLQVIPQLIARIDTPRPLVGRLIHQL | |
| | LTDIGRYHPQALIYPLTVASKSTTTARHNAANKILKNMCEHSNTLVQQA | |
| | MMVSEELIRVAILWHEMWHEGLEEASRLYFGERNVKGMFEVLEPLHAMM | |
| | ERGPQTLKETSFNQAYGRDLMEAQEWCRKYMKSGNVKDLTQAWDLYYHV | |
| | FRRISKQLPQLTSLELQYVSPKLLMCRDLELAVPGTYDPNQPIIRIQSI | |
| | APSLQVITSKQRPRKLTLMGSNGHEFVFLLKGHEDLRQDERVMQLFGLV | |
| | NTLLANDPTSLRKNLSIQRYAVIPLSTNSGLIGWVPHCDTLHALIRDYR | |
| | EKKKILLNIEHRIMLRMAPDYDHLTLMQKVEVFEHAVNNTAGDDLAKLL | |
| | WLKSPSSEVWEDRRTNYTRSLAVMSMVGYILGLGDRHPSNLMLDRLSGK | |
| | ILHIDEGDCFEVAMTREKFPEKIPFRLTRMLTNAMEVTGLDGNYRITCH | |
| | TVMEVLREHKDSVMAVLEAFVYDPLLNWRLMDTNTKGNKRSRTRTDSYS | |
| | AGQSVEILDGVELGEPAHKKTGTTVPESIHSFIGDGLVKPEALNKKAIQ | |
| | IINRVRDKLTGRDESHDDTLDVPTQVELLIKQATSHENLCQCYIGWCPF | |
| | W | |
| gRNA 1 | NNNNNNNNNNNNNNNNNNNNGUUUUAGAGCUAGAAAUAGCAAGUUAAAA | 237 |
| | UAAGGCUAGUCCGUUAUCAACUUGAAAAAGUGGCACCGAGUCGGUGCUU | |
| | UU | |
| gRNA 2 | NNNNNNNNNNNNNNNNNNNNNNNNNGUCAUAGUUCCAUUAAAGCCAAAA | 238 |
| | GUGGCUUUGAUGUUUCUAUGAUAAGGGUUUCGACCCGUGGCGUCGGGGA | |
| | UCGCCUGCCCAUUGAAAUGGGCUUCUCCCCAUUUAUU | |
| gRNA 3 | NNNNNNNNNNNNNNNNNNNNNNNNNGUCAUAGUUCCAUGAAAGCCAAAA | 239 |
| | GUGGCUUUGAUGUUUCUAUGAUAAGGGUUUCGGCCCGUGGCGUCGGGGA | |
| | UCGCCUGCCCAUUCCGAUGGGCUUCUCCCCAUUUAUU | |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### Additional Embodiments

1. A gene editing chemical-inducible signaling complex (CISC) system comprising: a first polynucleotide: a first promoter, wherein the first promoter is MND, a first nucleic acid encoding a first CISC component comprising rapamycin binding domain of FK-binding protein 12 (FKBP) or a functional fragment thereof, and an intracellular signaling domain or functional fragment of IL2Rγ, a nucleic acid encoding TCRβ or a functional fragment thereof, and a nucleic acid encoding TRAV and/or TRAJ or functional fragment thereof, wherein the TCRβ and TRAV and/or TRAJ form parts of a TCR specific to a type 1 diabetes (T1D) antigen; and a second polynucleotide comprising: a second promoter, wherein the second promoter is MND, a second nucleic acid encoding a second CISC component comprising a second extracellular binding domain that comprises rapamycin binding domain of FKBP12-Rapamycin Binding domain of mTOR (FRB) or functional fragment thereof, and a second intracellular signaling domain comprising an intracellular signaling domain or functional fragment of IL2Rβ, and a third nucleic acid encoding a cytosolic FRB or function fragment thereof.
2. The CISC system of Additional Embodiment 1, wherein the first polynucleotide further comprises a first 5′ homology arm and a first 3′ homology arm that each share more than 80% sequence identity to genomic sequence in a first locus, wherein the first locus is a TRAC locus, and the second polynucleotide further comprises a second 5′ homology arm and a 3′ homology arm that each share more than 80% sequence identity to genomic sequence in a second locus, wherein the second locus is a Foxp3 locus.
3. The CISC system of Additional Embodiment 1 or Additional Embodiment 2, wherein the first polynucleotide further comprises a nucleic acid encoding a 2A self-cleaving peptide between the first nucleic acid encoding a first CISC component and the nucleic acid encoding TCRβ or a functional fragment thereof, a nucleic acid encoding a 2A self-cleaving peptide between the nucleic acid encoding TCRβ or a functional fragment thereof and the nucleic acid encoding TRAV and/or TRAJ or functional fragment thereof.
4. The CISC system of any one of the preceding Additional Embodiments, wherein the MND comprises the sequence of SEQ ID NO: 1.
5. The CISC system of any one of the preceding Additional Embodiments, wherein the first nucleic acid encoding a first CISC component comprises the sequence of SEQ ID NO: 2.
6. The CISC system of any one of the preceding Additional Embodiments, wherein the nucleic acid encoding TCRβ or a functional fragment thereof comprises the sequence of SEQ ID NO: 3 or the sequence of SEQ ID NO: 5.
7. The CISC system of any one of the preceding Additional Embodiments, wherein the nucleic acid encoding TRAV and/or TRAJ or functional fragment thereof comprises the sequence of SEQ ID NO: 4 or SEQ ID NO: 6.
8. The CISC system of any one of the preceding Additional Embodiments, wherein the second nucleic acid encoding a second CISC component comprises the sequence of SEQ ID NO: 11.
9. The CISC system of any one of the preceding Additional Embodiments, wherein the third nucleic acid encoding a cytosolic FRB or function fragment thereof comprises the sequence of SEQ ID NO: 12.
10. The CISC system of any one of the preceding Additional Embodiments, wherein the first polynucleotide comprises the sequence of SEQ ID NO: 15 or the sequence of SEQ ID NO: 16.
11. The CISC system of any one of the preceding Additional Embodiments, wherein the second polynucleotide comprises the sequence of SEQ ID NO: 17.
12. The CISC system of any one of the preceding Additional Embodiments, wherein the first polynucleotide comprises the sequence of SEQ ID NO: 15 or the sequence of SEQ ID NO: 16, and the second polynucleotide comprises the sequence of SEQ ID NO: 17.
13. A method of engineering a population of Treg cells, the method comprising: (a) dual-editing a population of T cells isolated from a first subject by contacting the population of T cells with (i) a first polynucleotide comprising: a first promoter, wherein the first promoter is MND, a first nucleic acid encoding a first CISC component comprising rapamycin binding domain of FK-binding protein 12 (FKBP) or a functional fragment thereof, and an intracellular signaling domain or functional fragment of IL2Rγ, a nucleic acid encoding TCRβ or a functional fragment thereof, and a nucleic acid encoding TRAV and/or TRAJ or functional fragment thereof, wherein the TCRβ and TRAV and/or TRAJ form parts of a TCR specific to a type 1 diabetes (T1D) antigen; (ii) a second polynucleotide comprising: a second promoter, wherein the second promoter is MND, a second nucleic acid encoding a second CISC component comprising a second extracellular binding domain that comprises rapamycin binding domain of FKBP12-Rapamycin Binding domain of mTOR (FRB) or functional fragment thereof, and a first intracellular signaling domain comprising an intracellular signaling domain or functional fragment of IL2Rβ, and a third nucleic acid encoding a cytosolic FRB or function fragment thereof; (iii) a first endonuclease, or nucleic acid encoding the first endonuclease, that can cleave within a first locus, wherein the first locus is a TRAC locus, (iv) a second endonuclease, or nucleic acid encoding the second endonuclease, that can cleave a within a second locus, wherein the second locus is a Foxp3 locus, such that the first polynucleotide or fragment thereof is incorporated into the first locus, and the second polynucleotide or fragment thereof is inserted in the second locus; and (b) selectively expanding a subpopulation of dual-edited cells in the population of T cells by contacting the population of T cells with rapamycin, wherein the subpopulation of dual-edited cells have a suppressive phenotype.
14. The method of Additional Embodiment 13, wherein the population of T cells is contacted with 0.01-100 nM rapamycin.
15. The method of Additional Embodiment 14, wherein the population of T cells is contacted with 10 nM.
16. The method of any one of Additional Embodiments 13-15, further comprising isolating from a first subject a population of T cells, wherein the population of T cells are isolated from the subject's blood.
17. The method of any one of Additional Embodiments 13-16, wherein the first subject is human.
18. The method of any one of Additional Embodiments 13-17, further comprising administering to a second subject an aliquot of the population of T cells comprising the selectively expanded subpopulation of dual-edited cells having a suppressive phenotype, wherein the second subject suffers from or is at risk of suffering from diabetes.
19. A method for treating, inhibiting, or ameliorating T1D, the method comprising administering to a subject a composition comprising dual-edited cells having a suppressive phenotype, wherein the dual-edited cells comprise: (i) a first polynucleotide inserted within the TRAC locus, the first polynucleotide comprising: a first promoter, wherein the first promoter is MND, a first nucleic acid encoding a first CISC component comprising rapamycin binding domain of FK-binding protein 12 (FKBP) or a functional fragment thereof, and an intracellular signaling domain or functional fragment of IL2Rγ, a nucleic acid encoding TCRβ or a functional fragment thereof, and a nucleic acid encoding TRAV and/or TRAJ or functional fragment thereof, wherein the TCRβ and TRAV and/or TRAJ form parts of a TCR specific to a type 1 diabetes (T1D) antigen; (ii) a second polynucleotide inserted within the Foxp3 locus, the second polynucleotide comprising: a second promoter, wherein the second promoter is MND, a second nucleic acid encoding a second CISC component comprising a second extracellular binding domain that comprises rapamycin binding domain of FKBP12-Rapamycin Binding domain of mTOR (FRB) or functional fragment thereof, and a first intracellular signaling domain comprising an intracellular signaling domain or functional fragment of IL2Rβ, and a third nucleic acid encoding a cytosolic FRB or function fragment thereof.
20. The method of any one of the Additional Embodiments 18 or 19, further comprising administering to the second subject rapamycin.
21. The method of any one of Additional Embodiments 18-20, wherein the first subject and the second subject are the same.
22. The method of any one of Additional Embodiments 18-21, wherein the second subject is human.
23. A population of cells comprising a subpopulation of dual-edited cells having a suppressive phenotype, wherein the population of cells is made by the method of any one of Additional Embodiments 13-21.
24. A population of cells comprising a subpopulation of dual-edited cells having a suppressive phenotype, wherein the subpopulation comprises at least 10% of the population of T cells within 2 days of being dual-edited, and wherein the subpopulation of cells comprises cells comprising: (i) a first polynucleotide inserted within the TRAC locus, the first polynucleotide comprising: a first promoter, wherein the first promoter is MND, a first nucleic acid encoding a first CISC component comprising rapamycin binding domain of FK-binding protein 12 (FKBP) or a functional fragment thereof, and an intracellular signaling domain or functional fragment of IL2Rγ, a nucleic acid encoding TCRβ or a functional fragment thereof, and a nucleic acid encoding TRAV and/or TRAJ or functional fragment thereof, wherein the TCRβ and TRAV and/or TRAJ form parts of a TCR specific to a type 1 diabetes (T1D) antigen; (ii) a second polynucleotide inserted within the Foxp3 locus, the second polynucleotide comprising: a second promoter, wherein the second promoter is MND, a second nucleic acid encoding a second CISC component comprising a second extracellular binding domain that comprises rapamycin binding domain of FKBP12-Rapamycin Binding domain of mTOR (FRB) or functional fragment thereof, and a first intracellular signaling domain comprising an intracellular signaling domain or functional fragment of IL2Rβ, and a third nucleic acid encoding a cytosolic FRB or function fragment thereof.
25. The population of cells of Additional Embodiment 24, wherein the subpopulation comprises at least 60% of the population of T cells within 18 days of being dual-edited.
26. A nucleic acid encoding a TCR, wherein the nucleic acid comprises the sequence of SEQ ID NO: 3 and the sequence of SEQ ID NO: 4, or the sequence of SEQ ID NO: 5 and the sequence of SEQ ID NO: 6.
27. The nucleic acid of Additional Embodiment 24, wherein the nucleic acid comprises the sequence of SEQ ID NO: 19, or the sequence of SEQ ID NO: 20.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1.-174. (canceled)

175. A genetically modified regulatory T (Treg) cell comprising:
(i) a first inserted nucleic acid in a TRAC locus of the cell genome, wherein the TRAC locus comprises:
(a) a first MND promoter;
(b) an exogenous nucleotide sequence encoding a first chemically induced signaling complex (CISC) component comprising:
(1) an extracellular binding domain comprising a rapamycin-binding domain of FK506-binding protein 12 (FKBP),
(2) an IL-2Rγ transmembrane domain, and
(3) an intracellular domain comprising an IL-2Rγ cytoplasmic domain a functional fragment thereof;
(c) an exogenous nucleotide sequence encoding an exogenous TCRβ polypeptide or a functional fragment thereof, and
(d) an exogenous nucleotide sequence encoding at least a portion of a TCRα polypeptide,
wherein the portion of the TCRα polypeptide comprises a TCRα variable (TRAV) region and TCRα joining (TRAJ) region,
wherein the nucleotide sequence encoding the TCRα variable and joining regions is inserted in-frame with an endogenous nucleotide sequence encoding a portion of a TCRα constant domain, such that the first MND promoter initiates transcription of a nucleotide sequence encoding the exogenous TCRβ polypeptide and a nucleotide sequence encoding a TCRα polypeptide comprising heterologous TRAV/TRAJ amino acid sequences and an endogenous TCRα constant domain,
wherein a T cell receptor (TCR) comprising the TCRα and TCRβ polypeptides binds to a type 1 diabetes (T1D)-associated antigen, and
wherein the TCRα polypeptide comprises an αCDR1 having the amino acid sequence of SEQ ID NO: 1, an αCDR2 having the amino acid sequence of SEQ ID NO: 2, and an αCDR3 having the amino acid sequence of SEQ ID NO: 3, and the TCRβ polypeptide comprises a βCDR1 having the amino acid sequence of SEQ ID NO: 4, a βCDR2 having the amino acid sequence of SEQ ID NO: 5, and a βCDR3 having the amino acid sequence of SEQ ID NO: 6; and
(ii) a second inserted nucleic acid in a FOXP3 locus of the cell genome, wherein the FOXP3 locus comprises:
(a) a second MND promoter;
(b) a nucleotide sequence encoding a second CISC component comprising:
(1) an extracellular binding domain comprising an FKBP-rapamycin-binding (FRB) domain of mTOR;
(2) an IL-2Rβ transmembrane domain, and
(3) an intracellular domain comprising an IL-2Rβ cytoplasmic domain or a functional fragment thereof; and
(c) a nucleotide sequence encoding a cytosolic FRB domain that binds rapamycin and does not comprise a transmembrane domain,
wherein the second MND promoter is inserted downstream from a Treg-specific demethylated region of the FOXP3 locus, and initiates transcription of an endogenous nucleotide sequence encoding FOXP3 or a portion thereof, and
wherein the genetically modified Treg cell is a CD4+ cell.

176. The genetically modified Treg cell of claim 175, wherein the TCRα polypeptide comprises the amino acid sequence of SEQ ID NO: 9, and the TCRβ polypeptide comprises the amino acid sequence of SEQ ID NO: 10.

177. The genetically modified Treg cell of claim 175, wherein the first and second CISC components form a heterodimer in the presence of rapamycin or a rapalog.

178. The genetically modified Treg cell of claim 177, wherein the rapalog is everolimus, CCI-779, C20-methallylrapamycin, C16-(S)-3-methylindolerapamycin, C16-iRap, C16-(S)-7-methylindolerapamycin, AP21967, C16-(S)Butylsulfonamidorapamycin, AP23050, sodium mycophenolic acid, benidipine hydrochloride, AP1903, AP23573, a metabolite of any one of the foregoing rapalogs, or a derivative of any one of the foregoing rapalogs.

179. A pharmaceutical composition comprising the genetically modified Treg cell of claim 175 and a pharmaceutically acceptable excipient.

180. A method of treating, ameliorating, or inhibiting type 1 diabetes in a subject in need thereof, the method comprising administering a therapeutically effective amount of the genetically modified Treg cell of claim 175 to the subject.

181. The method of claim 180, wherein the genetically modified Treg cell is autologous to the subject.

182. The method of claim 180, wherein the method further comprises administering rapamycin to the subject before administration of the genetically modified cell, in conjunction with administration of the genetically modified cell, and/or following the administration of the genetically modified cell.

183. The method of claim 182, wherein the rapamycin is administered at a dose of 0.01 mg/kg to 0.1 mg/kg.

184. A method of producing a genetically modified cell, the method comprising contacting a CD4+ cell with:
(i) a first nucleic acid comprising:
(a) a first 5′ homology arm having homology to a first nucleic acid sequence in a TRAC locus in the cell genome;
(b) a first MND promoter;
(c) a nucleotide sequence encoding a first chemically induced signaling complex (CISC) component comprising:
(1) an extracellular binding domain comprising a rapamycin-binding domain of FK506-binding protein 12 (FKBP),
(2) an IL-2Rγ transmembrane domain, and
(3) an intracellular domain comprising an IL-2Rγ cytoplasmic domain or a functional fragment thereof;
(d) a nucleotide sequence encoding a TCRβ polypeptide or a functional fragment thereof;
(e) a nucleotide sequence encoding at least a portion of a TCRα polypeptide,
wherein the portion of the TCRα polypeptide comprises a TCRα variable (TRAV) region and TCRα joining (TRAJ) region,
wherein the nucleotide sequence encoding the TCRα variable and joining regions is inserted in-frame with an endogenous nucleotide sequence encoding a portion of a TCRα constant domain, such that the first MND promoter initiates transcription of a nucleotide sequence encoding the TCRβ polypeptide and a nucleotide sequence encoding a TCRα polypeptide comprising heterologous TRAV/TRAJ amino acid sequences and an endogenous TCRα constant domain,
wherein a T cell receptor (TCR) comprising the TCRα and TCRβ polypeptides binds to a type 1 diabetes (T1D)-associated antigen, and
wherein the TCRα polypeptide comprises an αCDR1 having the amino acid sequence of SEQ ID NO: 1, an αCDR2 having the amino acid sequence of SEQ ID NO: 2, and an αCDR3 having the amino acid sequence of SEQ ID NO: 3, and the TCRβ polypeptide comprises a βCDR1 having the amino acid sequence of SEQ ID NO: 4, a βCDR2 having the amino acid sequence of SEQ ID NO: 5, and a βCDR3 having the amino acid sequence of SEQ ID NO: 6; and
(f) a first 3′ homology arm having homology to a second nucleic acid sequence in the TRAC locus that is downstream from the first nucleic acid sequence in the TRAC locus;
(ii) a second nucleic acid comprising:
(a) a second 5′ homology arm having homology to a first nucleic acid sequence in a FOXP3 locus in the cell genome;
(b) a second MND promoter;
(c) a nucleotide sequence encoding a second CISC component comprising:
(1) an extracellular binding domain comprising an FKBP-rapamycin-binding (FRB) domain of mTOR;
(2) an IL-2Rβ transmembrane domain, and
(3) an intracellular domain comprising an IL-2Rβ cytoplasmic domain or a functional fragment thereof;
(d) a nucleotide sequence encoding a cytosolic FRB domain that binds rapamycin and does not comprise a transmembrane domain; and
(e) a second 3′ homology arm having homology to a second nucleic acid sequence in the FOXP3 locus that is downstream from the first nucleic acid sequence in the FOXP3 locus, and downstream from a Treg-specific demethylated region (TSDR) in the FOXP3 locus;
(iii) an RNA-guided DNA endonuclease or nucleic acid encoding the RNA-guided DNA endonuclease;
(iv) a TRAC locus-targeting guide RNA (gRNA) comprising a spacer sequence that is complementary to a sequence within the TRAC locus or nucleic acid encoding the TRAC locus-targeting gRNA; and
(v) a FOXP3 locus-targeting guide RNA (gRNA) comprising a spacer sequence that is complementary to a sequence within the FOXP3 locus or nucleic acid encoding the FOXP3 locus-targeting gRNA.

185. The method of claim 184, wherein the TCRα polypeptide comprises the amino acid sequence of SEQ ID NO: 9, and the TCRβ polypeptide comprises the amino acid sequence of SEQ ID NO: 10.

186. The method of claim 184, wherein the RNA-guided DNA endonuclease is Cas9.

187. The method of claim 184, wherein the genetically modified cell is a regulatory T (Treg) cell.

188. A genetically modified Treg made by the method of claim 187.

189. The method of claim 184, wherein the first and second CISC components form a heterodimer in the presence of rapamycin or a rapalog.

190. The method of claim 189, wherein the rapalog is everolimus, CCI-779, C20-methallylrapamycin, C16-(S)-3-methylindolerapamycin, C16-iRap, C16-(S)-7-methylindolerapamycin, AP21967, C16-(S)Butylsulfonamidorapamycin, AP23050, sodium mycophenolic acid, benidipine hydrochloride, AP1903, or AP23573, a metabolite of any one of the foregoing rapalogs, or a derivative of any one of the foregoing rapalogs.

191. The method of claim 189, wherein the genetically modified cell is a regulatory T (Treg) cell.

192. The method of claim 191, wherein the method further comprises contacting the Treg cell with rapamycin.

193. A population of genetically modified regulatory T (Treg) cells made by the method of claim 192.
